(19) United States
(12) Patent Application Publication
(10) Pub. No.: US 2025/0376736 A1
(43) Pub. Date: Dec. 11, 2025

(54) TRANSGENIC COTTON EVENT GH_CSM63718 AND COMPOSITIONS AND METHODS FOR DETECTION AND USES THEREOF

(71) Applicant: Monsanto Technology LLC, St. Louis, MO (US)

(72) Inventors: Sarah Brown, Weldon Spring, MO (US); Zhihuan Gao, O'Fallon, MO (US); Jessica Koczan, Olivette, MO (US); Brian Krebel, St. Louis, MO (US); Aihong Pan, St. Louis, MO (US); Qungang Qi, St. Charles, MO (US); Linda Rymarquis, High Ridge, MO (US); Xiaoping Wei, Apex, NC (US)

(21) Appl. No.: 19/211,863

(22) Filed: May 19, 2025

Related U.S. Application Data
(60) Provisional application No. 63/656,474, filed on Jun. 5, 2024.

(51) Int. Cl. C12Q 1/6895; A01H 1/04; C12N 15/82; G01N 33/569; G01N 33/68

(52) U.S. Cl. CPC C12Q 1/6895; A01H 1/045; C12N 15/8201; C12N 15/8275; G01N 33/56961; G01N 33/6857; C12Q 2600/13; G01N 2333/415

(57) ABSTRACT

A transgenic cotton event, Gh_CSM63718, is provided. Transgenic plant cells, plant parts, plants, seeds, progeny plants, and agricultural and commodity products containing event Gh_CSM63718 are also provided. Recombinant DNA molecules unique to the event Gh_CSM63718, and methods of using and detecting Gh_CSM63718 are also provided. Cotton plants containing the event Gh_CSM63718 exhibit tolerance to glufosinate, ß-triketone HPPD inhibitors, dicamba, glyphosate, PPO inhibitors, and combinations of any thereof.

### CROSS-REFERENCE TO RELATED APPLICATIONS

This application claims priority to U.S. Provisional Application Ser. No. 63/656,474, filed Jun. 5, 2024, the entire disclosure of which is incorporated herein by reference.

### INCORPORATION OF SEQUENCE LISTING

The sequence listing contained in the file named “MONS585US_ST26.xml”, which is 349,651 bytes (measured in MS-Windows) and was created on May 9, 2025, is filed herewith by electronic submission, and is incorporated herein by reference in its entirety.

### FIELD

The present disclosure relates to compositions and methods for providing herbicide tolerance in transgenic cotton plants. Recombinant DNA molecules present in and/or isolated from cotton event Gh_CSM63718 are provided. Also provided are transgenic cotton plants, plant parts, seeds, cells, and agricultural products comprising the cotton event Gh_CSM63718, as well as methods of producing and using transgenic cotton plants, plant parts, seeds, cells, and agricultural products comprising cotton event Gh_CSM63718, methods of detecting cotton event Gh_CSM63718, and methods of controlling weeds. Transgenic cotton plants, plant parts, seeds and cells comprising cotton event Gh_CSM63718 exhibit tolerance to benzoic acid auxins such as dicamba; inhibitors of glutamine synthetase such as glufosinate; ß-triketone herbicides (inhibitors of 4-hydroxyphenylpyruvate dioxygenase, or HPPD) such as mesotrione, inhibitors of 5-enolpyruvylshikimate-3-phosphate synthase (EPSPS) such as glyphosate, and a variety of PPO herbicides.

### BACKGROUND

Increasing sustainable crop production is crucial to meet the need for food for the growing global population, feed for increased demand on animal-based diets in developing nations, and expanded use of crop products to produce biofuel, fiber, and other agricultural product-based commodities, while using limited natural resources. In agricultural systems, the effective management of weedy species in agricultural fields is essential for maintaining favorable crop growing conditions and yield. Weeds compete with crops for space, nutrients, water, and sunlight and can lead to 25-80% yield losses. Selective herbicides had significantly contributed to weed management before the deployment of herbicide tolerant crops. Application of herbicides provides an important tool to reduce weed pressure, improve productivity and increase security for global crop production.

Cotton is a versatile commodity used in many products, particularly clothing. Each year about 27 million tons of cotton is produced globally. The introduction of genetically modified crops containing herbicide tolerance traits has successfully provided additional tools available to farmers to better control weeds. Transgenic herbicide tolerance enables the use of an herbicide in a crop growing environment without crop injury or with minimal crop injury (e.g., less than about 10% injury). Transgenic cotton traits have been used to impart tolerance to glyphosate and glufosinate and are used broadly in commercial cotton production for weed management. However, weeds have evolved resistance to herbicides and weed resistance continues to present a challenge in cotton production today. Therefore, there is a need for additional herbicide tolerance trait options to manage weeds effectively and to sustain crop productivity. One of the solutions is to employ herbicide(s) with new or different mode(s) of action, and/or employ multiple herbicides with different modes of action.

Combinations of herbicide tolerance traits are desirable to provide weed control options that increase grower flexibility and enable the use of multiple herbicide modes of action for controlling challenging weeds. Combining multiple desired traits in the genome can be achieved by several approaches: 1) by making crosses between two parents each having a desired trait at a randomly inserted site, and identifying progeny plants that have combination of the desired traits; 2) by retransforming a transgenic plant comprising one or more desired trait(s) with one or more genes for additional desired traits, either through random integration or through targeted integration of the one or more genes for additional desired traits; 3) by inserting multiple genes as a single DNA molecule into one location, or locus, in the genome, which provides a useful tool in weed control that is much simpler and less expensive to maintain during subsequent breeding into a diverse pool of elite germplasms; and 4) by targeting one or more desired traits to a specific genomic location (site directed integration) carrying one or more desired traits, in a new transformation event, followed by crosses between the new event and another event carrying the one or more desired event at the specific genomic location, resulting in progeny plants that have combination of the desired traits at one location and segregate together.

The expression of transgenes in a transgenic plant, plant part, seed, cell or progeny, and thus their effectiveness, may be influenced by many factors, such as the regulatory elements used in the transgenes' expression cassettes, the combination and/or interaction of these regulatory elements, the chromosomal location of the transgene insertion site, the chromatin structure of the genome at or near the transgene insertion site, and the presence or proximity of any endogenous cis and/or trans regulatory elements or genes close to the transgene insertion site. In addition, the performance of the traits in the transgenic plant is further complicated when the transgenic insert comprises multiple expression cassettes such as five independent expression cassettes in the present disclosure, each having a different transgene conferring a distinct trait. These differences or factors may result in variation in the level of transgene expression or in the spatial or temporal pattern of transgene expression among different transgenic insertion events of the same expression cassettes. Furthermore, different transgenic events can also vary in terms of the molecular quality of the events. For example, a transgenic event may contain two or more copies of the transgene insertion at one or more chromosomal locations, or a transgenic insertion may be truncated relative to the intended insertion or contain vector backbone sequences, or a transgene may be inserted into an endogenous gene or in a repeated region. In the case of site directed integration of desired traits, the machinery for site directed integration, such as gRNA or nuclease, which has to be excised from the commercial events, may not be completely removed. Such characteristics may result in undesirable outcomes, such as gene silencing, altered pattern and/or expression of the transgene, and/or altered pattern and/or expression of endogenous genes. There may also be undesirable phenotypic or agronomic differences among different events.

Even in the case of targeted sequence insertion, variability in the level of transgene expression between independent but genetically identical targeted sequence insertion (TSI) events was observed in a subset of transgenic events (Verkest et al., 2019). This expression variability and silencing occurred independently of the transgene sequence and could be attributed to DNA methylation that was further linked to different DNA methylation mechanisms. Transgene integration into targeted loci through Cre-lox mediated recombination has also been reported to produce a large percentage of targeted integration events that showed a partial spatial pattern of transgene expression due to differential silencing (Day et al., 2000). The fact that a considerable variation in transgene expression was observed shows that even when integration events are targeted, selection remains necessary similarly to the practice for random integration events in order to identify targeted insertion events with stable and desirable gene of interest expression over generations.

A commercially useful transgenic event requires that the transgene(s) in the transgenic insert express in the manner necessary for that trait(s) to be successful, and involves rigorous testing, evaluation, and selection. Such tests include in vitro and/or in planta testing different regulatory elements (e.g., promoters, introns, leaders, and 3′ UTRs) and combinations of different regulatory elements for desirable spatial and temporal expression of the transgene(s), as well as examining whether to target the product of the transgene(s) (protein(s)) to subcellular compartments such as chloroplasts to select for the best expression cassette(s). For site directed integration of a transgene, once a targeted insertion strategy/method is chosen, the target sites are identified, screened and selected. The selected combinations of expression cassette(s), targeting sites and gRNA are then used for transformation to produce transgenic plants. It is also important to remove the selection marker gene efficiently from the events post-transformation to avoid regulatory concerns and to maintain sustainability.

For these reasons, the performance of different transformation events from the same transformation construct can vary widely, and the identification of transformation events conferring the most beneficial traits or characteristics without other potential off-types, concerns or marker gene is needed to select a superior event for commercial use. Therefore, a large number of individual transgenic events must be produced and analyzed to select an event having superior commercial properties, which can be a significant undertaking that involves analysis and selection among many different transformation events.

To establish a transgenic event for commercial use requires rigorous molecular characterization, greenhouse testing, and field trials over multiple years, in multiple locations and under a variety of conditions, allowing extensive agronomic, phenotypic, and molecular data to be obtained. The resulting data are then analyzed to select an event that is suitable for commercial purposes. The commercial event, once identified as having the desired transgene expression, molecular characteristics, efficacy and field performance, can then be introgressed into other cotton genetic backgrounds using plant breeding methods. The resulting cotton varieties contain the new traits combined with other desirable qualities such as native traits, disease tolerance traits, insect control traits, high-yielding germplasms or traits, and/or one or more other transgenic herbicide tolerance traits.

### SUMMARY

A recombinant DNA molecule is provided. The recombinant DNA molecule comprises a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. In some embodiments, the recombinant DNA molecule is derived from a cotton plant, seed, plant part, plant cell, progeny plant, or commodity product comprising cotton event Gh_CSM63718, a representative sample of seed comprising the event having been deposited as ATCC Accession No. PTA-127638. In some embodiments, the recombinant DNA molecule is comprised in a cotton plant, seed, plant part, plant cell, or progeny plant comprising cotton event Gh_CSM63718, or a commodity product produced therefrom, a representative sample of seed comprising the event having been deposited as ATCC Accession No. PTA-127638. The recombinant DNA molecule can be formed by the insertion of a heterologous nucleic acid molecule into the genomic DNA of a cotton plant or cotton cell. The recombinant DNA molecule can comprise an amplicon diagnostic for the presence of cotton event Gh_CSM63718.

DNA molecules that function as DNA probes are provided. An example of such a DNA molecule is a DNA molecule comprising a polynucleotide segment of sufficient length to function as a DNA probe that hybridizes specifically under stringent hybridization conditions with cotton event Gh_CSM63718 DNA in a sample. Detecting hybridization of the DNA molecule under the stringent hybridization conditions is diagnostic for the presence of cotton event Gh_CSM63718 in the sample.

Also provided is a DNA molecule comprising a polynucleotide segment of sufficient length to function as a DNA probe specific for detecting in a sample at least one of: a 5′ junction sequence between flanking cotton genomic DNA and the transgenic insert of cotton event Gh_CSM63718; a 3′ junction sequence between the transgenic insert of cotton event Gh_CSM63718 and flanking cotton genomic DNA; SEQ ID NO:9; and a fragment of SEQ ID NO:9 comprising a sufficient length of contiguous nucleotides of SEQ ID NO:9 to identify the sequence as a fragment of the transgenic insert of Gh_CSM63718.

The DNA probe can comprise SEQ ID NO:21. Alternatively or in addition, the DNA probe can comprise a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing. The sample can be derived from a cotton plant, seed, plant part, plant cell, progeny plant, or commodity product.

A pair of DNA molecules is provided. The pair of DNA molecules comprises a first DNA molecule and a second DNA molecule. The first and the second DNA molecules are different from one another, and each comprise a fragment of SEQ ID NO:10 or a complement thereof and function as DNA primers when used together in an amplification reaction with DNA comprising cotton event Gh_CSM63718 to produce an amplicon diagnostic for cotton event Gh_CSM63718 in a sample. For example, the first and the second DNA molecules can comprise SEQ ID NO:19 and SEQ ID NO:20. The amplicon can comprise a nucleotide sequence selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 17,736-17,737 of SEQ ID NO:10. The sample can be derived from a cotton plant, seed, plant part, plant cell, progeny plant, or commodity product.

Methods for detecting the presence of cotton event Gh_CSM63718 in a sample derived from a cotton seed, plant, plant part, plant cell, progeny plant, or commodity product are provided. In a first example of such a method, the method comprises: (a) contacting the sample with any of the DNA molecules that function as a DNA probes specific for cotton event Gh_CSM63718 described herein; (b) subjecting the sample and the DNA molecule that functions as a probe to stringent hybridization conditions; and (c) detecting the hybridization of the DNA molecule that functions as a probe to a DNA molecule in the sample. The hybridization of the DNA molecule that functions as a probe to the DNA molecule in the sample is diagnostic for the presence of cotton event Gh_CSM63718 in the sample.

Another method of detecting the presence of cotton event Gh_CSM63718 in a sample derived from a cotton seed, plant, plant part or plant cell, progeny plant or commodity product is provided. The method comprises: (a) contacting the sample with any of the pairs of DNA molecules that can be used to produce an amplicon diagnostic for cotton event Gh_CSM63718 described herein; (b) performing an amplification reaction sufficient to produce a DNA amplicon; and (c) detecting the presence of the DNA amplicon. The DNA amplicon comprises at least one of: a 5′ junction sequence between flanking cotton genomic DNA and the transgenic insert of cotton event Gh_CSM63718, a 3′ junction sequence between flanking cotton genomic DNA and the transgenic insert of cotton event Gh_CSM63718, SEQ ID NO: 9, and a fragment of SEQ ID NO: 9 comprising a sufficient length of contiguous nucleotides of SEQ ID NO: 9 to identify the sequence as a fragment of the transgenic insert of Gh_CSM63718. The presence of the DNA amplicon indicates the presence of cotton event Gh_CSM63718 in the sample. The DNA amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length. The DNA amplicon can comprise a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 17,736-17,737 of SEQ ID NO:10.

A further method of detecting the presence of cotton event Gh_CSM63718 in a sample of DNA derived from a cotton seed, plant, plant part, plant cell, progeny plant or commodity product is provided. The method comprises: (a) contacting the sample with any of the DNA molecules that function as a probe specific for cotton event Gh_CSM63718 described herein; and (b) performing a sequencing reaction to produce a target sequence. The target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 17,736-17,737 of SEQ ID NO:10.

Another method of detecting the presence of cotton event Gh_CSM63718 in a sample derived from a cotton seed, plant, plant part, cell, progeny plant or commodity product is provided. The method comprises: (a) contacting the sample with an antibody specific for the PPO protein encoded by cotton event Gh_CSM63718, an antibody specific for the TDO protein encoded by cotton event Gh_CSM63718, or a combination thereof; and (b) detecting binding of the antibody or antibodies to the protein or proteins in the sample. The binding of the antibody or antibodies indicates the presence of cotton event Gh_CSM63718 in the sample. The method can further comprise: (a) contacting the sample with an antibody specific for the DMO protein encoded by cotton event Gh_CSM63718, an antibody specific for the EPSPS protein encoded by cotton event Gh_CSM63718, an antibody specific for the PAT protein encoded by cotton event Gh_CSM63718, or a combination of any thereof; and (b) detecting binding of the antibody or antibodies to the protein or proteins in the sample. The binding of the antibody or antibodies indicates the presence of cotton event Gh_CSM63718 in the sample.

DNA detection kits for detecting the presence of cotton event Gh_CSM63718 in a sample are provided. One example of such a DNA detection kit is a kit comprising: (a) any of the pairs of DNA molecules that can be used to produce an amplicon diagnostic for cotton event Gh_CSM63718 described herein; and/or (b) any of the DNA molecules that function as a probe specific for cotton event Gh_CSM63718 described herein.

Also provided are protein detection kits for detecting the presence of cotton event Gh_CSM63718 in a sample. The kit comprises an antibody specific for the PPO protein encoded by cotton event Gh_CSM63718, an antibody specific for the TDO protein encoded by cotton event Gh_CSM63718, or a combination thereof. Detecting binding of the antibody or antibodies to the protein(s) encoded by cotton event Gh_CSM63718 in a sample is diagnostic for the presence of cotton event Gh_CSM63718 in the sample. The protein detection kit can further comprise an antibody specific for the DMO protein encoded by cotton event Gh_CSM63718, an antibody specific for the EPSPS protein encoded by cotton event Gh_CSM63718, an antibody specific for the PAT protein encoded by cotton event Gh_CSM63718, or a combination of any thereof.

Methods of determining the zygosity of a cotton plant, plant part, plant seed, or plant cell comprising cotton event Gh_CSM63718 are provided. One example of such a method comprises: (a) contacting a sample comprising DNA derived from the cotton plant, plant part, plant seed, or plant cell with a first primer set capable of producing a first amplicon diagnostic for the presence of cotton event Gh_CSM63718, and a second primer set capable of producing a second amplicon diagnostic for wildtype cotton genomic DNA not comprising cotton event Gh_CSM63718; (b) performing a nucleic acid amplification reaction; and (c) detecting the first amplicon and the second amplicon. The presence of both amplicons indicates that the plant, plant part, seed or cell is heterozygous for cotton event Gh_CSM63718. The presence of only the first amplicon indicates that the plant, plant part, seed, or cell is homozygous for cotton event Gh_CSM63718. For example, the first primer set can comprise SEQ ID NO:19 and SEQ ID NO:20, and the second primer set comprises SEQ ID NO:19 and SEQ ID NO:22.

Another method of determining the zygosity of a cotton plant, plant part, plant seed, or plant cell comprising cotton event Gh_CSM63718 is provided. The method comprises: (a) contacting a sample comprising DNA derived from the cotton plant, plant part, plant seed, or plant cell with a probe set comprising at least a first probe that specifically hybridizes to cotton event Gh_CSM63718, and at least a second probe that specifically hybridizes to cotton genomic DNA that was disrupted by insertion of the heterologous DNA of cotton event Gh_CSM63718 but does not hybridize to cotton event Gh_CSM63718; and (b) hybridizing the probe set with the sample under stringent hybridization conditions. Detecting hybridization of only the first probe under the hybridization conditions is diagnostic for a cotton plant, plant part, seed or plant cell homozygous for cotton event Gh_CSM63718. Detecting hybridization of both the first probe and the second probe under the hybridization conditions is diagnostic for a cotton plant, plant part, seed, or plant cell heterozygous for cotton event Gh_CSM63718. For example, the probe set can comprise SEQ ID NO:21 and SEQ ID NO:23.

DNA constructs are provided. One example of a DNA construct provided herein is a DNA construct comprising a first expression cassette, a second expression cassette, a third expression cassette, a fourth expression cassette, and a fifth expression cassette. The first expression cassette comprises in operable linkage: (i) a ribulose bisphosphate carboxylase/oxygenase (RuBisCO) activase gene promoter, and a leader sequence from Arabidopsis thaliana, (ii) a codon-optimized phosphinothricin N-acetyltransferase (PAT) coding sequence from Streptomyces viridochromogenes, and (iii) a 3′ UTR of a small heat shock protein (Hsp20) from Medicago truncatula. The second expression cassette comprises in operable linkage: (i) an enhancer from the strawberry vein banding virus (SVBV) fused to the promoter and 5′ UTR from a CAB1 (Chlorophyll A/B Binding Protein) gene from Cucumis melo, (ii) a codon-optimized triketone dioxygenase (TDO) coding sequence from Oryza sativa, and (iii) a 3′ UTR of a TMA7 (translation machinery associated 7) protein from Medicago truncatula. The third expression cassette comprises in operable linkage: (i) a polyubiquitin gene (UBQ10) promoter, a leader and an intron sequence from Arabidopsis thaliana, (ii) an N-terminal chloroplast transit peptide coding sequence of APG6 (Albino and Pale Green 6) from Arabidopsis thaliana fused to a codon-optimized dicamba monooxygenase (DMO) coding sequence from Stenotrophomonas maltophilia; and (iii) a 3′ UTR of an aluminum-induced Sali3-2 protein from Medicago truncatula. The fourth expression cassette comprises in operable linkage: (i) an enhancer of the 35S gene from Figwort Mosaic Virus (FMV), (ii) a promoter, a leader sequence, and an intron sequence of the elongation factor 1A gene (ELFla) from Arabidopsis thaliana, (iii) an N-terminal chloroplast transit peptide of granule bound starch synthase I from Triticum aestivum fused to a codon optimized 5-enolpyruvylshikimate-3-phosphate synthase gene (EPSPS) from Agrobacterium sp strain CP4, and (iv) a 3′ UTR of a ribulose 1,5-bisphosphate carboxylase small subunit E9 (rbcS-E9) gene from Pisum sativum. The fifth expression cassette comprises in operable linkage: (i) an enhancer derived from multiple enhancer sequences from Arabidopsis thaliana, (ii) a promoter sequence designed from multiple promoter sequences from Arabidopsis thaliana, (iii) an intron and 5′ UTR for a cytochrome C oxidase subunit VIa gene from Arabidopsis thaliana fused a 5′ UTR designed from multiple 5′ UTR sequences from Arabidopsis thaliana, (iv) an N-terminal chloroplast transit peptide coding sequence of APG6 (Albino and Pale Green 6) from Arabidopsis thaliana, with monocot codon usage, fused to the coding region of a protoporphyrinogen oxidase (PPO) gene from Enterobacter cloacae with codons optimized for cotton, and (v) a 3′ UTR from the fiber FbLate-2 gene from Gossypium barbadense. For example, the DNA construct can comprise SEQ ID NO:9. The DNA construct can further comprise at the 5′ and/or 3′ end of said construct: (a) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; and/or (b) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or SEQ ID NO:15.

Another DNA construct is provided. The DNA construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the full length of SEQ ID NO: 9. The DNA construct comprises at the 5′ and/or 3′ end of said construct (i) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15.

Any of the DNA constructs can comprise at the 5′ end of said construct one or more nucleotide sequences selected from SEQ ID NOs:58-127. Any of the DNA constructs can comprise at the 3′ end of said construct one or more nucleotide sequences selected from SEQ ID NOs:128-197.

Methods for controlling or preventing weed growth in an area are provided. One example of such a method comprises planting cotton comprising event Gh_CSM63718 in the area and applying an effective amount of at least one herbicide selected from the group consisting of glufosinate, a ß-triketone HPPD inhibitor, dicamba, glyphosate, a PPO herbicide, and any combination thereof, to control weeds in the area without injury to the cotton or with less than about 10% injury to the cotton. Applying the effective amount of at least one herbicide can comprise applying at least two or more herbicides selected from the group consisting of glufosinate, a β-triketone HPPD inhibitor, dicamba, glypohsate, a PPO herbicide, and any combination thereof over a growing season.

Methods for controlling volunteer cotton comprising cotton event Gh_CSM63718 in an area are provided. One example of such a method comprises applying an herbicidally effective amount of at least one herbicide other than glufosinate, a ß-triketone HPPD inhibitor, dicamba, glyphosate, or a PPO herbicide. The herbicide application prevents growth of cotton comprising cotton event Gh_CSM63718. The herbicide other than glufosinate, a ß-triketone HPPD inhibitor, dicamba, glyphosate, or a PPO herbicide can be selected from the group consisting of atrazine, topramezone, clopyralid, pyrithiobac, fluometuron, (3-(3,4-dichlorophenyl)-1,1-dimethylurea) (DCMU), 2,4-D, thidiazuron, dichlorprop-p 2-ethylhexyl ester, dichlorprop-p, trifloxysulfuron, paraquat, diquat, and combinations of any thereof.

Methods of obtaining a seed of a cotton plant or a cotton plant that is tolerant to glufosinate, a ß-triketone HPPD inhibitor, dicamba, glyphosate, a PPO herbicide, or any combination thereof are provided. One example of such a method comprises: (a) obtaining a population of progeny seed or plants grown therefrom, at least one of which comprises cotton event Gh_CSM63718; and (b) identifying at least a first progeny seed or plant grown therefrom that comprises cotton event Gh_CSM63718. Identifying the progeny seed or plant grown therefrom that comprises cotton event Gh_CSM63718 can comprise: (a) growing the progeny seed or plant to produce progeny plants; (b) treating the progeny plants with an effective amount of at least one herbicide selected from the group consisting of glufosinate, a ß-triketone HPPD inhibitor, dicamba, glyphosate, a PPO herbicide, and combinations of any thereof; and (c) selecting a progeny plant that is tolerant to the at least one herbicide selected from the group consisting of glufosinate, a ß-triketone HPPD inhibitor, dicamba, glyphosate, a PPO herbicide, and combinations of any thereof. Alternatively or in addition, identifying the progeny seed or plant grown therefrom that comprises cotton event Gh_CSM63718 can comprise detecting the presence of cotton event Gh_CSM63718 in a sample derived from the progeny seed or plant grown therefrom. Alternatively or in addition, identifying the progeny seed or plant grown therefrom that comprises cotton event Gh_CSM63718 can comprise detecting the presence of at least one protein encoded by cotton event Gh_CSM63718 in a sample derived from the progeny seed or plant grown therefrom.

Methods of improving tolerance to at least one herbicide selected from the group consisting of glufosinate, a 0-triketone HPPD inhibitor, dicamba, glyphosate, a PPO herbicide, and combinations of any thereof in a cotton plant are provided. One example of such a method comprises: (a) inserting any of the DNA constructs described herein into the genome of a cotton cell; (b) generating a cotton plant from the cotton cell; and c) selecting a cotton plant comprising the DNA construct. The selecting can comprise treating the cotton cell or plant with an effective amount of at least one herbicide selected from the group consisting of glufosinate, a 0-triketone HPPD inhibitor, dicamba, glyphosate, a PPO herbicide, and combinations of any thereof.

Cotton plants, plant seeds, plant parts, and plant cells comprising a recombinant DNA molecule are provided. The recombinant DNA molecule comprises a sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. The cotton plant, plant seed, plant part, or plant cell expresses at least one herbicide tolerance gene selected from the group consisting of phosphinothricin N-acetyltransferase (PAT), triketone dioxygenase (TDO), dicamba monooxygenase (DMO), 5-enolpyruvylshikimate-3-phosphate synthase (EPSPS), protoporphyrinogen oxidase (PPO), and any combination thereof. The cotton plant, plant seed, plant part, or plant cell is tolerant to at least one herbicide selected from the group consisting of glufosinate, 0-triketone HPPD inhibitors, dicamba, glyphosate, PPO herbicides, and combinations of any thereof.

Further cotton plants, plant seeds, plant parts, and plant cells are provided. The cotton plants, plant seeds, plant parts, and plant cells are tolerant to at least one herbicide selected from the group consisting of glufosinate, ß-triketone HPPD inhibitors, dicamba, glyphosate, PPO herbicides, and combinations of any thereof. The cotton plants, plant seeds, plant parts, or plant cells comprise any of the DNA constructs described herein.

Any of the cotton plants, plant seeds, plant parts, or plant cells can comprise cotton event Gh_CSM63718, a representative sample of seed comprising the event having been deposited under ATCC Accession No. PTA-127638.

Any of the cotton plants, plant seeds, plant parts, or plant cells can be further defined as a progeny plant of any generation of a cotton plant comprising cotton event Gh_CSM63718, or a cotton plant part, plant seed, or plant cell derived therefrom.

A further cotton plant, plant part, plant seed, or plant cell is provided. The cotton plant, plant part, plant seed, or plant cell comprises cotton event Gh_CSM63718, a representative sample of seed comprising cotton event Gh_CSM63718 having been deposited under ATCC Accession No. PTA-127638.

The cotton plant part can comprise a microspore, pollen, an anther, an ovule, an ovary, a boll, a flower, an embryo, a stem, a bud, a node, a leaf, a root, or a callus.

Any of the cotton plants, plant seeds, plant parts, or plant cells can be obtained by any of the methods of obtaining a seed of a cotton plant or a cotton plant, or any of the methods of improving tolerance to at least one herbicide described herein.

A further cotton plant, plant part, plant seed, or plant cell is provided. The cotton plant, plant cell, plant part, or plant seed comprises a recombinant DNA construct integrated in chromosome 21. The recombinant DNA construct confers tolerance to at least one herbicide selected from the group consisting of glufosinate, a ß-triketone HPPD inhibitor, dicamba, glyphosate, a PPO herbicide, and combinations of any thereof. The recombinant DNA construct is integrated in a position of said chromosome flanked by at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15. The at least 50 contiguous nucleotides of SEQ ID NO:11 or SEQ ID NO:14 can comprise one or more nucleotide sequences selected from SEQ ID NOs:58-127. The at least 50 contiguous nucleotides of SEQ ID NO:12 or SEQ ID NO:15 can comprise one or more nucleotide sequences selected from SEQ ID NOs:128-197.

With respect to any of the cotton plants, plant cells, plant parts, and plant seeds, and any of the methods for controlling or preventing weed growth in an area, any of the methods of obtaining a seed of a soybean plant or a soybean plant, and any of the methods of improving tolerance to at least one herbicide, the ß-triketone HPPD inhibitor can be selected from the group consisting of mesotrione, benzobicyclon (BBC), tembotrione, sulcotrione, tefuryltrione, and combinations of any thereof. The PPO herbicide can be selected from the group consisting of diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof. The diphenylether can be selected from the group consisting of acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof. The N-phenylphthalimide can be selected from the group consisting of cinidon-ethyl, flumiclorac, flumiclorac-pentyl, and flumioxazin. The oxadiazole can be selected from the group consisting of oxadiargyl and oxadiazon. The oxazolidinedione can be pentoxazone. The phenylpyrazole can be selected from the group consisting of fluazolate, pyraflufen, and pyraflufen-ethyl. The pyrimidinedione can be selected from the group consisting of benzfendizone, butafenacil, epyrifencacil (S-3100), flupropacil, flufenoximacil, saflufenacil, and tiafenacil. The thiadiazole can be selected from the group consisting of fluthiacet-methyl and thidiazimin. The triazolinone can be selected from the group consisting of azafenidin, bencarbazone, carfentrazone, its salts and esters, and sulfentrazone. The benzoxazinone derivative can be 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)). The other PPO herbicide can be selected from the group consisting of chlorphthalim, flufenpyr, flufenpyr-ethyl, flumipropyn, pyraclonil, profluazol, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate; cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate; methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate (flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy} propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, 2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, {[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid, 2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, ethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetic acid, ethyl (2-{2-chloro-5-[4-(1,1-difluoroethyl)-3-methyl-2,6-dioxo-3,6-dihydropyrimidin-1(2H)-yl]-4-fluorophenoxy}phenoxy)acetate, (2-{2-chloro-5-[4-(1,1-difluoroethyl)-3-methyl-2,6-dioxo-3,6-dihydropyrimidin-1(2H)-yl]-4-fluorophenoxy}phenoxy)acetic acid, ethyl (2-{2-chloro-4-fluoro-5-[4-(1-fluoroethyl)-3-methyl-2,6-dioxo-3,6-dihydropyrimidin-1(2H)-yl]phenoxy}phenoxy)acetate, 2-methoxyethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, tetrahydrofuran-2-ylmethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, cyanomethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, methyl (2-{2-chloro-5-[4-(1,1-difluoroethyl)-3-methyl-2,6-dioxo-3,6-dihydropyrimidin-1(2H)-yl]-4-fluorophenoxy}phenoxy)(methoxy)acetate, methyl (2-{2-bromo-5-[4-(1,1-difluoroethyl)-3-methyl-2,6-dioxo-3,6-dihydropyrimidin-1(2H)-yl]-4-fluorophenoxy}phenoxy)(methoxy)acetate, [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetic acid, ethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, 2-methoxyethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, tetrahydrofuran-2-ylmethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, ethyl 2-[[3-[5-[4-(1,1-difluoroethyl)-3-methyl-2,6-dioxo-pyrimidin-1-yl]-4-fluoro-2-nitro-phenoxy]-2-pyridyl]oxy]acetate, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, and ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate.

In any of the methods involving the use of an effective amount of glufosinate, the effective amount of glufosinate can be about 0.4 lb/acre to about 1.6 lb/acre over a growing season. In any of the methods involving the use of an effective amount of a, a ß-triketone HPPD inhibitor, the ß-triketone HPPD inhibitor can comprise mesotrione and the effective amount of mesotrione can be about 0.09 lb/acre to about 0.36 lb/acre. In any of the methods involving the use of an effective amount of dicamba, the effective amount of dicamba can be about 0.5 lb/acre to about 2 lb/acre over a growing season. In any of the methods involving the use of an effective amount of glyphosate, the effective amount of glyphosate can be about 0.5 lb/acre to about 2.5 lb/acre over a growing season. In any of the methods involving the use of an effective amount of a PPO herbicide, the effective amount of the PPO herbicide can be about 0.0009 lb/acre to about 1.5 lb/acre over a growing season.

A method of producing a progeny cotton plant comprising cotton event Gh_CSM63718 is provided. The method comprises: (a) sexually crossing a first cotton plant that comprises cotton event Gh_CSM63718 with itself or a second cotton plant; (b) collecting one or more seeds produced from the cross; (c) growing one or more seeds to produce one or more progeny plants; and (d) selecting at least a first progeny plant or seed comprising cotton event Gh_CSM63718. Inbred or hybrid cotton plants and seeds comprising cotton event Gh_CSM63718 that are produced by this method are also provided herein.

A nonliving or nonregenerable cotton plant material is provided. The nonliving or nonregenerable cotton plant material comprises any of the recombinant DNA molecules provided herein or any of the DNA constructs provided herein.

Another nonliving or nonregenerable cotton plant material is provided. The nonliving or nonregenerable cotton plant material comprises cotton event Gh_CSM63718, a representative sample of seed comprising the cotton event cotton event Gh_CSM63718 having been deposited under ATCC Accession No. PTA-127638.

A commodity product is provided. The commodity product comprises any of the recombinant DNA molecules provided herein or any of the DNA constructs provided herein. The commodity product can be produced from a transgenic cotton plant, plant part, plant seed, or plant cell comprising cotton event Gh_CSM63718. The commodity product can comprise whole or processed seeds; viable or nonviable seeds; viable plant parts (such as roots, nodes, bolls, buds or leaves); viable plant cells; processed plant parts; processed plant tissues; dehydrated plant tissues; dehydrated plant parts; frozen plant tissues; frozen plant parts; food for human consumption such as cottonseed oil; plant parts processed for animal feed such as cottonseed meal and cottonseed hulls; cotton fiber; or cotton linters.

A method of producing a commodity product is provided. The method comprises: (a) obtaining a transgenic cotton plant, plant part, or plant seed comprising cotton event Gh_CSM63718; and (b) producing a commodity product from the transgenic cotton plant, plant part, or plant seed.

A method of controlling, preventing, or reducing the development of herbicide-tolerant weeds is provided. The method comprises cultivating in a crop growing environment a cotton plant comprising transgenes that provide tolerance to glufosinate, ß-triketone HPPD inhibitor herbicides, dicamba, glyphosate, and PPO herbicides.

Another method for controlling, preventing, or reducing the development of herbicide-tolerant weeds is provided. The method comprises: (a) cultivating in a crop growing environment a cotton plant comprising any of the DNA constructs described herein or event Gh_CSM63718; and (b) applying to the crop growing environment at least one herbicide selected from the group consisting of glufosinate, a ß-triketone HPPD inhibitor, dicamba, glyphosate, a PPO herbicide, and any combination thereof, wherein the cotton plant is tolerant to the at least one herbicide.

In any of the methods for controlling, preventing, or reducing the development of herbicide-tolerant weeds, the transgenes that provide tolerance to the herbicides can be present at a single genomic location in the cotton plant.

A method of reducing loci for cotton breeding is provided. The method comprises inserting a construct comprising transgenes that provide tolerance to glufosinate, ß-triketone HPPD inhibitor herbicides, dicamba, glyphosate and PPO herbicides as a single locus at a genomic location in a cotton plant.

Any of the cotton plants, plant cells, plant parts, or plant seeds, any of the nonliving or nonregenerable cotton plant materials, any of the commodity products, and in any of the methods described herein, the cotton plant, plant cell, plant part, plant seed, nonliving or nonregenerable cotton plant material, or commodity product can further comprises one or more transgenes for providing resistance to insect infestations. The transgenes for providing resistance to insect infestations can be selected from the group consisting of Cry1B.3, Cry1 Da_7, Vip3Cb1.1, Cry2Ab2, and combinations of any thereof. The Cry1B.3 transgene can comprise a polynucleotide sequence encoding a protein having the amino acid sequence of SEQ ID NO:225. The Cry1 Da_7 transgene can comprise a polynucleotide sequence encoding a protein having the amino acid sequence of SEQ ID NO:227. The Vip3Cb1.1 transgene can comprise a polynucleotide sequence encoding a protein having the amino acid sequence of SEQ ID NO:229. The Cry2Ab2 transgene can comprise a polynucleotide sequence encoding a protein having the amino acid sequence of SEQ ID NO:231. The cotton plant, plant cell, plant part, plant seed, nonliving or nonregenerable plant material, or commodity product can further comprise cotton event Gh_BCS246002 and/or cotton event MON15947. The transgenes for providing resistance to insect infestations can provide resistance to infestations by Lepidopteran pests selected from the group consisting of Cotton Bollworm (Helicoverpa zea), Tobacco Budworm (Heliothis virescens), Fall Armyworm (Spodoptera frugiperda), Old World Bollworm (Helicoverpa armigera), and combinations of any thereof. The transgenes in Gh_BCS246002 for providing resistance to insect infestations are present at a single genomic location in the cotton plant. The cotton plant, plant cell, plant part, seed can further comprises a recombinant DNA molecule comprising a sequence selected from the group consisting of SEQ ID NO:212; SEQ ID NO:213; SEQ ID NO:214; SEQ ID NO:215; SEQ ID NO:216; SEQ ID NO:217; SEQ ID NO:218; SEQ ID NO:219; SEQ ID NO:220; SEQ ID NO:221; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:212 or the full length of SEQ ID NO: 213; and a complete complement of any of the foregoing.

Further cotton plants, plant cells, plant parts, seeds, nonliving or nonregenerable cotton plant materials, and cotton commodity products are provided. The cotton plants, plant cells, plant parts, seeds, nonliving or nonregenerable cotton plant materials, and cotton commodity products comprise a foreign DNA at an insertion site in the cotton genome. The insertion site has a nucleic acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the sequence of SEQ ID NO:13. The foreign DNA can be flanked by 5′ and 3′ flanking regions, wherein said 5′ flanking region is upstream of and contiguous with said foreign DNA and comprises SEQ ID NO:11, and wherein said 3′ flanking region is downstream of and contiguous with said foreign DNA and comprises SEQ ID NO:12. The foreign DNA can comprise at least one herbicide tolerance gene selected from the group consisting of a phosphinothricin N-acetyltransferase (PAT) gene, a triketone dioxygenase (TDO) gene, a dicamba monooxygenase (DMO) gene, a 5-enolpyruvylshikimate-3-phosphate synthase (EPSPS) gene, a protoporphyrinogen oxidase (PPO) gene, and any combination of any thereof. For example, the foreign DNA can comprise a phosphinothricin N-acetyltransferase (PAT) gene, a triketone dioxygenase (TDO) gene, a dicamba monooxygenase (DMO) gene, a 5-enolpyruvylshikimate-3-phosphate synthase (EPSPS) gene, and a protoporphyrinogen oxidase (PPO) gene. The foreign DNA can comprise SEQ ID NO: 9. Alternatively, the foreign DNA can comprise SEQ SEQ ID NO:9 with one or more modifications. The one or more modifications can comprise one or more insertions, one or more deletions, one or more substitutions, or a combination of any thereof, within SEQ ID NO:9. For example, the one or more modifications can comprise a deletion of all or a portion of the phosphinothricin N-acetyltransferase (PAT) gene, a deletion of all or a portion of the triketone dioxygenase (TDO) gene, a deletion of all or a portion of the dicamba monooxygenase (DMO) gene, a deletion of all or a portion of the 5-enolpyruvylshikimate-3-phosphate synthase (EPSPS) gene, a deletion of all or a portion of the protoporphyrinogen oxidase (PPO) gene, or a combination of any thereof. Alternatively or in addition, the one or more modifications can comprise an insertion of an additional expression cassette.

A method of producing a cotton plant or seed is provided. The method comprises inserting foreign DNA at an insertion site in the genome of the cotton plant or seed. The insertion site has a nucleic acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the sequence of SEQ ID NO:13. The cotton plant produced by the method can have improved trait efficacy, increased expression of one or more transgenes, improved stability of one or more transgenes, improved agronomic characteristics, or any combination thereof, as compared to a plant that has the same foreign DNA inserted at a different insertion site in the genome. Cotton plants and seeds produced by the method are also provided.

Another method of producing a cotton plant or seed is provided. The method comprises obtaining a cotton plant or seed comprising cotton event Gh_CSM63718, a representative sample of seed comprising cotton event Gh_CSM63718 having been deposited under ATCC Accession No. PTA-127638, and inserting foreign DNA into the sequence of SEQ ID NO:10 of the plant or seed. Cotton plants and seeds produced by the method are also provided.

Yet another method of producing a cotton plant or seed is provided. The method method comprises obtaining a cotton plant or seed comprising event Gh_CSM63718, a representative sample of seed comprising cotton event Gh_CSM63718 having been deposited under ATCC Accession No. PTA-127638, and deleting all or a portion of the sequence of SEQ ID NO:10 in the plant or seed. Cotton plants and seeds produced by the method are also provided.

A method of detecting the presence a nucleic acid molecule encoding PPO_H_N90 in a sample derived from a cotton seed, plant, plant part or plant cell, progeny plant, or commodity product is provided. The method comprises: (a) contacting the sample with a pair of DNA molecules; (b) performing an amplification reaction sufficient to produce a DNA amplicon comprising the nucleic acid encoding the PPO_H_N90 or a portion of the nucleic acid encoding the PPO_H_N90 of sufficient length to identify the presence of the nucleic acid encoding the PPO_H_N90; and (c) detecting the presence of the DNA amplicon. The presence of the DNA amplicon indicates the presence of PPO_H_N90 in the sample. The pair of DNA molecules can comprise SEQ ID NO:234 and SEQ ID NO:235.

Another method of detecting the presence of a nucleic acid molecule encoding PPO_H_N90 in a sample derived from a cotton seed, plant, plant part, plant cell, progeny plant, or commodity product is provided. The method comprises: (a) contacting the sample with a DNA molecule that functions as a DNA probe specific for the nucleic acid molecule encoding PPO_H_N90; (b) subjecting the sample and the DNA molecule that functions as a probe to stringent hybridization conditions; and (c) detecting the hybridization of the DNA molecule that functions as a probe to a DNA molecule in the sample. The hybridization of the DNA molecule that functions as a probe to the DNA molecule in the sample is diagnostic for the presence of PPO_H_N90 in the sample. The DNA molecule that functions as a probe can comprise SEQ ID NO:236.

### BRIEF DESCRIPTION OF THE DRAWINGS

FIG. 1 illustrates the sequence of the cotton event Gh_CSM63718. Horizontal lines correspond to the positions of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:11, and SEQ ID NO:12 relative to SEQ ID NO:10. The horizontal arrows (SEQ ID NO:19 and SEQ ID NO:20) represent the approximate positions of an illustrative primer pair that can be used to detect cotton event Gh_CSM63718. The horizontal line labeled SEQ ID NO:21 represents the approximate position of an illustrative DNA probe that can be used to detect cotton event Gh_CSM63718. “RB” refers to the Agrobacterium T-DNA right border; “LB” refers to the Agrobacterium T-DNA left border. “PAT” represents a phosphinothricin N-acetyltransferase expression cassette; “TDO” represents a triketone dioxygenase expression cassette; “DMO” represents a dicamba monooxygenase expression cassette; “CP4” represents a 5-enolpyruvylshikimate-3-phosphate synthase expression cassette; “PPO” represents a protoporphyrinogen oxidase expression cassette; “Lox” represents a lox recombination site; and “ISR” represents a non-coding intergenic sequence designed to minimize potential effects of neighboring genes on each others' expression. The horizontal line labeled SEQ ID NO:13 represents the relative location or position of the wildtype cotton genome where the transgenes (SEQ ID NO:9) were inserted. The dashed line represents a 103-nucleotide deletion in the cotton genome at the site of insertion.

FIG. 2 is a diagrammatic representation of the T-DNA cassettes in the Agrobacterium Ti plasmid used to transform and generate cotton event Gh_CSM63718 before and after T-DNA integration, and after Cre-mediated excision of the marker and Cre cassettes. “RB” refers to the Agrobacterium T-DNA right border; “LB” refers to the Agrobacterium T-DNA left border. “aadA”, “Cre”, and “ISR1” and “ISR2” represent the aadA selectable marker cassette, the Cre recombinase cassette, and non-coding intergenic sequences, respectively. “PAT” represents a phosphinothricin N-acetyltransferase expression cassette; “TDO” represents a triketone dioxygenase expression cassette; “DMO” represents a dicamba monooxygenase expression cassette; “CP4” represents 5-enolpyruvylshikimate-3-phosphate synthase expression cassette; “PPO” represents a protoporphyrinogen oxidase expression cassette. “Lox” represents a lox recombination site. “5′ Flank” and “3′ Flank” represent the 5′ and 3′ flanking cotton genomic sequences at the site of T-DNA integration, respectively. A: “T-DNA Before Integration” represents T-DNA comprising the aadA and Cre cassettes, the ISR1, the PAT, TDO, DMO, and CP4 cassettes, the ISR2, and the PPO cassette before transformation; B: “Inserted T-DNA After Integration” represents T-DNA comprising the aadA and Cre cassettes, the ISR1, the PAT, TDO, DMO, and CP4 cassettes, the ISR2, and the PPO cassette integrated into the cotton genome after transformation; C: “Inserted T-DNA After Cre-Excision” represents the integrated T-DNA cassettes after the aadA, Cre, and ISR1 were excised, leaving behind one of the two Lox sites, the PAT, TDO, DMO, CP4 and PPO cassettes, ISR2, and the left and right borders.

FIG. 3 illustrates the approximate timelines for the research, testing, and development, leading to selection and identification of the commercial cotton event Gh_CSM63718. “POC” stands for Proof of Concept; “TFN” strands for Transformation; “GH” stands for Greenhouse; “NA” stands for North America.

FIG. 4 is a diagrammatic representation of the breeding process to produce marker-free cotton events. “R0 Transformants” are the initial transgenic events generated by transformation with the transformation vector, which were hemizygous for the T-DNA allele comprising the aadA, and the Cre cassette, and an intergenic sequence region (ISR1) flanked by two lox recombination sites, followed by the PAT, TDO, DMO, CP4, ISR2 and PPO cassettes. The R0 transformants were self-pollinated to produce the R1 generation seeds, wherein many of the progeny lost the aadA and the Cre cassettes, and ISR1 flanked by the two Lox sites due to Cre-recombinase excision. Hemizygous, T-DNA positive, aadA, Cre, and ISR1 negative (also referred to as “marker-free”) plants were selected and self-pollinated, resulting in an R2 generation. R2 plants homozygous for the inserted T-DNA allele without the aadA, CRE, and ISR1 were selected and self-pollinated to produce a pure line of R3 Gold Standard Seed (GSS). The process continued to produce subsequent generations for generation stability studies and for segregation studies and for trait integration into other germplasms. The R2, R3, and R4 generation plants which were marker-free and homozygous for the inserted T-DNA were used for efficacy and agronomic field trials. Subsequent “R” generations (R1, R2, R3 and so on) represent successive generations produced through self-pollination of plants derived from the initial R0 transformant that resulted in the cotton event Gh_CSM63718.

### BRIEF DESCRIPTION OF THE SEQUENCES

SEQ ID NO:1 is a 30-nucleotide sequence representing the 5′ junction region of the cotton genomic DNA and the integrated transgene insert. SEQ ID NO:1 corresponds to nucleotide positions 986-1,015 of SEQ ID NO:10.

SEQ ID NO:2 is a 30-nucleotide sequence representing the 3′ junction region of the integrated transgene insert and the cotton genomic DNA. SEQ ID NO:2 corresponds to nucleotide positions 17,722-17,751 of SEQ ID NO:10.

SEQ ID NO:3 is a 60-nucleotide sequence representing the 5′ junction region of the cotton genomic DNA and the integrated transgene insert. SEQ ID NO:3 corresponds to nucleotide positions 971-1,030 of SEQ ID NO:10.

SEQ ID NO:4 is a 60-nucleotide sequence representing the 3′ junction region of the integrated transgene insert and the cotton genomic DNA. SEQ ID NO:4 corresponds to nucleotide positions 17,707-17,766 of SEQ ID NO:10.

SEQ ID NO:5 is a 100-nucleotide sequence representing the 5′ junction region of the cotton genomic DNA and the integrated transgene insert. SEQ ID NO:5 corresponds to nucleotide positions 951-1,050 of SEQ ID NO:10.

SEQ ID NO:6 is a 100-nucleotide sequence representing the 3′ junction region of the integrated transgene insert and the cotton genomic DNA. SEQ ID NO:6 corresponds to nucleotide positions 17,687-17,786 of SEQ ID NO:10.

SEQ ID NO:7 is a 1,050-nucleotide sequence representing the 5′ genomic flank region of the cotton genomic DNA and 50 bp of the integrated transgene insert. SEQ ID NO:7 corresponds to nucleotide positions 1-1,050 of SEQ ID NO:10.

SEQ ID NO:8 is a 1,050-nucleotide sequence representing 50 bp of the 3′ junction region of the integrated transgene insert and the 3′ genomic flank region of the cotton genomic DNA. SEQ ID NO:8 corresponds to nucleotide positions 17,687-18,736 of SEQ ID NO:10.

SEQ ID NO:9 is a 16,736-nucleotide sequence corresponding to the transgene insert of cotton event Gh_CSM63718. SEQ ID NO:9 corresponds to nucleotide positions 1,001-17,736 of SEQ ID NO:10.

SEQ ID NO:10 is a 18,736-nucleotide sequence corresponding to the contig nucleotide sequence of the 5′ cotton genomic DNA sequence (SEQ ID NO:11), three random nucleotides inserted during T-DNA integration, the transgene insert in event Gh_CSM63718 (SEQ ID NO:9), two nucleotides inserted during T-DNA integration, and the 3′ cotton genomic DNA sequence (SEQ ID NO:12).

SEQ ID NO:11 is a 1,000-nucleotide sequence representing the 5′ flanking cotton genomic DNA up to the transgene insert (SEQ ID NO:9). SEQ ID NO:11 corresponds to nucleotide positions 1-1,000 of SEQ ID NO:10.

SEQ ID NO:12 is a 1,000-nucleotide sequence representing the 3′ flanking cotton genomic DNA after the transgene insert (SEQ ID NO:9). SEQ ID NO:12 corresponds to nucleotide positions 17,737-18,736 of SEQ ID NO:10.

SEQ ID NO:13 is a 2,103-nucleotide sequence representing wildtype cotton genomic DNA at the location where the transgenic sequence (SEQ ID NO:9) was inserted in event Gh_CSM63718. A 103-nucleotide fragment of SEQ ID NO:13 (nucleotides 1,001-1,103) was deleted in event Gh_CSM63718 due to insertion of the T-DNA.

SEQ ID NO:14 is a 5,000-nucleotide sequence representing cotton genomic DNA that flanks the transgenic insert at the 5′ end of the insert. The sequence is based on the genomic sequence of cotton™-1 germplasm.

SEQ ID NO:15 is a 5,000-nucleotide sequence representing cotton genomic DNA that flanks the transgenic insert at the 3′ end of the insert. The sequence is based on the genomic sequence of cotton™-1 germplasm.

SEQ ID NO:16 is a 23-nucleotide sequence corresponding to a thermal amplification primer referred to as SQ22496 used as an internal control for the event assay for cotton event Gh_CSM63718 and hybridizes to a region of the cotton genome.

SEQ ID NO:17 is a 19-nucleotide sequence corresponding to a thermal amplification primer referred to as SQ22497 used as an internal control for the event assay for cotton event Gh_CSM63718 and hybridizes to a region of the cotton genome.

SEQ ID NO:18 is a 14-nucleotide sequence corresponding to a VIC-MGB probe referred to as PB50562 used as an internal control for the event assay for cotton event Gh_CSM63718 and hybridizes to a region of the cotton genome.

SEQ ID NO:19 is a 24-nucleotide sequence corresponding to a thermal amplification primer referred to as SQ51787 used in event-specific assay and zygosity assay to detect cotton event Gh_CSM63718 DNA in a sample and is the reverse complement of the nucleotide sequence corresponding to positions 17,737-17,760 of SEQ ID NO:10.

SEQ ID NO:20 is a 22-nucleotide sequence corresponding to a thermal amplification primer referred to as SQ51702 used in event-specific assay and zygosity assay to detect cotton event Gh_CSM63718 DNA in a sample and is identical to the nucleotide sequence corresponding to positions 17,649-17,670 of SEQ ID NO:10.

SEQ ID NO:21 is an 18-nucleotide sequence corresponding to a 6FAM-MGB probe referred to as PB50308 used in event-specific assay and zygosity assay to detect cotton event Gh_CSM63718 DNA in a sample and is identical to the reverse complement of the nucleotide sequence corresponding to positions 17,676-17,693 of SEQ ID NO:10.

SEQ ID NO:22 is a 31-nucleotide sequence corresponding to a thermal amplification forward primer referred to as SQ52074 used in zygosity assay for detection of the wild-type (WT) allele DNA in a sample and hybridizes to a region of the cotton genome.

SEQ ID NO:23 is an 18-nucleotide sequence corresponding to a VIC-MGB probe referred to as PB50683 used in a zygosity assay for detection of the WT allele DNA in a sample and hybridizes to a region of the cotton genome.

SEQ ID NOs:24-57 are the nucleotide sequences for the genetic elements in the transgenic insert of cotton event Gh_CSM63718 and are further described in Table 1 hereinbelow.

SEQ ID NOs:58-127 are 50-nucleotide sequences in the 5′ flank genomic sequence of event Gh_CSM63718. SEQ ID NOs:58-77 are based on the genomic sequence of the transformation germplasm; SEQ ID NOs:78-127 are based on the genomic sequence of cotton™-1 germplasm.

SEQ ID NOs:128-197 are 50-nucleotide sequences in the 3′ flank genomic sequence of event Gh_CSM63718. SEQ ID NOs:128-147 are based on the genomic sequence of the transformation germplasm; SEQ ID NOs:148-197 are based on the genomic sequence of cotton™-1 germplasm.

SEQ ID NOs:198 and 199 are the nucleotide and amino acid sequences of LbCas12a (also known as LbCpf1) of Lachnospiraceae bacterium ND2006, respectively.

SEQ ID NO:200 is the amino acid sequence for LbCas12a_V1 (G532R/K595R).

SEQ ID NO:201 is the amino acid sequence for LbCas12a_V2 (G532R/K538V/Y542R).

SEQ ID NO:202 is the amino acid sequence for Cas12a of Francisella_novicida (FnCas12a).

SEQ ID NO:203 is the nucleotide sequence for the gRNA repeat for LbCas12a.

SEQ ID NO:204 is the nucleotide sequence for the gRNA repeat for FnCas12a.

SEQ ID NO:205 is the nucleotide sequence for the gRNA gRNA_5F-87.

SEQ ID NO:206 is the nucleotide sequence for the gRNA gRNA_3F-45.

SED ID NO:207 is the amino acid sequence for the PAT protein.

SED ID NO:208 is the amino acid sequence for the TDO protein.

SED ID NO:209 is the amino acid sequence for the DMO protein.

SED ID NO:210 is the amino acid sequence for the CP4 EPSPS protein.

SED ID NO:211 is the amino acid sequence for the PPO protein.

SEQ ID NO:212 is a 17,129-nucleotide sequence corresponding to the contig nucleotide sequence of the 5′ genomic flanking DNA nucleotide sequence, the inserted T-DNA nucleotide sequence in cotton event GH_BCS246002, and the 3′ genomic flanking DNA nucleotide sequence.

SEQ ID NO:213 is a 15,129-nucleotide sequence corresponding to the transgenic inserted T-DNA of cotton event GH_BCS246002.

SEQ ID NO:214 is a 50-nucleotide sequence representing the 5′ junction region of cotton genomic DNA and the integrated transgenic expression cassette in cotton event GH_BCS246002.

SEQ ID NO:215 is a 100-nucleotide sequence representing the 5′ junction region of cotton genomic DNA and the integrated transgenic expression cassette in cotton event GH_BCS246002.

SEQ ID NO:216 is a 200-nucleotide sequence representing the 5′ junction region of cotton genomic DNA and the integrated transgenic expression cassette in cotton event GH_BCS246002.

SEQ ID NO:217 is a 1,200-nucleotide sequence representing the 5′ junction region of cotton genomic DNA and the integrated transgenic expression cassette in cotton event GH_BCS246002.

SEQ ID NO:218 is a 50-nucleotide sequence representing the 3′ junction region of integrated transgenic expression cassette in cotton event GH_BCS246002.

SEQ ID NO:219 is a 100-nucleotide sequence representing the 3′ junction region of integrated transgenic expression cassette in cotton event GH_BCS246002.

SEQ ID NO:220 is a 200-nucleotide sequence representing the 3′ junction region of integrated transgenic expression cassette in cotton event GH_BCS246002.

SEQ ID NO:221 is a 1,200-nucleotide sequence representing the 3′ junction region of integrated transgenic expression cassette in cotton event GH_BCS246002.

SEQ ID NO:222 is a 1,000-nucleotide sequence representing the 5′ flanking cotton genomic DNA up to the inserted T-DNA in cotton event GH_BCS246002.

SEQ ID NO:223 is a 1,000-nucleotide sequence representing the 3′ flanking cotton genomic DNA after the inserted T-DNA in cotton event GH_BCS246002.

SEQ ID NOs:224 and 225 are the nucleotide and amino acid sequences of Cry1B.3, a chimeric insect toxin comprised of domains 1 and 2 of Cry1Be, domain 3 of Cry1Ka, and a protoxin domain of Cry1Be.

SEQ ID NOs:226 and 227 are the nucleotide and amino acid sequences of Cry1 Da_7, a Cry1 Da insect toxin with amino acid modifications to improve efficacy.

SEQ ID NOs:228 and 229 are the nucleotide and amino acid sequences of Vip3Cb1.1, a VipCb1 insect toxin.

SEQ ID NOs:230 and 231 are the nucleotide and amino acid sequences of Cry2Ab.

SEQ ID NO:232 is a 2,267-nucleotide sequence representing 1,877 nucleotides of the 5′ flank region of the cotton genomic DNA and 390 nucleotides of the integrated transgene insert in cotton event MON15947.

SEQ ID NO:233 is a 1,360-nucleotide sequence representing 349 nucleotides of the integrated transgene insert and 1,012 nucleotides of the 3′ flank region of the cotton genomic DNA in cotton event MON15947.

SEQ ID NO:234 is a 23-nucleotide sequence corresponding to a thermal amplification primer referred to as SQ51334 used in an element-specific assay to detect PPO_H_N90 DNA in a sample and is the reverse complement of the nucleotide sequence corresponding to positions 295-317 of SEQ ID NO:50.

SEQ ID NO:235 is a 19-nucleotide sequence corresponding to a thermal amplification primer referred to as SQ51335 used in an element-specific assay to detect PPO_H_N90 DNA in a sample and is identical to the nucleotide sequence corresponding to positions 174-192 of SEQ ID NO:50.

SEQ ID NO:236 is a 19-nucleotide sequence corresponding to a 6FAM-MGB probe referred to as PB50535 used in an element-specific assay to detect PPO_H_N90 DNA in a sample and is identical to the reverse complement of the nucleotide sequence corresponding to positions 319-337 of SEQ ID NO:50.

SEQ ID NO:237 is a 20-nucleotide sequence corresponding to a VIC-MGB probe referred to as PB13032 used as an internal control for the element-specific assay for detection of PPO_H_N90 and hybridizes to a region of the cotton genome.

### DETAILED DESCRIPTION

The following definitions, descriptions, and methods are provided to better define the invention and to guide those of ordinary skill in the art in the practice of the invention. Unless otherwise noted, terms are to be understood according to conventional usage by those of ordinary skill in the relevant art.

Herbicide tolerance is an important agronomic trait for effective weed control to maintain favorable crop growing conditions and crop yields and is achieved by engineering of herbicide tolerance transgenes in crop plants using modern plant biotechnology techniques. Cotton event Gh_CSM63718 confers tolerance to glufosinate, ß-triketone HPPD inhibitors, dicamba, glyphosate, PPO herbicides, and any combination thereof and provides multiple modes of action for weed control and herbicide-resistant weed management.

Cotton event Gh_CSM63718 is provided. The event Gh_CSM63718 was produced by Agrobacterium-mediated transformation of cotton seed-derived embryo explants with a DNA construct comprising seven transgene expression cassettes: 1) a PAT expression cassette for conferring tolerance to glufosinate, comprising in operable linkage i) a ribulose bisphosphate carboxylase/oxygenase (RuBisCO) activase gene promoter, and a leader sequence from Arabidopsis thaliana, ii) a codon-optimized phosphinothricin N-acetyltransferase (PAT) coding sequence from Streptomyces viridochromogenes, and iii) a 3′ untranslated region (UTR) of a small heat shock protein (Hsp20) from Medicago truncatula; 2) a TDO expression cassette for conferring tolerance to ß-triketone HPPD inhibitor herbicides, comprising in operable linkage i) an enhancer from the strawberry vein banding virus (SVBV) fused to the promoter and 5′ UTR from a chlorophyll A/B binding protein (CAB1) gene from Cucumis melo, ii) a codon-optimized triketone dioxygenase (TDO, also known as HISi) coding sequence from Oryza sativa, and iii) a 3′ UTR of a translation machinery associated 7 protein (TMA7) from Medicago truncatula; 3) a DMO expression cassette for conferring tolerance to dicamba, comprising in operable linkage i) a polyubiquitin gene (UBQ10) promoter, a leader and an intron sequence from Arabidopsis thaliana, ii) an N-terminal chloroplast transit peptide coding sequence of albino and pale green (APG6) from Arabidopsis thaliana fused to a codon-optimized dicamba monooxygenase coding sequence from Stenotrophomonas maltophilia; and iii) a 3′ UTR of an aluminum-induced Sali3-2 protein from Medicago truncatula; 4) an CP4 EPSPS (also known as CP4) expression cassette for conferring tolerance to glyphosate, comprising in operable linkage i) an enhancer of the 35S gene from Figwort Mosaic Virus (FMV), ii) a promoter, a leader sequence, and an intron sequence of the elongation factor 1A gene (ELF1a) from Arabidopsis thaliana, iii) an N-terminal chloroplast transit peptide of granule bound starch synthase I from Triticum aestivum fused to a codon optimized 5-enolpyruvylshikimate-3-phosphate synthase gene (EPSPS) from Agrobacterium sp. strain CP4, and iv) a 3′ UTR of a ribulose 1,5-bisphosphate carboxylase small subunit E9 (rbcS-E9) gene from Pisum sativum; 5) a PPO expression cassette for conferring tolerance to PPO herbicides, comprising in operable linkage i) an enhancer derived from multiple enhancer sequences from Arabidopsis thaliana, ii) a promoter sequence designed from multiple promoter sequences from Arabidopsis thaliana, iii) an intron and 5′ UTR for a cytochrome C oxidase subunit VIa gene from Arabidopsis thaliana fused a 5′ UTR designed from multiple 5′ UTR sequences from Arabidopsis thaliana, iv) an N-terminal chloroplast transit peptide coding sequence of APG6 from Arabidopsis thaliana, with monocot codon usage fused to the coding region of a protoporphyrinogen oxidase gene from Enterobacter cloacae with codons optimized for cotton, and v) a 3′ UTR from the fiber FbLate-2 gene from Gossypium barbadense; 6) an aadA expression cassette as a selectable marker for selection of transgenic events, comprising in operable linkage i) a promoter, 5 untranslated region and intron sequences of the actin 7 gene (Act7) from Arabidopsis thaliana, ii) an N-terminal chloroplast transit peptide from 5-enolpyruvylshikimate-3-phosphate synthase (EPSPS) from Arabidopsis thaliana, fused to the coding region of the Tn7 adenylyltransferase from Escherichia coli, and iii) a 3′ UTR of the nopaline synthase (NOS) gene from Agrobacterium tumefaciens; 7) a Cre expression cassette for removal of the aadA and Cre cassettes flanked by two Lox sites, comprising in operable linkage i) a promoter and 5′ UTR of an anther specific LAT52 gene from Solanum lycopersicum, ii) a coding region of CRE recombinase from Enterobacteria phage P1, inserted internally with the second intron from the light inducible gene (LS1) from Solanum tuberosum for minimized bacterial expression, iii) a 3′ UTR of a cell division cycle 45 (CDC45) gene from Arabidopsis thaliana.

Plant transformation techniques, such as Agrobacterium-mediated or biolistic transformation, can be used to insert foreign DNA (also known as transgenic DNA) randomly or by site directed insertion (also known as targeted insertion) into a chromosome in a plant cell to produce a genetically engineered plant cell, also referred to as a “transgenic” or “recombinant” cell. Using these transformation techniques, many individual cells can be transformed, each resulting in a unique “transgenic event” or “event” due to insertion of the foreign DNA into the genome. A transgenic plant can then be regenerated from each individual transgenic cell. This results in every cell of the transgenic plant containing the uniquely inserted transgenic event as a stable part of its genome. The transgenic plant can then be used to produce progeny plants, each containing the unique transgenic event. The term “transgenic” refers to a plant, plant part, plant cell, seed, progeny plant, or DNA molecule, construct, or sequence comprising a transgene—e.g., a “transgenic cell” refers to a cell comprising a transgene.

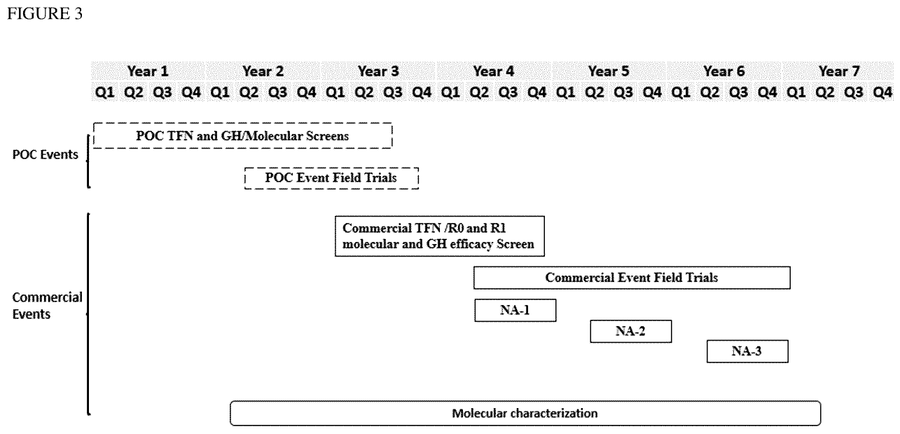

Cotton event Gh_CSM63718 was produced and identified by a complex research and development process. This process included: (i) design and selection of DNA constructs comprising the seven transgene cassettes based on design and testing of individual transgene cassettes with combinations of different expression elements; (ii) transformation of thousands of cotton cells with the DNA constructs; (iii) regeneration of a large population of transgenic events; and (iv) rigorous multi-year construct and event selection involving molecular characterization of the large number of transgenic events, greenhouse and field trials for herbicide tolerance efficacy and agronomic performance at different locations and in different geographies. Cotton event Gh_CSM63718 was thus produced and selected as a uniquely superior event useful for broad-scale agronomic commercial purposes. FIG. 3 illustrates the approximate timelines for the research, testing and development, leading to selection of the commercial event cotton event Gh_CSM63718.

Detailed molecular characterization was conducted on transgenic events. Event Gh_CSM63718 was selected based on stringent molecular criteria, as well as other selection criteria such as herbicide tolerance efficacy and agronomic performance. The results from such molecular analyses confirmed that: (1) event Gh_CSM63718 contains one copy of the inserted T-DNA at a single genomic location; (2) only the PAT, TDO, DMO, EPSPS and PPO expression cassettes and one lox site between the left and right borders of the T-DNA are present in the event, and no additional element from the transformation construct was present, such as the transformation construct backbone sequence or the aadA or Cre cassette, or the ISR1 or fragment(s); (3) the transgenic DNA was inserted in an intergenic region, far away from any endogenous genes or repeat regions; and (4) the transgenic event produced the correct sized transcripts and proteins for the PAT, TDO, DMO, EPSPS and PPO transgenes, verified by Northern hybridization and Western hybridization analyses, respectively. Furthermore, DNA sequence analyses and protein expression assays were performed to: (1) determine the 5′ and 3′ transgenic insert-to-plant genome junctions; (2) confirm the organization of the transgene cassettes and elements within the insert; (3) verify the complete nucleotide sequence of the inserted transgenic DNA (SEQ ID NO:9); and (4) determine the PAT, TDO, DMO, EPSPS and PPO protein levels in different tissues such as leaf and seed over multiple generations. In addition, primers and probes were designed, and thermal amplification assays were developed and verified for producing specific amplicons diagnostic for the presence of event Gh_CSM63718 in a sample. As used herein, the 5′ and 3′ designations in reference to the junction, direction and site of the transgenic event insertion is relative to the left border to right border direction of the inserted T-DNA, with the 5′ junction and genomic sequence being upstream of the left border and transgene, and the 3′ junction and genomic sequence being downstream of the right border and transgene.

As used herein, an “expression cassette” or “cassette” or “transgene cassette” is a recombinant DNA molecule or sequence comprising a combination of distinct elements for expressing an RNA and/or protein encoded by the coding sequence of a transgene in a transformed plant cell or transformed plant comprising the transgene. As provided herein, an “expression cassette” or “cassette” or “transgene cassette” includes one or more regulatory element(s) operably linked to a coding or transcribable DNA sequence. The regulatory elements can include a promoter, a leader, a 5′ untranslated region (5′ UTR), an intron and/or a 3′ untranslated region (3′ UTR). The “expression cassette” or “cassette” or “transgene cassette” is recombinant and heterologous with respect to the transformed plant cell genome, as well as to the combination of the different genetic elements. For purposes of the present disclosure, such an “expression cassette” or “cassette” or “transgene cassette” is a recombinant DNA molecule or sequence that encodes a protein for conferring tolerance to at least one class of herbicides as described herein. Table 1 provides a list of the genetic elements contained in the transgene cassettes in the transgenic insert (SEQ ID NO:9) of cotton event Gh_CSM63718.

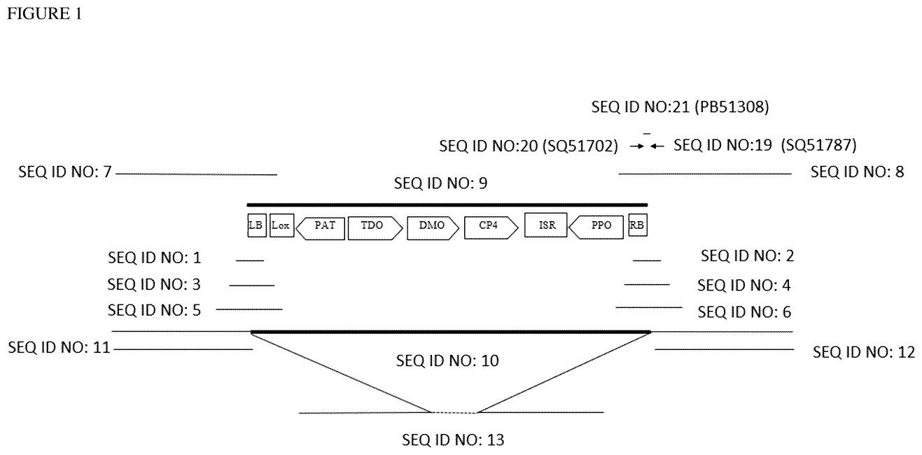

Insertion of the transgenic DNA into the genome of the cotton plant is accomplished by plant transformation methods known in the art and creates a new transgenic genomic DNA sequence, known as a “transgenic event” or an “event” or a “transgenic event locus.” The DNA sequence of the event consists of the inserted foreign DNA (referred to as the “transgenic insert”) and the genomic DNA adjacent to, or “flanking” the transgenic insert on either side of the insertion location. As used herein, the term “flanking” in reference to a transgenic event refers to the plant genomic sequence(s) adjacent to the transgenic DNA insertion in the genome of the transformed plant, plant part, plant tissue, or plant cell comprising the transgenic event on the 5′ and/or 3′ end(s) of the transgenic event insertion. Likewise, “flanking DNA” refers to a length of genomic DNA sequence adjacent to the transgenic DNA insertion in the genome of the transformed event on the 5′ and/or 3′ end(s) of the insertion. A “5′ flank”, therefore, means the cotton genomic DNA sequence adjacent to and upstream (or on the 5′ end) of the transgenic DNA insertion. For example, a “5′ flank” can include the cotton genomic DNA sequence immediately adjacent to and upstream (on the 5′ end) of the transgenic insertion, or any cotton genomic DNA sequence upstream (on the 5′ end) of the transgenic insertion that is not immediately adjacent to the transgenic insertion but is within about 5000 nucleotides, within about 3000 nucleotides, or within about 1000 nucleotides upstream of the transgenic insertion. Likewise, a “3′ flank” means the cotton genomic DNA sequence adjacent to and downstream (or on the 3′ end) of the transgenic insert. For example, a “3′ flank” can include the cotton genomic DNA sequence immediately adjacent to and downstream (on the 3′ end) of the transgenic insertion, or any cotton genomic DNA sequence downstream (on the 3′ end) of the transgenic insertion that is not immediately adjacent to the transgenic insertion but is within about 5000 nucleotides, within about 3000 nucleotides, or within about 1000 nucleotides downstream of the transgenic insertion. The DNA sequence of an event is unique to and specific for the event and can be readily identified when compared to other DNA sequences, such as that of other events or untransformed cotton genomic DNA. Cotton event Gh_CSM63718 has the new and unique DNA sequence provided as SEQ ID NO:10, which comprises a contiguous sequence comprising the 5′ cotton genomic flanking sequence provided as SEQ ID NO:11, the transgenic insert sequence provided as SEQ TD NO: 9, and the 3′ cotton genomic flanking sequence provided as SEQ ID NO:(12 (FIG. 1). Cotton event Gh_CSM63718 is thus a DNA molecule that is an integral part of the chromosome of transgenic cotton cells and plants comprising the event and as such is static and may be passed on to progeny cells and plants. As is described further in the Examples hereinbelow, various gene editing tools exist that would permit modification of the transgenic insert and/or the flanking genomic DNA of cotton event Gh_CSM63718, such as by deletion, insertion, transposition, or substitution of nucleic acid sequence(s); the event is still uniquely characterized by the presence of heterologous DNA at the particular position in the genome occupied by cotton event Gh_CSM63718 relative to flanking portions ofthe native cotton genome.

TABLE 1
Elements and Description of Cotton Event Gh_CSM63718

| | | Position in | |
|---|---|---|---|
| | SEQ | SEQ ID | |
| Element | ID NO | NO: 10 | Description |
| 5′ Flank sequence | 11 | 1-1000 | Cotton genomic DNA sequence flanking the 5′ |
| | | | end of the transgenic insert. |
| Left border region | 24 | 1001-1221 | Left border sequence from Agrobacterium |
| | | | tumefaciens. (SEQ ID NO: 24 is the full-length, |
| | | | 221-nucleotide T-DNA left border sequence. |
| | | | However, since a portion of this sequence was |
| | | | truncated at the time of the T-DNA insertion |
| | | | into the cotton genomic DNA, only nucleotides |
| | | | 1-221 of SEQ ID NO: 24 are present in SEQ ID |
| | | | NO: 10.) |
| Intervening sequence 1 | — | 1222-1231 | Sequence used in DNA cloning. |
| RS-P1.lox | 25 | 1232-1265 | Lox recombination site from Enterobacteria |
| | | | phage P1. |
| Intervening sequence 2 | — | 1266-1307 | Sequence used in DNA cloning. |
| T-Mt.Hsp20 | 26 | 1308-1807 | 3′ UTR for a small heat shock protein (Hsp20) |
| | | | from Medicago truncatula. |
| Intervening sequence 3 | — | 1808-1815 | Sequence used in DNA cloning. |
| CR-STRvi.Pat | 27 | 1816-2367 | Dicot codon-optimized phosphinothricin N- |
| | | | acetyltransferase (PAT) gene from Streptomyces |
| | | | viridochromogenes. |
| Intervening sequence 4 | — | 2368-2373 | Sequence used in DNA cloning. |
| L-At.Rca | 28 | 2374-2576 | 5′ UTR for the ribulose bisphosphate |
| | | | carboxylase/oxygenase (RuBisCO) activase |
| | | | gene from Arabidopsis thaliana. |
| P-At.Rca | 29 | 2577-3997 | Promoter for the ribulose bisphosphate |
| | | | carboxylase/oxygenase (RuBisCO) activase |
| | | | gene from Arabidopsis thaliana. |
| Intervening sequence 5 | — | 3998-4005 | Sequence used in DNA cloning. |
| E-SVBV.FLT | 30 | 4006-4309 | Enhancer from the strawberry vein banding |
| | | | virus (SVBV). |
| Intervening sequence 6 | — | 4310-4315 | Sequence used in DNA cloning. |
| P-CUCme.Cab1 | 31 | 4316-5401 | Promoter from a Chlorophyll A/B Binding |
| | | | Protein (CAB1) gene from Cucumis melo. |
| L-CUCme.Cab1 | 32 | 5402-5455 | 5′ UTR from a CAB1 gene from Cucumis melo. |
| Intervening sequence 7 | — | 5456-5463 | Sequence used in DNA cloning. |
| CR-Os.TDO | 33 | 5464-6519 | Codon-optimized sequence encoding triketone |
| | | | dioxygenase from Oryza sativa. |
| Intervening sequence 8 | — | 6520-6526 | Sequence used in DNA cloning. |
| T-Mt.AC140914v20 | 34 | 6527-7026 | 3′ UTR of a translation machinery associated 7 |
| | | | (TMA7) protein from Medicago truncatula. |
| Intervening sequence 9 | — | 7027-7034 | Sequence used in DNA cloning. |
| P-At.Ubq10 | 35 | 7035-7863 | Promoter from a polyubiquitin gene (UBQ10) |
| | | | from Arabidopsis thaliana. |
| L-At.Ubq10 | 36 | 7864-7930 | 5′ UTR of UBQ10 from Arabidopsis thaliana. |
| I-At.Ubq10 | 37 | 7931-8236 | Intron of UBQ10 from Arabidopsis thaliana. |
| TS-At.APG6 | 38 | 8237-8440 | Coding sequence of N-terminal chloroplast |
| | | | transit peptide from Albino and Pale Green 6 |
| | | | (APG6) from Arabidopsis thaliana. |
| CR-PSEma.DMO | 39 | 8441-9463 | Codon-optimized dicamba monooxygenase |
| | | | (DMO) coding sequence from |
| | | | Stenotrophomonas maltophilia. |
| T-Mt.Sali3 | 40 | 9464-9963 | 3′ UTR of the aluminum-induced Sali3-2 gene |
| | | | from Medicago truncatula. |
| Intervening sequence 10 | — | 9964-9971 | Sequence used in DNA cloning. |
| E-FMV.35S | 41 | 9972-10508 | Enhancer of the 35S gene from Figwort Mosaic |
| | | | Virus (FMV). |
| Intervening sequence 11 | — | 10509-10531 | Sequence used in DNA cloning. |
| P-At.Tsf1 | 42 | 10532-11011 | Promoter of the elongation factor 1A gene |
| | | | (ELF1a) from Arabidopsis thaliana. |
| L-At.Tsf1 | 43 | 11012-11057 | 5′ UTR of the ELF1a gene from Arabidopsis |
| | | | thaliana. |
| I-At.Tsf1 | 44 | 11058-11679 | Intron of the ELF1a gene from Arabidopsis |
| | | | thaliana. |
| Intervening sequence 12 | — | 11680-11688 | Sequence used in DNA cloning. |
| TS-Ta.waxy | 45 | 11689-11898 | Codon-optimized coding sequence of N- |
| | | | terminal chloroplast transit peptide of granule- |
| | | | bound starch synthase I from Triticum aestivum. |
| CR-AGRtu.aroA-CP4 | 46 | 11899-13266 | Codon-optimized 5-enolpyruvylshikimate-3- |
| | | | phosphate synthase (EPSPS) coding sequence |
| | | | from Agrobacterium sp. strain CP4. |
| Intervening sequence 13 | — | 13267-13283 | Sequence used in DNA cloning. |
| T-Ps.RbcS2-E9 | 47 | 13284-13926 | 3′ UTR of the ribulose 1,5-bisphosphate |
| | | | carboxylase small subunit E9 (rbcS-E9) gene |
| | | | from Pisum sativum. |
| Intervening sequence 14 | — | 13927-13934 | Sequence used in DNA cloning. |
| ISR2 | 48 | 13935-14958 | Non-coding, biologically neutral intergenic |
| | | | sequence. |
| Intervening sequence 15 | — | 14959-14966 | Sequence used in DNA cloning. |
| T-Gb.FbL2 | 49 | 14967-15281 | 3′ UTR of the fiber FbLate-2 gene from |
| | | | Gossypium barbadense. |
| CR-ENTcl.PPO_H_N90 | 50 | 15282-15821 | Codon-optimized coding sequence of a |
| | | | protoporphyrinogen oxidase (PPO) from |
| | | | Enterobacter cloacae. |
| TS-At.APG6-Mc | 51 | 15822-16025 | Coding sequence of N-terminal chloroplast |
| | | | transit peptide from APG6 from Arabidopsis |
| | | | thaliana. |
| Intervening sequence 16 | — | 16026-16028 | Sequence used in DNA cloning. |
| I-At.Cyco | 52 | 16029-16376 | Intron for a Cytochrome c oxidase subunit VIa |
| | | | gene from Arabidopsis thaliana. |
| L-At.Cyco | 53 | 16377-16448 | 5′ UTR of a Cytochrome c oxidase subunit VIa |
| | | | gene from Arabidopsis thaliana. |
| Intervening sequence 17 | — | 16449-16456 | Sequence used in DNA cloning. |
| L-At.GSP442 | 54 | 16457-16476 | 5′ UTR designed from multiple 5′ UTRs from |
| | | | Arabidopsis thaliana. |
| P-At.GSP442 | 55 | 16477-16956 | Promoter designed from multiple promoter |
| | | | sequences from Arabidopsis thaliana. |
| E-At.GSP571 | 56 | 16957-17378 | Enhancer derived from multiple enhancer |
| | | | sequences from Arabidopsis thaliana. |
| Intervening sequence 18 | — | 17379-17447 | Sequence used in DNA cloning. |
| B-AGRtu.right border | 57 | 17448-17736 | Right border sequence from Agrobacterium |
| | | | tumefaciens. (SEQ ID NO: 57 is the 289- |
| | | | nucleotide, full-length T-DNA right border |
| | | | sequence. A portion of this sequence was |
| | | | truncated during T-DNA insertion into the |
| | | | cotton genomic DNA. Only nucleotides 217- |
| | | | 505 of SEQ ID NO: 57 are present in SEQ ID |
| | | | NO: 10.) |
| 3′ Flank sequence | 12 | 17737-18736 | Cotton genomic DNA sequence flanking the 3′ |
| | | | end of the transgenic insert. |

Progeny of the original transformed cell and plant that comprise cotton event Gh_CSM63718 are provided. Such progeny may be produced by selfing of a cotton plant comprising cotton event Gh_CSM63718, or by sexual cross or outcrossing between a cotton plant comprising cotton event Gh_CSM63718 and another plant that does or does not contain the event, or by any other method known in the art including any plant cell or tissue culture method, wherein the progeny includes the cotton event Gh_CSM63718. The other plant may be a transgenic plant comprising the same and/or different event(s) or may be a non-transgenic plant, and each parental plant in a cross or outcross may be the same or different germplasm or breeding line. Cotton event Gh_CSM63718 is passed from the original parent through each generation to the progeny. A “transgenic plant” or “plant”, therefore, can be the original transformant plant regenerated from the transformed plant cell and comprising the transgenic DNA and event, or a progeny plant of the original transformant plant, which may be separated from the transformant by one or more generations, that retains the transgenic DNA and event at the same specific location and sequence context in the plant's genome. The transformant or progeny plant may be homozygous or heterozygous for event Gh_CSM63718. In addition, a “transgenic plant” may comprise a plant having the transgenes stably inserted into the genome of at least one cell of the plant (i.e., cotton event Gh_CSM63718 in at least one cell of the plant), and the plant may be chimeric or non-chimeric with respect to the transgenes and/or event. A transgenic plant is chimeric with respect to a transgene if not all cells of the plant comprise the transgenes.

The present disclosure describes introduction of event Gh_CSM63718 into cotton, and thus the term “cotton event Gh_CSM63718” is used to refer to the event herein. However, those of skill in the art will understand that event Gh_CSM63718 could be introduced into other varieties or related cotton species by crosses, such as Gossypium hirsutum (also known as upland cotton), Gossypium barbadense (also known as extra-long staple cotton), Gossypium arboretum (also known as tree cotton), Gossypium herbaceum (also known as levant cotton), and other Gossypium species.

Cotton event Gh_CSM63718 provides to cotton cells, plants, plant parts, seeds and progeny that comprise the event tolerance to inhibitors of glutamine synthetase such as glufosinate, ß-triketone HPPD inhibitors, benzoic acid auxin herbicides such as dicamba, EPSPS inhibitors such as glyphosate, PPO herbicides, and any combination thereof. The term “ß-triketone HPPD inhibitor(s)“and” ß-triketone HPPD inhibitor herbicide(s)” are used interchangeably herein and refer to chemical agents that target and inhibit the enzymatic activity of triketone dioxygenase. Cotton event Gh_CSM63718 provides tolerance to various “ß-triketone HPPD inhibitors, including, but not limited to, mesotrione, benzobicyclon (BBC), tembotrione, sulcotrione, tefuryltrione, and any combination thereof. The terms “PPO herbicide”, “PPO inhibitor”, and “PPO-inhibiting herbicide” are used interchangeably herein and refer to chemical agents that target and inhibit the enzymatic activity of a protoporphyrinogen oxidase (PPO). Cotton event Gh_CSM63718 provides tolerance to various PPO herbicides, including, but not limited to, flumioxazin, epyrifenacil (also referred to as S-3100 or rapidicil; IUPAC name: ethyl [(3-{2-chloro-5-[3,6-dihydro-3-methyl-2,6-dioxo-4-(trifluoromethyl)pyrimidin-1(2H)-yl]-4-fluorophenoxy}-2-pyridyl)oxy]acetate), lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate.

Cotton event Gh_CSM63718 is characterized as a single copy insertion into one locus in the cotton genome, resulting in two new loci or junction sequences (e.g., the sequences set forth in SEQ ID NO:1, SEQ ID NO:2, SEQ ID NO:3, SEQ ID NO:4, SEQ ID NO:5, SEQ ID NO:6, SEQ ID NO:7 and SEQ ID NO:8) spanning portions of the inserted DNA and the cotton genomic DNA that are not known to appear or exist naturally in the cotton genome or other transgenic cotton events, i.e., they are unique to event Gh_CSM63718. SEQ ID NO:1, SEQ ID NO:3, SEQ ID NO:5 and SEQ ID NO:7 span the 5′ junction of the cotton genomic sequence and the transgenic DNA insert, and SEQ ID NO:2, SEQ ID NO:4, SEQ ID NO:6 and SEQ ID NO:8 span the 3′ junction. These junction sequences are useful in detecting the presence of the event Gh_CSM63718 in cotton cells, seed, plants, plant parts, progeny, and plant products, such as cotton commodity products. Polynucleotide or DNA molecular probes and/or primer pairs are described herein for use in identifying the presence of these various junction sequences in biological samples containing or derived from, or suspected of containing or being derived from, cotton cells, seeds, plants, plant parts, progeny, or commodity products that contain the event Gh_CSM63718.

As used herein, the term “derived” or “derived from” in reference to a particular DNA molecule, amplicon or sequence in relation to a cotton plant, plant part, seed, progeny, cell and/or cotton plant product, such as a commodity product, means that the DNA molecule, amplicon or sequence is taken, purified, isolated, or made, directly or indirectly, from such cotton plant, plant part, seed, progeny, cell and/or cotton plant product, such as a commodity product. Alternatively, the term “derived” or “derived from” in reference to a cotton plant product, such as a commodity product, in relation to cotton plant, plant part, seed, progeny, or cell, means that the cotton plant product is taken, purified, isolated, or made, directly or indirectly, from such cotton plant, plant part, seed, progeny, or cell.

“Capable of being detected” refers to the ability of a particular DNA molecule, segment or sequence to be detected in a sample, such as by amplification and determining its presence, size or sequence such as by DNA sequence analysis, and/or binding of a probe to the target DNA molecule, segment or sequence.

A “sample” is intended to refer to any composition comprising or derived from, either directly or indirectly, a biological sample, source, or material. The sample may generally comprise cotton DNA and/or substantially or completely pure, purified, or isolated cotton DNA. A “biological sample” contains biological materials, including but not limited to DNA obtained or derived from, either directly or indirectly, the genome of a cotton cell(s), tissue(s), seed(s), plant(s), plant part(s) and/or cotton plant product(s), such as a commodity product(s). Such cotton cell(s), tissue(s), seed(s), plant(s), plant part(s) and/or cotton plant product(s), such as a commodity product(s), may comprise cotton event Gh_CSM63718, or DNA molecule(s) and/or DNA segment(s) comprising cotton event Gh_CSM63718. In some embodiments, a sample or biological sample may comprise cotton cell(s), cotton tissue(s), cotton seed(s), cotton plant(s), cotton plant part(s) and/or cotton plant product(s), whose cells or cellular membranes have been fractured (e.g., disrupted or opened) to release the contents of the cotton cell(s) including genomic DNA or proteins and/or make the contents of the cotton cell(s) including genomic DNA or proteins accessible or usable for assays or testing. “Directly” refers to directly obtaining DNA by a skilled artisan from the cotton genome by fracturing cotton cells (or by obtaining samples of cotton that contain fractured cotton cells) and exposing or using the genomic DNA or protein from cotton cells for the purposes of detection. “Indirectly” refers to obtaining by a skilled artisan a target or specific reference DNA (e.g., a novel and unique junction segment(s) described herein as being diagnostic for the presence of the event Gh_CSM63718) in a particular sample, by means other than by obtaining directly via fracturing of cotton cells or obtaining a sample of cotton that contains fractured cotton cells. Such indirect means include, but are not limited to, amplification of a DNA segment that contains a DNA sequence targeted by a particular probe(s) and/or primer set(s) designed to bind with specificity to or near the target sequence, or amplification of a DNA segment comprising all or part of a target sequence that can be measured and characterized (e.g., measured by migration or separation from other segments of DNA and/or identification in an effective matrix, such as an agarose or acrylamide gel or the like, or characterized by direct sequence analysis of the amplicon(s), or cloning of the amplicon(s) into a vector(s) and direct sequencing of the inserted amplicon(s) present within such vector(s)).

As used herein, the term “recombinant” refers to a non-naturally occurring DNA, protein, combination, or organism that would not normally be found or exist in nature and is created by human intervention. As used herein, a “recombinant DNA molecule” is a DNA molecule comprising a combination of DNA molecules that would not naturally occur together and is the result of human intervention. Two or more elements of such combination of DNA sequences may be operably linked to one another. For example, a recombinant DNA molecule may comprise a combination of at least two DNA molecules heterologous with respect to each other, such as a DNA molecule that comprises a coding sequence operably linked to a heterologous promoter and/or other regulatory expression element(s), and/or a transgene and a heterologous plant genomic DNA adjacent to the transgene, and/or a DNA molecule that is artificially synthesized and comprises a polynucleotide sequence that deviates from any polynucleotide sequence that would normally exist in nature. A recombinant DNA molecule may comprise all or part of a junction sequence of the genome of the event and all or part of the transgenic insert of the genome of the event, and/or may comprise a recombinant or heterologous DNA fragment of cotton event Gh_CSM63718. Examples of recombinant DNA molecules include a DNA molecule comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. Such recombinant DNA molecules can be derived from a cotton plant, seed, plant part, plant cell, progeny plant, or commodity product comprising cotton event Gh_CSM63718. Alternatively, such recombinant DNA molecules can be comprised in a cotton plant, seed, plant part, plant cell, or progeny plant comprising cotton event Gh_CSM63718, or a commodity product produced therefrom. A representative sample of seed comprising cotton event Gh_CSM63718 has been deposited as ATCC Accession No. PTA-127638. Such recombinant DNA molecules can be formed by the insertion of a heterologous nucleic acid molecule into the genomic DNA of a cotton plant or cotton cell. Such recombinant DNA molecules can be an amplicon diagnostic for the presence of cotton event Gh_CSM63718.

As used herein, a “recombinant” in reference to a plant, plant part, seed, plant cell, or progeny is a plant, plant part, seed, plant cell or progeny that would not normally exist in nature, is the result of human intervention, and contains a transgenic DNA molecule stably integrated into the genome of the plant, plant part, seed, plant cell, or progeny. As a result of such genomic insertion, the recombinant or transgenic plant, plant part, seed, plant cell, or progeny is something new and distinctly different from any related wildtype or naturally occurring plant, plant part, seed, plant cell or progeny. An example of a recombinant plant is a cotton plant containing the cotton event Gh_CSM63718.

As used herein, the term “transgene” refers to a DNA molecule artificially incorporated into an organism's genome as a result of human intervention, such as by plant transformation methods. A transgene may be heterologous to the organism. The term “transgenic insert” as used herein refers to the foreign or heterologous DNA inserted by plant transformation techniques into the cotton genome to produce cotton event Gh_CSM63718. The sequence for the transgenic insert of cotton event Gh_CSM63718 is provided as SEQ ID NO:9.

As used herein, the term “heterologous” in reference to a combination of two or more DNA sequences or elements means that the two or more DNA sequences or elements do not normally exist together as such combination in nature without human intervention. For example, a DNA molecule may be from a first species or a recombinant DNA molecule and inserted into the genome of a second species. The DNA molecule would thus be heterologous to the genome and the organism. As used herein, the term “heterologous” in reference to a DNA molecule, construct, sequence or protein in relation to a plant, microorganism, plant cell or plant genome means that the DNA molecule, construct, sequence or protein does not exist in nature as part of such a plant, microorganism, plant cell or plant genome, and/or does not exist in the same physical or genomic location, context or orientation as part of such a plant, microorganism, plant cell or plant genome in nature, without human intervention.

As used herein, the term “chimeric” refers to a single DNA molecule produced by fusing a first DNA molecule to a second DNA molecule, where neither first nor second DNA molecule would normally be found in that configuration fused to the other. The chimeric DNA molecule is thus a new DNA molecule not normally found in nature. An example of a chimeric DNA molecule is a DNA molecule comprising at least one sequence selected from SEQ ID NO:1-10.

As used herein, the term “isolated” in reference to a molecule means that the molecule is at least partially separated from other molecules that are normally associated with it in its native or natural state. In some embodiments, the term “isolated” refers to a DNA molecule that is at least partially separated from the nucleic acids or polynucleotide or DNA sequence(s) that normally flank and are covalently linked to the sequence of the DNA molecule in its native or natural state. An “isolated” DNA molecule may have a DNA sequence corresponding to a portion of the genome of a plant cell without other genomic DNA sequence(s) that normally flank and are covalently linked to the DNA sequence in nature. Such an “isolated” DNA molecule may comprise all or part of a transgene and/or transgenic event, which may comprise all or part of cotton event Gh_CSM63718 or the transgenes or expression cassettes described herein. Nucleic acid sequences or elements, such as a coding sequence, intron sequence, 5′ UTR, promoter sequence, 3′ UTR, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element or subpart is not within the genome of the organism, and at the location within the genome of the organism, in which it is naturally found. An “isolated” DNA molecule may be any recombinant DNA molecule or amplification product or amplicon, and/or may comprise any DNA sequence removed from its natural or biological state and covalently fused to another DNA molecule or sequence with which it is not associated in nature. Such an isolated DNA molecule could be created by the use of biotechnology techniques, such as by making a recombinant DNA or integrating a foreign or heterologous DNA molecule into the chromosome of a cell, plant, or seed. Thus, any DNA molecule comprising a transgenic, recombinant, chimeric or artificial nucleotide sequence, transgene or expression cassette would be considered to be an “isolated” DNA molecule since these sequences are not naturally occurring, regardless of whether the sequence, transgene or expression cassette is present within a plasmid, vector or construct used to transform plant cells, within the genome of a plant, plant part, plant tissue, plant cell or progeny, or is present in detectable amounts in tissues, progeny, biological samples or commodity products derived from a plant, plant part, plant tissue, progeny or plant cell. A recombinant DNA molecule or sequence, or any fragment derived therefrom, comprising all or part of a transgene or junction sequence of the cotton event Gh_CSM63718 would therefore also be considered to be “isolated.” An “isolated” DNA molecule may be extracted or purified from a transgenic plant(s), plant part(s), plant cell(s) and/or tissue(s), or may be present in a homogenate, extract or lysate from any such transgenic plant(s), plant part(s), plant cell(s) and/or tissue(s), or may be produced as an amplicon or amplification product from plant genomic DNA and/or extracted or purified DNA from transgenic plant(s), plant part(s), plant cell(s) and/or tissue(s), or a homogenate, extract or lysate from plant(s), plant part(s), plant cell(s) and/or tissue(s). For the purposes of this disclosure, any transgenic polynucleotide or DNA sequence, i.e., the nucleotide sequence of the DNA inserted into the genome of a plant or bacterium, or present in an extrachromosomal vector, would be considered to be an “isolated” nucleotide or DNA sequence whether it is present within the plasmid or similar structure used to transform the cells, within the genome of the plant or bacterium, or present in detectable amounts in tissues, progeny, biological samples or commodity products derived from the plant or bacterium. An “isolated” DNA molecule is a chemical or biochemical molecule, regardless of whether the molecule is referred to as a nucleic acid, a nucleic acid sequence, a polynucleotide sequence, a DNA sequence, a nucleic acid molecule, a polynucleotide molecule, a DNA molecule, or the like. An “isolated” molecule can provide industrial applicability when present in a plant cell or in a plant genome or when present outside of a plant cell, and therefore, provides and exhibits (and is intended to provide and exhibit) utility regardless of where the molecule is located.

As used herein, the term “correspond” or “corresponding”, or the like, when used in the context of a nucleotide position, mutation, insertion and/or substitution in any given polynucleotide (e.g., SEQ ID NO:9) with respect to a reference polynucleotide sequence (e.g., SEQ ID NO:10) refers to the position(s) of the polynucleotide residue(s) in the given sequence that has identity to the residue(s) in the reference nucleotide sequence when the given polynucleotide is aligned to the reference polynucleotide sequence using a global or local sequence alignment algorithm.

DNA molecules, fragments, and their corresponding DNA sequences, as well as methods of detection are provided. As used herein, the terms “DNA”, “DNA molecule” and “nucleic acid molecule” refer to a deoxyribonucleic acid (DNA) molecule. A DNA molecule may be of genomic or synthetic origin and/or comprise a recombinant or heterologous DNA molecule or sequence. A DNA molecule may be described by convention from the 5′ (upstream) end to the 3′ (downstream) end. As used herein, the term “DNA sequence” refers to the polynucleotide sequence of a DNA molecule, i.e. the sequence of consecutive nucleotides in the DNA molecule. As used herein in reference to nucleotides of a polynucleotide or DNA sequence or molecule, the terms “consecutive” and “contiguous” are interchangeable and synonymous and refer to the 5′ to 3′ order of nucleotides in a polynucleotide or DNA sequence, strand or molecule without any gap or interruption between them. The nomenclature used is that required by Title 37 of the United States Code of Federal Regulations § 1.822 and set forth in the tables in WIPO Standard ST.25 (1998), Appendix 2, Tables 1 and 3. By convention, DNA sequences and fragments thereof are disclosed with reference to the 5′ to 3′ direction of only one strand of the two complementary DNA sequence strands of a DNA molecule. By implication and intent, the complementary sequences of the sequences provided here (the sequences of the complementary strand), also referred to in the art as the reverse complementary or reverse complement sequences, are within the scope of the present disclosure and are expressly intended to be within the scope of the subject matter claimed. As used herein references to SEQ ID NOs:1-10 and fragments thereof include and refer to the sequence of the complementary strand and fragments thereof.

Also provided is a nucleic acid molecule comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of any one of SEQ ID NOs:1-10.

For example, a nucleic acid molecule is provided comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of SEQ ID NO:10 or to the full length of SEQ ID NO: 9.

A DNA molecule, or a fragment derived therefrom, can also be extracted from plant(s), plant part(s), seed(s), progeny or plant cell(s), or a homogenate, extract or lysate from plant(s), plant part(s), plant cell(s) or seed(s) or progeny, or can be produced as an amplicon from extracted, purified or isolated DNA from plant part(s), plant cell(s) and/or tissue(s), progeny, or a homogenate, extract or lysate from plant(s), plant part(s), plant cell(s), progeny and/or seeds, which may further comprise cotton event Gh_CSM63718.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., 1228 S. Park St., Madison, Wis. 53715), and MUSCLE (version 3.6) (Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. Cotton plants, progeny, seeds, cells, plant parts and commodity products comprising a detectable amount of a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9 are within the scope of the presem disclosure.

As used herein, the term “fragment” refers to a smaller piece or sequence of a larger or whole DNA molecule or sequence. For example, a fragment of any one of SEQ ID NOs:1-12 and SEQ ID NOs:14-15 may include a sequence that is at least about 10 consecutive nucleotides, at least about 11 consecutive nucleotides, at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, at least about 100 consecutive nucleotides, at least about 150 consecutive nucleotides, at least about 200 consecutive nucleotides, at least about 250 consecutive nucleotides, at least about 300 consecutive nucleotides, at least about 400 consecutive nucleotides, or at least about 500 consecutive nucleotides of the larger, whole or complete DNA molecule or sequence.

For example, a “fragment” of the transgenic insert sequence (SEQ ID NO: 9) of cotton event Gh_CSM63718 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO: 9. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 9 or any fragment thereof.

Similarly, a fragment of the 5′ flank (SEQ ID NO:11 or SEQ ID NO:14) or 3′ flank (SEQ ID NO:12 or SEQ ID NO:15) of cotton event Gh_CSM63718 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO:11 or SEQ ID NO:14; or SEQ ID NO:12 or SEQ ID NO:15. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO:11 or 12, or SEQ ID NO:14 or 15, or any fragment of either thereof.

As used herein, the term “about” indicates a value or a range of values which would be understood as an equivalent of a stated value and can be greater or lesser than the value or range of values stated. Each value or range of values preceded by the term “about” is also intended to encompass the embodiment of the stated absolute value or range of values.

The term “or” is used herein to mean “and/or” unless explicitly indicated to refer to alternatives only or the alternatives are mutually exclusive. Thus, the term “and/or” as used herein in a phrase such as “X and/or Y” is intended to include “X and Y”, “X or Y”, “X” (alone), and “Y” (alone). Likewise, the term “and/or” as used in a phrase such as “X, Y, and/or Z” is intended to encompass each of the following embodiments: X (alone); Y (alone); Z (alone); X and Y; X and Z; Y and Z; X, Y, and Z; X, Y, or Z; X or Z; Y or Z; Y or Z.

When used in conjunction with the word “comprising” or other open language, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

Cotton event Gh_CSM63718 is characterized as a transgenic insertion into a locus in the cotton genome, resulting in two new junctions (or joining or connection points). The DNA sequence of the region spanning the connection by phosphodiester bond linkage of one end of the transgenic insert to the flanking cotton genomic DNA is referred to herein as a “junction.” In other words, a junction is the connection point or covalent linkage of one end of the transgenic insert and the flanking genomic DNA as one contiguous molecule and is formed by the insertion of a heterologous nucleic acid molecule into the cotton genomic DNA. One junction is found at the 5′ end of the transgenic insert and the other is found at the 3′ end of the transgenic insert, referred to herein as the 5′ and 3′ junctions, respectively. A “junction sequence” refers to a DNA sequence of any length of consecutive nucleotides that spans the 5′ or 3′ junction of a transgenic event in the plant genome. For a “junction sequence” to be specific to a junction between a transgenic event and a flanking genomic sequence, the junction sequence will generally comprise a sufficient number of consecutive nucleotides at one end of the insertion and a sufficient number of consecutive nucleotides of the flanking genomic sequence. According to some embodiments, a “junction sequence” may comprise (i) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides at one end of the insertion and (ii) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides of the flanking genomic DNA sequence, although it is understood that any length of consecutive nucleotides spanning a junction of a transgenic event in a plant genome may be a junction sequence. Junction sequences of cotton event Gh_CSM63718 are not known to appear or exist naturally in the cotton genome or other transgenic cotton events-they are unique to event Gh_CSM63718, and are apparent to, and a variety of junction sequences of cotton event Gh_CSM63718 can be determined by one of skill in the art using SEQ ID NO:10. In SEQ ID NO:10, the 5′ junction is at nucleotides 1,000-1,001, and the 3′ junction is at nucleotides 17,736-17,737. Illustrative junction sequences of cotton event Gh_CSM63718 are provided as SEQ ID NOs:1-8. FIG. 1 illustrates the physical arrangement and locations of the illustrative junction sequences, arranged from 5′ to 3′ (left to right), relative to SEQ ID NO:10. The DNA sequence for the transgenic insert of cotton event Gh_CSM63718 is provided as SEQ ID NO:9. The DNA sequence of the transgenic insert and the cotton genomic DNA flanking each side of the transgenic insert is provided as SEQ ID NO:10. The 5′ junction sequences are provided as SEQ ID NO:1, SEQ ID NO:3, SEQ ID NO:5, and SEQ ID NO:7. The 3′ junction sequences are provided as SEQ ID NO:2, SEQ ID NO:4, SEQ ID NO:6, and SEQ ID NO:8. The junction sequences of cotton event Gh_CSM63718 may be present as part of the genome of a plant, seed, plant part, progeny or plant cell containing cotton event Gh_CSM63718, or a DNA molecule containing all or part of event Gh_CSM63718. The identification of any one or more of the junction sequences in a DNA molecule or a sample from a plant, plant part, seed, progeny, cell or commodity product indicates that the DNA molecule or plant, plant part, seed, progeny, cell or commodity product contains or comprises event Gh_CSM63718, or was obtained from a cotton plant, plant part, seed, progeny, cell or commodity product containing or comprising event Gh_CSM63718, and is diagnostic for the presence of cotton event Gh_CSM63718.

The junction sequences described herein are diagnostic for the presence of all or part of cotton event Gh_CSM63718. Thus, the identification or detection, directly or indirectly, of one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 in a sample or DNA molecule derived from a cotton plant, plant part, seed, progeny, cell, or a commodity product is diagnostic that the cotton plant, plant part, seed, progeny, cell, or a commodity product has or comprises all or part of cotton event Gh_CSM63718. The identification or detection, directly or indirectly, of a 5′ junction sequence and/or a 3′ junction sequence (each as provided or described herein) in a sample or DNA molecule derived from a cotton plant, plant part, seed, progeny, cell, or a commodity product is diagnostic that the cotton plant, plant part, seed, progeny, cell, or a commodity product has or comprises cotton event Gh_CSM63718. The present disclosure thus provides a DNA molecule that comprises at least one of the nucleotide sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Any segment of DNA derived from transgenic cotton event Gh_CSM63718 that is sufficient to include at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 is within the scope of the present disclosure. In addition, any DNA or polynucleotide molecule or sequence comprising a sequence complementary to any of the sequences described herein is also within the scope of the present disclosure.

Polynucleotide molecules are provided, which may be single or double stranded, that can be used either as primers or probes for detecting the presence of DNA comprising all or part of event Gh_CSM63718 in a sample derived from a cotton plant, plant part, seed, progeny, cell, or a commodity product. Such primers or probes are specific for a target polynucleotide sequence and, as such, are useful for the identification of cotton event Gh_CSM63718 polynucleotide by the methods described herein. A primer or probe can hybridize to a target polynucleotide sequence to allow for specific detection or amplification of a polynucleotide molecule that comprises, or is covalently linked and associated with, the target polynucleotide sequence. The primers and/or probes may be chosen to identify and distinguish detection of a particular transgenic event and not only the presence of a transgene in a plant genome. The target polynucleotide sequence may comprise all or part of cotton event Gh_CSM63718, a junction sequence and/or flanking genomic DNA. Probes and primers according to the present disclosure may have (i) complete or 100% sequence complementarity (i.e., 100% complementary) to a target polynucleotide sequence or (ii) incomplete sequence complementarity to a target polynucleotide, such as at least 60% complementary, at least 65% complementary, at least 70% complementary, at least 75% complementary, at least 80% complementary, at least 85% complementary, at least 90% complementary, at least 95% complementary, or at least 99% complementary to the target polynucleotide sequence as long as the probe or primer has sufficient complementarity to the target polynucleotide sequence to hybridize to the target polynucleotide sequence under stringent hybridization conditions that are suitable and necessary for use of the probe or primer in the relevant amplification or detection assay, reaction or method. As understood in the art, the percentage complementarity of a primer or probe may be lower if the length of the primer or probe is longer and depends on the stringency and use. Provided are illustrative polynucleotide molecules that can be used either as primers or probes for detecting the presence of cotton event Gh_CSM63718 in a sample. Detection of the presence of cotton event Gh_CSM63718 may be done by using methods known in the art, such as thermal or isothermal amplification of nucleic acids or nucleic acid hybridization techniques (such as Northern analysis and Southern analysis).

A “probe” is a nucleic acid molecule that is complementary to a strand of a target nucleic acid and is useful in hybridization detection methods. Probes include not only deoxyribonucleic or ribonucleic acids but also polyamides and other probe materials that bind specifically to a target DNA sequence and the detection of such binding can be useful in detecting the presence or absence of the target DNA sequence. A probe may be attached to a conventional detectable label or reporter molecule, such as a radioactive isotope, ligand, chemiluminescent agent, or enzyme. Such a probe is complementary to a strand of a target nucleic acid and, in the case of the present disclosure, to a strand of DNA from event Gh_CSM63718 whether from an event Gh_CSM63718-containing plant or from a sample that includes event Gh_CSM63718 DNA.

Provided herein is a DNA molecule comprising a polynucleotide segment of sufficient length to function as a DNA probe that hybridizes specifically under stringent hybridization conditions with cotton event Gh_CSM63718 DNA in a sample, wherein detecting hybridization of the DNA molecule under the stringent hybridization conditions is diagnostic for the presence of cotton event Gh_CSM63718 in the sample. Also provided is a DNA molecule comprising a polynucleotide segment of sufficient length to function as a DNA probe specific for detecting in a sample at least one of: (i) a 5′ junction sequence between flanking cotton genomic DNA and the transgenic insert of cotton event Gh_CSM63718; (ii) a 3′ junction sequence between the transgenic insert of cotton event Gh_CSM63718 and flanking cotton genomic DNA; (iii) SEQ ID NO:9; and (iv) a fragment of SEQ ID NO:9 comprising a sufficient length of contiguous nucleotides of SEQ ID NO:9 to identify the sequence as a fragment of the transgenic insert of Gh_CSM63718. An illustrative DNA sequence useful as a probe for detecting cotton event Gh_CSM63718 is provided as SEQ ID NO:21. Other DNA sequences useful as probes for detecting cotton event Gh_CSM63718 include SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and complements of any of the foregoing.

A “primer” is a DNA molecule or oligonucleotide that is designed for use in specific annealing or hybridization methods that involve an in vitro amplification reaction. A pair of primers may be used with template DNA (such as a sample of cotton event Gh_CSM63718 genomic DNA) in a thermal amplification reaction (such as polymerase chain reaction (PCR)) or any other suitable amplification method known in the art to produce an amplification product or amplicon, where the amplicon produced from such reaction would have a DNA sequence corresponding to sequence of the template DNA located between the two sites where the primers hybridized to the template DNA.

DNA amplification reactions, methods and techniques are known to those skilled in art. DNA amplification can be accomplished by any of the various nucleic acid amplification methods known in the art, including thermal and isothermal amplification methods such as polymerase chain reaction (PCR) and loop-mediated isothermal amplification (LAMP). Amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb (kilobase) of genomic DNA and up to 42 kb of bacteriophage DNA (Cheng et al., 1994). These methods as well as other methods known in the art of DNA amplification may be used in the practice of the present disclosure. Examples of DNA amplification methods include PCR, Recombinase Polymerase Amplification (RPA) (see for example U.S. Pat. No. 7,485,428), Strand Displacement Amplification (SDA) (see for example, U.S. Pat. Nos. 5,455,166 and 5,470,723), Transcription-Mediated Amplification (TMA) (see for example, Guatelli et al., 1990), Rolling Circle Amplification (RCA) (see for example, Fire and Xu, 1995; Liu, et al., 1996; Lizardi, et al., 1998; U.S. Pat. Nos. 5,714,320 and 6,235,502), Helicase Dependent Amplification (HDA) (see for example Vincent et al., 2004; U.S. Pat. No. 7,282,328), Multiple Displacement Amplification (MDA) (see for example Dean et al., 2002) and Loop-Mediated Isothermal Amplification (LAMP) (see for example Notomi et al., 2000). A sequence of the heterologous DNA insert and/or flanking genomic DNA sequence from cotton event Gh_CSM63718 can be verified or tested by amplifying such DNA molecules from cotton seed containing event Gh_CSM63718 DNA or cotton plants grown from the cotton seed containing event Gh_CSM63718 DNA, using primers derived from the sequences provided herein, followed by standard DNA sequencing of the PCR amplicon or a cloned DNA fragment thereof.

As used herein, an “amplification product” or “amplified DNA” or “amplicon” refers to the nucleic acid or DNA molecule or segment produced by a nucleic acid amplification reaction or method as further described herein, which is directed to a target nucleic acid or DNA molecule that is part of a template nucleic acid molecule. Amplification or amplifying refers to making multiple copies of a target DNA molecule or segment from a template DNA. For example, to determine whether a cotton plant, plant part, seed, progeny or plant cell, resulting from selfing or outcross of a parent comprising cotton event Gh_CSM63718 contains cotton event Gh_CSM63718, DNA may be extracted from the cotton plant tissue sample and subjected to an amplification reaction or method using a pair of primers that are specific for a target sequence that is uniquely associated or part of cotton event Gh_CSM63718, such as, for example, a first primer derived from a genomic DNA sequence in the region flanking the heterologous inserted DNA of cotton event Gh_CSM63718 that is elongated by polymerase 5′ to 3′ in the direction of the inserted DNA, and a second primer derived from the heterologous inserted DNA molecule that is elongated by the polymerase 5′ to 3′ in the direction of the flanking genomic DNA from which the first primer is derived. The amplicon may range in length depending on the length of the intervening polynucleotide or DNA sequence between the two primer target sequences in the template DNA molecule. Alternatively, a primer pair can be derived from the genomic sequence on both sides of the inserted heterologous DNA so as to produce an amplicon that includes the entire insert polynucleotide sequence (e.g., a forward primer targeted to the genomic portion on the 5′ end of SEQ ID NO:10 (i.e. upstream of SEQ ID NO:9) and a reverse primer targeted to the genomic portion on the 3′ end of SEQ ID NO:10 (i.e. downstream of SEQ ID NO:9) that amplifies a DNA molecule comprising the inserted DNA sequence (SEQ ID NO:9) identified herein in the cotton event Gh_CSM63718 genome. The use of the term “amplicon” specifically excludes primer dimers that may be formed in a DNA amplification reaction.

Provided herein is a pair of DNA molecules comprising a first DNA molecule and a second DNA molecule, wherein the first and the second DNA molecules are different from one another and comprise a fragment of SEQ ID NO:10 or a complement thereof and function as DNA primers when used together in an amplification reaction with DNA comprising cotton event Gh_CSM63718 to produce an amplicon diagnostic for cotton event Gh_CSM63718 in a sample. For example, the first and second DNA molecules can comprise SEQ ID NO:19 and SEQ ID NO:20. The amplicon described herein may comprise a DNA sequence comprising one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 wherein the fragment is at least 10 nucleotides in length and comprises nucleotides 1,000-1,001 or 17,736-17,737 of SEQ ID NO:10. According to present embodiments, the sequence of an amplicon comprises at least one junction sequence or two junction sequences, such as a 5′ junction sequence and/or a 3′ junction sequences for cotton event Gh_CSM63718. Amplification and detection of such an amplicon is indictive or diagnostic for cotton event Gh_CSM63718.

For practical purposes, one should design primers which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TaqMan®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. Any primer pair of forward and reverse primers, which may be identical or complementary to part of SEQ ID NO:10, such as SEQ ID NO:19 and SEQ ID NO:20, that is useful in a DNA amplification method to produce an amplicon diagnostic for cotton event Gh_CSM63718 or progeny thereof is an aspect of the disclosure. Any single isolated DNA polynucleotide primer molecule comprising at least 15 contiguous nucleotides of SEQ ID NO:10, or its complement that is useful in a DNA amplification method to produce an amplicon diagnostic for cotton event Gh_CSM63718 or progeny thereof is an aspect of the disclosure. Any single isolated DNA polynucleotide primer molecule comprising at least 15 contiguous nucleotides of SEQ ID NO:11 or SEQ ID NO:12, or its complement that is useful in a DNA amplification method to produce an amplicon diagnostic for plants comprising cotton event Gh_CSM63718 or progeny thereof is an aspect of the disclosure. Any single isolated DNA polynucleotide primer molecule comprising at least 15 contiguous nucleotides of SEQ ID NO:9, or its complement that is useful in a DNA amplification method to produce an amplicon diagnostic for cotton event Gh_CSM63718 or progeny thereof is an aspect of the disclosure.

A primer is typically designed to hybridize specifically to a complementary target DNA strand to form a hybrid between the primer and the target DNA strand. Hybridization or binding of a primer to the complementary target DNA strand is a point of recognition by a polymerase to begin extension of the primer (i.e., polymerization of additional nucleotides into a lengthening nucleotide molecule) using the target DNA strand as a template. Primer pairs refer to use of two primers binding opposite strands of a double stranded nucleotide segment for the purpose of amplifying the polynucleotide segment between the positions targeted for binding by the individual members of the primer pair, typically in a thermal amplification reaction or other conventional nucleic-acid amplification methods. Primer pairs are typically designed to hybridize to different nearby target positions of a template DNA molecule on opposing strands of the template DNA molecule such that the intervening region or sequence between the two primers can be specifically amplified for use or detection through multiple rounds of amplification.

To detect the presence or absence of cotton event Gh_CSM63718, the target positions and/or the intervening region or sequence of a template DNA molecule may comprise at least one junction sequence and/or at least a portion of the insert of cotton event Gh_CSM63718. To detect the absence of cotton event Gh_CSM63718, the target positions and/or the intervening region or sequence of a template DNA molecule may comprise cotton genomic DNA that does not include a junction sequence or any portion of the insert of cotton event Gh_CSM63718. Thus, the presence or absence of an amplicon with a primer pair may be diagnostic of the presence or absence, respectively, of cotton event Gh_CSM63718 in a DNA molecule or sample, or vice versa. This may also be possible with more than one primer pair. For example, a first primer pair may produce a first amplicon if cotton event Gh_CSM63718 is present, and a second primer pair may produce a second amplicon if cotton event Gh_CSM63718 is absent or not present. Alternatively, the size of an amplicon produced in an amplification reaction may also be diagnostic of the presence or absence of cotton event Gh_CSM63718 in a DNA molecule or sample—e.g., a primer pair may produce a first amplicon of a first size if cotton event Gh_CSM63718 is present or a second amplicon of a second size if cotton event Gh_CSM63718 is absent and not present; or a first primer pair may produce a first amplicon of a first size if cotton event Gh_CSM63718 is present, and a second primer pair may produce a second amplicon of a second size if cotton event Gh_CSM63718 is absent or not present. According to some of these embodiments, at least two primer pairs may be used wherein at least one of the primer pairs is used as an internal control and is not associated with cotton event Gh_CSM63718.

According to present embodiments, a primer pair to detect the presence or absence of all or part of cotton event Gh_CSM63718 in a DNA molecule or sample comprises a first primer and a second primer, wherein the first primer is complementary to a 5′ flanking genomic DNA sequence and the second primer is complementary to a sequence within the transgenic insert; or wherein the first primer is complementary to a 5′ flanking genomic DNA sequence and the second primer is complementary to a 3′ flanking genomic DNA sequence; or wherein the first primer is complementary to a sequence within the transgenic insert and the second primer is complementary to a 3′ flanking genomic DNA sequence. Each reference in this paragraph to a primer complementary to a 5′ flanking genomic DNA sequence, a 3′ flanking genomic DNA sequence, or a sequence within the transgenic insert of cotton event Gh_CSM63718 is also intended to potentially include a primer complementary to the reverse complement or opposing strand of the respective 5′ flanking genomic DNA sequence, 3′ flanking genomic DNA sequence, or sequence within the transgenic insert of cotton event Gh_CSM63718.

Illustrative DNA molecules useful as primers for detection of cotton event Gh_CSM63718 are provided as SEQ ID NO:19 and SEQ ID NO:20. The primer pair SEQ ID NO:19 and SEQ ID NO:20 can be useful as a first primer (complementary to a sequence within the 3′ flanking genomic DNA sequence) and a second primer (corresponding to a sequence within the transgenic insert), wherein each primer has sufficient length of consecutive nucleotides of SEQ ID NO:10 or a sequence complementary to SEQ ID NO:10 to function as DNA primers that, when used together in an amplification reaction with template DNA derived from cotton event Gh_CSM63718, hybridize to opposite strands of the template DNA and produce an amplicon diagnostic for cotton event Gh_CSM63718 DNA in a sample. The primer pair SEQ ID NO:19 (complementary to a 3′ flanking genomic DNA sequence) and SEQ ID NO:22 (corresponding to a 5′ flanking genomic DNA sequence) are useful as a first primer and a second primer, wherein each primer has sufficient length of consecutive nucleotides of a locus within the cotton genome to function as DNA primers that, when used together in a thermal amplification reaction with template DNA, to produce an amplicon indictive or diagnostic for the wildtype DNA for the zygosity of Gh_CSM63718 event DNA in a sample. An amplicon diagnostic for event Gh_CSM63718 comprises a sequence not naturally found in the cotton genome.

A primer may further comprise an oligo tail sequence such as those used in the Kompetitive Allele-Specific PCR (KASP™) method. The allele-specific primers each harbor a unique tail sequence that corresponds with a universal FRET (fluorescence resonant energy transfer) cassette; one labelled with FAM™ dye and the other with HEX™ dye. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is no longer quenched and emits fluorescence.

Methods for designing and using primers and probes are well known in the art. DNA molecules comprising fragments of SEQ ID NOs:1-10 are useful as primers and probes for detecting cotton event Gh_CSM63718 and can readily be designed by one of skill in the art using the sequences provided herein. Such probes and primers are selected to be of sufficient length and sequence complementarity to a target sequence to hybridize specifically to a target sequence under stringency hybridization conditions. Probes and primers may have a complete sequence complementarity or identity with the target sequence, although probes and primers differing from the target sequence in terms of identity or complementarity but retaining the ability to form a stable double-stranded structure under particular hybridization conditions or reaction conditions and to hybridize to the target sequence may be designed by conventional methods.

Any conventional nucleic acid hybridization or amplification method can be used to identify or detect the presence of a target DNA from a transgenic plant, such as cotton event Gh_CSM63718, in a sample. Polynucleotide molecules or DNA molecules, also referred to as “polynucleotide segment or fragment of sufficient length” or “sufficient length of contiguous or consecutive nucleotides” therefore are capable of specifically hybridizing to a target DNA sequence under certain hybridization conditions or reaction conditions. As used herein, the term “of sufficient length” refers to any length that is sufficient to be useful in a detection method of choice. Probes and primers are generally at least about 8 nucleotides, at least about 10 nucleotides, at least about 12 nucleotides, at least about 14 nucleotides, at least about 16 nucleotides, at least about 18 nucleotides, at least about 20 nucleotides, at least about 22 nucleotides, at least about 24 nucleotides, at least about 26 nucleotides, at least about 28 nucleotides, or at least about 30 nucleotides or more in length. Such probes and primers hybridize specifically to a target DNA sequence under stringent hybridization conditions.

As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two nucleic acid molecules exhibit “complete complementarity” and are “completely complementary” if every nucleotide of the first nucleic acid molecule is complementary to every nucleotide of the second nucleic acid molecule when they are aligned. Two molecules are “minimally complementary” if they can hybridize to one another with sufficient stability to permit them to remain annealed to one another under at least conventional “low stringency” conditions. Similarly, the molecules are “complementary” if they can hybridize to one another with sufficient stability to permit them to remain annealed to one another under conventional “high stringency” conditions. Conventional stringency conditions are described by Haymes et al., In: Nucleic Acid Hybridization, A Practical Approach, IRL Press, Washington, DC (1985), and by MR Green and J Sambrook, Molecular cloning: a laboratory manual, 4^{th} Edition, Cold Spring Harbor Laboratory Press, Cold Spring Harbor, N.Y. (2012). Departures from complete complementarity are therefore permissible, as long as such departures do not completely preclude the capacity of the molecules to form a double-stranded structure. In order for a nucleic acid molecule to serve as a primer or probe, it need only be sufficiently complementary in sequence to be able to form a stable double-stranded structure under the particular solvent and salt concentrations and other conditions employed.

As used herein, a substantially homologous or complementary sequence in relation to a reference nucleic acid sequence is a nucleic acid sequence that will specifically hybridize to the reference nucleic acid sequence or its complement to which it is being compared under high stringency conditions. As used herein, “stringent hybridization conditions” refers to conditions under which a polynucleotide will hybridize to its target sequence, typically in a complex mixture of nucleic acids, but to essentially no other sequences. “Stringent conditions” or “stringent hybridization conditions” when referring to a polynucleotide probe, refer to conditions under which a probe will hybridize to its target sequence to a detectably greater degree than to other sequences (e.g., at least 2-fold over background). Stringent conditions are sequence-dependent and will be different in different circumstances. Longer sequences hybridize specifically at higher temperatures, Generally, stringent conditions are selected to be about 5-10° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8,3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing).

Appropriate stringency conditions which promote DNA hybridization, for example, 6×sodium chloride/sodium citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Regarding the amplification of a target polynucleotide (e.g., by PCR) using a particular amplification primer pair, “stringent conditions” or “stringent hybridization conditions” are conditions that permit the primer pair to hybridize to the target polynucleotide to which a primer having the corresponding sequence (or its complement) would bind and to produce an identifiable amplification product (the amplicon) having a cotton Gh_CSM63718 event specific region in a DNA thermal amplification reaction. The term “specific for” a target sequence indicates that a probe or primer hybridizes under stringent hybridization conditions only to the target sequence in a sample comprising the target sequence.

A polynucleotide molecule or DNA molecule of the present disclosure, such as a primer or a probe, will specifically hybridize to at least one of the nucleic acid molecule sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO:10, or a complete complement of or fragment of any of the foregoing under stringent hybridization conditions, or under moderately stringent hybridization conditions if the sequence of the polynucleotide molecule is not identical to the at least one of the nucleic acid molecules. The hybridization of a nucleic acid molecule, such as a primer or probe, to the target DNA molecule can be detected by any number of methods known to those skilled in the art, which can include, but are not limited to, fluorescent tags, radioactive tags, antibody-based tags, and chemiluminescent tags.

An illustrative DNA molecule or polynucleotide useful as a probe for detecting cotton event Gh_CSM63718 is provided as SEQ ID NO:21. In some embodiments, a DNA molecule that functions as a probe comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complement of any of the foregoing or a fragment of any of the foregoing. In other embodiments, a DNA molecule comprises a polynucleotide segment of sufficient length to function as a DNA probe specific for at least one of: a) a 5′ junction sequence between flanking cotton genomic DNA and the transgenic insert of cotton event Gh_CSM63718; b) a 3′ junction sequence between the transgenic insert of cotton event Gh_CSM63718 and flanking cotton genomic DNA; c) SEQ ID NO:9; or d) a fragment of SEQ ID NO:9 comprising a sufficient length of contiguous nucleotides of SEQ ID NO:9 to identify the sequence as a fragment of the transgenic insert of Gh_CSM63718 in a sample of DNA.

A diagnostic amplicon produced by the methods described herein may be detected by a plurality of techniques known in the art, such as sequencing, restriction mapping, Southern analysis, or any other suitable polynucleotide or DNA hybridization, blotting, polymerization and/or amplification-based approach or technique. One method is Genetic Bit Analysis (Nikiforov et al., 1994) where a DNA oligonucleotide is designed that overlaps both the adjacent flanking genomic DNA sequence and the inserted DNA sequence—i.e., a junction sequence. The oligonucleotide is immobilized in wells of a microtiter plate. Following PCR of the region of interest (using, for example, one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic junction sequence due to successful amplification, hybridization, and single base extension.

Another method is the pyrosequencing technique as described by Winge (2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen et al. (1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time polymerase chain reaction (PCR) has the ability to monitor the progress of the PCR as it occurs (i.e., in real time). Data are collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is a method of detecting and quantifying the presence of a DNA sequence using real-time PCR and is fully described in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermal stable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular beacons have been described for use in sequence detection as described in Tyangi et al. (1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results and indicates the presence of the flanking/transgene insert sequence due to successful amplification and hybridization.

Other detection methods known in the art may be used. For example, microfluidics (see, e.g., U.S. Patent Publication No. 2006/068398; U.S. Pat. No. 6,544,734) provide methods and devices that can be used to separate and amplify DNA samples or molecules. Optical dyes can be used to detect and measure specific DNA molecules (see, e.g., WO/05017181). Nanotube devices (see, e.g., WO/06024023) that comprise an electronic sensor for the detection of DNA molecules or nanobeads that bind specific DNA molecules can then be detected. Nanopore sequencing technology, such as that described in Wang et al. (2021), Tayler et al. (2018), or Pearson et al. (2019), can also be used for event detection.

The DNA molecules and corresponding nucleotide sequences provided herein are therefore useful for, among other things, identifying cotton event Gh_CSM63718, detecting the presence of DNA derived from the transgenic cotton event Gh_CSM63718 in a sample, and monitoring samples for the presence and/or absence of cotton event Gh_CSM63718 or plant parts derived from cotton plants comprising event Gh_CSM63718.

Provided are proteins that can be used to produce antibodies for detecting the presence of cotton event Gh_CSM63718 in a sample. Such antibodies are specific for the PAT, DMO, TDO, EPSPS or PPO proteins that are encoded by cotton event Gh_CSM63718. Methods for preparing a polyclonal antibody or a monoclonal antibody are well known to those skilled in the art and can be used to make antibodies specific for the PAT, DMO, TDO, EPSPS or PPO proteins encoded by cotton event Gh_CSM63718. For example, U.S. Pat. No. 9,371,394 describes antibodies to the PAT enzyme; U.S. Pat. No. 7,838,729 and Wang et al. (2016) describe antibodies to DMO; USDA APHIS Petition 04_08601p describes antibody to CP4 EPSPS; Lermontova et al (1997) describes antibodies to a PPO protein. The DNA sequence encoding the PAT, DMO, TDO, EPSPS and PPO proteins are provided in SEQ ID NO:10 and the start positions and stop positions of the coding sequences are indicated in Table 1. The DNA sequences encoding the proteins and the proteins encoded by the sequences are useful to produce antibodies for detecting the presence of cotton event Gh_CSM63718 by the methods described herein. Detection for the presence of cotton event Gh_CSM63718 may be done by using any protein detection techniques known in the art, such as Western blot analysis, immuno-precipitation, enzyme-linked immunosorbent assay (ELISA), antibody attachment to a detectable label or reporter molecule (such as a radioactive isotope, ligand, chemiluminescent agent, or enzyme), or enzymatic action on a reporter molecule. One method provides for contacting a sample with an antibody or antibodies that bind to at least one of the PAT, DMO, TDO, EPSPS and PPO proteins encoded by cotton event Gh_CSM63718 and then detecting the presence or absence of antibody binding. The binding of such antibody is diagnostic for the presence of one or more proteins encoded by cotton event Gh_CSM63718.

An alternative to antibody for protein detection is the aptamer-based detection method for detecting proteins or molecules of interest in a sample. As used herein, the term “aptamer(s)” or “aptamer sequences(s)” refers to short synthetic single-stranded oligonucleotide molecules with high-affinity and specificity binding to a target molecule such as a protein, polypeptide, lipid, glycoprotein, glycolipid, glycopeptide, saccharide, or polysaccharide by forming distinct tertiary structures (Ellington and Szostak, 1990; Robertson and Joyce, 1990; Tuerk and Gold, 1990; Wang et al., 2019). The single-stranded nucleic acid can be ssDNA, RNA or derivatives of either thereof. The aptamer comprises a three-dimensional structure held in certain conformation(s) that provide intermolecular contacts to specifically bind its given target. Although aptamers are nucleic acid-based molecules, the binding to the target molecule is not entirely dependent on a linear base sequence, but rather a particular secondary/tertiary/quaternary structure. The term aptamer also covers next generation aptamers such as X aptamers that cannot typically be amplified by PCR but can be adapted by adding a link primer. Such aptamers can specifically bind to proteins of interest but can also be easily amplified, sequenced etc. in a downstream process. The term aptamer also covers aptamers that include modified bases. It is envisaged that the aptamers may include traditional aptamers of 15 to 120 bases in length, as well as longer aptamers of approximately 200 bases in length (e.g., Ultramers® by Integrated DNA Technologies, Inc. Coralville, Iowa, USA). To detect the protein of interest in a sample, aptamers specific to the protein of interest are obtained and are then incubated with the sample. If the protein of interest is present in the sample, protein-aptamer conjugates are formed. Methods for detecting the aptamer/protein complex are known in the art, such as aptablotting or South-Western blot (Li et al., 2017; Sekhon et al., 2017), aptamer-based Western blot (Wang at al., 2020), aptamer sandwich assay (Svobodova et al., 2021). Chemical modifications, additional functional groups, and/or linkers can be added to the nucleic acid aptamer to provide increased binding affinity to a target protein, and to provide a convenient means to detect the target molecule. Such tags and or labels can comprise fluorescent, luminescent, absorbance, or radioactive-based chemical groups, or can comprise an enzyme or substrate that provides a detectable response such as a precipitate, either alone or in the presence of other factors.

Nucleic acid or protein detection kits for detecting the presence of cotton event Gh_CSM63718 are provided. Variations on such kits can also be developed using the compositions and methods disclosed herein and the methods well known in the art for protein and nucleic acid detection for identification of cotton event Gh_CSM63718. Protein and nucleic acid detection kits can be applied to methods for breeding with plants comprising cotton event Gh_CSM63718. Such kits contain primers and/or probes or antibodies or aptamers which are specific to cotton event Gh_CSM63718. Such DNA primers and/or probes may comprise fragments of one or more of SEQ ID NOs:1-10, or antibodies specific for a protein encoded by the cotton event Gh_CSM63718. The kits can also contain instructions for using the primers, probes, antibodies, or aptamers for detecting the presence of cotton event Gh_CSM63718. Kits may optionally also comprise reagents for performing the detection or diagnostic reactions described herein.

One example of a detection kit comprises at least one DNA molecule of sufficient length of contiguous nucleotides of SEQ ID NO:10 to function as a DNA probe useful for detecting the presence or absence of cotton event Gh_CSM63718 in a sample. The DNA derived from transgenic cotton plants comprising event Gh_CSM63718 would comprise a DNA molecule having at least one sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, a complement of any of the foregoing, or a fragment of any of the foregoing. An illustrative DNA molecule sufficient for use as a probe is one comprising the sequence provided as SEQ ID NO:21. Other probes may be readily designed by one of skill in the art. The probe can include a junction sequence that spans the 5′ or 3′ junction between the cotton genomic DNA and the transgenic insert of cotton event Gh_CSM63718.

Another example of a detection kit comprises at least one primer pair that specifically hybridize to a target DNA and amplify a diagnostic amplicon under the appropriate reaction conditions useful for detecting the presence or absence of cotton event Gh_CSM63718 in a sample. A kit that contains DNA primers that are homologous or complementary to any portion of the cotton genomic region as set forth in SEQ ID NO:11 or 12 and to any portion of the inserted transgenic DNA as set forth in SEQ ID NO:9 is an object of the present disclosure. The kit may provide an agarose gel-based detection method or any number of methods of detecting the amplicon that are known in the art. Such a method may also include sequencing the amplicon or a fragment thereof. Illustrative DNA molecules sufficient for use as a primer pair are ones comprising the sequences provided as SEQ ID NO:19 and SEQ ID NO:20, and SEQ ID NO:19 and SEQ ID NO:22, respectively, wherein the primer pair SEQ ID NO:19 and SEQ ID NO:20 will produce an amplicon diagnostic for the presence of event Gh_CSM63718 in a sample; and the primer pair SEQ ID NO:19 and SEQ ID NO:22 will produce an amplicon indictive of wild-type DNA, therefore, diagnostic for the absence of event Gh_CSM63718 in a sample. Other primer pairs may be readily designed by one of skill in the art.

Another example of a detection kit comprises at least one antibody specific for the PAT, TDO, DMO, EPSPS and/or PPO proteins encoded by cotton event Gh_CSM63718. For example, such a kit may utilize a lateral flow strip comprising reagents activated when the tip of the strip is contacted with an aqueous solution. Illustrative proteins for use in antibody production are the PAT, TDO, DMO, EPSPS, and PPO proteins encoded by the sequence provided as SEQ ID NO:10, or any fragment thereof. For example, an antibody specific for the TDO protein encoded by cotton event Gh_CSM63718 and/or an antibody specific for the PPO protein encoded by cotton event Gh_CSM63718 can be used. Detection of binding of the antibody to the one or more proteins encoded by cotton event Gh_CSM63718 in a sample is diagnostic for the presence of cotton event Gh_CSM63718 in the sample.

The detection kits provided herein are useful for, among other things, identifying cotton event Gh_CSM63718, selecting plant varieties or hybrids comprising cotton event Gh_CSM63718, detecting the presence of DNA derived from the transgenic cotton plant comprising event Gh_CSM63718 in a sample, and monitoring samples for the presence and/or absence of cotton plants comprising event Gh_CSM63718, or plant parts derived from cotton plants comprising event Gh_CSM63718.

Cotton plants, progeny, seeds, cells, and plant parts comprising cotton event Gh_CSM63718 are provided, as well as commodity products produced using these. As used herein, the term “cotton” or “cotton” means plant species within Gossypium hirsutum, Gossypium barbadense, Gossypium arboretum, Gossypium herbaceum and all plant varieties belonging to the genus Gossypium that can be bred with Gossypium hirsutum plants. The term “cotton” is intended to include cotton plants, plant parts, plant cells, plant tissue, seeds, progeny plants, and/or cotton commodity products. These cotton plants, plant parts, plant cells, plant tissues, seeds, progeny plants and commodity products contain or comprise cotton event Gh_CSM63718 or are derived from a transgenic cotton plant, plant part, plant cell, plant tissue, seed, progeny plant or commodity product containing or comprising event Gh_CSM63718. These cotton plants, plant parts, plant cells, plant tissues, seeds, progeny plants and commodity products contain a detectable amount of a polynucleotide or DNA molecule comprising at least one junction sequence and/or heterologous transgenic insert sequence of cotton event Gh_CSM63718, such as a polynucleotide or nucleic acid or DNA molecule having or comprising at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 16 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, a polynucleotide comprising a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing. In some embodiments, the cotton plant, plant part, plant cell, plant tissue, or seed is further defined as a progeny plant of any generation of a cotton plant comprising cotton event Gh_CSM63718, or a cotton plant part, plant seed, or plant cell derived therefrom.

As used herein, “cotton event Gh_CSM63718” or “cotton event Gh_CSM63718 locus” refers to the genomic locus of the cotton event Gh_CSM63718 or a modified cotton event Gh_CSM63718 comprising the complete or partial flanking, junction and insertion sequences of the cotton event Gh_CSM63718 or the modified cotton event Gh_CSM63718. A modified cotton event Gh_CSM63718 comprises one or more mutations, edits and/or genetic modifications such as deletion, insertion, inversion or transposition in the cotton event Gh_CSM63718 locus relative to the cotton event Gh_CSM63718.

A modified cotton event Gh_CSM63718 and methods of making a modified cotton event Gh_CSM63718 are provided. As is described further herein, various mutagenesis or targeted genome editing techniques and related tools exist or could be made or engineered to permit genetic modification or mutation of the transgenic insert, junction and/or the flanking genomic DNA of cotton event Gh_CSM63718, such as by deletion, insertion, transposition, inversion, and/or substitution of nucleic acid sequence(s), or by insertion or introduction of a guide RNA target site or a cognate guide RNA recognition target (CgRRS), and the transgenic event as modified may still be uniquely characterized by the presence of heterologous DNA and/or one or more sequences of the insertion, junction(s) and/or flanking sequence described herein at the particular position in the genome previously occupied by the unmodified cotton event Gh_CSM63718 relative to flanking portions or sequences of the native cotton genome. According to present embodiments, a modified transgenic event derived from cotton event Gh_CSM63718 may comprise all or part of the insertion sequence and/or transgene cassette of cotton event Gh_CSM63718 and/or one or more complete or partial flanking sequences described herein. As used herein, a “modified cotton event Gh_CSM63718” refers to the genomic DNA or sequence of the cotton event Gh_CSM63718 locus comprising one or more mutations, edits, or genetic modifications relative to the genomic DNA or sequence of the cotton event Gh_CSM63718, wherein such mutations or edits or genetic modifications are introduced or made by a mutagenesis or targeted genome editing technique of a cotton plant, plant part, tissue or cell comprising the cotton event Gh_CSM63718. A “modified cotton event Gh_CSM63718” includes a “further modified cotton event Gh_CSM63718” made by first inserting a target site or cognate target site or CgRRS into the cotton event Gh_CSM63718 locus and then further modifying the cotton event Gh_CSM63718 locus as described herein. Methods and techniques of mutagenesis are known in the art and include, for example, chemical mutagenesis (i.e., treatment with a chemical mutagen, such as an azide, hydroxylamine, nitrous acid, acridine, nucleotide base analog, or alkylating agent—e.g., EMS (ethylmethane sulfonate), MNU (N-methyl-N-nitrosourea), etc.), physical mutagenesis (e.g., gamma rays, X-rays, UV, ion beam, other forms of radiation, etc.), insertional mutagenesis (e.g., transposon or T-DNA insertion), and site directed mutagenesis through genome editing. Cotton plants, plant parts, plant seeds, plant tissues, and plant cells described herein include cotton plants, parts, seeds, tissues and cells comprising a modified cotton event Gh_CSM63718 or a further modified cotton event Gh_CSM63718.

The cotton plants, plant parts, plant cells, plant tissues, seeds, progeny plants and commodity products express or comprise at least one herbicide tolerance gene selected from the group consisting of PAT, TDO, DMO, EPSPS, PPO, and any combination thereof, and are tolerant to at least one herbicide selected from the group consisting of glufosinate, a ß-triketone HPPD inhibitor such as mesotrione, dicamba, glyphosate, a PPO herbicide, and any combination thereof. Illustrative PPO herbicides include, for example, but are not limited to flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, trifludimoxazin, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate, and combinations of any thereof.

The present disclosure provides cotton plants, progeny, seeds, plant cells, and plant parts such as microspores, pollen, anthers, ovules, ovaries, flowers, bolls, embryos, stems, buds, nodes, leaves, roots, and calli derived from a transgenic cotton plant comprising cotton event Gh_CSM63718. A representative sample of seed comprising cotton event Gh_CSM63718 has been deposited according to the Budapest Treaty for the purpose of enabling the present disclosure. The ATCC repository has assigned Accession No. PTA-127638 to the seed comprising cotton event Gh_CSM63718.

A microorganism is provided. The microorganism comprises a polynucleotide molecule having the nucleotide sequence of SEQ ID NO:9, or a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:9. An example of such a microorganism is an Agrobacterium cell. Another example of such a microorganism is an E. coli cell.

A plant cell is provided comprising a polynucleotide molecule as described herein. For example, a plant cell is provided having a nucleotide sequence present in its genome, wherein the nucleotide sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a nucleic acid molecule comprising a polynucleotide having a nucleotide sequence that is at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% identical to the full length of SEQ ID NO:10 or the full length of SEQ ID NO: 9.

Plant cells and microorganisms of the present disclosure are useful in many industrial applications, including but not limited to: (i) use as research tools for scientific inquiry or industrial research; (ii) use in culture for producing endogenous or recombinant carbohydrate, lipid, nucleic acid, enzymes or protein products or small molecules that may be used for subsequent scientific research or as industrial products; and (iii) for the plant cells of the present disclosure, use with modern plant tissue culture techniques to produce transgenic plants or plant tissue cultures that may then be used for agricultural research or production. The production and use of such transgenic plant cells utilize modern microbiological techniques and human intervention to produce a man-made, unique plant cell. In this process, a recombinant DNA is inserted into a plant cell's genome to create a transgenic plant cell that is separate and unique from naturally occurring plant cells. This transgenic plant cell can then be cultured much like bacteria and yeast cells using modern microbiology techniques and may exist in an undifferentiated, unicellular state. The new plant cell's genetic composition and phenotype is a technical effect created by the integration of a heterologous DNA into the genome of the cell.

Provided are methods of using a plant cell, such as transgenic plant cells. These include (i) methods of producing transgenic cells by integrating a recombinant DNA into the genome of the cell and then using this cell to derive additional cells possessing the same heterologous DNA; (ii) methods of culturing cells that contain recombinant DNA using modern microbiology techniques; (iii) methods of producing and purifying endogenous or recombinant carbohydrate, lipid, nucleic acid, enzymes or protein products from cultured cells; and (iv) methods of using modern plant tissue culture techniques with transgenic plant cells to produce transgenic plants or transgenic plant tissue cultures.

The cotton plants, progeny, seeds, cells, and plant parts of the present disclosure may contain one or more additional desirable trait(s) or transgenic event(s). Such desirable traits may be transgenic traits, native traits, or mutant or edited traits or alleles produced by other methods such as genome editing, base editing, prime editing or other conventional mutagenesis methods. Desirable traits may be combined with cotton event Gh_CSM63718 by, for example, crossing a cotton plant comprising cotton event Gh_CSM63718 with another cotton plant containing the additional trait(s), or transgenic events. Such traits or transgenic events include, but are not limited to, increased insect resistance, increased water use efficiency, increased yield performance, increased drought resistance, increased disease resistance, increased seed or fiber quality, improved nutritional quality, hybrid seed production, and/or increase herbicide tolerance, in which the trait is measured with respect to a cotton plant lacking such transgenic trait. Many cotton transgenic events are known to those of skill in the art. For example, a list of such traits is provided by the United States Department of Agriculture's (USDA) Animal and Plant Health Inspection Service (APHIS) and can be found on their website www.aphis.usda.gov on the worldwide web. For example, any of the cotton plants, plant seeds, plant parts, or plant cells of the present disclosure can further comprise additional transgenes selected from the group consisting of Cry1B.3, Cry1 Da_7, Vip3Cb1.1, Cry2Ab, and combinations of any thereof. An illustrative Cry1B.3 coding sequence and its corresponding amino acid sequence are provided as SEQ ID NO:224 and SEQ ID NO:225, respectively. An illustrative Cry1 Da_7 coding sequence and its corresponding amino acid sequence are provided as SEQ ID NO:226 and SEQ ID NO:227, respectively. An illustrative Vip3Cb1.1 coding sequence and its corresponding amino acid sequence are provided as SEQ ID NO:228 and SEQ ID NO:229, respectively. An illustrative Cry2Ab coding sequence and its corresponding amino acid sequence are provided as SEQ ID NO:230 and SEQ ID NO:231, respectively. For example, the cotton plant, plant seed, plant part, or plant cell can further comprise cotton event Gh_BCS246002 and/or cotton event MON15947. The genetic elements in the transgenic inserts of cotton event Gh_BCS246002 and cotton event MON15947 are described further hereinbelow. These additional transgenes can provide cotton plants resistance to infestations by Lepidopteran pests such as Cotton Bollworm (Helicoverpa zea), Tobacco Budworm (Heliothis virescens), Fall Armyworm (Spodoptera frugiperda), Old World Bollworm (Helicoverpa armigera), and any combination of any two or three or all four of the foregoing pest species.

Alternatively or in addition, the cotton Gh_CSM63718 event can be stacked by breeding or by retransformation or by site directed integration/introgression with other events or combinations of events known in the art including, but not limited to:
19-51a (DD-01951A-7 for herbicide tolerance, described in USDA-APHIS Petition 95-256-01p, the entire contents and disclosure of which are incorporated herein by reference in their entirety),
281-24-236 (also known as DAS-24236-5 for herbicide tolerance and insect resistance, deposited as ATCC PTA-6233 and described in U.S. Pat. Nos. 7,179,965 and 7,883,850, the entire contents and disclosure of which are incorporated herein by reference in their entirety),
3006-210-23 (also known as DAS-21023-5 for herbicide tolerance and insect resistance, deposited as ATCC PTA-6233, and described in U.S. Pat. Nos. 7,179,965 and 7,883,850, the entire contents and disclosure of each of which are incorporated herein by reference in their entirety),
31707, 31803, 31807, 31808 and 42317 (also known as BXN™ Plus Bollgard™ Cotton for herbicide tolerance and insect resistance, described in USDA-APHIS Petition 97-013-01p, the entire contents and disclosure of which are incorporated herein by reference in their entirety),
BXN10211 (also known as 10211, BXN-10211-9, BXN™ Cotton for herbicide tolerance, described in USDA-APHIS Petition 93-196-01p, the entire contents and disclosure of which are incorporated herein by reference in their entirety),
BXN10215 (also known as 10215, BXN-10215-4, BXN™ Cotton for herbicide tolerance, described in USDA-APHIS Petition 93-196-01p, the entire contents and disclosure of which are incorporated herein by reference in their entirety),
BXN10222 (also known as 10222, BXN-10222-2, BXN™ Cotton for herbicide tolerance, described in USDA-APHIS Petition 93-196-01p, the entire contents and disclosure of which are incorporated herein by reference in their entirety),
BXN10224 (also known as IR102, for herbicide tolerance, described in USDA-APHIS Petition 93-196-01p, the entire contents and disclosure of which are incorporated herein by reference in their entirety),
COT102 (also known as IR102, SYN-IR102-7 for insect resistance, described in U.S. Pat. No. 7,371,940, and in USDA-APHIS Petition 03-155-01p, the entire contents and disclosure of which are incorporated herein by reference in their entirety),
COT202 (for insect resistance, described in U.S. Pat. No. 7,521,550, the entire contents and disclosure of which are incorporated herein by reference in their entirety),
COT203 (for insect resistance, described in PCT Patent Publication No. WO2005054480, the entire contents and disclosure of which are incorporated herein by reference in their entirety),
COT67B (also known as IR67B, SYN-IR67ß-1 for insect resistance, described in USDA-APHIS Petition 07-108-01p, the entire contents and disclosure of which are incorporated herein by reference in their entirety),
1143-14A (for insect resistance, described in PCT Patent Publication No. WO2006128570, the entire contents and disclosure of which are incorporated herein by reference in their entirety),
1143-51B (for insect resistance, described in PCT Patent Publication No. WO2006128569, the entire contents and disclosure of which are incorporated herein by reference in their entirety),
CE43-67B (for insect resistance, described in U.S. Pat. Nos. 7,834,254 and 9,131,651, the entire contents and disclosure of each of which are incorporated herein by reference in their entirety),
CE44-69D (for insect resistance, described in US Patent Publication No. 20100024077, the entire contents and disclosure of which are incorporated herein by reference in their entirety),
CE46-02A (for insect resistance, described in PCT Patent Publication No. WO2006128572, the entire contents and disclosure of which are incorporated herein by reference in their entirety),
DAS81910 (also known as DAS-81910-7 for herbicide tolerance, deposited as ATCC PTA-12456, and described in U.S. Pat. Nos. 9,551,024 and 9,896,718, and in USDA-APHIS Petition 13-262-01p, the entire contents and disclosure of each of which are incorporated herein by reference in their entirety),
GHB119 (also known as BCS-GH005-8, EE1-H6 for insect resistance, deposited as ATCC PTA-8398, and described in U.S. Pat. Nos. 8,309,818 and 9,328,390, the entire contents and disclosure of each of which are incorporated herein by reference in their entirety),
GHB614 (also known as BCS-GH002-5, EE-GH3, and GlyTofl™ for herbicide tolerance, deposited as ATCC PTA-6878, and described in U.S. Pat. Nos. 7,932,439, 8,501,411 and 9,394,566, and in USDA-APHIS Petition 06-332-01p, the entire contents and disclosure of each of which are incorporated herein by reference in their entirety),
GHB811 (also known as BCS-GH811-4 for herbicide tolerance, described in USDA-APHIS Petition 17-138-01p, the entire contents and disclosure of which are incorporated herein by reference in their entirety),
LLCotton25 (also known as ACS-GH001-3, EE-GH1, and Fibermax™ Liberty Link™ for herbicide tolerance, deposited as ATCC PTA-3343, and described in U.S. Pat. Nos. 6,818,807, 7,442,504 and 7,834,168, the entire contents and disclosure of each of which are incorporated herein by reference in their entirety),
T303-3 (also known as BCS-GH003-6 for herbicide tolerance and insect resistance, described in USDA-APHIS Petition 12-033-01p (Extension of 08-340-01p), the entire contents and disclosure of which are incorporated herein by reference in their entirety),
T304-40 (also known as BCS-GH004-7 and EE-GH5 for herbicide tolerance and insect resistance, deposited as ATCC PTA-8171, and described in U.S. Pat. Nos. 8,247,654, 9,382,550 and 10,356,996, and in USDA-APHIS Petition 08-340-01p, the entire contents and disclosure of each of which are incorporated herein by reference in their entirety),
T342-142 (for insect resistance, described in PCT Patent Publication No WO2006128568, the entire contents and disclosure of which are incorporated herein by reference in their entirety),
MON1076 (also known as 1076, MON-89924-2 and Bollgard™ Cotton for insect resistance, described in USDA-APHIS Petition 94-308-01p, the entire contents and disclosure of which are incorporated herein by reference in their entirety),
MON1445 (also known as 1445, MON-01445-2, and Roundup Ready™ Cotton for herbicide tolerance, described in U.S. Pat. Nos. 6,740,488, 7,189,514, 7,807,357 and 7,820,392, and in USDA-APHIS Petition 95-045-01p, the entire contents and disclosure of each of which are incorporated herein by reference in their entirety),
MON15985 (also known as MON-15985-7, and Bollgard II™ Cotton for insect resistance, deposited as ATCC PTA-2516, described in U.S. Pat. Nos. 7,223,907, 7,858,764 and 9,133,473, and in USDA-APHIS Petition 00-342-01p, the entire contents and disclosure of each of which are incorporated herein by reference in their entirety),
MON15947 (for insect resistance, described in USDA-APHIS Confirmation Request 21-211-01cr and subsequent USDA confirmation, as well as U.S. Pat. Nos. 7,223,907, 7,858,764 and 9,133,473, the entire contents and disclosure of which are incorporated herein by reference in their entirety),
MON1698 (also known as MON-89383-1, and Roundup Ready™ Cotton for herbicide tolerance, described in USDA-APHIS Petition 95-045-01p, the entire contents and disclosure of which are incorporated herein by reference in their entirety),
MON531 (also known as MON-00531-6, and Bollgard™ Cotton, Ingard™ for insect tolerance, described in USDA-APHIS Petition 94-308-01p, the entire contents and disclosure of which are incorporated herein by reference in their entirety),
MON757 (also known as MON-00757-7, and Bollgard™ Cotton for insect tolerance, described in USDA-APHIS Petition 94-308-01p, the entire contents and disclosure of which are incorporated herein by reference in their entirety),
MON88701 (also known as MON-88701-3, and Dicamba-Glufosinate Tolerant Cotton for herbicide tolerance, deposited as ATCC PTA-11754, described in U.S. Pat. Nos. 8,735,661, 9,024,115, 10,030,277 and 10,774,341, and in USDA-APHIS Petition 12-185-01p, the entire contents and disclosure of each of which are incorporated herein by reference in their entirety),
MON88702 (also known as MON-88702-4 for insect resistance, deposited as ATCC PTA-122520, described in U.S. Pat. Nos. 10,604,769 and 11,286,499, the entire contents and disclosure of each of which are incorporated herein by reference in their entirety),
MON88913 (also known as MON-88913-8, and Roundup Ready™ Flex™ Cotton for herbicide tolerance, deposited as ATCC PTA-4854, described in U.S. Pat. Nos. 7,381,861, 8,071,735 and 8,435,743, and in USDA-APHIS Petition 04-086-01p, the entire contents and disclosure of each of which are incorporated herein by reference in their entirety),
TAM66274 (also known as TAM-66274-5 for modified product quality, deposited as ATCC PTA-124218, described in U.S. Pat. No. 10,604,764, and in USDA-APHIS Petition 17-292-01p, the entire contents and disclosure of each of which are incorporated herein by reference in their entirety),
281-24-236×3006-210-23 (also known as MXB-13, DAS-24236-5×DAS-21023-5, and WideStrike™ Cotton for insect resistance),
281-24-236×3006-210-23×COT102 (also known as DAS-24236-5×DAS-21023-5×SYN-IR102-7 for insect resistance),
281-24-236×3006-210-23×COT102×81910 (also known as DAS-24236-5×DAS-21023-5×SYN-IR102-7×DAS-81910-7 for insect resistance and herbicide tolerance),
3006-210-23×281-24-236×MON1445 (also known as DAS-21023-5×DAS-24236-5×MON-01445-2, and WideStrike™ Roundup Ready™ Cotton for insect resistance and herbicide tolerance),
3006-210-23×281-24-236×MON88913 (also known as DAS-21023-5×DAS-24236-5×MON-88913-8, and Widestrike™ Roundup Ready Flex™ Cotton for insect resistance and herbicide tolerance),
3006-210-23×281-24-236×MON88913×COT102 (also known as DAS-21023-5×DAS-24236-5×MON-88913-8×SYN-IR102-7, and Widestrike™×Roundup Ready Flex™ X VIPCOT™ Cotton for insect resistance and herbicide tolerance),
3006-210-23×281-24-236×MON88913×COT102×DAS81910 (also known as DAS-21023-5×DAS-24236-5×MON-88913-8×SYN-IR102-7×DAS-81910-7 for insect resistance and herbicide tolerance),
31807×31808 (for insect resistance and herbicide tolerance),
COT102×COT67B (also known as SYN-IR102-7×SYN-IR67ß-1 and VIPCOT™ Cotton for insect resistance),
COT102×COT67B×MON88913 (also known as SYN-IR102-7×SYN-IR67ß-1×MON-88913-8 and VIPCOT™ Roundup Ready Flex™ Cotton for insect resistance and herbicide tolerance),
COT102×MON15985 (also known as SYN-IR102-7×MON-15985-7 and Bollgard® III for insect resistance),
COT102×MON15985×MON88913 (also known as SYN-IR102-7×MON-15985-7×MON-88913-8 and Bollgard® III x Roundup Ready™ Flex™ for insect resistance and herbicide tolerance),
COT102×MON15985×MON88913×MON88701 (also known as SYN-IR102-7×MON-15985-7×MON-88913-8×MON88701-3 for insect resistance and herbicide tolerance),
GHB614×LLCotton25 (also known as BCS-GH002-5×ACS-GH001-3 and G1yTo1™ Liberty Link™ for herbicide tolerance),
GHB614×LLCotton25×MON15985 (also known as BCS-GH002-5×ACS-GH001-3×MON-15985-7 for insect resistance and herbicide tolerance),
GHB614×MON15985 (also known as BCS-GH002-5×MON-15985-7 for insect resistance and herbicide tolerance),
GHB614×T304-40×GHB119 (also known as BCS-GH002-5×BCS-GH004-7×BCS-GH005-8 and Glytol™ x Twinlink™ for insect resistance and herbicide tolerance),
GHB614×T304-40×GHB119×COT102 (also known as BCS-GH002-5×BCS-GH004-7×BCS-GH005-8×SYN-IR102-7 and GlyTol LibertyLink TwinLink® Plus for insect resistance and herbicide tolerance),
GHB811×T304-40×GHB119×COT102 (also known as BCS-GHB811-4×BCS-GH004-7×BCS-GH005-8×SYN-IR102-7 for insect resistance and herbicide tolerance),
LLCotton25×MON15985 (also known as ACS-GH001-3×MON-15985-7 and Fibermax™ Liberty Link™ Bollgard II™ for insect resistance and herbicide tolerance),
MON15985×MON1445 (also known as MON-15985-7×MON-01445-2 and Roundup Ready™ Bollgard II™ Cotton for insect resistance and herbicide tolerance),
MON531×MON1445 (also known as MON-00531-6×MON-01445-2 and Roundup Ready™ Bollgard™ Cotton for insect resistance and herbicide tolerance),
MON88701×MON88913 (also known as MON 88701-3×MON-88913-8 for herbicide tolerance),
MON88701×MON88913×MON15985 (also known as MON 88701-3×MON-88913-8×MON-15985-7 for insect resistance and herbicide tolerance),
MON88913×MON15985 (also known as MON-88913-8×MON-15985-7 and Roundup Ready™ Flex™ Bollgard II™ Cotton for insect resistance and herbicide tolerance),
T304-40×GHB119 (also known as BCS-GH004-7×BCS-GH005-8 and TwinLink™ Cotton for insect resistance and herbicide tolerance, described in in USDA-APHIS Petition 08-340-01p, the entire content and disclosure of which are incorporated herein by reference in their entirety),
T304-40×GHB119×COT102 (also known as BCS-GH004-7×BCS-GH005-8×SYN-IR102-7 for insect resistance and herbicide tolerance).

As used herein, “Gh_BCS246002” refers to cotton event Gh_BCS246002. Cotton seed comprising event Gh_BCS246002 has been deposited under ATCC Accession No. 127733 and is fully described and characterized in U.S. Patent Application No. 63/645,146, the entire contents and disclosure of which are incorporated herein by reference in their entirety. Transgenic cotton plants comprising cotton event Gh_BCS246002 comprise SEQ ID NO:212 (5′ cotton genomic flank sequence+transgenic insert+3′ cotton genomic flank sequence), SEQ ID NO:213 (transgenic insert), SEQ ID NOs:214-217 (5′ junction sequences), and SEQ ID NOs:218-221 (3′ junction sequences). The transgenic insert in cotton plants comprising event Gh_BCS246002 comprises three expression cassettes as shown in Table 2. In the first expression cassette, the DNA sequence encoding the Cry1B.3 protein in cotton event Gh_BCS246002 is operably linked to Cucumis melo Chlorophyl a/b binding protein promoter and leader (see, e.g., U.S. Pat. No. 10,550,401, referenced therein as SEQ ID NO:29) and a Medicago truncatula hypothetical protein 3′ UTR (or transcription termination sequence, see, e.g., U.S. Pat. No. 10,501,749, referenced therein as SEQ ID NO:3). In the second expression cassette, the DNA sequence encoding the Cry1 Da_7 protein in cotton event Gh_BCS246002 is operably linked to a synthetic promoter, leader, and intron (see, e.g., United States Patent Application Publication 2018/0216129 A1, referenced therein as SEQ ID NOs:5, 6, and 9) and a Medicago truncatula expansin-related protein 1 precursor protein 3′ UTR (or transcription termination sequence, see, e.g., United States Patent Application Publication 2014/0283200 A1, referenced therein as SEQ ID NO:7). In the third expression cassette, the DNA sequence encoding the Vip3Cb1.1 protein in cotton event Gh_BCS246002 is operably linked to an Arabidopsis thaliana Actin-2 promoter, leader, and intron, and a Medicago truncatula seed maturation protein PM21 3′ UTR (or transcription termination sequence, see, e.g., United States Patent Application Publication 2014/0283200 A1, referenced therein as SEQ ID NO:16). Expression (transcription into the mRNAs coding for the toxin amino acid sequences, and translation of the mRNAs into the toxin proteins) of the toxin proteins Cry1B.3, Cry1 Da_7, and Vip3Cb1.1 from their respective transgene cassettes is oriented in the same direction (head to tail/head to tail/head to tail). The open reading frames are provided in the event in the following order in the 5′ to 3′ direction: the ORF for Cry1B.3, the ORF for Cry1 Da_7, and the ORF for Vip3Cb1.1, and as specified above, these three ORFs are comprised within SEQ ID NO:212 and SEQ ID NO:213.

Expression of the Cry1B.3, Cry1 Da_7, and Vip3Cb1.1 insect inhibitory proteins in cotton event Gh_BCS246002 provides resistance to the larval forms of Lepidopteran insect pests including, but not limited to, Cotton Bollworm (Helicoverpa zea), Tobacco Budworm (Heliothis virescens), and Fall Armyworm (Spodoptera frugiperda).

TABLE 2
Transgenic cassettes and elements in cotton event Gh_BCS246002

| Element | Description |
|---|---|
| 5′ Flanking DNA | DNA sequence flanking the 5′ end of the transgenic insert. |
| B-AGRtu.right | DNA region from Agrobacterium tumefaciens containing the right border |
| | sequence. |
| P-CUCme.Cab1 | The promoter from a CAB1 (Chlorophyll A/B BINDING protein) gene |
| | from Cucumis melo (melon). |
| L-CUCme.Cab1 | The 5′ untranslated region from a CAB1 (Chlorophyll A/B BINDING |
| | protein) gene from Cucumis melo (melon). |
| Cry1B.3 | Coding sequence of a chimeric insect toxin comprised of domains 1 and 2 |
| | of Cry1Be, domain 3 of Cry1Ka, and a protoxin domain of Cry1Be. |
| T-Mt.AC139600v16 | The 3′ untranslated region of a putative protein from Medicago truncatula |
| | (barrel medic). |
| P-At.GSP571 | A promoter that has been designed from multiple promoter sequences from |
| | Arabidopsis thaliana (thale cress) to direct constitutive expression. |
| L-At.GSP571 | A 5′ untranslated region that has been designed from multiple 5′ |
| | untranslated region sequences from Arabidopsis thaliana (thale cress) to |
| | regulate expression. |
| I-At.GSI21 | An intron that has been derived from multiple intron sequences from |
| | Arabidopsis thaliana (thale cress) to regulate expression. |
| Cry1Da_7 | Coding sequence of a Cry1Da insect toxin with amino acid modifications to |
| | improve efficacy. |
| T-Mt.Expr1 | The 3′ untranslated region for a putative oxidoreductase (OXR) protein |
| | from Medicago truncatula (barrel medic). |
| P-At.Act2 | The promoter of the Actin 2 (Act 2) gene from Arabidopsis thaliana (thale |
| | cress). |
| L-At.Act2 | The 5′ untranslated region of the Actin 2 (Act 2) gene from Arabidopsis |
| | thaliana (thale cress). |
| I-At.Act2 | The first intron of the Actin 2 (Act 2) gene from Arabidopsis thaliana |
| | (thale cress). |
| Vip3Cb1.1 | Coding sequence of a VipCb1 insect toxin. |
| T-Mt.MP21 | The 3′ untranslated region for a seed maturation protein (MP21) from |
| | Medicago truncatula (barrel medic). |
| RS-P1.lox1 | A recognition sequence for a site-specific Cre-recombinase from |
| | Enterobacteria phage P1. |
| B-AGRtu.left | DNA region from Agrobacterium tumefaciens containing the left border |
| | sequence. |
| 3′ Flanking DNA | DNA sequence flanking the 3′ end of the transgenic insert. |

102061 As used herein, “MON15947” refers to cotton event MON15947, which corresponds to event MON15985 without MON531 for insect resistance, an event developed by ballistic methods into germplasm containing event MON531, and which was determined to be unlinked to MON531. Cotton seed comprising event MON15985 has been deposited under ATCC Accession No. PTA-2516 and is fully described and characterized in U.S. Pat. Nos. 7,223,907, 7,858,764 and 9,133,473, the entire contents and disclosure of which are incorporated herein by reference in their entirety. Transgenic cotton plants comprising cotton event MON15947 comprise SEQ ID NO:232 (a 2,267-nucleotide sequence representing 1,877 nucleotides of the 5′ flank region of the cotton genomic DNA and 390 nucleotides of the integrated transgene insert), and SEQ ID NO:233 (a 1,360-nucleotide sequence representing 349 nucleotides of the integrated transgene insert and 1,012 nucleotides of the 3′ flank region of the cotton genomic DNA). The transgenic insert in cotton plants comprising event MON15947 comprises a Cry2Ab expression cassette, comprising the cauliflower mosaic virus (CaMV) 35S promoter with a duplicated enhancer region (U.S. Pat. Nos. 5,530,196, 5,424,200, and 5,359,142) operably connected or linked to a petunia heat shock protein untranslated leader sequence (PetHSP70-leader) operably-connected or linked to the N-terminal chloroplast transit peptide from Arabidopsis thaliana EPSPS gene (AEPSPS/CTP2) (Van den Broeck et. al. 1985) operably connected or linked to a synthetic sequence encoding Cry2Ab protein (Widner and Whiteley, 1990) which is operably connected or linked to the 3′ nontranslated region of the nopaline synthase (NOS) gene fromAgrobacterium tumifaciens which terminates transcription and directs polyadenylation (Fraley et al. 1983). Cotton plants comprising event MON15985 confers resistance to Lepidopteran insect pests.

Any of the cotton plants, plant parts, seeds, cells, progeny or commodity products described herein that comprise cotton event Gh_CSM63718 can further comprise cotton event Gh_BCS46002 and/or MON15947.

Any of the cotton plants, plant parts, seeds, cells, progeny or commodity products described herein can further comprise a recombinant DNA molecule comprising a sequence selected from the group consisting of SEQ ID NO:212; SEQ ID NO:213; SEQ ID NO:214; SEQ ID NO:215 SEQ ID NO:216; SEQ ID NO:217; SEQ ID NO:218; SEQ ID NO:219; SEQ ID NO:220; SEQ ID NO:221; SEQ ID NO:222; SEQ ID NO:223; SEQ ID NO:232; SEQ ID NO:233; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:212 or the full length of SEQ ID NO:213, and a complete complement of any of the foregoing.

Plants comprising cotton event Gh_CSM63718 and cotton event Gh_BCS246002 and/or cotton event MON15947 can be made by any method known in the art. For example, Plants comprising cotton event Gh_CSM63718 and/or cotton event Gh_BCS246002 and/or cotton event MON15947 can be made by crossing a cotton plant comprising cotton event Gh_CSM63718 with a cotton plant comprising cotton event Gh_BCS246002 or cotton event MON15947 and selecting for progeny plants containing both Gh_CSM63718 and Gh_BCS246002 or selecting for progeny plants containing both Gh_CSM63718 and MON15947. Plants comprising cotton events Gh_CSM63718, Gh_BCS246002 and MON15947 can be made, for example, by crossing a cotton plant comprising event Gh_CSM63718 with a cotton plant comprising both Gh_BCS246002 and MON15947, or by crossing a cotton plant comprising event Gh_BCS246002 with a cotton plant comprising event MON15947 first, selecting for a progeny plant comprising both Gh_BCS246002 and MON15947; and then crossing the progeny plant comprising both Gh_BCS246002 and MON15947 with a plant comprising event Gh_CSM63718, and selecting for progeny plants comprising Gh_BCS246002, MON15947 and Gh_CSM63718. Alternatively, a cotton plant comprising cotton event Gh_CSM63718 can be crossed with a cotton plant comprising event Gh_BCS246002 (or event MON15947) first, selecting for a progeny plant comprising both cotton events Gh_CSM63718 and Gh_BCS246002 (or MON15947), and then crossing the progeny plant comprising both Gh_CSM63718 and Gh_BCS246002 (or MON15947) with a plant comprising event MON15947 (or Gh_BCS246002), and selecting for a progeny plant comprising all three events.

The term “site-specific nuclease” refers to any enzyme that can cleave a nucleotide sequence in a site-specific manner. Site-specific nucleases allow for the precise and/or targeted editing of a specific location in a genome of a plant. Site-specific nucleases include, for example, RNA guided nucleases, zinc-finger nucleases (ZFNs), and transcription activator-like effector nucleases (TALENs).

Some site-specific nucleases, such as zinc finger nucleases (ZFNs) and TALENs, are not RNA-guided and instead rely on their protein structure to determine their target site for causing a DSB (double stranded break) or nick, or they are fused, tethered or attached to a DNA-binding protein domain or motif. The protein structure of the site-specific nuclease (or the fused/attached/tethered DNA binding domain) targets the site-specific nuclease to the target site. ZFNs, and TALENs, may be designed, engineered, and constructed according to known methods to target and bind to a target site.

RNA-guided nucleases are nucleases that form a complex (e.g., a ribonucleoprotein) with a guide RNA, which then guides the complex to a target site within a target sequence. One non-limiting example of guided nucleases are CRISPR nucleases. CRISPR (Clustered Regularly Interspaced Short Palindromic Repeats) nucleases are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells. CRISPR editing systems comprising a CRISPR associated protein (nuclease) and cognate guide RNAs (that can be transcribed from guide DNA polynucleotides) may be used for targeted DNA cleavage or modification. The CRISPR-associated protein can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7.

As used herein, the term “target site,” “genomic target site,” “target genomic nucleic acid,” or “target cotton genomic nucleic acid,” refers to a polynucleotide sequence that is sufficiently unique in the cotton genome to allow targeted genome modification by a site-specific nuclease. In one aspect, the sequence of the target site is changed from the wildtype sequence, namely the target site is edited. In another aspect, the target site is the site of insertion of a DNA sequence of interest.

The target site can comprise one or more of the criteria selected from the group consisting of: (i) the target site selected is more than 2 kb from a gene (ii) the target site selected is more than 1000 nucleotides (nt) from a small RNA hotspot, and (iii) the target site comprises 700 nucleotides of unique sequence on either side, Target site selection criteria are further described in US Patent Application Publication No. 2020/0024610 and U.S. Provisional Patent Application No. 63/387,871, the entire contents and disclosure of which are incorporated herein by reference in their entirety.

The target site comprises a sequence that is recognized by a site-specific nuclease. In some embodiments, the target site comprises a sequence that is recognized by a site-specific nuclease resulting in precise or targeted cleavage within the target site. For example, the site-specific nuclease can be selected from the group consisting of an RNA-guided nuclease, a Zinc Finger nuclease, and a TALEN. In some embodiments, the target site comprises a PAM (Protospacer Adjacent Motif) sequence that is recognized by an RNA-guided nuclease (e.g., a CRISPR nuclease system). For example, the target site can comprise a PAM motif that is recognized by a Cas12a/Cpf1 CRISPR nuclease system. The target site can further comprise a sequence that is recognized by and hybridizes to a CRISPR guide RNA. In some embodiments, the target site comprises a sequence that is recognized by and hybridizes to a Cas12a/Cpf1 CRISPR guide RNA.

A DNA sequence of interest can be inserted at a target site using a site-specific nuclease. As used herein, the term “DNA sequence of interest” or “donor sequence” or “donor DNA” refers to a nucleic acid/DNA sequence that has been selected for targeted insertion into a cotton genomic sequence. In one aspect, the cotton genomic sequence is a genomic target site described above. A DNA sequence on interest can be of any length, for example between 2 and 50,000 nucleotides in length (or any integer value therebetween). In some embodiments, the DNA sequence is between about 1,000 and 5,000 nucleotides in length (or any integer value therebetween). In some embodiments, the DNA sequence is between about 5,000 and 10,000 nucleotides in length (or any integer value therebetween). In some embodiments, the DNA sequence is between about 10,000 and 15,000 nucleotides in length (or any integer value therebetween). In some embodiments, the DNA sequence is between about 15,000 and 20,000 nucleotides in length (or any integer value therebetween). In some embodiments, the DNA sequence is between about 20,000 and 25,000 nucleotides in length (or any integer value therebetween). In some embodiments, the DNA sequence is between about 25,000 and 30,000 nucleotides in length (or any integer value therebetween). In some embodiments, the DNA sequence is between about 30,000 and 35,000 nucleotides in length (or any integer value therebetween). In some embodiments, the DNA sequence is between about 35,000 and 40,000 nucleotides in length (or any integer value therebetween). In some embodiments, the DNA sequence is between about 40,000 and 45,000 nucleotides in length (or any integer value therebetween). In some embodiments, the DNA sequence is between about 45,000 and 50,000 nucleotides in length (or any integer value therebetween). A DNA sequence may comprise one or more gene expression cassettes that further comprise actively transcribed and/or translated gene sequences. For example, the DNA sequence of interest can comprise a gene expression cassette comprising a sequence selected from: an herbicide tolerance gene, an insecticidal resistance gene, a nitrogen use efficiency gene, a water use efficiency gene, a nutritional quality gene, a DNA binding gene, a selectable marker gene, a target site for a site-specific nuclease, and any combination thereof. Alternatively, the DNA sequence of interest may comprise a polynucleotide sequence which does not comprise a functional gene expression cassette or an entire gene (e.g., may comprise regulatory sequences such as promoters, enhancers, etc.), or may not contain any identifiable gene expression elements or any actively transcribed gene sequence. In some embodiments, the DNA of interest may comprise a cognate guide RNA recognition site (CgRRS). In some embodiments, the DNA of interest will have at least one homology arm DNA sequence. The term “homology arm DNA sequence” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell. Further, the DNA sequence can be linear or circular, and can be single-stranded or double-stranded. It can be delivered to the cell as naked nucleic acid, as a complex with one or more delivery agents (e.g., liposomes, poloxamers, T-strand encapsulated with proteins, etc.) or contained in a bacterial or viral delivery vehicle, such as, for example, Agrobacterium tumefaciens or a Gemini Virus, or a nanovirus, respectively.

Once a specific target site is identified, a site-specific nuclease targeting the selected target site can be designed and introduced into the plant, seed, or plant cell. For example, a CRISPR-associated nuclease (e.g., Cas12a/Cpf1) and at least one RNA guide molecule that can hybridize to the target site can be designed and cloned into a plant expression vector and delivered to the plant, seed, or plant cell. If the genome modification is designed to induce a double-strand break (DSB) (i.e., induce a cleavage) with non-homologous end joining (NHEJ) repair for introduction of insertions and deletions (indels), then just the engineered CRISPR nuclease and at least one RNA guide molecule is delivered to the plant, seed or cell. If a DNA sequence of interest is to be incorporated at the target site, then the engineered CRISPR nuclease, at least one RNA guide molecule, and the DNA of interest are co-delivered to the plant, seed, or cell. The DNA of interest may integrate into the target site by NHEJ (Non-homologous End Joining) or by homology-dependent repair (HR). In the latter case, the DNA of interest will have at least one homology arm DNA sequence. An alternative to delivery of the engineered CRISPR nuclease as a DNA expression construct is the delivery of a ribonucleoprotein (RNP) complex of the CRISPR associated nuclease protein in complex with the guide RNA.

Following delivery of the site-specific nuclease to a plant cell, the cells or plants regenerated from the cells are sampled to confirm the presence of the intended site-specific genome modification including insertion of the DNA sequence of interest at or proximal to the target site. Methods of detecting the genome modification are known to one skilled in the art, and include PCR, TaqMan® PCR, droplet digital PCR (ddPCR™, Bio-Rad Laboratories, Hercules, Calif.), sequencing, Sanger sequencing, ABI 3730 DNA fragment analysis (Applied Biosystems, Grand Island, N.Y.), Southern blot analysis, Northern blot analysis, phenotypic analysis, or any other technique known to one in the art to detect genome modification.

The plants described herein can be used to produce progeny or offspring that comprise cotton event Gh_CSM63718. Such progeny may include any plant, seed, and cell and/or regenerable plant part comprising cotton event Gh_CSM63718 inherited or derived from an ancestor or parental cotton plant(s), at least one of which comprises a DNA molecule having or comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 23 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9.

Cotton plants, progeny, and seeds may be homozygous or heterozygous for the event Gh_CSM63718 and the transgenes of event Gh_CSM63718. Progeny may be grown from seeds produced by a cotton plant comprising or containing event Gh_CSM63718 and/or from seeds produced by a plant fertilized with pollen from a cotton plant comprising or containing event Gh_CSM63718 (i.e., fertilized with pollen comprising or containing event Gh_CSM63718). Plants or progeny may also be obtained by tissue culture and regeneration methods from a protoplast, cell, embryo or reproductive or somatic tissue derived from a cotton plant comprising or containing cotton event Gh_CSM63718.

Progeny plants may be self-pollinated (also known as “selfing”) to generate a true breeding line of plants, i.e., plants homozygous for the cotton event Gh_CSM63718 DNA. Alternatively, progeny plants may be outcrossed, i.e., bred with another plant, to produce a varietal or a hybrid seed or plant. The other plant may be transgenic or non-transgenic. A varietal or hybrid seed or plant of the present disclosure may thus be derived by crossing a first parent that lacks the specific and unique DNA of event Gh_CSM63718 with a second parent comprising event Gh_CSM63718, resulting in a hybrid comprising the specific and unique DNA of event Gh_CSM63718. Each parent can be a hybrid or an inbred/variety, so long as the cross or breeding results in a plant or seed of the present disclosure, i.e., a seed having at least one allele comprising the specific and unique DNA of event Gh_CSM63718 and/or at least 16 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9.

Sexually crossing one plant with another plant, i.e., cross-pollinating, may be accomplished or facilitated by human intervention, for example: by human hands collecting the pollen of one plant and contacting this pollen with the style or stigma of a second plant; by human hands and/or human actions removing, destroying, or covering the stamen or anthers of a plant (e.g., by manual intervention or by application of a chemical gametocide) so that natural self-pollination is prevented and cross-pollination would have to take place in order for fertilization to occur; by human placement of pollinating insects in a position for “directed pollination” (e.g., by placing beehives in orchards or fields or by caging plants with pollinating insects); by human opening or removing of parts of the flower to allow for placement or contact of foreign pollen on the style or stigma; by selective placement of plants (e.g., intentionally planting plants in pollinating proximity); and/or by application of chemicals to precipitate flowering or to foster receptivity (of the stigma for pollen).

Two different transgenic plants of the same or different genetic backgrounds may thus be crossed to produce inbred or hybrid offspring plants, plant parts and/or seeds that contain two independently segregating transgenes or events wherein at least one of those transgenes or events comprises or is contained within cotton event Gh_CSM63718. For example, transgenic plants comprising cotton event Gh_CSM63718 can be crossed with other transgenic cotton plants to produce a plant having the characteristics of both transgenic parents.

Back-crossing to a parental plant and out-crossing with a non-transgenic plant are also contemplated, as is vegetative propagation. Descriptions of other breeding methods that are commonly used for different traits and crops are known in the art and can be found in one of several references, e.g., Fehr, in Breeding Methods for Cultivar Development, Wilcox J. ed., American Society of Agronomy, Madison WI (1987).

A plant part is provided. As used herein, a “plant part” refers to any part of a plant that is comprised of material directly from or derived from a plant comprising cotton event Gh_CSM63718. Plant parts include but are not limited to microspores, pollen, anthers, ovules, ovaries, flowers, bolls, embryos, buds, nodes, stems, leaves, roots, and calli, in whole or part. Plant parts may be viable or nonviable. Plant parts may be regenerable or non-regenerable.

Nonliving or nonregenerable cotton plant materials are provided herein. The nonliving or nonregenerable cotton plant material can comprise any of the recombinant DNA molecules characteristic of cotton event Gh_CSM63718 described herein, or any of the DNA constructs described herein. The nonliving or nonregenerable cotton plant material can comprise cotton event Gh_CSM63718, a representative sample of seed comprising the cotton event cotton event Gh_CSM63718 having been deposited under ATCC Accession No. PTA-127638.

Commodity products that comprise any of the DNA molecules characteristic of cotton event Gh_CSM63718 or any of the DNA constructs described herein are provided. Such commodity products can be produced from plants comprising cotton event Gh_CSM63718. The commodity products contain a detectable amount of DNA comprising a DNA sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. As used herein, a “commodity product” refers to any composition or product which is comprised of material from plant, seed, cell, or plant part comprising cotton event Gh_CSM63718. Commodity products may be viable or non-living plant material, that is, a material that is not living and derived from a plant, seed, cell, or plant part comprising cotton event Gh_CSM63718. Nonviable commodity products include but are not limited to nonviable seeds, whole or processed seeds, processed plant tissues or plant parts, dehydrated plant tissues or parts, frozen plant tissues or parts, food for human consumption such as cottonseed oil; plant parts processed for animal feed such as cottonseed cake, cottonseed meal and cottonseed hulls; cotton fiber and linters; plant parts processed into fertilizer, biochar and building boards such as cotton stalks. Viable commodity products include but are not limited to viable seeds, viable plant parts (such as pollen, ovule, ovary, flower, boll, embryo, node, bud, root and leaf) and viable plant cells. A plant comprising event Gh_CSM63718 can thus be used to manufacture any commodity product typically acquired from a cotton plant. Any such commodity product that is derived from the plants comprising event Gh_CSM63718 may contain at least a detectable amount of the specific and unique DNA corresponding to event Gh_CSM63718, and specifically may contain a detectable amount of a polynucleotide having a nucleotide sequence of at least 22 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. Any standard method of detection for polynucleotide molecules may be used, including methods of detection disclosed herein.

Methods for producing such commodity products are also provided. Such methods comprise: (a) obtaining a transgenic cotton plant, plant part, or plant seed comprising cotton event Gh_CSM63718; and (b) producing a commodity product from the transgenic cotton plant, plant part, or plant seed.

A plant tolerant to herbicides may be produced by sexually crossing a plant comprising event Gh_CSM63718 with another plant and thereby producing seed, which is then grown into progeny plants. For example, provided herein is a method of producing a progeny cotton plant comprising event Gh_CSM63718, the method comprising: (a) sexually crossing a first cotton plant that comprises cotton event Gh_CSM63718 with itself or a second cotton plant; (b) collecting one or more seeds produced from the cross; (c) growing one or more seeds to produce one or more progeny plants; and (d) selecting at least a first progeny plant or seed comprising cotton event Gh_CSM63718. Inbred and hybrid cotton plants comprising cotton event Gh_CSM63718 produced by such methods are also provided.

The progeny plants may be analyzed using diagnostic methods to select for progeny plants that comprise event Gh_CSM63718 DNA or for progeny plants tolerant to at least one herbicide selected from the group consisting of glufosinate, ß-triketone HPPD inhibitors, dicamba, glyphosate, PPO herbicides, and any combination thereof. Examples of ß-triketone HPPD inhibitors include mesotrione, benzobicyclon (BBC), tembotrione, sulcotrione, tefuryltrione, and any combination thereof. Examples of PPO herbicides include flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, trifludimoxazin, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate, and combinations of any thereof. The other plant used may or may not be transgenic. The progeny plant and/or seed produced may be varietal or hybrid seed.

A plant tolerant to glufosinate, ß-triketone HPPD inhibitor herbicides, dicamba, glyphosate and PPO herbicides may be produced by selfing a plant comprising event Gh_CSM63718 comprising a polynucleotide having the nucleotide sequence of SEQ ID NOs:1-10, at least 23 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9, or a complete complement of any of the foregoing, and thereby producing seed, which is then grown into progeny plants. These progeny plants may then be analyzed using diagnostic methods to select for progeny plants that comprise event Gh_CSM63718 DNA, or for progeny plants tolerant to the glufosinate, ß-triketone HPPD inhibitor herbicides, dicamba, glyphosate and PPO herbicides such as flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, trifludimoxazin, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate, and combinations of any thereof.

Cotton event Gh_CSM63718 contains the PAT, TDO, DMO, EPSPS and PPO expression cassettes that provides tolerance to glufosinate, ß-triketone HPPD inhibitor herbicides (such as mesotrione, benzobicyclon (BBC), tembotrione, sulcotrione, tefuryltrione, and any combination thereof), dicamba, glyphosate, and PPO herbicides such as flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate, and combinations of any thereof.

PPO herbicides include diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof. Examples of diphenylethers include, but are not limited to, acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof. Examples of N-phenylphthalimides include, but are not limited to, cinidon-ethyl, flumiclorac, flumiclorac-pentyl, and flumioxazin. Examples of oxadiazoles include, but are not limited to, oxadiargyl and oxadiazon. Examples of oxazolidinediones include, but are not limited to, pentoxazone. Examples of phenylpyrazoles include, but are not limited to, fluazolate, pyraflufen, and pyraflufen-ethyl. Examples of pyrimidinediones or phenyluracils include, but are not limited to, benzfendizone, butafenacil, epyrifencacil, flupropacil, flufenoximacil, saflufenacil, and tiafenacil. Examples of thiadiazoles include, but are not limited to, fluthiacet-methyl and thidiazimin. Examples of triazolinones include, but are not limited to, azafenidin, bencarbazone, carfentrazone, its salts and esters, and sulfentrazone. Examples of benzoxazinone derivatives include, but are not limited to, 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)). Examples of other PPO herbicides include, but are not limited to, chlorphthalim, flufenpyr, flufenpyr-ethyl, flumipropyn, pyraclonil, and profluazol. Further examples of other PPO herbicides include:

1) an herbicidally active compound of the general formula (I) or an agrochemically acceptable salt thereof

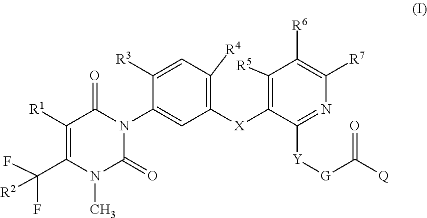

in which:
R¹ is hydrogen,
R² is hydrogen, fluorine, chlorine, bromine, trifluoromethyl, methoxy, ethoxy, prop-1-yloxy, or but-1-yloxy,
R³ is hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R⁴ is fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R⁵, R⁶ and R⁷ are independently hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, or 2,2,2-trifluoroethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-di-methylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, or 1-ethyl-2-methylpropyl-1-ene,
X and Y are independently O (oxygen) or S (sulfur)
and
Q is one of the following moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440:

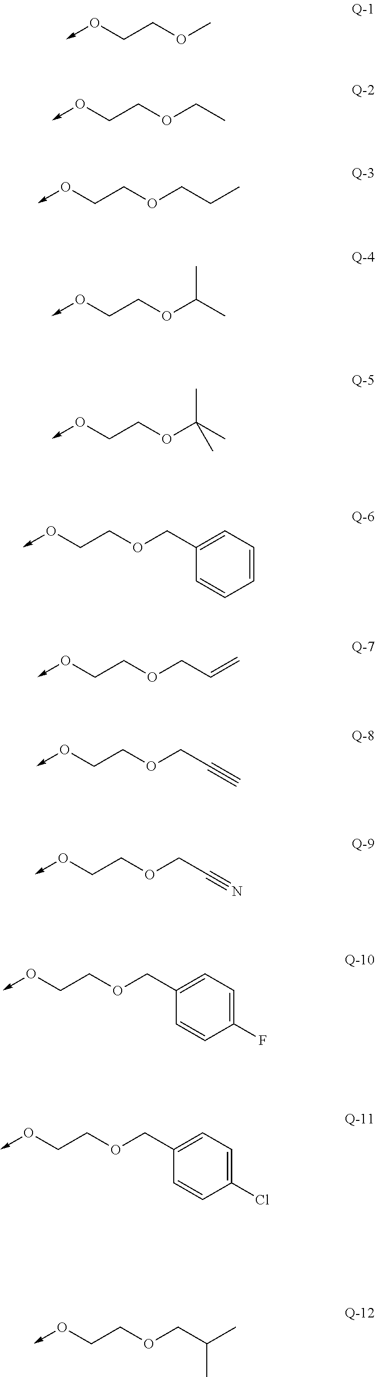

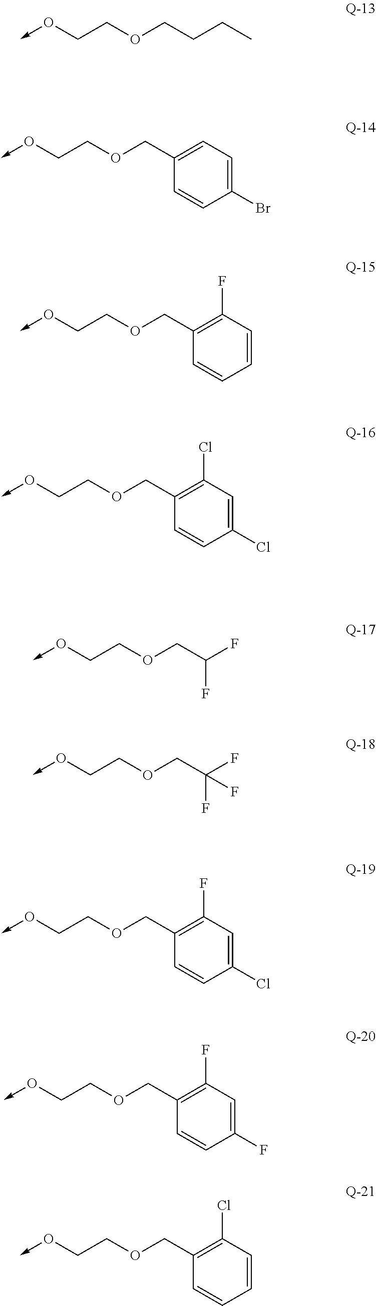

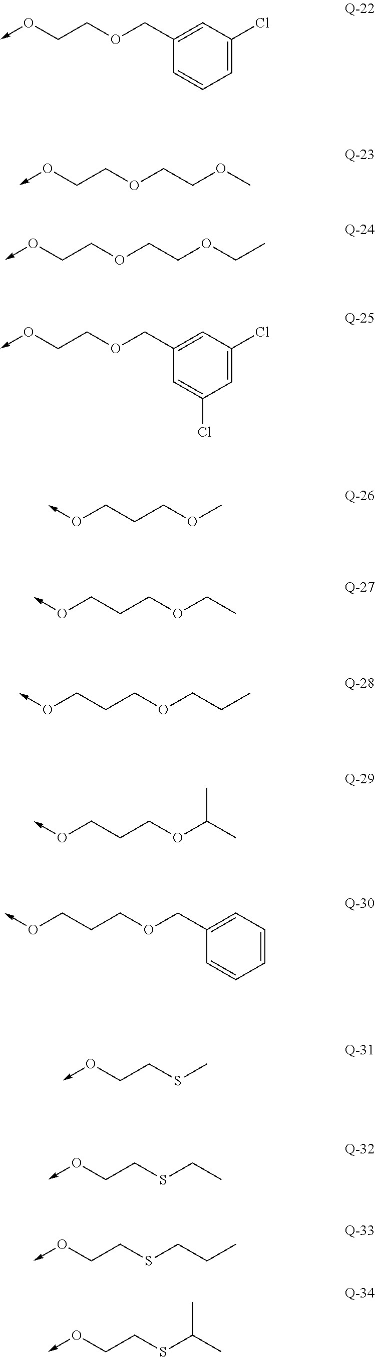

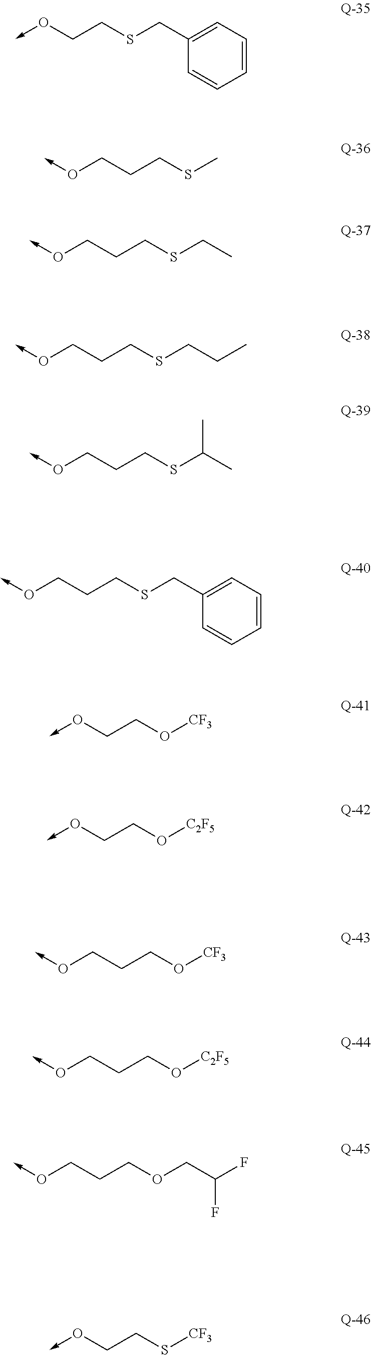

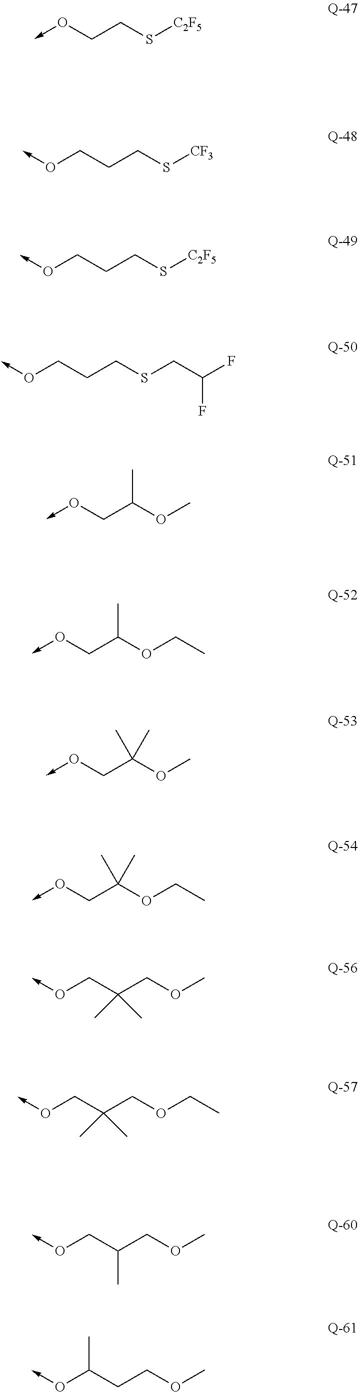

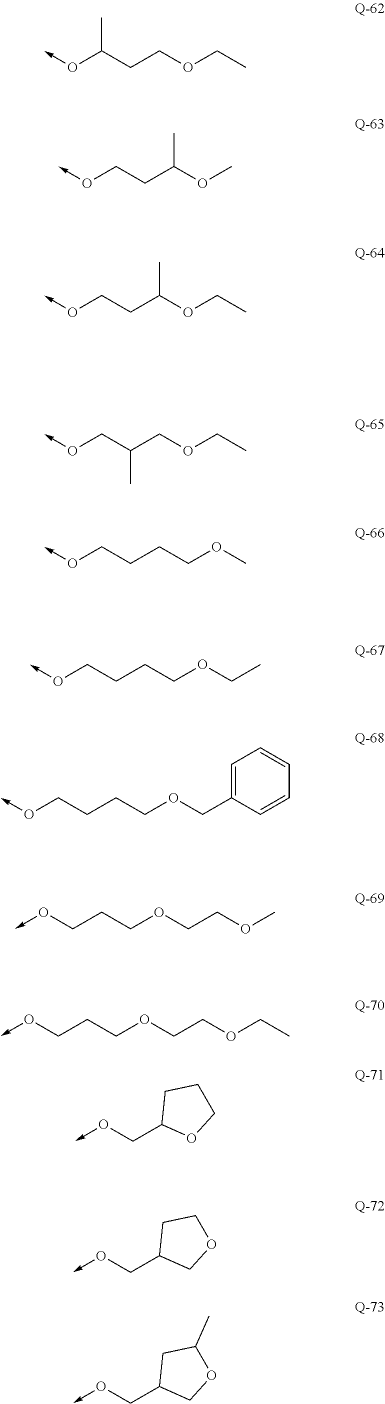

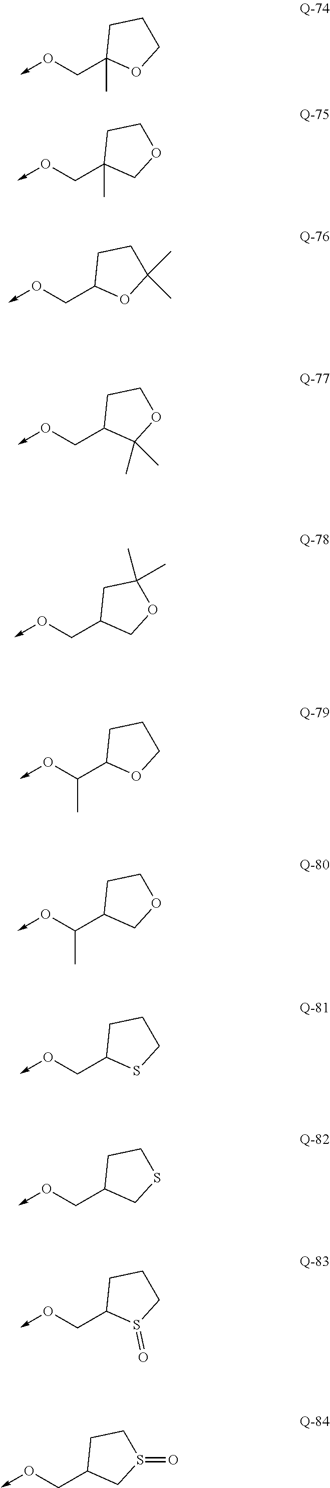

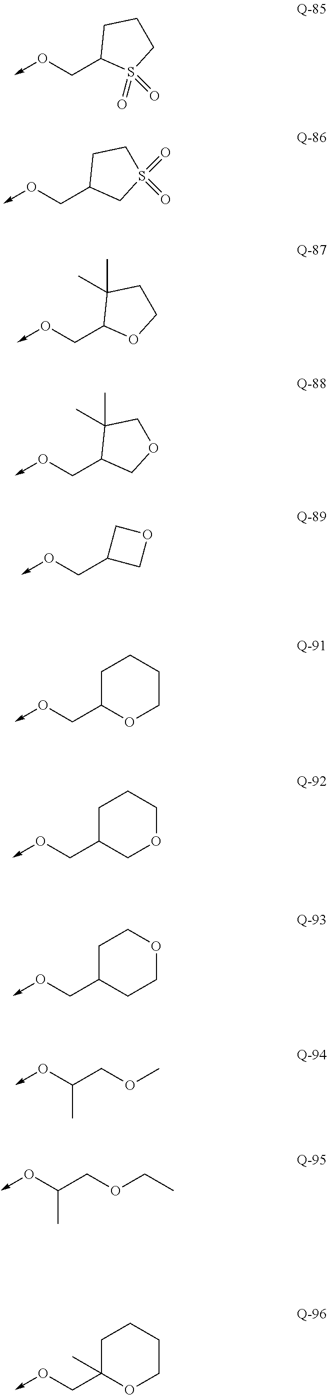

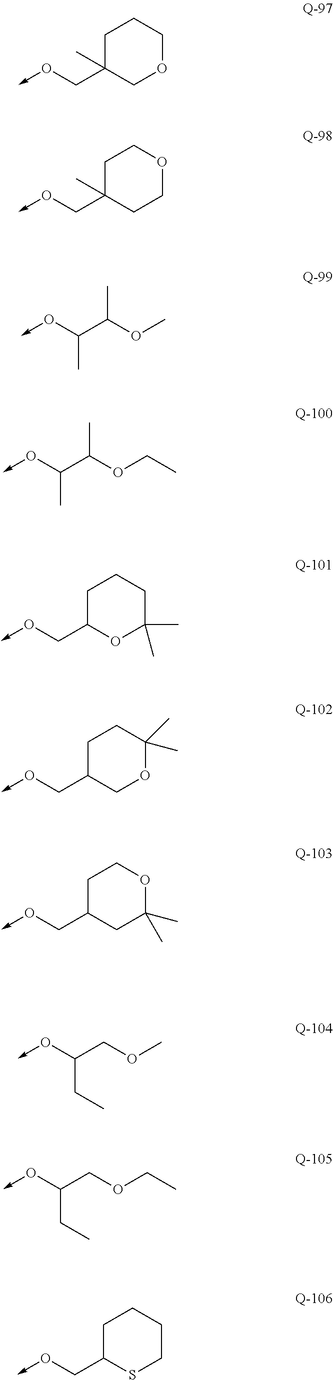

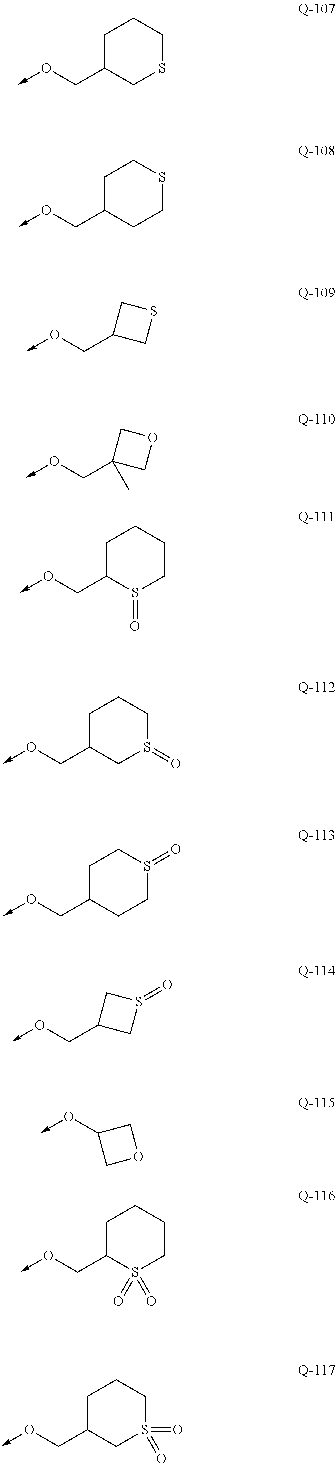

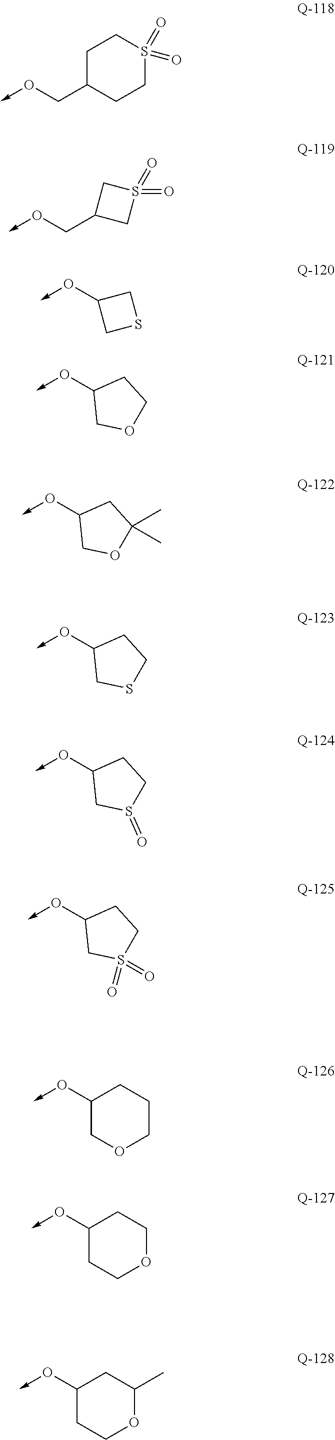

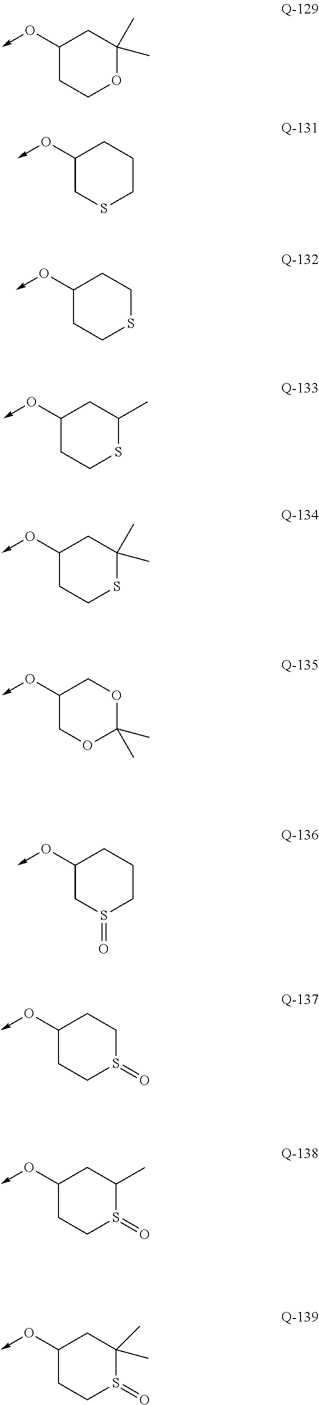

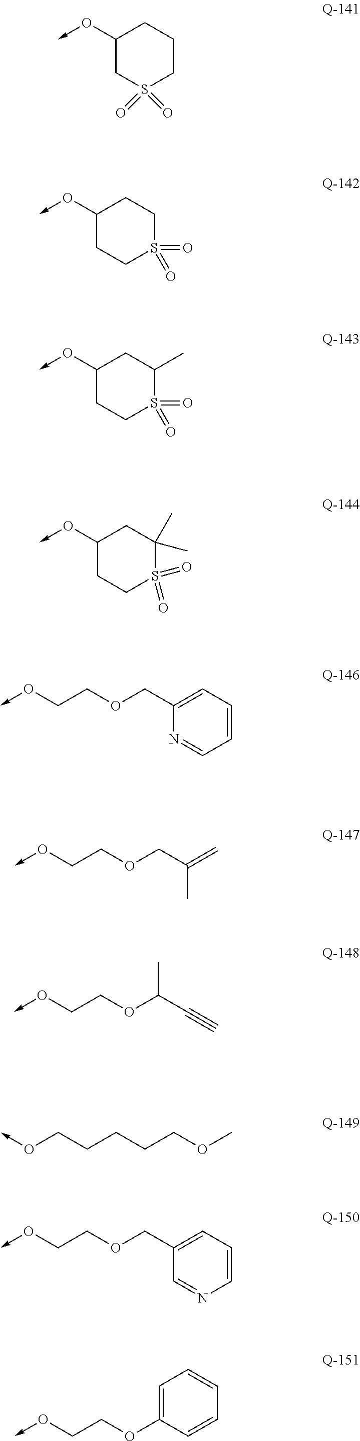

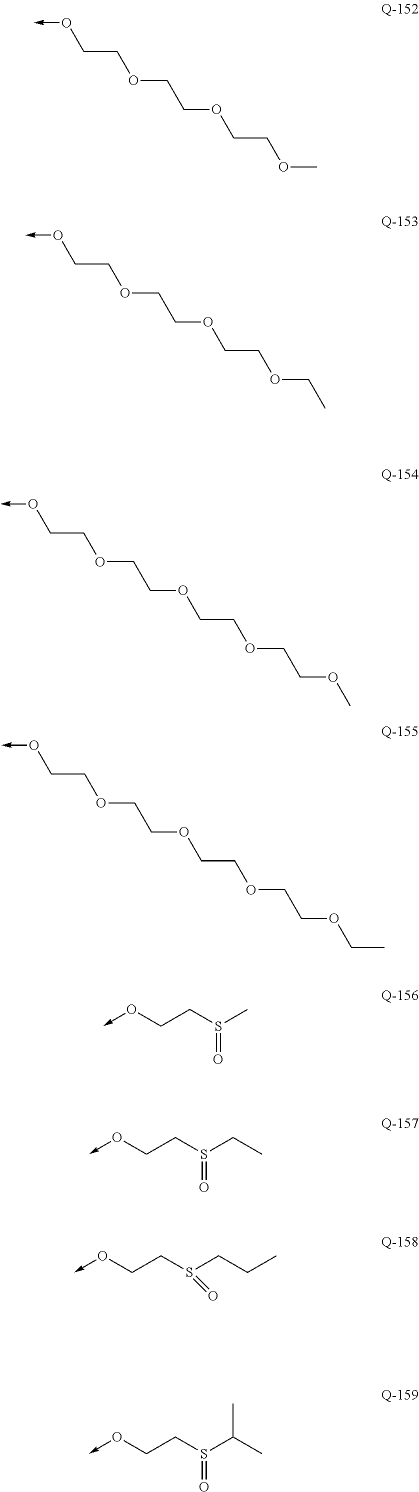

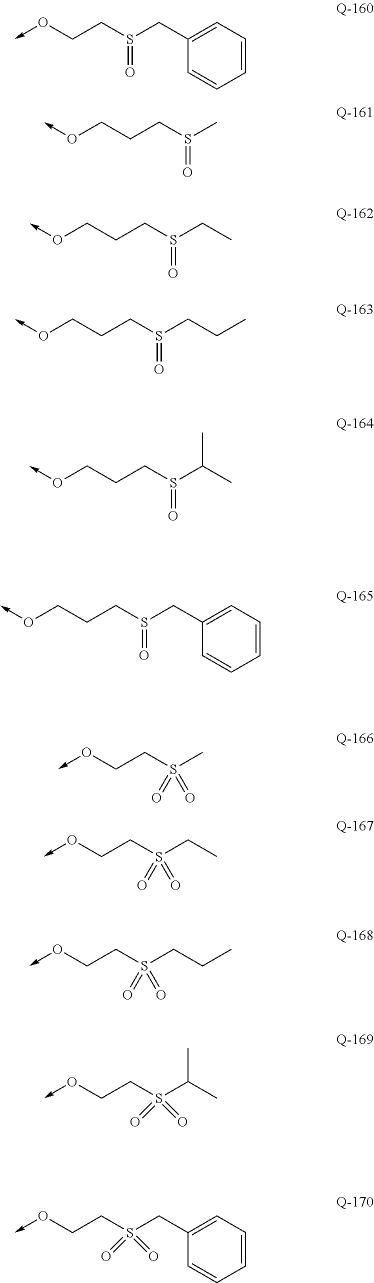

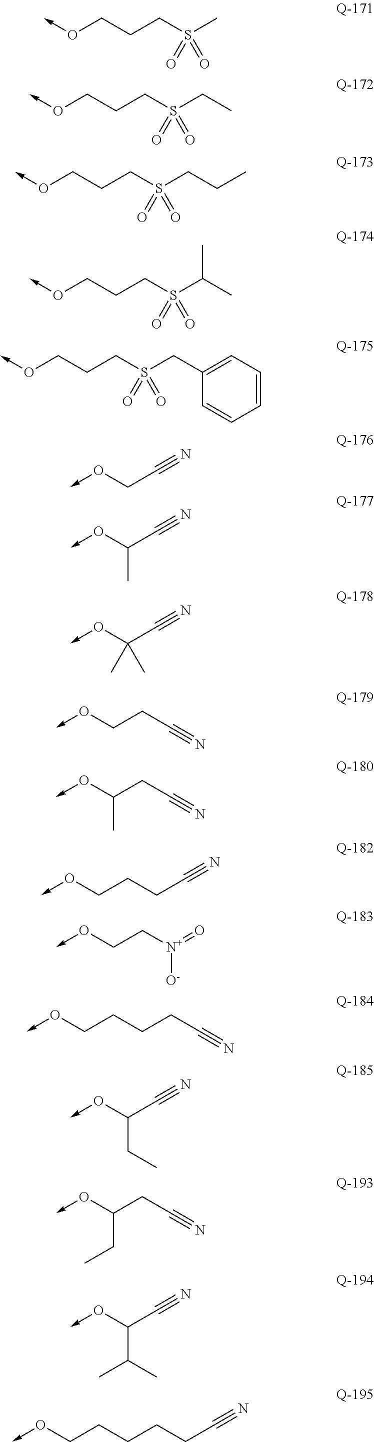

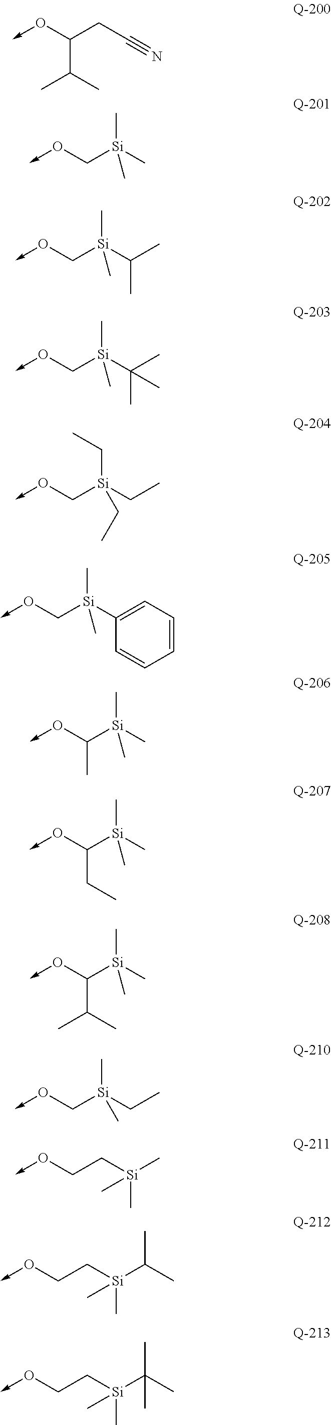

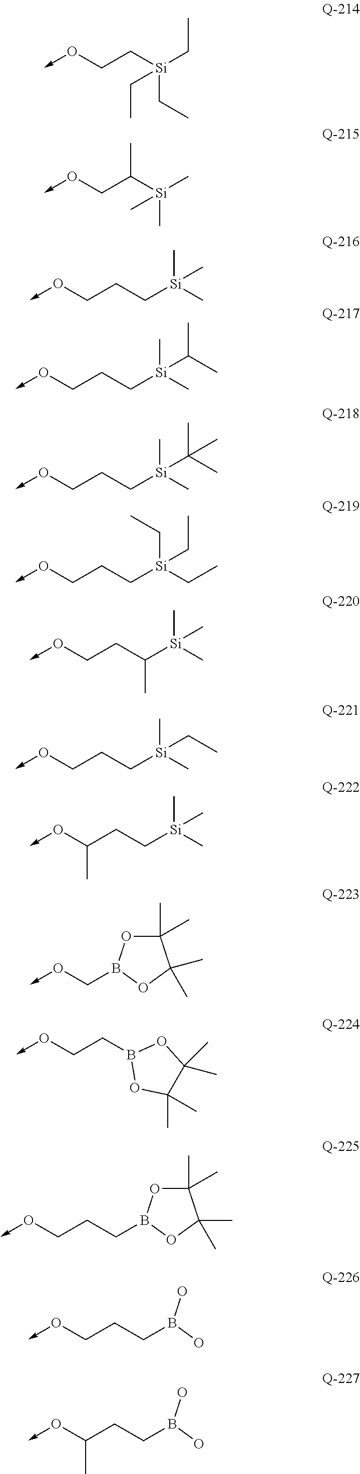

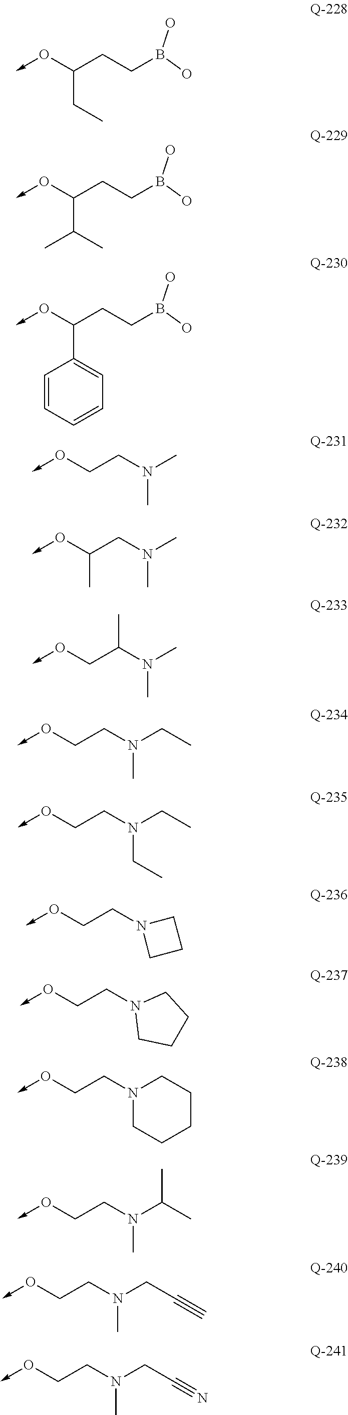

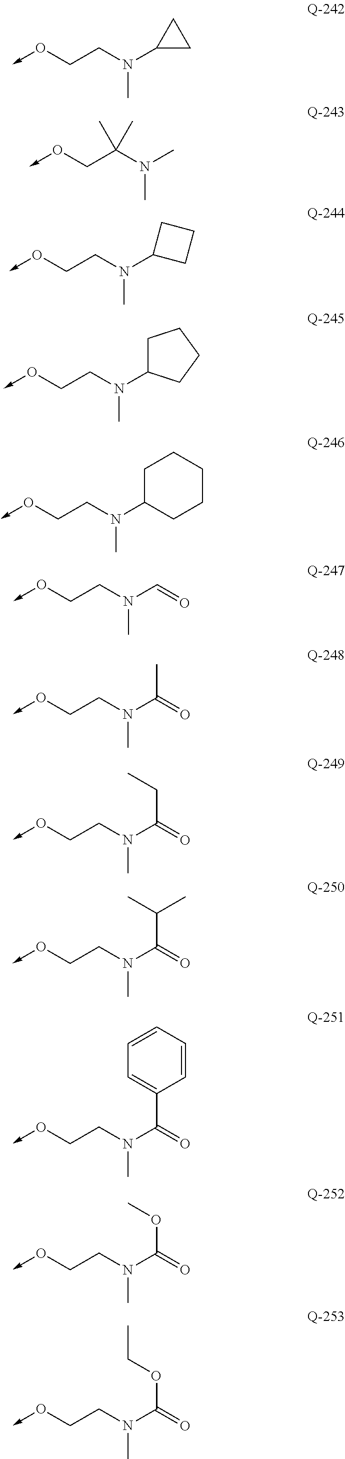

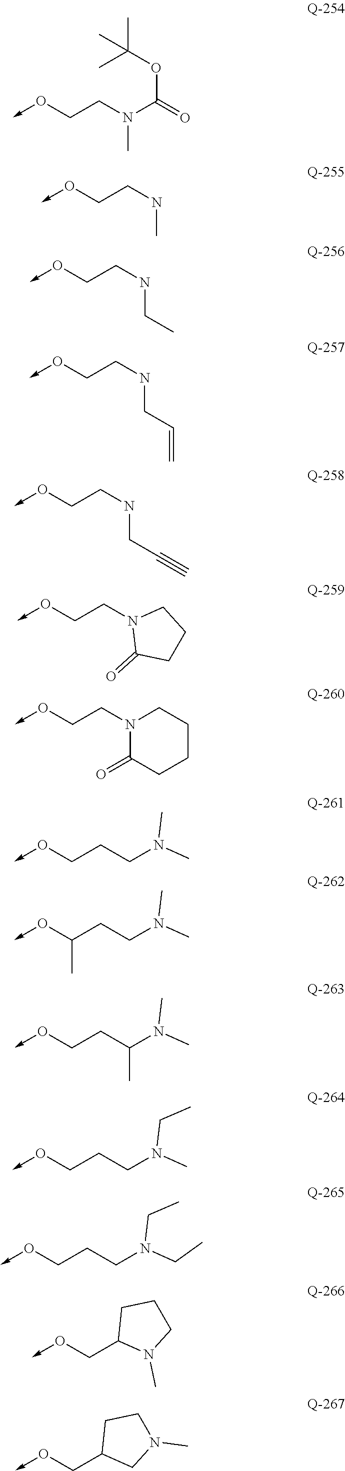

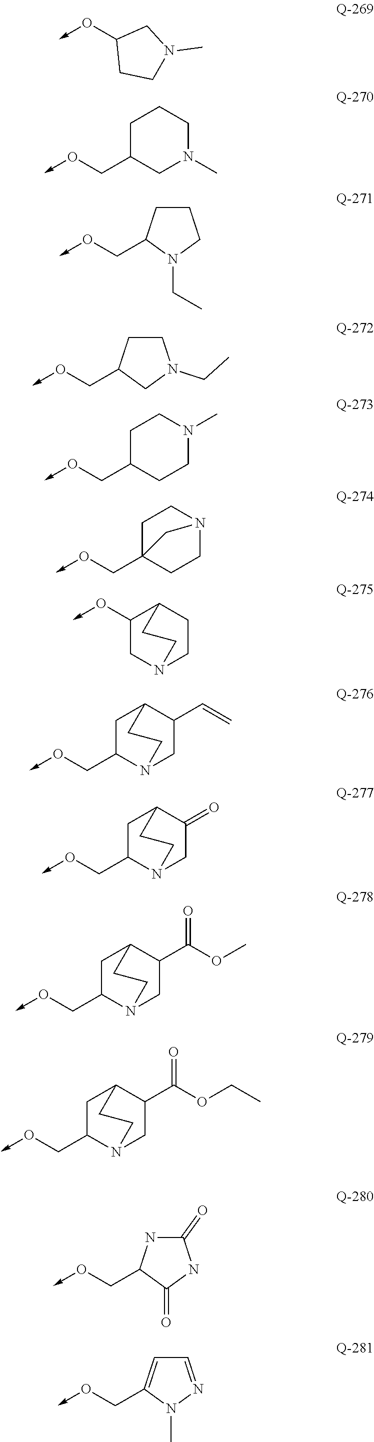

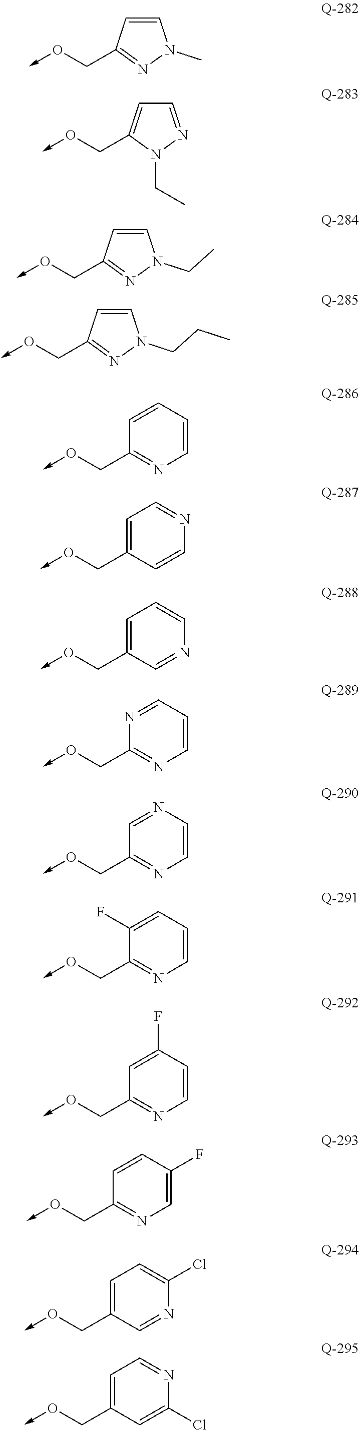

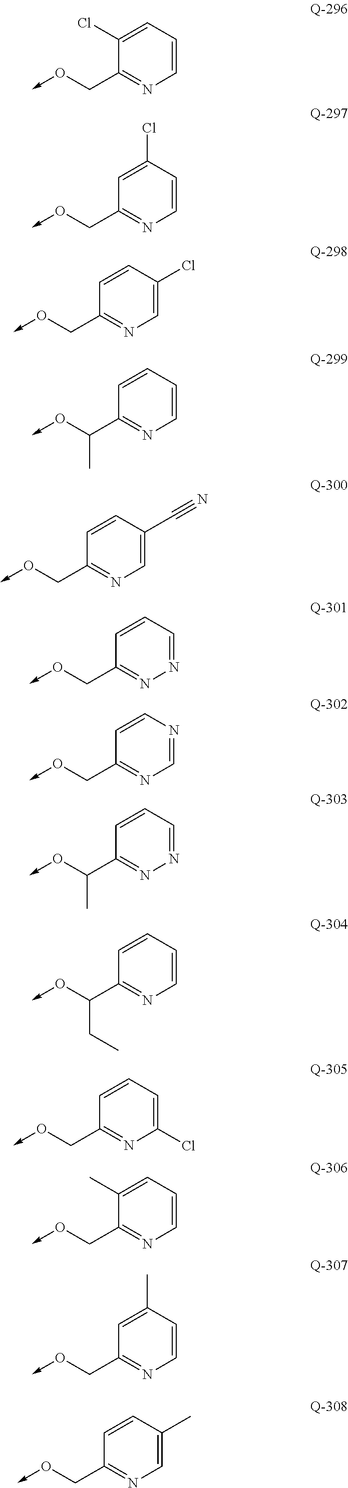

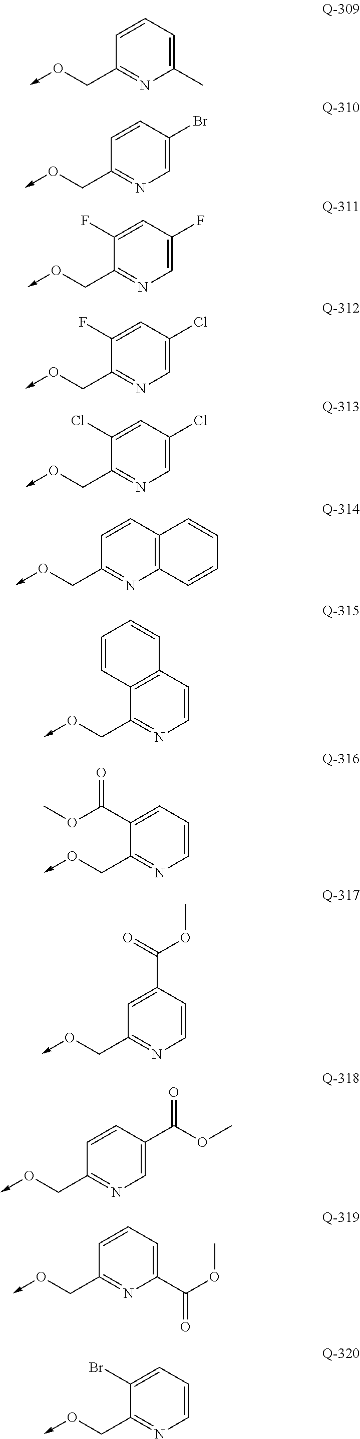

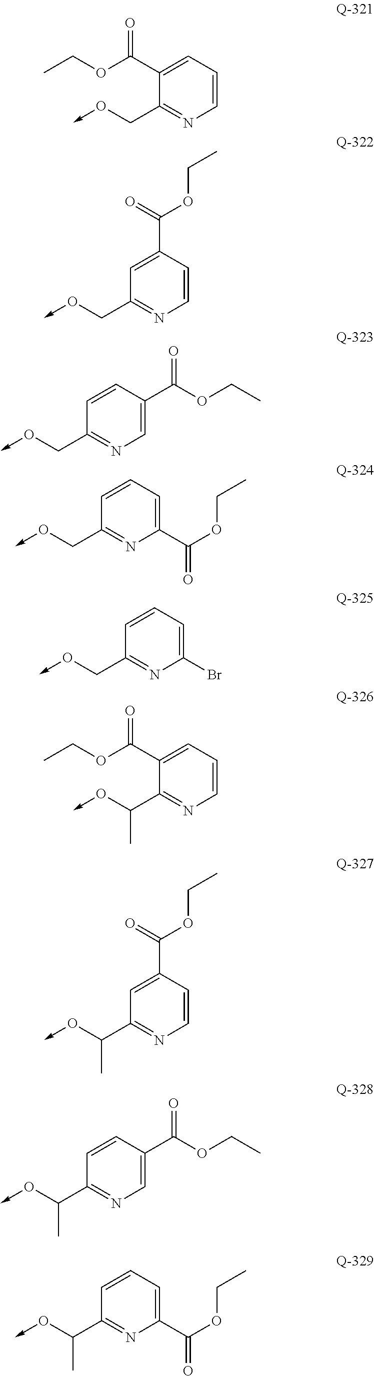

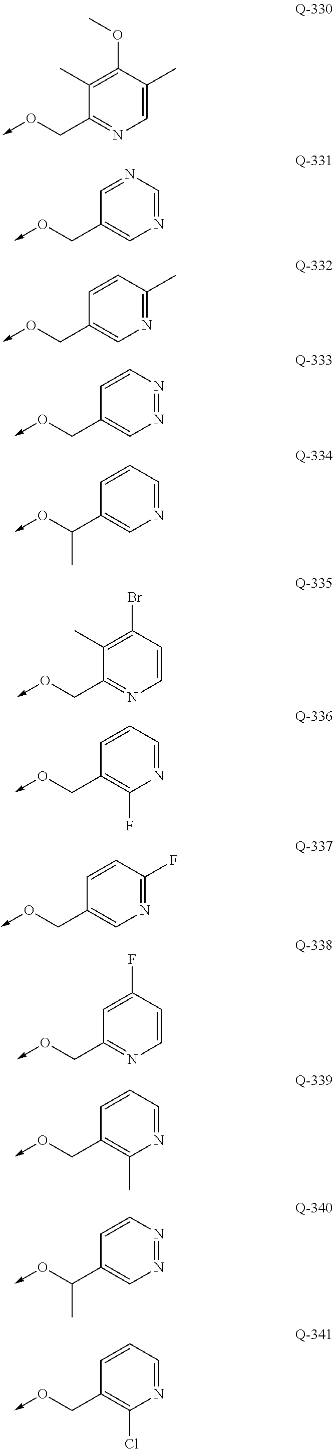

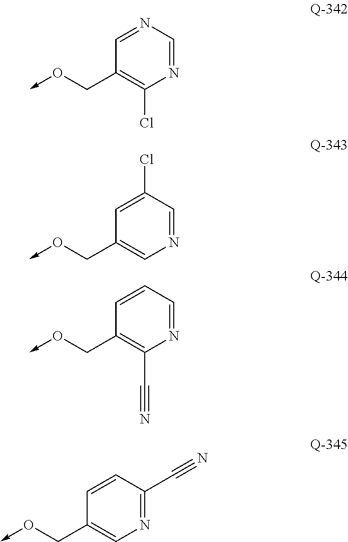

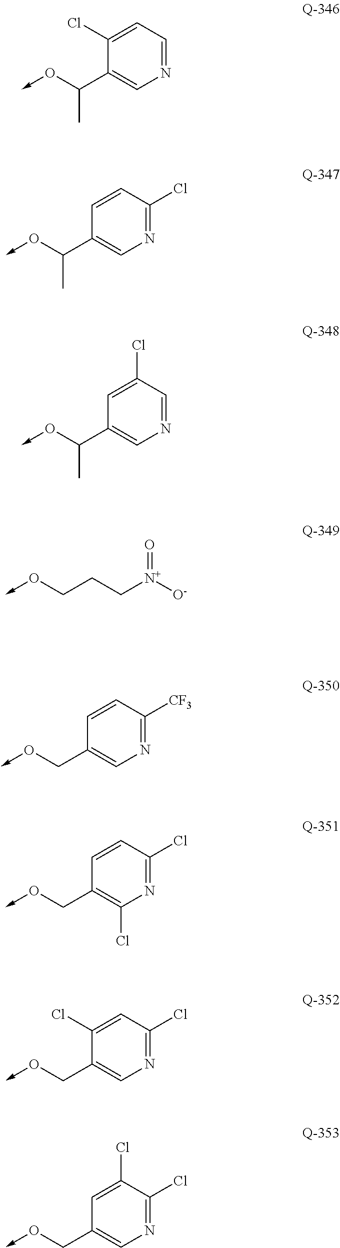

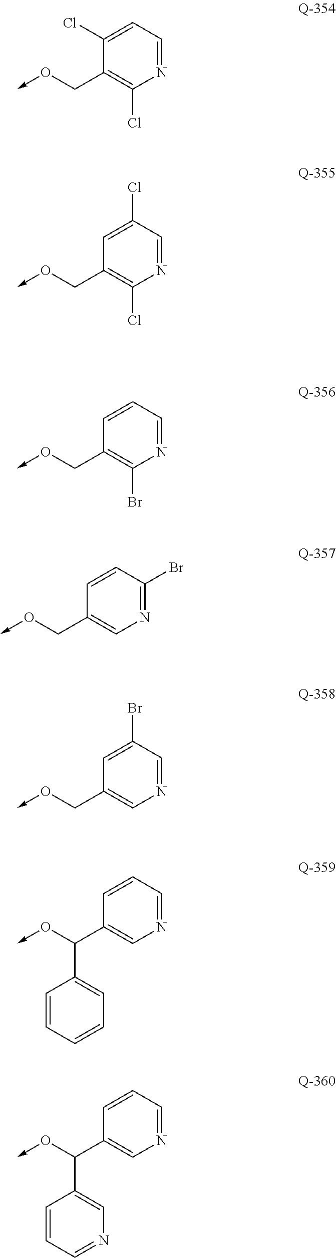

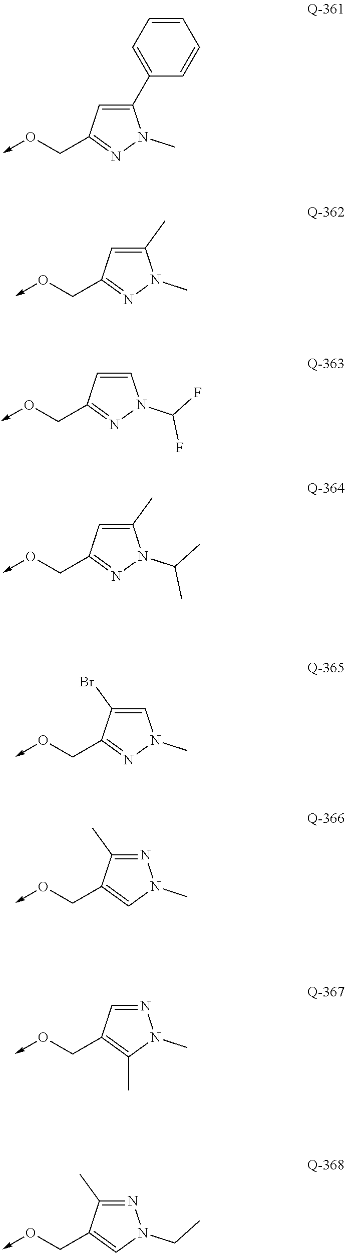

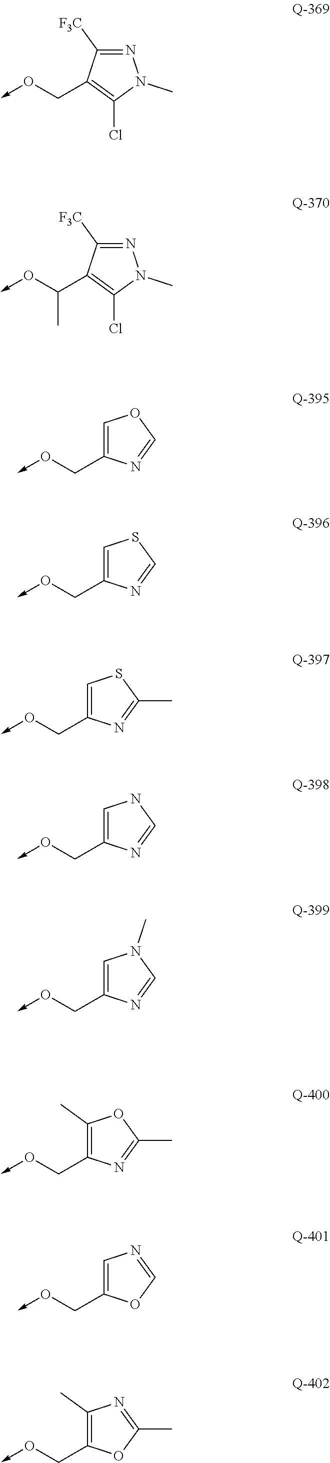

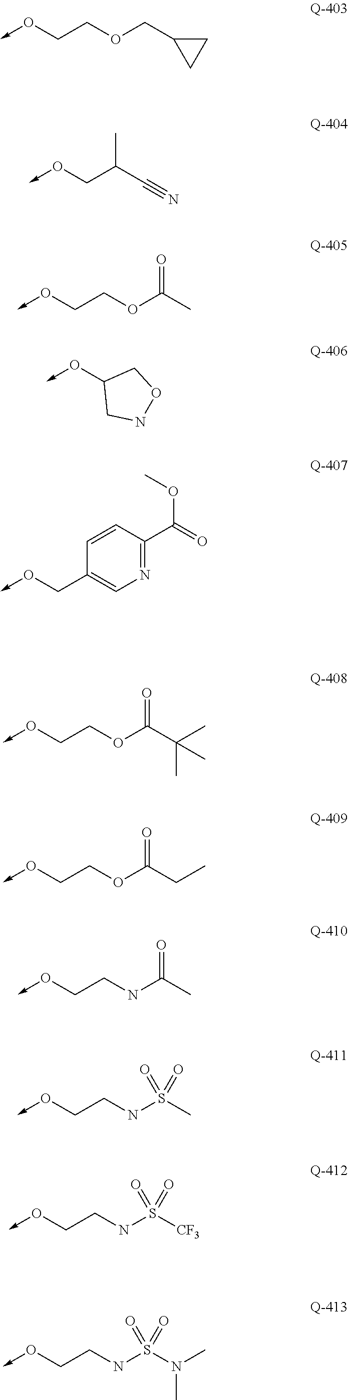

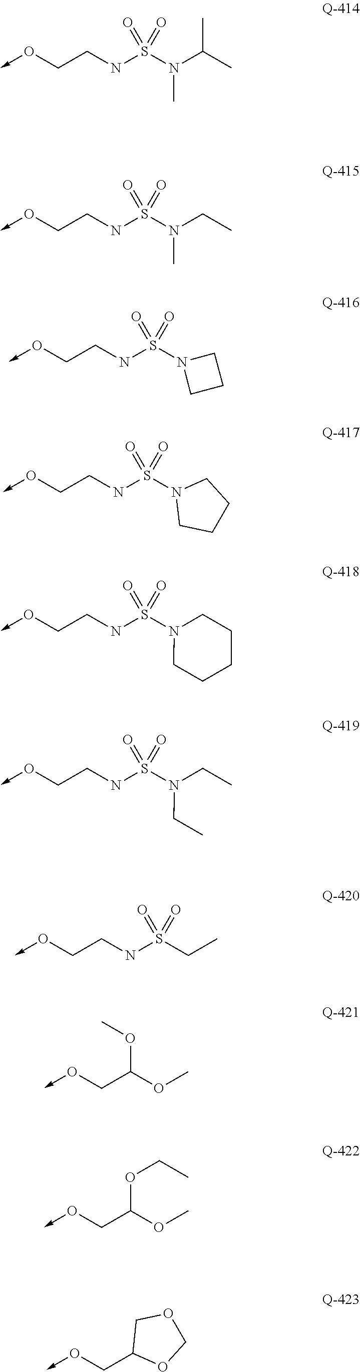

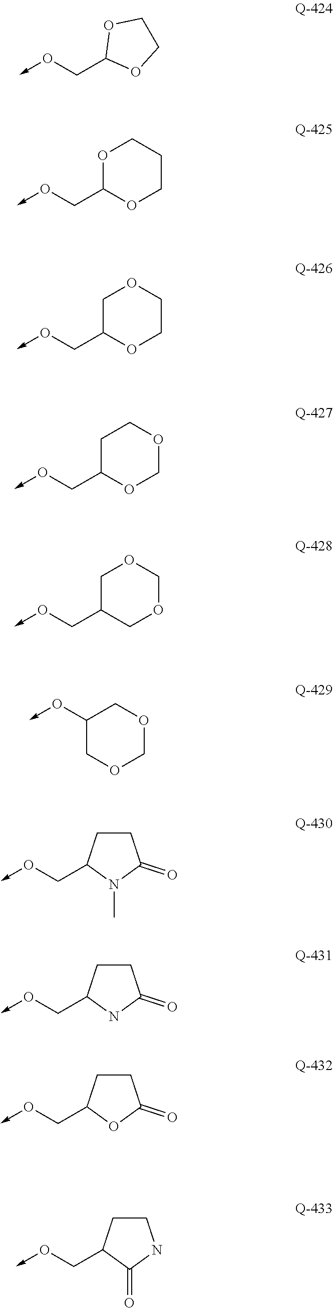

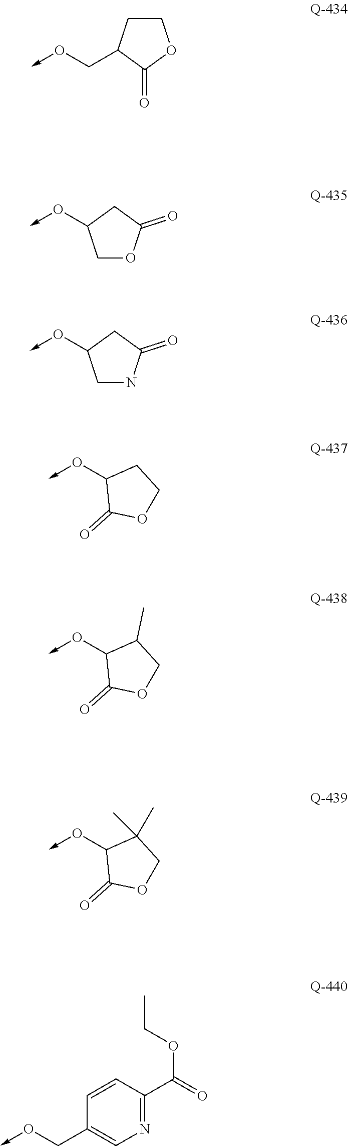

Examples of such herbicidally active compounds within the scope of formula (I) include:

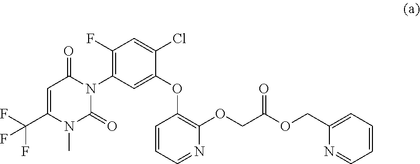 [SMILES: Cn1c(C(F)(F)F)cc(=O)n(-c2cc(Oc3cccnc3OCC(=O)OCc3ccccn3)c(Cl)cc2F)c1=O]

pyridin-2-ylmethyl 1(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate;

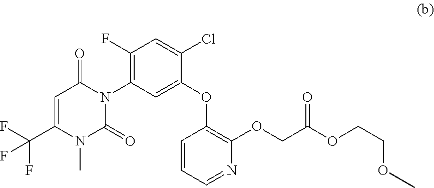

2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate;

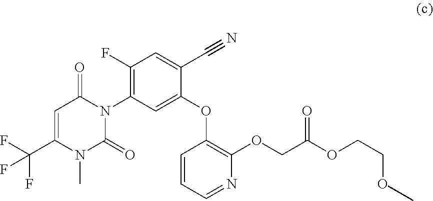

2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate; and

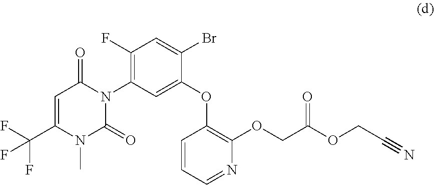 [SMILES: Cn1c(C(F)(F)F)cc(=O)n(-c2cc(Oc3cccnc3OCC(=O)OCC#N)c(Br)cc2F)c1=O]

cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate.

2) an herbicidally active compound of the general formula (II) or an agrochemically acceptable salt thereof

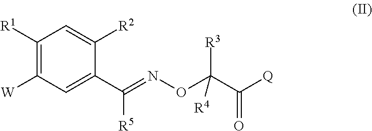

in which:
W represents a group W-1 to W-3

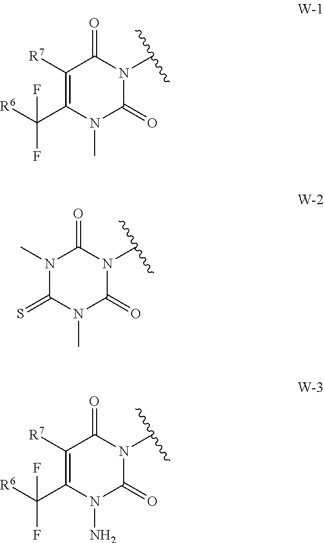

R¹ represents hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents fluorine, chlorine, bromine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl, R¹³O-(C₁-C₈)-alkyl, (C₃-C₈)-cycloalkyl, (C₂-C₈)-alkenyl, aryl-(C₁-C₈)-alkyl, heteroaryl-(C₁-C₈)-alkyl, or heterocyclyl-(C₁-C₈)-alkyl,
or
R³ and R⁴ together with the carbon atom to which they are bonded form a fully saturated or partly saturated 3- to 10-membered carbocyclic ring optionally having further substitution,
R⁵ represents hydrogen, (C₁-C₈)-alkyl, (C₁-C₈)-haloalkyl, R¹³O-(C₁-C₈)-alkyl, (C₂-C₈)-alkenyl, aryl-(C₁-C₈)-alkyl, heteroaryl-(C₁-C₈)-alkyl, heterocyclyl-(C₁-C₈)-alkyl, (C₃-C₈)-cycloalkyl, aryl, heteroaryl, or heterocyclyl,
R⁶ represents hydrogen, fluorine, chlorine, bromine, trifluoromethyl, difluoromethyl, methoxy, ethoxy, prop-1-yloxy, or but-1-yloxy,
R⁷ represents hydrogen or methyl,
Q represents hydroxy or a group Q-1, Q-2

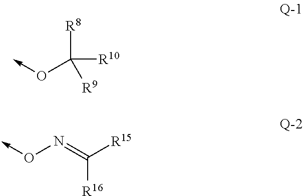

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl,
R⁹ represents hydrogen or (C₁-C₈)-alkyl,
R¹⁰ represents hydrogen, halogen, cyano, nitro, (C₁C₈)-alkyl, (C₁C₈)-haloalkyl, (C₃C₈)-cycloalkyl, (C₃C₈)-cycloalkyl-(C₁C₈)-alkyl, (C₃C₈)-halocycloalkyl, (C₃C₈)-halocycloalkyl-(C₁C₈)-alkyl, (C₂C₈)-alkenyl, (C₂C₈)-alkynyl, aryl, aryl-(C₁C₈)-alkyl, heteroaryl, heteroaryl-(C₁C₈)-alkyl, heterocyclyl, heterocyclyl-(C₁C₈)-alkyl, R¹¹R¹²N—(C₁C₈)-alkyl, R¹³O-(C₁C₈)-alkyl, cyano-(C₁C₈)-alkyl, (C₁C₈)-alkylcarbonyloxy-(C₁C₈)-alkyl, (C₃C₈)-cycloalkylcarbonyloxy-(C₁C₈)-alkyl, arylcarbonyloxy-(C₁C₈)-alkyl, heteroarylcarbonyloxy-(C₁C₈)-alkyl, heterocyclylcarbonyloxy-(C₁C₈)-alkyl, OR¹³, NR¹¹R¹², SR¹⁴, S(O)R¹⁴, SO₂R¹⁴, R¹⁴S—(C₁C₈)-alkyl, R¹⁴(O)S—(C₁C₈)-alkyl, R¹⁴O₂S—(C₁C₈)-alkyl, tris-[(C₁C₈)-alkyl]silyl-(C₁C₈)-alkyl, bis-[(C₁C₈)-alkyl](aryl)silyl(C₁C₈)-alkyl, [(C₁C₈)-alkyl]-bis-(aryl)silyl-(C₁C₈)-alkyl, tris-[(C₁C₈)-alkyl]silyl, bis-hydroxyboryl-(C₁C₈)-alkyl, bis-[(C₁C₈)-alkoxy]boryl-(C₁C₈)-alkyl, tetramethyl-1,3,2-Dioxaborolan-2-yl, tetramethyl-1,3,2-dioxaborolan-2-yl-(C₁C₈)-alkyl, nitro-(C₁C₈)-alkyl, C(O)OR¹³, C(O)R¹³, C(O)NR¹¹R¹², R¹³O(O)C—(C₁C₈)-alkyl, R¹¹R¹²N(O)C—(C₁C₈)-alkyl, or bis-(C₁C₈)-alkoxy-(C₁C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₅)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl, (C₁-C₈)-alkylcarbonyloxy-(C₁-C₈)-alkyl, (C₃-C₅)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₅)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₅)-cycloalkyl-[(C₁-C₈)-alkyl]amino, N-azetidinyl, N-pyrrolidinyl, N-piperidinyl, or N-morpholinyl, and
R¹⁵ and R¹⁶ independently of each other represent (C₁-C₈)-alkyl, (C₃-C₅)-cycloalkyl, aryl, heteroaryl, or heterocyclyl.

Examples of such herbicidally active compounds within the scope of formula (II) include methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-({2-chloro-4-fluoro-5-[3-methyl-2,6-dioxo-4-(trifluoromethyl)-3,6-dihydropyrimidin-1(2H)-yl]phenylImethylidene)amino]oxy}propanoate (also known as methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} propanoate or flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy Ipropanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy} propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, and (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid.

3) an herbicidally active compound of the general formula (III) or an agrochemically acceptable salt thereof

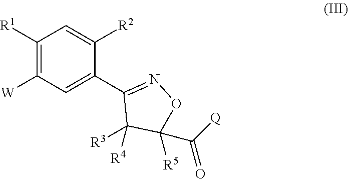

in which:
W represents a group W-1 to W-3

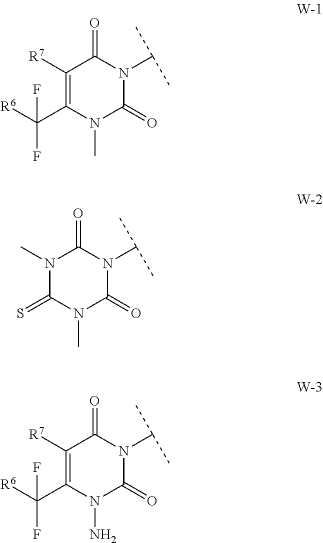

R¹ represents hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents fluorine, chlorine, bromine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl,
R⁵ represents hydrogen, (C₁-C₈)-alkyl, (C₁-C₈)-haloalkyl, R¹³O-(C₁-C₈)-alkyl, (C₂-C₈)-alkenyl, aryl-(C₁-C₈)-alkyl, heteroaryl-(C₁-C₈)-alkyl, or heterocyclyl-(C₁-C₈)-alkyl, or
R³ and R⁵ together with the carbon atom to which they are bonded form a fully saturated or partly saturated 3- to 10-membered monocyclic or bicyclic ring optionally interrupted by heteroatoms and optionally having further substitution,
R⁶ represents hydrogen, fluorine, chlorine, bromine, trifluoromethyl, difluoromethyl, methoxy, ethoxy, prop-1-yloxy, or but-1-yloxy,
R⁷ represents hydrogen, methyl,
Q represents hydroxy or a group Q-1, Q-2

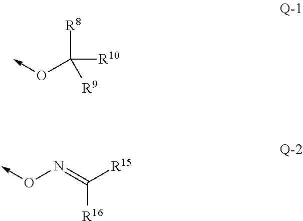

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl,
R⁹ represents hydrogen, (C₁-C₈)-alkyl,
R¹⁰ represents hydrogen, halogen, cyano, nitro, (C₁-C₈)-alkyl, (C₁-C₈)-aaloalkyl, (C₃-C₈)-cycloalkyl, (C₃-C₈)-cycloalkyl-(C₁-C₈)-alkyl, (C₃-C₈)-halocycloalkyl, (C₃-C₅)-halocycloalkyl-(C₁-C₈)-alkyl, (C₂-C₈)-alkenyl, (C₂-C₈)-alkynyl, aryl, aryl-(C₁-C₈)-alkyl, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O-(C₁-C₈)-alkyl, cyano-(C₁-C₈)-alkyl, (C₁-C₈)-alkylcarbonyloxy-(C₁-C₈)-alkyl, (C₃-C₅)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁—C₈)-alkyl, bis-[(C₁-C₈)-alkoxy]boryl-(C₁-C₈)-alkyl, tetramethyl-1,3,2-Dioxaborolan-2-yl, tetramethyl-1,3,2-dioxaborolan-2-yl-(C₁-C₈)-alkyl, nitro-(C₁-C₈)-alkyl, C(O)OR¹³, C(O)R¹³, C(O)NR¹¹R¹², R¹³O(O)C—(C₁-C₈)-alkyl, R¹¹R¹²N(O)C—(C₁-C₈)-alkyl, or bis-(C₁-C₈)-alkoxy-(C₁-C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁—C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl, (C₁-C₈)-alkylcarbonyloxy-(C₁-C₈)-alkyl, (C₃-C₅)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₅)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₅)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₅)-cycloalkyl-[(C₁-C₈)-alkyl]amino, N-azetidinyl, N-pyrrolidinyl, N-piperidinyl, or N-morpholinyl, and
R¹⁵ and R¹⁶ independently of each other represent (C₁-C₈)-alkyl, (C₃-C₅)-cycloalkyl, aryl, heteroaryl, or heterocyclyl.

Examples of the herbicidally active compounds within the scope of formula (III) include ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl1-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, and methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate.

4) an herbicidally active compound corresponding to a compound selected from the group consisting of A1, A2, A3, A4, A5, A6, A7, A8 and A9, or an agrochemically acceptable salt thereof, wherein:
A1 corresponds to:

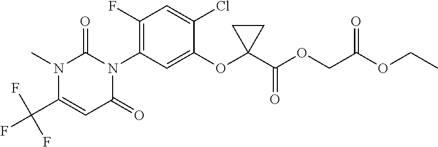 [SMILES: CCOC(=O)COC(=O)C1(Oc2cc(-n3c(=O)cc(C(F)(F)F)n(C)c3=O)c(F)cc2Cl)CC1]

2-ethoxy-2-oxoethyl 1-{2-chloro-4-fluoro-5-1[3-methyl-2,6-dioxo-4-(trifluoromethyl)-3,6-dihydropyrimidin-1(2H)-yl]phenoxy}cyclopropanecarboxylate;
A2 corresponds to:

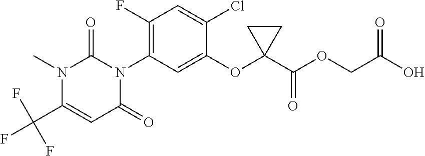 [SMILES: Cn1c(C(F)(F)F)cc(=O)n(-c2cc(OC3(C(=O)OCC(=O)O)CC3)c(Cl)cc2F)c1=O]

{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid;
A3 corresponds to:

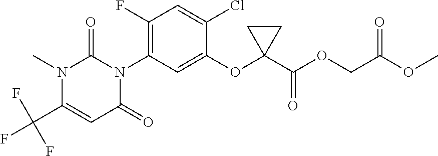 [SMILES: COC(=O)COC(=O)C1(Oc2cc(-n3c(=O)cc(C(F)(F)F)n(C)c3=O)c(F)cc2Cl)CC1]

2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
A4 corresponds to:

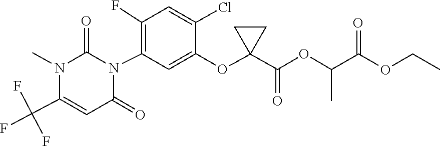 [SMILES: CCOC(=O)C(C)OC(=O)C1(Oc2cc(-n3c(=O)cc(C(F)(F)F)n(C)c3=O)c(F)cc2Cl)CC1]

1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
A5 corresponds to:

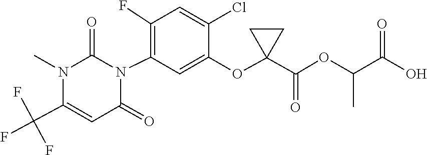 [SMILES: CC(OC(=O)C1(Oc2cc(-n3c(=O)cc(C(F)(F)F)n(C)c3=O)c(F)cc2Cl)CC1)C(=O)O]

2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid;
A6 corresponds to:

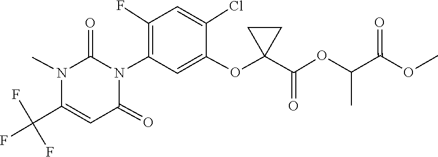 [SMILES: COC(=O)C(C)OC(=O)C1(Oc2cc(-n3c(=O)cc(C(F)(F)F)n(C)c3=O)c(F)cc2Cl)CC1]

1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
A7 corresponds to:

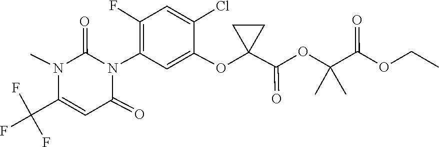 [SMILES: CCOC(=O)C(C)(C)OC(=O)C1(Oc2cc(-n3c(=O)cc(C(F)(F)F)n(C)c3=O)c(F)cc2Cl)CC1]

1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
A8 corresponds to:

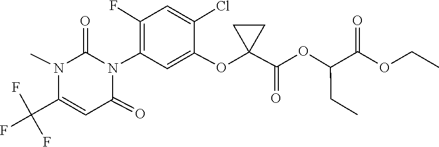 [SMILES: CCOC(=O)C(CC)OC(=O)C1(Oc2cc(-n3c(=O)cc(C(F)(F)F)n(C)c3=O)c(F)cc2Cl)CC1]

1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate; and
A9 corresponds to:

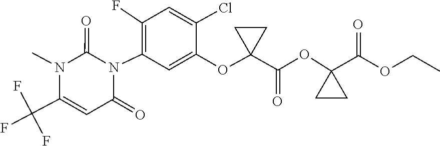 [SMILES: CCOC(=O)C1(OC(=O)C2(Oc3cc(-n4c(=O)cc(C(F)(F)F)n(C)c4=O)c(F)cc3Cl)CC2)CC1]

1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate.

5) an herbicidally active compound of the general formula (IV) or an agrochemically acceptable salt thereof

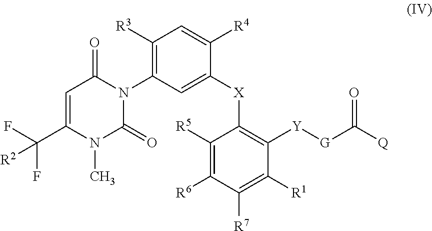

wherein
R¹ is hydrogen, fluoro, chloro, bromo, iodo, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluormethyl, difluormethyl, pentafluorethyl, 2,2-difluorethyl, 2,2,2-trifluorethyl, methoxy, ethoxy, prop-1-yloxy, prop-2-yloxy, but-1-yloxy, but-2-yloxy, 2-methylprop-1-yloxy, 1,1-dimethyleth-1-yloxy, difluormethoxy, trifluormethoxy, pentafluorethoxy, 2,2-difluorethoxy, or 2,2,2-trifluorethoxy,
R² is hydrogen, fluoro, chloro, bromo, methyl, trifluormethyl, methoxy, ethoxy, prop-1-yloxy, or but-1-yloxy,
R³ is hydrogen, fluoro, chloro, bromo, methoxy, ethoxy, prop-1-yloxy, prop-2-yloxy, but-1-yloxy, but-2-yloxy, 2-methylprop-1-yloxy, or 1,1-dimethyleth-1-yloxy,
R⁴ is fluoro, chloro, bromo, cyano, NO₂, C(O)NH₂, C(S)NH₂, trifluormethyl, difluormethyl, pentafluorethyl, ethinyl, propin-1-yl, 1-butin-1-yl, pentin-1-yl, or hexin-1-yl,
R⁵, R⁶ and R⁷ are independently from each other hydrogen, fluoro, chloro, bromo, iodo, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluormethyl, difluormethyl, pentafluorethyl, 2,2-difluorethyl, 2,2,2-trifluorethyl, methoxy, ethoxy, prop-1-yloxy, prop-2-yloxy, but-1-yloxy, but-2-yloxy, 2-methylprop-1-yloxy, 1,1-dimethyleth-1-yloxy, difluormethoxy, trifluormethoxy, pentafluorethoxy, 2,2-difluorethoxy, or 2,2,2-trifluorethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylen, (1-methyl)ethyl-1-en, (2-methyl)ethyl-1-en, n-butylen, 1-methylpropyl-1-en, 2-methylpropyl-1-en, 3-methylpropyl-1-en, 1,1-dimethylethyl-1-en, 2,2-dimethylethyl-1-en, 1-ethylethyl-1-en, 2-ethylethyl-1-en, 1-(prop-1-yl)ethyl-1-en, 2-(prop-1-yl)ethyl-1-en, 1-(prop-2-yl)ethyl-1-en, 2-(prop-2-yl)ethyl-1-en, 1,1,2-trimethylethyl-1-en, 1,2,2-trimethylethyl-1-en, 1,1,2,2-tetramethylethyl-1-en, n-pentylen, 1-methylbutyl-1-en, 2-methylbutyl-1-en, 3-methylbutyl-1-en, 4-methylbutyl-1-en, 1,1-dimethylpropyl-1-en, 2,2-dimethylpropyl-1-en, 3,3-dimethylpropyl-1-en, 1,2-dimethylpropyl-1-en, 1,3-dimethylpropyl-1-en, 1-ethylpropyl-1-en, n-hexylen, 1-methylpentyl-1-en, 2-methylpentyl-1-en, 3-methylpentyl-1-en, 4-methylpentyl-1-en, 1,1-dimethylbutyl-1-en, 1,2-dimethylbutyl-1-en, 1,3-di-methylbutyl-1-en, 2,2-dimethylbutyl-1-en, 2,3-dimethylbutyl-1-en, 3,3-dimethylbutyl-1-en, 1-ethylbutyl-1-en, 2-ethylbutyl-1-en, 1,1,2-trimethylpropyl-1-en, 1,2,2-trimethylpropyl-1-en, 1-ethyl-1-methylpropyl-1-en, or 1-ethyl-2-methylpropyl-1-en,
X and Y are independently from each other O (oxygen) or S (sulfur)
and
Q is one of the groups Q-1 to Q-25, wherein in the formulae of the following table the arrow stands for a bond of the respective group Q to the carbonyl group in the general formula (I):

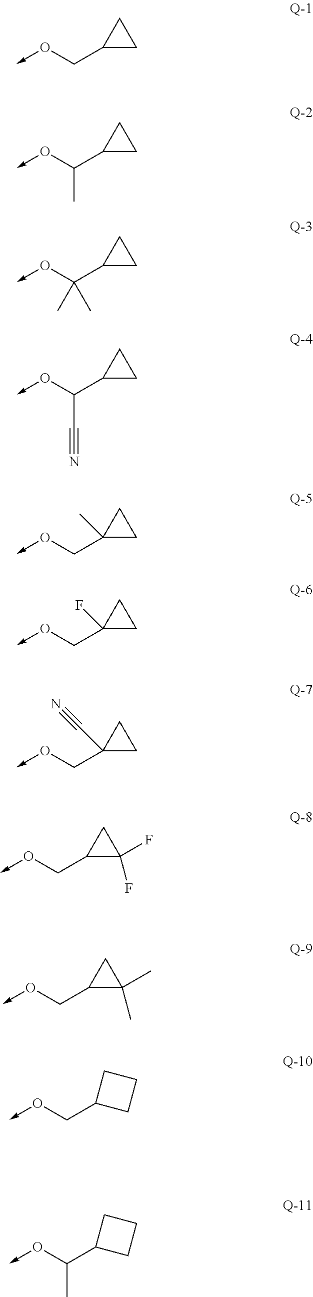

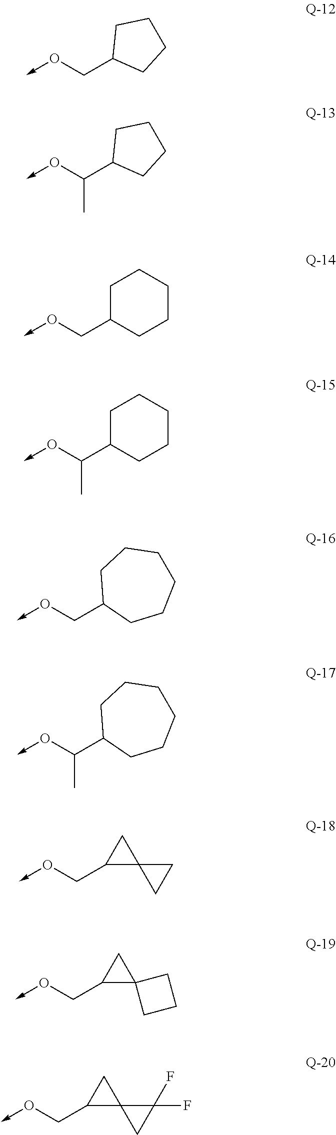

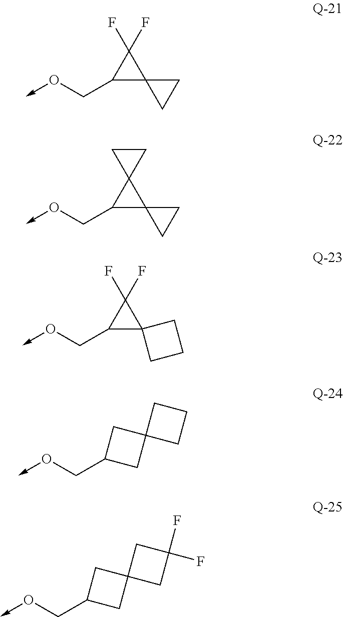

An example of an herbicidally active compound within the scope of formula (IV) is cyclopropylmethyl-(2-{2-chlor-4-fluor-5-[3-methyl-2,6-dioxo-4-(trifluormethyl)-3,6-dihydropyrimidin-1(2H)-yl]phenoxy}phenoxy)acetate, which has the following structure:

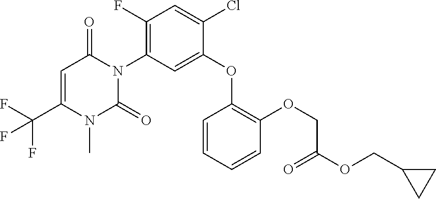 [SMILES: Cn1c(C(F)(F)F)cc(=O)n(-c2cc(Oc3ccccc3OCC(=O)OCC3CC3)c(Cl)cc2F)c1=O]

6) an herbicidally active compound of the general formula (V) or an agrochemically acceptable salt thereof

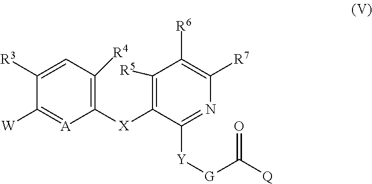

wherein
W represents a group W-1 to W-2

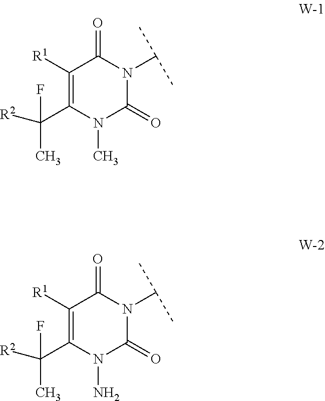

A represents nitrogen or CH;
R¹ represents hydrogen or methyl;
R² represents hydrogen or fluorine;
R³ represents hydrogen, halogen, or (C₁-C₈)-alkoxy;
R⁴ represents halogen, cyano, NO₂, C(O)NH₂, C(S)NH₂, (C₁-C₈)-haloalkyl, or (C₂-C₈)-alkynyl;
R⁵, R⁶, and R⁷ independently of one another represent hydrogen, halogen, cyano, (C₁-C₈)-alkyl, (C₁-C₈)-haloalkyl, (C₁-C₈)-alkoxy, or (C₁-C₈)-haloalkoxy;
G represents a branched or unbranched alkylene group optionally substituted with an (C₁-C₃)-alkoxy group;
Q represents hydroxy or a group Q-1, Q-2;

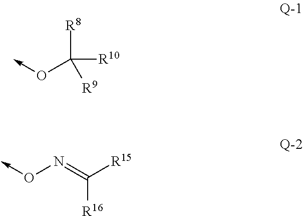

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl;
R⁹ represents hydrogen or (C₁-C₈)-alkyl;
R¹⁰ represents hydrogen, halogen, cyano, NO₂, (C₁-C₈)-alkyl, (C₁-C₈)-haloalkyl, (C₃-C₈)-cycloalkyl, (C₃-C₅)-cycloalkyl-(C₁-C₈)-alkyl, (C₃-C₅)-halocycloalkyl, (C₃-C₈)-halocycloalkyl-(C₁-C₈)-alkyl, (C₂-C₈)-alkenyl, (C₂-C₈)-alkynyl, aryl, aryl-(C₁-C₈)-alkyl, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O-(C₁-C₈)-alkyl, cyano-(C₁-C₈)-alkyl, (C₁-C₈)-alkylcarbonyloxy-(C₁-C₅)-alkyl, (C₃-C₅)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₅)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈)-alkyl, C(O)OR¹³, C(O)R¹³, C(O)NR¹¹R¹², R¹³O(O)C—(C₁-C₈)-alkyl, R¹¹R¹²N(O)C—(C₁-C₅)-alkyl, or bis-(C₁-C₈)-alkoxy-(C₁-C₈)-alkyl, or
R⁸ and R¹⁰ together with the carbon atom to which they are attached form a fully saturated or partially saturated 3- to 10-membered monocyclic or bicyclic ring optionally interrupted by heteroatoms and optionally having further substitution; R¹¹ and R¹² are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution;
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, (C₁-C₈)-alkylcarbonyloxy-(C₁-C₈)-alkyl, (C₃-C₅)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl;
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkyl-amino, aryl-(C₁-C₈)-amino, aryl-(C₁-C₆)-alkyl-amino, aryl-[(C₁-C₈)-alkyl]amino, (C₃-C₅)-cycloalkyl-amino, (C₃-C₈)-cycloalkyl-[(C₁-C₈)-alkyl]amino; N-azetidinyl, N-pyrrolidinyl, N-piperidinyl, or N-morpholinyl;
R¹⁵ and R¹⁶ independently of each other represent (C₁-C₈)-alkyl, (C₃-C₅)-cycloalkyl, aryl, heteroaryl, or heterocyclyl or
R¹⁵ and R¹⁶ together with the nitrogen atom to which they are attached form an imino group which is optionally substituted further by hydrogen, (C₁-C₈)-alkyl, aryl-(C₁-C₈)-alkyl, (C₃-C₈)-cycloalkyl, aryl, heteroaryl, heterocyclyl, (C₁-C₈)-alkoxycarbonyl-(C₁-C₈)-alkyl, aryl-(C₁-C₈)-alkoxycarbonyl-(C₁-C₈)-alkyl;
X and Y, independently of one another, represent oxygen or sulfur; and
Z represents nitrogen or CH.

Examples of the herbicidally active compounds within the scope of formula (V) include ethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetic acid, ethyl (2-{2-chloro-5-[4-(1,1-difluoroethyl)-3-methyl-2,6-dioxo-3,6-dihydropyrimidin-1(2H)-yl]-4-fluorophenoxy}phenoxy)acetate, (2-{2-chloro-5-[4-(1,1-difluoroethyl)-3-methyl-2,6-dioxo-3,6-dihydropyrimidin-1(2H)-yl]-4-fluorophenoxy}phenoxy)acetic acid, ethyl (2-{2-chloro-4-fluoro-5-[4-(1-fluoroethyl)-3-methyl-2,6-dioxo-3,6-dihydropyrimidin-1(2H)-yl]phenoxy}phenoxy)acetate, 2-methoxyethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, tetrahydrofuran-2-ylmethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, cyanomethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, methyl (2-{2-chloro-5-[4-(1,1-difluoroethyl)-3-methyl-2,6-dioxo-3,6-dihydropyrimidin-1(2H)-yl]-4-fluorophenoxy}phenoxy)(methoxy)acetate, methyl (2-{2-bromo-5-[4-(1,1-difluoroethyl)-3-methyl-2,6-dioxo-3,6-dihydropyrimidin-1(2H)-yl]-4-fluorophenoxy}phenoxy)(methoxy)acetate, [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetic acid, ethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, 2-methoxyethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, tetrahydrofuran-2-ylmethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, and ethyl 2-[[3-[5-[4-(1,1-difluoroethyl)-3-methyl-2,6-dioxo-pyrimidin-1-yl]-4-fluoro-2-nitro-phenoxy]-2-pyridyl]oxy]acetate.

7) an herbicidally active compound corresponding to a compound selected from the group consisting of A1, A2, A3, A4, A5, A6, A7, A8, A9, A10, A1 l and A12, or an agrochemically acceptable salt thereof,
wherein
A1 corresponds to:

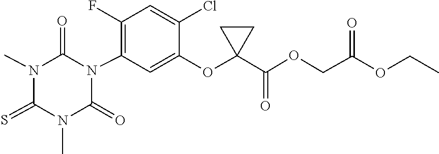 [SMILES: CCOC(=O)COC(=O)C1(Oc2cc(-n3c(=O)n(C)c(=S)n(C)c3=O)c(F)cc2Cl)CC1]

2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate;
A2 corresponds to:

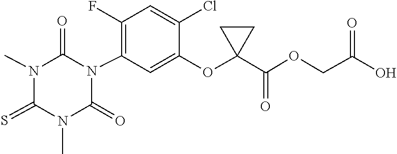 [SMILES: Cn1c(=S)n(C)c(=O)n(-c2cc(OC3(C(=O)OCC(=O)O)CC3)c(Cl)cc2F)c1=O]

[({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid;
A3 corresponds to:

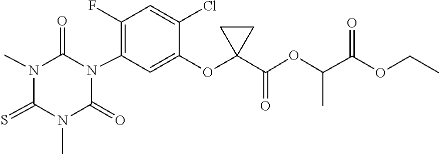 [SMILES: CCOC(=O)C(C)OC(=O)C1(Oc2cc(-n3c(=O)n(C)c(=S)n(C)c3=O)c(F)cc2Cl)CC1]

1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate;
A4 corresponds to:

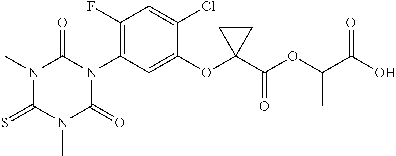 [SMILES: CC(OC(=O)C1(Oc2cc(-n3c(=O)n(C)c(=S)n(C)c3=O)c(F)cc2Cl)CC1)C(=O)O]

2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid;
A5 corresponds to:

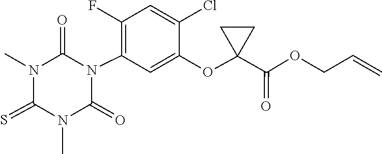 [SMILES: C=CCOC(=O)C1(Oc2cc(-n3c(=O)n(C)c(=S)n(C)c3=O)c(F)cc2Cl)CC1]

allyl 1-[2-chloro-5-(3,5-dimethyl-2,6-dioxo-4-sulfanylidene-1,3,5-triazinan-1-yl)-4-fluorophenoxy]cyclopropanecarboxylate;
A6 corresponds to:

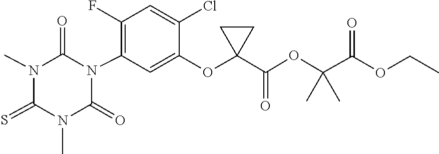 [SMILES: CCOC(=O)C(C)(C)OC(=O)C1(Oc2cc(-n3c(=O)n(C)c(=S)n(C)c3=O)c(F)cc2Cl)CC1]

1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate;
A7 corresponds to:

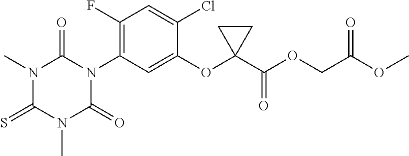

2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate;
A8 corresponds to:

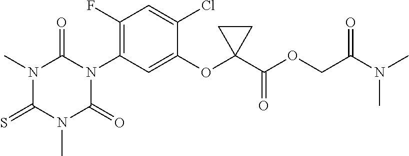

2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate;
A9 corresponds to:

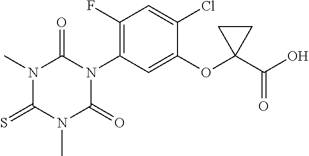 [SMILES: Cn1c(=S)n(C)c(=O)n(-c2cc(OC3(C(=O)O)CC3)c(Cl)cc2F)c1=O]

1-[2-chloro-5-(3,5-dimethyl-2,6-dioxo-4-sulfanylidene-1,3,5-triazinan-1-yl)-4-fluorophenoxy]cyclopropanecarboxylic acid;
A10 corresponds to:

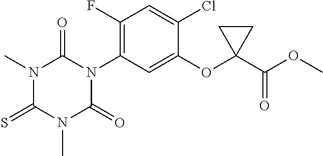

methyl 1-[2-chloro-5-(3,5-dimethyl-2,6-dioxo-4-sulfanylidene-1,3,5-triazinan-1-yl)-4-fluorophenoxy]cyclopropanecarboxylate;
A11 corresponds to:

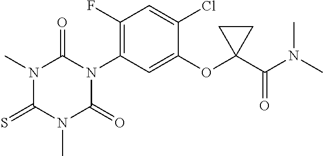

1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide; and
A12 corresponds to:

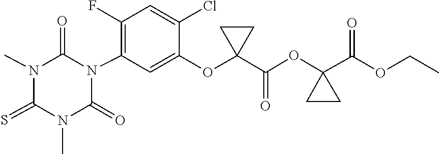 [SMILES: CCOC(=O)C1(OC(=O)C2(Oc3cc(-n4c(=O)n(C)c(=S)n(C)c4=O)c(F)cc3Cl)CC2)CC1]

ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate.

As used herein, benzoic acid auxins or herbicides include, but are not limited to, dicamba (3,6-dichloro-2-methoxybenzoic acid), dicamba salts, dicamba-butotyl, dicamba-diglycolamine salt, dicamba-dimethylammonium, dicamba-diethanolammonium, dicamba-isopropylammonium, dicamba-potassium, dicamba-sodium, and dicamba-trolamine.

As used herein, inhibitors of glutamine synthetase include, but are not limited to, phosphinothricin, glufosinate, glufosinate salts, glufosinate-ammonium, glufosinate-sodium, glufosinate-P, L-glufosinate-ammonium, L-glufosinate-sodium, and combinations of any thereof.

As used herein, the ß-triketone HPPD inhibitor can be selected from the group consisting of mesotrione, benzobicyclon (BBC), tembotrione, sulcotrione, tefuryltrione, and combinations of any thereof.

As used herein, inhibitors of 5-enolpyruvylshikimate-3-phosphate synthase (EPSPS) include, but are not limited to, glyphosate, glyphosate salts, glyphosate-isopropylammonium, glyphosate-ammonium, glyphosate-dimethylammonium, glyphosate-trimesium (=sulfosate), glyphosate-diammonium, glyphosate-potassium, glyphosate-sodium, and combinations of any thereof.

As used herein, “herbicide tolerant” or “herbicide tolerance” or “tolerance” means the ability to be wholly or partially unaffected by the presence or application of one of more herbicide(s), for example to resist the toxic effects of an herbicide when applied. A cell, seed, or plant is “herbicide tolerant” or has “improved tolerance” if it can maintain at least some normal growth or phenotype in the presence of one or more herbicide(s). A trait is an herbicide tolerance trait if its presence can confer improved tolerance to an herbicide upon a cell, plant, or seed as compared to the wildtype or control cell, plant, or seed. Crops comprising an herbicide tolerance trait can continue to grow in the presence of the herbicide and may be minimally affected by the presence of the herbicide. A protein confers “herbicide tolerance” if expression of the protein can confer improved tolerance to an herbicide upon a cell, plant, or seed as compared to the wildtype or control cell, plant, or seed. Examples of herbicide tolerance proteins are protoporphyrinogen oxidase, dicamba monooxygenase, phosphinothricin N-acetyltransferase, the alpha-ketoglutarate-dependent non-heme iron dioxygenase, triketone dioxygenase, and 5-enolpyruvylshikimate-3-phosphate synthase. Herbicide tolerance may be complete or partial insensitivity to a particular herbicide and may be expressed as a percent (%) tolerance or insensitivity to a particular herbicide.

As used herein “herbicide injury” or “injury” refers to injury to a plant because of the application of one or more herbicides. The “injury rate” or “percent injury” refers to the percentage of leaf area of a plant exhibiting damage such as necrosis (brown or dead tissue), chlorosis (yellow tissue or yellow spotting) and malformation (misshapen leaves or plant structures, epinasty or twisting of stem, cupping of leaves) caused by herbicide application based on visual evaluation. It is measured on a scale of 0 to 100, where “0” representing no crop injury and “100” denoting complete crop injury (death).

For cotton plants containing or comprising cotton event Gh_CSM63718, the plant will have decreased injury after application of one or more herbicides selected from the group consisting of glufosinate, ß-triketone HPPD inhibitors, dicamba, glyphosate, PPO inhibitors, and combinations of any thereof. For example, cotton plants containing or comprising cotton event Gh_CSM63718 will have less than about 5% injury, less than about 10% injury, less than about 15% injury, or less than about 20% injury following application of glufosinate, a ß-triketone HPPD inhibitor such as mesotrione, dicamba, glyphosate, and/or a PPO herbicide such as flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate, and combinations of any thereof, as compared to otherwise identical cotton plants that do not contain cotton event Gh_CSM63718.

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant). Weeds are commonly known in the art and vary by geography, season, growing environment, and time. Lists of weed species are available from agricultural and scientific societies and efforts (such as the Weed Science Society of America, the Canadian Weed Science Society, the Brazilian Weed Science Society, the International Weed Science Society, and the International Survey of Herbicide Resistant Weeds), government agencies (such as the United States Department of Agriculture and the Australia Department of the Environment and Energy), and industry and farmer associations. Major troublesome weeds in cotton production include waterhemp (Amaranthus tuberculatus), ragweed (Ambrosia spp.) such as giant ragweed (Ambrosia trifida), and common ragweed (Ambrosia artemissifolia), common lambsquarters (Chenopodium album), morning glory species (Ipomoea spp.), horseweed (Conyza canadensis), marestail (Erigeron canadensis), palmer amaranth (Amaranthus palmeri), pigweed (Amaranthus spp.), velvetleaf (Abutilon theophrasti Medik.), common cocklebur (Xanthium strumarium), foxtail (Setaria spp.), crabgrass (Digitaria spp.), barnyard grass (Echinochloa crus-galli), Johnsongrass (Sorghum halepense), and thistles (Cirsium spp.) (Heap 2021; Shoup et al, 2016).

Methods for controlling or preventing weed growth in an area are provided. One example of such a method comprises growing cotton comprising event Gh_CSM63718 in the area and applying an effective amount of at least one herbicide selected from the group consisting of glufosinate, a ß-triketone HPPD inhibitor herbicide, dicamba, glyphosate, a PPO herbicide, and any combination thereof to control weeds in the area without injury to the cotton or with less than about 10% injury to the cotton. The methods comprise applying one or more of the foregoing herbicides, where seeds or plants comprising cotton event Gh_CSM63718 are planted in the area before, at the time of, or after applying the one or more herbicides and the herbicide application prevents or inhibits weed growth and does not injure the cotton plants comprising event Gh_CSM63718 or has about less than about 5-20% injury. The plant growth area may or may not comprise weed seeds or plants at the time of herbicide application. The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after cotton seed planting), or both. For example, a method for controlling weeds is provided that consists of planting seed comprising cotton event Gh_CSM63718 in an area and applying an herbicidally effective amount over the growing season of one or more herbicide selected from the group consisting of glufosinate, a ß-triketone HPPD inhibitor herbicide, dicamba, glyphosate, a PPO herbicide alone or in any combination thereof, for the purpose of controlling weeds in the area with no injury or less than about 5-20% injury to the plants containing cotton event Gh_CSM63718. Such application of herbicide(s) may be pre-planting (any time prior to planting seed comprising cotton event Gh_CSM63718, including for burn-down purposes, that is application to emerging or existing weeds prior to seed plant), pre-emergence (any time after seed comprising cotton event Gh_CSM63718 is planted and before plants comprising cotton event Gh_CSM63718 emerge), or post-emergence (any time after plants comprising cotton event Gh_CSM63718 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Herbicide application in practicing the methods described herein may be at the recommended commercial rate or any fraction or multiple thereof, such as twice the recommended commercial rate. Herbicide rates may be expressed as pounds acid equivalent per acre (lb ae/acre), pounds active ingredient per acre (lb ai/acre) or pounds active ingredient per hectare (lb ai/ha), depending on the herbicide and the formulation. One gram per hectare is equal to 0.000892179 pound per acre. The use of acres or hectare in the herbicide application rates as provided herein is merely instructive; herbicide application rates in the equivalent dosages to any rate provided herein may be used for areas larger or smaller than an acre. The herbicide application can comprise at least one herbicide selected from the group consisting of glufosinate, a ß-triketone HPPD inhibitor herbicide, dicamba, glyphosate, a PPO herbicide, and any combination thereof. The ß-triketone HPPD inhibitor herbicide can be selected from the group consisting of mesotrione, benzobicyclon (BBC), tembotrione, sulcotrione, tefuryltrione, and combinations of any thereof. The PPO herbicide can include, but not limited to flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, fomesafen, saflufenacil, sulfentrazone, tiafenacil, and trifludimoxazin, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate, and combinations of any thereof. The plant growth area may or may not comprise weed plants at the time of herbicide application.

The effective amount of glufosinate can be about 0.4 lb/acre to about 1.6 lb/acre over a growing season. When the ß-triketone HPPD inhibitor comprises mesotrione, the effective amount of mesotrione can be about 0.09 lb/acre to about 0.36 lb/acre over a growing season. The effective amount of dicamba can be about 0.5 lb/acre to about 2 lb/acre over a growing season. The effective amount of glyphosate can be about 0.5 lb/acre to about 2.5 lb/acre over a growing season. The effective amount of the epyrifenacil PPO herbicide can be about 0.0009 lb/acre to about 1.5 lb/acre over a growing season. Table 3 provides examples of various PPO herbicides and the application rates that can be used for controlling weeds in a cotton crop growing area where Gh_CSM63718 is planted.

TABLE 3
Examples of PPO Herbicides and Application Rates

| | Broad | Intermediate | Labeled |
|---|---|---|---|
| | Rate Range | Rate Range | Rates |
| | (gram/ | (gram/ | (gram/ |
| PPO Herbicide | hectare) | hectare) | hectare) |
| Epyrifencacil (S3100) | 1-200 | 5-100 | 20-40 |
| Flumioxazin | 1-448 | 5-140 | 70-224 |
| Fomesafen | 1-1680 | 5-840 | 280-420 |
| Lactofen | 1-880 | 5-440 | 140-220 |
| Saflufenacil | 1-400 | 5-200 | 22-88 |
| Sulfentrazone | 1-1680 | 5-840 | 112-420 |
| Trifludimoxazin | 1-75 | 10-50 | 25-50 |
| Tiafenacil | 1-75 | 10-50 | 25-50 |
| Acifluorfen | 1-1680 | 5-840 | 280-560 |
| Pyraflufen | 1-50 | 10-25 | 25-50 |
| Oxadiazon | 1-400 | 50-200 | 200-400 |
| Butafenacil | 1-100 | 10-100 | 25-50 |
| pyridin-2-ylmethyl [(3-{2- | 1-200 | 10-100 | 50-100 |
| chloro-4-fluoro-5-[3- | | | |
| methyl-2,6-dioxo-4- | | | |
| (trifluoromethyl)-3,6- | | | |
| dihydropyrimidin-1(2H)- | | | |
| yl]phenoxy}pyridin-2- | | | |
| yl)oxy]acetate | | | |
| Flufenoximacil | 1-200 | 5-75 | 25-50 |
| 2-methoxyethyl [(3-{2- | 1-200 | 5-75 | 25-50 |
| chloro-4-fluoro-5-[3- | | | |
| methyl-2,6-dioxo-4- | | | |
| (trifluoromethyl)-3,6- | | | |
| dihydropyrimidin-1(2H)- | | | |
| yl]phenoxy}pyridin-2- | | | |
| yl)oxy]acetate | | | |
| cyanomethyl [(3-{2-bromo- | 1-200 | 5-75 | 25-50 |
| 4-fluoro-5-[3-methyl-2,6- | | | |
| dioxo-4-(trifluoromethyl)- | | | |
| 3,6-dihydropyrimidin-1(2H)- | | | |
| yl]phenoxy}pyridin-2- | | | |
| yl)oxy]acetate | | | |
| cyclopropylmethyl (2-{2- | 1-200 | 5-100 | 50-100 |
| chloro-4-fluoro-5-[3- | | | |
| methyl-2,6-dioxo-4- | | | |
| (trifluoromethyl)-3,6- | | | |
| dihydropyrimidin-1(2H)- | | | |
| yl]phenoxy}phenoxy)acetate; | | | |

Methods for controlling volunteer cotton comprising cotton event Gh_CSM63718 in an area are provided. The methods comprise applying an herbicidally effective amount of at least one herbicide other than glufosinate, a ß-triketone HPPD inhibitor herbicide, dicamba, glyphosate, or a PPO herbicide, wherein the herbicide application prevents growth of cotton comprising event Gh_CSM63718. Illustrative examples of such herbicides are pyrithiobac, fluometuron, trifloxysulfuron, paraquat, diquat, clopyralid, topramezone, atrazine, (3-(3,4-dichlorophenyl)-1,1-dimethylurea) (DCMU, sold under the tradename Diuron), 2,4-D, thidiazuron (TDZ), dichlorprop-p 2-ethylhexyl ester (sold under the tradename Duplosan), dichlorprop-p, and combinations of any thereof, wherein the herbicide application prevents growth of volunteer cotton comprising event Gh_CSM63718. For example, to control volunteer cotton comprising event Gh_CSM63718 in a cotton cultivation field, fluometuron and DCMU can be applied preemergence, and trifloxysulfuron and pyrithiobac can be applied postemergence. To control volunteer cotton comprising event Gh_CSM63718 in a corn cultivation field, atrazine can be applied preemergence and post emergence, and topramazone and clopyralid can be applied postemergence.

Methods for producing plants and seeds comprising cotton event Gh_CSM63718 are provided. Plants may be bred using any method known in the art. A progeny cotton plant comprising the event Gh_CSM63718 may be produced, for example, by selfing a parent plant or line comprising the event Gh_CSM63718, wherein such parent plant or line is homozygous or hemizygous for the event Gh_CSM63718, or by crossing a first parent plant or line comprising the event Gh_CSM63718, wherein such parent plant or line is homozygous or hemizygous for the event Gh_CSM63718, with a second parent plant or line having a different genotype or germplasm than the first parent line, wherein the second parent plant or line may or may not contain or comprise the event Gh_CSM63718. As described further herein, cotton event Gh_CSM63718 comprises five expression cassettes with the PAT cassette encoding phosphinothricin N-acetyltransferase (PAT), the TDO cassette encoding triketone dioxygenase, the DMO casstte encoding dicamba monooxygenase, the EPSPS cassette encoding 5-enolpyruvylshikimate-3-phosphate synthase, and the PPO cassette encoding protoporphyrinogen oxidase. According to some embodiments, the transgenic cotton plant(s) comprising the event Gh_CSM63718 is/are tolerant to glufosinate, ß-triketone HPPD inhibitor herbicides, dicamba, glyphosate, and PPO inhibitors, relative to a non-transgenic control plant. Transgenic cotton plants used in these methods may be homozygous or heterozygous for the transgene. Progeny plants produced by these methods may be varietal or hybrid plants; may be grown from seeds produced by cotton event Gh_CSM63718 containing plant and/or from seeds produced by a plant fertilized with pollen from a cotton event Gh_CSM63718 containing plant; and may be homozygous or heterozygous for the transgenes and/or event Gh_CSM63718. Progeny plants may be subsequently self-pollinated to generate a true breeding line of plants, i.e., plants homozygous for the transgene, or alternatively may be out-crossed, e.g., bred with another unrelated plant, to produce a varietal or a hybrid seed or plant.

A method of obtaining a seed of a cotton plant or a cotton plant that is tolerant to glufosinate, ß-triketone HPPD inhibitor herbicides, dicamba, glyphosate, and PPO herbicides, is provided. The method comprises: (a) obtaining a population of progeny seed or plants grown therefrom, at least one of which comprises cotton event Gh_CSM63718; and (b) identifying at least a first progeny seed or plant grown therefrom that comprises cotton event Gh_CSM63718. Identifying the progeny seed or plant grown therefrom that comprises cotton event Gh_CSM63718 can comprise: (a) growing the progeny seed or plant to produce progeny plants; (b) treating the progeny plants with an effective amount of glufosinate, a ß-triketone HPPD inhibitor herbicide, dicamba, glyphosate, a PPO herbicide, and combinations of any thereof; and (c) selecting a progeny plant that is tolerant to glufosinate, the ß-triketone HPPD inhibitor herbicide, dicamba, glyphosate, the PPO herbicide, and the combination(s) thereof. Alternatively or in addition, identifying the progeny seed or plant grown therefrom that comprises cotton event Gh_CSM63718 comprises detecting the presence of cotton event Gh_CSM63718 in a sample derived from the progeny seed or plant grown therefrom. Alternatively or in addition, identifying the progeny seed or plant grown therefrom that comprises cotton event Gh_CSM63718 comprises detecting the presence of the phosphinothricin N-acetyltransferase protein (PAT), the triketone dioxygenase protein (TDO), the dicamba monooxygenase protein (DMO), the 5-enolpyruvylshikimate-3-phosphate synthase protein (EPSPS), and the protoporphyrinogen oxidase (PPO) protein encoded by cotton event Gh_CSM63718 in a sample derived from the progeny seed or plant grown therefrom.

As used herein, the terms “line”, “breeding line”, “genotype” or “germplasm” are used interchangeably to refers to a group of plants that show little or no genetic variation between individuals for at least one trait. Such “line”, “breeding line”, “genotype” or “germplasm” can be created by self-pollination for several generations, selection, or vegetative propagation from a single parent using tissue or cell culture techniques. As used herein, the terms “cultivar” and “variety” are synonymous and refer to a line used for commercial production.

The production of double haploids may also be used to produce cotton plants and seeds homozygous for event Gh_CSM63718 DNA in a breeding program. Double haploids are produced by the doubling of a set of chromosomes (1 N) from a heterozygous plant to produce a completely homozygous individual. For example, see Wan, et al., (1989) and U.S. Pat. No. 7,135,615. This can be advantageous because the process omits the generations of selfing needed to obtain a homozygous plant from a heterozygous source. One way of producing haploid and double haploid cotton plant comprising event Gh_CSM63718 is through anther culture of flowers comprising event Gh_CSM63718 (Khan et al., 2010). Other methods such as natural polyembryony, induction with irradiated pollen, crosses with polyploid plants or wild species, unfertilized ovule and microspore culture, and DMP-based haploid induction system (Long et al., 2023) followed by chromosome doubling can also be applied to produce haploid and double haploid cotton plants comprising event Gh_CSM63718.

Seed and progeny plants made by the methods described herein comprise cotton event Gh_CSM63718. Application of one or more herbicide for which cotton event Gh_CSM63718 confers tolerance may be used to select progeny that comprise cotton event Gh_CSM63718. Alternatively, progeny may be analyzed using diagnostic methods to select for plants or seeds comprising cotton event Gh_CSM63718.

Cotton transgenic events are known to one of skill in the art; for example, a list of such traits is provided by the United States Department of Agriculture's (USDA) Animal and Plant Health Inspection Service (APHIS) and can be found on their website at www.aphis.usda.gov. Two or more transgenic events may thus be combined in a progeny seed or plant by crossing two parent plants each comprising one or more transgenic event(s), collecting progeny seed, and selecting for progeny seed or plants that contain the two or more transgenic events; these steps may then be repeated until the desired combination of transgenic events in a progeny is achieved. Back-crossing to a parental plant and out-crossing with a non-transgenic plant are also contemplated, as is vegetative propagation.

Methods of detecting the presence of cotton event Gh_CSM63718 in a sample of DNA derived from a cotton seed, plant, plant part, plant cell, progeny plant, or commodity product are provided. One method comprises: (i) contacting the sample with at least one probe specific to event Gh_CSM63718 DNA under conditions appropriate for DNA sequencing; (ii) performing a DNA sequencing reaction; and (iii) confirming that the nucleotide sequence comprises a nucleotide sequence specific for event Gh_CSM63718, of the transgenic insert comprised therein, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Another method of detecting the presence of cotton event Gh_CSM63718 in a sample of DNA derived from a cotton seed, plant, plant part, plant cell, progeny plant, or commodity product is provided. The method comprises: (a) contacting the sample with a DNA probe specific for event Gh_CSM63718 DNA; and (b) performing a sequencing reaction to produce a target sequence. The target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 17,736-17,737 of SEQ ID NO:10.

Another method of detecting the presence of cotton event Gh_CSM63718 in a sample derived from a cotton seed, plant, plant part or plant cell, progeny plant or commodity product comprises: (a) contacting the sample with a primer pair that is capable of producing an amplicon from event Gh_CSM63718 DNA under conditions appropriate for DNA amplification; (b) performing a DNA amplification reaction to produce a DNA amplicon; and (c) detecting the presence of the DNA amplicon. The DNA amplicon comprises a nucleotide sequence specific for event Gh_CSM63718, for example, at least one of (i) a 5′ junction sequence between flanking cotton genomic DNA and the transgenic insert of cotton event Gh_CSM63718, (b) a 3′ junction sequence between flanking cotton genomic DNA and the transgenic insert of cotton event Gh_CSM63718, (c) SEQ ID NO: 9, and (d) a fragment of SEQ ID NO: 9 comprising a sufficient length of contiguous nucleotides of SEQ ID NO: 9 to identify the sequence as a fragment of the transgenic insert of Gh_CSM63718. The presence of the DNA amplicon indicates the presence of cotton event Gh_CSM63718 in the sample. The amplicon should be one that is specific for event Gh_CSM63718, and comprises the junction at nucleotide positions 1000-1001, and/or nucleotide positions 17,736-17,737 of SEQ ID NO:10. Thus, for example, the amplicon can comprise a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 17,736-17,737 of SEQ ID NO:10. Alternatively or in addition, the amplicon can comprise a fragment of SEQ ID NO: 9 comprising a sufficient length of contiguous nucleotides of SEQ ID NO: 9 to identify the sequence as a fragment of the transgenic insert of cotton event Gh_CSM63718. The amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length.

The detection of a nucleotide sequence specific for event Gh_CSM63718 in the amplicon is determinative and/or diagnostic for the presence of the cotton event Gh_CSM63718 specific DNA in the sample. An illustrative primer pair that is capable of producing an amplicon from event Gh_CSM63718 DNA under conditions appropriate for DNA amplification is provided as SEQ ID NO:19 and SEQ ID NO:20. Other primer pairs may be readily designed by one of skill in the art to produce an amplicon diagnostic for cotton event Gh_CSM63718, wherein such a primer pair comprises at least one primer within the genomic region flanking the insert and a second primer within the insert, provided that any primer pair could be designed and used that produces an amplicon comprising a junction sequence and/or all or part of the insert or transgene sequence. Detection of an amplicon could be based on any suitable method, such as sequencing, determining fragment size or migration of the amplicon in a matrix or gel, or a hybridization-based method.

Another method of detecting the presence of cotton event Gh_CSM63718 in a sample derived from a cotton plant, plant part, plant cell, seed, progeny plant, or commodity product comprises: (i) contacting the sample with a DNA probe specific for event Gh_CSM63718 DNA; (ii) subjecting the sample and the DNA probe to stringent hybridization conditions; and (iii) detecting hybridization between the probe and the target DNA in the sample. An example of the sequence of a DNA probe that is specific for event Gh_CSM63718 is provided as SEQ ID NO:21. Other probes may be readily designed by one of skill in the art. Detection of probe hybridization to the DNA in the sample is diagnostic for the presence of cotton event Gh_CSM63718 specific DNA in the sample. Absence of hybridization is alternatively diagnostic of the absence of cotton event Gh_CSM63718 specific DNA in the sample.

Another method of detecting the presence of cotton event Gh_CSM63718 in a sample derived from a cotton plant, plant part, plant cell, seed, progeny plant, or commodity product comprises: (a) contacting the sample with at least one antibody specific for the PAT, TDO, DMO, EPSPS and PPO protein encoded by cotton event Gh_CSM63718; and (b) detecting binding of the antibody or antibodies to the protein(s) in the sample. The binding of the antibody or antibodies indicates the presence of cotton event Gh_CSM63718 in the sample.

Methods for determining the zygosity of the event and transgene with genomic DNA derived from at least one cotton plant, plant part, plant cell or seed comprising cotton event Gh_CSM63718 in a sample are provided. In one such method of determining the zygosity of a cotton plant, plant part, plant seed, or plant cell comprising cotton event Gh_CSM63718, the method comprises: (a) contacting a sample comprising DNA derived from the cotton plant, plant part, plant seed, or plant cell with a first primer set capable of producing a first amplicon diagnostic for the presence of cotton event Gh_CSM63718, and a second primer set capable of producing a second amplicon diagnostic for wildtype cotton genomic DNA not comprising cotton event Gh_CSM63718; (b) performing a nucleic acid amplification reaction; and (c) detecting the first amplicon and the second amplicon, wherein the presence of both amplicons indicates that the plant, plant part, seed or cell is heterozygous for cotton event Gh_CSM63718, and the presence of only the first amplicon indicates that the plant, plant part, seed, or cell is homozygous for cotton event Gh_CSM63718. The presence of only the second amplicon is diagnostic for the absence of event Gh_CSM63718 DNA in the sample. Illustrative sets of primer pairs are SEQ ID NO:19 and SEQ ID NO:20, which produce an amplicon diagnostic for event Gh_CSM63718; and SEQ ID NO:19 and SEQ ID NO:22, which produce an amplicon diagnostic for wildtype cotton genomic DNA not comprising event Gh_CSM63718. A set of probes can also be incorporated into such an amplification method to be used in a real-time PCR format using the primer pair sets described above. An illustrative set of probes are presented as SEQ ID NO:21 (diagnostic for the amplicon for the event Gh_CSM63718) and SEQ ID NO:23 (diagnostic for the amplicon for wildtype cotton genomic DNA not comprising event Gh_CSM63718).

Another method for determining the zygosity of a cotton plant, plant part, plant seed, or plant cell comprising cotton event Gh_CSM63718 comprises (a) contacting a sample comprising DNA derived from the cotton plant, plant part, plant seed, or plant cell with a probe set comprising at least a first probe that specifically hybridizes to cotton event Gh_CSM63718, and at least a second probe that specifically hybridizes to cotton genomic DNA that was disrupted by insertion of the heterologous DNA of cotton event Gh_CSM63718 but does not hybridize to cotton event Gh_CSM63718; and (b) hybridizing the probe set with the sample under stringent hybridization conditions. Detecting hybridization of only the first probe under the hybridization conditions is diagnostic for a cotton plant, plant part, seed or plant cell homozygous for cotton event Gh_CSM63718 and detecting hybridization of both the first probe and the second probe under the hybridization conditions is diagnostic for a cotton plant, plant part, seed, or plant cell heterozygous for cotton event Gh_CSM63718. Detecting hybridization of only the second probe under the hybridization conditions is diagnostic for the absence of event Gh_CSM63718 DNA in the sample. An illustrative probe set that can be used in the method is SEQ ID NO:21 and SEQ ID NO:23.

Yet another method for determining zygosity comprises (i) extracting a sample comprising DNA from at least one cotton plant, plant part, plant cell or seed; (ii) contacting the sample with a first primer pair that is capable of producing a first amplicon diagnostic for event Gh_CSM63718; (iii) contacting the sample with a second primer pair that is capable of producing a second amplicon of an internal standard known to be single-copy and homozygous in the cotton plant; (iv) contacting the sample with a probe set which contains at least a first probe that specifically hybridizes to the first amplicon, and at least a second probe that specifically hybridizes to the second amplicon; (v) performing a DNA amplification reaction using real-time PCR and determining the cycle thresholds (Ct values) of the first and second amplicons; (vi) calculating the difference (ACt) between the Ct value of the first amplicon and the second amplicon; and (vii) determining zygosity, wherein a ACt of about zero (0) indicates homozygosity of the event or inserted T-DNA, and a ACt of about one (1) indicates heterozygosity of the event or inserted T-DNA. Heterozygous and homozygous events are differentiated by a ACt value unit of approximately one (1). Given the normal variability observed in real-time PCR due to multiple factors such as amplification efficiency and ideal annealing temperatures, the range of “about one (1)” is defined as a ACt of 0.75 to 1.25, and the range of “about zero (0)” is defined as a ACt of −0.25 to 0.25 (or of 0.0 to 0.25 if the ACt is measured as an absolute value). Primer pairs and probes for the above method for determining zygosity can amplify and detect amplicons from the transgene or event DNA and the internal DNA standard.

A DNA construct is provided. The DNA construct comprises five expression cassettes, wherein the PAT expression cassette comprises in operable linkage i) a ribulose bisphosphate carboxylase/oxygenase (RuBisCO) activase gene promoter, and a leader sequence from Arabidopsis thaliana, ii) a codon-optimized phosphinothricin N-acetyltransferase (PAT) coding sequence from Streptomyces viridochromogenes, and iii) a 3′ UTR of a small heat shock protein (Hsp20) from Medicago truncatula; the TDO expression cassette comprises in operable linkage i) an enhancer from the strawberry vein banding virus (SVBV) fused to the promoter and 5′ UTR from a CAB1 (Chlorophyll A/B Binding Protein) gene from Cucumis melo, ii) a codon-optimized triketone dioxygenase (TDO) coding sequence from Oryza sativa, and iii) a 3′ UTR of a TMA7 (translation machinery associated 7) protein from Medicago truncatula; the DMO expression cassette comprises in operable linkage i) a polyubiquitin gene (UBQ10) promoter, a leader and an intron sequence from Arabidopsis thaliana, ii) an N-terminal chloroplast transit peptide coding sequence of APG6 (Albino and Pale Green 6) from Arabidopsis thaliana fused to a codon-optimized dicamba monooxygenase coding sequence from Stenotrophomonas maltophilia; and iii) a 3′ UTR of an aluminum-induced Sali3-2 protein from Medicago truncatula; the EPSPS expression cassette comprises in operable linkage i) an enhancer of the 35S gene from Figwort Mosaic Virus (FMV), ii) a promoter, a leader sequence, and an intron sequence of the elongation factor 1A gene (ELF1a) from Arabidopsis thaliana, iii) an N-terminal chloroplast transit peptide of granule bound starch synthase I from Triticum aestivum fused to a codon optimized 5-enolpyruvylshikimate-3-phosphate synthase gene (EPSPS) from Agrobacterium sp strain CP4, and iv) a 3′ UTR of a ribulose 1,5-bisphosphate carboxylase small subunit E9 (rbcS-E9) gene from Pisum sativum; and the PPO expression cassette comprises in operable linkage i) an enhancer derived from multiple enhancer sequences from Arabidopsis thaliana, ii) a promoter sequence designed from multiple promoter sequences from Arabidopsis thaliana, iii) an intron and 5′ UTR for a cytochrome C oxidase subunit VIa gene from Arabidopsis thaliana fused a 5′ UTR designed from multiple 5′ UTR sequences from Arabidopsis thaliana, iv) an N-terminal chloroplast transit peptide coding sequence of APG6 (Albino and Pale Green 6) from Arabidopsis thaliana, with monocot codon usage fused to the coding region of a protoporphyrinogen oxidase gene from Enterobacter cloacae with codons optimized for cotton, and v) a 3′ UTR from the fiber FbLate-2 gene from Gossypium barbadense. For example, the DNA construct can comprise SEQ ID NO: 9.

Expression of the PAT, DMO, TDO, EPSPS and PPO transgenes in transgenic cotton plants confers tolerance to glufosinate, dicamba, ß-triketone HPPD inhibitors, glyphosate and PPO herbicides. For example, plants, plant parts, plant cells or seeds containing or comprising cotton event Gh_CSM63718 are tolerant to PPO herbicides flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, and combinations of any thereof.

Any of the DNA constructs or transgenic inserts described herein can further comprise at its 5′ or 3′ end at least 50 contiguous nucleotides of SEQ ID NO:11 or SEQ ID NO:14, or at least 50 contiguous nucleotides of SEQ ID NO:12 or SEQ ID NO:15. Alternatively, any of the DNA constructs or transgenic inserts described herein can further comprise at its 5′ or 3′ end at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14, or at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or SEQ ID NO:15.

SEQ ID NOs:11 and 12 are 1,000 nucleotide sequences representing cotton genomic DNA that flanks the transgenic insert at the 5′ and 3′ ends of the insert in cotton event Gh_CSM63718, respectively. SEQ ID NO:11 and SEQ ID NO:12 have been validated by sequencing, as further described in Example 5 hereinbelow. SEQ ID NOs:14 and 15 are 5,000 nucleotide sequences representing cotton genomic DNA that flanks the transgenic insert at the 5′ and 3′ ends of the insert, respectively. Nucleotides 4,001-5,000 of SEQ ID NO:14 are identical to nucleotides 1-1,000 of SEQ ID NO:11. The remaining nucleotides of SEQ ID NO:14 (nucleotides 1-4,000) are based on the genomic sequence of the TM-1 cotton cultivar (Gossypium hirsutum version 3.1, DOE JGI Genome Portal). Similarly, nucleotides 1-1000 of SEQ ID NO:15 are identical to nucleotides 1-1,000 of SEQ NO:12. The remaining nucleotides of SEQ ID NO:15 (nucleotides 1,001-5,000) are based on the genomic sequence of the TM-1 cotton cultivar.

The contiguous nucleotides of SEQ ID NO:11 or SEQ ID NO:14 at the 5′ end of the DNA construct or transgenic insertion may be immediately adjacent to and upstream (on the 5′ end) of the transgenic insertion, or may not be immediately adjacent to, but further upstream (on the 5′ end) and within about 5000 nucleotides, within about 3000 nucleotides, or within about 1000 nucleotides of the transgenic insertion. Likewise, the contiguous nucleotides of SEQ ID NO:12 or SEQ ID NO:15 at the 3′ end of the DNA construct or transgenic insertion may be immediately adjacent to and downstream (on the 3′ end) of the transgenic insertion, or may not be immediately adjacent to but further downstream (on the 3′ end) and within about 5000 nucleotides, within about 3000 nucleotides, or within about 1000 nucleotides of the transgenic insertion. Illustrative examples of sequences comprising 50 contiguous nucleotides of SEQ ID NO:11 are provided in SEQ ID NOs:58-77. Illustrative examples of 50 contiguous nucleotides of SEQ ID NO:12 are provided in SEQ ID NOs:128-147. Illustrative examples of 50 contiguous nucleotides of SEQ ID NO:14 are provided in SEQ ID NOs:58-127. Illustrative examples of 50 contiguous nucleotides of SEQ ID NO:15 are provided in SEQ ID NOs:128-197. However, any sequence comprising at least 50 contiguous nucleotides of SEQ ID NO:11 or SEQ ID NO:14, or at least 50 contiguous nucleotides of SEQ ID NO:12 or SEQ ID NO:15 is within the scope of the present disclosure.

In addition, a DNA construct comprising five expression cassettes of PAT, DMO, TDO, EPSPS and PPO is provided. The DNA construct further comprises at the 5′ and/or 3′ end of the construct (i) at least 50 contiguous nucleotides of SEQ ID NO:11 or SEQ ID NO:14; and/or (ii) at least 50 contiguous nucleotides of SEQ ID NO:12 or SEQ ID NO:15.

A further DNA construct is provided. The DNA construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the full length of SEQ ID NO: 9. The DNA construct further comprises at the 5′ and/or 3′ end of the construct (i) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or 14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or 15.

For example, any of the DNA constructs can comprise at the 5′ end of said construct one or more nucleotide sequences selected from SEQ ID NOs:58-127. Alternatively or in addition, any of the DNA constructs can comprise at the 3′ end of said construct one or more nucleotide sequences selected from SEQ ID NOs:128-197.

Cotton plants, plant cells, plant parts, and plant seeds comprising any of the DNA constructs described herein are also provided.

Also provided are cotton plants, plant cells, plant parts, and plant seeds comprising a recombinant DNA construct integrated in chromosome 21, wherein the recombinant DNA construct confers tolerance to at least one herbicide selected from the group consisting of glufosinate, dicamba, a ß-triketone HPPD inhibitor, glyphosate, a PPO herbicide, and combinations of any thereof. The recombinant DNA construct is integrated in a position of said chromosome flanked by at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or 14 and at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or 15. The at least 50 contiguous nucleotides of SEQ ID NO:11 or 14 can comprise one or more nucleotide sequences selected from SEQ ID NOs:58-127, and the at least 50 contiguous nucleotides of SEQ ID NO:12 or 15 can comprise one or more nucleotide sequences selected from SEQ ID NOs:128-197.

Methods of improving tolerance to herbicides are provided. The methods comprise: i) inserting a DNA construct comprising a PAT expression cassette, a DMO expression cassette, a TDO expression cassette, an EPSPS expression cassette, and a PPO expression cassette, as described herein, into the genome of a cotton cell, ii) generating a cotton plant from the cotton cell; and iii) selecting a cotton plant comprising the DNA construct. The selecting can comprise treating the cotton cell or plant with an effective amount of at least one herbicide selected from the group consisting of glufosinate, dicamba, ß-triketone HPPD inhibitors, glyphosate, PPO herbicides, and combinations of any thereof.

Transgenic plants produced by the methods described herein comprise a unique combination of five expression cassettes, each with a unique combination of expression elements, for optimal expression of the transgenes. Furthermore, transgenic plants produced by the methods as described herein acquire tolerance to at least one herbicide selected from the group consisting of glufosinate, dicamba, ß-triketone HPPD inhibitors, glyphosate, PPO herbicides, and combinations of any thereof. Selecting the regenerated plant comprising the DNA construct may be done using DNA or protein detection methods as described herein. Alternatively or additionally, selecting may comprise treating the transgenic plant or plant cell with an effective amount of at least one herbicide selected from the group consisting of glufosinate, dicamba, ß-triketone HPPD inhibitors, glyphosate, PPO herbicides, and combinations of any thereof.

Methods of controlling, preventing, or reducing the development of herbicide-tolerant weeds are provided. The methods comprise: a) cultivating in a crop growing environment a cotton plant comprising a DNA construct or transgenes of the present disclosure or event Gh_CSM63718 that provides tolerance to at least one herbicide selected from the group consisting of glufosinate, dicamba, ß-triketone HPPD inhibitors, glyphosate, PPO herbicides, and combinations of any thereof, and b) applying to the crop growing environment at least one herbicide selected from the group consisting of glufosinate, dicamba, ß-triketone HPPD inhibitors, glyphosate, PPO herbicides, and combinations of any thereof, wherein the cotton plant is tolerant to the at least one herbicide selected from the group consisting of glufosinate, dicamba, ß-triketone HPPD inhibitors, glyphosate, PPO herbicides, and combinations of any thereof.

In any of the methods of controlling, preventing, or reducing the development of herbicide-tolerant weeds, the transgenes provide tolerance to the herbicides with at least three different herbicide modes of action and are present at a single genomic location in the cotton plant.

The PAT coding sequence and its corresponding amino acid sequence from Streptomyces viridochromogenes are provided as SEQ ID NO:27 and SEQ ID NO:207, respectively. The TDO coding sequence and its corresponding amino acid sequence from Oryza sativa are provided as SEQ ID NO:33 and SEQ ID NO:208, respectively. The DMO coding sequence and its corresponding amino acid sequence from Stenotrophomonas maltophilia are provided as SEQ ID NO:39 and SEQ ID NO:209, respectively. The EPSPS coding sequence and its corresponding amino acid sequence from Agrobacterium tumefaciens CP4 strain are provided as SEQ ID NO:46 and SEQ ID NO:210, respectively. The PPO coding sequence and its corresponding amino acid sequence from Enterobacter cloacae are provided as SEQ ID NO:50 and SEQ ID NO:211, respectively.

The cotton plants, plant seeds, plant parts, or plant cells produced by the preceding methods are tolerant to at least one herbicide in a field, selected from the group consisting of benzoic acid auxins, inhibitors of glutamine synthetase, ß-triketone HPPD inhibitors, inhibitors of EPSPS, PPO inhibitors, and combinations of any thereof. An example of benzoic acid auxins includes, but is not limited to, dicamba. An example of glutamine synthetase inhibitors includes, but is not limited to, glufosinate. Illustrative examples of ß-triketone HPPD inhibitors include, but are not limited to, mesotrione, benzobicyclon (BBC), tembotrione, sulcotrione, tefuryltrione, and combinations of any thereof. An example of the inhibitor of 5-enolpyruvylshikimate-3-phosphate synthase (EPSPS) is glyphosate. Illustrative examples of PPO herbicides include, but are not limited to, flumioxazin, epyrifenacil, lactofen, acifluorfen, pyraflufen, pyraflufen-ethyl, oxadiazon, butafenacil, carfentrazone-ethyl, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, trifludimoxazin, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, and 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate, and combinations of any thereof.

The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after cotton seed planting), or both. For example, a method for controlling the development of herbicide resistance in weeds is provided that consists of planting seed comprising cotton event Gh_CSM63718 in an area and applying an herbicidally effective amount over the growing season of one or more PPO herbicides alone or in any combination or in combination with other herbicides, for the purpose of controlling the development of herbicide resistance in weeds in the area. Such application of herbicide(s) may be pre-planting (any time prior to planting seed comprising cotton event Gh_CSM63718, including for bum-down purposes, that is application to emerging or existing weeds prior to seed plant), pre-emergence (any time after seed comprising cotton event Gh_CSM63718 is planted and before plants comprising cotton event Gh_CSM63718 emerge), or post-emergence (any time after plants comprising cotton event Gh_CSM63718 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Also provided are methods of reducing loci for cotton breeding by inserting and expressing five transgenes within one DNA construct at one genomic locus in a cotton plant that provides tolerance to herbicides having different herbicide modes of action, each being different from one another. The transgenes for tolerance to different herbicide modes of action encode PAT, TDO, DMO, EPSPS, and PPO. These transgenes provide a commercial level of tolerance to at least one herbicide in a field, such as glufosinate, mesotrione, dicamba, glyphosate, a PPO herbicide, and combinations of any thereof. As used herein, the term “commercial level” in reference to an herbicide refers to the recommended commercial rate (1×) for herbicide application for a specific herbicide. For example, a 1× rate of dicamba is 0.5 lb/acre pre-emergence and post-emergence; a 1× rate of glufosinate is 0.53 lb/acre post-emergence; a 1× rate of mesotrione is 0.2-lb/acre pre-emergence, and 0.094 lbs ai/acre post-emergence; a 1× rate of glyphosate is 1.125 lb ae/acre post-emergence; and a 1× rate of epyrifenacil PPO herbicide is 0.0178 lb/acre post-emergence. As used herein, “commercial level tolerance” refers to tolerance to one or more herbicides at the recommended commercial rates or higher as a result of transgene expression from one or more expression cassettes in plants comprising event Gh_CSM63718.

Cotton event Gh_CSM63718 with the unique characteristics such as stable integration and expression of the PAT, TDO, DMO, EPSPS and PPO transgenes, consistent and superior combinations of efficacy, including herbicide tolerance and agronomic performance, in and across multiple environment conditions in different geographies can be bred or introgressed into elite lines or varieties by conventional breeding methods, and maintained over subsequent generations. Furthermore, since the event comprises five transgenes integrated at a single locus, the methods of the present disclosure allow PAT, TDO, DMO, EPSPS and PPO to segregate as a single locus, which will allow for rapid trait integration of the multiple transgenes on segregating material, saving time and resources in a breeding program and enabling rapid development of lines, compared to cases where the individual transgenes are inserted at two or more loci, necessitating tedious and laborious multiple crosses over multiple generations to select for plants comprising the multiple genes. The newly introgressed or integrated DNA molecule or polynucleotide of event Gh_CSM63718 comprising SEQ ID NO:9 and/or SEQ ID NO:10 will maintain the expression characteristics of the transgene, and the genomic flanking sequences and chromosomal location, where it will confer tolerance to glufosinate, ß-triketone HPPD inhibitors, glyphosate and PPO herbicides.

### Deposit Information

A deposit of a representative sample of cotton seed comprising event Gh_CSM63718 has been made on Nov. 17, 2023, according to the Budapest Treaty with the American Type Culture Collection (ATCC) Patent Repository having an address at 10801 University Boulevard, Manassas, Virginia, 20110, USA. The ATCC Patent Deposit Designation (accession number) for seeds comprising cotton event Gh_CSM63718 is Accession No. PTA-127638. Access to the deposits will be available during the pendency of the application to the Commissioner of Patents and Trademarks and persons determined by the Commissioner to be entitled thereto upon request. Upon issuance of the patent, all restrictions upon availability to the public will be irrevocably removed. The deposit will be maintained in the depository for a period of thirty (30) years, or five (5) years after the last request, or for the effective life of the patent, whichever is longer, and will be replaced as necessary during that period.

### EXAMPLES

The following examples are included to more fully describe the invention. Summarized are the construction and testing of 95 initial proof-of-concept transformation constructs containing various combinations of expression elements and transgenes/variants, leading to the construction and testing of four commercial transformation constructs, the production of over 14,662 unique transformation events from the four commercial transformation constructs, screening of all healthy regenerated plants for single copy insertion of the transgene followed by controlled environment spray test of the single copy events for herbicide tolerance, and the analysis of individual plants over multiple seasons through rigorous molecular characterization, efficacy and agronomic testing both in a controlled environment and in field trials, leading to the creation, identification, and ultimate selection of cotton event Gh_CSM63718 (FIG. 3).

The Examples illustrate certain embodiments of the present disclosure. It should be appreciated by those of skill in the art that many modifications can be made in the specific examples which are disclosed and still obtain a similar result. Certain agents which are both chemically and physiologically related may be substituted for the agents described herein while achieving the same or similar results. All such substitutions and modifications apparent to those skilled in the art are deemed to be within the scope of the invention.

### Example 1: Expression Cassette Testing, Construct Design, Plant Testing and Construct Selection

Transgene expression and performance in transgenic plants may be influenced by many factors. These include but are not limited to: 1) the expression elements used to drive the expression of each transgene in the expression cassette, and their interactions among themselves and with the transgene; 2) efficient targeting of the transgenic protein to the site of action, such as in the plastids; 3) the relative position and orientation of different expression cassettes when the transgenic insert comprises multiple expression cassettes, each carrying a different transgene conferring a distinct trait or function; and 4) the genomic location of the transgenic insertion, also known as positional effect. A commercially useful transgenic event requires that each of the transgene(s) in the transgenic insert express in the manner necessary for that trait or function to be successful, e.g., transgene expression and performance across different tissues and developmental stages, in various germplasms, and under different growth conditions.

For these reasons, the development of a transgenic cotton plant conferring herbicide tolerance without any negative effect on agronomics, yield or stacking viability requires extensive research, development, and analysis. It is often necessary to create and screen a large number of constructs and transformation events in order to identify combinations of expression elements for constructs (the lead constructs), and then an event (the lead event), which demonstrates optimal expression and performance of the transgenes without phenotypic and agronomic off-types such as yield drag. Prior to such studies, it is not possible to determine whether a particular beneficial event phenotype or performance can be obtained.

This example describes testing and selection of herbicide tolerance genes of interest, 5′ and 3′ expression elements, chloroplast transit peptide sequences, transgene cassette configurations (i.e., relative orientations and positions within the T-DNA) in the initial proof-of-concept (POC) and early development stages over a three year period to identify and select constructs with desirable combinations for commercial transformation and event selection to achieve tolerance to different herbicides.

A total of 95 different constructs were designed and tested containing six PPO genes/variants, four TDO variants, one variant each of the PAT, EPSPS and DMO genes, together with different 5′ UTRs, chloroplast targeting sequences, and 3′UTRs as summarized in Table 4. Each construct varied with respect to the coding sequences for the herbicide tolerance proteins and the transcriptional regulatory elements, and had a unique configuration, varying by expression cassette composition (herbicide tolerance proteins and expression elements), orientation, and position. These constructs were transformed into cotton mature seed-derived embryo explants through Agrobacterium-mediated transformation using methods known in the art. Each construct also contained an aadA cassette as a selectable marker. In addition, four CRE cassettes were designed and tested for auto excision of the selectable marker.

Table 4 provides a summary of the proof-of-concept constructs. A total of 10,308 R0 transgenic events were produced from the 95 constructs, each made by a random insertion of the transgenic insert into the cotton genome. R0 plants were regenerated and rooted plants with normal phenotypic characteristics were transferred to soil for growth and further assessment. The events went through initial molecular screening to remove those with undesirable molecular characteristics such as multiple copies of the transgenic insert or presence of the transformation vector backbone sequence. A total of 1,645 transgenic events were advanced for herbicide tolerance efficacy testing. These plants were tested in the greenhouse for tolerance to glyphosate at 2.25 lb ai/acre (Roundup PowerMax®), and/or glufosinate at 1.06 ai/acre (Liberty®), and/or dicamba at 1 lb ai/acre (Clarity®), and/or mesotrione at 0.2 lb ai/acre (Callisto®), and/or a tank mixture of the above mentioned four herbicides, and/or epyrifenacil at 0.036 lb/acre (Rapidicil©), sprayed at the V3 growth stage followed by early bloom stage, assessed visually for injury 14 days after the herbicide sprays, with 0% representing no injury and 100% representing complete plant death. Events with >15% injury were discarded. From the greenhouse spray results, a total of 269 events were advanced to R2 generation for field testing.

TABLE 4
Summary of Proof-of-Concept Constructs

| Expression Cassette | PPO | TDO | PAT | EPSPS | DMO |
|---|---|---|---|---|---|
| # Gene variants | 6 | 4 | 1 | 1 | 1 |
| # Promoters/leaders/introns | 15 | 6 | 6 | 2 | 4 |
| # CTPs | 9 | — | — | 2 | 2 |
| # 3′ UTRs | 9 | 3 | 5 | 2 | 3 |

| | |
|---|---|
| # Constructs | 35 (18 for 4-cassette constructs; 17 for |
| | 5-cassette constructs) |

Based on these results, top performing individual expression cassettes were identified, each containing individual transgenes driven by combinations of expression elements. From these individual expression cassettes, four different commercial transformation constructs were designed, three containing five expression cassettes (encoding PAT, TDO, DMO, EPSPS and PPO, respectively) and one containing four expression cassettes (encoding PAT, TDO, DMO and EPSPS). The constructs also contained an aadA cassette for selection of transgenic events, and a Cre cassette for excision/removal of the aadA and Cre cassettes, and the ISR1 (an intergenic sequence region) in the progenies. These four constructs contained different expression cassette combinations, varying by expression elements, and the relative position and orientation of the transgene cassettes. The transgene cassettes in the T-DNA were arranged as follows in the four constructs: (1) “Left Border-Lox-aadA (>)—Cre (>)—ISR1-Lox-PAT (<)<TDO (>)—DMO (>)—EPSPS (>)—ISR2-PPO (<)<Right Border” in construct GhHT4-1; (2) “Left Border-Lox-aadA (>)—Cre (>)—ISR1-Lox-PPO (>)—PAT (<)<TDO (>)—DMO (<)<EPSPS (>)—Right Border” in construct GhHT4-2; (3) “Left Border-Lox-aadA (>)—Cre (>)—ISR1-Lox-PPO (>)—DMO (<)<TDO (>)—PAT (>)—EPSPS (>)—Right Border” in construct GhHT4-3; and (4) “Left Border-Lox-aadA (>)—Cre (>)—ISR1-Lox-PAT (<)<TDO (>)—DMO (>)—EPSPS (>)—Right Border” in construct GhHT4-4. An arrow following the specific cassette shows the orientation of the cassette immediately before the arrow, with the left pointing arrow (<) representing the orientation of the cassette as 3′ UTR-coding sequence-promoter and 5′ UTR, and with the right pointing arrow (>) representing the orientation of the cassette as promoter and 5′ UTR-coding sequence-3′ UTR. The aadA expression cassette contained the aadA gene (also known as Tn7 adenylyltransferase (AAD(3”))) from E. coli under the control of a constitutive promoter, and targeted the aadA protein to the chloroplast for selection of transgenic events resistant to spectinomycin and streptomycin. The Cre expression cassette encodes the Cre recombinase from Enterobacteria phage P1, under the control of an anther specific promoter, for removal of the aadA and Cre cassettes, and ISR1 in the progenies.

The four- or five-expression cassettes constructs were cloned into plant transformation vectors, and introduced into cotton mature seed-derived embryo explants through Agrobacterium-mediated transformation using methods known in the art. A total of 14,662 unique transformation events were produced, each made by a random insertion of the transgenic insert into the cotton genome. R0 plants were subsequently regenerated from the transgenic events. Rooted plants with normal phenotype were transferred to soil for growth and further assessment.

Healthy looking, phenotypically normal R0 events were assayed for transgenic insert copy number. The results in Table 5 show that a total of 4,673 unique events containing a single copy of the intact transgenic insert were identified. These events were assessed in the greenhouse for herbicide tolerance. The plants were sprayed with herbicides at V3 stage as described above and were assessed visually for injury 14 days treatment, with 0% representing no injury and 100% representing complete plant death. Events with >10% injury were discarded. Herbicide treatments under greenhouse conditions were applied using a track-mounted sprayer in enclosed cabinet calibrated to deliver 15 gallons per acre (GPA) using Teejet® TTI nozzles with water as the herbicide carrier.

The results are summarized in Table 5. From a total of 1,354 events passing the greenhouse spray test, 527 events produced ≥300 R1 seeds per event. After further molecular characterization to confirm that the events contained a single copy of the intact transgenic insert without the presence of the transformation vector backbone, 287 events were discarded, and 240 events remained.

Based on the results of greenhouse spray test and molecular characterization, 80 transgenic events from the four commercial transformation constructs along with 269 events from 45 POC transformation constructs were advanced to field trials for evaluation of the events from the constructs and/or expression cassette(s).

TABLE 5
Summary of Events from the Four Transformation Constructs

| | # R0 | # R0 Events | # R0 Events | # R0 Events |
|---|---|---|---|---|
| | Events | Single Copy | with <10% | Producing |
| Construct | Assayed | for Spray | Injury | R1 Seeds |
| GhHT4-1 | 6355 | 1408 | 405 | 121 |
| GhHT4-2 | 2770 | 1200 | 320 | 135 |
| GhHT4-3 | 2933 | 1037 | 310 | 147 |
| GhHT4-4 | 2604 | 1028 | 319 | 124 |
| Total | 14662 | 4673 | 1354 | 527 |
| | | | | (240 passed |
| | | | | molecular |
| | | | | criteria |

### Example 2: Field Trials

Field trials were conducted over multiple years and across many locations in different North America geographies to evaluate the performance of the constructs/events, and for selecting the best performing construct/event. The field trials included efficacy trials for herbicide tolerance, and agronomic trials for yield performance of the events. The performance of many individual plants for each event in each field trial was analyzed as a set. Each event was thus represented by many individual plants. This allowed the performance of each event to be analyzed under many different conditions, in different locations and geographies, and for a variety of properties. Field trials were conducted on homozygous plants to assess trait efficacy for tolerance to commercial rates of glufosinate, ß-triketone HPPD inhibitors, dicamba, glyphosate and PPO herbicides, and agronomic performance.

The first year and second year field trials included proof of concept constructs to evaluate performance at the construct level. The trials were designed to detect 10% yield difference at an alpha 0.05. The third year field trials focused on the four commercial constructs but also included some proof of concept constructs to identify lead constructs to be advanced for further field testing designed to detect 5% yield difference at an alpha of 0.05.

### First Year Field Trials

The first-year efficacy and agronomic field trials were conducted on R² homozygous events from POC constructs, including five single-cassette constructs encoding TDO or CP4 EPSPS or PAT, respectively, one three-cassette construct encoding CP4 EPSPS, PAT and DMO, twelve four-cassette constructs encoding EPSPS, PAT, TDO and DMO under the control of different combinations of regulatory sequences and in different relative orientations and/or positions. In the agronomy trials, the events were tested at 6 locations with 3 replications per location. In the efficacy trials, the events were tested at 2 locations with 3 replications per location. Both efficacy and agronomic trials used a randomized complete block design (RCBD). The efficacy trials included five herbicide treatments: 1) glyphosate at 2.25 lb ai/acre (Roundup Powermax®) 2) glufosinate at 1.06 lb ai/acre (Liberty®); 3) dicamba at 1 lb ai/acre (Clarity®); 4) mesotrione at 0.2 lb ai/acre (Callisto®); and 5) a tank mix of the above mentioned four herbicides at the same rates. Herbicides were applied at V3 growth stage followed by early bloom stage using a tractor-mounted or backpack sprayer calibrated to deliver 15 gallons per acre (GPA) with water as the herbicide carrier using Teejet Air Induction (TTI) nozzles. Herbicide injury was evaluated by visual inspection 14 days after herbicide applications on a scale of 0 to 100 with “0” being no visible crop injury and “100” being complete crop injury or death.

Plots were two rows by forty feet long with a ten-foot alley with 30-inch row spacing and seeded at 235 seeds per plot. Trial maintenance was designed to optimize total seed yield. All data from both agronomic and efficacy trials were collected at construct level and subjected to analysis of variance and means separated at LSD at alpha 0.05. Statistical analysis was automated using ASReml software for mixed model fitting.

Constructs were prioritized for further testing based on molecular assessment, yield performance, and crop injury. In the agronomic trials, yield (total seed cotton) at the construct level was compared to that of a wildtype control (using the same germplasm as the transgenic events) and a commercial check (Bollgard II® Xtend Flex®, which was also used as a commercial check in the subsequent years' field trials). Constructs showing >10% yield reduction compared to controls in the agronomic trials were discontinued.

Results of the efficacy and agronomy trials for the POC constructs are summarized in Table 6. Also summarized is protein expression data by ELISA using leaf tissues collected from V6 stage plants. All constructs showed excellent crop response in the efficacy trials even though a wide range of protein expression levels was observed among these constructs. Constructs POC-6 to POC-11 showed significantly reduced yield in the agronomics trials when compared to the controls. The transgene cassette information (such as the genetic elements) and the configuration of the transgene cassettes (such as the relative position and/or orientation of the cassettes) were used to guide the design of five transgenic cassette constructs and the commercial constructs.

TABLE 6
Summary of First-Year Efficacy and Agronomic Trial Results

| | | | | | | | | | | | Yield |
|---|---|---|---|---|---|---|---|---|---|---|---|
| | | | | | | % Average Crop Injury | | | | | (total |
| | | | | | | 2x | 2x | 2x | 2x | 2 | Seed |
| | | V6 Leaf Protein (ppm) | | | | Glyphosate | Glufosinate | Dicamba | Mesotrione | Tank | Cotton |
| Construct | Transgene | EPSPS | DMO | TDO | PAT | (Powermax) | (Liberty) | (Clarity) | (Callisto) | Mix | (lbs/acre)) |
| POC-1 | TDO | N/A | N/A | 26.16 | N/A | N/A | N/A | N/A | 0.5 | N/A | 4798.27 ± |
| | | | | | | | | | | | 270 |
| POC-2 | TDO | N/A | N/A | 25.1 | N/A | N/A | N/A | N/A | 0.5 | N/A | 5104.46 ± |
| | | | | | | | | | | | 95 |
| POC-3 | EPSPS | 102.4 | N/A | N/A | N/A | 6.7 | N/A | N/A | N/A | N/A | 5302.18 ± |
| | | | | | | | | | | | 97 |
| POC-4 | PAT | N/A | N/A | N/A | 1.9 | N/A | 2.75 | N/A | N/A | N/A | 5595.23 ± |
| | | | | | | | | | | | 95 |
| POC = 5 | EPSPS | 203 | N/A | N/A | N/A | 6.1 | N/A | N/A | N/A | N/A | 5172.21 ± |
| | | | | | | | | | | | 113 |
| POC-6 | EPSPS, | 205 | 189 | N/A | 5.32 | 2.7 | 2 | 1.0 | N/A | N/A | 4414.44 ± |
| | PAT, DMO | | | | | | | | | | 104 |
| POC-7 | EPSPS, | 140.53 | 211.11 | 59.91 | 11.89 | 2.5 | 3.7 | 3.2 | 2.5 | 12.7 | 4534.45 ± |
| | PAT, TDO, | | | | | | | | | | 99 |
| | DMO | | | | | | | | | | |
| POC-8 | EPSPS, | 215.02 | 416.64 | 48.27 | 11.62 | 12.5 | 2.2 | 2.0 | 2.5 | 11.5 | 4080.41 ± |
| | PAT, TDO, | | | | | | | | | | 95 |
| | DMO | | | | | | | | | | |
| POC-9 | EPSPS, | 230.72 | 362.92 | 40.17 | 5.88 | 2.5 | 3.0 | 2.0 | 1.7 | 9.5 | 3969.35 ± |
| | PAT, TDO, | | | | | | | | | | 120 |
| | DMO | | | | | | | | | | |
| POC-10 | EPSPS, | 284 | 225.94 | 68.36 | 9.18 | 3.0 | 2.7 | 2.0 | 2.5 | 10.2 | 4317.94 ± |
| | PAT, TDO, | | | | | | | | | | 97 |
| | DMO | | | | | | | | | | |
| POC-11 | EPSPS, | 224.11 | 296.24 | 66.29 | 12.39 | 3.2 | 3.0 | 2.0 | 2.7 | 10.2 | 4400.21 ± |
| | PAT, TDO, | | | | | | | | | | 99 |
| | DMO | | | | | | | | | | |
| POC-12 | EPSPS, | 115.61 | 101.11 | 11.45 | 12.46 | 2.2 | 2.0 | 2.2 | 5.5 | 14.2 | 4797.23 ± |
| | PAT, TDO, | | | | | | | | | | 101 |
| | DMO | | | | | | | | | | |
| POC-13 | EPSPS, | 161.12 | 103.96 | 45.36 | 10.97 | 3.0 | 2.2 | 2.7 | 2.2 | 13.0 | 4712.33 ± |
| | PAT, TDO, | | | | | | | | | | 113 |
| | DMO | | | | | | | | | | |
| POC-14 | EPSPS, | 186.5 | 122.38 | 61.64 | 12.6 | 3.5 | 4.2 | 3.2 | 3.0 | 11.7 | 4920.62 ± |
| | PAT, TDO, | | | | | | | | | | 110 |
| | DMO | | | | | | | | | | |
| POC-15 | EPSPS, | 251 | 150.91 | 27.23 | 6.12 | 4.2 | 4.5 | 3.0 | 2.7 | 13.5 | 5141.25 ± |
| | PAT, TDO, | | | | | | | | | | 95 |
| | DMO | | | | | | | | | | |
| POC-16 | DMO, | 620.24 | 92.63 | 67.77 | 5.83 | 2.7 | 2.0 | 2.7 | 2.2 | 9.2 | 4887.05 ± |
| | PAT, TDO, | | | | | | | | | | 116 |
| | EPSPS | | | | | | | | | | |
| POC-17 | EPSPS, | 296.46 | 11.32 | 61.5 | 16.04 | 2.7 | 3.2 | 2.7 | 2.2 | 12.5 | 4927.24 ± |
| | DMO, | | | | | | | | | | 104 |
| | TDO, PAT | | | | | | | | | | |
| POC-18 | DMO, | 502.92 | 122.38 | 64.41 | 18.47 | 3.2 | 2.7 | 2.7 | 2.7 | 10.7 | 4527.91 ± |
| | PAT, TDO, | | | | | | | | | | 116 |
| | EPSPS | | | | | | | | | | |

| | |
|---|---|
| Commercial check | 5348.91 ± |

| | 46 |
|---|---|
| Wild Type Control | 5432.26 ± |
| | 73 |

### Second Year Field Trials

The second-year field trials were conducted on R2-R5 generation homozygous events from POC constructs depending on the progression of the constructs in the pipeline and on the availability of seeds. The constructs included ten single-cassette constructs encoding DMO or TDO or EPSPS or PAT or one of three different PPO variants, eight four-cassette constructs encoding EPSPS, PAT, TDO, and DMO under the control of different combinations of regulatory sequences and in different relative orientations and/or positions, seven five-cassette constructs encoding EPSPS, PAT, TDO, DMO, and one of two different PPO variants under the control of different combinations of regulatory sequences and in different relative orientations and/or positions. Both efficacy and agronomic trials used a randomized complete block design (RCBD). In the agronomy trials, the R3 or higher generation events were tested at 7 locations with 3 replications per location, whereas the R2 generation events were tested at 4 locations with three replications per location.

In the efficacy trials, the events were tested at 3-6 locations with 3 replications per location. There were seven herbicide treatments, all at 2× recommended commercial rates: 1) glyphosate (Roundup Powermax®: 2) glufosinate (Liberty®); 3) dicamba (XtendiMax®); 4) mesotrione (Callisto®); 5) epyrifenacil (Rapidicil®), 6) a 4-way tank mix of 2× each of glyphosate, dicamba, mesotrione and glufosinate, and 7) a 5-way tank mix of 2× each of glyphosate, dicamba, mesotrione, glufosinate and epyrifenacil. Herbicides were applied at V3 growth stage followed by early bloom stage using a tractor-mounted or backpack sprayer calibrated to deliver 15 gallons per acre (GPA) with water as the herbicide carrier using Teejet Air Induction (TTI) nozzles. Herbicide injury was evaluated by visual inspection 14 days after herbicide applications on a scale of 0 to 100 with “0” being no visible crop injury and “100” being complete crop injury or death.

Plots were two rows by forty feet long with a ten-foot alley with 30-inch row spacing and seeded at 235 seeds per plot. Trial maintenance was designed to optimize total seed yield. All data from both agronomic and efficacy trials were collected at construct level and subjected to analysis of variance and means separated at LSD at alpha 0.05. Statistical analysis was automated using ASReml software for mixed model fitting.

The results of the second-year efficacy and agronomics trials are summarized in Table 7. Also summarized is protein expression data by ELISA using R2 (generation) trial leaf tissues collected from V6 stage plants (Table 8). In Table 7, “Gly” refers to glyphosate, “Dic” refers to dicamba, “Epy” refers to epyrifenacil, “Meso” refers to mesotrione, “Glu” refers to glufosinate, and PPO_H_N10, PPO_H_N40 and PPO_H_N90 refer to the three PPO variants. The commercial check was Bollgard II® Xtend Flex®. In Table 8, the column “#Events (Gen)” refers to the number of events tested for each construct, and generation designation of the plants. In both Tables 7 and 8, “No data” means either the data was not collected, or data was missing due to various reasons; “N/A” means not applicable. Most constructs showed excellent crop response to single herbicide treatments in the efficacy trials even though a large protein expression range among these constructs was observed. Several four-cassette constructs tested showed significant crop injuries following application of the 4-way tank mix herbicides. Expression of the transgenes in these events did not have any negative impact on total seed cotton yield.

TABLE 7
Summary of Second-Year Efficacy and Agronomics Trial Results

| | | % Averaged Crop Injury | | | | | | | |
|---|---|---|---|---|---|---|---|---|---|
| | | | | | | | 4-Way | 5-Way | Yield |
| Construct | Transgene | Gly | Dic | Epy | Meso | Glu | Tank Mix | Tank Mix | (lbs/acre) |
| CONTROL | Commercial Check | 6 | 3 | N/A | N/A | 7 | N/A | N/A | 2607.8 ± 30 |
| POC-19 | DMO | N/A | 3 | N/A | N/A | N/A | N/A | N/A | 2403.9 ± 238 |
| POC-5 | EPSPS | 8 | N/A | N/A | N/A | N/A | N/A | N/A | 3262 ± 600 |
| POC-20 | EPSPS | 3 | N/A | N/A | N/A | N/A | N/A | N/A | 3007 ± 753 |
| POC-2 | TDO | N/A | N/A | N/A | 4 | N/A | N/A | N/A | 3368 ± 1455 |
| POC-4 | PAT | N/A | N/A | N/A | N/A | 4 | N/A | N/A | 2974 ± 355 |
| POC-21 | PPO_H_N40 | N/A | N/A | 10 | N/A | N/A | N/A | N/A | 2991 ± 899 |
| POC-22 | PPO_H_N10 | N/A | N/A | 2.3 | N/A | N/A | N/A | N/A | 3136 ± 02 |
| POC-30 | PPO_H_N10 | N/A | N/A | 8 | N/A | N/A | N/A | N/A | 2429 ± 204 |
| POC-31 | PPO_H_N10 | 5 | 3 | 3 | 5 | 2 | No | No | 2489 ± 309 |
| | | | | | | | data | data | |
| POC-34 | PPO_H_N90 | N/A | N/A | 4 | N/A | N/A | N/A | N/A | 2436 ± 404 |
| POC-7 | EPSPS, PAT, TDO, DMO | 8 | 5 | N/A | 10 | 8 | 40 | N/A | 2964 ± 520 |
| POC-9 | EPSPS, PAT, TDO, DMO | 3 | 2 | N/A | 3 | 5 | 10 | N/A | 2967 ± 325 |
| POC-14 | EPSPS, PAT, TDO, DMO | 7 | 5 | N/A | 12 | 8 | 35 | N/A | 2998 ± 471 |
| POC-16 | DMO, PAT, TDO, EPSPS | 8 | 6 | N/A | 9 | 12 | No | N/A | 3139 ± 975 |
| | | | | | | | data | | |
| POC-17 | EPSPS, DMO, TDO, PAT | 8 | 3 | N/A | 8 | 3 | 17 | N/A | 3347 ± 589 |
| POC-23 | EPSPS, PAT, TDO, DMO | 3 | 3 | N/A | 12 | 2 | 20 | N/A | 2897 ± 310 |
| POC-24 | EPSPS, DMO, TDO, PAT | No | No | N/A | No | No | No | N/A | 2339 ± 127 |
| | | data | data | | data | data | data | | |
| POC-25 | EPSPS, DMO, TDO, PAT | No | No | N/A | No | No | No | N/A | 2428 ± 426 |
| | | data | data | | data | data | data | | |
| POC-26 | EPSPS, DMO, TDO, PAT, | 8 | 6 | 11 | 6 | 12 | No | No | 1990 ± 529 |
| | PPO_H_N10 | | | | | | data | data | |
| POC-27 | EPSPS, DMO, TDO, PAT, | No | No | No | No | No | No | No | 2244 ± 420 |
| | PPO_H_N90 | data | data | data | data | data | data | data | |
| POC-28 | EPSPS, DMO, TDO, PAT, | 2 | 4 | 2 | 8 | 2 | 17 | 9 | 3438 ± 819 |
| | PPO_H_N90 | | | | | | | | |
| POC-29 | EPSPS, DMO, TDO, PAT, | 7 | 1 | 2 | 1 | 11 | 12 | 12 | 3457 ± 765 |
| | PPO_H_N90 | | | | | | | | |
| POC-32 | PPO_H_N90, EPSPS, | 6 | 2 | 4 | 3 | 8 | No | No | 2210 ± 364 |
| | DMO, TDO, PAT | | | | | | data | data | |
| POC-33 | PPO_H_N90, EPSPS, | 6 | 4 | 1 | 3 | 7 | No | No | 2208 ± 545 |
| | DMO, TDO, PAT | | | | | | data | data | |
| POC-35 | EPSPS, DMO, TDO, PAT, | 3 | 5 | 1 | 1 | 1 | 5 | 4 | 2381 ± 472 |
| | PPO_H_N90 | | | | | | | | |
| Wild-type | N/A | N/A | N/A | N/A | N/A | N/A | N/A | N/A | 2441 ± 364 |

TABLE 8
Protein Expression in the Transgenic Events

| | | # Events | V6 Leaf Protein (ppm) | | | | |
|---|---|---|---|---|---|---|---|
| Construct | Transgene | (Gen) | EPSPS | DMO | PPO | TDO | PAT |
| POC-19 | DMO | 2 (R2) | N/A | 68.86 | N/A | N/A | N/A |
| POC-5 | EPSPS | 1 (R5) | 215.00 | N/A | N/A | N/A | N/A |
| POC-20 | EPSPS | 2 (R4) | 442.84 | N/A | N/A | N/A | N/A |
| POC-2 | TDO | 2 (R4) | N/A | N/A | N/A | 21.53 | N/A |
| POC-4 | PAT | 2 (R3) | N/A | N/A | N/A | N/A | 3.20 |
| POC-21 | PPO_H_N40 | 2 (R2) | N/A | N/A | N/A | N/A | N/A |
| POC-22 | PPO_H_N10 | 6 (R2) | N/A | N/A | 5.68 | N/A | N/A |
| POC-30 | PPO_H_N10, PAT, TDO, | 7 (R2) | 303.57 | 75.83 | 2.41 | 28.50 | 11.57 |
| | DMO, EPSPS | | | | | | |
| POC-31 | PPO_H_N10, PAT, TDO, | 5 (R2) | 368.84 | 85.26 | 1.42 | 35.13 | 10.07 |
| | DMO, EPSPS | | | | | | |
| POC-34 | PPO_H_N90 | 3 (R2) | N/A | N/A | 27.67 | N/A | N/A |
| POC-7 | EPSPS, PAT, TDO, DMO | 7 (R3) | 76.59 | 97.25 | N/A | 34.95 | 11.46 |
| POC-9 | EPSPS, PAT, TDO, DMO | 6 (R3) | 152.76 | 132.29 | N/A | 54.07 | 7.01 |
| POC-14 | EPSPS, PAT, TDO, DMO | 6 (R3) | 81.95 | 20.57 | N/A | 30.31 | 10.4 |
| POC-16 | DMO, PAT, TDO, EPSPS | 6 (R3) | 315.11 | 12.21 | N/A | 47.67 | 6.11 |
| POC-17 | EPSPS, DMO, TDO, PAT | 6 (R3) | 309.03 | 65.75 | N/A | 27.53 | 22 |
| POC-23 | EPSPS, PAT, TDO, DMO | 5 (R2) | 75.42 | 29.95 | N/A | 20.11 | 29.7 |
| POC-24 | EPSPS, DMO, TDO, PAT | 5 (R3) | 323.32 | 30.39 | N/A | 33.01 | 67.01 |
| POC-25 | EPSPS, DMO, TDO, PAT | 6 (R2) | 42.25 | 1.27 | N/A | 55.18 | 10.00 |
| POC-26 | EPSPS, DMO, TDO, PAT, | 5 (R2) | 394.57 | 133.57 | 5.80 | 41.09 | 12.82 |
| | PPO_H_N10 | | | | | | |
| POC-27 | EPSPS, DMO, TDO, PAT, | 2 (R2) | 287.60 | 72.00 | 11.53 | 47.60 | 6.98 |
| | PPO_H_N90 | | | | | | |
| POC-28 | EPSPS, DMO, TDO, PAT, | 2 (R2) | 375.67 | 101.56 | 11.43 | 30.06 | 10.46 |
| | PPO_H_N90 | | | | | | |
| POC-29 | EPSPS, DMO, TDO, PAT, | 7 (R2) | 324.07 | 139.92 | 16.78 | 23.75 | 10.93 |
| | PPO_H_N90 | | | | | | |
| POC-32 | PPO_H_N90, EPSPS, DMO, | 6 (R2) | 550.92 | 42.23 | 9.93 | 59.06 | 18.08 |
| | TDO, PAT | | | | | | |
| POC-33 | PPO_H_N90, EPSPS, DMO, | 6 (R2) | 276.00 | 42.23 | 9.15 | 63.13 | 19.87 |
| | TDO, PAT | | | | | | |
| POC-35 | EPSPS, DMO, TDO, PAT, | 5 (R2) | 298.45 | 217.28 | 5.00 | 29.51 | 22.02 |
| | PPO_H_N90 | | | | | | |

| | |
|---|---|
| Wild-type | undetectable |

### Third-Year Field Trials

The third-year field trials were conducted on homozygous events from POC constructs and commercial constructs. Both efficacy and agronomic trials used a randomized complete block design (RCBD). In the agronomy trials, events were tested at 20 locations across the cotton growing testing region, with 3 replications per location. In the efficacy trials, the events were tested at 3-8 locations with 3 replications per location. Events were evaluated for injury after application of glyphosate, glufosinate, dicamba, mesotrione or tembotrione as the ß-triketone HPPD inhibitor, and flumioxazin and/or epyrifenacil as the PPO herbicide, individually or as a combination of all five chemistries for the five-cassette constructs or four chemistries for the four-cassette constructs (EPSPS, PAT, DMO and TDO). The herbicide treatments are summarized in Table 9). Herbicides were applied at three different growth stages: at pre-planting (PRE) or just prior to planting, at about the 4^{th} leaf stage (V3), and at 10 days after 1^{st} bloom (R2) using a tractor-mounted or backpack sprayer calibrated to deliver 15 gallons per acre (GPA) with water as the herbicide carrier using Teejet Air Induction (TTJ) nozzles. Herbicide injury was evaluated by visual inspection at 7 and 14 days after herbicide applications on a scale of 0 to 100 with “0” being no visible crop injury and “100” being complete crop injury or death.

TABLE 9
Herbicide Treatments in the Third-Year Efficacy Field Trials

| | | | Rate | | | |
|---|---|---|---|---|---|---|
| | | | Pound/ | | Gram/ | Application |
| Treatment | Product Name | Herbicide | Acre | | Hectare | Stage |
| 1 | NA | Untreated | NA | | NA | NA |
| 2 | Roundup Powermax | Glyphosate (Gly) | 2.25 | | 2522 | V3, R2 |
| 3 | Total | Glufosinate (Glu) | 1.06 | | 1190 | V3, R2 |
| 4 | XtendiMax | Dicamba (Dic) | 1 | | 1121 | PRE, V3, R2 |
| | Intact | Drift Reduction Agent | 0.5% | v/v | | PRE, V3, R2 |
| | | (DRA) | | | | |
| | Crop Oil Concentrates | surfactant | 0.375% | v/v | | V3, R2 |
| | (COC) | | | | | |
| 5 | Callisto | Mesotrione (Meso) | 0.4 | | 420 | PRE |
| | Callisto | Mesotrione | 0.2 | | 210 | V3, R2 |
| | COC | surfactant | 0.375% | v/v | | V3, R2 |
| 6 | Laudis | Tembotrione (Tem) | 0.16 | | 184 | V3 |
| | COC | surfactant | 0.375% | v/v | | V3, R2 |
| | Laudis | Tembotrione | 0.08 | | 92 | R2 |
| 7 | Callisto | Mesotrione | 0.4 | | 420 | PRE |
| | XtendiMax | Dicamba | 1 | | 1121 | PRE, V3, R2 |
| | Intact | DRA | 0.5% | v/v | | PRE, V3, R2 |
| | Roundup Powermax | Glyphosate | 2.25 | | 2522 | V3, R2 |
| | Total | Glufosinate | 1.06 | | 1190 | V3, R2 |
| | Callisto | Mesotrione | 0.2 | | 210 | V3, R2 |
| 8 | Laudis | Tembotrione | 0.16 | | 184 | PRE, V3 |
| | XtendiMax | Dicamba | 1 | | 1121 | PRE, V3, R2 |
| | Intact | DRA | 0.5% | v/v | | PRE, V3, R2 |
| | Roundup Powermax | Glyphosate | 2.25 | | 2522 | V3, R2 |
| | Total | Glufosinate | 1.06 | | 1190 | V3, R2 |
| | Laudis | Tembotrione | 0.08 | | 92 | R2 |
| 9 | Valor SX | Flumioxazin (Flu) | 0.188 | | 210 | PRE |
| | Rapidicil | Epyrifenacil (Epy) | 0.036 | | 40 | V3, R2 |
| | COC | surfactant | 0.375% | v/v | | V3, R2 |
| 10 | Valor SX | Flumioxazin | 0.375 | | 420 | PRE |
| | Rapidicil | Epyrifenacil | 0.072 | | 80 | V3, R2 |
| | COC | surfactant | 0.375% | v/v | | V3, R2 |
| 11 | Valor SX | Flumioxazin | 0.188 | | 210 | PRE, V3 |
| | COC | surfactant | 0.375% | v/v | | V3, R2 |
| | Valor SX | Flumioxazin | 0.094 | | 105 | R2 |
| 12 | Callisto | Mesotrione | 0.4 | | 420 | PRE |
| | Valor SX | Flumioxazin | 0.188 | | 210 | PRE |
| | XtendiMax | Dicamba | 1 | | 1121 | PRE, V3, R2 |
| | Intact | DRA | 0.5% | v/v | | PRE, V3, R2 |
| | Roundup Powermax | Glyphosate | 2.25 | | 2522 | V3, R2 |
| | Total | Glufosinate | 1.06 | | 1190 | V3, R2 |
| | Callisto | Mesotrione | 0.2 | | 210 | V3, R2 |
| | Rapidicil | Epyrifenacil | 0.036 | | 40 | V3, R2 |
| 13 | Callisto | Mesotrione | 0.4 | | 420 | PRE |
| | XtendiMax | Dicamba | 1 | | 1121 | PRE, V3, R2 |
| | Intact | DRA | 0.5% | v/v | | PRE, V3, R2 |

| | | | | |
|---|---|---|---|---|
| Roundup Powermax | Glyphosate | 2.25 | 2522 | V3, R2 |
| Total | Glufosinate | 1.06 | 1190 | V3, R2 |
| Callisto | Mesotrione | 0.2 | 210 | V3, R2 |
| Rapidicil | Epyrifenacil | 0.072 | 80 | V3, R2 |

Plots were two rows by forty feet long with a ten-foot alley with 30-inch row spacing and seeded at 235 seeds per plot. Trial maintenance was designed to optimize cotton yield. Data were collected for stand count, boll open percent, yield, and fiber quality characteristics. Leaf samples were also collected at V6 stage for measurement of protein levels by ELISA.

All data from both agronomic and efficacy trials were subject to analysis of variance and means separated at LSD at alpha 0.05. Statistical analysis was automated using ASReml software for mixed model fitting. Constructs/events were compared to the wild-type transformation germplasm for any significant agronomic differences, including yield, flowering time and fiber traits for meeting advancement criteria of no off-type phenotypes, no significant difference in yield (less than 10%) and fiber quality compared the wild-type germplasm, and no greater than 10% injury to single herbicide treatment or 20% injury to tank mix herbicides when applied at 2× commercial rates. Yield was taken by a standard picker on a 40 ft planted plot and calculated out to the acre.

The results of the third-year agronomics trials are shown in Table 10. In the table headings, “# Trangene Cassette refers to the number of herbicide tolerance transgenic cassettes. “Triple” refers to plants containing the BAR (encoding the phosphinothricin N-acetyltransferase enzyme from Streptomyces hygroscopicus for conferring tolerance to glufosinate), DMO and EPSPS cassettes; “Quad” refers to events containing the PAT, DMO, EPSPS and TDO cassettes. “Penta” refers to events containing the PAT, DMO, EPSPS, TDO and PPO cassettes. “Penta (AE)” refers to events containing the additional Cre cassette for excision of the selection cassette aadA for production of marker free events. Broad Acre Yield (BAY) was measured as pound per acre of total seed cotton. “WT Control” stands for wild-type control. As shown in Table 10, majority of the events showed similar yield when compared to the WT control. Events showing 10% or greater yield reduction (shown in bold font) were not advanced for further testing. Event GhHT4-1-18 (shown in bold and italic font) was later selected as the commercial event based on molecular characteristics and field performance among many tested events, and named Gh_CSM63718.

TABLE 10
Summary of Yield Results in the
Third-Year Agronomic Field Trials

| | # Transgene | | BAY |
|---|---|---|---|
| Construct | Cassette | Event | (lb/acre) |
| WT Control | — | WT Control | 3338 |
| Commercial Check | Triple | Commercial Check | 3470 |
| GhHT4-1 | PENTA (AE) | GhHT4-1-1 | 3452 |
| | | GhHT4-1-2 | 3216 |
| | | GhHT4-1-3 | 3449 |
| | | GhHT4-1-4 | 3365 |
| | | GhHT4-1-5 | 3414 |
| | | GhHT4-1-6 | 3592 |
| | | GhHT4-1-7 | 3551 |
| | | GhHT4-1-8 | 3347 |
| | | GhHT4-1-9 | 3348 |
| | | GhHT4-1-10 | 3402 |
| | | GhHT4-1-11 | 3642 |
| | | GhHT4-1-12 | 3581 |
| | | GhHT4-1-13 | 3530 |
| | | GhHT4-1-14 | 3433 |
| | | GhHT4-1-15 | 3528 |
| | | GhHT4-1-16 | 3271 |
| | | GhHT4-1-17 | 3387 |
| | | GhHT4-1-18 | 3356 |
| | | GhHT4-1-19 | 3456 |
| | | GhHT4-1-20 | 3394 |
| | | GhHT4-1-21 | 3311 |
| | | GhHT4-1-22 | 3528 |
| | | GhHT4-1-23 | 3404 |
| | | GhHT4-1-24 | 3343 |
| GhHT4-2 | PENTA | GhHT4-2-1 | 3412 |
| | | GhHT4-2-2 | 3434 |
| | | GhHT4-2-3 | 3419 |
| | | GhHT4-2-4 | 3467 |
| | | GhHT4-2-5 | 3399 |
| | | GhHT4-2-6 | 3359 |
| | | GhHT4-2-7 | 3400 |
| | | GhHT4-2-8 | 2992 |
| | | GhHT4-2-9 | 3487 |
| | | GhHT4-2-10 | 3536 |
| | | GhHT4-2-11 | 3356 |
| | | GhHT4-2-12 | 3181 |
| | | GhHT4-2-13 | 3371 |
| | | GhHT4-2-14 | 3347 |
| | | GhHT4-2-15 | 3613 |
| | | GhHT4-2-16 | 3264 |
| | | GhHT4-2-17 | 3204 |
| | | GhHT4-2-18 | 3346 |
| | | GhHT4-2-19 | 3417 |
| | | GhHT4-2-20 | 3444 |
| GhHT4-3 | PENTA | GhHT4-3-1 | 3345 |
| | | GhHT4-3-2 | 3429 |
| | | GhHT4-3-3 | 3360 |
| | | GhHT4-3-4 | 3452 |
| | | GhHT4-3-5 | 3097 |
| | | GhHT4-3-6 | 3295 |
| | | GhHT4-3-7 | 3385 |
| | | GhHT4-3-8 | 3204 |
| | | GhHT4-3-9 | 3285 |
| | | GhHT4-3-10 | 3163 |
| | | GhHT4-3-11 | 3261 |
| | | GhHT4-3-12 | 3308 |
| | | GhHT4-3-13 | 3306 |
| | | GhHT4-3-14 | 3317 |
| | | GhHT4-3-15 | 3407 |
| | | GhHT4-3-16 | 2972 |
| | | GhHT4-3-17 | 3159 |
| | | GhHT4-3-18 | 3255 |
| | | GhHT4-3-19 | 3340 |
| | | GhHT4-3-20 | 3246 |
| | | GhHT4-3-21 | 3312 |
| | | GhHT4-3-22 | 3183 |
| | | GhHT4-3-23 | 3426 |
| | | GhHT4-3-24 | 3396 |
| | | GhHT4-3-25 | 3312 |
| GhHT4-4 | QUAD | GhHT4-4-1 | 3379 |
| | | GhHT4-4-2 | 3500 |
| | | GhHT4-4-3 | 3422 |
| | | GhHT4-4-4 | 3416 |
| | | GhHT4-4-5 | 3258 |
| | | GhHT4-4-6 | 3495 |
| | | GhHT4-4-7 | 3500 |
| | | GhHT4-4-8 | 3392 |
| | | GhHT4-4-9 | 3516 |
| | | GhHT4-4-10 | 3503 |
| | | GhHT4-4-11 | 3365 |
| | | GhHT4-4-12 | 3376 |
| | | GhHT4-4-13 | 3452 |
| | | GhHT4-4-14 | 3399 |
| | | GhHT4-4-15 | 3347 |
| | | GhHT4-4-16 | 3553 |
| | | GhHT4-4-17 | 3351 |
| | | GhHT4-4-18 | 3434 |
| | | GhHT4-4-19 | 3533 |
| | | GhHT4-4-20 | 3468 |
| POC-17 | QUAD | POC-17-1 | 3472 |
| | | POC-17-2 | 3344 |
| POC-32 | PENTA (AE) | POC-32-1 | 3557 |
| | | POC-32-2 | 3598 |
| | | POC-32-3 | 3330 |
| | | POC-32-4 | 3452 |
| POC-35 | PENTA | POC-35-1 | 3544 |
| | | POC-35-2 | 3279 |
| POC-36 | QUAD | POC-36-1 | 3417 |
| | | POC-36-2 | 3368 |
| | | POC-36-3 | 3146 |
| | | POC-36-4 | 3323 |
| | | POC-36-5 | 3498 |
| | | POC-36-6 | 3070 |
| | | POC-36-7 | 3374 |
| | | POC-36-8 | 3267 |
| POC-37 | PENTA | POC-37-1 | 3260 |
| | | POC-37-2 | 3608 |
| | | POC-37-3 | 3762 |
| | | POC-37-4 | 3390 |
| | | POC-37-5 | 3391 |
| | | POC-37-6 | 3465 |
| | | POC-37-7 | 3490 |
| | | POC-37-8 | 3179 |
| POC-38 | QUAD | POC-38-1 | 3418 |
| | | POC-38-2 | 3241 |
| | | POC-38-3 | 3419 |
| | | POC-38-4 | 3145 |
| | | POC-38-5 | 3437 |
| | | POC-38-6 | 3172 |
| | | POC-38-7 | 3031 |
| | | POC-38-8 | 3050 |
| POC-39 | PENTA | POC-39-1 | 3630 |
| | | POC-39-2 | 3563 |
| POC-40 | PENTA (AE) | POC-40-1 | 3500 |
| | | POC-40-2 | 3523 |
| | | POC-40-3 | 3558 |
| | | POC-40-4 | 3676 |
| POC-43 | QUAD | POC-43-1 | 3334 |
| | | POC-43-2 | 3329 |
| | | POC-43-3 | 2940 |
| | | POC-43-4 | 2693 |
| | | POC-43-5 | 3033 |
| | | POC-43-6 | 3155 |

In the efficacy trials, events were bulked by construct due to low seed availability. Briefly, no significant crop injury was observed when the herbicides were applied at the PRE planting stage (data not shown). Some injuries were observed at the construct level for the post planting application of tank mix of four or five different herbicides at 7 days after the herbicide application (data not shown). Since the injury was only limited to the tank mix at one time point and at construct level, further testing was conducted.

The third-year efficacy trials also included a relatively low powered trial due to seed availability to evaluate performance of individual events of the three penta-cassette commercial transformation constructs, which consisted of one location with three replications in a 5-foot planted plot. The results of the trial are shown in Table 11. All but one event (GhHT4-2-10) performed well after application of single herbicides. Most events showed some injuries after the tank mix application. Several events show ≥20% injury (shown in bold font). These events were not advanced for further testing. In Table 11, “Gly” refers to glyphosate, “Die” refers to dicamba, “Epy” refers to epyrifenacil, “Meso” refers to mesotrione, and “Glu” refers to glufosinate.

Based on the efficacy and agronomic trial data and molecular characterization of the events such as insertion location in the genome, events from the three penta-cassette constructs (GhHT4-1, GhHT4-2 and GhHT4-3) were selected for advancement for further field trials.

TABLE 11
Performance of Events in the Third-Year Efficacy Field Trials

| Event | Herbicide Injury Rating (%) | | | | | | |
|---|---|---|---|---|---|---|---|
| Name | 2x Gly | 2x Dic | 2x Epy | 4x Epy | 2x Mes | 2x Glu | 2x 4-Way Tank Mix |
| GhHT4-1-5 | 2 | 0 | 0 | 0 | 0 | 0 | 12 |
| GhHT4-1-6 | 0 | 2 | 0 | 0 | 10 | 0 | 23 |
| GhHT4-1-7 | 3 | 0 | 0 | 0 | 0 | 0 | 17 |
| GhHT4-1-8 | 0 | 2 | 0 | 0 | 0 | 2 | 18 |
| GhHT4-1-9 | 0 | 2 | 0 | 0 | 0 | 0 | 0 |
| GhHT4-1-10 | 0 | 0 | 0 | 0 | 0 | 0 | 13 |
| GhHT4-1-11 | 0 | 0 | 2 | 0 | 0 | 0 | 12 |
| GhHT4-1-12 | 0 | 8 | 0 | 0 | 0 | 0 | 14 |
| GhHT4-1-13 | 0 | 7 | 0 | 0 | 2 | 0 | 18 |
| GhHT4-1-14 | 0 | 3 | 0 | 0 | 0 | 0 | 15 |
| GhHT4-1-15 | 4 | 2 | 0 | 0 | 5 | 0 | 19 |
| GhHT4-1-16 | 0 | 4 | 2 | 0 | 0 | 0 | 20 |
| GhHT4-1-17 | 0 | 0 | 0 | 2 | 0 | 0 | 18 |
| GhHT4-1-18 | 2 | 0 | 0 | 2 | 2 | 0 | 19 |
| GhHT4-1-19 | 0 | 3 | 0 | 0 | 0 | 2 | 19 |
| GhHT4-1-20 | 0 | 2 | 0 | 0 | 2 | 2 | 20 |
| GhHT4-1-21 | 0 | 0 | 0 | 0 | 0 | 0 | 23 |
| GhHT4-1-22 | 0 | 0 | 0 | 4 | 0 | 2 | 18 |
| GhHT4-1-23 | 0 | 2 | 0 | 0 | 0 | 0 | 15 |
| GhHT4-1-24 | 0 | 0 | 0 | 8 | 0 | 0 | 23 |
| GhHT4-2-1 | 2 | 0 | 0 | 0 | 0 | 0 | 11 |
| GhHT4-2-2 | 0 | 3 | 0 | 0 | 0 | 2 | 0 |
| GhHT4-2-3 | 0 | 6 | 0 | 0 | 2 | 2 | 14 |
| GhHT4-2-4 | 0 | 3 | 0 | 0 | 0 | 0 | 18 |
| GhHT4-2-5 | 0 | 0 | 0 | 2 | 0 | 0 | 22 |
| GhHT4-2-6 | 0 | 0 | 2 | 0 | 0 | 0 | 25 |
| GhHT4-2-7 | 0 | 0 | 0 | 0 | 0 | 4 | 18 |
| GhHT4-2-8 | 0 | 0 | 0 | 0 | 0 | 0 | 9 |
| GhHT4-2-9 | 0 | 4 | 0 | 0 | 0 | 2 | 19 |
| GhHT4-2-10 | 28 | 0 | 70 | 0 | 0 | 80 | 80 |
| GhHT4-2-11 | 0 | 0 | 0 | 0 | 2 | 0 | 20 |
| GhHT4-2-12 | 0 | 0 | 0 | 0 | 3 | 0 | 12 |
| GhHT4-2-13 | 2 | 0 | 0 | 0 | 0 | 0 | 12 |
| GhHT4-2-14 | 0 | 0 | 0 | 0 | 0 | 2 | 23 |
| GhHT4-2-15 | 0 | 0 | 0 | 2 | 0 | 0 | 10 |
| GhHT4-2-16 | 0 | 0 | 0 | 2 | 0 | 0 | 19 |
| GhHT4-2-17 | 0 | 0 | 0 | 0 | 0 | 0 | 20 |
| GhHT4-2-18 | 2 | 0 | 0 | 0 | 0 | 2 | 13 |
| GhHT4-2-19 | 0 | 3 | 0 | 0 | 0 | 3 | 0 |
| GhHT4-2-20 | 0 | 0 | 0 | 0 | 0 | 0 | 16 |
| GhHT4-3-6 | 0 | 0 | 0 | 0 | 2 | 3 | 19 |
| GhHT4-3-7 | 0 | 0 | 0 | 0 | 0 | 2 | 19 |
| GhHT4-3-8 | 0 | 2 | 0 | 0 | 0 | 0 | 22 |
| GhHT4-3-9 | 0 | 0 | 0 | 0 | 0 | 0 | 15 |
| GhHT4-3-10 | 0 | 2 | 0 | 0 | 0 | 0 | 16 |
| GhHT4-3-11 | 0 | 0 | 0 | 0 | 0 | 0 | 14 |
| GhHT4-3-12 | 2 | 0 | 0 | 0 | 0 | 2 | 18 |
| GhHT4-3-13 | 0 | 0 | 0 | 2 | 0 | 0 | 19 |
| GhHT4-3-14 | 0 | 0 | 0 | 0 | 0 | 0 | 23 |
| GhHT4-3-15 | 3 | 2 | 0 | 0 | 0 | 0 | 18 |
| GhHT4-3-16 | 0 | 3 | 0 | 0 | 0 | 0 | 16 |
| GhHT4-3-17 | 0 | 2 | 0 | 2 | 0 | 0 | 14 |
| GhHT4-3-18 | 0 | 0 | 2 | 0 | 0 | 0 | 23 |
| GhHT4-3-19 | 0 | 0 | 0 | 0 | 0 | 0 | 20 |
| GhHT4-3-20 | 0 | 0 | 0 | 0 | 0 | 0 | 19 |
| GhHT4-3-21 | 3 | 0 | 0 | 0 | 0 | 3 | 0 |
| GhHT4-3-22 | 0 | 2 | 3 | 2 | 0 | 0 | 17 |
| GhHT4-3-23 | 0 | 2 | 2 | 0 | 0 | 2 | 23 |
| GhHT4-3-24 | 0 | 0 | 0 | 0 | 4 | 0 | 23 |
| GhHT4-3-25 | 0 | 0 | 2 | 0 | 0 | 0 | 13 |

### Fourth-Year Field Trials

In the fourth-year field agronomic trials, thirty events from the penta-cassette constructs were tested at 20 locations across the cotton growing region, with 3 replications per location using a RCBD design. Seeds were planted in 2 rows, 40 ft long plots and evaluated for stand count, boll open percent, yield and fiber traits. The trials also included a wild-type control and a commercial check.

In the efficacy trials, these same events were tested at 10 locations with 3 replications per location. Herbicide treatments included glufosinate, glyphosate, dicamba, mesotrione and epyrifenacil individually, applied at 2× commercial rates at V3 (3^{rd} node) and R2 (early bloom) stages. Herbicide injury was evaluated by visual inspection at 3, 7 and 14 days after herbicide applications on a scale of 0 to 100 with “0” being no visible crop injury and “100” being complete crop injury or death.

All data from both agronomic and efficacy trials were subject to analysis of variance and means separated at LSD at alpha 0.05. Statistical analysis was automated using ASReml software for mixed model fitting.

Table 12 summarizes the yield results from the fourth-year agronomic field trials. Yield is expressed as pound per acre of total seed cotton. “Delta from WT” is the yield difference between the event and the WT control. “% Delta from WT” was calculated as “Delta from WT”/3248.46 (WT yield)×100. 5% Delta from WT was used as one of the advancement criteria. Events not meeting the advancement criteria are highlighted by bold font in Table 12. The results show that the GhHT4-1 events performed much better than events from the other two constructs, and all events met the yield-based advancement criteria from the agronomic trials.

TABLE 12
Yield Performance of the Events in
the Fourth-Year Agronomic Trials

| | Total Seed Cotton | Delta | % Delta |
|---|---|---|---|
| Event Name | (lb/acre) | from WT | from WT |
| WT Control | 3248.46 | — | — |
| Commercial check | 3372.41 | 123.95 | 3.7 |
| GhHT4-1-7 | 3309.37 | 60.91 | 1.8 |
| GhHT4-1-10 | 3314.26 | 65.80 | 2.0 |
| GhHT4-1-11 | 3336.83 | 88.37 | 2.6 |
| GhHT4-1-13 | 3351.64 | 103.18 | 3.1 |
| GhHT4-1-16 | 3258.71 | 10.25 | 0.3 |
| GhHT4-1-17 | 3295.47 | 47.01 | 1.4 |
| GhHT4-1-18 | 3307.43 | 58.97 | 1.8 |
| GhHT4-1-20 | 3394.23 | 145.77 | 4.3 |
| GhHT4-1-21 | 3319.58 | 71.12 | 2.1 |
| GhHT4-1-22 | 3410.97 | 162.51 | 4.8 |
| GhHT4-1-23 | 3322.64 | 74.18 | 2.2 |
| GhHT4-1-24 | 3288.88 | 40.42 | 1.2 |
| GhHT4-2-1 | 3014.14 | −234.32 | −7.8 |
| GhHT4-2-3 | 2919.02 | −329.44 | −11.3 |
| GhHT4-2-4 | 3104.58 | −143.88 | −4.6 |
| GhHT4-2-6 | 3024.20 | −224.26 | −7.4 |
| GhHT4-2-7 | 2979.83 | −268.63 | −9.0 |
| GhHT4-2-9 | 3136.56 | −111.90 | −3.6 |
| GhHT4-2-11 | 3165.90 | −82.56 | −2.6 |
| GhHT4-2-12 | 3083.13 | −165.33 | −5.4 |
| GhHT4-2-13 | 3077.53 | −170.93 | −5.6 |
| GhHT4-2-14 | 3063.47 | −184.99 | −6.0 |
| GhHT4-2-15 | 3147.92 | −100.54 | −3.2 |
| GhHT4-2-18 | 3075.20 | −173.26 | −5.6 |
| GhHT4-3-6 | 3159.64 | −88.82 | −2.8 |
| GhHT4-3-8 | 2958.10 | −290.36 | −9.8 |
| GhHT4-3-11 | 3065.15 | −183.31 | −6.0 |
| GhHT4-3-12 | 3548.09 | 175.68 | 8.4 |
| GhHT4-3-23 | 3076.45 | −172.01 | −5.6 |
| GhHT4-3-25 | 3091.32 | −157.14 | −5.1 |

The efficacy trials included five single herbicide treatments as described in the preceding paragraph. Table 13 shows the across-location analysis of herbicide injury in the efficacy trial results. Events from all three constructs tested performed well. Only one event (GhHT4-1-20) showed more than 10% injury over all three applications of the PPO herbicide. In addition to assessing the performance of the events to single herbicide treatments at 2× commercial rates, the events were also evaluated for performance to 4× commercial rates of glufosinate, glyphosate, mesotrione and epyrifenacil, both individually and in a tank mix of glufosinate, glyphosate, dicamba, mesotrione and epyrifenacil, in a one location efficacy trial (Table 14). As shown in Table 13 and Table 14, construct GhHT4-1 consistently performed better, though not statistically, than the other two constructs.

TABLE 13
Summary of the Fourth-Year Efficacy Trials Results

| | 2 × | | | 2 × | | | 2 × | | | 2 × | | | 2 × | | |
|---|---|---|---|---|---|---|---|---|---|---|---|---|---|---|---|
| | Glyphosate | | | glufosinate | | | Dicamba | | | Mesotrione | | | Epyrifenacil | | |
| Event | Injury Rating (Days after Treatment) | | | | | | | | | | | | | | |
| Name | 3 | 7 | 14 | 3 | 7 | 14 | 3 | 7 | 14 | 3 | 7 | 14 | 3 | 7 | 14 |
| Commercial | 6 | 2 | 0 | 10 | 2 | 0 | 4 | 2 | 0 | N/A | N/A | N/A | N/A | N/A | N/A |
| Check | | | | | | | | | | | | | | | |
| GhHT4-1-7 | 3 | 2 | 0 | 6 | 1 | 1 | 4 | 1 | 0 | 2 | 1 | 1 | 1 | 0 | 0 |
| GhHT4-1-10 | 2 | 2 | 0 | 5 | 2 | 2 | 4 | 1 | 1 | 2 | 1 | 0 | 1 | 1 | 0 |
| GhHT4-1-11 | 2 | 2 | 0 | 6 | 2 | 3 | 4 | 1 | 1 | 2 | 1 | 2 | 2 | 1 | 0 |
| GhHT4-1-13 | 2 | 2 | 0 | 8 | 2 | 1 | 4 | 1 | 0 | 2 | 1 | 3 | 2 | 1 | 1 |
| GhHT4-1-16 | 2 | 2 | 0 | 6 | 1 | 0 | 4 | 1 | 1 | 2 | 1 | 1 | 2 | 1 | 0 |
| GhHT4-1-17 | 2 | 2 | 0 | 6 | 2 | 1 | 3 | 1 | 1 | 2 | 1 | 1 | 2 | 0 | 0 |
| GhHT4-1-18 | 2 | 2 | 0 | 6 | 2 | 2 | 4 | 1 | 0 | 1 | 1 | 3 | 2 | 1 | 0 |
| GhHT4-1-20 | 2 | 2 | 0 | 7 | 2 | 2 | 4 | 1 | 0 | 1 | 1 | 0 | 2 | 1 | 11 |
| GhHT4-1-21 | 2 | 2 | 0 | 8 | 2 | 1 | 4 | 2 | 0 | 2 | 4 | 2 | 1 | 1 | 0 |
| GhHT4-1-22 | 2 | 2 | 0 | 6 | 2 | 1 | 4 | 1 | 0 | 1 | 1 | 1 | 1 | 1 | 0 |
| GhHT4-1-23 | 2 | 2 | 0 | 6 | 2 | 1 | 4 | 2 | 1 | 1 | 1 | 2 | 2 | 1 | 0 |
| GhHT4-1-24 | 3 | 2 | 0 | 6 | 2 | 0 | 4 | 1 | 0 | 1 | 1 | 2 | 2 | 1 | 0 |
| GhHT4-2-1 | 2 | 2 | 1 | 8 | 2 | 1 | 4 | 1 | 0 | 1 | 1 | 0 | 2 | 1 | 0 |
| GhHT4-2-3 | 2 | 2 | 0 | 7 | 2 | 2 | 4 | 1 | 1 | 2 | 1 | 0 | 1 | 1 | 0 |
| GhHT4-2-4 | 2 | 2 | 0 | 6 | 2 | 0 | 4 | 1 | 0 | 2 | 4 | 2 | 2 | 1 | 0 |
| GhHT4-2-6 | 2 | 2 | 0 | 5 | 2 | 0 | 4 | 1 | 0 | 2 | 1 | 0 | 2 | 1 | 0 |
| GhHT4-2-7 | 2 | 3 | 0 | 8 | 2 | 3 | 4 | 1 | 1 | 2 | 1 | 0 | 3 | 1 | 1 |
| GhHT4-2-9 | 2 | 2 | 0 | 5 | 2 | 1 | 4 | 1 | 0 | 2 | 3 | 0 | 2 | 1 | 0 |
| GhHT4-2-11 | 2 | 2 | 0 | 5 | 2 | 2 | 4 | 2 | 0 | 2 | 1 | 2 | 2 | 1 | 0 |
| GhHT4-2-12 | 2 | 2 | 0 | 6 | 2 | 0 | 3 | 1 | 0 | 1 | 1 | 0 | 2 | 1 | 0 |
| GhHT4-2-13 | 2 | 2 | 0 | 8 | 2 | 1 | 4 | 1 | 0 | 2 | 1 | 3 | 2 | 1 | 0 |
| GhHT4-2-14 | 4 | 2 | 0 | 7 | 2 | 1 | 3 | 1 | 1 | 1 | 1 | 2 | 2 | 1 | 0 |
| GhHT4-2-15 | 2 | 2 | 0 | 6 | 2 | 1 | 4 | 1 | 1 | 1 | 1 | 1 | 1 | 1 | 0 |
| GhHT4-2-18 | 2 | 2 | 0 | 6 | 2 | 1 | 4 | 1 | 1 | 2 | 5 | 0 | 2 | 1 | 0 |
| GhHT4-3-6 | 2 | 2 | 0 | 6 | 2 | 2 | 4 | 1 | 0 | 1 | 1 | 1 | 2 | 1 | 0 |
| GhHT4-3-8 | 2 | 2 | 1 | 6 | 2 | 1 | 4 | 1 | 1 | 2 | 1 | 0 | 1 | 1 | 0 |
| GhHT4-3-11 | 2 | 2 | 0 | 6 | 1 | 0 | 3 | 2 | 1 | 2 | 1 | 0 | 1 | 0 | 1 |
| GhHT4-3-12 | 2 | 2 | 0 | 7 | 2 | 1 | 4 | 1 | 0 | 2 | 1 | 1 | 2 | 1 | 0 |
| GhHT4-3-23 | 3 | 2 | 0 | 7 | 2 | 0 | 3 | 1 | 0 | 2 | 1 | 2 | 2 | 0 | 0 |
| GhHT4-3-25 | 3 | 2 | 0 | 7 | 2 | 0 | 4 | 1 | 1 | 1 | 1 | 2 | 2 | 1 | 0 |

TABLE 14
Summary of Crop Injury at Higher Concentrations of Single Herbicides and Tank Mix

| | Untreated | | | 4 × | | | 4 × | | | 4 × | | | 4 × | | | 4 × 5 Way | | |
|---|---|---|---|---|---|---|---|---|---|---|---|---|---|---|---|---|---|---|
| | Control | | | Glyphosate | | | Glufosinate | | | Mesotrione | | | Epyrifenacil | | | Tank Mix | | |
| Event | Injury Rating (Days after Treatment) | | | | | | | | | | | | | | | | | |
| Name | 3 | 7 | 14 | 3 | 7 | 14 | 3 | 7 | 14 | 3 | 7 | 14 | 3 | 7 | 14 | 3 | 7 | 14 |
| Commercial | 9 | 15 | 0 | 23 | 25 | 0 | 23 | 30 | 0 | N/A | N/A | N/A | N/A | N/A | N/A | N/A | N/A | N/A |
| Check | | | | | | | | | | | | | | | | | | |
| GhHT4-1-7 | 8 | 15 | 0 | 15 | 22 | 0 | 17 | 27 | 0 | 15 | 20 | 3 | 35 | 18 | 0 | 20 | 23 | 3 |
| GhHT4-1-10 | 8 | 17 | 0 | 12 | 25 | 2 | 12 | 30 | 2 | 10 | 23 | 1 | 10 | 17 | 2 | 18 | 27 | 5 |
| GhHT4-1-11 | 12 | 23 | 0 | 15 | 22 | 0 | 17 | 18 | 2 | 10 | 20 | 34 | 13 | 17 | 4 | 17 | 27 | 3 |
| GhHT4-1-13 | 8 | 8 | 0 | 15 | 23 | 2 | 12 | 17 | 0 | 10 | 15 | 3 | 10 | 18 | 0 | 22 | 38 | 3 |
| GhHT4-1-16 | 11 | 17 | 0 | 15 | 17 | 1 | 18 | 22 | 1 | 13 | 17 | 4 | 15 | 26 | 4 | 15 | 32 | 5 |
| GhHT4-1-17 | 6 | 15 | 0 | 12 | 27 | 0 | 17 | 25 | 2 | 12 | 25 | 3 | 15 | 18 | 0 | 10 | 20 | 2 |
| GhHT4-1-18 | 9 | 17 | 2 | 13 | 20 | 0 | 23 | 18 | 1 | 17 | 22 | 2 | 15 | 20 | 3 | 18 | 25 | 4 |
| GhHT4-1-20 | 7 | 18 | 0 | 12 | 18 | 0 | 10 | 23 | 2 | 18 | 25 | 0 | 10 | 18 | 2 | 17 | 42 | 4 |
| GhHT4-1-21 | 4 | 17 | 0 | 18 | 20 | 1 | 17 | 22 | 1 | 15 | 22 | 0 | 12 | 22 | 0 | 12 | 17 | 1 |
| GhHT4-1-22 | 9 | 18 | 0 | 12 | 20 | 0 | 17 | 23 | 2 | 17 | 22 | 0 | 10 | 20 | 1 | 13 | 25 | 1 |
| GhHT4-1-23 | 7 | 18 | 0 | 13 | 20 | 0 | 17 | 23 | 3 | 15 | 25 | 4 | 12 | 20 | 3 | 20 | 33 | 4 |
| GhHT4-1-24 | 6 | 12 | 0 | 10 | 20 | 3 | 15 | 23 | 0 | 12 | 23 | 2 | 8 | 15 | 3 | 15 | 25 | 3 |
| GhHT4-2-1 | 7 | 23 | 0 | 15 | 25 | 0 | 13 | 20 | 0 | 12 | 42 | 4 | 8 | 18 | 0 | 17 | 30 | 2 |
| GhHT4-2-3 | 8 | 0 | 20 | 18 | 17 | 2 | 15 | 17 | 1 | 10 | 22 | 0 | 8 | 20 | 2 | 13 | 20 | 1 |
| GhHT4-2-4 | 7 | 13 | 0 | 12 | 17 | 0 | 8 | 17 | 0 | 18 | 28 | 0 | 10 | 15 | 0 | 15 | 25 | 3 |
| GhHT4-2-6 | 9 | 18 | 0 | 12 | 25 | 1 | 17 | 27 | 0 | 12 | 30 | 1 | 13 | 22 | 3 | 12 | 23 | 4 |
| GhHT4-2-7 | 14 | 20 | 2 | 22 | 22 | 1 | 15 | 15 | 1 | 13 | 23 | 2 | 13 | 23 | 2 | 22 | 22 | 0 |
| GhHT4-2-9 | 8 | 18 | 0 | 12 | 23 | 0 | 17 | 18 | 0 | 12 | 23 | 2 | 10 | 20 | 3 | 13 | 28 | 0 |
| GhHT4-2-11 | 4 | 18 | 0 | 15 | 23 | 2 | 17 | 28 | 2 | 15 | 23 | 2 | 15 | 15 | 2 | 13 | 30 | 2 |
| GhHT4-2-12 | 11 | 18 | 0 | 17 | 17 | 0 | 12 | 20 | 3 | 18 | 22 | 2 | 10 | 22 | 2 | 10 | 23 | 4 |
| GhHT4-2-13 | 8 | 15 | 0 | 12 | 17 | 0 | 17 | 20 | 2 | 13 | 25 | 3 | 12 | 20 | 0 | 12 | 28 | 53 |
| GhHT4-2-14 | 6 | 17 | 0 | 17 | 22 | 2 | 17 | 20 | 0 | 13 | 22 | 3 | 12 | 18 | 3 | 15 | 35 | 5 |
| GhHT4-2-15 | 9 | 22 | 0 | 1 | 20 | 3 | 17 | 18 | 0 | 18 | 23 | 2 | 13 | 23 | 0 | 13 | 43 | 5 |
| GhHT4-2-18 | 8 | 12 | 0 | 22 | 25 | 0 | 20 | 28 | 2 | 17 | 25 | 3 | 10 | 18 | 3 | 38 | 20 | 3 |
| GhHT4-3-6 | 6 | 20 | 0 | 12 | 15 | 0 | 10 | 23 | 2 | 13 | 28 | 2 | 15 | 15 | 3 | 13 | 30 | 4 |
| GhHT4-3-8 | 8 | 20 | 0 | 15 | 20 | 0 | 27 | 25 | 3 | 13 | 27 | 0 | 20 | 20 | 2 | 15 | 30 | 3 |
| GhHT4-3-11 | 10 | 15 | 2 | 13 | 23 | 0 | 17 | 22 | 0 | 8 | 22 | 4 | 15 | 23 | 2 | 18 | 25 | 3 |
| GhHT4-3-23 | 6 | 18 | 0 | 13 | 15 | 3 | 17 | 27 | 0 | 8 | 22 | 2 | 12 | 20 | 3 | 22 | 22 | 1 |
| GhHT4-3-25 | 9 | 23 | 0 | 15 | 20 | 0 | 20 | 15 | 0 | 10 | 28 | 0 | 12 | 18 | 0 | 27 | 32 | 3 |

### Fifth-Year Field Trials

Based on field trial performance, molecular characteristics, and additional considerations, multiple events from constructs GhHT4-1 and GhHT4-2 were selected and advanced to the fifth-year field trials to evaluate and select the top performing event, along with a wild-type control and a commercial check, which is tolerant to glyphosate, glufosinate and dicamba (Bollgard II® Xtend Flex®).

The events were tested at 20 locations across the cotton growing region, with 3 replications per location using a RCBD design. Seeds were planted in 2 rows, 40 ft long plots and evaluated for stand count, boll open percent, yield and fiber traits.

In the efficacy trials events were tested at 10 locations with 3 replications per location. Herbicide treatments included glufosinate, glyphosate, dicamba, mesotrione, and epyrifenacil individually, applied at 2× commercial rates at V3 (3^{rd} node) stage. Herbicide injury was evaluated by visual inspection at 7 and 14 days after herbicide applications on a scale of 0 to 100 with “0” being no visible crop injury and “100” being complete crop injury or death.

Table 15 shows the across-location analysis of yield in the agronomic trials. All events performed well. No significant difference in yield was observed among all the events. Furthermore, all events demonstrated greater yield than the wild-type control in the trials as shown in the % Delta from WT column, which is calculated as (Event Yield—WT Yield)/WT yield)×100.

TABLE 15
Transgenic Event Yield Performance in the 5^{th}-Year Trials

| | Yield (lb/A of Total | % Delta |
|---|---|---|
| Event Name | Seed Cotton) | from WT |
| WT control | 3246.23 | — |
| Commercial check | 4191.17 | 29.11 |
| GhHT4-1-10 | 3737.66 | 15.14 |
| GhHT4-1-11 | 3606.30 | 11.09 |
| GhHT4-1-13 | 3748.69 | 15.48 |
| GhHT4-1-17 | 3623.15 | 11.61 |
| GhHT4-1-18 | 3674.84 | 13.2 |
| GhHT4-1-22 | 3703.25 | 14.08 |
| GhHT4-1-23 | 3672.64 | 13.14 |
| GhHT4-2-9 | 3521.48 | 8.48 |
| GhHT4-2-11 | 3503.13 | 7.91 |
| GhHT4-2-15 | 3609.13 | 11.18 |

Table 16 shows the results of the efficacy trials. All events performed well in the trials, with less than 10% injury after the herbicide treatments.

TABLE 16
Summary of the Fifth-Year Efficacy Trials Results

| | | Mean % Crop Injury | |
|---|---|---|---|
| | | 7 Days after | 14 Days after |
| Treatment | Event Name | Treatment | Treatment |
| Untreated Control | Wild type | 1.56 | 0.91 |
| | Commercial check | 1.79 | 0.77 |
| | GhHT4-1-10 | 1.35 | 0.92 |
| | GhHT4-1-11 | 1.59 | 0.97 |
| | GhHT4-1-13 | 1.52 | 0.77 |
| | GhHT4-1-17 | 1.85 | 0.91 |
| | GhHT4-1-18 | 1.62 | 0.97 |
| | GhHT4-1-22 | 1.64 | 0.89 |
| | GhHT4-1-23 | 1.40 | 1.03 |
| | GhHT4-2-9 | 1.30 | 0.72 |
| | GhHT4-2-11 | 1.54 | 0.61 |
| | GhHT4-2-15 | 1.58 | 0.74 |
| 2X Glyphosate | Commercial check | 6.27 | 5.93 |
| | GhHT4-1-10 | 3.15 | 0.74 |
| | GhHT4-1-11 | 3.19 | 0.77 |
| | GhHT4-1-13 | 3.02 | 0.85 |
| | GhHT4-1-17 | 3.21 | 0.94 |
| | GhHT4-1-18 | 3.50 | 0.47 |
| | GhHT4-1-22 | 3.52 | 0.92 |
| | GhHT4-1-23 | 3.08 | 0.94 |
| | GhHT4-2-9 | 3.21 | 0.69 |
| | GhHT4-2-11 | 3.58 | 0.67 |
| | GhHT4-2-15 | 3.15 | 0.72 |
| 2X Glufosinate | Commercial check | 4.38 | 1.71 |
| | GhHT4-1-10 | 3.15 | 1.09 |
| | GhHT4-1-11 | 3.50 | 1.27 |
| | GhHT4-1-13 | 2.85 | 1.36 |
| | GhHT4-1-17 | 2.90 | 1.19 |
| | GhHT4-1-18 | 3.23 | 1.28 |
| | GhHT4-1-22 | 3.26 | 1.53 |
| | GhHT4-1-23 | 3.70 | 1.14 |
| | GhHT4-2-9 | 2.79 | 1.33 |
| | GhHT4-2-11 | 2.81 | 1.42 |
| | GhHT4-2-15 | 3.50 | 1.34 |
| 2X Dicamba | Commercial check | 3.77 | 2.07 |
| | GhHT4-1-10 | 2.54 | 1.15 |
| | GhHT4-1-11 | 3.08 | 1.21 |
| | GhHT4-1-13 | 2.83 | 0.97 |
| | GhHT4-1-17 | 2.56 | 1.39 |
| | GhHT4-1-18 | 2.98 | 1.34 |
| | GhHT4-1-22 | 2.98 | 1.44 |
| | GhHT4-1-23 | 3.02 | 1.67 |
| | GhHT4-2-9 | 2.83 | 1.05 |
| | GhHT4-2-11 | 3.08 | 1.48 |
| | GhHT4-2-15 | 3.04 | 1.08 |
| 2X Mesotrione | GhHT4-1-10 | 2.61 | 1.21 |
| | GhHT4-1-11 | 2.76 | 1.23 |
| | GhHT4-1-13 | 2.84 | 1.23 |
| | GhHT4-1-17 | 2.92 | 1.28 |
| | GhHT4-1-18 | 2.82 | 1.26 |
| | GhHT4-1-22 | 2.88 | 0.95 |
| | GhHT4-1-23 | 2.53 | 1.15 |
| | GhHT4-2-9 | 2.31 | 1.62 |
| | GhHT4-2-11 | 2.73 | 1.08 |
| | GhHT4-2-15 | 2.76 | 0.97 |
| 2X Epyrifenacil | GhHT4-1-10 | 2.27 | 1.36 |
| | GhHT4-1-11 | 2.25 | 1.18 |
| | GhHT4-1-13 | 2.38 | 1.10 |
| | GhHT4-1-17 | 2.12 | 1.41 |
| | GhHT4-1-18 | 2.29 | 1.33 |
| | GhHT4-1-22 | 1.90 | 1.21 |
| | GhHT4-1-23 | 2.23 | 1.28 |
| | GhHT4-2-9 | 2.28 | 1.26 |
| | GhHT4-2-11 | 1.90 | 1.36 |
| | GhHT4-2-15 | 2.25 | 1.18 |

### Multi-Year Meta Analysis of Yield

To compare the field trial data and obtain more precise estimate on the performance of the lead transgenic events, a statistical meta-analysis was performed using the aggregate of all plants for multiple lead events in the 3^{rd}, 4^{th} and 5^{th} year, multi-location field trial data. Table 17 and Table 18 provide the meta-analysis results for yield in the agronomic and efficacy trials. Yield is presented in pound per acre of total seed cotton for the events, or for the untransformed control (wildtype) and a commercial check (Bollgard II® XtendFlex®). Percent crop injury was measured 7 and 14 days after treatment (DAT). SD_Mean refers to standard deviation of the mean. Based on the results of efficacy and agronomics trials, meta-analysis, molecular assessment, and breeding considerations, GhHT4-1-18 was selected as the commercial event and was named Gh_CSM63718.

TABLE 17
Meta-Analysis of Yield from Agronomic Field Trials

| Event Name | Yield (lb/acre Total Seed Cotton) | % Delta |
|---|---|---|
| WT | 3431.87 | — |
| Commercial check | 3813.79 | 11.13 |
| GhHT4-1-10 | 3626.23 | 5.66 |
| GhHT4-1-11 | 3665.90 | 6.82 |
| GhHT4-1-13 | 3661.53 | 6.69 |
| GhHT4-1-17 | 3570.00 | 4.02 |
| GhHT4-1-18 | 3609.24 | 5.17 |
| GhHT4-1-22 | 3676.68 | 7.13 |
| GhHT4-1-23 | 3619.91 | 5.48 |
| GhHT4-2-9 | 3434.30 | 0.07 |
| GhHT4-2-11 | 3424.75 | −0.21 |
| GhHT4-2-15 | 3515.14 | 2.43 |

TABLE 18
Meta-Analysis of Crop Injury from Efficacy Field Trials

| | | % Injury 7 DAT | | % Injury 14 DAT | |
|---|---|---|---|---|---|
| Treatment | Event Name | Mean | SD_Mean | Mean | SD_Mean |
| Glyphosate | Commercial check | 6.27 | 11.78 | 5.93 | 21.85 |
| | GhHT4-1-10 | 3.15 | 5.41 | 0.74 | 1.85 |
| | GhHT4-1-11 | 3.19 | 5.24 | 0.77 | 2.02 |
| | GhHT4-1-13 | 3.02 | 5.19 | 0.85 | 2.11 |
| | GhHT4-1-17 | 3.21 | 5.69 | 0.94 | 2.39 |
| | GhHT4-1-18 | 3.50 | 5.79 | 0.47 | 1.21 |
| | GhHT4-1-22 | 3.52 | 5.90 | 0.92 | 2.48 |
| | GhHT4-1-23 | 3.08 | 5.16 | 0.94 | 2.50 |
| | GhHT4-2-9 | 3.21 | 5.17 | 0.69 | 1.75 |
| | GhHT4-2-11 | 3.58 | 5.78 | 0.67 | 1.72 |
| | GhHT4-2-15 | 3.15 | 5.25 | 0.72 | 1.83 |
| Glufosinate | Commercial check | 4.38 | 7.90 | 1.71 | 3.96 |
| | GhHT4-1-10 | 3.15 | 4.55 | 1.09 | 2.45 |
| | GhHT4-1-11 | 3.50 | 5.57 | 1.27 | 2.79 |
| | GhHT4-1-13 | 2.85 | 4.39 | 1.36 | 3.16 |
| | GhHT4-1-17 | 2.90 | 4.50 | 1.19 | 2.65 |
| | GhHT4-1-18 | 3.23 | 4.90 | 1.28 | 2.80 |
| | GhHT4-1-22 | 3.26 | 5.40 | 1.53 | 3.23 |
| | GhHT4-1-23 | 3.70 | 5.11 | 1.14 | 2.47 |
| | GhHT4-2-9 | 2.79 | 4.58 | 1.33 | 2.81 |
| | GhHT4-2-11 | 2.81 | 4.64 | 1.42 | 3.36 |
| | GhHT4-2-15 | 3.50 | 5.47 | 1.34 | 3.09 |
| Dicamba | Commercial check | 3.77 | 5.97 | 2.07 | 3.88 |
| | GhHT4-1-10 | 2.54 | 4.19 | 1.15 | 2.40 |
| | GhHT4-1-11 | 3.08 | 4.93 | 1.21 | 2.53 |
| | GhHT4-1-13 | 2.83 | 4.51 | 0.97 | 2.02 |
| | GhHT4-1-17 | 2.56 | 4.20 | 1.39 | 2.83 |
| | GhHT4-1-18 | 2.98 | 5.27 | 1.34 | 2.75 |
| | GhHT4-1-22 | 2.98 | 5.02 | 1.44 | 2.92 |
| | GhHT4-1-23 | 3.02 | 4.34 | 1.67 | 3.17 |
| | GhHT4-2-9 | 2.83 | 4.53 | 1.05 | 2.25 |
| | GhHT4-2-11 | 3.08 | 5.05 | 1.48 | 3.01 |
| | GhHT4-2-15 | 3.04 | 4.58 | 1.08 | 2.16 |
| Mesotrione | GhHT4-1-10 | 2.61 | 4.67 | 1.21 | 2.56 |
| | GhHT4-1-11 | 2.76 | 5.02 | 1.23 | 2.56 |
| | GhHT4-1-13 | 2.84 | 4.96 | 1.23 | 2.45 |
| | GhHT4-1-17 | 2.92 | 4.89 | 1.28 | 2.63 |
| | GhHT4-1-18 | 2.82 | 4.76 | 1.26 | 2.58 |
| | GhHT4-1-22 | 2.88 | 4.95 | 0.95 | 1.95 |
| | GhHT4-1-23 | 2.53 | 4.29 | 1.15 | 2.35 |
| | GhHT4-2-9 | 2.31 | 4.16 | 1.62 | 3.85 |
| | GhHT4-2-11 | 2.73 | 4.76 | 1.08 | 2.34 |
| | GhHT4-2-15 | 2.76 | 4.89 | 0.97 | 1.93 |
| Epyrifenacil | GhHT4-1-10 | 2.27 | 3.91 | 1.36 | 2.63 |
| | GhHT4-1-11 | 2.25 | 3.97 | 1.18 | 2.40 |
| | GhHT4-1-13 | 2.38 | 4.38 | 1.10 | 2.22 |
| | GhHT4-1-17 | 2.12 | 3.83 | 1.41 | 2.91 |
| | GhHT4-1-18 | 2.29 | 4.28 | 1.33 | 2.67 |
| | GhHT4-1-22 | 1.90 | 3.59 | 1.21 | 2.35 |
| | GhHT4-1-23 | 2.23 | 4.02 | 1.28 | 2.57 |
| | GhHT4-2-11 | 1.90 | 3.75 | 1.36 | 2.72 |
| | GhHT4-2-15 | 2.25 | 4.06 | 1.18 | 2.32 |
| | GhHT4-3-1 | 2.28 | 4.34 | 1.26 | 3.35 |
| Untreated | Commercial check | 1.79 | 4.87 | 0.77 | 1.74 |
| | GhHT4-1-10 | 1.42 | 4.14 | 0.85 | 1.80 |
| | GhHT4-1-11 | 1.30 | 3.60 | 0.72 | 1.43 |
| | GhHT4-1-13 | 1.52 | 3.92 | 0.77 | 1.65 |
| | GhHT4-1-17 | 1.85 | 4.36 | 0.91 | 1.82 |
| | GhHT4-1-18 | 1.62 | 4.07 | 0.97 | 2.10 |
| | GhHT4-1-22 | 1.39 | 3.77 | 0.77 | 1.90 |
| | GhHT4-1-23 | 1.59 | 4.01 | 0.97 | 2.10 |
| | GhHT4-2-11 | 1.53 | 3.60 | 0.84 | 1.76 |
| | GhHT4-2-15 | 1.58 | 4.04 | 0.74 | 1.46 |
| | GhHT4-3-1 | 1.54 | 3.99 | 0.61 | 1.31 |

### Example 3: Cotton Event Gh_CSM63718 Provides Similar Agronomics and Fiber Quality Compared to the Wild-Type Control

As demonstrated in Example 2, Gh_CSM63718 provided similar yield of cotton compared to the wild-type control in multiple years in field trials, and through meta-analysis. In many cases, the event provided a yield advantage compared to the wild-type control. In addition to yield evaluation, several agronomic traits were also assessed. Examples of agronomic traits include early vigor score (EVS), plant height (PHT), percent open boll (POB), and lint percent (LP). Early vigor score is determined at approximately ten (10) to fourteen (14) days after the plants have emerged. The score is a visual rating used to determine if the plot has full yield potential based upon the emergence of the plants in the plot. The plots were rated using a scale of one to five. A rating of four or five indicates low vigor, while a rating of one corresponds to high vigor. Percent open boll is associated with maturity. It is a useful tool to determine when to defoliate cotton at the end of the season and is calculated as (# of opened first position/total # of first position bolls)×100. Lint percent of seed cotton refers to the ratio of lint mass to cotton seed mass and is calculated as ((total seed cotton mass—total cotton seed mass)/total seed cotton mass)×100. Total seed cotton includes cotton seed and lint before ginning to separate the lint from the seed, whereas total cotton seed refers to the seed without the lint. Table 19 shows the values of agronomic traits between event Gh_CSM63718 and the wild-type control for early vigor, plant height, percent open boll and lint percent. The early vigor score is based on two years of field trial data, and the rest are based on three years of field trial data. Each of the values are expressed as an average. “SE” stands for standard error. As shown in Table 19, transgenic cotton event Gh_CSM6318 demonstrated similar agronomic traits as the wild-type control.

TABLE 19
Agronomic Traits of Event Gh_CSM63718

| Event Name | EVS | SE | PHT | SE | POB | SE | LP | SE |
|---|---|---|---|---|---|---|---|---|
| Wild-type control | 1.64 | 0.53 | 35.76 | 2.04 | 64.62 | 3.82 | 37.93 | 0.36 |
| Gh_CSM63718 | 1.51 | 0.54 | 37.48 | 2.08 | 66.48 | 4.08 | 37.89 | 0.41 |

Twenty-five (25) bolls per plot were collected prior to harvest for assessment of fiber quality and other characteristics. Bolls were collected from a representative spot in the planting row. All bolls were harvested from the selected plants to prevent the introduction of any bias in the collection due to size or other characteristics. Fiber quality characteristics such as fiber strength, fiber length, length uniformity and micronaire were determined from the harvested bolls.

Fiber length was measured in inches and reported in 32nds of an inch.

Fiber strength was measured by breaking the fibers held between clamp jaws. It is reported as grams per tex, which is the force in grams required to break a bundle of fibers one tex unit in size. A tex unit is equal to the weight in grams of one thousand meters of fiber.

Length uniformity is the ratio between the mean length and the upper half mean length of the fibers, expressed as a percentage. Both the mean length and upper half mean length measurements are taken when the fibers were passed through a length sensor. Due to natural variation in the length of cotton fibers, length uniformity will always be less than 100 percent.

Micronaire is a measure of fiber fineness and maturity. It is the measurement of airflow resistance through a 2.34-gram fiber specimen that is compressed to a specific volume and is a unit-less value. Micronaire measurements can be influenced during the growing period by environmental conditions such as moisture, temperature, sunlight, plant nutrients, and extremes in plant or boll population. Favorable growing conditions result in fully mature fibers with premium range micronaire readings. Unfavorable conditions, such as lack of moisture, early freeze, or any other conditions that interrupt plant processes, will result in immature fibers and low micronaire measurements. High micronaire cotton is caused by such things as abnormally warm temperatures during boll maturation, or poor boll set leading to excessive availability of carbohydrates and over-maturing of fibers. Fiber fineness affects processing performance and the quality of the end-product in several ways. In the opening, cleaning, and carding processes, low micronaire or fine-fiber cottons require slower processing speeds to prevent damage to the fibers. Yams made from finer fiber result in more fibers per cross section, which in turn produces stronger yams. High micronaire or coarse fibers are not suitable for fine yams since the result would be fewer fibers per cross section, which would reduce the yam strength. Micronaire and maturity are highly correlated within a cotton variety. Dye absorbency and retention varies with the maturity of the fibers. Low maturity fibers have poor dye absorbency and retention while higher micronaire fibers have good absorbency and retention.

Table 20 provides a list of the fiber traits and their quality classifications. “V_MIN” stands for minimal value, whereas “V_MAX” stands for maximal value.

TABLE 20
Cotton Fiber Traits and Quality Classification
for America upland cotton

| Fiber Trait | V_MIN | V_MAX | Classification |
|---|---|---|---|
| Fiber Length | 1.088 | 1.148 | Poor |
| (Inches) | 1.150 | 1.169 | Average |
| | 1.170 | 1.188 | Good |
| | 1.191 | 1.219 | Very good |
| | 1.220 | 1.260 | Superior |
| Micronaire | 3.702 | 4.397 | Low |
| | 4.401 | 4.449 | Good |
| | 4.469 | 4.697 | Excellent |
| | 4.715 | 4.914 | Acceptable |
| Fiber Strength | 23.000 | 26.000 | Weak |
| (Grams/Tex) | 26.001 | 30.980 | Average |
| | 31.128 | 32.479 | Good |
| | 32.519 | 35.812 | Excellent |
| Length | 82.412 | 82.951 | Low |
| Uniformity | 83.023 | 83.393 | Good |
| (%) | 83.406 | 83.976 | Very good |
| | 84.004 | 84.340 | Superior |

Table 21 shows the fiber quality trait measurements for the wild-type control and event Gh_CSM63718 in the fifth-year field trials. No significant difference was observed in fiber length, strength, length uniformity and micronaire between the event and the wild-type control. Event Gh_CSM63718 and the wild-type control produced fibers of very similar qualities. These results demonstrate that fiber quality is not adversely affected by the expression of the transgene cassettes in event Gh_CSM63718.

TABLE 21
Fiber Quality of Event Gh_CSM63718

| Fiber Trait | Wild-Type Control | Gh_CSM63718 |
|---|---|---|
| Fiber Length | 1.17 | 1.17 |
| Fiber Strength | 29.69 | 28.86 |
| Length Uniformity | 82.82 | 86.66 |
| Micronaire | 4.32 | 4.16 |

### Example 4: Cre-Excision of the aadA and Cre Cassettes and ISR1 in Cotton Event Gh_CSM63718

This Example describes the removal of the aadA, Cre and ISR1 cassettes from cotton event Gh_CSM63718 through in vivo Cre-excision. By removal of the selection cassette, the Cre cassette and the ISR1, a “marker-free” event was created wherein only the herbicide tolerance gene expression cassettes remained in the final event.

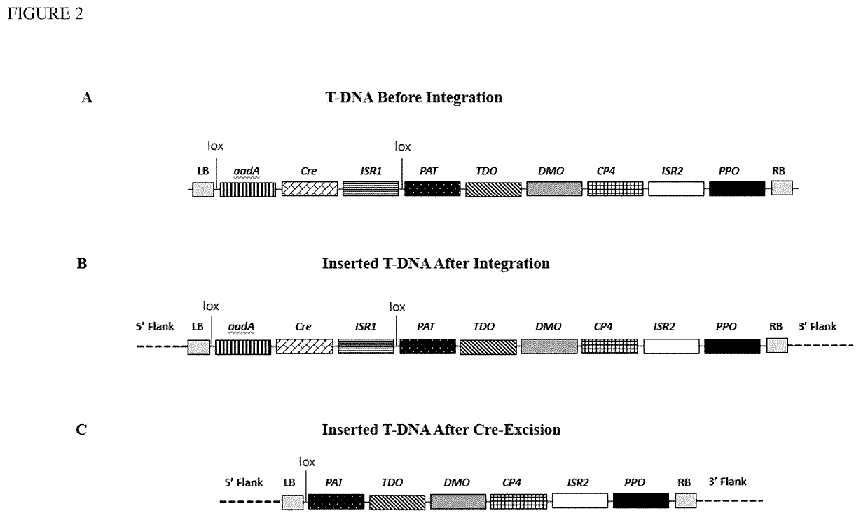

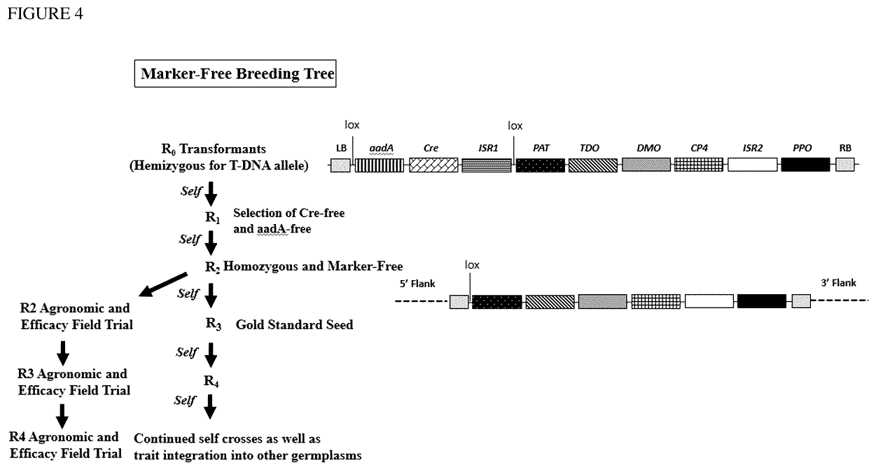

FIG. 4 illustrates the breeding process used to generate the marker-free Gh_CSM63718 cotton event. Construct GhHT4-1 comprises an aadA cassette, a Cre cassette and an intergenic sequence region (ISR1). The aadA cassette was used to select transformed cotton events resistant to spectinomycin/streptomycin during Agrobacterium-mediated transformation. The Cre cassette encodes a CRE recombinase from Enterobacteria phage P1 under the control of an anther specific promoter for removal of the aadA cassette, the Cre cassette and the ISR1. ISR1 is a non-coding, biologically neutral intergenic sequence designed to minimize potential effects of neighboring genes on each other's expression. The sequence of aadA, Cre and ISR1 in Gh_HT4-1 was flanked on both sides with Cre-recombinase LoxP recognition sites. As described in Example 1, following transformation of cotton seed-derived embryo explants with Agrobacterium-mediated transformation, R0 transgenic events were generated. The R0 events were self-pollinated to produce R1 seeds. The Cre-recombinase expressed by the Cre cassette under the control of an anther specific promoter binds to the LoxP sites and catalyzes a crossover in an eight-base pair spacer region of the two LoxP sites that flank the aadA, and Cre cassettes, and the ISR1, resulting in excision of the aadA and Cre cassttes, and the ISR1 with one LoxP site remaining in the integrated T-DNA due to recombination (see FIG. 2C, “Inserted T-DNA After Cre-Excision”).

The resulting R1 progenies were selected for the absence of the aadA and Cre cassettes, and the ISR1 (referred to as “marker free”) and allowed to self-pollinate to produce R2 progenies. The R2 progenies were selected which were marker free and homozygous for SEQ ID NO: 9, the transgenic T-DNA insert after Cre-excision. These selected R2 progenies were self-pollinated further for multiple generations, giving rise to the R3, R4, R5, and subsequent generations homozygous for SEQ ID NO: 9.

Homozygous R2 progenies and subsequent generations were used in field trials to assess their efficacy for herbicide tolerance and agronomic performance. R3 seeds and subsequent generations were assayed for purity and were designated as “Gold Standard Seed (GSS)”. Therefore, R3 was the first generation of gold standard seed. Gold Standard Seed is seed that has been assayed for purity to assure the absence of events other than cotton event Gh_CSM63718, and for molecular generation stability. The R4 or subsequent generations may be crossed to other cotton plants having a different genotype or germplasm, such as to allow for trait integration of the event, in other germplasms or genetic backgrounds and/or with other traits.

Excision of aadA, Cre and ISR1 cassettes did not affect the expression of the herbicide tolerance proteins encoded by SEQ ID NO:9. Removing the aadA and Cre cassettes, and the ISR1 from cotton event Gh_CSM63718 through Cre-excision provided a transgenic cotton event which conferred tolerance to multiple herbicides with different modes of action without adding the aadA cassette in the final event. This “marker-free” event assures flexibility when building cotton breeding stacks with other cotton transgenic events to provide a multiplicity of products incorporating cotton event Gh_CSM63718 and allowing multiple options for providing additional traits in the final commercial breeding stacks.

### Example 5: Molecular Characterization of Cotton Event Gh_CSM63718

As described in the preceding examples, cotton event Gh_CSM63718 was identified through comprehensive and rigorous event selection processes including molecular characterization and multi-year field trial assessment carried out from R0 to R5 generations. This example describes the extensive molecular characterization upon selection of Gh_CSM63718 as the commercial event, including confirmation of one copy of intact T-DNA at a single locus, absence of Agrobacterium Ti plasmid backbone DNA, aadA, Cre and intergenic sequence region 1 (ISR1) sequences; confirmation of the chromosomal location of the inserted T-DNA, confirmation that the T-DNA did not interrupt any known endogenous gene and did not insert into any repeat regions; and identification of the transgene 5′ and 3′ genomic flanking sequences and the wildtype allele sequence. The transgenic insert of cotton event Gh_CSM63718 contains the elements and sequences described in Table 1.

Molecular characterization of the cotton event Gh_CSM63718 was conducted initially in R0 plants using whole genome sequencing technology. The T-DNA genomic insertion site and molecular characteristics of the event were further confirmed in R1 and R2 generation plants and in R3 generation seeds. Molecular characterization from all four generations (R0, R1, R2, R3) demonstrated that event Gh_CSM63718 contained a single copy of the inserted T-DNA on chromosome 21 without any transformation vector backbone sequence, or the aadA, Cre or ISR1 sequences which was removed by auto excision at R1 seed.

Amplicons covering the transgenic insert and the 5′ or the 3′ flanking sequence were generated by PCR. The transgenic insertion site, and the respective 5′ and 3′ junction sequences, were subsequently determined by amplicon sequencing. The resulting event locus comprised from 5′ to 3′ a 1000-nucleotide left (5′) flanking genomic sequence, a stretch of 29 nucleotides inserted during T-DNA integration, a 16736-nucleotide T-DNA comprising five expression cassettes, a stretch of 31 nucleotides inserted during T-DNA integration, and a 1000-nucleotide right (3′) flanking genomic sequence (see FIG. 1 and Table 1). The wildtype (WT) allele sequence corresponding to the event Gh_CSM63718 transgenic insertion site was also determined from the wildtype genomic DNA by PCR and amplicon sequencing. Comparison of the WT allele sequence with that of the event Gh_CSM63718 at the insertion site revealed that the transgenic insertion resulted in a 103-nucleotide genomic sequence deletion in the event. Sequence information for the transgenic insert, the 5′ and 3′ flanking sequences, and the 5′ and 3′ junctions are provided herein as SEQ ID NOs:1-10. The corresponding WT allele sequence is provided herein as SEQ ID NO:13.

RNA analysis of plants comprising the cotton event Gh_CSM63718 was conducted. Northern hybridization was performed on total RNA isolated from R2 seeds and R4 immature seeds. Both results confirmed RNA transcripts corresponding in size to single PAT, TDO, DMO, EPSPS and PPO mRNA products in cotton event Gh_CSM63718.

Protein analyses by ELISA, Western blot hybridization and N-terminal sequencing were also conducted on plants comprising cotton event Gh_CSM63718. Results from ELISA assays using leaf tissues collected from R0 to R5 generations at various plant development stages showed that leaf expression levels of the PAT, TDO, DMO, EPSPS and PPO proteins were consistent and stable throughout the development stages and over multiple plant generations. Protein expression by ELISA was also measured using R2, R3 and R4 generation seeds. Consistent but lower levels of the PAT, TDO, DMO, EPSPS and PPO proteins were observed when compared to the levels from leaf tissues.

Western blot analysis was performed to evaluate size and level of PAT, TDO, DMO, EPSPS and PPO expressed in cotton event Gh_CSM63718 seed and leaf tissues. A single immunoreactive band at expected molecular weight was detected for all five genes of interest from event Gh_CSM63718 samples, indicating that cotton event Gh_CSM63718 produced the correct full-length size PAT, TDO, DMO, EPSPS and PPO proteins, consistent with the Northern blot results.

The N-terminal amino acid sequences of the expressed proteins were determined by Edman sequencing and mass spectrometry using immunopurified protein extracts from mature seed to confirm the authentic N-terminal amino acid sequence.

Furthermore, in planta protein sequencing was performed to ensure product integrity. The amino acid sequences of the PAT, TDO, DMO, EPSPS and PPO proteins were deduced from the in planta T-DNA sequencing results of cotton event Gh_CSM63718. The deduced protein sequences were then aligned with the same gene of interest sequences derived from the transformation construct. The deduced in planta protein sequences from cotton event Gh_CSM63718 showed perfect matches to the corresponding gene of interest protein sequences from the transformation construct.

### Example 6: Detection of Cotton Event Gh_CSM63718

This example describes methods useful in identifying or detecting the presence of cotton event Gh_CSM63718. Detection of the event in a sample can be achieved using DNA, RNA, or protein detection techniques. Illustrative detection methods and materials are provided below.

### A) Cotton Event Gh_CSM63718 Event-Specific Endpoint TaqmanT Assays

An event-specific endpoint Applied Biosystems™ TaqMan thermal amplification method (Thermo Fisher Scientific) was developed to identify cotton event Gh_CSM63718 in a sample. The DNA primers and probe used in the endpoint assay for this example are shown in Table 22, although it will be appreciated by those of skill in the art that that other primers and probes may also be used.

TABLE 22
Primers and Probe for Cotton
Gh_CSM63718 Event-Specific Assay

| SEQ ID NO. | Name | Type |
|---|---|---|
| 19 | Primer SQ51787 | Event-specific |
| 20 | Primer SQ51702 | Event-specific |
| 21 | 6FAM ™ probe PB50308 | Event-specific |
| 16 | Primer SQ22496 | Internal control |
| 17 | Primer SQ22497 | Internal control |
| 18 | VIC ™ Probe PB50562 | Internal control |

TaqMan thermal amplifications use dual-labeled Taqman MGB (Minor Groove Binder) probes incorporating 5′ fluorescent reporter dye 6-FAM™ or VIC™ and a 3′ nonfluorescent quencher. For TaqMan MGB (Minor Groove Binder) probes, the 5′ exonuclease activity of Taq DNA polymerase cleaves the probe from the 5′-end, between the fluorophore and quencher. When hybridized to the target DNA strand, quencher and fluorophore are separated enough to produce a fluorescent signal, thus releasing fluorescence. The pair of primers when used with these reaction methods and the probe produce a DNA amplicon that is diagnostic for cotton event Gh_CSM63718. The controls for this analysis should include a positive control containing cotton event Gh_CSM63718, a negative control from a non-transgenic plant, and a negative control that contains no template DNA. Additionally, a control for the PCR reaction should optimally include internal control primers and an internal control probe specific to a single copy gene in the cotton genome. These assays are optimized for use with the Applied Biosystems GeneAmp® PCR System 9700 (Thermo Fisher Scientific) run at maximum speed, but other equipment may be used.

Examples of PCR reaction components and cycling conditions useful for the event-specific qualitative endpoint TaqMan PCR assay for cotton event Gh_CSM63718 are presented in Table 23 and Table 24. The extracted DNA template was a leaf DNA sample to be analyzed, a negative control (non-transgenic cotton DNA), no template (water) control, or a positive control containing cotton event Gh_CSM63718 DNA.

TABLE 23
Gh_CSM63718 Event-Specific Endpoint TaqMan ™ PCR
Reaction Components

| | Stock | | |
|---|---|---|---|
| | Concentration | Volume | Final |
| Reagent | (□M) | (□l) | Concentration |
| Reaction Volume | | 5 | |
| Master Mix | | 2.28 | 1 X |
| Event Specific Primer | 100 | 0.05 | 0.9_M |
| SQ51702 | | | |
| Event Specific Primer | 100 | 0.05 | 0.9_M |
| SQ51787 | | | |
| Event Specific 6FAM ™ | 100 | 0.01 | 0.2_M |
| probe PB50308 | | | |
| Internal Control Primer | 100 | 0.05 | 0.9_M |
| SQ22496 | | | |
| Internal Control Primer | 100 | 0.05 | 0.9_M |
| SQ22497 | | | |
| Internal Control VIC ™ | 100 | 0.01 | 0.2_M |
| Probe PB50562 | | | |
| Extracted DNA template | | 2.5 | |

TABLE 24
Endpoint TaqMan ™ Thermocycler Conditions

| Step No. | Cycle No. | Settings |
|---|---|---|
| 1 | 1 | 95° C., 20 seconds |
| 2 | 35 | 95° C., 3 seconds |
| | | 60° C., 20 seconds |

### B) Detection of Cotton Event Gh_CSM63718 Using Antibody

Another example of detection of cotton event Gh_CSM63718 involves the use of antibodies specific for the PAT, DMO, TDO, CP4, and PPO proteins encoded by cotton event Gh_CSM63718. For example, a detection kit comprising the PPO, the TDO, or combinations of antibodies can be used. Such a kit may utilize a lateral flow strip comprising reagents activated when the tip of the strip is contacted with an aqueous solution. Illustrative proteins for use in antibody production are the PAT, DMO, TDO, CP4, and PPO proteins encoded by the sequences provided in SEQ ID NO:9.

A protein detection method can be used to determine whether a sample is from a plant, seed, cell, or plant part comprising cotton event Gh_CSM63718. An antibody specific for any of the proteins or any combination of the proteins encoded by cotton event Gh_CSM63718 is used to detect a protein or proteins encoded by cotton event Gh_CSM63718 in a sample. A detection kit comprising an antibody or combination of antibodies specific for the protein(s) encoded by cotton event Gh_CSM63718 may utilize a lateral flow strip containing reagent activated when the tip of the strip is contacted with an aqueous solution. Samples of cotton tissues may be ground up and protein extracted for analysis using water or an aqueous buffer (for example, phosphate buffered saline containing detergent and bovine serum albumin). Following centrifugation, the aqueous supernatant is analyzed using the ELISA method in a sandwich format on a lateral flow strip containing an absorbent pad. Detection is activated by dipping the tip of the strip into the aqueous solution containing the sample to be tested.

The aqueous solution is carried up the strip by capillary action and solubilizes gold labeled antibody or antibodies on the strip. The gold-labeled antibody is specific for the one or more proteins encoded by cotton event Gh_CSM63718 and will bind to an epitope on the protein(s) in the sample to form an antibody-antigen complex. The gold labeled antibody-antigen complex is then carried up the strip to a nitrocellulose membrane. The membrane comprises a test line of immobilized antibody that binds to a second, separate epitope on the protein(s) encoded by cotton event Gh_CSM63718, causing a visible line to appear across the test strip if the protein encoded by cotton event Gh_CSM63718 is present in the sample.

### C) Detection of Cotton Event Gh_CSM63718 by Southern Analysis

Another method to detect the presence of cotton event Gh_CSM63718 in a plant sample is Southern analysis as generally understood in the art. One of skill in art, based on the present disclosure and description of cotton event Gh_CSM63718, would understand how to design Southern hybridization probe(s) specific for the event and a second Southern hybridization probe specific for a plant which is null for the event (wildtype). With Southern analysis, a signal detected only from the first Southern hybridization probe will be indicative of a plant positive for cotton event Gh_CSM63718; a signal detected only from the second Southern hybridization probe will be indicative that the DNA was extracted from a plant that is null for the event (wild-type).

### Example 7: Zygosity Assays for Cotton Event Gh_CSM63718

This example describes methods useful in determining the zygosity of event Gh_CSM63718. The zygosity assay determines whether a plant comprising cotton event Gh_CSM63718 is heterozygous or homozygous for the event or the wildtype allele. Illustrative detection methods and materials are provided below.

A zygosity assay was developed to determine whether a plant comprising cotton event Gh_CSM63718 is heterozygous or homozygous for the event allele. An amplification reaction assay can be designed using the sequence information provided herein. For example, such a PCR assay would include design of at least three primers: primer-1, primer-2 and primer-3, where primer-1 is specific to cotton genomic DNA on the 3′ flanking DNA of cotton event Gh_CSM63718 (for example, SEQ ID NO:19); primer-2 is specific to cotton event Gh_CSM63718 transgenic insert (for example, SEQ ID NO:20); and primer-3 is specific to the wildtype allele (for example, SEQ ID NO:22). When used as a primer pair in an amplification reaction, primer-1 with primer-2 will produce a PCR amplicon specific for cotton event Gh_CSM63718. When used as a primer pair in an amplification reaction, primer-1 with primer-3 will produce a PCR amplicon specific for wildtype allele. In a PCR reaction performed on cotton event Gh_CSM63718, the respective PCR amplicons generated from primer-1+primer-2 and those generated from primer-1+primer-3 will differ in sequence and size of the amplicon. When the three primers are included in a PCR reaction with DNA extracted from a plant homozygous for cotton event Gh_CSM63718, only the primer-1+primer-2 amplicon (specific for the cotton event Gh_CSM63718) will be generated. When the three primers are included in a PCR reaction with DNA extracted from a plant heterozygous for cotton event Gh_CSM63718, both the primer-1+primer-2 amplicon (specific for the cotton event Gh_CSM63718 insert) and the primer-1+primer-3 amplicon (specific for the wildtype allele or absence of the cotton event Gh_CSM63718 insert) will be generated. When the three primers are mixed together in a PCR reaction with DNA extracted from a plant that is null for cotton event Gh_CSM63718 (i.e., wild-type), only the primer-1+primer-3 amplicon (specific for the wild-type allele) will be generated. The amplicons produced using the PCR reaction may be identified or distinguished using any method known in the art.

Another zygosity assay for cotton event Gh_CSM63718 is a Tagman™ thermal amplification method. Two fluorescently labeled probes are included in addition to the primers as described in the proceeding section. Probe-1, containing a fluorescent label (for example, the 6-FAM™_labeled SEQ ID NO:21), is specific for cotton event Gh_CSM63718; whereas Probe-2, containing a different fluorescent label (for example, the VIC™_labeled SEQ ID NO:23), is specific for a wildtype cotton plant that is null for cotton event Gh_CSM63718.

When the three primers (SEQ ID NO:19, SEQ ID NO:20, and SEQ ID NO:22) and two probes (SEQ ID NO:21 and SEQ ID NO:23) are mixed together in a PCR reaction with DNA extracted from a plant homozygous for cotton event Gh_CSM63718, a fluorescent signal from the 6FAM™_labeled probe PB50308 (SEQ ID NO:21) is released, which is indicative of and diagnostic for a plant homozygous for cotton event Gh_CSM63718. When the three primers and two probes are mixed together in a PCR reaction with DNA extracted from a plant heterozygous for cotton event Gh_CSM63718, two distinct fluorescent signals are generated, one from the 6FAM™_labeled probe PB50308 (SEQ ID NO:21), and one from the VIC™-labeled probe PB50683 (SEQ ID NO:23). When the three primers and the two probes are mixed together in a PCR reaction with DNA extracted from a wildtype plant which is null for cotton event Gh_CSM63718, a fluorescent signal from the VIC™_labeled probe PB50683 (SEQ ID NO:23) is generated.

Examples of PCR reaction components and cycling conditions useful for TaqMan™ PCR zygosity assay for cotton event Gh_CSM63718 are presented in Table 25 and Table 26. The extracted DNA template was a DNA sample to be analyzed, a negative control (non-transgenic cotton DNA), no template (water) control, or a positive control containing cotton event Gh_CSM63718 DNA.

TABLE 25
Gh_CSM63718 Zygosity TaqMan ™ PCR Reaction Components

| | Stock | | |
|---|---|---|---|
| | Concen- | | Final |
| | tration | Volume | Concen- |
| Reagent | (□M) | (□l) | tration |
| Reaction Volume | | 5 | |
| 2× Master Mix | | 2.345 | 1 X |
| Event Specific Primer SQ51702 | 100 | 0.045 | 0.9 □M |
| (SEQ ID NO: 20) | | | |
| Event Specific Primer SQ51787 | 100 | 0.045 | 0.9 □M |
| (SEQ ID NO: 19) | | | |
| Event Specific 6FAM ™ probe | 100 | 0.01 | 0.2 □M |
| PB50308 (SEQ ID NO: 21) | | | |
| Wildtype allele primer SQ52074 | 100 | 0.045 | 0.9 □M |
| (SEQ ID NO: 22) | | | |
| Wildtype allele VIC ™ probe | 100 | 0.01 | 0.2 □M |
| PB50683 (SEQ ID NO: 23) | | | |
| Extracted DNA template | | 2.5 | |

TABLE 26
Zygosity TaqMan ™ Thermocycler Conditions

| Step No. | Cycle No. | Settings |
|---|---|---|
| 1 | 1 | 95° C., 20 seconds |
| 2 | 35 | 95° C., 3 seconds |
| | | 60° C., 20 seconds |

Another method to detect the presence and zygosity of cotton event Gh_CSM63718 in a plant sample is Southern blot analysis. One of skill in art would understand how to design a first Southern hybridization probe(s) specific for cotton event Gh_CSM63718 and a second Southern hybridization probe specific for a cotton plant which is null for the cotton event Gh_CMS63718 (wildtype). With Southern blot analysis, a signal detected only from the first Southern hybridization probe is indicative a plant homozygous for cotton event Gh_CSM63718; signals detected from both the first and the second hybridization probes is indicative of a plant heterozygous for cotton event Gh_CSM63718; and a signal detected only from the second Southern hybridization probe indicates that the DNA was extracted from a plant that is null for cotton event Gh_CSM63718 (wildtype).

### Example 8: Modification of Cotton Event Gh_CSM63718 with Genome Editing Techniques Using a Single Guide RNA

This example describes how one may alter or excise all or a part of the transgenic insertion present in cotton event Gh_CSM63718, as well as flanking genomic DNA segments, such as by making one or more insertions, deletions, substitutions, or transpositions using genomic editing techniques. For example, such alterations can be made using Clustered Regularly Interspersed Short Palindromic Repeats (CRISPR) editing systems comprising a single guide RNA by genome editing methods. Sequences useful in excision of the event Gh_CSM63718 transgenic insertion, or one or more of the expression cassettes within SEQ ID NO:9 or SEQ ID NO:10 can be introduced through genome editing using a variety of methods. In one embodiment, Clustered Regularly Interspersed Short Palindromic Repeats (CRISPR) editing systems comprising a CRISPR associated protein and cognate guide RNAs may be used for targeted excision. The CRISPR-associated protein is an RNA-guided nuclease and can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and one or more guide RNAs (gRNAs) can be introduced into a plant cell corresponding to cotton event Gh_CSM63718 to target a specific sequence within the transgene insertion locus. In one embodiment, the CRISPR nuclease system cleaves at two identical guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination of any thereof, at the genomic target site. One or more guide RNA hybridization sequences can be inserted within the event Gh_CSM63718 transgene insertion locus which can subsequently allow for the excision all or a portion of the transgene insertion from event Gh_CSM63718 or one or more of the expression cassettes within SEQ ID NO:9 or SEQ ID NO:10.

Sequences corresponding to the 5′ and 3′ flanking genomic sequences and the transgenic insert of event Gh_CSM63718 (SEQ ID NOs:11, 12, and 9, respectively) and the 5′ and 3′ junction regions (SEQ ID NOs:1-6) were scanned for potential originator guide RNA recognition sites (OgRRS). As used herein, the term “originator guide RNA recognition site” or “OgRRS” refers to an endogenous DNA polynucleotide in the flanking genomic sequence or the integrated transgenic polynucleotide comprising a protospacer adjacent motif (PAM) site operably linked to a guide RNA hybridization site (i.e., protospacer sequence). In some embodiments, an OgRRS can be located in the flanking 5′ or 3′ genomic sequence (i.e., in non-transgenic DNA of a junction polynucleotide). In some embodiments, an OgRRS can be located in the 5′ or 3′ junction region (i.e., in both transgenic DNA and non-transgenic DNA of a junction polynucleotide or spanning transgenic and non-transgenic DNA in a DNA junction polynucleotide). In some embodiments, an OgRRS can be located in the transgenic insert. The OgRRS can be determined based upon the specific CRISPR editing system chosen. For example, Cas9 recognizes a G-rich protospacer-adjacent motif (PAM) that is 3′ to its guide RNA hybridization site whereas Cas12a systems recognize a T-rich protospacer-adjacent motif (PAM) that is 5′ to its guide RNA hybridization site.

The OgRRS sequence is then used to define a cognate guide RNA recognition site (CgRRS) which is inserted into the transgenic insertion locus of event Gh_CSM63718 using a CRISPR editing system. As used herein, the term “cognate guide RNA recognition site” or “CgRRS” refers to a DNA polynucleotide comprising a PAM site operably linked to a guide RNA hybridization site (i.e., protospacer sequence), where the CgRRS is absent from event Gh_CSM63718 comprising the original transgenic locus that is unmodified and where the CgRRS and its corresponding OgRRS can hybridize to a single gRNA, A CgRRS can be located in the flanking 5′ or 3′ genomic sequence (i.e., in non-transgenic DNA of a junction polynucleotide), in the 5′ or 3′ junction region (i.e., in both transgenic DNA and non-transgenic DNA of a junction polynucleotide or spanning transgenic and non-transgenic DNA in a DNA junction polynucleotide), or in the transgenic insert. A CgRRS comprises the same gRNA target sequence as the corresponding OgRRS. The CgRRS is inserted in a region within the transgenic insertion locus of event Gh_CSM63718 that is on the opposite side of the transgenic insertion, relative to the OgRRS in a manner that will permit the excision of a fragment of DNA corresponding to either the entire transgenic insertion of event Gh_CSM63718, or a fragment within the transgene insert of event Gh_CSM63718 such as an expression cassette or genetic element within the transgene cassette, using a single gRNA. For example, if the OgRRS is located within the 3′ flanking genomic sequence or the 3′ junction region, then the CgRRS can be inserted within the 5′ flanking genomic sequence, or the 5′ junction region, or within the transgene insert such as between genetic elements within an expression cassette, or within the transgenic insert such as between expression cassettes. Insertion of the CgRRS on the opposite side of the transgenic insertion or within the transgenic insert, relative to the OgRRS allows for excision of the transgenic insertion or specific expression element(s) or expression cassette(s) to be excised using a single gRNA. An OgRRS located in the expression cassette of event Gh_CSM63718 can be used to design a CgRRS that can be inserted in either the 5′ or 3′-flanking genomic sequence or in or near another expression cassette to permit excision of one or more expression elements or the expression cassette(s) using a single gRNA.

Table 27 shows OgRRS sequences located within the 5′ and 3′-flanking genomic sequences, spanning the 5′ and 3′ junction sequences, and in the transgenic insertion of event Gh_CSM63718 that can be used in a CRISPR editing system employing Cas12a, a Type V CRISPR-associated protein. The analysis was performed for four Cas12a endonucleases. FnCas12a (SEQ ID NO:202) refers to Francisella novicida U112 Cas12a (also known as FnCpf1) and requires the PAM sequence of 5′-TTN, where N is A, C, G or T (Zetsche et al., 2015). LbCas12a (SEQ ID NO:199) refers to the Cas12a from Lachnospiraceae bacterium ND2006 (also known as LbCpf1) and requires the PAM sequence of 5′-TTTV, where V is A, C, or G. LbCas12a-TYC and LbCas12a-TAT refer to engineered variants of Lachnospiraceae bacterium ND2006 Cas12a (Gao et al., 2017). The LbCas12a-TYC variant (SEQ ID NO:200) contains the G532R/K595R mutations and recognizes 5′-TYCV PAM; whereas the LbCas12a-TAT variant (SEQ ID NO:201) contains the G532R/K538V/Y542R mutations and recognizes 5′-TATV PAM, where Y is C or T, and V is A, C or G. The PAM sequence, the coordinates of the gRNA hybridization site (also known as OgRRS) relative to SEQ ID NO:10, and the corresponding Cas12a endonuclease are shown under the headings of “PAM”, “Cas12a Nuclease”, and “Start . . . End of gRNA Hybridization Site in SED ID NO:10”, respectively. “Strand of SEQ ID NO:10” indicates whether the identified gRNA hybridization site along with its PAM sequence is on the forward strand (+) or the complementary strand (−). “Target Site” refers to the location of the gRNA hybridization site in the cotton event Gh_CSM63718 locus.

TABLE 27
OgRRS Sequences within Event Gh_CSM63718

| | | | Start . . . | | |
|---|---|---|---|---|---|
| Name of gRNA | | | End of gRNA | | |
| Hybridization | | | Hybridization | Strand | |
| Site | | Cas12a | Site in SEQ | of SEQ | Target |
| (OgRRS) | PAM | Nuclease | ID NO: 10 | ID NO: 10 | Site |
| 5F-1 | TTTC | LbCas12a | 20:42 | + | 5′ Flanking |
| | | | | | DNA |
| 5F-2 | TTCA | LbCas12a-TYC | 21:43 | + | 5′ Flanking |
| | | | | | DNA |
| 5F-3 | TTCA | LbCas12a-TYC | 25:47 | + | 5′ Flanking |
| | | | | | DNA |
| 5F-4 | CTTA | FnCas12a | 33:55 | + | 5′ Flanking |
| | | | | | DNA |
| 5F-5 | TATG | LbCas12a-TATV | 35:57 | + | 5′ Flanking |
| | | | | | DNA |
| 5F-6 | GTTC | FnCas12a | 39:61 | + | 5′ Flanking |
| | | | | | DNA |
| 5F-7 | TTCA | LbCas12a-TYC | 40:62 | + | 5′ Flanking |
| | | | | | DNA |
| 5F-8 | GTTA | FnCas12a | 47:69 | + | 5′ Flanking |
| | | | | | DNA |
| 5F-9 | CTTA | FnCas12a | 61:83 | + | 5′ Flanking |
| | | | | | DNA |
| 5F-10 | TATG | LbCas12a-TATV | 63:85 | + | 5′ Flanking |
| | | | | | DNA |
| 5F-11 | CTTG | FnCas12a | 51:73 | − | 5′ Flanking |
| | | | | | DNA |
| 5F-12 | GTTG | FnCas12a | 59:81 | − | 5′ Flanking |
| | | | | | DNA |
| 5F-13 | CTTG | FnCas12a | 82:104 | + | 5′ Flanking |
| | | | | | DNA |
| 5F-14 | GTTA | FnCas12a | 245:267 | + | 5′ Flanking |
| | | | | | DNA |
| 5F-15 | GTTA | FnCas12a | 254:276 | − | 5′ Flanking |
| | | | | | DNA |
| 5F-16 | TTTC | LbCas12a | 267:289 | − | 5′ Flanking |
| | | | | | DNA |
| 5F-17 | TATA | LbCas12a-TATV | 305:327 | + | 5′ Flanking |
| | | | | | DNA |
| 5F-18 | CTTG | FnCas12a | 297:319 | − | 5′ Flanking |
| | | | | | DNA |
| 5F-19 | TTCA | LbCas12a-TYC | 322:344 | + | 5′ Flanking |
| | | | | | DNA |
| 5F-20 | TTTG | LbCas12a-TYC | 331:353 | + | 5′ Flanking |
| | | | | | DNA |
| 5F-21 | TTTA | LbCas12a | 337:359 | + | 5′ Flanking |
| | | | | | DNA |
| 5F-22 | TATA | LbCas12a-TATV | 320:342 | − | 5′ Flanking |
| | | | | | DNA |
| 5F-23 | TATA | LbCas12a-TATV | 347:369 | + | 5′ Flanking |
| | | | | | DNA |
| 5F-24 | TTCA | LbCas12a-TYC | 327:349 | − | 5′ Flanking |
| | | | | | DNA |
| 5F-25 | TTTC | LbCas12a | 328:350 | − | 5′ Flanking |
| | | | | | DNA |
| 5F-26 | TTTG | LbCas12a | 352:374 | + | 5′ Flanking |
| | | | | | DNA |
| 5F-27 | TCCA | LbCas12a-TYC | 365:387 | + | 5′ Flanking |
| | | | | | DNA |
| 5F-28 | TTTA | LbCas12a | 371:393 | + | 5′ Flanking |
| | | | | | DNA |
| 5F-29 | TTTG | LbCas12a | 356:378 | − | 5′ Flanking |
| | | | | | DNA |
| 5F-30 | TTCA | LbCas12a-TYC | 381:403 | + | 5′ Flanking |
| | | | | | DNA |
| 5F-31 | TATG | LbCas12a-TATV | 371:393 | − | 5′ Flanking |
| | | | | | DNA |
| 5F-32 | TTCA | LbCas12a-TYC | 396:418 | + | 5′ Flanking |
| | | | | | DNA |
| 5F-33 | TTTC | LbCas12a | 402:424 | + | 5′ Flanking |
| | | | | | DNA |
| 5F-34 | TTCC | LbCas12a-TYC | 403:425 | + | 5′ Flanking |
| | | | | | DNA |
| 5F-35 | TCCA | LbCas12a-TYC | 404:426 | + | 5′ Flanking |
| | | | | | DNA |
| 5F-36 | TTCC | LbCas12a-TYC | 408:430 | + | 5′ Flanking |
| | | | | | DNA |
| 5F-37 | TCCA | LbCas12a-TYC | 409:431 | + | 5′ Flanking |
| | | | | | DNA |
| 5F-38 | GTTG | FnCas12a | 388:410 | − | 5′ Flanking |
| | | | | | DNA |
| 5F-39 | CTTC | FnCas12a | 418:440 | + | 5′ Flanking |
| | | | | | DNA |
| 5F-40 | TCCA | LbCas12a-TYC | 401:423 | − | 5′ Flanking |
| | | | | | DNA |
| 5F-41 | TTCC | LbCas12a-TYC | 402:424 | − | 5′ Flanking |
| | | | | | DNA |
| 5F-42 | TTTC | LbCas12a | 403:425 | − | 5′ Flanking |
| | | | | | DNA |
| 5F-43 | TTTG | LbCas12a-TYC | 426:448 | + | 5′ Flanking |
| | | | | | DNA |
| 5F-44 | TATA | LbCas12a-TATV | 410:432 | − | 5′ Flanking |
| | | | | | DNA |
| 5F-45 | CTTA | FnCas12a | 412:434 | − | 5′ Flanking |
| | | | | | DNA |
| 5F-46 | TATC | LbCas12a-TATV | 415:437 | − | 5′ Flanking |
| | | | | | DNA |
| 5F-47 | TATA | LbCas12a-TATV | 437:459 | + | 5′ Flanking |
| | | | | | DNA |
| 5F-48 | TATG | LbCas12a-TATV | 430:452 | − | 5′ Flanking |
| | | | | | DNA |
| 5F-49 | GTTA | FnCas12a | 432:454 | − | 5′ Flanking |
| | | | | | DNA |
| 5F-50 | TTTA | LbCas12a | 436:458 | − | 5′ Flanking |
| | | | | | DNA |
| 5F-51 | TTTA | LbCas12a | 473:495 | + | 5′ Flanking |
| | | | | | DNA |
| 5F-52 | TTTA | LbCas12a | 479:501 | + | 5′ Flanking |
| | | | | | DNA |
| 5F-53 | TATG | LbCas12a-TATV | 481:503 | + | 5′ Flanking |
| | | | | | DNA |
| 5F-54 | GTTG | FnCas12a | 484:506 | + | 5′ Flanking |
| | | | | | DNA |
| 5F-55 | CTTC | FnCas12a | 489:511 | + | 5′ Flanking |
| | | | | | DNA |
| 5F-56 | TTCA | LbCas12a-TYC | 467:489 | − | 5′ Flanking |
| | | | | | DNA |
| 5F-57 | CTTG | FnCas12a | 492:514 | + | 5′ Flanking |
| | | | | | DNA |
| 5F-58 | TATC | LbCas12a-TATV | 504:526 | + | 5′ Flanking |
| | | | | | DNA |
| 5F-59 | TTTC | LbCas12a | 510:532 | + | 5′ Flanking |
| | | | | | DNA |
| 5F-60 | TTCC | LbCas12a-TYC | 511:533 | + | 5′ Flanking |
| | | | | | DNA |
| 5F-61 | TCCA | LbCas12a-TYC | 512:534 | + | 5′ Flanking |
| | | | | | DNA |
| 5F-62 | CTTG | FnCas12a | 522:544 | + | 5′ Flanking |
| | | | | | DNA |
| 5F-63 | TTTA | LbCas12a | 528:550 | + | 5′ Flanking |
| | | | | | DNA |
| 5F-64 | TTTG | LbCas12a | 538:560 | + | 5′ Flanking |
| | | | | | DNA |
| 5F-65 | TCCA | LbCas12a-TYC | 542:564 | − | 5′ Flanking |
| | | | | | DNA |
| 5F-66 | TTCC | LbCas12a-TYC | 543:565 | − | 5′ Flanking |
| | | | | | DNA |
| 5F-67 | CTTC | FnCas12a | 544:566 | − | 5′ Flanking |
| | | | | | DNA |
| 5F-68 | TTTG | LbCas12a | 553:575 | − | 5′ Flanking |
| | | | | | DNA |
| 5F-69 | TTCG | LbCas12a-TYC | 797:819 | + | 5′ Flanking |
| | | | | | DNA |
| 5F-70 | TTTA | LbCas12a | 803:825 | + | 5′ Flanking |
| | | | | | DNA |
| 5F-71 | TATC | LbCas12a-TATV | 805:827 | + | 5′ Flanking |
| | | | | | DNA |
| 5F-72 | TTTA | LbCas12a | 785:807 | − | 5′ Flanking |
| | | | | | DNA |
| 5F-73 | TCCC | LbCas12a-TYC | 807:829 | + | 5′ Flanking |
| | | | | | DNA |
| 5F-74 | TATG | LbCas12a-TATV | 790:812 | − | 5′ Flanking |
| | | | | | DNA |
| 5F-75 | TATA | LbCas12a-TATV | 792:814 | − | 5′ Flanking |
| | | | | | DNA |
| 5F-76 | GTTA | FnCas12a | 794:816 | − | 5′ Flanking |
| | | | | | DNA |
| 5F-77 | TATA | LbCas12a-TATV | 804:826 | − | 5′ Flanking |
| | | | | | DNA |
| 5F-78 | TTTA | LbCas12a | 806:828 | − | 5′ Flanking |
| | | | | | DNA |
| 5F-79 | TATA | LbCas12a-TATV | 831:853 | + | 5′ Flanking |
| | | | | | DNA |
| 5F-80 | TATA | LbCas12a-TATV | 839:861 | + | 5′ Flanking |
| | | | | | DNA |
| 5F-81 | GTTA | FnCas12a | 854:876 | + | 5′ Flanking |
| | | | | | DNA |
| 5F-82 | TTCA | LbCas12a-TYC | 836:858 | − | 5′ Flanking |
| | | | | | DNA |
| 5F-83 | TTTG | LbCas12a | 843:865 | − | 5′ Flanking |
| | | | | | DNA |
| 5F-84 | TTTA | LbCas12a-TATV | 851:873 | − | 5′ Flanking |
| | | | | | DNA |
| 5F-85 | TATA | LbCas12a-TATV | 887:909 | + | 5′ Flanking |
| | | | | | DNA |
| 5F-86 | TTTA | LbCas12a | 894:916 | + | 5′ Flanking |
| | | | | | DNA |
| 5F-87 | GTTA | FnCas12a | 903:925 | + | 5′ Flanking |
| | | | | | DNA |
| 5F-88 | TATA | LbCas12a-TATV | 889:911 | − | 5′ Flanking |
| | | | | | DNA |
| 5F-89 | TTTC | LbCas12a | 898:920 | − | 5′ Flanking |
| | | | | | DNA |
| 5F-90 | TTTA | LbCas12a | 929:951 | + | 5′ Flanking |
| | | | | | DNA |
| 5F-91 | TTCA | LbCas12a-TYC | 945:967 | − | 5′ Flanking |
| | | | | | DNA |
| 5F-92 | TTTC | LbCas12a | 946:968 | − | 5′ Flanking |
| | | | | | DNA |
| 5F-93 | TTTG | LbCas12a-TYC | 970:992 | + | 5′ Flanking |
| | | | | | DNA |
| 5F-94 | TTCG | LbCas12a-TYC | 958:980 | − | 5′ Flanking |
| | | | | | DNA |
| 5F-95 | TTTC | LbCas12a | 959:981 | − | 5′ Flanking |
| | | | | | DNA |
| 5F-96 | TTTA | LbCas12a | 966:988 | − | 5′ Flanking |
| | | | | | DNA |
| 5J-1 | TATA | LbCas12a-TATV | 999:1021 | + | 5′ Junction |
| 5J-2 | TTTC | LbCas12a | 994:1016 | − | 5′ Junction |
| TI-1 | TCCG | LbCas12a-TYC | 1071:1093 | + | Transgenic |
| | | | | | insert |
| TI-2 | TATA | LbCas12a-TATV | 1057:1079 | − | Transgenic |
| | | | | | insert |
| TI-3 | TTTA | LbCas12a | 1059:1081 | − | Transgenic |
| | | | | | insert |
| TI-4 | TTCA | LbCas12a-TYC | 1064:1086 | − | Transgenic |
| | | | | | insert |
| TI-5 | TTTC | LbCas12a | 1065:1087 | − | Transgenic |
| | | | | | insert |
| TI-6 | TTTG | LbCas12a | 1102:1124 | + | Transgenic |
| | | | | | insert |
| TI-7 | TTTA | LbCas12a | 1080:1102 | − | Transgenic |
| | | | | | insert |
| TI-8 | TTTG | LbCas12a | 1088:1110 | − | Transgenic |
| | | | | | insert |
| TI-9 | TCCG | LbCas12a-TYC | 1125:1147 | + | Transgenic |
| | | | | | insert |
| TI-10 | TATA | LbCas12a-TATV | 1109:1131 | − | Transgenic |
| | | | | | insert |
| TI-11 | TTTA | LbCas12a | 1134:1156 | + | Transgenic |
| | | | | | insert |
| TI-12 | TATA | LbCas12a-TATV | 1136:1158 | + | Transgenic |
| | | | | | insert |
| TI-13 | TTCG | LbCas12a-TYC | 1115:1137 | − | Transgenic |
| | | | | | insert |
| TI-14 | TTTC | LbCas12a | 1116:1138 | − | Transgenic |
| | | | | | insert |
| TI-15 | TTTA | LbCas12a | 1124:1146 | − | Transgenic |
| | | | | | insert |
| TI-16 | TTTG | LbCas12a | 1128:1150 | − | Transgenic |
| | | | | | insert |
| TI-17 | TCCG | LbCas12a-TYC | 1143:1165 | − | Transgenic |
| | | | | | insert |
| TI-18 | TTCC | LbCas12a-TYC | 1144:1166 | − | Transgenic |
| | | | | | insert |
| TI-19 | TTTC | LbCas12a | 1145:1167 | − | Transgenic |
| | | | | | insert |
| TI-20 | TTCG | LbCas12a-TYC | 1168:1190 | + | Transgenic |
| | | | | | insert |
| TI-21 | TTTA | LbCas12a | 1178:1200 | + | Transgenic |
| | | | | | insert |
| TI-22 | TTTC | LbCas12a | 1182:1204 | + | Transgenic |
| | | | | | insert |
| TI-23 | TTCG | LbCas12a-TYC | 1183:1205 | + | Transgenic |
| | | | | | insert |
| TI-24 | TTTG | LbCas12a | 1179:1201 | − | Transgenic |
| | | | | | insert |
| TI-25 | CTTA | FnCas12a | 1216:1238 | + | Transgenic |
| | | | | | insert |
| TI-26 | TATA | LbCas12a-TATV | 1208:1230 | − | Transgenic |
| | | | | | insert |
| TI-27 | GTTA | FnCas12a | 1210:1232 | − | Transgenic |
| | | | | | insert |
| TI-28 | TATA | LbCas12a-TATV | 1233:1255 | + | Transgenic |
| | | | | | insert |
| TI-29 | TATA | LbCas12a-TATV | 1235:1257 | + | Transgenic |
| | | | | | insert |
| TI-30 | CTTC | FnCas12a | 1240:1262 | + | Transgenic |
| | | | | | insert |
| TI-31 | TATA | LbCas12a-TATV | 1218:1240 | − | Transgenic |
| | | | | | insert |
| TI-32 | TTCG | LbCas12a-TYC | 1241:1263 | + | Transgenic |
| | | | | | insert |
| TI-33 | TATA | LbCas12a-TATV | 1245:1267 | + | Transgenic |
| | | | | | insert |
| TI-34 | TATA | LbCas12a-TATV | 1230:1252 | − | Transgenic |
| | | | | | insert |
| TI-35 | TATG | LbCas12a-TATV | 1252:1274 | + | Transgenic |
| | | | | | insert |
| TI-36 | TTCG | LbCas12a-TYC | 1234:1256 | − | Transgenic |
| | | | | | insert |
| TI-37 | CTTC | FnCas12a | 1235:1257 | − | Transgenic |
| | | | | | insert |
| TI-38 | TATA | LbCas12a-TATV | 1257:1279 | + | Transgenic |
| | | | | | insert |
| TI-39 | GTTA | FnCas12a | 1265:1287 | + | Transgenic |
| | | | | | insert |
| TI-40 | TTTC | LbCas12a | 1269:1291 | + | Transgenic |
| | | | | | insert |
| TI-41 | TTCG | LbCas12a-TYC | 1270:1292 | + | Transgenic |
| | | | | | insert |
| TI-42 | CTTA | FnCas12a | 1287:1309 | + | Transgenic |
| | | | | | insert |
| TI-43 | CTTG | FnCas12a | 1277:1299 | − | Transgenic |
| | | | | | insert |
| TI-44 | TTTC | LbCas12a | 1312:1334 | + | Transgenic |
| | | | | | insert |
| TI-45 | TTCA | LbCas12a-TYC | 1313:1335 | + | Transgenic |
| | | | | | insert |
| TI-46 | TCCA | LbCas12a-TYC | 1294:1316 | − | Transgenic |
| | | | | | insert |
| TI-47 | TTTG | LbCas12a | 1319:1341 | + | Transgenic |
| | | | | | insert |
| TI-48 | GTTA | FnCas12a | 1311:1333 | − | Transgenic |
| | | | | | insert |
| TI-49 | GTTG | FnCas12a | 1316:1338 | − | Transgenic |
| | | | | | insert |
| TI-50 | TTCG | LbCas12a-TYC | 1319:1341 | − | Transgenic |
| | | | | | insert |
| TI-51 | GTTC | FnCas12a | 1320:1342 | − | Transgenic |
| | | | | | insert |
| TI-52 | GTTA | FnCas12a | 1326:1348 | − | Transgenic |
| | | | | | insert |
| TI-53 | TTTA | LbCas12a | 1337:1359 | − | Transgenic |
| | | | | | insert |
| TI-54 | TTTA | LbCas12a | 1342:1364 | − | Transgenic |
| | | | | | insert |
| TI-55 | TTTG | LbCas12a | 1385:1407 | + | Transgenic |
| | | | | | insert |
| TI-56 | TTTA | LbCas12a-TYC | 1363:1385 | − | Transgenic |
| | | | | | insert |
| TI-57 | TTTA | LbCas12a | 1375:1397 | − | Transgenic |
| | | | | | insert |
| TI-58 | TTTA | LbCas12a | 1389:1411 | − | Transgenic |
| | | | | | insert |
| TI-59 | GTTG | FnCas12a | 1432:1454 | − | Transgenic |
| | | | | | insert |
| TI-60 | TTTC | LbCas12a | 1456:1478 | + | Transgenic |
| | | | | | insert |
| TI-61 | CTTG | FnCas12a | 1435:1457 | − | Transgenic |
| | | | | | insert |
| TI-62 | TTCA | LbCas12a-TYC | 1457:1479 | + | Transgenic |
| | | | | | insert |
| TI-63 | TTTC | LbCas12a | 1438:1460 | − | Transgenic |
| | | | | | insert |
| TI-64 | TCCC | LbCas12a-TYC | 1444:1466 | − | Transgenic |
| | | | | | insert |
| TI-65 | TTCC | LbCas12a-TYC | 1445:1467 | − | Transgenic |
| | | | | | insert |
| TI-66 | TTTC | LbCas12a | 1446:1468 | − | Transgenic |
| | | | | | insert |
| TI-67 | TTTC | LbCas12a | 1450:1472 | − | Transgenic |
| | | | | | insert |
| TI-68 | TTTA | LbCas12a | 1460:1482 | − | Transgenic |
| | | | | | insert |
| TI-69 | TATG | LbCas12a-TATV | 1465:1487 | − | Transgenic |
| | | | | | insert |
| TI-70 | TTTA | LbCas12a | 1467:1489 | − | Transgenic |
| | | | | | insert |
| TI-71 | TATG | LbCas12a-TATV | 1494:1516 | − | Transgenic |
| | | | | | insert |
| TI-72 | TTTC | LbCas12a | 1529:1551 | + | Transgenic |
| | | | | | insert |
| TI-73 | TTCC | LbCas12a-TYC | 1530:1552 | + | Transgenic |
| | | | | | insert |
| TI-74 | TTTC | LbCas12a | 1534:1556 | + | Transgenic |
| | | | | | insert |
| TI-75 | CTTA | FnCas12a | 1537:1559 | + | Transgenic |
| | | | | | insert |
| TI-76 | TTTC | LbCas12a | 1545:1567 | + | Transgenic |
| | | | | | insert |
| TI-77 | TTCA | LbCas12a-TYC | 1546:1568 | + | Transgenic |
| | | | | | insert |
| TI-78 | TCCA | LbCas12a-TYC | 1525:1547 | − | Transgenic |
| | | | | | insert |
| TI-79 | TATC | LbCas12a-TATV | 1527:1549 | − | Transgenic |
| | | | | | insert |
| TI-80 | TTTA | LbCas12a | 1562:1584 | + | Transgenic |
| | | | | | insert |
| TI-81 | CTTG | FnCas12a | 1566:1588 | + | Transgenic |
| | | | | | insert |
| TI-82 | TCCA | LbCas12a-TYC | 1550:1572 | − | Transgenic |
| | | | | | insert |
| TI-83 | CTTG | FnCas12a | 1573:1595 | + | Transgenic |
| | | | | | insert |
| TI-84 | TTCC | LbCas12a-TYC | 1551:1573 | − | Transgenic |
| | | | | | insert |
| TI-85 | TTTA | LbCas12a | 1582:1604 | + | Transgenic |
| | | | | | insert |
| TI-86 | TATG | LbCas12a-TATV | 1584:1606 | + | Transgenic |
| | | | | | insert |
| TI-87 | GTTA | FnCas12a | 1587:1609 | + | Transgenic |
| | | | | | insert |
| TI-88 | TATG | LbCas12a-TATV | 1589:1611 | + | Transgenic |
| | | | | | insert |
| TI-89 | CTTG | FnCas12a | 1605:1627 | + | Transgenic |
| | | | | | insert |
| TI-90 | TATA | LbCas12a-TATV | 1584:1606 | − | Transgenic |
| | | | | | insert |
| TI-91 | TTTA | LbCas12a | 1586:1608 | − | Transgenic |
| | | | | | insert |
| TI-92 | TATA | LbCas12a-TATV | 1611:1633 | + | Transgenic |
| | | | | | insert |
| TI-93 | TTTA | LbCas12a | 1610:1632 | − | Transgenic |
| | | | | | insert |
| TI-94 | TTTA | LbCas12a-TYC | 1621:1643 | − | Transgenic |
| | | | | | insert |
| TI-95 | TTTA | LbCas12a | 1649:1671 | − | Transgenic |
| | | | | | insert |
| TI-96 | CTTA | FnCas12a | 1685:1707 | + | Transgenic |
| | | | | | insert |
| TI-97 | TTTA | LbCas12a | 1694:1716 | + | Transgenic |
| | | | | | insert |
| TI-98 | TTCC | LbCas12a-TYC | 1698:1720 | + | Transgenic |
| | | | | | insert |
| TI-99 | TTTG | LbCas12a | 1678:1700 | − | Transgenic |
| | | | | | insert |
| TI-100 | TATA | LbCas12a-TATV | 1710:1732 | + | Transgenic |
| | | | | | insert |
| TI-101 | TTCA | LbCas12a-TYC | 1715:1737 | + | Transgenic |
| | | | | | insert |
| TI-102 | TTTA | LbCas12a | 1724:1746 | + | Transgenic |
| | | | | | insert |
| TI-103 | TATG | LbCas12a-TATV | 1707:1729 | − | Transgenic |
| | | | | | insert |
| TI-104 | TTTA | LbCas12a | 1730:1752 | + | Transgenic |
| | | | | | insert |
| TI-105 | TTTA | LbCas12a-TYC | 1709:1731 | − | Transgenic |
| | | | | | insert |
| TI-106 | GTTC | FnCas12a | 1716:1738 | − | Transgenic |
| | | | | | insert |
| TI-107 | GTTG | FnCas12a | 1727:1749 | − | Transgenic |
| | | | | | insert |
| TI-108 | GTTG | FnCas12a | 1748:1770 | − | Transgenic |
| | | | | | insert |
| TI-109 | TTTA | LbCas12a | 1753:1775 | − | Transgenic |
| | | | | | insert |
| TI-110 | TATG | LbCas12a-TATV | 1805:1827 | + | Transgenic |
| | | | | | insert |
| TI-111 | TTTA | LbCas12a | 1811:1833 | + | Transgenic |
| | | | | | insert |
| TI-112 | TTTG | LbCas12a | 1794:1816 | − | Transgenic |
| | | | | | insert |
| TI-113 | GTTA | FnCas12a | 1804:1826 | − | Transgenic |
| | | | | | insert |
| TI-114 | GTTA | FnCas12a | 1813:1835 | − | Transgenic |
| | | | | | insert |
| TI-115 | TCCA | LbCas12a-TYC | 1823:1845 | − | Transgenic |
| | | | | | insert |
| TI-116 | CTTG | FnCas12a | 1848:1870 | + | Transgenic |
| | | | | | insert |
| TI-117 | TCCA | LbCas12a-TYC | 1832:1854 | − | Transgenic |
| | | | | | insert |
| TI-118 | TTCC | LbCas12a-TYC | 1833:1855 | − | Transgenic |
| | | | | | insert |
| TI-119 | CTTC | FnCas12a | 1834:1856 | − | Transgenic |
| | | | | | insert |
| TI-120 | TTCG | LbCas12a-TYC | 1840:1862 | − | Transgenic |
| | | | | | insert |
| TI-121 | TTTC | LbCas12a | 1841:1863 | − | Transgenic |
| | | | | | insert |
| TI-122 | GTTC | FnCas12a | 1864:1886 | + | Transgenic |
| | | | | | insert |
| TI-123 | TTCG | LbCas12a-TYC | 1865:1887 | + | Transgenic |
| | | | | | insert |
| TI-124 | TCCC | LbCas12a-TYC | 1872:1894 | + | Transgenic |
| | | | | | insert |
| TI-125 | GTTG | FnCas12a | 1876:1898 | + | Transgenic |
| | | | | | insert |
| TI-126 | GTTG | FnCas12a | 1861:1883 | − | Transgenic |
| | | | | | insert |
| TI-127 | TCCA | LbCas12a-TYC | 1886:1908 | + | Transgenic |
| | | | | | insert |
| TI-128 | TCCG | LbCas12a-TYC | 1904:1926 | + | Transgenic |
| | | | | | insert |
| TI-129 | TTTG | LbCas12a | 1910:1932 | + | Transgenic |
| | | | | | insert |
| TI-130 | GTTC | FnCas12a | 1932:1954 | + | Transgenic |
| | | | | | insert |
| TI-131 | TTCC | LbCas12a-TYC | 1933:1955 | + | Transgenic |
| | | | | | insert |
| TI-132 | TCCG | LbCas12a-TYC | 1934:1956 | + | Transgenic |
| | | | | | insert |
| TI-133 | CTTC | FnCas12a | 1957:1979 | + | Transgenic |
| | | | | | insert |
| TI-134 | TTCA | LbCas12a-TYC | 1958:1980 | + | Transgenic |
| | | | | | insert |
| TI-135 | CTTC | FnCas12a | 1947:1969 | − | Transgenic |
| | | | | | insert |
| TI-136 | GTTA | FnCas12a | 1982:2004 | + | Transgenic |
| | | | | | insert |
| TI-137 | TCCG | LbCas12a-TYC | 1991:2013 | + | Transgenic |
| | | | | | insert |
| TI-138 | TTTA | LbCas12a | 1987:2009 | − | Transgenic |
| | | | | | insert |
| TI-139 | CTTA | FnCas12a | 2012:2034 | + | Transgenic |
| | | | | | insert |
| TI-140 | TATG | LbCas12a-TATV | 2003:2025 | − | Transgenic |
| | | | | | insert |
| TI-141 | TCCA | LbCas12a-TYC | 2028:2050 | + | Transgenic |
| | | | | | insert |
| TI-142 | TTTG | LbCas12a | 2036:2058 | + | Transgenic |
| | | | | | insert |
| TI-143 | CTTA | FnCas12a | 2015:2037 | − | Transgenic |
| | | | | | insert |
| TI-144 | TTTA | LbCas12a | 2070:2092 | − | Transgenic |
| | | | | | insert |
| TI-145 | TCCA | LbCas12a-TYC | 2077:2099 | − | Transgenic |
| | | | | | insert |
| TI-146 | TTCC | LbCas12a-TYC | 2108:2130 | + | Transgenic |
| | | | | | insert |
| TI-147 | TCCA | LbCas12a-TYC | 2109:2131 | + | Transgenic |
| | | | | | insert |
| TI-148 | TCCA | LbCas12a-TYC | 2116:2138 | + | Transgenic |
| | | | | | insert |
| TI-149 | CTTA | FnCas12a | 2097:2119 | − | Transgenic |
| | | | | | insert |
| TI-150 | TTTC | LbCas12a | 2130:2152 | + | Transgenic |
| | | | | | insert |
| TI-151 | TTCG | LbCas12a-TYC | 2131:2153 | + | Transgenic |
| | | | | | insert |
| TI-152 | CTTC | FnCas12a | 2138:2160 | + | Transgenic |
| | | | | | insert |
| TI-153 | TTCC | LbCas12a-TYC | 2139:2161 | + | Transgenic |
| | | | | | insert |
| TI-154 | TCCA | LbCas12a-TYC | 2140:2162 | + | Transgenic |
| | | | | | insert |
| TI-155 | TCCA | LbCas12a-TYC | 2147:2169 | + | Transgenic |
| | | | | | insert |
| TI-156 | TTCC | LbCas12a-TYC | 2161:2183 | + | Transgenic |
| | | | | | insert |
| TI-157 | TCCC | LbCas12a-TYC | 2162:2184 | + | Transgenic |
| | | | | | insert |
| TI-158 | CTTC | FnCas12a | 2176:2198 | + | Transgenic |
| | | | | | insert |
| TI-159 | TTCG | LbCas12a-TYC | 2177:2199 | + | Transgenic |
| | | | | | insert |
| TI-160 | TCCG | LbCas12a-TYC | 2171:2193 | − | Transgenic |
| | | | | | insert |
| TI-161 | TATC | LbCas12a-TATV | 2173:2195 | − | Transgenic |
| | | | | | insert |
| TI-162 | CTTG | FnCas12a | 2209:2231 | + | Transgenic |
| | | | | | insert |
| TI-163 | CTTG | FnCas12a | 2195:2217 | − | Transgenic |
| | | | | | insert |
| TI-164 | TCCA | LbCas12a-TYC | 2220:2242 | + | Transgenic |
| | | | | | insert |
| TI-165 | TCCA | LbCas12a-TYC | 2233:2255 | + | Transgenic |
| | | | | | insert |
| TI-166 | TTCC | LbCas12a-TYC | 2237:2259 | + | Transgenic |
| | | | | | insert |
| TI-167 | TTTG | LbCas12a | 2248:2270 | + | Transgenic |
| | | | | | insert |
| TI-168 | GTTC | FnCas12a | 2254:2276 | + | Transgenic |
| | | | | | insert |
| TI-169 | TTCG | LbCas12a-TYC | 2255:2277 | + | Transgenic |
| | | | | | insert |
| TI-170 | TTCG | LbCas12a-TYC | 2235:2257 | − | Transgenic |
| | | | | | insert |
| TI-171 | TTTC | LbCas12a | 2236:2258 | − | Transgenic |
| | | | | | insert |
| TI-172 | TTTA | LbCas12a | 2268:2290 | + | Transgenic |
| | | | | | insert |
| TI-173 | TCCA | LbCas12a-TYC | 2248:2270 | − | Transgenic |
| | | | | | insert |
| TI-174 | TTTA | LbCas12a | 2295:2317 | + | Transgenic |
| | | | | | insert |
| TI-175 | TATG | LbCas12a-TATV | 2280:2302 | − | Transgenic |
| | | | | | insert |
| TI-176 | GTTG | FnCas12a | 2331:2353 | + | Transgenic |
| | | | | | insert |
| TI-177 | TCCC | LbCas12a-TYC | 2336:2358 | − | Transgenic |
| | | | | | insert |
| TI-178 | CTTG | FnCas12a | 2355:2377 | − | Transgenic |
| | | | | | insert |
| TI-179 | TATG | LbCas12a-TATV | 2397:2419 | + | Transgenic |
| | | | | | insert |
| TI-180 | GTTG | FnCas12a | 2379:2401 | − | Transgenic |
| | | | | | insert |
| TI-181 | CTTG | FnCas12a | 2384:2406 | − | Transgenic |
| | | | | | insert |
| TI-182 | CTTC | FnCas12a | 2387:2409 | − | Transgenic |
| | | | | | insert |
| TI-183 | TTCA | LbCas12a-TYC | 2392:2414 | − | Transgenic |
| | | | | | insert |
| TI-184 | CTTC | FnCas12a | 2393:2415 | − | Transgenic |
| | | | | | insert |
| TI-185 | TTCC | LbCas12a-TYC | 2400:2422 | − | Transgenic |
| | | | | | insert |
| TI-186 | CTTC | FnCas12a | 2415:2437 | − | Transgenic |
| | | | | | insert |
| TI-187 | CTTC | FnCas12a | 2418:2440 | − | Transgenic |
| | | | | | insert |
| TI-188 | CTTC | FnCas12a | 2421:2443 | − | Transgenic |
| | | | | | insert |
| TI-189 | CTTA | FnCas12a | 2452:2474 | + | Transgenic |
| | | | | | insert |
| TI-190 | GTTG | FnCas12a | 2463:2485 | + | Transgenic |
| | | | | | insert |
| TI-191 | CTTG | FnCas12a | 2447:2469 | − | Transgenic |
| | | | | | insert |
| TI-192 | TTTC | LbCas12a | 2450:2472 | − | Transgenic |
| | | | | | insert |
| TI-193 | TATA | LbCas12a-TATV | 2464:2486 | − | Transgenic |
| | | | | | insert |
| TI-194 | TTTA | LbCas12a | 2466:2488 | − | Transgenic |
| | | | | | insert |
| TI-195 | TTTA | LbCas12a | 2489:2511 | + | Transgenic |
| | | | | | insert |
| TI-196 | TATA | LbCas12a-TATV | 2491:2513 | + | Transgenic |
| | | | | | insert |
| TI-197 | GTTG | FnCas12a | 2479:2501 | − | Transgenic |
| | | | | | insert |
| TI-198 | GTTG | FnCas12a | 2482:2504 | − | Transgenic |
| | | | | | insert |
| TI-199 | TTCG | LbCas12a-TYC | 2516:2538 | + | Transgenic |
| | | | | | insert |
| TI-200 | GTTA | FnCas12a | 2519:2541 | + | Transgenic |
| | | | | | insert |
| TI-201 | TATC | LbCas12a-TATV | 2521:2543 | + | Transgenic |
| | | | | | insert |
| TI-202 | CTTC | FnCas12a | 2526:2548 | + | Transgenic |
| | | | | | insert |
| TI-203 | TTCG | LbCas12a-TYC | 2527:2549 | + | Transgenic |
| | | | | | insert |
| TI-204 | CTTG | FnCas12a | 2508:2530 | − | Transgenic |
| | | | | | insert |
| TI-205 | CTTC | FnCas12a | 2532:2554 | + | Transgenic |
| | | | | | insert |
| TI-206 | TTCA | LbCas12a-TYC | 2533:2555 | + | Transgenic |
| | | | | | insert |
| TI-207 | GTTA | FnCas12a | 2515:2537 | − | Transgenic |
| | | | | | insert |
| TI-208 | CTTA | FnCas12a | 2540:2562 | + | Transgenic |
| | | | | | insert |
| TI-209 | TCCG | LbCas12a-TYC | 2539:2561 | − | Transgenic |
| | | | | | insert |
| TI-210 | CTTG | FnCas12a | 2548:2570 | − | Transgenic |
| | | | | | insert |
| TI-211 | GTTG | FnCas12a | 2578:2600 | + | Transgenic |
| | | | | | insert |
| TI-212 | TTCC | LbCas12a-TYC | 2561:2583 | − | Transgenic |
| | | | | | insert |
| TI-213 | CTTC | FnCas12a | 2562:2584 | − | Transgenic |
| | | | | | insert |
| TI-214 | TTTG | LbCas12a | 2612:2634 | + | Transgenic |
| | | | | | insert |
| TI-215 | TCCA | LbCas12a-TYC | 2621:2643 | + | Transgenic |
| | | | | | insert |
| TI-216 | TCCA | LbCas12a-TYC | 2600:2622 | − | Transgenic |
| | | | | | insert |
| TI-217 | TTCC | LbCas12a-TYC | 2601:2623 | − | Transgenic |
| | | | | | insert |
| TI-218 | TATG | LbCas12a-TATV | 2632:2654 | + | Transgenic |
| | | | | | insert |
| TI-219 | TTTA | LbCas12a | 2636:2658 | + | Transgenic |
| | | | | | insert |
| TI-220 | TATG | LbCas12a-TATV | 2638:2660 | + | Transgenic |
| | | | | | insert |
| TI-221 | GTTG | FnCas12a | 2644:2666 | + | Transgenic |
| | | | | | insert |
| TI-222 | TTTG | LbCas12a | 2625:2647 | − | Transgenic |
| | | | | | insert |
| TI-223 | TCCA | LbCas12a-TYC | 2646:2668 | − | Transgenic |
| | | | | | insert |
| TI-224 | TTTC | LbCas12a | 2688:2710 | + | Transgenic |
| | | | | | insert |
| TI-225 | TTCA | LbCas12a-TYC | 2689:2711 | + | Transgenic |
| | | | | | insert |
| TI-226 | TATA | LbCas12a-TATV | 2702:2724 | + | Transgenic |
| | | | | | insert |
| TI-227 | TTTA | LbCas12a | 2712:2734 | + | Transgenic |
| | | | | | insert |
| TI-228 | TATG | LbCas12a-TATV | 2695:2717 | − | Transgenic |
| | | | | | insert |
| TI-229 | TCCA | LbCas12a-TYC | 2704:2726 | − | Transgenic |
| | | | | | insert |
| TI-230 | TTCC | LbCas12a-TYC | 2705:2727 | − | Transgenic |
| | | | | | insert |
| TI-231 | TTTC | LbCas12a | 2706:2728 | − | Transgenic |
| | | | | | insert |
| TI-232 | TTTG | LbCas12a | 2756:2778 | + | Transgenic |
| | | | | | insert |
| TI-233 | TATG | LbCas12a-TATV | 2752:2774 | − | Transgenic |
| | | | | | insert |
| TI-234 | TTCA | LbCas12a-TYC | 2777:2799 | + | Transgenic |
| | | | | | insert |
| TI-235 | TATG | LbCas12a-TATV | 2781:2803 | + | Transgenic |
| | | | | | insert |
| TI-236 | CTTG | FnCas12a | 2762:2784 | − | Transgenic |
| | | | | | insert |
| TI-237 | TATC | LbCas12a-TATV | 2765:2787 | − | Transgenic |
| | | | | | insert |
| TI-238 | CTTC | FnCas12a | 2796:2818 | + | Transgenic |
| | | | | | insert |
| TI-239 | CTTC | FnCas12a | 2806:2828 | + | Transgenic |
| | | | | | insert |
| TI-240 | TATG | LbCas12a-TATV | 2785:2807 | − | Transgenic |
| | | | | | insert |
| TI-241 | CTTC | FnCas12a | 2809:2831 | + | Transgenic |
| | | | | | insert |
| TI-242 | TTCA | LbCas12a-TYC | 2810:2832 | + | Transgenic |
| | | | | | insert |
| TI-243 | TATC | LbCas12a-TATV | 2790:2812 | − | Transgenic |
| | | | | | insert |
| TI-244 | GTTA | FnCas12a | 2792:2814 | − | Transgenic |
| | | | | | insert |
| TI-245 | TTTG | LbCas12a | 2800:2822 | − | Transgenic |
| | | | | | insert |
| TI-246 | TTCC | LbCas12a-TYC | 2824:2846 | + | Transgenic |
| | | | | | insert |
| TI-247 | TCCA | LbCas12a-TYC | 2825:2847 | + | Transgenic |
| | | | | | insert |
| TI-248 | TCCA | LbCas12a-TYC | 2808:2830 | − | Transgenic |
| | | | | | insert |
| TI-249 | TATC | LbCas12a-TATV | 2810:2832 | − | Transgenic |
| | | | | | insert |
| TI-250 | TTTG | LbCas12a | 2833:2855 | + | Transgenic |
| | | | | | insert |
| TI-251 | CTTA | FnCas12a | 2850:2872 | + | Transgenic |
| | | | | | insert |
| TI-252 | TATC | LbCas12a-TATV | 2829:2851 | − | Transgenic |
| | | | | | insert |
| TI-253 | TATC | LbCas12a-TATV | 2852:2874 | + | Transgenic |
| | | | | | insert |
| TI-254 | TTTG | LbCas12a | 2842:2864 | − | Transgenic |
| | | | | | insert |
| TI-255 | GTTC | FnCas12a | 2865:2887 | + | Transgenic |
| | | | | | insert |
| TI-256 | TTCC | LbCas12a-TYC | 2866:2888 | + | Transgenic |
| | | | | | insert |
| TI-257 | TCCA | LbCas12a-TYC | 2867:2889 | + | Transgenic |
| | | | | | insert |
| TI-258 | GTTA | FnCas12a | 2846:2868 | − | Transgenic |
| | | | | | insert |
| TI-259 | TTTA | LbCas12a | 2855:2877 | − | Transgenic |
| | | | | | insert |
| TI-260 | TCCA | LbCas12a-TYC | 2867:2889 | − | Transgenic |
| | | | | | insert |
| TI-261 | TTTC | LbCas12a | 2873:2895 | − | Transgenic |
| | | | | | insert |
| TI-262 | CTTA | FnCas12a | 2908:2930 | + | Transgenic |
| | | | | | insert |
| TI-263 | TATA | LbCas12a-TATV | 2895:2917 | − | Transgenic |
| | | | | | insert |
| TI-264 | TTTG | LbCas12a | 2934:2956 | + | Transgenic |
| | | | | | insert |
| TI-265 | GTTA | FnCas12a | 2937:2959 | + | Transgenic |
| | | | | | insert |
| TI-266 | TATA | LbCas12a-TATV | 2939:2961 | + | Transgenic |
| | | | | | insert |
| TI-267 | TATA | LbCas12a-TATV | 2941:2963 | + | Transgenic |
| | | | | | insert |
| TI-268 | TATC | LbCas12a-TATV | 2926:2948 | − | Transgenic |
| | | | | | insert |
| TI-269 | GTTG | FnCas12a | 2930:2952 | − | Transgenic |
| | | | | | insert |
| TI-270 | CTTG | FnCas12a | 2940:2962 | − | Transgenic |
| | | | | | insert |
| TI-271 | TATC | LbCas12a-TATV | 2964:2986 | + | Transgenic |
| | | | | | insert |
| TI-272 | TATG | LbCas12a-TATV | 2971:2993 | + | Transgenic |
| | | | | | insert |
| TI-273 | TTTG | LbCas12a | 2955:2977 | − | Transgenic |
| | | | | | insert |
| TI-274 | GTTA | FnCas12a | 2977:2999 | + | Transgenic |
| | | | | | insert |
| TI-275 | TATC | LbCas12a-TATV | 2979:3001 | + | Transgenic |
| | | | | | insert |
| TI-276 | TATG | LbCas12a-TATV | 2964:2986 | − | Transgenic |
| | | | | | insert |
| TI-277 | TTTA | LbCas12a-TYC | 2974:2996 | − | Transgenic |
| | | | | | insert |
| TI-278 | TTTA | LbCas12a | 2982:3004 | − | Transgenic |
| | | | | | insert |
| TI-279 | CTTA | FnCas12a | 3047:3069 | − | Transgenic |
| | | | | | insert |
| TI-280 | TATC | LbCas12a-TATV | 3055:3077 | − | Transgenic |
| | | | | | insert |
| TI-281 | TTTG | LbCas12a | 3079:3101 | + | Transgenic |
| | | | | | insert |
| TI-282 | TTTA | LbCas12a | 3057:3079 | − | Transgenic |
| | | | | | insert |
| TI-283 | TATA | LbCas12a-TATV | 3061:3083 | − | Transgenic |
| | | | | | insert |
| TI-284 | TATA | LbCas12a-TATV | 3063:3085 | − | Transgenic |
| | | | | | insert |
| TI-285 | TATA | LbCas12a-TATV | 3088:3110 | + | Transgenic |
| | | | | | insert |
| TI-286 | TATA | LbCas12a-TATV | 3090:3112 | + | Transgenic |
| | | | | | insert |
| TI-287 | TTTA | LbCas12a | 3095:3117 | + | Transgenic |
| | | | | | insert |
| TI-288 | TTCA | LbCas12a-TYC | 3077:3099 | − | Transgenic |
| | | | | | insert |
| TI-289 | TTTA | LbCas12a | 3082:3104 | − | Transgenic |
| | | | | | insert |
| TI-290 | TATC | LbCas12a-TATV | 3086:3108 | − | Transgenic |
| | | | | | insert |
| TI-291 | CTTA | FnCas12a | 3098:3120 | − | Transgenic |
| | | | | | insert |
| TI-292 | TATC | LbCas12a-TATV | 3101:3123 | − | Transgenic |
| | | | | | insert |
| TI-293 | TTTG | LbCas12a | 3113:3135 | − | Transgenic |
| | | | | | insert |
| TI-294 | TATG | LbCas12a-TATV | 3121:3143 | − | Transgenic |
| | | | | | insert |
| TI-295 | TTTA | LbCas12a | 3128:3150 | − | Transgenic |
| | | | | | insert |
| TI-296 | TTCA | LbCas12a-TYC | 3135:3157 | − | Transgenic |
| | | | | | insert |
| TI-297 | TTTA | LbCas12a | 3155:3177 | − | Transgenic |
| | | | | | insert |
| TI-298 | TTTA | FnCas12a | 3180:3202 | + | Transgenic |
| | | | | | insert |
| TI-299 | TTCA | LbCas12a-TYC | 3186:3208 | + | Transgenic |
| | | | | | insert |
| TI-300 | TTCA | LbCas12a-TYC | 3166:3188 | − | Transgenic |
| | | | | | insert |
| TI-301 | TTTC | FnCas12a | 3167:3189 | − | Transgenic |
| | | | | | insert |
| TI-302 | TTCA | LbCas12a-TYC | 3174:3196 | − | Transgenic |
| | | | | | insert |
| TI-303 | TATG | LbCas12a-TATV | 3199:3221 | + | Transgenic |
| | | | | | insert |
| TI-304 | TATA | LbCas12a-TATV | 3205:3227 | + | Transgenic |
| | | | | | insert |
| TI-305 | TTTC | LbCas12a | 3185:3207 | − | Transgenic |
| | | | | | insert |
| TI-306 | TATC | LbCas12a-TATV | 3207:3229 | + | Transgenic |
| | | | | | insert |
| TI-307 | TTCA | LbCas12a-TYC | 3195:3217 | − | Transgenic |
| | | | | | insert |
| TI-308 | TTTA | LbCas12a-TYC | 3210:3232 | − | Transgenic |
| | | | | | insert |
| TI-309 | TATA | LbCas12a-TATV | 3255:3277 | + | Transgenic |
| | | | | | insert |
| TI-310 | TATC | LbCas12a-TATV | 3257:3279 | + | Transgenic |
| | | | | | insert |
| TI-311 | TTTA | LbCas12a | 3265:3287 | + | Transgenic |
| | | | | | insert |
| TI-312 | TTTG | LbCas12a | 3251:3273 | − | Transgenic |
| | | | | | insert |
| TI-313 | TATA | LbCas12a-TATV | 3260:3282 | − | Transgenic |
| | | | | | insert |
| TI-314 | TTTA | LbCas12a | 3262:3284 | − | Transgenic |
| | | | | | insert |
| TI-315 | TATA | LbCas12a-TATV | 3310:3332 | + | Transgenic |
| | | | | | insert |
| TI-316 | TATA | LbCas12a-TATV | 3312:3334 | + | Transgenic |
| | | | | | insert |
| TI-317 | TTCA | LbCas12a-TYC | 3316:3338 | + | Transgenic |
| | | | | | insert |
| TI-318 | CTTG | FnCas12a | 3299:3321 | − | Transgenic |
| | | | | | insert |
| TI-319 | TTTC | LbCas12a | 3337:3359 | + | Transgenic |
| | | | | | insert |
| TI-320 | CTTA | FnCas12a | 3319:3341 | − | Transgenic |
| | | | | | insert |
| TI-321 | TATA | LbCas12a-TATV | 3324:3346 | − | Transgenic |
| | | | | | insert |
| TI-322 | TTTA | LbCas12a | 3326:3348 | − | Transgenic |
| | | | | | insert |
| TI-323 | TATA | LbCas12a-TATV | 3351:3373 | + | Transgenic |
| | | | | | insert |
| TI-324 | TTCA | LbCas12a-TYC | 3331:3353 | − | Transgenic |
| | | | | | insert |
| TI-325 | TTTC | LbCas12a | 3332:3354 | − | Transgenic |
| | | | | | insert |
| TI-326 | CTTC | FnCas12a | 3365:3387 | + | Transgenic |
| | | | | | insert |
| TI-327 | TTCA | LbCas12a-TYC | 3366:3388 | + | Transgenic |
| | | | | | insert |
| TI-328 | CTTG | FnCas12a | 3370:3392 | + | Transgenic |
| | | | | | insert |
| TI-329 | TATA | LbCas12a-TATV | 3356:3378 | − | Transgenic |
| | | | | | insert |
| TI-330 | TTTC | LbCas12a | 3388:3410 | + | Transgenic |
| | | | | | insert |
| TI-331 | CTTC | FnCas12a | 3399:3421 | + | Transgenic |
| | | | | | insert |
| TI-332 | TTCA | LbCas12a-TYC | 3400:3422 | + | Transgenic |
| | | | | | insert |
| TI-333 | TTTC | LbCas12a | 3408:3430 | + | Transgenic |
| | | | | | insert |
| TI-334 | TTCA | LbCas12a-TYC | 3409:3431 | + | Transgenic |
| | | | | | insert |
| TI-335 | TTTC | LbCas12a | 3415:3437 | + | Transgenic |
| | | | | | insert |
| TI-336 | GTTG | FnCas12a | 3393:3415 | − | Transgenic |
| | | | | | insert |
| TI-337 | TCCA | LbCas12a-TYC | 3409:3431 | − | Transgenic |
| | | | | | insert |
| TI-338 | TTCC | LbCas12a-TYC | 3410:3432 | − | Transgenic |
| | | | | | insert |
| TI-339 | GTTC | FnCas12a | 3411:3433 | − | Transgenic |
| | | | | | insert |
| TI-340 | TATG | LbCas12a-TATV | 3422:3444 | − | Transgenic |
| | | | | | insert |
| TI-341 | TATC | LbCas12a-TATV | 3451:3473 | + | Transgenic |
| | | | | | insert |
| TI-342 | TCCA | LbCas12a-TYC | 3433:3455 | − | Transgenic |
| | | | | | insert |
| TI-343 | TTTG | LbCas12a | 3437:3459 | − | Transgenic |
| | | | | | insert |
| TI-344 | TTTG | LbCas12a | 3443:3465 | − | Transgenic |
| | | | | | insert |
| TI-345 | TTTA | LbCas12a | 3475:3497 | + | Transgenic |
| | | | | | insert |
| TI-346 | TTTA | LbCas12a | 3481:3503 | + | Transgenic |
| | | | | | insert |
| TI-347 | CTTC | FnCas12a | 3487:3509 | + | Transgenic |
| | | | | | insert |
| TI-348 | TTCA | LbCas12a-TYC | 3488:3510 | + | Transgenic |
| | | | | | insert |
| TI-349 | TTCG | LbCas12a-TYC | 3492:3514 | + | Transgenic |
| | | | | | insert |
| TI-350 | TTCG | LbCas12a-TYC | 3476:3498 | − | Transgenic |
| | | | | | insert |
| TI-351 | CTTC | FnCas12a | 3477:3499 | − | Transgenic |
| | | | | | insert |
| TI-352 | TATG | LbCas12a-TATV | 3511:3533 | + | Transgenic |
| | | | | | insert |
| TI-353 | GTTC | FnCas12a | 3520:3542 | + | Transgenic |
| | | | | | insert |
| TI-354 | CTTG | FnCas12a | 3503:3525 | − | Transgenic |
| | | | | | insert |
| TI-355 | CTTG | FnCas12a | 3508:3530 | − | Transgenic |
| | | | | | insert |
| TI-356 | GTTC | FnCas12a | 3513:3535 | − | Transgenic |
| | | | | | insert |
| TI-357 | TTTG | LbCas12a | 3516:3538 | − | Transgenic |
| | | | | | insert |
| TI-358 | TATC | LbCas12a-TATV | 3527:3549 | − | Transgenic |
| | | | | | insert |
| TI-359 | TATC | LbCas12a-TATV | 3532:3554 | − | Transgenic |
| | | | | | insert |
| TI-360 | TATG | LbCas12a-TATV | 3556:3578 | + | Transgenic |
| | | | | | insert |
| TI-361 | TTTA | LbCas12a | 3537:3559 | − | Transgenic |
| | | | | | insert |
| TI-362 | TATG | LbCas12a-TATV | 3553:3575 | − | Transgenic |
| | | | | | insert |
| TI-363 | TATA | LbCas12a-TATV | 3555:3577 | − | Transgenic |
| | | | | | insert |
| TI-364 | TCCA | LbCas12a-TYC | 3578:3600 | + | Transgenic |
| | | | | | insert |
| TI-365 | TATA | LbCas12a-TATV | 3557:3579 | − | Transgenic |
| | | | | | insert |
| TI-366 | CTTA | FnCas12a | 3559:3581 | − | Transgenic |
| | | | | | insert |
| TI-367 | TATA | LbCas12a-TATV | 3582:3604 | + | Transgenic |
| | | | | | insert |
| TI-368 | TATA | LbCas12a-TATV | 3584:3606 | + | Transgenic |
| | | | | | insert |
| TI-369 | TTCA | LbCas12a-TYC | 3569:3591 | − | Transgenic |
| | | | | | insert |
| TI-370 | CTTC | FnCas12a | 3570:3592 | − | Transgenic |
| | | | | | insert |
| TI-371 | GTTG | FnCas12a | 3594:3616 | + | Transgenic |
| | | | | | insert |
| TI-372 | GTTC | FnCas12a | 3573:3595 | − | Transgenic |
| | | | | | insert |
| TI-373 | TTTG | LbCas12a | 3577:3599 | − | Transgenic |
| | | | | | insert |
| TI-374 | TATC | LbCas12a-TATV | 3586:3608 | − | Transgenic |
| | | | | | insert |
| TI-375 | TCCA | LbCas12a-TYC | 3599:3621 | − | Transgenic |
| | | | | | insert |
| TI-376 | TTTA | LbCas12a | 3619:3641 | − | Transgenic |
| | | | | | insert |
| TI-377 | GTTA | FnCas12a | 3626:3648 | − | Transgenic |
| | | | | | insert |
| TI-378 | GTTA | FnCas12a | 3651:3673 | + | Transgenic |
| | | | | | insert |
| TI-379 | TATG | LbCas12a-TATV | 3659:3681 | + | Transgenic |
| | | | | | insert |
| TI-380 | TTTG | LbCas12a | 3639:3661 | − | Transgenic |
| | | | | | insert |
| TI-381 | TTTG | LbCas12a | 3648:3670 | − | Transgenic |
| | | | | | insert |
| TI-382 | TTTG | LbCas12a | 3681:3703 | + | Transgenic |
| | | | | | insert |
| TI-383 | TTTG | LbCas12a | 3694:3716 | + | Transgenic |
| | | | | | insert |
| TI-384 | CTTA | FnCas12a | 3701:3723 | + | Transgenic |
| | | | | | insert |
| TI-385 | TATG | LbCas12a-TATV | 3683:3705 | − | Transgenic |
| | | | | | insert |
| TI-386 | GTTA | FnCas12a | 3696:3718 | − | Transgenic |
| | | | | | insert |
| TI-387 | TTTA | LbCas12a | 3721:3743 | + | Transgenic |
| | | | | | insert |
| TI-388 | TATG | LbCas12a-TATV | 3699:3721 | − | Transgenic |
| | | | | | insert |
| TI-389 | TTTC | LbCas12a | 3712:3734 | − | Transgenic |
| | | | | | insert |
| TI-390 | GTTA | FnCas12a | 3735:3757 | + | Transgenic |
| | | | | | insert |
| TI-391 | TTCA | LbCas12a-TYC | 3717:3739 | − | Transgenic |
| | | | | | insert |
| TI-392 | CTTC | FnCas12a | 3718:3740 | − | Transgenic |
| | | | | | insert |
| TI-393 | TTTA | LbCas12a | 3727:3749 | − | Transgenic |
| | | | | | insert |
| TI-394 | TATA | LbCas12a-TATV | 3736:3758 | − | Transgenic |
| | | | | | insert |
| TI-395 | TTTA | LbCas12a | 3738:3760 | − | Transgenic |
| | | | | | insert |
| TI-396 | TTTA | LbCas12a | 3761:3783 | + | Transgenic |
| | | | | | insert |
| TI-397 | TATA | LbCas12a-TATV | 3763:3785 | + | Transgenic |
| | | | | | insert |
| TI-398 | TCCC | LbCas12a-TYC | 3753:3775 | − | Transgenic |
| | | | | | insert |
| TI-399 | GTTG | FnCas12a | 3777:3799 | + | Transgenic |
| | | | | | insert |
| TI-400 | TTTG | LbCas12a | 3760:3782 | − | Transgenic |
| | | | | | insert |
| TI-401 | TTTA | LbCas12a | 3776:3798 | − | Transgenic |
| | | | | | insert |
| TI-402 | TTTA | LbCas12a | 3838:3860 | + | Transgenic |
| | | | | | insert |
| TI-403 | TATA | LbCas12a-TATV | 3844:3866 | + | Transgenic |
| | | | | | insert |
| TI-404 | GTTA | FnCas12a | 3841:3863 | − | Transgenic |
| | | | | | insert |
| TI-405 | TATA | LbCas12a-TATV | 3903:3925 | + | Transgenic |
| | | | | | insert |
| TI-406 | TATC | LbCas12a-TATV | 3912:3934 | + | Transgenic |
| | | | | | insert |
| TI-407 | TTTA | LbCas12a | 3893:3915 | − | Transgenic |
| | | | | | insert |
| TI-408 | TTTC | LbCas12a | 3916:3938 | + | Transgenic |
| | | | | | insert |
| TI-409 | TTTG | LbCas12a | 3911:3933 | − | Transgenic |
| | | | | | insert |
| TI-410 | TATA | LbCas12a-TATV | 3944:3966 | + | Transgenic |
| | | | | | insert |
| TI-411 | TATC | LbCas12a-TATV | 3946:3968 | + | Transgenic |
| | | | | | insert |
| TI-412 | TTTC | LbCas12a | 3930:3952 | − | Transgenic |
| | | | | | insert |
| TI-413 | TTTG | LbCas12a | 3941:3963 | − | Transgenic |
| | | | | | insert |
| TI-414 | TTTG | LbCas12a | 3977:3999 | + | Transgenic |
| | | | | | insert |
| TI-415 | TTCG | LbCas12a-TYC | 3982:4004 | − | Transgenic |
| | | | | | insert |
| TI-416 | CTTC | FnCas12a | 3983:4005 | − | Transgenic |
| | | | | | insert |
| TI-417 | CTTG | FnCas12a | 3996:4018 | − | Transgenic |
| | | | | | insert |
| TI-418 | TTTA | LbCas12a | 4019:4041 | + | Transgenic |
| | | | | | insert |
| TI-419 | TATC | LbCas12a-TATV | 4039:4061 | + | Transgenic |
| | | | | | insert |
| TI-420 | TTCA | LbCas12a-TYC | 4018:4040 | − | Transgenic |
| | | | | | insert |
| TI-421 | TTTC | LbCas12a | 4019:4041 | − | Transgenic |
| | | | | | insert |
| TI-422 | CTTG | FnCas12a | 4029:4051 | − | Transgenic |
| | | | | | insert |
| TI-423 | GTTC | FnCas12a | 4055:4077 | − | Transgenic |
| | | | | | insert |
| TI-424 | TTTG | LbCas12a | 4092:4114 | + | Transgenic |
| | | | | | insert |
| TI-425 | TTCA | LbCas12a-TYC | 4078:4100 | − | Transgenic |
| | | | | | insert |
| TI-426 | CTTC | FnCas12a | 4079:4101 | − | Transgenic |
| | | | | | insert |
| TI-427 | CTTG | FnCas12a | 4099:4121 | − | Transgenic |
| | | | | | insert |
| TI-428 | TCCA | LbCas12a-TYC | 4122:4144 | + | Transgenic |
| | | | | | insert |
| TI-429 | TTTC | LbCas12a | 4112:4134 | − | Transgenic |
| | | | | | insert |
| TI-430 | TTTC | LbCas12a | 4136:4158 | − | Transgenic |
| | | | | | insert |
| TI-431 | GTTG | FnCas12a | 4162:4184 | − | Transgenic |
| | | | | | insert |
| TI-432 | TCCA | LbCas12a-TYC | 4187:4209 | + | Transgenic |
| | | | | | insert |
| TI-433 | GTTA | FnCas12a | 4205:4227 | + | Transgenic |
| | | | | | insert |
| TI-434 | TATG | LbCas12a-TATV | 4207:4229 | + | Transgenic |
| | | | | | insert |
| TI-435 | TTCA | LbCas12a-TYC | 4191:4213 | − | Transgenic |
| | | | | | insert |
| TI-436 | CTTC | FnCas12a | 4192:4214 | − | Transgenic |
| | | | | | insert |
| TI-437 | TATC | LbCas12a-TATV | 4195:4217 | − | Transgenic |
| | | | | | insert |
| TI-438 | CTTA | FnCas12a | 4197:4219 | − | Transgenic |
| | | | | | insert |
| TI-439 | TTTA | LbCas12a | 4220:4242 | − | Transgenic |
| | | | | | insert |
| TI-440 | CTTC | FnCas12a | 4224:4246 | − | Transgenic |
| | | | | | insert |
| TI-441 | TTTC | LbCas12a | 4227:4249 | − | Transgenic |
| | | | | | insert |
| TI-442 | CTTG | FnCas12a | 4236:4258 | − | Transgenic |
| | | | | | insert |
| TI-443 | TATG | LbCas12a-TATV | 4297:4319 | + | Transgenic |
| | | | | | insert |
| TI-444 | TTTC | LbCas12a | 4301:4323 | + | Transgenic |
| | | | | | insert |
| TI-445 | TTCA | LbCas12a-TYC | 4302:4324 | + | Transgenic |
| | | | | | insert |
| TI-446 | TTTA | LbCas12a | 4331:4353 | + | Transgenic |
| | | | | | insert |
| TI-447 | TATC | LbCas12a-TATV | 4333:4355 | + | Transgenic |
| | | | | | insert |
| TI-448 | TTTA | LbCas12a | 4313:4335 | − | Transgenic |
| | | | | | insert |
| TI-449 | TATG | LbCas12a-TATV | 4346:4368 | + | Transgenic |
| | | | | | insert |
| TI-450 | TTTG | LbCas12a | 4350:4372 | + | Transgenic |
| | | | | | insert |
| TI-451 | CTTC | FnCas12a | 4356:4378 | + | Transgenic |
| | | | | | insert |
| TI-452 | TTCA | LbCas12a-TYC | 4357:4379 | + | Transgenic |
| | | | | | insert |
| TI-453 | TCCA | LbCas12a-TYC | 4337:4359 | − | Transgenic |
| | | | | | insert |
| TI-454 | TTCC | LbCas12a-TYC | 4338:4360 | − | Transgenic |
| | | | | | insert |
| TI-455 | GTTG | FnCas12a | 4362:4384 | + | Transgenic |
| | | | | | insert |
| TI-456 | TCCA | LbCas12a-TYC | 4342:4364 | − | Transgenic |
| | | | | | insert |
| TI-457 | TTCA | LbCas12a-TYC | 4353:4375 | − | Transgenic |
| | | | | | insert |
| TI-458 | TCCC | LbCas12a-TYC | 4364:4386 | − | Transgenic |
| | | | | | insert |
| TI-459 | GTTG | FnCas12a | 4387:4409 | + | Transgenic |
| | | | | | insert |
| TI-460 | TTTA | LbCas12a | 4369:4391 | − | Transgenic |
| | | | | | insert |
| TI-461 | TTCC | LbCas12a-TYC | 4402:4424 | + | Transgenic |
| | | | | | insert |
| TI-462 | TCCA | LbCas12a-TYC | 4403:4425 | + | Transgenic |
| | | | | | insert |
| TI-463 | GTTG | FnCas12a | 4382:4404 | − | Transgenic |
| | | | | | insert |
| TI-464 | CTTG | FnCas12a | 4385:4407 | − | Transgenic |
| | | | | | insert |
| TI-465 | TTCA | LbCas12a-TYC | 4407:4429 | + | Transgenic |
| | | | | | insert |
| TI-466 | GTTC | FnCas12a | 4415:4437 | + | Transgenic |
| | | | | | insert |
| TI-467 | TTCA | LbCas12a-TYC | 4416:4438 | + | Transgenic |
| | | | | | insert |
| TI-468 | TTTG | LbCas12a-TYC | 4397:4419 | − | Transgenic |
| | | | | | insert |
| TI-469 | TTTC | LbCas12a | 4421:4443 | + | Transgenic |
| | | | | | insert |
| TI-470 | TTCA | LbCas12a-TYC | 4422:4444 | + | Transgenic |
| | | | | | insert |
| TI-471 | CTTA | FnCas12a | 4409:4431 | − | Transgenic |
| | | | | | insert |
| TI-472 | TTCA | LbCas12a-TYC | 4416:4438 | − | Transgenic |
| | | | | | insert |
| TI-473 | TTTG | LbCas12a | 4479:4501 | + | Transgenic |
| | | | | | insert |
| TI-474 | GTTC | FnCas12a | 4482:4504 | + | Transgenic |
| | | | | | insert |
| TI-475 | TTCC | LbCas12a-TYC | 4483:4505 | + | Transgenic |
| | | | | | insert |
| TI-476 | CTTG | FnCas12a | 4486:4508 | + | Transgenic |
| | | | | | insert |
| TI-477 | TTTG | LbCas12a | 4490:4512 | + | Transgenic |
| | | | | | insert |
| TI-478 | CTTA | FnCas12a | 4505:4527 | + | Transgenic |
| | | | | | insert |
| TI-479 | TATC | LbCas12a-TATV | 4485:4507 | − | Transgenic |
| | | | | | insert |
| TI-480 | TATA | LbCas12a-TATV | 4487:4509 | − | Transgenic |
| | | | | | insert |
| TI-481 | TTTA | LbCas12a | 4494:4516 | − | Transgenic |
| | | | | | insert |
| TI-482 | TTTA | LbCas12a | 4624:4646 | + | Transgenic |
| | | | | | insert |
| TI-483 | TATA | LbCas12a-TATV | 4626:4648 | + | Transgenic |
| | | | | | insert |
| TI-484 | TTTA | LbCas12a | 4624:4646 | − | Transgenic |
| | | | | | insert |
| TI-485 | TTTC | LbCas12a | 4692:4714 | + | Transgenic |
| | | | | | insert |
| TI-486 | TTCA | LbCas12a-TYC | 4693:4715 | + | Transgenic |
| | | | | | insert |
| TI-487 | TTTA | LbCas12a | 4701:4723 | + | Transgenic |
| | | | | | insert |
| TI-488 | TTCA | LbCas12a-TYC | 4692:4714 | − | Transgenic |
| | | | | | insert |
| TI-489 | TTTC | LbCas12a | 4693:4715 | − | Transgenic |
| | | | | | insert |
| TI-490 | TTTG | LbCas12a-TYC | 4717:4739 | + | Transgenic |
| | | | | | insert |
| TI-491 | TTTA | LbCas12a | 4725:4747 | + | Transgenic |
| | | | | | insert |
| TI-492 | TATG | LbCas12a-TATV | 4727:4749 | + | Transgenic |
| | | | | | insert |
| TI-493 | CTTA | FnCas12a | 4711:4733 | − | Transgenic |
| | | | | | insert |
| TI-494 | TTTA | LbCas12a-TYC | 4733:4755 | + | Transgenic |
| | | | | | insert |
| TI-495 | GTTA | FnCas12a | 4722:4744 | − | Transgenic |
| | | | | | insert |
| TI-496 | TTTC | LbCas12a | 4745:4767 | + | Transgenic |
| | | | | | insert |
| TI-497 | TTTG | LbCas12a | 4727:4749 | − | Transgenic |
| | | | | | insert |
| TI-498 | CTTC | FnCas12a | 4762:4784 | + | Transgenic |
| | | | | | insert |
| TI-499 | TTCC | LbCas12a-TYC | 4763:4785 | + | Transgenic |
| | | | | | insert |
| TI-500 | TCCG | LbCas12a-TYC | 4764:4786 | + | Transgenic |
| | | | | | insert |
| TI-501 | TTTC | LbCas12a | 4774:4796 | + | Transgenic |
| | | | | | insert |
| TI-502 | TTCA | LbCas12a-TYC | 4775:4797 | + | Transgenic |
| | | | | | insert |
| TI-503 | CTTC | FnCas12a | 4767:4789 | − | Transgenic |
| | | | | | insert |
| TI-504 | TTTA | LbCas12a-TYC | 4804:4826 | + | Transgenic |
| | | | | | insert |
| TI-505 | TTTG | LbCas12a | 4819:4841 | + | Transgenic |
| | | | | | insert |
| TI-506 | TTTG | LbCas12a | 4829:4851 | + | Transgenic |
| | | | | | insert |
| TI-507 | TATG | LbCas12a-TATV | 4811:4833 | − | Transgenic |
| | | | | | insert |
| TI-508 | GTTC | FnCas12a | 4834:4856 | + | Transgenic |
| | | | | | insert |
| TI-509 | CTTA | FnCas12a | 4813:4835 | − | Transgenic |
| | | | | | insert |
| TI-510 | TTCC | LbCas12a-TYC | 4835:4857 | + | Transgenic |
| | | | | | insert |
| TI-511 | TCCA | LbCas12a-TYC | 4836:4858 | + | Transgenic |
| | | | | | insert |
| TI-512 | TTCA | LbCas12a-TYC | 4822:4844 | − | Transgenic |
| | | | | | insert |
| TI-513 | TTTC | LbCas12a | 4823:4845 | − | Transgenic |
| | | | | | insert |
| TI-514 | TATG | LbCas12a-TATV | 4860:4882 | + | Transgenic |
| | | | | | insert |
| TI-515 | CTTC | FnCas12a | 4873:4895 | + | Transgenic |
| | | | | | insert |
| TI-516 | TTCA | LbCas12a-TYC | 4874:4896 | + | Transgenic |
| | | | | | insert |
| TI-517 | TTTA | LbCas12a | 4866:4888 | − | Transgenic |
| | | | | | insert |
| TI-518 | CTTA | FnCas12a | 4891:4913 | + | Transgenic |
| | | | | | insert |
| TI-519 | CTTG | FnCas12a | 4872:4894 | − | Transgenic |
| | | | | | insert |
| TI-520 | TTTC | LbCas12a | 4875:4897 | − | Transgenic |
| | | | | | insert |
| TI-521 | TTCA | LbCas12a-TYC | 4897:4919 | + | Transgenic |
| | | | | | insert |
| TI-522 | TATC | LbCas12a-TATV | 4895:4917 | − | Transgenic |
| | | | | | insert |
| TI-523 | TTTG | LbCas12a | 4905:4927 | − | Transgenic |
| | | | | | insert |
| TI-524 | TTTC | LbCas12a | 4953:4975 | + | Transgenic |
| | | | | | insert |
| TI-525 | CTTC | FnCas12a | 4956:4978 | + | Transgenic |
| | | | | | insert |
| TI-526 | TTCC | LbCas12a-TYC | 4957:4979 | + | Transgenic |
| | | | | | insert |
| TI-527 | TCCC | LbCas12a-TYC | 4958:4980 | + | Transgenic |
| | | | | | insert |
| TI-528 | TTCA | LbCas12a-TYC | 4950:4972 | − | Transgenic |
| | | | | | insert |
| TI-529 | TCCA | LbCas12a-TYC | 4973:4995 | + | Transgenic |
| | | | | | insert |
| TI-530 | TTCA | LbCas12a-TYC | 4981:5003 | + | Transgenic |
| | | | | | insert |
| TI-531 | TTTG | LbCas12a | 4965:4987 | − | Transgenic |
| | | | | | insert |
| TI-532 | TTCC | LbCas12a-TYC | 5016:5038 | + | Transgenic |
| | | | | | insert |
| TI-533 | TATG | LbCas12a-TATV | 4997:5019 | − | Transgenic |
| | | | | | insert |
| TI-534 | TATA | LbCas12a-TATV | 4999:5021 | − | Transgenic |
| | | | | | insert |
| TI-535 | TATA | LbCas12a-TATV | 5001:5023 | − | Transgenic |
| | | | | | insert |
| TI-536 | TATA | LbCas12a-TATV | 5026:5048 | + | Transgenic |
| | | | | | insert |
| TI-537 | TATA | LbCas12a-TATV | 5028:5050 | + | Transgenic |
| | | | | | insert |
| TI-538 | CTTA | FnCas12a | 5033:5055 | + | Transgenic |
| | | | | | insert |
| TI-539 | TTTG | LbCas12a | 5018:5040 | − | Transgenic |
| | | | | | insert |
| TI-540 | TCCC | LbCas12a-TYC | 5040:5062 | + | Transgenic |
| | | | | | insert |
| TI-541 | TTTC | LbCas12a | 5022:5044 | − | Transgenic |
| | | | | | insert |
| TI-542 | TTCC | LbCas12a-TYC | 5031:5053 | − | Transgenic |
| | | | | | insert |
| TI-543 | CTTA | FnCas12a | 5054:5076 | + | Transgenic |
| | | | | | insert |
| TI-544 | CTTC | FnCas12a | 5032:5054 | − | Transgenic |
| | | | | | insert |
| TI-545 | TTTC | LbCas12a | 5070:5092 | + | Transgenic |
| | | | | | insert |
| TI-546 | TTTA | LbCas12a | 5076:5098 | + | Transgenic |
| | | | | | insert |
| TI-547 | CTTG | FnCas12a | 5055:5077 | − | Transgenic |
| | | | | | insert |
| TI-548 | TTCA | LbCas12a-TYC | 5080:5102 | + | Transgenic |
| | | | | | insert |
| TI-549 | TTTG | LbCas12a | 5070:5092 | − | Transgenic |
| | | | | | insert |
| TI-550 | TTTC | LbCas12a | 5075:5097 | − | Transgenic |
| | | | | | insert |
| TI-551 | TTTG | LbCas12a-TYC | 5101:5123 | − | Transgenic |
| | | | | | insert |
| TI-552 | TATC | LbCas12a-TATV | 5134:5156 | + | Transgenic |
| | | | | | insert |
| TI-553 | TTTG | LbCas12a | 5128:5150 | − | Transgenic |
| | | | | | insert |
| TI-554 | TTTA | LbCas12a | 5136:5158 | − | Transgenic |
| | | | | | insert |
| TI-555 | TTTC | LbCas12a | 5168:5190 | + | Transgenic |
| | | | | | insert |
| TI-556 | TTCA | LbCas12a-TYC | 5169:5191 | + | Transgenic |
| | | | | | insert |
| TI-557 | TATG | LbCas12a-TATV | 5151:5173 | − | Transgenic |
| | | | | | insert |
| TI-558 | TTTA | LbCas12a | 5153:5175 | − | Transgenic |
| | | | | | insert |
| TI-559 | TTCA | LbCas12a-TYC | 5157:5179 | − | Transgenic |
| | | | | | insert |
| TI-560 | TTTC | LbCas12a-TYC | 5158:5180 | − | Transgenic |
| | | | | | insert |
| TI-561 | TTCA | LbCas12a-TYC | 5170:5192 | − | Transgenic |
| | | | | | insert |
| TI-562 | TTTC | LbCas12a | 5171:5193 | − | Transgenic |
| | | | | | insert |
| TI-563 | TATC | LbCas12a-TATV | 5213:5235 | + | Transgenic |
| | | | | | insert |
| TI-564 | GTTA | FnCas12a | 5222:5244 | + | Transgenic |
| | | | | | insert |
| TI-565 | TTTC | LbCas12a | 5228:5250 | + | Transgenic |
| | | | | | insert |
| TI-566 | TTCA | LbCas12a-TYC | 5229:5251 | + | Transgenic |
| | | | | | insert |
| TI-567 | TTCA | LbCas12a-TYC | 5215:5237 | − | Transgenic |
| | | | | | insert |
| TI-568 | TATG | LbCas12a-TATV | 5240:5262 | + | Transgenic |
| | | | | | insert |
| TI-569 | TTCA | LbCas12a-TYC | 5221:5243 | − | Transgenic |
| | | | | | insert |
| TI-570 | CTTC | FnCas12a | 5222:5244 | − | Transgenic |
| | | | | | insert |
| TI-571 | TTTG | LbCas12a | 5255:5277 | + | Transgenic |
| | | | | | insert |
| TI-572 | TCCA | LbCas12a-TYC | 5233:5255 | − | Transgenic |
| | | | | | insert |
| TI-573 | TTTC | LbCas12a | 5240:5262 | − | Transgenic |
| | | | | | insert |
| TI-574 | TTCC | LbCas12a-TYC | 5254:5276 | − | Transgenic |
| | | | | | insert |
| TI-575 | TTTG | LbCas12a | 5263:5285 | − | Transgenic |
| | | | | | insert |
| TI-576 | TTTG | LbCas12a | 5267:5289 | − | Transgenic |
| | | | | | insert |
| TI-577 | TTCC | LbCas12a-TYC | 5271:5293 | − | Transgenic |
| | | | | | insert |
| TI-578 | TTTC | LbCas12a | 5272:5294 | − | Transgenic |
| | | | | | insert |
| TI-579 | TTTG | LbCas12a | 5307:5329 | + | Transgenic |
| | | | | | insert |
| TI-580 | TCCA | LbCas12a-TYC | 5314:5336 | + | Transgenic |
| | | | | | insert |
| TI-581 | TTCA | LbCas12a-TYC | 5298:5320 | − | Transgenic |
| | | | | | insert |
| TI-582 | CTTC | FnCas12a | 5299:5321 | − | Transgenic |
| | | | | | insert |
| TI-583 | TATC | LbCas12a-TATV | 5302:5324 | − | Transgenic |
| | | | | | insert |
| TI-584 | TATC | LbCas12a-TATV | 5308:5330 | − | Transgenic |
| | | | | | insert |
| TI-585 | TCCA | LbCas12a-TYC | 5316:5338 | − | Transgenic |
| | | | | | insert |
| TI-586 | TATC | LbCas12a-TATV | 5318:5340 | − | Transgenic |
| | | | | | insert |
| TI-587 | CTTA | FnCas12a | 5320:5342 | − | Transgenic |
| | | | | | insert |
| TI-588 | GTTC | FnCas12a | 5356:5378 | + | Transgenic |
| | | | | | insert |
| TI-589 | TTTG | LbCas12a | 5362:5384 | + | Transgenic |
| | | | | | insert |
| TI-590 | TTTA | LbCas12a | 5348:5370 | − | Transgenic |
| | | | | | insert |
| TI-591 | TTTA | LbCas12a | 5373:5395 | + | Transgenic |
| | | | | | insert |
| TI-592 | TTTG | LbCas12a | 5357:5379 | − | Transgenic |
| | | | | | insert |
| TI-593 | TATC | LbCas12a-TATV | 5396:5418 | + | Transgenic |
| | | | | | insert |
| TI-594 | CTTC | FnCas12a | 5399:5421 | + | Transgenic |
| | | | | | insert |
| TI-595 | TTCC | LbCas12a-TYC | 5400:5422 | + | Transgenic |
| | | | | | insert |
| TI-596 | TTTG | LbCas12a | 5383:5405 | − | Transgenic |
| | | | | | insert |
| TI-597 | TTCC | LbCas12a-TYC | 5419:5441 | + | Transgenic |
| | | | | | insert |
| TI-598 | TCCA | LbCas12a-TYC | 5420:5442 | + | Transgenic |
| | | | | | insert |
| TI-599 | GTTG | FnCas12a | 5426:5448 | + | Transgenic |
| | | | | | insert |
| TI-600 | CTTG | FnCas12a | 5407:5429 | − | Transgenic |
| | | | | | insert |
| TI-601 | CTTA | FnCas12a | 5439:5461 | + | Transgenic |
| | | | | | insert |
| TI-602 | TTCC | LbCas12a-TYC | 5443:5465 | + | Transgenic |
| | | | | | insert |
| TI-603 | TCCA | LbCas12a-TYC | 5444:5466 | + | Transgenic |
| | | | | | insert |
| TI-604 | GTTG | FnCas12a | 5428:5450 | − | Transgenic |
| | | | | | insert |
| TI-605 | TCCA | LbCas12a-TYC | 5453:5475 | + | Transgenic |
| | | | | | insert |
| TI-606 | TCCA | LbCas12a-TYC | 5456:5478 | − | Transgenic |
| | | | | | insert |
| TI-607 | TATC | LbCas12a-TATV | 5470:5492 | − | Transgenic |
| | | | | | insert |
| TI-608 | CTTG | FnCas12a | 5477:5499 | − | Transgenic |
| | | | | | insert |
| TI-609 | TATC | LbCas12a-TATV | 5517:5539 | − | Transgenic |
| | | | | | insert |
| TI-610 | TCCC | LbCas12a-TYC | 5529:5551 | − | Transgenic |
| | | | | | insert |
| TI-611 | TTTC | LbCas12a | 5533:5555 | − | Transgenic |
| | | | | | insert |
| TI-612 | GTTC | FnCas12a | 5568:5590 | + | Transgenic |
| | | | | | insert |
| TI-613 | CTTG | FnCas12a | 5551:5573 | − | Transgenic |
| | | | | | insert |
| TI-614 | TCCG | LbCas12a-TYC | 5574:5596 | − | Transgenic |
| | | | | | insert |
| TI-615 | TCCC | LbCas12a-TYC | 5610:5632 | + | Transgenic |
| | | | | | insert |
| TI-616 | GTTG | FnCas12a | 5618:5640 | + | Transgenic |
| | | | | | insert |
| TI-617 | CTTG | FnCas12a | 5629:5651 | − | Transgenic |
| | | | | | insert |
| TI-618 | CTTC | FnCas12a | 5686:5708 | + | Transgenic |
| | | | | | insert |
| TI-619 | CTTC | FnCas12a | 5689:5711 | + | Transgenic |
| | | | | | insert |
| TI-620 | TTCC | LbCas12a-TYC | 5690:5712 | + | Transgenic |
| | | | | | insert |
| TI-621 | GTTG | FnCas12a | 5674:5696 | − | Transgenic |
| | | | | | insert |
| TI-622 | CTTA | FnCas12a | 5696:5718 | + | Transgenic |
| | | | | | insert |
| TI-623 | TCCA | LbCas12a-TYC | 5680:5702 | − | Transgenic |
| | | | | | insert |
| TI-624 | CTTC | FnCas12a | 5687:5709 | − | Transgenic |
| | | | | | insert |
| TI-625 | TCCA | LbCas12a-TYC | 5700:5722 | − | Transgenic |
| | | | | | insert |
| TI-626 | TTCA | LbCas12a-TYC | 5712:5734 | − | Transgenic |
| | | | | | insert |
| TI-627 | GTTC | FnCas12a | 5713:5735 | − | Transgenic |
| | | | | | insert |
| TI-628 | CTTG | FnCas12a | 5743:5765 | + | Transgenic |
| | | | | | insert |
| TI-629 | GTTC | FnCas12a | 5755:5777 | + | Transgenic |
| | | | | | insert |
| TI-630 | GTTG | FnCas12a | 5734:5756 | − | Transgenic |
| | | | | | insert |
| TI-631 | CTTC | FnCas12a | 5758:5780 | + | Transgenic |
| | | | | | insert |
| TI-632 | TTCA | LbCas12a-TYC | 5759:5781 | + | Transgenic |
| | | | | | insert |
| TI-633 | TTCG | LbCas12a-TYC | 5746:5768 | − | Transgenic |
| | | | | | insert |
| TI-634 | GTTC | FnCas12a | 5747:5769 | − | Transgenic |
| | | | | | insert |
| TI-635 | TCCG | LbCas12a-TYC | 5750:5772 | − | Transgenic |
| | | | | | insert |
| TI-636 | TTCC | LbCas12a-TYC | 5751:5773 | − | Transgenic |
| | | | | | insert |
| TI-637 | CTTC | FnCas12a | 5752:5774 | − | Transgenic |
| | | | | | insert |
| TI-638 | TTTC | LbCas12a | 5758:5780 | − | Transgenic |
| | | | | | insert |
| TI-639 | GTTG | FnCas12a | 5767:5789 | − | Transgenic |
| | | | | | insert |
| TI-640 | TTCA | LbCas12a-TYC | 5789:5811 | + | Transgenic |
| | | | | | insert |
| TI-641 | CTTG | FnCas12a | 5797:5819 | + | Transgenic |
| | | | | | insert |
| TI-642 | CTTG | FnCas12a | 5782:5804 | − | Transgenic |
| | | | | | insert |
| TI-643 | GTTC | FnCas12a | 5785:5807 | − | Transgenic |
| | | | | | insert |
| TI-644 | CTTC | FnCas12a | 5815:5837 | + | Transgenic |
| | | | | | insert |
| TI-645 | TTCC | LbCas12a-TYC | 5816:5838 | + | Transgenic |
| | | | | | insert |
| TI-646 | TCCA | LbCas12a-TYC | 5817:5839 | + | Transgenic |
| | | | | | insert |
| TI-647 | CTTG | FnCas12a | 5798:5820 | − | Transgenic |
| | | | | | insert |
| TI-648 | TTCA | LbCas12a-TYC | 5823:5845 | + | Transgenic |
| | | | | | insert |
| TI-649 | TCCA | LbCas12a-TYC | 5806:5828 | − | Transgenic |
| | | | | | insert |
| TI-650 | TTCC | LbCas12a-TYC | 5807:5829 | − | Transgenic |
| | | | | | insert |
| TI-651 | GTTC | FnCas12a | 5808:5830 | − | Transgenic |
| | | | | | insert |
| TI-652 | TATG | LbCas12a-TATV | 5831:5853 | + | Transgenic |
| | | | | | insert |
| TI-653 | GTTA | FnCas12a | 5849:5871 | + | Transgenic |
| | | | | | insert |
| TI-654 | CTTG | FnCas12a | 5828:5850 | − | Transgenic |
| | | | | | insert |
| TI-655 | TCCA | LbCas12a-TYC | 5841:5863 | − | Transgenic |
| | | | | | insert |
| TI-656 | GTTG | FnCas12a | 5884:5906 | + | Transgenic |
| | | | | | insert |
| TI-657 | TTCA | LbCas12a-TYC | 5872:5894 | − | Transgenic |
| | | | | | insert |
| TI-658 | GTTC | FnCas12a | 5873:5895 | − | Transgenic |
| | | | | | insert |
| TI-659 | GTTG | FnCas12a | 5897:5919 | + | Transgenic |
| | | | | | insert |
| TI-660 | CTTG | FnCas12a | 5878:5900 | − | Transgenic |
| | | | | | insert |
| TI-661 | CTTG | FnCas12a | 5888:5910 | − | Transgenic |
| | | | | | insert |
| TI-662 | CTTG | FnCas12a | 5921:5943 | + | Transgenic |
| | | | | | insert |
| TI-663 | CTTC | FnCas12a | 5926:5948 | + | Transgenic |
| | | | | | insert |
| TI-664 | TTCA | LbCas12a-TYC | 5917:5939 | − | Transgenic |
| | | | | | insert |
| TI-665 | TTTC | LbCas12a | 5950:5972 | + | Transgenic |
| | | | | | insert |
| TI-666 | TCCC | LbCas12a-TYC | 5928:5950 | − | Transgenic |
| | | | | | insert |
| TI-667 | TTCA | LbCas12a-TYC | 5951:5973 | + | Transgenic |
| | | | | | insert |
| TI-668 | TTCA | LbCas12a-TYC | 5937:5959 | − | Transgenic |
| | | | | | insert |
| TI-669 | GTTC | FnCas12a | 5960:5982 | + | Transgenic |
| | | | | | insert |
| TI-670 | GTTC | FnCas12a | 5938:5960 | − | Transgenic |
| | | | | | insert |
| TI-671 | CTTG | FnCas12a | 5941:5963 | − | Transgenic |
| | | | | | insert |
| TI-672 | TATG | LbCas12a-TATV | 5972:5994 | + | Transgenic |
| | | | | | insert |
| TI-673 | TTCC | LbCas12a-TYC | 5954:5976 | − | Transgenic |
| | | | | | insert |
| TI-674 | GTTC | FnCas12a | 5955:5977 | − | Transgenic |
| | | | | | insert |
| TI-675 | TTTA | LbCas12a | 5959:5981 | − | Transgenic |
| | | | | | insert |
| TI-676 | TATC | LbCas12a-TATV | 5975:5997 | − | Transgenic |
| | | | | | insert |
| TI-677 | TATC | LbCas12a-TATV | 6004:6026 | + | Transgenic |
| | | | | | insert |
| TI-678 | TTCA | LbCas12a-TYC | 6009:6031 | + | Transgenic |
| | | | | | insert |
| TI-679 | TATG | LbCas12a-TATV | 6016:6038 | + | Transgenic |
| | | | | | insert |
| TI-680 | CTTG | FnCas12a | 5995:6017 | − | Transgenic |
| | | | | | insert |
| TI-681 | CTTC | FnCas12a | 6026:6048 | + | Transgenic |
| | | | | | insert |
| TI-682 | CTTG | FnCas12a | 6029:6051 | + | Transgenic |
| | | | | | insert |
| TI-683 | CTTG | FnCas12a | 6035:6057 | + | Transgenic |
| | | | | | insert |
| TI-684 | CTTC | FnCas12a | 6049:6071 | + | Transgenic |
| | | | | | insert |
| TI-685 | TCCA | LbCas12a-TYC | 6027:6049 | − | Transgenic |
| | | | | | insert |
| TI-686 | CTTG | FnCas12a | 6052:6074 | + | Transgenic |
| | | | | | insert |
| TI-687 | GTTG | FnCas12a | 6037:6059 | − | Transgenic |
| | | | | | insert |
| TI-688 | TTCG | LbCas12a-TYC | 6040:6062 | − | Transgenic |
| | | | | | insert |
| TI-689 | CTTC | FnCas12a | 6041:6063 | − | Transgenic |
| | | | | | insert |
| TI-690 | CTTG | FnCas12a | 6057:6079 | − | Transgenic |
| | | | | | insert |
| TI-691 | TTCG | LbCas12a-TYC | 6080:6102 | + | Transgenic |
| | | | | | insert |
| TI-692 | GTTG | FnCas12a | 6064:6086 | − | Transgenic |
| | | | | | insert |
| TI-693 | TTCA | LbCas12a-TYC | 6089:6111 | + | Transgenic |
| | | | | | insert |
| TI-694 | TCCC | LbCas12a-TYC | 6103:6125 | + | Transgenic |
| | | | | | insert |
| TI-695 | CTTG | FnCas12a | 6084:6106 | − | Transgenic |
| | | | | | insert |
| TI-696 | TCCA | LbCas12a-TYC | 6109:6131 | + | Transgenic |
| | | | | | insert |
| TI-697 | CTTG | FnCas12a | 6122:6144 | + | Transgenic |
| | | | | | insert |
| TI-698 | GTTC | FnCas12a | 6127:6149 | + | Transgenic |
| | | | | | insert |
| TI-699 | TTCG | LbCas12a-TYC | 6128:6150 | + | Transgenic |
| | | | | | insert |
| TI-700 | TCCG | LbCas12a-TYC | 6146:6168 | + | Transgenic |
| | | | | | insert |
| TI-701 | CTTG | FnCas12a | 6160:6182 | + | Transgenic |
| | | | | | insert |
| TI-702 | CTTC | FnCas12a | 6170:6192 | + | Transgenic |
| | | | | | insert |
| TI-703 | TTTG | LbCas12a | 6154:6176 | − | Transgenic |
| | | | | | insert |
| TI-704 | CTTG | FnCas12a | 6173:6195 | − | Transgenic |
| | | | | | insert |
| TI-705 | GTTC | FnCas12a | 6203:6225 | + | Transgenic |
| | | | | | insert |
| TI-706 | TTCA | LbCas12a-TYC | 6204:6226 | + | Transgenic |
| | | | | | insert |
| TI-707 | TCCC | LbCas12a-TYC | 6183:6205 | − | Transgenic |
| | | | | | insert |
| TI-708 | CTTG | FnCas12a | 6190:6212 | − | Transgenic |
| | | | | | insert |
| TI-709 | GTTG | FnCas12a | 6199:6221 | − | Transgenic |
| | | | | | insert |
| TI-710 | TCCA | LbCas12a-TYC | 6224:6246 | + | Transgenic |
| | | | | | insert |
| TI-711 | GTTG | FnCas12a | 6230:6252 | + | Transgenic |
| | | | | | insert |
| TI-712 | GTTG | FnCas12a | 6210:6232 | − | Transgenic |
| | | | | | insert |
| TI-713 | GTTG | FnCas12a | 6232:6254 | − | Transgenic |
| | | | | | insert |
| TI-714 | CTTA | FnCas12a | 6262:6284 | + | Transgenic |
| | | | | | insert |
| TI-715 | TCCA | LbCas12a-TYC | 6249:6271 | − | Transgenic |
| | | | | | insert |
| TI-716 | CTTC | FnCas12a | 6298:6320 | + | Transgenic |
| | | | | | insert |
| TI-717 | TTCA | LbCas12a-TYC | 6299:6321 | + | Transgenic |
| | | | | | insert |
| TI-718 | TTTG | LbCas12a | 6300:6322 | − | Transgenic |
| | | | | | insert |
| TI-719 | CTTC | FnCas12a | 6310:6332 | − | Transgenic |
| | | | | | insert |
| TI-720 | TCCC | LbCas12a-TYC | 6350:6372 | + | Transgenic |
| | | | | | insert |
| TI-721 | TATA | LbCas12a-TATV | 6336:6358 | − | Transgenic |
| | | | | | insert |
| TI-722 | GTTA | FnCas12a | 6361:6383 | + | Transgenic |
| | | | | | insert |
| TI-723 | TATA | LbCas12a-TATV | 6363:6385 | + | Transgenic |
| | | | | | insert |
| TI-724 | TTCA | LbCas12a-TYC | 6345:6367 | − | Transgenic |
| | | | | | insert |
| TI-725 | GTTC | FnCas12a | 6346:6368 | − | Transgenic |
| | | | | | insert |
| TI-726 | TTTC | LbCas12a | 6355:6377 | − | Transgenic |
| | | | | | insert |
| TI-727 | TTTG | LbCas12a | 6406:6428 | + | Transgenic |
| | | | | | insert |
| TI-728 | TCCA | LbCas12a-TYC | 6384:6406 | − | Transgenic |
| | | | | | insert |
| TI-729 | CTTG | FnCas12a | 6402:6424 | − | Transgenic |
| | | | | | insert |
| TI-730 | TATC | LbCas12a-TATV | 6405:6427 | − | Transgenic |
| | | | | | insert |
| TI-731 | TTCC | LbCas12a-TYC | 6411:6433 | − | Transgenic |
| | | | | | insert |
| TI-732 | TTTC | LbCas12a | 6412:6434 | − | Transgenic |
| | | | | | insert |
| TI-733 | TTCG | LbCas12a-TYC | 6424:6446 | − | Transgenic |
| | | | | | insert |
| TI-734 | CTTC | FnCas12a | 6425:6447 | − | Transgenic |
| | | | | | insert |
| TI-735 | GTTC | FnCas12a | 6457:6479 | + | Transgenic |
| | | | | | insert |
| TI-736 | TTCA | LbCas12a-TYC | 6458:6480 | + | Transgenic |
| | | | | | insert |
| TI-737 | TCCC | LbCas12a-TYC | 6440:6462 | − | Transgenic |
| | | | | | insert |
| TI-738 | TCCA | LbCas12a-TYC | 6448:6470 | − | Transgenic |
| | | | | | insert |
| TI-739 | TTCC | LbCas12a-TYC | 6449:6471 | − | Transgenic |
| | | | | | insert |
| TI-740 | TTTC | LbCas12a | 6450:6472 | − | Transgenic |
| | | | | | insert |
| TI-741 | TTTG | LbCas12a | 6473:6495 | + | Transgenic |
| | | | | | insert |
| TI-742 | TCCC | LbCas12a-TYC | 6509:6531 | + | Transgenic |
| | | | | | insert |
| TI-743 | CTTA | FnCas12a | 6503:6525 | − | Transgenic |
| | | | | | insert |
| TI-744 | TATG | LbCas12a-TATV | 6525:6547 | − | Transgenic |
| | | | | | insert |
| TI-745 | TTTC | LbCas12a | 6549:6571 | + | Transgenic |
| | | | | | insert |
| TI-746 | TTCA | LbCas12a-TYC | 6550:6572 | + | Transgenic |
| | | | | | insert |
| TI-747 | TTTC | LbCas12a | 6557:6579 | + | Transgenic |
| | | | | | insert |
| TI-748 | TTCA | LbCas12a-TYC | 6558:6580 | + | Transgenic |
| | | | | | insert |
| TI-749 | TATG | LbCas12a-TATV | 6585:6607 | + | Transgenic |
| | | | | | insert |
| TI-750 | GTTC | FnCas12a | 6569:6591 | − | Transgenic |
| | | | | | insert |
| TI-751 | CTTA | FnCas12a | 6604:6626 | + | Transgenic |
| | | | | | insert |
| TI-752 | TTTA | LbCas12a | 6619:6641 | + | Transgenic |
| | | | | | insert |
| TI-753 | GTTA | FnCas12a | 6632:6654 | + | Transgenic |
| | | | | | insert |
| TI-754 | TTCA | LbCas12a-TYC | 6613:6635 | − | Transgenic |
| | | | | | insert |
| TI-755 | GTTC | FnCas12a | 6614:6636 | − | Transgenic |
| | | | | | insert |
| TI-756 | TTTG | LbCas12a | 6618:6640 | − | Transgenic |
| | | | | | insert |
| TI-757 | CTTA | FnCas12a | 6650:6672 | + | Transgenic |
| | | | | | insert |
| TI-758 | TATC | LbCas12a-TATV | 6652:6674 | + | Transgenic |
| | | | | | insert |
| TI-759 | TTTA | LbCas12a | 6638:6660 | − | Transgenic |
| | | | | | insert |
| TI-760 | CTTA | FnCas12a | 6647:6669 | − | Transgenic |
| | | | | | insert |
| TI-761 | CTTG | FnCas12a | 6680:6702 | + | Transgenic |
| | | | | | insert |
| TI-762 | TTTG | LbCas12a | 6684:6706 | + | Transgenic |
| | | | | | insert |
| TI-763 | CTTC | FnCas12a | 6691:6713 | + | Transgenic |
| | | | | | insert |
| TI-764 | TTTG | LbCas12a | 6695:6717 | + | Transgenic |
| | | | | | insert |
| TI-765 | TCCC | LbCas12a-TYC | 6703:6725 | + | Transgenic |
| | | | | | insert |
| TI-766 | TTTG | LbCas12a | 6707:6729 | + | Transgenic |
| | | | | | insert |
| TI-767 | CTTA | FnCas12a | 6713:6735 | + | Transgenic |
| | | | | | insert |
| TI-768 | TTTA | LbCas12a | 6726:6748 | + | Transgenic |
| | | | | | insert |
| TI-769 | GTTG | FnCas12a | 6732:6754 | + | Transgenic |
| | | | | | insert |
| TI-770 | TTTG | LbCas12a | 6740:6762 | + | Transgenic |
| | | | | | insert |
| TI-771 | TATG | LbCas12a-TATV | 6718:6740 | − | Transgenic |
| | | | | | insert |
| TI-772 | CTTC | FnCas12a | 6751:6773 | + | Transgenic |
| | | | | | insert |
| TI-773 | CTTG | FnCas12a | 6756:6778 | + | Transgenic |
| | | | | | insert |
| TI-774 | TTTG | LbCas12a | 6752:6774 | − | Transgenic |
| | | | | | insert |
| TI-775 | TTTC | LbCas12a | 6784:6806 | + | Transgenic |
| | | | | | insert |
| TI-776 | TTCA | LbCas12a-TYC | 6785:6807 | + | Transgenic |
| | | | | | insert |
| TI-777 | TTTG | LbCas12a | 6767:6789 | − | Transgenic |
| | | | | | insert |
| TI-778 | TATC | LbCas12a-TATV | 6791:6813 | + | Transgenic |
| | | | | | insert |
| TI-779 | TATG | LbCas12a-TATV | 6804:6826 | + | Transgenic |
| | | | | | insert |
| TI-780 | GTTC | FnCas12a | 6807:6829 | + | Transgenic |
| | | | | | insert |
| TI-781 | CTTA | FnCas12a | 6815:6837 | + | Transgenic |
| | | | | | insert |
| TI-782 | GTTA | FnCas12a | 6795:6817 | − | Transgenic |
| | | | | | insert |
| TI-783 | TATG | LbCas12a-TATV | 6817:6839 | + | Transgenic |
| | | | | | insert |
| TI-784 | CTTA | FnCas12a | 6828:6850 | + | Transgenic |
| | | | | | insert |
| TI-785 | TCCA | LbCas12a-TYC | 6810:6832 | − | Transgenic |
| | | | | | insert |
| TI-786 | TTCC | LbCas12a-TYC | 6811:6833 | − | Transgenic |
| | | | | | insert |
| TI-787 | CTTC | FnCas12a | 6812:6834 | − | Transgenic |
| | | | | | insert |
| TI-788 | TTTG | LbCas12a | 6874:6896 | + | Transgenic |
| | | | | | insert |
| TI-789 | TTCA | LbCas12a-TYC | 6870:6892 | − | Transgenic |
| | | | | | insert |
| TI-790 | TTTG | LbCas12a-TYC | 6880:6902 | − | Transgenic |
| | | | | | insert |
| TI-791 | TTTG | LbCas12a | 6929:6951 | + | Transgenic |
| | | | | | insert |
| TI-792 | TTCA | LbCas12a-TYC | 6907:6929 | − | Transgenic |
| | | | | | insert |
| TI-793 | CTTC | FnCas12a | 6908:6930 | − | Transgenic |
| | | | | | insert |
| TI-794 | CTTA | FnCas12a | 6916:6938 | − | Transgenic |
| | | | | | insert |
| TI-795 | TTTA | LbCas12a | 6954:6976 | + | Transgenic |
| | | | | | insert |
| TI-796 | TTTC | LbCas12a | 6963:6985 | + | Transgenic |
| | | | | | insert |
| TI-797 | TTCA | LbCas12a-TYC | 6964:6986 | + | Transgenic |
| | | | | | insert |
| TI-798 | GTTG | FnCas12a | 6975:6997 | + | Transgenic |
| | | | | | insert |
| TI-799 | TTTG | LbCas12a | 6961:6983 | − | Transgenic |
| | | | | | insert |
| TI-800 | TATG | LbCas12a-TATV | 6983:7005 | + | Transgenic |
| | | | | | insert |
| TI-801 | TTCG | LbCas12a-TYC | 6972:6994 | − | Transgenic |
| | | | | | insert |
| TI-802 | GTTC | FnCas12a | 7007:7029 | + | Transgenic |
| | | | | | insert |
| TI-803 | CTTA | FnCas12a | 7010:7032 | + | Transgenic |
| | | | | | insert |
| TI-804 | TTTC | LbCas12a | 7019:7041 | + | Transgenic |
| | | | | | insert |
| TI-805 | TTCA | LbCas12a-TYC | 7020:7042 | + | Transgenic |
| | | | | | insert |
| TI-806 | TTTG | LbCas12a | 7041:7063 | + | Transgenic |
| | | | | | insert |
| TI-807 | TTCA | LbCas12a-TYC | 7046:7068 | − | Transgenic |
| | | | | | insert |
| TI-808 | TTTC | LbCas12a | 7047:7069 | − | Transgenic |
| | | | | | insert |
| TI-809 | GTTC | FnCas12a | 7073:7095 | − | Transgenic |
| | | | | | insert |
| TI-810 | TTTA | LbCas12a | 7085:7107 | − | Transgenic |
| | | | | | insert |
| TI-811 | GTTA | FnCas12a | 7110:7132 | + | Transgenic |
| | | | | | insert |
| TI-812 | TCCA | LbCas12a-TYC | 7091:7113 | − | Transgenic |
| | | | | | insert |
| TI-813 | TATG | LbCas12a-TATV | 7116:7138 | + | Transgenic |
| | | | | | insert |
| TI-814 | TTTG | LbCas12a | 7103:7125 | − | Transgenic |
| | | | | | insert |
| TI-815 | CTTA | FnCas12a | 7107:7129 | − | Transgenic |
| | | | | | insert |
| TI-816 | TATC | LbCas12a-TATV | 7118:7140 | − | Transgenic |
| | | | | | insert |
| TI-817 | TATA | LbCas12a-TATV | 7120:7142 | − | Transgenic |
| | | | | | insert |
| TI-818 | TTCA | LbCas12a-TYC | 7124:7146 | − | Transgenic |
| | | | | | insert |
| TI-819 | TTTA | LbCas12a | 7129:7151 | − | Transgenic |
| | | | | | insert |
| TI-820 | TTTG | LbCas12a | 7155:7177 | − | Transgenic |
| | | | | | insert |
| TI-821 | TTTA | LbCas12a | 7198:7220 | + | Transgenic |
| | | | | | insert |
| TI-822 | CTTG | FnCas12a | 7208:7230 | + | Transgenic |
| | | | | | insert |
| TI-823 | TTCA | LbCas12a-TYC | 7212:7234 | + | Transgenic |
| | | | | | insert |
| TI-824 | CTTG | FnCas12a | 7220:7242 | + | Transgenic |
| | | | | | insert |
| TI-825 | TTTG | LbCas12a | 7200:7222 | − | Transgenic |
| | | | | | insert |
| TI-826 | TATC | LbCas12a-TATV | 7237:7259 | + | Transgenic |
| | | | | | insert |
| TI-827 | TTTG | LbCas12a | 7219:7241 | − | Transgenic |
| | | | | | insert |
| TI-828 | TATG | LbCas12a-TATV | 7231:7253 | − | Transgenic |
| | | | | | insert |
| TI-829 | TTTG | LbCas12a | 7235:7257 | − | Transgenic |
| | | | | | insert |
| TI-830 | TTTC | LbCas12a | 7305:7327 | + | Transgenic |
| | | | | | insert |
| TI-831 | TTCA | LbCas12a-TYC | 7306:7328 | + | Transgenic |
| | | | | | insert |
| TI-832 | TATA | LbCas12a-TATV | 7291:7313 | − | Transgenic |
| | | | | | insert |
| TI-833 | TATG | LbCas12a-TATV | 7313:7335 | + | Transgenic |
| | | | | | insert |
| TI-834 | GTTA | FnCas12a | 7316:7338 | + | Transgenic |
| | | | | | insert |
| TI-835 | TATA | LbCas12a-TATV | 7318:7340 | + | Transgenic |
| | | | | | insert |
| TI-836 | TATC | LbCas12a-TATV | 7296:7318 | − | Transgenic |
| | | | | | insert |
| TI-837 | TTTA | LbCas12a | 7298:7320 | − | Transgenic |
| | | | | | insert |
| TI-838 | CTTC | FnCas12a | 7302:7324 | − | Transgenic |
| | | | | | insert |
| TI-839 | GTTA | FnCas12a | 7332:7354 | + | Transgenic |
| | | | | | insert |
| TI-840 | CTTG | FnCas12a | 7315:7337 | − | Transgenic |
| | | | | | insert |
| TI-841 | TTTC | LbCas12a | 7338:7360 | + | Transgenic |
| | | | | | insert |
| TI-842 | TTCC | LbCas12a-TYC | 7339:7361 | + | Transgenic |
| | | | | | insert |
| TI-843 | TTTC | LbCas12a | 7318:7340 | − | Transgenic |
| | | | | | insert |
| TI-844 | TCCA | LbCas12a-TYC | 7340:7362 | + | Transgenic |
| | | | | | insert |
| TI-845 | TTCA | LbCas12a-TYC | 7349:7371 | + | Transgenic |
| | | | | | insert |
| TI-846 | TATA | LbCas12a-TATV | 7333:7355 | − | Transgenic |
| | | | | | insert |
| TI-847 | CTTA | FnCas12a | 7335:7357 | − | Transgenic |
| | | | | | insert |
| TI-848 | TTTA | LbCas12a | 7358:7380 | + | Transgenic |
| | | | | | insert |
| TI-849 | TATA | LbCas12a-TATV | 7360:7382 | + | Transgenic |
| | | | | | insert |
| TI-850 | TATC | LbCas12a-TATV | 7352:7374 | − | Transgenic |
| | | | | | insert |
| TI-851 | TTTA | LbCas12a | 7354:7376 | − | Transgenic |
| | | | | | insert |
| TI-852 | TTTG | LbCas12a | 7361:7383 | − | Transgenic |
| | | | | | insert |
| TI-853 | TTTC | LbCas12a | 7406:7428 | − | Transgenic |
| | | | | | insert |
| TI-854 | TTCG | LbCas12a-TYC | 7413:7435 | − | Transgenic |
| | | | | | insert |
| TI-855 | TTTC | LbCas12a | 7414:7436 | − | Transgenic |
| | | | | | insert |
| TI-856 | CTTC | FnCas12a | 7449:7471 | + | Transgenic |
| | | | | | insert |
| TI-857 | TTCA | LbCas12a-TYC | 7450:7472 | + | Transgenic |
| | | | | | insert |
| TI-858 | TCCC | LbCas12a-TYC | 7434:7456 | − | Transgenic |
| | | | | | insert |
| TI-859 | GTTC | FnCas12a | 7471:7493 | + | Transgenic |
| | | | | | insert |
| TI-860 | TTCA | LbCas12a-TYC | 7472:7494 | + | Transgenic |
| | | | | | insert |
| TI-861 | TATA | LbCas12a-TATV | 7475:7497 | − | Transgenic |
| | | | | | insert |
| TI-862 | TTTA | LbCas12a | 7481:7503 | − | Transgenic |
| | | | | | insert |
| TI-863 | TATA | LbCas12a-TATV | 7537:7559 | + | Transgenic |
| | | | | | insert |
| TI-864 | TTTA | LbCas12a | 7529:7551 | − | Transgenic |
| | | | | | insert |
| TI-865 | GTTA | FnCas12a | 7554:7576 | + | Transgenic |
| | | | | | insert |
| TI-866 | GTTG | FnCas12a | 7536:7558 | − | Transgenic |
| | | | | | insert |
| TI-867 | TCCA | LbCas12a-TYC | 7543:7565 | − | Transgenic |
| | | | | | insert |
| TI-868 | CTTA | FnCas12a | 7575:7597 | + | Transgenic |
| | | | | | insert |
| TI-869 | TATG | LbCas12a-TATV | 7577:7599 | + | Transgenic |
| | | | | | insert |
| TI-870 | TTTC | LbCas12a | 7564:7586 | − | Transgenic |
| | | | | | insert |
| TI-871 | GTTA | FnCas12a | 7587:7609 | + | Transgenic |
| | | | | | insert |
| TI-872 | GTTG | FnCas12a | 7600:7622 | + | Transgenic |
| | | | | | insert |
| TI-873 | TTTA | LbCas12a-TYC | 7592:7614 | − | Transgenic |
| | | | | | insert |
| TI-874 | TTTG | LbCas12a | 7602:7624 | − | Transgenic |
| | | | | | insert |
| TI-875 | GTTG | FnCas12a | 7636:7658 | + | Transgenic |
| | | | | | insert |
| TI-876 | TTTA | LbCas12a | 7662:7684 | + | Transgenic |
| | | | | | insert |
| TI-877 | GTTG | FnCas12a | 7640:7662 | − | Transgenic |
| | | | | | insert |
| TI-878 | TATC | LbCas12a-TATV | 7664:7686 | + | Transgenic |
| | | | | | insert |
| TI-879 | TTTG | LbCas12a | 7645:7667 | − | Transgenic |
| | | | | | insert |
| TI-880 | TTTG | LbCas12a | 7652:7674 | − | Transgenic |
| | | | | | insert |
| TI-881 | TTTA | LbCas12a | 7671:7693 | − | Transgenic |
| | | | | | insert |
| TI-882 | TTTG | LbCas12a | 7727:7749 | + | Transgenic |
| | | | | | insert |
| TI-883 | TTTA | LbCas12a | 7761:7783 | + | Transgenic |
| | | | | | insert |
| TI-884 | CTTC | FnCas12a | 7778:7800 | + | Transgenic |
| | | | | | insert |
| TI-885 | TTCA | LbCas12a-TYC | 7779:7801 | + | Transgenic |
| | | | | | insert |
| TI-886 | CTTA | FnCas12a | 7787:7809 | + | Transgenic |
| | | | | | insert |
| TI-887 | TTTC | LbCas12a | 7793:7815 | + | Transgenic |
| | | | | | insert |
| TI-888 | CTTC | FnCas12a | 7823:7845 | + | Transgenic |
| | | | | | insert |
| TI-889 | CTTC | FnCas12a | 7826:7848 | + | Transgenic |
| | | | | | insert |
| TI-890 | CTTC | FnCas12a | 7829:7851 | + | Transgenic |
| | | | | | insert |
| TI-891 | CTTC | FnCas12a | 7832:7854 | + | Transgenic |
| | | | | | insert |
| TI-892 | TTTA | LbCas12a-TYC | 7811:7833 | − | Transgenic |
| | | | | | insert |
| TI-893 | TATA | LbCas12a-TATV | 7836:7858 | + | Transgenic |
| | | | | | insert |
| TI-894 | TTTG | LbCas12a | 7823:7845 | − | Transgenic |
| | | | | | insert |
| TI-895 | CTTC | FnCas12a | 7865:7887 | + | Transgenic |
| | | | | | insert |
| TI-896 | TTCA | LbCas12a-TYC | 7866:7888 | + | Transgenic |
| | | | | | insert |
| TI-897 | TTCA | LbCas12a-TYC | 7873:7895 | + | Transgenic |
| | | | | | insert |
| TI-898 | TTTG | LbCas12a | 7863:7885 | − | Transgenic |
| | | | | | insert |
| TI-899 | TTTA | LbCas12a-TYC | 7871:7893 | − | Transgenic |
| | | | | | insert |
| TI-900 | TTTC | LbCas12a | 7905:7927 | + | Transgenic |
| | | | | | insert |
| TI-901 | CTTG | FnCas12a | 7904:7926 | − | Transgenic |
| | | | | | insert |
| TI-902 | TTTA | LbCas12a | 7909:7931 | − | Transgenic |
| | | | | | insert |
| TI-903 | TTTC | LbCas12a | 7940:7962 | + | Transgenic |
| | | | | | insert |
| TI-904 | GTTC | FnCas12a | 7947:7969 | + | Transgenic |
| | | | | | insert |
| TI-905 | TTCC | LbCas12a-TYC | 7948:7970 | + | Transgenic |
| | | | | | insert |
| TI-906 | TTTG | LbCas12a-TYC | 7934:7956 | − | Transgenic |
| | | | | | insert |
| TI-907 | CTTC | FnCas12a | 7967:7989 | + | Transgenic |
| | | | | | insert |
| TI-908 | TTCG | LbCas12a-TYC | 7968:7990 | + | Transgenic |
| | | | | | insert |
| TI-909 | TTTG | LbCas12a | 7974:7996 | + | Transgenic |
| | | | | | insert |
| TI-910 | TTTC | LbCas12a | 7979:8001 | + | Transgenic |
| | | | | | insert |
| TI-911 | TTCG | LbCas12a-TYC | 7980:8002 | + | Transgenic |
| | | | | | insert |
| TI-912 | GTTC | FnCas12a | 7983:8005 | + | Transgenic |
| | | | | | insert |
| TI-913 | TTCG | LbCas12a-TYC | 7984:8006 | + | Transgenic |
| | | | | | insert |
| TI-914 | TTTC | LbCas12a | 7995:8017 | + | Transgenic |
| | | | | | insert |
| TI-915 | TATA | LbCas12a-TATV | 7973:7995 | − | Transgenic |
| | | | | | insert |
| TI-916 | TTCG | LbCas12a-TYC | 7996:8018 | + | Transgenic |
| | | | | | insert |
| TI-917 | TATA | LbCas12a-TATV | 8000:8022 | + | Transgenic |
| | | | | | insert |
| TI-918 | GTTC | FnCas12a | 8005:8027 | + | Transgenic |
| | | | | | insert |
| TI-919 | TTTG | LbCas12a | 8009:8031 | + | Transgenic |
| | | | | | insert |
| TI-920 | TTTA | LbCas12a | 8014:8036 | + | Transgenic |
| | | | | | insert |
| TI-921 | GTTA | FnCas12a | 8024:8046 | + | Transgenic |
| | | | | | insert |
| TI-922 | CTTA | FnCas12a | 8030:8052 | + | Transgenic |
| | | | | | insert |
| TI-923 | TTCG | LbCas12a-TYC | 8010:8032 | − | Transgenic |
| | | | | | insert |
| TI-924 | CTTC | FnCas12a | 8011:8033 | − | Transgenic |
| | | | | | insert |
| TI-925 | TTTC | LbCas12a | 8047:8069 | + | Transgenic |
| | | | | | insert |
| TI-926 | TTTG | LbCas12a | 8055:8077 | + | Transgenic |
| | | | | | insert |
| TI-927 | TATC | LbCas12a-TATV | 8039:8061 | − | Transgenic |
| | | | | | insert |
| TI-928 | GTTA | FnCas12a | 8062:8084 | + | Transgenic |
| | | | | | insert |
| TI-929 | TATC | LbCas12a-TATV | 8068:8090 | + | Transgenic |
| | | | | | insert |
| TI-930 | CTTA | FnCas12a | 8074:8096 | + | Transgenic |
| | | | | | insert |
| TI-931 | TATG | LbCas12a-TATV | 8068:8090 | − | Transgenic |
| | | | | | insert |
| TI-932 | TTTC | LbCas12a | 8092:8114 | + | Transgenic |
| | | | | | insert |
| TI-933 | TTCA | LbCas12a-TYC | 8093:8115 | + | Transgenic |
| | | | | | insert |
| TI-934 | TATC | LbCas12a-TATV | 8072:8094 | − | Transgenic |
| | | | | | insert |
| TI-935 | TATC | LbCas12a-TATV | 8101:8123 | + | Transgenic |
| | | | | | insert |
| TI-936 | TTTG | LbCas12a | 8111:8133 | + | Transgenic |
| | | | | | insert |
| TI-937 | TTTG | LbCas12a | 8090:8112 | − | Transgenic |
| | | | | | insert |
| TI-938 | GTTC | FnCas12a | 8114:8136 | + | Transgenic |
| | | | | | insert |
| TI-939 | TTTG | LbCas12a | 8124:8146 | + | Transgenic |
| | | | | | insert |
| TI-940 | TTTG | LbCas12a | 8131:8153 | + | Transgenic |
| | | | | | insert |
| TI-941 | TTCG | LbCas12a-TYC | 8109:8131 | − | Transgenic |
| | | | | | insert |
| TI-942 | CTTC | FnCas12a | 8148:8170 | + | Transgenic |
| | | | | | insert |
| TI-943 | TTCG | LbCas12a-TYC | 8149:8171 | + | Transgenic |
| | | | | | insert |
| TI-944 | TTTG | LbCas12a | 8154:8176 | + | Transgenic |
| | | | | | insert |
| TI-945 | TTTC | LbCas12a | 8161:8183 | + | Transgenic |
| | | | | | insert |
| TI-946 | TATC | LbCas12a-TATV | 8165:8187 | + | Transgenic |
| | | | | | insert |
| TI-947 | GTTA | FnCas12a | 8179:8201 | + | Transgenic |
| | | | | | insert |
| TI-948 | TTTC | LbCas12a | 8184:8206 | + | Transgenic |
| | | | | | insert |
| TI-949 | TTTG | LbCas12a | 8191:8213 | + | Transgenic |
| | | | | | insert |
| TI-950 | TTCG | LbCas12a-TYC | 8174:8196 | − | Transgenic |
| | | | | | insert |
| TI-951 | TTTC | LbCas12a | 8225:8247 | + | Transgenic |
| | | | | | insert |
| TI-952 | TATG | LbCas12a-TATV | 8240:8262 | + | Transgenic |
| | | | | | insert |
| TI-953 | TTTA | LbCas12a | 8274:8296 | + | Transgenic |
| | | | | | insert |
| TI-954 | TTCC | LbCas12a-TYC | 8268:8290 | − | Transgenic |
| | | | | | insert |
| TI-955 | GTTC | FnCas12a | 8269:8291 | − | Transgenic |
| | | | | | insert |
| TI-956 | TCCG | LbCas12a-TYC | 8272:8294 | − | Transgenic |
| | | | | | insert |
| TI-957 | TTCG | LbCas12a-TYC | 8280:8302 | − | Transgenic |
| | | | | | insert |
| TI-958 | CTTC | FnCas12a | 8281:8303 | − | Transgenic |
| | | | | | insert |
| TI-959 | TTTA | LbCas12a-TYC | 8314:8336 | + | Transgenic |
| | | | | | insert |
| TI-960 | TTCG | LbCas12a-TYC | 8318:8340 | + | Transgenic |
| | | | | | insert |
| TI-961 | TTTC | LbCas12a | 8323:8345 | + | Transgenic |
| | | | | | insert |
| TI-962 | GTTG | FnCas12a | 8307:8329 | − | Transgenic |
| | | | | | insert |
| TI-963 | CTTC | FnCas12a | 8331:8353 | + | Transgenic |
| | | | | | insert |
| TI-964 | TTCA | LbCas12a-TYC | 8332:8354 | + | Transgenic |
| | | | | | insert |
| TI-965 | CTTC | FnCas12a | 8338:8360 | + | Transgenic |
| | | | | | insert |
| TI-966 | TTTC | LbCas12a | 8349:8371 | + | Transgenic |
| | | | | | insert |
| TI-967 | TTCC | LbCas12a-TYC | 8350:8372 | + | Transgenic |
| | | | | | insert |
| TI-968 | TCCG | LbCas12a-TYC | 8351:8373 | + | Transgenic |
| | | | | | insert |
| TI-969 | TTCG | LbCas12a-TYC | 8329:8351 | − | Transgenic |
| | | | | | insert |
| TI-970 | CTTC | FnCas12a | 8330:8352 | − | Transgenic |
| | | | | | insert |
| TI-971 | TTCC | LbCas12a-TYC | 8366:8388 | + | Transgenic |
| | | | | | insert |
| TI-972 | TTTG | LbCas12a | 8345:8367 | − | Transgenic |
| | | | | | insert |
| TI-973 | CTTC | FnCas12a | 8369:8391 | + | Transgenic |
| | | | | | insert |
| TI-974 | TTCA | LbCas12a-TYC | 8370:8392 | + | Transgenic |
| | | | | | insert |
| TI-975 | TTTG | LbCas12a | 8354:8376 | − | Transgenic |
| | | | | | insert |
| TI-976 | TTTA | LbCas12a | 8359:8381 | − | Transgenic |
| | | | | | insert |
| TI-977 | CTTG | FnCas12a | 8415:8437 | + | Transgenic |
| | | | | | insert |
| TI-978 | GTTC | FnCas12a | 8429:8451 | + | Transgenic |
| | | | | | insert |
| TI-979 | TTCG | LbCas12a-TYC | 8430:8452 | + | Transgenic |
| | | | | | insert |
| TI-980 | GTTG | FnCas12a | 8433:8455 | + | Transgenic |
| | | | | | insert |
| TI-981 | TCCG | LbCas12a-TYC | 8437:8459 | + | Transgenic |
| | | | | | insert |
| TI-982 | TATG | LbCas12a-TATV | 8444:8466 | + | Transgenic |
| | | | | | insert |
| TI-983 | TTTC | LbCas12a | 8453:8475 | + | Transgenic |
| | | | | | insert |
| TI-984 | TTCG | LbCas12a-TYC | 8454:8476 | + | Transgenic |
| | | | | | insert |
| TI-985 | TTTC | LbCas12a | 8434:8456 | − | Transgenic |
| | | | | | insert |
| TI-986 | GTTA | FnCas12a | 8457:8479 | + | Transgenic |
| | | | | | insert |
| TI-987 | CTTG | FnCas12a | 8467:8489 | + | Transgenic |
| | | | | | insert |
| TI-988 | GTTG | FnCas12a | 8475:8497 | + | Transgenic |
| | | | | | insert |
| TI-989 | CTTC | FnCas12a | 8484:8506 | + | Transgenic |
| | | | | | insert |
| TI-990 | TTCC | LbCas12a-TYC | 8485:8507 | + | Transgenic |
| | | | | | insert |
| TI-991 | GTTG | FnCas12a | 8495:8517 | + | Transgenic |
| | | | | | insert |
| TI-992 | CTTC | FnCas12a | 8477:8499 | − | Transgenic |
| | | | | | insert |
| TI-993 | TTCC | LbCas12a-TYC | 8487:8509 | − | Transgenic |
| | | | | | insert |
| TI-994 | CTTC | FnCas12a | 8488:8510 | − | Transgenic |
| | | | | | insert |
| TI-995 | GTTC | FnCas12a | 8491:8513 | − | Transgenic |
| | | | | | insert |
| TI-996 | TATC | LbCas12a-TATV | 8522:8544 | + | Transgenic |
| | | | | | insert |
| TI-997 | TATC | LbCas12a-TATV | 8502:8524 | − | Transgenic |
| | | | | | insert |
| TI-998 | TCCA | LbCas12a-TYC | 8534:8556 | + | Transgenic |
| | | | | | insert |
| TI-999 | TATC | LbCas12a-TATV | 8547:8569 | + | Transgenic |
| | | | | | insert |
| TI-1000 | GTTG | FnCas12a | 8526:8548 | − | Transgenic |
| | | | | | insert |
| TI-1001 | TCCG | LbCas12a-TYC | 8534:8556 | − | Transgenic |
| | | | | | insert |
| TI-1002 | GTTG | FnCas12a | 8565:8587 | + | Transgenic |
| | | | | | insert |
| TI-1003 | TATC | LbCas12a-TATV | 8556:8578 | − | Transgenic |
| | | | | | insert |
| TI-1004 | CTTG | FnCas12a | 8580:8602 | + | Transgenic |
| | | | | | insert |
| TI-1005 | TTTG | LbCas12a | 8587:8609 | + | Transgenic |
| | | | | | insert |
| TI-1006 | TCCG | LbCas12a-TYC | 8591:8613 | + | Transgenic |
| | | | | | insert |
| TI-1007 | CTTC | FnCas12a | 8600:8622 | + | Transgenic |
| | | | | | insert |
| TI-1008 | TTCG | LbCas12a-TYC | 8601:8623 | + | Transgenic |
| | | | | | insert |
| TI-1009 | TCCG | LbCas12a-TYC | 8606:8628 | + | Transgenic |
| | | | | | insert |
| TI-1010 | GTTG | FnCas12a | 8609:8631 | + | Transgenic |
| | | | | | insert |
| TI-1011 | GTTG | FnCas12a | 8603:8625 | − | Transgenic |
| | | | | | insert |
| TI-1012 | TCCG | LbCas12a-TYC | 8606:8628 | − | Transgenic |
| | | | | | insert |
| TI-1013 | TCCA | LbCas12a-TYC | 8641:8663 | + | Transgenic |
| | | | | | insert |
| TI-1014 | TATG | LbCas12a-TATV | 8623:8645 | − | Transgenic |
| | | | | | insert |
| TI-1015 | TCCA | LbCas12a-TYC | 8648:8670 | + | Transgenic |
| | | | | | insert |
| TI-1016 | TATC | LbCas12a-TATV | 8652:8674 | + | Transgenic |
| | | | | | insert |
| TI-1017 | TCCA | LbCas12a-TYC | 8635:8657 | − | Transgenic |
| | | | | | insert |
| TI-1018 | TTCC | LbCas12a-TYC | 8636:8658 | − | Transgenic |
| | | | | | insert |
| TI-1019 | TTTG | LbCas12a | 8667:8689 | + | Transgenic |
| | | | | | insert |
| TI-1020 | TCCG | LbCas12a-TYC | 8645:8667 | − | Transgenic |
| | | | | | insert |
| TI-1021 | GTTG | FnCas12a | 8666:8688 | − | Transgenic |
| | | | | | insert |
| TI-1022 | TCCA | LbCas12a-TYC | 8689:8711 | + | Transgenic |
| | | | | | insert |
| TI-1023 | GTTG | FnCas12a | 8678:8700 | − | Transgenic |
| | | | | | insert |
| TI-1024 | TCCG | LbCas12a-TYC | 8681:8703 | − | Transgenic |
| | | | | | insert |
| TI-1025 | CTTC | FnCas12a | 8722:8744 | + | Transgenic |
| | | | | | insert |
| TI-1026 | TTCA | LbCas12a-TYC | 8701:8723 | − | Transgenic |
| | | | | | insert |
| TI-1027 | GTTC | FnCas12a | 8702:8724 | − | Transgenic |
| | | | | | insert |
| TI-1028 | TTCC | LbCas12a-TYC | 8742:8764 | + | Transgenic |
| | | | | | insert |
| TI-1029 | TCCC | LbCas12a-TYC | 8743:8765 | + | Transgenic |
| | | | | | insert |
| TI-1030 | TCCA | LbCas12a-TYC | 8725:8747 | − | Transgenic |
| | | | | | insert |
| TI-1031 | TTCC | LbCas12a-TYC | 8726:8748 | − | Transgenic |
| | | | | | insert |
| TI-1032 | TTTC | LbCas12a | 8727:8749 | − | Transgenic |
| | | | | | insert |
| TI-1033 | TCCA | LbCas12a-TYC | 8745:8767 | − | Transgenic |
| | | | | | insert |
| TI-1034 | TCCA | LbCas12a-TYC | 8756:8778 | − | Transgenic |
| | | | | | insert |
| TI-1035 | TCCA | LbCas12a-TYC | 8789:8811 | + | Transgenic |
| | | | | | insert |
| TI-1036 | TCCC | LbCas12a-TYC | 8804:8826 | + | Transgenic |
| | | | | | insert |
| TI-1037 | TATC | LbCas12a-TATV | 8813:8835 | + | Transgenic |
| | | | | | insert |
| TI-1038 | TCCC | LbCas12a-TYC | 8815:8837 | + | Transgenic |
| | | | | | insert |
| TI-1039 | TTTG | LbCas12a | 8823:8845 | + | Transgenic |
| | | | | | insert |
| TI-1040 | GTTG | FnCas12a | 8835:8857 | + | Transgenic |
| | | | | | insert |
| TI-1041 | TCCA | LbCas12a-TYC | 8840:8862 | + | Transgenic |
| | | | | | insert |
| TI-1042 | CTTA | FnCas12a | 8845:8867 | + | Transgenic |
| | | | | | insert |
| TI-1043 | TCCG | LbCas12a-TYC | 8831:8853 | − | Transgenic |
| | | | | | insert |
| TI-1044 | GTTA | FnCas12a | 8863:8885 | + | Transgenic |
| | | | | | insert |
| TI-1045 | TCCA | LbCas12a-TYC | 8848:8870 | − | Transgenic |
| | | | | | insert |
| TI-1046 | GTTG | FnCas12a | 8855:8877 | − | Transgenic |
| | | | | | insert |
| TI-1047 | CTTG | FnCas12a | 8861:8883 | − | Transgenic |
| | | | | | insert |
| TI-1048 | TCCA | LbCas12a-TYC | 8872:8894 | − | Transgenic |
| | | | | | insert |
| TI-1049 | CTTG | FnCas12a | 8895:8917 | + | Transgenic |
| | | | | | insert |
| TI-1050 | TATC | LbCas12a-TATV | 8874:8896 | − | Transgenic |
| | | | | | insert |
| TI-1051 | GTTA | FnCas12a | 8876:8898 | − | Transgenic |
| | | | | | insert |
| TI-1052 | TCCA | LbCas12a-TYC | 8884:8906 | − | Transgenic |
| | | | | | insert |
| TI-1053 | TCCA | LbCas12a-TYC | 8891:8913 | − | Transgenic |
| | | | | | insert |
| TI-1054 | CTTG | FnCas12a | 8916:8938 | + | Transgenic |
| | | | | | insert |
| TI-1055 | GTTA | FnCas12a | 8918:8940 | − | Transgenic |
| | | | | | insert |
| TI-1056 | TTTG | LbCas12a | 8925:8947 | − | Transgenic |
| | | | | | insert |
| TI-1057 | TATC | LbCas12a-TATV | 8940:8962 | − | Transgenic |
| | | | | | insert |
| TI-1058 | CTTC | FnCas12a | 8963:8985 | + | Transgenic |
| | | | | | insert |
| TI-1059 | TTCG | LbCas12a-TYC | 8964:8986 | + | Transgenic |
| | | | | | insert |
| TI-1060 | CTTG | FnCas12a | 8973:8995 | + | Transgenic |
| | | | | | insert |
| TI-1061 | GTTG | FnCas12a | 8991:9013 | + | Transgenic |
| | | | | | insert |
| TI-1062 | TCCA | LbCas12a-TYC | 9007:9029 | + | Transgenic |
| | | | | | insert |
| TI-1063 | TTCA | LbCas12a-TYC | 8992:9014 | − | Transgenic |
| | | | | | insert |
| TI-1064 | CTTC | FnCas12a | 8993:9015 | − | Transgenic |
| | | | | | insert |
| TI-1065 | TCCC | LbCas12a-TYC | 9025:9047 | + | Transgenic |
| | | | | | insert |
| TI-1066 | GTTC | FnCas12a | 9045:9067 | + | Transgenic |
| | | | | | insert |
| TI-1067 | CTTA | FnCas12a | 9029:9051 | − | Transgenic |
| | | | | | insert |
| TI-1068 | GTTC | FnCas12a | 9059:9081 | + | Transgenic |
| | | | | | insert |
| TI-1069 | CTTG | FnCas12a | 9062:9084 | + | Transgenic |
| | | | | | insert |
| TI-1070 | GTTA | FnCas12a | 9047:9069 | − | Transgenic |
| | | | | | insert |
| TI-1071 | GTTG | FnCas12a | 9084:9106 | + | Transgenic |
| | | | | | insert |
| TI-1072 | TCCA | LbCas12a-TYC | 9066:9088 | − | Transgenic |
| | | | | | insert |
| TI-1073 | TTCC | LbCas12a-TYC | 9067:9089 | − | Transgenic |
| | | | | | insert |
| TI-1074 | GTTC | FnCas12a | 9068:9090 | − | Transgenic |
| | | | | | insert |
| TI-1075 | TCCA | LbCas12a-TYC | 9081:9103 | − | Transgenic |
| | | | | | insert |
| TI-1076 | TCCG | LbCas12a-TYC | 9103:9125 | + | Transgenic |
| | | | | | insert |
| TI-1077 | TTCC | LbCas12a-TYC | 9082:9104 | − | Transgenic |
| | | | | | insert |
| TI-1078 | CTTA | FnCas12a | 9086:9108 | − | Transgenic |
| | | | | | insert |
| TI-1079 | TTCA | LbCas12a-TYC | 9103:9125 | − | Transgenic |
| | | | | | insert |
| TI-1080 | TATG | LbCas12a-TATV | 9125:9147 | + | Transgenic |
| | | | | | insert |
| TI-1081 | GTTC | FnCas12a | 9104:9126 | − | Transgenic |
| | | | | | insert |
| TI-1082 | CTTC | FnCas12a | 9134:9156 | + | Transgenic |
| | | | | | insert |
| TI-1083 | TTCA | LbCas12a-TYC | 9135:9157 | + | Transgenic |
| | | | | | insert |
| TI-1084 | TCCG | LbCas12a-TYC | 9124:9146 | − | Transgenic |
| | | | | | insert |
| TI-1085 | TTCC | LbCas12a-TYC | 9125:9147 | − | Transgenic |
| | | | | | insert |
| TI-1086 | CTTC | FnCas12a | 9126:9148 | − | Transgenic |
| | | | | | insert |
| TI-1087 | TCCC | LbCas12a-TYC | 9129:9151 | − | Transgenic |
| | | | | | insert |
| TI-1088 | TTCG | LbCas12a-TYC | 9136:9158 | − | Transgenic |
| | | | | | insert |
| TI-1089 | CTTC | FnCas12a | 9137:9159 | − | Transgenic |
| | | | | | insert |
| TI-1090 | TCCA | LbCas12a-TYC | 9178:9200 | + | Transgenic |
| | | | | | insert |
| TI-1091 | TCCC | LbCas12a-TYC | 9183:9205 | + | Transgenic |
| | | | | | insert |
| TI-1092 | TTCC | LbCas12a-TYC | 9162:9184 | − | Transgenic |
| | | | | | insert |
| TI-1093 | GTTC | FnCas12a | 9163:9185 | − | Transgenic |
| | | | | | insert |
| TI-1094 | TATG | LbCas12a-TATV | 9168:9190 | − | Transgenic |
| | | | | | insert |
| TI-1095 | TATC | LbCas12a-TATV | 9197:9219 | + | Transgenic |
| | | | | | insert |
| TI-1096 | CTTA | FnCas12a | 9201:9223 | + | Transgenic |
| | | | | | insert |
| TI-1097 | CTTG | FnCas12a | 9193:9215 | − | Transgenic |
| | | | | | insert |
| TI-1098 | CTTC | FnCas12a | 9233:9255 | + | Transgenic |
| | | | | | insert |
| TI-1099 | CTTC | FnCas12a | 9236:9258 | + | Transgenic |
| | | | | | insert |
| TI-1100 | TTCG | LbCas12a-TYC | 9237:9259 | + | Transgenic |
| | | | | | insert |
| TI-1101 | GTTC | FnCas12a | 9244:9266 | + | Transgenic |
| | | | | | insert |
| TI-1102 | TTCC | LbCas12a-TYC | 9245:9267 | + | Transgenic |
| | | | | | insert |
| TI-1103 | GTTG | FnCas12a | 9224:9246 | − | Transgenic |
| | | | | | insert |
| TI-1104 | TCCC | LbCas12a-TYC | 9246:9268 | + | Transgenic |
| | | | | | insert |
| TI-1105 | CTTC | FnCas12a | 9254:9276 | + | Transgenic |
| | | | | | insert |
| TI-1106 | TTCG | LbCas12a-TYC | 9255:9277 | + | Transgenic |
| | | | | | insert |
| TI-1107 | TATA | LbCas12a-TATV | 9233:9255 | − | Transgenic |
| | | | | | insert |
| TI-1108 | TATA | LbCas12a-TATV | 9260:9282 | + | Transgenic |
| | | | | | insert |
| TI-1109 | TCCA | LbCas12a-TYC | 9269:9291 | + | Transgenic |
| | | | | | insert |
| TI-1110 | TCCA | LbCas12a-TYC | 9250:9272 | − | Transgenic |
| | | | | | insert |
| TI-1111 | GTTC | FnCas12a | 9285:9307 | + | Transgenic |
| | | | | | insert |
| TI-1112 | CTTG | FnCas12a | 9273:9295 | − | Transgenic |
| | | | | | insert |
| TI-1113 | CTTG | FnCas12a | 9279:9301 | − | Transgenic |
| | | | | | insert |
| TI-1114 | TTCA | LbCas12a-TYC | 9289:9311 | − | Transgenic |
| | | | | | insert |
| TI-1115 | CTTC | FnCas12a | 9290:9312 | − | Transgenic |
| | | | | | insert |
| TI-1116 | TTTG | LbCas12a | 9299:9321 | − | Transgenic |
| | | | | | insert |
| TI-1117 | TTCA | LbCas12a-TYC | 9311:9333 | − | Transgenic |
| | | | | | insert |
| TI-1118 | CTTC | FnCas12a | 9312:9334 | − | Transgenic |
| | | | | | insert |
| TI-1119 | GTTG | FnCas12a | 9336:9358 | + | Transgenic |
| | | | | | insert |
| TI-1120 | TTCG | LbCas12a-TYC | 9320:9342 | − | Transgenic |
| | | | | | insert |
| TI-1121 | TTTC | LbCas12a | 9321:9343 | − | Transgenic |
| | | | | | insert |
| TI-1122 | TATC | LbCas12a-TATV | 9344:9366 | + | Transgenic |
| | | | | | insert |
| TI-1123 | TCCG | LbCas12a-TYC | 9327:9349 | − | Transgenic |
| | | | | | insert |
| TI-1124 | CTTA | FnCas12a | 9361:9383 | + | Transgenic |
| | | | | | insert |
| TI-1125 | TTCG | LbCas12a-TYC | 9341:9363 | − | Transgenic |
| | | | | | insert |
| TI-1126 | CTTC | FnCas12a | 9342:9364 | − | Transgenic |
| | | | | | insert |
| TI-1127 | TTCG | LbCas12a-TYC | 9346:9368 | − | Transgenic |
| | | | | | insert |
| TI-1128 | GTTC | FnCas12a | 9347:9369 | − | Transgenic |
| | | | | | insert |
| TI-1129 | TCCC | LbCas12a-TYC | 9351:9373 | − | Transgenic |
| | | | | | insert |
| TI-1130 | GTTG | FnCas12a | 9395:9417 | + | Transgenic |
| | | | | | insert |
| TI-1131 | TATC | LbCas12a-TATV | 9424:9446 | + | Transgenic |
| | | | | | insert |
| TI-1132 | TCCC | LbCas12a-TYC | 9403:9425 | − | Transgenic |
| | | | | | insert |
| TI-1133 | TCCA | LbCas12a-TYC | 9426:9448 | + | Transgenic |
| | | | | | insert |
| TI-1134 | CTTC | FnCas12a | 9413:9435 | − | Transgenic |
| | | | | | insert |
| TI-1135 | TTCG | LbCas12a-TYC | 9419:9441 | − | Transgenic |
| | | | | | insert |
| TI-1136 | GTTG | FnCas12a | 9423:9445 | − | Transgenic |
| | | | | | insert |
| TI-1137 | CTTC | FnCas12a | 9429:9451 | − | Transgenic |
| | | | | | insert |
| TI-1138 | GTTG | FnCas12a | 9451:9473 | − | Transgenic |
| | | | | | insert |
| TI-1139 | TCCA | LbCas12a-TYC | 9476:9498 | + | Transgenic |
| | | | | | insert |
| TI-1140 | TCCA | LbCas12a-TYC | 9456:9478 | − | Transgenic |
| | | | | | insert |
| TI-1141 | TCCC | LbCas12a-TYC | 9468:9490 | − | Transgenic |
| | | | | | insert |
| TI-1142 | GTTA | FnCas12a | 9477:9499 | − | Transgenic |
| | | | | | insert |
| TI-1143 | TATG | LbCas12a-TATV | 9480:9502 | − | Transgenic |
| | | | | | insert |
| TI-1144 | TTTA | LbCas12a | 9482:9504 | − | Transgenic |
| | | | | | insert |
| TI-1145 | TTTG | LbCas12a | 9523:9545 | + | Transgenic |
| | | | | | insert |
| TI-1146 | TTCA | LbCas12a-TYC | 9503:9525 | − | Transgenic |
| | | | | | insert |
| TI-1147 | GTTG | FnCas12a | 9513:9535 | − | Transgenic |
| | | | | | insert |
| TI-1148 | TTTC | LbCas12a | 9545:9567 | + | Transgenic |
| | | | | | insert |
| TI-1149 | CTTA | FnCas12a | 9530:9552 | − | Transgenic |
| | | | | | insert |
| TI-1150 | TTCA | LbCas12a-TYC | 9541:9563 | − | Transgenic |
| | | | | | insert |
| TI-1151 | GTTC | FnCas12a | 9542:9564 | − | Transgenic |
| | | | | | insert |
| TI-1152 | GTTG | FnCas12a | 9566:9588 | + | Transgenic |
| | | | | | insert |
| TI-1153 | CTTG | FnCas12a | 9572:9594 | + | Transgenic |
| | | | | | insert |
| TI-1154 | TTTA | LbCas12a | 9583:9605 | + | Transgenic |
| | | | | | insert |
| TI-1155 | TATG | LbCas12a-TATV | 9585:9607 | + | Transgenic |
| | | | | | insert |
| TI-1156 | TTTA | LbCas12a | 9567:9589 | − | Transgenic |
| | | | | | insert |
| TI-1157 | TTTG | LbCas12a | 9609:9631 | + | Transgenic |
| | | | | | insert |
| TI-1158 | GTTG | FnCas12a | 9615:9637 | + | Transgenic |
| | | | | | insert |
| TI-1159 | TATA | LbCas12a-TATV | 9609:9631 | − | Transgenic |
| | | | | | insert |
| TI-1160 | GTTA | FnCas12a | 9634:9656 | + | Transgenic |
| | | | | | insert |
| TI-1161 | TATA | LbCas12a-TATV | 9636:9658 | + | Transgenic |
| | | | | | insert |
| TI-1162 | TTCA | LbCas12a-TYC | 9619:9641 | − | Transgenic |
| | | | | | insert |
| TI-1163 | TATG | LbCas12a-TATV | 9641:9663 | + | Transgenic |
| | | | | | insert |
| TI-1164 | TTTC | LbCas12a | 9620:9642 | − | Transgenic |
| | | | | | insert |
| TI-1165 | TCCA | LbCas12a-TYC | 9625:9647 | − | Transgenic |
| | | | | | insert |
| TI-1166 | TATA | LbCas12a-TATV | 9642:9664 | − | Transgenic |
| | | | | | insert |
| TI-1167 | CTTA | FnCas12a | 9730:9752 | + | Transgenic |
| | | | | | insert |
| TI-1168 | TTCA | LbCas12a-TYC | 9719:9741 | − | Transgenic |
| | | | | | insert |
| TI-1169 | GTTC | FnCas12a | 9720:9742 | − | Transgenic |
| | | | | | insert |
| TI-1170 | TTTA | LbCas12a | 9729:9751 | − | Transgenic |
| | | | | | insert |
| TI-1171 | TTTA | LbCas12a | 9791:9813 | + | Transgenic |
| | | | | | insert |
| TI-1172 | TTTA | LbCas12a | 9819:9841 | + | Transgenic |
| | | | | | insert |
| TI-1173 | TATA | LbCas12a-TATV | 9840:9862 | + | Transgenic |
| | | | | | insert |
| TI-1174 | TTTC | LbCas12a | 9845:9867 | + | Transgenic |
| | | | | | insert |
| TI-1175 | TTCG | LbCas12a-TYC | 9846:9868 | + | Transgenic |
| | | | | | insert |
| TI-1176 | CTTG | FnCas12a | 9831:9853 | − | Transgenic |
| | | | | | insert |
| TI-1177 | TCCG | LbCas12a-TYC | 9868:9890 | + | Transgenic |
| | | | | | insert |
| TI-1178 | TCCG | LbCas12a-TYC | 9874:9896 | + | Transgenic |
| | | | | | insert |
| TI-1179 | GTTC | FnCas12a | 9883:9905 | + | Transgenic |
| | | | | | insert |
| TI-1180 | TTCG | LbCas12a-TYC | 9884:9906 | + | Transgenic |
| | | | | | insert |
| TI-1181 | TCCA | LbCas12a-TYC | 9895:9917 | + | Transgenic |
| | | | | | insert |
| TI-1182 | CTTA | FnCas12a | 9914:9936 | + | Transgenic |
| | | | | | insert |
| TI-1183 | TTTA | LbCas12a | 9905:9927 | − | Transgenic |
| | | | | | insert |
| TI-1184 | TTTG | LbCas12a | 9927:9949 | − | Transgenic |
| | | | | | insert |
| TI-1185 | GTTA | FnCas12a | 9964:9986 | + | Transgenic |
| | | | | | insert |
| TI-1186 | TTCC | LbCas12a-TYC | 9947:9969 | − | Transgenic |
| | | | | | insert |
| TI-1187 | TTTG | LbCas12a | 9960:9982 | − | Transgenic |
| | | | | | insert |
| TI-1188 | TCCA | LbCas12a-TYC | 9985:10007 | + | Transgenic |
| | | | | | insert |
| TI-1189 | GTTG | FnCas12a | 9968:9990 | − | Transgenic |
| | | | | | insert |
| TI-1190 | CTTG | FnCas12a | 9971:9993 | − | Transgenic |
| | | | | | insert |
| TI-1191 | TTTG | LbCas12a | 9993:10015 | − | Transgenic |
| | | | | | insert |
| TI-1192 | TCCA | LbCas12a-TYC | 10018:10040 | + | Transgenic |
| | | | | | insert |
| TI-1193 | TTTG | LbCas12a | 10005:10027 | − | Transgenic |
| | | | | | insert |
| TI-1194 | TTCA | LbCas12a-TYC | 10025:10047 | − | Transgenic |
| | | | | | insert |
| TI-1195 | CTTC | FnCas12a | 10026:10048 | − | Transgenic |
| | | | | | insert |
| TI-1196 | TTCA | LbCas12a-TYC | 10061:10083 | + | Transgenic |
| | | | | | insert |
| TI-1197 | TTTG | LbCas12a | 10040:10062 | − | Transgenic |
| | | | | | insert |
| TI-1198 | TTTA | LbCas12a | 10045:10067 | − | Transgenic |
| | | | | | insert |
| TI-1199 | GTTC | FnCas12a | 10081:10103 | + | Transgenic |
| | | | | | insert |
| TI-1200 | TTCC | LbCas12a-TYC | 10082:10104 | + | Transgenic |
| | | | | | insert |
| TI-1201 | TCCA | LbCas12a-TYC | 10083:10105 | + | Transgenic |
| | | | | | insert |
| TI-1202 | CTTA | FnCas12a | 10079:10101 | − | Transgenic |
| | | | | | insert |
| TI-1203 | TTTC | LbCas12a | 10088:10110 | − | Transgenic |
| | | | | | insert |
| TI-1204 | TCCA | LbCas12a-TYC | 10125:10147 | + | Transgenic |
| | | | | | insert |
| TI-1205 | CTTA | FnCas12a | 10136:10158 | + | Transgenic |
| | | | | | insert |
| TI-1206 | GTTA | FnCas12a | 10142:10164 | + | Transgenic |
| | | | | | insert |
| TI-1207 | TTCA | LbCas12a-TYC | 10130:10152 | − | Transgenic |
| | | | | | insert |
| TI-1208 | TTTC | LbCas12a | 10131:10153 | − | Transgenic |
| | | | | | insert |
| TI-1209 | TTTG | LbCas12a | 10155:10177 | + | Transgenic |
| | | | | | insert |
| TI-1210 | GTTG | FnCas12a | 10145:10167 | − | Transgenic |
| | | | | | insert |
| TI-1211 | CTTG | FnCas12a | 10167:10189 | + | Transgenic |
| | | | | | insert |
| TI-1212 | TCCC | LbCas12a-TYC | 10168:10190 | − | Transgenic |
| | | | | | insert |
| TI-1213 | TTTG | LbCas12a | 10177:10199 | − | Transgenic |
| | | | | | insert |
| TI-1214 | GTTA | FnCas12a | 10227:10249 | + | Transgenic |
| | | | | | insert |
| TI-1215 | TTTG | LbCas12a | 10215:10237 | − | Transgenic |
| | | | | | insert |
| TI-1216 | TTTG | LbCas12a | 10252:10274 | + | Transgenic |
| | | | | | insert |
| TI-1217 | TTTA | LbCas12a | 10258:10280 | + | Transgenic |
| | | | | | insert |
| TI-1218 | TTTG | LbCas12a | 10238:10260 | − | Transgenic |
| | | | | | insert |
| TI-1219 | TATC | LbCas12a-TATV | 10242:10264 | − | Transgenic |
| | | | | | insert |
| TI-1220 | TTTA | LbCas12a | 10244:10266 | − | Transgenic |
| | | | | | insert |
| TI-1221 | TTCC | LbCas12a-TYC | 10280:10302 | + | Transgenic |
| | | | | | insert |
| TI-1222 | CTTG | FnCas12a | 10264:10286 | − | Transgenic |
| | | | | | insert |
| TI-1223 | TCCC | LbCas12a-TYC | 10270:10292 | − | Transgenic |
| | | | | | insert |
| TI-1224 | TTCC | LbCas12a-TYC | 10271:10293 | − | Transgenic |
| | | | | | insert |
| TI-1225 | GTTC | FnCas12a | 10272:10294 | − | Transgenic |
| | | | | | insert |
| TI-1226 | TTTG | LbCas12a | 10275:10297 | − | Transgenic |
| | | | | | insert |
| TI-1227 | GTTA | FnCas12a | 10280:10302 | − | Transgenic |
| | | | | | insert |
| TI-1228 | TCCA | LbCas12a-TYC | 10285:10307 | − | Transgenic |
| | | | | | insert |
| TI-1229 | TTCC | LbCas12a-TYC | 10286:10308 | − | Transgenic |
| | | | | | insert |
| TI-1230 | TTTC | LbCas12a | 10287:10309 | − | Transgenic |
| | | | | | insert |
| TI-1231 | TTCG | LbCas12a-TYC | 10328:10350 | − | Transgenic |
| | | | | | insert |
| TI-1232 | TATG | LbCas12a-TATV | 10350:10372 | + | Transgenic |
| | | | | | insert |
| TI-1233 | GTTC | FnCas12a | 10329:10351 | − | Transgenic |
| | | | | | insert |
| TI-1234 | TTTG | LbCas12a | 10346:10368 | − | Transgenic |
| | | | | | insert |
| TI-1235 | CTTG | FnCas12a | 10385:10407 | + | Transgenic |
| | | | | | insert |
| TI-1236 | TTTA | LbCas12a | 10398:10420 | + | Transgenic |
| | | | | | insert |
| TI-1237 | TTCC | LbCas12a-TYC | 10408:10430 | + | Transgenic |
| | | | | | insert |
| TI-1238 | TCCA | LbCas12a-TYC | 10409:10431 | + | Transgenic |
| | | | | | insert |
| TI-1239 | GTTC | FnCas12a | 10419:10441 | + | Transgenic |
| | | | | | insert |
| TI-1240 | TTCA | LbCas12a-TYC | 10420:10442 | + | Transgenic |
| | | | | | insert |
| TI-1241 | GTTG | FnCas12a | 10399:10421 | − | Transgenic |
| | | | | | insert |
| TI-1242 | CTTG | FnCas12a | 10402:10424 | − | Transgenic |
| | | | | | insert |
| TI-1243 | TATG | LbCas12a-TATV | 10414:10436 | − | Transgenic |
| | | | | | insert |
| TI-1244 | TATC | LbCas12a-TATV | 10443:10465 | + | Transgenic |
| | | | | | insert |
| TI-1245 | TTTA | LbCas12a | 10449:10471 | + | Transgenic |
| | | | | | insert |
| TI-1246 | TTTG | LbCas12a | 10428:10450 | − | Transgenic |
| | | | | | insert |
| TI-1247 | TTCA | LbCas12a-TYC | 10453:10475 | + | Transgenic |
| | | | | | insert |
| TI-1248 | TATC | LbCas12a-TATV | 10463:10485 | + | Transgenic |
| | | | | | insert |
| TI-1249 | TTTG | LbCas12a | 10442:10464 | − | Transgenic |
| | | | | | insert |
| TI-1250 | CTTG | FnCas12a | 10448:10470 | − | Transgenic |
| | | | | | insert |
| TI-1251 | CTTC | FnCas12a | 10451:10473 | − | Transgenic |
| | | | | | insert |
| TI-1252 | TTCC | LbCas12a-TYC | 10454:10476 | − | Transgenic |
| | | | | | insert |
| TI-1253 | GTTC | FnCas12a | 10455:10477 | − | Transgenic |
| | | | | | insert |
| TI-1254 | TCCC | LbCas12a-TYC | 10486:10508 | + | Transgenic |
| | | | | | insert |
| TI-1255 | TTTG | LbCas12a | 10468:10490 | − | Transgenic |
| | | | | | insert |
| TI-1256 | CTTA | FnCas12a | 10478:10500 | − | Transgenic |
| | | | | | insert |
| TI-1257 | TTTG | LbCas12a | 10501:10523 | + | Transgenic |
| | | | | | insert |
| TI-1258 | TTCC | LbCas12a-TYC | 10481:10503 | − | Transgenic |
| | | | | | insert |
| TI-1259 | CTTC | FnCas12a | 10482:10504 | − | Transgenic |
| | | | | | insert |
| TI-1260 | TATC | LbCas12a-TATV | 10491:10513 | − | Transgenic |
| | | | | | insert |
| TI-1261 | TTCG | LbCas12a-TYC | 10515:10537 | + | Transgenic |
| | | | | | insert |
| TI-1262 | CTTG | FnCas12a | 10496:10518 | − | Transgenic |
| | | | | | insert |
| TI-1263 | TATC | LbCas12a-TATV | 10520:10542 | + | Transgenic |
| | | | | | insert |
| TI-1264 | TATC | LbCas12a-TATV | 10503:10525 | − | Transgenic |
| | | | | | insert |
| TI-1265 | CTTG | FnCas12a | 10527:10549 | + | Transgenic |
| | | | | | insert |
| TI-1266 | TCCG | LbCas12a-TYC | 10508:10530 | − | Transgenic |
| | | | | | insert |
| TI-1267 | TTCC | LbCas12a-TYC | 10509:10531 | − | Transgenic |
| | | | | | insert |
| TI-1268 | CTTC | FnCas12a | 10510:10532 | − | Transgenic |
| | | | | | insert |
| TI-1269 | TATC | LbCas12a-TATV | 10532:10554 | + | Transgenic |
| | | | | | insert |
| TI-1270 | TTTC | LbCas12a | 10541:10563 | + | Transgenic |
| | | | | | insert |
| TI-1271 | CTTG | FnCas12a | 10548:10570 | + | Transgenic |
| | | | | | insert |
| TI-1272 | TCCC | LbCas12a-TYC | 10526:10548 | − | Transgenic |
| | | | | | insert |
| TI-1273 | GTTG | FnCas12a | 10558:10580 | + | Transgenic |
| | | | | | insert |
| TI-1274 | TCCA | LbCas12a-TYC | 10539:10561 | − | Transgenic |
| | | | | | insert |
| TI-1275 | TTCC | LbCas12a-TYC | 10540:10562 | − | Transgenic |
| | | | | | insert |
| TI-1276 | TCCC | LbCas12a-TYC | 10545:10567 | − | Transgenic |
| | | | | | insert |
| TI-1277 | TATG | LbCas12a-TATV | 10551:10573 | − | Transgenic |
| | | | | | insert |
| TI-1278 | TATG | LbCas12a-TATV | 10580:10602 | + | Transgenic |
| | | | | | insert |
| TI-1279 | GTTG | FnCas12a | 10584:10606 | + | Transgenic |
| | | | | | insert |
| TI-1280 | TATA | LbCas12a-TATV | 10562:10584 | − | Transgenic |
| | | | | | insert |
| TI-1281 | GTTA | FnCas12a | 10587:10609 | + | Transgenic |
| | | | | | insert |
| TI-1282 | TTTA | LbCas12a | 10567:10589 | − | Transgenic |
| | | | | | insert |
| TI-1283 | TATA | LbCas12a-TATV | 10589:10611 | + | Transgenic |
| | | | | | insert |
| TI-1284 | TTTC | LbCas12a | 10601:10623 | + | Transgenic |
| | | | | | insert |
| TI-1285 | TTCC | LbCas12a-TYC | 10602:10624 | + | Transgenic |
| | | | | | insert |
| TI-1286 | TCCA | LbCas12a-TYC | 10603:10625 | + | Transgenic |
| | | | | | insert |
| TI-1287 | TTTG | LbCas12a | 10619:10641 | + | Transgenic |
| | | | | | insert |
| TI-1288 | GTTA | FnCas12a | 10625:10647 | + | Transgenic |
| | | | | | insert |
| TI-1289 | TATA | LbCas12a-TATV | 10603:10625 | − | Transgenic |
| | | | | | insert |
| TI-1290 | TATA | LbCas12a-TATV | 10605:10627 | − | Transgenic |
| | | | | | insert |
| TI-1291 | TTTA | LbCas12a | 10637:10659 | + | Transgenic |
| | | | | | insert |
| TI-1292 | TTTA | LbCas12a | 10653:10675 | + | Transgenic |
| | | | | | insert |
| TI-1293 | TTTG | LbCas12a-TYC | 10657:10679 | + | Transgenic |
| | | | | | insert |
| TI-1294 | TTCA | LbCas12a-TYC | 10639:10661 | − | Transgenic |
| | | | | | insert |
| TI-1295 | TTTG | LbCas12a | 10664:10686 | + | Transgenic |
| | | | | | insert |
| TI-1296 | TTTA | LbCas12a-TYC | 10643:10665 | − | Transgenic |
| | | | | | insert |
| TI-1297 | TATA | LbCas12a-TATV | 10650:10672 | − | Transgenic |
| | | | | | insert |
| TI-1298 | TATA | LbCas12a-TATV | 10677:10699 | + | Transgenic |
| | | | | | insert |
| TI-1299 | TATC | LbCas12a-TATV | 10664:10686 | − | Transgenic |
| | | | | | insert |
| TI-1300 | CTTG | FnCas12a | 10688:10710 | + | Transgenic |
| | | | | | insert |
| TI-1301 | TTCA | LbCas12a-TYC | 10702:10724 | + | Transgenic |
| | | | | | insert |
| TI-1302 | TATC | LbCas12a-TATV | 10683:10705 | − | Transgenic |
| | | | | | insert |
| TI-1303 | TTTG | LbCas12a | 10688:10710 | − | Transgenic |
| | | | | | insert |
| TI-1304 | TATC | LbCas12a-TATV | 10712:10734 | + | Transgenic |
| | | | | | insert |
| TI-1305 | TTTA | LbCas12a | 10721:10743 | + | Transgenic |
| | | | | | insert |
| TI-1306 | CTTC | FnCas12a | 10705:10727 | − | Transgenic |
| | | | | | insert |
| TI-1307 | TATG | LbCas12a-TATV | 10714:10736 | − | Transgenic |
| | | | | | insert |
| TI-1308 | TATA | LbCas12a-TATV | 10716:10738 | − | Transgenic |
| | | | | | insert |
| TI-1309 | TATA | LbCas12a-TATV | 10743:10765 | + | Transgenic |
| | | | | | insert |
| TI-1310 | TTCA | LbCas12a-TYC | 10740:10762 | − | Transgenic |
| | | | | | insert |
| TI-1311 | TTTC | LbCas12a | 10741:10763 | − | Transgenic |
| | | | | | insert |
| TI-1312 | TTTG | LbCas12a | 10774:10796 | + | Transgenic |
| | | | | | insert |
| TI-1313 | TTTG | LbCas12a | 10799:10821 | + | Transgenic |
| | | | | | insert |
| TI-1314 | TTCA | LbCas12a-TYC | 10778:10800 | − | Transgenic |
| | | | | | insert |
| TI-1315 | CTTG | FnCas12a | 10803:10825 | + | Transgenic |
| | | | | | insert |
| TI-1316 | GTTA | FnCas12a | 10782:10804 | − | Transgenic |
| | | | | | insert |
| TI-1317 | CTTA | FnCas12a | 10813:10835 | + | Transgenic |
| | | | | | insert |
| TI-1318 | TTTA | LbCas12a | 10792:10814 | − | Transgenic |
| | | | | | insert |
| TI-1319 | TTTA | LbCas12a | 10805:10827 | − | Transgenic |
| | | | | | insert |
| TI-1320 | GTTA | FnCas12a | 10818:10840 | − | Transgenic |
| | | | | | insert |
| TI-1321 | TTTG | LbCas12a-TYC | 10841:10863 | + | Transgenic |
| | | | | | insert |
| TI-1322 | TTTA | LbCas12a | 10831:10853 | − | Transgenic |
| | | | | | insert |
| TI-1323 | TATG | LbCas12a-TATV | 10839:10861 | − | Transgenic |
| | | | | | insert |
| TI-1324 | TTTA | LbCas12a | 10841:10863 | − | Transgenic |
| | | | | | insert |
| TI-1325 | TTCA | LbCas12a-TYC | 10864:10886 | + | Transgenic |
| | | | | | insert |
| TI-1326 | GTTG | FnCas12a | 10848:10870 | − | Transgenic |
| | | | | | insert |
| TI-1327 | GTTG | FnCas12a | 10852:10874 | − | Transgenic |
| | | | | | insert |
| TI-1328 | CTTG | FnCas12a | 10857:10879 | − | Transgenic |
| | | | | | insert |
| TI-1329 | TTCA | LbCas12a-TYC | 10900:10922 | + | Transgenic |
| | | | | | insert |
| TI-1330 | TTTA | LbCas12a | 10881:10903 | − | Transgenic |
| | | | | | insert |
| TI-1331 | TATG | LbCas12a-TATV | 10891:10913 | − | Transgenic |
| | | | | | insert |
| TI-1332 | TTTA | LbCas12a | 10904:10926 | − | Transgenic |
| | | | | | insert |
| TI-1333 | TATC | LbCas12a-TATV | 10912:10934 | − | Transgenic |
| | | | | | insert |
| TI-1334 | TTTA | LbCas12a | 10947:10969 | + | Transgenic |
| | | | | | insert |
| TI-1335 | TTTA | LbCas12a-TYC | 10927:10949 | − | Transgenic |
| | | | | | insert |
| TI-1336 | TATG | LbCas12a-TATV | 10949:10971 | + | Transgenic |
| | | | | | insert |
| TI-1337 | GTTA | FnCas12a | 10952:10974 | + | Transgenic |
| | | | | | insert |
| TI-1338 | GTTA | FnCas12a | 10934:10956 | − | Transgenic |
| | | | | | insert |
| TI-1339 | TCCG | LbCas12a-TYC | 10946:10968 | − | Transgenic |
| | | | | | insert |
| TI-1340 | TTTC | LbCas12a | 10970:10992 | + | Transgenic |
| | | | | | insert |
| TI-1341 | TTCG | LbCas12a-TYC | 10971:10993 | + | Transgenic |
| | | | | | insert |
| TI-1342 | GTTG | FnCas12a | 10952:10974 | − | Transgenic |
| | | | | | insert |
| TI-1343 | TTCA | LbCas12a-TYC | 10977:10999 | + | Transgenic |
| | | | | | insert |
| TI-1344 | TATA | LbCas12a-TATV | 10958:10980 | − | Transgenic |
| | | | | | insert |
| TI-1345 | TTTA | LbCas12a | 10960:10982 | − | Transgenic |
| | | | | | insert |
| TI-1346 | TATA | LbCas12a-TATV | 10985:11007 | + | Transgenic |
| | | | | | insert |
| TI-1347 | TTTA | LbCas12a | 10964:10986 | − | Transgenic |
| | | | | | insert |
| TI-1348 | GTTG | FnCas12a | 11004:11026 | + | Transgenic |
| | | | | | insert |
| TI-1349 | TTCC | LbCas12a-TYC | 11012:11034 | + | Transgenic |
| | | | | | insert |
| TI-1350 | TCCA | LbCas12a-TYC | 11013:11035 | + | Transgenic |
| | | | | | insert |
| TI-1351 | TTTA | LbCas12a | 11017:11039 | + | Transgenic |
| | | | | | insert |
| TI-1352 | TATC | LbCas12a-TATV | 11019:11041 | + | Transgenic |
| | | | | | insert |
| TI-1353 | GTTC | FnCas12a | 11023:11045 | + | Transgenic |
| | | | | | insert |
| TI-1354 | CTTA | FnCas12a | 11026:11048 | + | Transgenic |
| | | | | | insert |
| TI-1355 | TTTC | LbCas12a | 11052:11074 | + | Transgenic |
| | | | | | insert |
| TI-1356 | TATC | LbCas12a-TATV | 11032:11054 | − | Transgenic |
| | | | | | insert |
| TI-1357 | TATC | LbCas12a-TATV | 11061:11083 | + | Transgenic |
| | | | | | insert |
| TI-1358 | CTTA | FnCas12a | 11045:11067 | − | Transgenic |
| | | | | | insert |
| TI-1359 | GTTA | FnCas12a | 11052:11074 | − | Transgenic |
| | | | | | insert |
| TI-1360 | GTTA | FnCas12a | 11077:11099 | + | Transgenic |
| | | | | | insert |
| TI-1361 | TTTC | LbCas12a | 11108:11130 | + | Transgenic |
| | | | | | insert |
| TI-1362 | TTCG | LbCas12a-TYC | 11109:11131 | + | Transgenic |
| | | | | | insert |
| TI-1363 | GTTG | FnCas12a | 11123:11145 | + | Transgenic |
| | | | | | insert |
| TI-1364 | TTTC | LbCas12a | 11136:11158 | + | Transgenic |
| | | | | | insert |
| TI-1365 | TTCG | LbCas12a-TYC | 11137:11159 | + | Transgenic |
| | | | | | insert |
| TI-1366 | TCCA | LbCas12a-TYC | 11122:11144 | − | Transgenic |
| | | | | | insert |
| TI-1367 | GTTG | FnCas12a | 11147:11169 | + | Transgenic |
| | | | | | insert |
| TI-1368 | TTCG | LbCas12a-TYC | 11153:11175 | + | Transgenic |
| | | | | | insert |
| TI-1369 | TTTG | LbCas12a | 11163:11185 | + | Transgenic |
| | | | | | insert |
| TI-1370 | GTTG | FnCas12a | 11166:11188 | + | Transgenic |
| | | | | | insert |
| TI-1371 | TTCA | LbCas12a-TYC | 11154:11176 | − | Transgenic |
| | | | | | insert |
| TI-1372 | TTTC | LbCas12a | 11155:11177 | − | Transgenic |
| | | | | | insert |
| TI-1373 | GTTC | FnCas12a | 11177:11199 | + | Transgenic |
| | | | | | insert |
| TI-1374 | TTTA | LbCas12a | 11200:11222 | + | Transgenic |
| | | | | | insert |
| TI-1375 | TTTA | LbCas12a | 11207:11229 | + | Transgenic |
| | | | | | insert |
| TI-1376 | TATC | LbCas12a-TATV | 11209:11231 | + | Transgenic |
| | | | | | insert |
| TI-1377 | GTTA | FnCas12a | 11217:11239 | + | Transgenic |
| | | | | | insert |
| TI-1378 | GTTG | FnCas12a | 11198:11220 | − | Transgenic |
| | | | | | insert |
| TI-1379 | TATC | LbCas12a-TATV | 11222:11244 | + | Transgenic |
| | | | | | insert |
| TI-1380 | TTTC | LbCas12a | 11230:11252 | + | Transgenic |
| | | | | | insert |
| TI-1381 | TTCA | LbCas12a-TYC | 11231:11253 | + | Transgenic |
| | | | | | insert |
| TI-1382 | TTTA | LbCas12a-TYC | 11215:11237 | − | Transgenic |
| | | | | | insert |
| TI-1383 | CTTC | FnCas12a | 11238:11260 | + | Transgenic |
| | | | | | insert |
| TI-1384 | TATC | LbCas12a-TATV | 11220:11242 | − | Transgenic |
| | | | | | insert |
| TI-1385 | TTTA | LbCas12a | 11252:11274 | + | Transgenic |
| | | | | | insert |
| TI-1386 | TTCA | LbCas12a-TYC | 11233:11255 | − | Transgenic |
| | | | | | insert |
| TI-1387 | TTTC | FnCas12a | 11234:11256 | − | Transgenic |
| | | | | | insert |
| TI-1388 | TTCA | LbCas12a-TYC | 11256:11278 | + | Transgenic |
| | | | | | insert |
| TI-1389 | TTTC | LbCas12a | 11269:11291 | + | Transgenic |
| | | | | | insert |
| TI-1390 | TTCC | LbCas12a-TYC | 11270:11292 | + | Transgenic |
| | | | | | insert |
| TI-1391 | TCCC | LbCas12a-TYC | 11271:11293 | + | Transgenic |
| | | | | | insert |
| TI-1392 | TTTG | LbCas12a | 11292:11314 | + | Transgenic |
| | | | | | insert |
| TI-1393 | TTTA | LbCas12a | 11300:11322 | + | Transgenic |
| | | | | | insert |
| TI-1394 | TTTG | LbCas12a | 11305:11327 | + | Transgenic |
| | | | | | insert |
| TI-1395 | GTTC | FnCas12a | 11308:11330 | + | Transgenic |
| | | | | | insert |
| TI-1396 | TTCG | LbCas12a-TYC | 11309:11331 | + | Transgenic |
| | | | | | insert |
| TI-1397 | GTTG | FnCas12a | 11320:11342 | + | Transgenic |
| | | | | | insert |
| TI-1398 | GTTG | FnCas12a | 11324:11346 | + | Transgenic |
| | | | | | insert |
| TI-1399 | TCCA | LbCas12a-TYC | 11302:11324 | − | Transgenic |
| | | | | | insert |
| TI-1400 | TATA | LbCas12a-TATV | 11308:11330 | − | Transgenic |
| | | | | | insert |
| TI-1401 | TATA | LbCas12a-TATV | 11310:11332 | | Transgenic |
| | | | | | insert |
| TI-1402 | TATA | LbCas12a-TATV | 11335:11357 | + | Transgenic |
| | | | | | insert |
| TI-1403 | TATA | LbCas12a-TATV | 11337:11359 | + | Transgenic |
| | | | | | insert |
| TI-1404 | TTCA | LbCas12a-TYC | 11318:11340 | − | Transgenic |
| | | | | | insert |
| TI-1405 | GTTC | FnCas12a | 11319:11341 | − | Transgenic |
| | | | | | insert |
| TI-1406 | CTTG | FnCas12a | 11349:11371 | + | Transgenic |
| | | | | | insert |
| TI-1407 | GTTG | FnCas12a | 11352:11374 | + | Transgenic |
| | | | | | insert |
| TI-1408 | TTTG | LbCas12a | 11357:11379 | + | Transgenic |
| | | | | | insert |
| TI-1409 | CTTA | FnCas12a | 11340:11362 | − | Transgenic |
| | | | | | insert |
| TI-1410 | TTTA | LbCas12a | 11365:11387 | + | Transgenic |
| | | | | | insert |
| TI-1411 | TATG | LbCas12a-TATV | 11374:11396 | + | Transgenic |
| | | | | | insert |
| TI-1412 | TTCA | LbCas12a-TYC | 11357:11379 | − | Transgenic |
| | | | | | insert |
| TI-1413 | TTTG | LbCas12a | 11382:11404 | + | Transgenic |
| | | | | | insert |
| TI-1414 | TCCA | LbCas12a-TYC | 11378:11400 | − | Transgenic |
| | | | | | insert |
| TI-1415 | TATG | LbCas12a-TATV | 11386:11408 | − | Transgenic |
| | | | | | insert |
| TI-1416 | GTTA | FnCas12a | 11388:11410 | − | Transgenic |
| | | | | | insert |
| TI-1417 | TTTG | LbCas12a | 11391:11413 | − | Transgenic |
| | | | | | insert |
| TI-1418 | TTTC | LbCas12a | 11436:11458 | + | Transgenic |
| | | | | | insert |
| TI-1419 | TTTG | LbCas12a | 11460:11482 | + | Transgenic |
| | | | | | insert |
| TI-1420 | TATA | LbCas12a-TATV | 11440:11462 | − | Transgenic |
| | | | | | insert |
| TI-1421 | TTTA | LbCas12a | 11465:11487 | + | Transgenic |
| | | | | | insert |
| TI-1422 | TATA | LbCas12a-TATV | 11467:11489 | + | Transgenic |
| | | | | | insert |
| TI-1423 | TATC | LbCas12a-TATV | 11469:11491 | + | Transgenic |
| | | | | | insert |
| TI-1424 | TTCA | LbCas12a-TYC | 11449:11471 | − | Transgenic |
| | | | | | insert |
| TI-1425 | TTTC | LbCas12a | 11450:11472 | − | Transgenic |
| | | | | | insert |
| TI-1426 | CTTG | FnCas12a | 11474:11496 | + | Transgenic |
| | | | | | insert |
| TI-1427 | TTTG | LbCas12a | 11488:11510 | + | Transgenic |
| | | | | | insert |
| TI-1428 | TTCA | LbCas12a-TYC | 11467:11489 | − | Transgenic |
| | | | | | insert |
| TI-1429 | TTTA | LbCas12a | 11498:11520 | + | Transgenic |
| | | | | | insert |
| TI-1430 | CTTA | FnCas12a | 11507:11529 | + | Transgenic |
| | | | | | insert |
| TI-1431 | TATC | LbCas12a-TATV | 11492:11514 | − | Transgenic |
| | | | | | insert |
| TI-1432 | TTTG | LbCas12a | 11536:11558 | + | Transgenic |
| | | | | | insert |
| TI-1433 | TATC | LbCas12a-TATV | 11535:11557 | − | Transgenic |
| | | | | | insert |
| TI-1434 | TTTA | LbCas12a | 11573:11595 | + | Transgenic |
| | | | | | insert |
| TI-1435 | TTTG | LbCas12a | 11587:11609 | + | Transgenic |
| | | | | | insert |
| TI-1436 | GTTA | FnCas12a | 11592:11614 | + | Transgenic |
| | | | | | insert |
| TI-1437 | TTTC | LbCas12a | 11604:11626 | + | Transgenic |
| | | | | | insert |
| TI-1438 | TTCA | LbCas12a-TYC | 11605:11627 | + | Transgenic |
| | | | | | insert |
| TI-1439 | TATA | LbCas12a-TATV | 11589:11611 | − | Transgenic |
| | | | | | insert |
| TI-1440 | TATA | LbCas12a-TATV | 11591:11613 | − | Transgenic |
| | | | | | insert |
| TI-1441 | TTTA | LbCas12a | 11633:11655 | + | Transgenic |
| | | | | | insert |
| TI-1442 | TTTG | LbCas12a | 11664:11686 | + | Transgenic |
| | | | | | insert |
| TI-1443 | TTTG | LbCas12a | 11672:11694 | + | Transgenic |
| | | | | | insert |
| TI-1444 | GTTG | FnCas12a | 11675:11697 | + | Transgenic |
| | | | | | insert |
| TI-1445 | TCCC | LbCas12a-TYC | 11711:11733 | + | Transgenic |
| | | | | | insert |
| TI-1446 | CTTG | FnCas12a | 11697:11719 | − | Transgenic |
| | | | | | insert |
| TI-1447 | TCCG | LbCas12a-TYC | 11721:11743 | − | Transgenic |
| | | | | | insert |
| TI-1448 | CTTC | FnCas12a | 11755:11777 | + | Transgenic |
| | | | | | insert |
| TI-1449 | TTCC | LbCas12a-TYC | 11756:11778 | + | Transgenic |
| | | | | | insert |
| TI-1450 | TCCG | LbCas12a-TYC | 11757:11779 | + | Transgenic |
| | | | | | insert |
| TI-1451 | TCCC | LbCas12a-TYC | 11764:11786 | + | Transgenic |
| | | | | | insert |
| TI-1452 | CTTC | FnCas12a | 11770:11792 | + | Transgenic |
| | | | | | insert |
| TI-1453 | TCCC | LbCas12a-TYC | 11749:11771 | − | Transgenic |
| | | | | | insert |
| TI-1454 | TTCC | LbCas12a-TYC | 11771:11793 | + | Transgenic |
| | | | | | insert |
| TI-1455 | TCCA | LbCas12a-TYC | 11772:11794 | + | Transgenic |
| | | | | | insert |
| TI-1456 | TCCG | LbCas12a-TYC | 11762:11784 | − | Transgenic |
| | | | | | insert |
| TI-1457 | TTCC | LbCas12a-TYC | 11763:11785 | − | Transgenic |
| | | | | | insert |
| TI-1458 | GTTC | FnCas12a | 11764:11786 | − | Transgenic |
| | | | | | insert |
| TI-1459 | TCCC | LbCas12a-TYC | 11786:11808 | − | Transgenic |
| | | | | | insert |
| TI-1460 | TCCG | LbCas12a-TYC | 11834:11856 | + | Transgenic |
| | | | | | insert |
| TI-1461 | CTTG | FnCas12a | 11818:11840 | − | Transgenic |
| | | | | | insert |
| TI-1462 | TCCC | LbCas12a-TYC | 11842:11864 | + | Transgenic |
| | | | | | insert |
| TI-1463 | TTCC | LbCas12a-TYC | 11829:11851 | − | Transgenic |
| | | | | | insert |
| TI-1464 | CTTC | FnCas12a | 11830:11852 | − | Transgenic |
| | | | | | insert |
| TI-1465 | TTTC | LbCas12a | 11869:11891 | + | Transgenic |
| | | | | | insert |
| TI-1466 | TTCG | LbCas12a-TYC | 11870:11892 | + | Transgenic |
| | | | | | insert |
| TI-1467 | CTTG | FnCas12a | 11884:11906 | + | Transgenic |
| | | | | | insert |
| TI-1468 | CTTC | FnCas12a | 11906:11928 | + | Transgenic |
| | | | | | insert |
| TI-1469 | TTCA | LbCas12a-TYC | 11907:11929 | + | Transgenic |
| | | | | | insert |
| TI-1470 | CTTC | FnCas12a | 11916:11938 | + | Transgenic |
| | | | | | insert |
| TI-1471 | CTTC | FnCas12a | 11919:11941 | + | Transgenic |
| | | | | | insert |
| TI-1472 | TTCC | LbCas12a-TYC | 11920:11942 | + | Transgenic |
| | | | | | insert |
| TI-1473 | TCCA | LbCas12a-TYC | 11921:11943 | + | Transgenic |
| | | | | | insert |
| TI-1474 | GTTG | FnCas12a | 11904:11926 | − | Transgenic |
| | | | | | insert |
| TI-1475 | TTTC | LbCas12a | 11913:11935 | − | Transgenic |
| | | | | | insert |
| TI-1476 | GTTC | FnCas12a | 11946:11968 | + | Transgenic |
| | | | | | insert |
| TI-1477 | CTTG | FnCas12a | 11953:11975 | + | Transgenic |
| | | | | | insert |
| TI-1478 | TCCA | LbCas12a-TYC | 11932:11954 | − | Transgenic |
| | | | | | insert |
| TI-1479 | TTCC | LbCas12a-TYC | 11933:11955 | − | Transgenic |
| | | | | | insert |
| TI-1480 | GTTC | FnCas12a | 11934:11956 | − | Transgenic |
| | | | | | insert |
| TI-1481 | TCCG | LbCas12a-TYC | 11967:11989 | + | Transgenic |
| | | | | | insert |
| TI-1482 | TTCC | LbCas12a-TYC | 11973:11995 | + | Transgenic |
| | | | | | insert |
| TI-1483 | TCCA | LbCas12a-TYC | 11974:11996 | + | Transgenic |
| | | | | | insert |
| TI-1484 | TTTG | LbCas12a | 11956:11978 | − | Transgenic |
| | | | | | insert |
| TI-1485 | TTTC | LbCas12a | 12004:12026 | + | Transgenic |
| | | | | | insert |
| TI-1486 | TTCA | LbCas12a-TYC | 12005:12027 | + | Transgenic |
| | | | | | insert |
| TI-1487 | TTTG | LbCas12a | 12011:12033 | + | Transgenic |
| | | | | | insert |
| TI-1488 | TCCA | LbCas12a-TYC | 12001:12023 | − | Transgenic |
| | | | | | insert |
| TI-1489 | TCCA | LbCas12a-TYC | 12027:12049 | − | Transgenic |
| | | | | | insert |
| TI-1490 | CTTC | FnCas12a | 12050:12072 | + | Transgenic |
| | | | | | insert |
| TI-1491 | TTCC | LbCas12a-TYC | 12028:12050 | − | Transgenic |
| | | | | | insert |
| TI-1492 | CTTC | FnCas12a | 12029:12051 | − | Transgenic |
| | | | | | insert |
| TI-1493 | TCCC | LbCas12a-TYC | 12053:12075 | − | Transgenic |
| | | | | | insert |
| TI-1494 | TTCC | LbCas12a-TYC | 12054:12076 | − | Transgenic |
| | | | | | insert |
| TI-1495 | CTTC | FnCas12a | 12055:12077 | − | Transgenic |
| | | | | | insert |
| TI-1496 | TATG | LbCas12a-TATV | 12097:12119 | + | Transgenic |
| | | | | | insert |
| TI-1497 | CTTG | FnCas12a | 12088:12110 | − | Transgenic |
| | | | | | insert |
| TI-1498 | TTCG | LbCas12a-TYC | 12111:12133 | + | Transgenic |
| | | | | | insert |
| TI-1499 | TTCC | LbCas12a-TYC | 12091:12113 | − | Transgenic |
| | | | | | insert |
| TI-1500 | CTTC | FnCas12a | 12092:12114 | − | Transgenic |
| | | | | | insert |
| TI-1501 | TATC | LbCas12a-TATV | 12098:12120 | − | Transgenic |
| | | | | | insert |
| TI-1502 | TCCA | LbCas12a-TYC | 12104:12126 | − | Transgenic |
| | | | | | insert |
| TI-1503 | CTTG | FnCas12a | 12129:12151 | + | Transgenic |
| | | | | | insert |
| TI-1504 | TATG | LbCas12a-TATV | 12109:12131 | − | Transgenic |
| | | | | | insert |
| TI-1505 | TCCG | LbCas12a-TYC | 12115:12137 | − | Transgenic |
| | | | | | insert |
| TI-1506 | TCCC | LbCas12a-TYC | 12122:12144 | − | Transgenic |
| | | | | | insert |
| TI-1507 | TTCC | LbCas12a-TYC | 12123:12145 | − | Transgenic |
| | | | | | insert |
| TI-1508 | GTTC | FnCas12a | 12124:12146 | − | Transgenic |
| | | | | | insert |
| TI-1509 | GTTG | FnCas12a | 12146:12168 | + | Transgenic |
| | | | | | insert |
| TI-1510 | CTTA | FnCas12a | 12160:12182 | + | Transgenic |
| | | | | | insert |
| TI-1511 | CTTG | FnCas12a | 12164:12186 | + | Transgenic |
| | | | | | insert |
| TI-1512 | TCCG | LbCas12a-TYC | 12144:12166 | − | Transgenic |
| | | | | | insert |
| TI-1513 | TTTG | LbCas12a | 12188:12210 | + | Transgenic |
| | | | | | insert |
| TI-1514 | GTTG | FnCas12a | 12166:12188 | − | Transgenic |
| | | | | | insert |
| TI-1515 | CTTA | FnCas12a | 12215:12237 | + | Transgenic |
| | | | | | insert |
| TI-1516 | TATG | LbCas12a-TATV | 12220:12242 | + | Transgenic |
| | | | | | insert |
| TI-1517 | TTTA | LbCas12a | 12237:12259 | + | Transgenic |
| | | | | | insert |
| TI-1518 | TTTC | LbCas12a | 12244:12266 | + | Transgenic |
| | | | | | insert |
| TI-1519 | TTCG | LbCas12a-TYC | 12245:12267 | + | Transgenic |
| | | | | | insert |
| TI-1520 | GTTG | FnCas12a | 12225:12247 | − | Transgenic |
| | | | | | insert |
| TI-1521 | TTCA | LbCas12a-TYC | 12250:12272 | + | Transgenic |
| | | | | | insert |
| TI-1522 | TTTA | LbCas12a | 12257:12279 | + | Transgenic |
| | | | | | insert |
| TI-1523 | TCCC | LbCas12a-TYC | 12237:12259 | − | Transgenic |
| | | | | | insert |
| TI-1524 | TTTA | LbCas12a | 12253:12275 | − | Transgenic |
| | | | | | insert |
| TI-1525 | TCCC | LbCas12a-TYC | 12265:12287 | − | Transgenic |
| | | | | | insert |
| TI-1526 | TTCC | LbCas12a-TYC | 12266:12288 | − | Transgenic |
| | | | | | insert |
| TI-1527 | CTTC | FnCas12a | 12267:12289 | − | Transgenic |
| | | | | | insert |
| TI-1528 | GTTA | FnCas12a | 12277:12299 | − | Transgenic |
| | | | | | insert |
| TI-1529 | CTTA | FnCas12a | 12302:12324 | + | Transgenic |
| | | | | | insert |
| TI-1530 | GTTG | FnCas12a | 12310:12332 | + | Transgenic |
| | | | | | insert |
| TI-1531 | TCCC | LbCas12a-TYC | 12288:12310 | − | Transgenic |
| | | | | | insert |
| TI-1532 | CTTG | FnCas12a | 12302:12324 | − | Transgenic |
| | | | | | insert |
| TI-1533 | CTTA | FnCas12a | 12307:12329 | − | Transgenic |
| | | | | | insert |
| TI-1534 | GTTA | FnCas12a | 12332:12354 | + | Transgenic |
| | | | | | insert |
| TI-1535 | TCCG | LbCas12a-TYC | 12338:12360 | + | Transgenic |
| | | | | | insert |
| TI-1536 | CTTG | FnCas12a | 12367:12389 | + | Transgenic |
| | | | | | insert |
| TI-1537 | CTTA | FnCas12a | 12352:12374 | − | Transgenic |
| | | | | | insert |
| TI-1538 | CTTA | FnCas12a | 12399:12421 | + | Transgenic |
| | | | | | insert |
| TI-1539 | GTTC | FnCas12a | 12407:12429 | + | Transgenic |
| | | | | | insert |
| TI-1540 | TTCC | LbCas12a-TYC | 12408:12430 | + | Transgenic |
| | | | | | insert |
| TI-1541 | TCCA | LbCas12a-TYC | 12409:12431 | + | Transgenic |
| | | | | | insert |
| TI-1542 | CTTG | FnCas12a | 12398:12420 | − | Transgenic |
| | | | | | insert |
| TI-1543 | TTCA | LbCas12a-TYC | 12402:12424 | − | Transgenic |
| | | | | | insert |
| TI-1544 | CTTC | FnCas12a | 12403:12425 | − | Transgenic |
| | | | | | insert |
| TI-1545 | TCCG | LbCas12a-TYC | 12434:12456 | + | Transgenic |
| | | | | | insert |
| TI-1546 | GTTC | FnCas12a | 12440:12462 | + | Transgenic |
| | | | | | insert |
| TI-1547 | TCCA | LbCas12a-TYC | 12424:12446 | − | Transgenic |
| | | | | | insert |
| TI-1548 | TTCA | LbCas12a-TYC | 12429:12451 | − | Transgenic |
| | | | | | insert |
| TI-1549 | GTTC | FnCas12a | 12430:12452 | − | Transgenic |
| | | | | | insert |
| TI-1550 | CTTC | FnCas12a | 12487:12509 | − | Transgenic |
| | | | | | insert |
| TI-1551 | CTTC | FnCas12a | 12521:12543 | + | Transgenic |
| | | | | | insert |
| TI-1552 | TTCA | LbCas12a-TYC | 12522:12544 | + | Transgenic |
| | | | | | insert |
| TI-1553 | TTTC | LbCas12a | 12529:12551 | + | Transgenic |
| | | | | | insert |
| TI-1554 | TTCG | LbCas12a-TYC | 12530:12552 | + | Transgenic |
| | | | | | insert |
| TI-1555 | GTTA | FnCas12a | 12511:12533 | − | Transgenic |
| | | | | | insert |
| TI-1556 | TTCA | LbCas12a-TYC | 12523:12545 | − | Transgenic |
| | | | | | insert |
| TI-1557 | TTTC | LbCas12a | 12524:12546 | − | Transgenic |
| | | | | | insert |
| TI-1558 | GTTG | FnCas12a | 12548:12570 | + | Transgenic |
| | | | | | insert |
| TI-1559 | TCCG | LbCas12a-TYC | 12534:12556 | − | Transgenic |
| | | | | | insert |
| TI-1560 | TTCG | LbCas12a-TYC | 12579:12601 | + | Transgenic |
| | | | | | insert |
| TI-1561 | TCCA | LbCas12a-TYC | 12558:12580 | − | Transgenic |
| | | | | | insert |
| TI-1562 | TCCC | LbCas12a-TYC | 12562:12584 | − | Transgenic |
| | | | | | insert |
| TI-1563 | TTCC | LbCas12a-TYC | 12563:12585 | − | Transgenic |
| | | | | | insert |
| TI-1564 | CTTC | FnCas12a | 12564:12586 | − | Transgenic |
| | | | | | insert |
| TI-1565 | TCCC | LbCas12a-TYC | 12568:12590 | − | Transgenic |
| | | | | | insert |
| TI-1566 | TTCC | LbCas12a-TYC | 12569:12591 | − | Transgenic |
| | | | | | insert |
| TI-1567 | TTTC | LbCas12a | 12570:12592 | − | Transgenic |
| | | | | | insert |
| TI-1568 | CTTG | FnCas12a | 12584:12606 | − | Transgenic |
| | | | | | insert |
| TI-1569 | TCCC | LbCas12a-TYC | 12634:12656 | + | Transgenic |
| | | | | | insert |
| TI-1570 | TCCA | LbCas12a-TYC | 12638:12660 | + | Transgenic |
| | | | | | insert |
| TI-1571 | GTTC | FnCas12a | 12649:12671 | + | Transgenic |
| | | | | | insert |
| TI-1572 | TTCC | LbCas12a-TYC | 12650:12672 | + | Transgenic |
| | | | | | insert |
| TI-1573 | TCCC | LbCas12a-TYC | 12651:12673 | + | Transgenic |
| | | | | | insert |
| TI-1574 | GTTG | FnCas12a | 12659:12681 | + | Transgenic |
| | | | | | insert |
| TI-1575 | CTTC | FnCas12a | 12668:12690 | + | Transgenic |
| | | | | | insert |
| TI-1576 | TCCG | LbCas12a-TYC | 12683:12705 | + | Transgenic |
| | | | | | insert |
| TI-1577 | TTTA | LbCas12a | 12672:12694 | − | Transgenic |
| | | | | | insert |
| TI-1578 | TTCA | LbCas12a-TYC | 12684:12706 | − | Transgenic |
| | | | | | insert |
| TI-1579 | GTTC | FnCas12a | 12696:12718 | − | Transgenic |
| | | | | | insert |
| TI-1580 | TCCG | LbCas12a-TYC | 12700:12722 | − | Transgenic |
| | | | | | insert |
| TI-1581 | TATC | LbCas12a-TATV | 12706:12728 | − | Transgenic |
| | | | | | insert |
| TI-1582 | CTTA | FnCas12a | 12737:12759 | + | Transgenic |
| | | | | | insert |
| TI-1583 | TTCC | LbCas12a-TYC | 12721:12743 | − | Transgenic |
| | | | | | insert |
| TI-1584 | TTTC | LbCas12a | 12722:12744 | − | Transgenic |
| | | | | | insert |
| TI-1585 | TATC | LbCas12a-TATV | 12734:12756 | − | Transgenic |
| | | | | | insert |
| TI-1586 | TTCA | LbCas12a-TYC | 12739:12761 | − | Transgenic |
| | | | | | insert |
| TI-1587 | CTTC | FnCas12a | 12740:12762 | − | Transgenic |
| | | | | | insert |
| TI-1588 | GTTA | FnCas12a | 12770:12792 | + | Transgenic |
| | | | | | insert |
| TI-1589 | TATC | LbCas12a-TATV | 12772:12794 | + | Transgenic |
| | | | | | insert |
| TI-1590 | TCCG | LbCas12a-TYC | 12763:12785 | − | Transgenic |
| | | | | | insert |
| TI-1591 | TTTG | LbCas12a | 12811:12833 | + | Transgenic |
| | | | | | insert |
| TI-1592 | CTTC | FnCas12a | 12825:12847 | + | Transgenic |
| | | | | | insert |
| TI-1593 | TTCA | LbCas12a-TYC | 12807:12829 | − | Transgenic |
| | | | | | insert |
| TI-1594 | TTTC | LbCas12a | 12808:12830 | − | Transgenic |
| | | | | | insert |
| TI-1595 | GTTA | FnCas12a | 12842:12864 | + | Transgenic |
| | | | | | insert |
| TI-1596 | TTCA | LbCas12a-TYC | 12826:12848 | − | Transgenic |
| | | | | | insert |
| TI-1597 | CTTC | FnCas12a | 12827:12849 | − | Transgenic |
| | | | | | insert |
| TI-1598 | TATC | LbCas12a-TATV | 12830:12852 | − | Transgenic |
| | | | | | insert |
| TI-1599 | CTTC | FnCas12a | 12867:12889 | + | Transgenic |
| | | | | | insert |
| TI-1600 | TTCA | LbCas12a-TYC | 12868:12890 | + | Transgenic |
| | | | | | insert |
| TI-1601 | TATC | LbCas12a-TATV | 12884:12906 | + | Transgenic |
| | | | | | insert |
| TI-1602 | TCCA | LbCas12a-TYC | 12886:12908 | + | Transgenic |
| | | | | | insert |
| TI-1603 | CTTG | FnCas12a | 12893:12915 | + | Transgenic |
| | | | | | insert |
| TI-1604 | TTTG | LbCas12a | 12911:12933 | + | Transgenic |
| | | | | | insert |
| TI-1605 | TTCA | LbCas12a-TYC | 12906:12928 | − | Transgenic |
| | | | | | insert |
| TI-1606 | TCCA | LbCas12a-TYC | 12910:12932 | − | Transgenic |
| | | | | | insert |
| TI-1607 | GTTG | FnCas12a | 12949:12971 | + | Transgenic |
| | | | | | insert |
| TI-1608 | TTCA | LbCas12a-TYC | 12930:12952 | − | Transgenic |
| | | | | | insert |
| TI-1609 | CTTC | FnCas12a | 12931:12953 | − | Transgenic |
| | | | | | insert |
| TI-1610 | TTCC | LbCas12a-TYC | 12934:12956 | − | Transgenic |
| | | | | | insert |
| TI-1611 | TCCG | LbCas12a-TYC | 12965:12987 | + | Transgenic |
| | | | | | insert |
| TI-1612 | GTTA | FnCas12a | 12961:12983 | − | Transgenic |
| | | | | | insert |
| TI-1613 | TCCG | LbCas12a-TYC | 12964:12986 | − | Transgenic |
| | | | | | insert |
| TI-1614 | TTTA | LbCas12a | 12970:12992 | − | Transgenic |
| | | | | | insert |
| TI-1615 | CTTA | FnCas12a | 12995:13017 | + | Transgenic |
| | | | | | insert |
| TI-1616 | GTTG | FnCas12a | 13010:13032 | + | Transgenic |
| | | | | | insert |
| TI-1617 | CTTG | FnCas12a | 13036:13058 | + | Transgenic |
| | | | | | insert |
| TI-1618 | TCCG | LbCas12a-TYC | 13044:13066 | + | Transgenic |
| | | | | | insert |
| TI-1619 | TCCG | LbCas12a-TYC | 13033:13055 | − | Transgenic |
| | | | | | insert |
| TI-1620 | TTCC | LbCas12a-TYC | 13034:13056 | − | Transgenic |
| | | | | | insert |
| TI-1621 | TTTC | LbCas12a | 13035:13057 | − | Transgenic |
| | | | | | insert |
| TI-1622 | TTTG | LbCas12a | 13069:13091 | + | Transgenic |
| | | | | | insert |
| TI-1623 | GTTG | FnCas12a | 13048:13070 | − | Transgenic |
| | | | | | insert |
| TI-1624 | CTTC | FnCas12a | 13080:13102 | + | Transgenic |
| | | | | | insert |
| TI-1625 | TTCG | LbCas12a-TYC | 13081:13103 | + | Transgenic |
| | | | | | insert |
| TI-1626 | GTTG | FnCas12a | 13071:13093 | − | Transgenic |
| | | | | | insert |
| TI-1627 | GTTG | FnCas12a | 13094:13116 | + | Transgenic |
| | | | | | insert |
| TI-1628 | TCCA | LbCas12a-TYC | 13080:13102 | − | Transgenic |
| | | | | | insert |
| TI-1629 | CTTG | FnCas12a | 13105:13127 | + | Transgenic |
| | | | | | insert |
| TI-1630 | TCCG | LbCas12a-TYC | 13088:13110 | − | Transgenic |
| | | | | | insert |
| TI-1631 | TATC | LbCas12a-TATV | 13090:13112 | − | Transgenic |
| | | | | | insert |
| TI-1632 | TTTC | LbCas12a | 13130:13152 | + | Transgenic |
| | | | | | insert |
| TI-1633 | CTTA | FnCas12a | 13122:13144 | − | Transgenic |
| | | | | | insert |
| TI-1634 | GTTG | FnCas12a | 13152:13174 | − | Transgenic |
| | | | | | insert |
| TI-1635 | TATG | LbCas12a-TATV | 13183:13205 | + | Transgenic |
| | | | | | insert |
| TI-1636 | CTTC | FnCas12a | 13194:13216 | + | Transgenic |
| | | | | | insert |
| TI-1637 | TTCC | LbCas12a-TYC | 13195:13217 | + | Transgenic |
| | | | | | insert |
| TI-1638 | CTTC | FnCas12a | 13198:13220 | + | Transgenic |
| | | | | | insert |
| TI-1639 | TTCC | LbCas12a-TYC | 13199:13221 | + | Transgenic |
| | | | | | insert |
| TI-1640 | TCCC | LbCas12a-TYC | 13200:13222 | + | Transgenic |
| | | | | | insert |
| TI-1641 | TCCA | LbCas12a-TYC | 13185:13207 | − | Transgenic |
| | | | | | insert |
| TI-1642 | TTTA | LbCas12a | 13208:13230 | + | Transgenic |
| | | | | | insert |
| TI-1643 | TATG | LbCas12a-TATV | 13210:13232 | + | Transgenic |
| | | | | | insert |
| TI-1644 | TTTA | LbCas12a | 13216:13238 | + | Transgenic |
| | | | | | insert |
| TI-1645 | TTTG | LbCas12a | 13209:13231 | − | Transgenic |
| | | | | | insert |
| TI-1646 | TTCA | LbCas12a-TYC | 13216:13238 | − | Transgenic |
| | | | | | insert |
| TI-1647 | GTTC | FnCas12a | 13217:13239 | − | Transgenic |
| | | | | | insert |
| TI-1648 | TATC | LbCas12a-TATV | 13226:13248 | − | Transgenic |
| | | | | | insert |
| TI-1649 | TTTG | LbCas12a | 13231:13253 | − | Transgenic |
| | | | | | insert |
| TI-1650 | GTTA | FnCas12a | 13241:13263 | − | Transgenic |
| | | | | | insert |
| TI-1651 | TCCG | LbCas12a-TYC | 13245:13267 | − | Transgenic |
| | | | | | insert |
| TI-1652 | TTTC | LbCas12a | 13291:13313 | + | Transgenic |
| | | | | | insert |
| TI-1653 | TTCG | LbCas12a-TYC | 13292:13314 | + | Transgenic |
| | | | | | insert |
| TI-1654 | GTTC | FnCas12a | 13295:13317 | + | Transgenic |
| | | | | | insert |
| TI-1655 | TTCG | LbCas12a-TYC | 13296:13318 | + | Transgenic |
| | | | | | insert |
| TI-1656 | TATC | LbCas12a-TATV | 13300:13322 | + | Transgenic |
| | | | | | insert |
| TI-1657 | TTTC | LbCas12a | 13309:13331 | + | Transgenic |
| | | | | | insert |
| TI-1658 | GTTG | FnCas12a | 13288:13310 | − | Transgenic |
| | | | | | insert |
| TI-1659 | TTCG | LbCas12a-TYC | 13310:13332 | + | Transgenic |
| | | | | | insert |
| TI-1660 | GTTC | FnCas12a | 13319:13341 | + | Transgenic |
| | | | | | insert |
| TI-1661 | CTTG | FnCas12a | 13298:13320 | − | Transgenic |
| | | | | | insert |
| TI-1662 | TTCG | LbCas12a-TYC | 13320:13342 | + | Transgenic |
| | | | | | insert |
| TI-1663 | GTTC | FnCas12a | 13328:13350 | + | Transgenic |
| | | | | | insert |
| TI-1664 | TTCA | LbCas12a-TYC | 13329:13351 | + | Transgenic |
| | | | | | insert |
| TI-1665 | TTTC | LbCas12a | 13342:13364 | + | Transgenic |
| | | | | | insert |
| TI-1666 | TTCA | LbCas12a-TYC | 13343:13365 | + | Transgenic |
| | | | | | insert |
| TI-1667 | TTTG | LbCas12a | 13374:13396 | + | Transgenic |
| | | | | | insert |
| TI-1668 | TCCC | LbCas12a-TYC | 13365:13387 | − | Transgenic |
| | | | | | insert |
| TI-1669 | TTCC | LbCas12a-TYC | 13366:13388 | − | Transgenic |
| | | | | | insert |
| TI-1670 | TTTC | LbCas12a | 13367:13389 | − | Transgenic |
| | | | | | insert |
| TI-1671 | TTTC | LbCas12a | 13404:13426 | + | Transgenic |
| | | | | | insert |
| TI-1672 | CTTG | FnCas12a | 13407:13429 | + | Transgenic |
| | | | | | insert |
| TI-1673 | TTTG | LbCas12a | 13416:13438 | + | Transgenic |
| | | | | | insert |
| TI-1674 | TTTA | LbCas12a | 13444:13466 | + | Transgenic |
| | | | | | insert |
| TI-1675 | TTCG | LbCas12a-TYC | 13448:13470 | + | Transgenic |
| | | | | | insert |
| TI-1676 | TTTC | LbCas12a | 13454:13476 | + | Transgenic |
| | | | | | insert |
| TI-1677 | TTCG | LbCas12a-TYC | 13455:13477 | + | Transgenic |
| | | | | | insert |
| TI-1678 | TATC | LbCas12a-TATV | 13460:13482 | + | Transgenic |
| | | | | | insert |
| TI-1679 | TTCA | LbCas12a-TYC | 13443:13465 | − | Transgenic |
| | | | | | insert |
| TI-1680 | TTTC | LbCas12a | 13444:13466 | − | Transgenic |
| | | | | | insert |
| TI-1681 | TCCA | LbCas12a-TYC | 13448:13470 | − | Transgenic |
| | | | | | insert |
| TI-1682 | TTCC | LbCas12a-TYC | 13449:13471 | − | Transgenic |
| | | | | | insert |
| TI-1683 | TTTC | LbCas12a | 13450:13472 | − | Transgenic |
| | | | | | insert |
| TI-1684 | TCCA | LbCas12a-TYC | 13454:13476 | − | Transgenic |
| | | | | | insert |
| TI-1685 | TCCA | LbCas12a-TYC | 13458:13480 | − | Transgenic |
| | | | | | insert |
| TI-1686 | CTTC | FnCas12a | 13462:13484 | − | Transgenic |
| | | | | | insert |
| TI-1687 | GTTA | FnCas12a | 13494:13516 | + | Transgenic |
| | | | | | insert |
| TI-1688 | TTCA | LbCas12a-TYC | 13472:13494 | − | Transgenic |
| | | | | | insert |
| TI-1689 | TATC | LbCas12a-TATV | 13477:13499 | − | Transgenic |
| | | | | | insert |
| TI-1690 | TATG | LbCas12a-TATV | 13506:13528 | + | Transgenic |
| | | | | | insert |
| TI-1691 | TTTG | LbCas12a | 13500:13522 | − | Transgenic |
| | | | | | insert |
| TI-1692 | TTTC | LbCas12a-TYC | 13547:13569 | + | Transgenic |
| | | | | | insert |
| TI-1693 | GTTG | FnCas12a | 13559:13581 | + | Transgenic |
| | | | | | insert |
| TI-1694 | TTCA | LbCas12a-TYC | 13547:13569 | − | Transgenic |
| | | | | | insert |
| TI-1695 | TTTC | LbCas12a | 13548:13570 | − | Transgenic |
| | | | | | insert |
| TI-1696 | TTTG | LbCas12a | 13572:13594 | + | Transgenic |
| | | | | | insert |
| TI-1697 | TATA | LbCas12a-TATV | 13553:13575 | − | Transgenic |
| | | | | | insert |
| TI-1698 | CTTA | FnCas12a | 13555:13577 | − | Transgenic |
| | | | | | insert |
| TI-1699 | TATA | LbCas12a-TATV | 13580:13602 | + | Transgenic |
| | | | | | insert |
| TI-1700 | TATC | LbCas12a-TATV | 13560:13582 | − | Transgenic |
| | | | | | insert |
| TI-1701 | TTTG | LbCas12a | 13566:13588 | − | Transgenic |
| | | | | | insert |
| TI-1702 | TATG | LbCas12a-TATV | 13589:13611 | + | Transgenic |
| | | | | | insert |
| TI-1703 | TTTA | LbCas12a | 13584:13606 | − | Transgenic |
| | | | | | insert |
| TI-1704 | TTCG | LbCas12a-TYC | 13616:13638 | − | Transgenic |
| | | | | | insert |
| TI-1705 | CTTC | FnCas12a | 13617:13639 | − | Transgenic |
| | | | | | insert |
| TI-1706 | GTTA | FnCas12a | 13647:13669 | + | Transgenic |
| | | | | | insert |
| TI-1707 | TATG | LbCas12a-TATV | 13652:13674 | + | Transgenic |
| | | | | | insert |
| TI-1708 | TTTA | LbCas12a | 13634:13656 | − | Transgenic |
| | | | | | insert |
| TI-1709 | CTTG | FnCas12a | 13668:13690 | + | Transgenic |
| | | | | | insert |
| TI-1710 | GTTG | FnCas12a | 13674:13696 | + | Transgenic |
| | | | | | insert |
| TI-1711 | TATG | LbCas12a-TATV | 13684:13706 | + | Transgenic |
| | | | | | insert |
| TI-1712 | CTTA | FnCas12a | 13688:13710 | + | Transgenic |
| | | | | | insert |
| TI-1713 | TTCA | LbCas12a-TYC | 13692:13714 | + | Transgenic |
| | | | | | insert |
| TI-1714 | GTTG | FnCas12a | 13674:13696 | − | Transgenic |
| | | | | | insert |
| TI-1715 | TTTG | LbCas12a | 13677:13699 | − | Transgenic |
| | | | | | insert |
| TI-1716 | TATA | LbCas12a-TATV | 13681:13703 | − | Transgenic |
| | | | | | insert |
| TI-1717 | TATA | LbCas12a-TATV | 13708:13730 | + | Transgenic |
| | | | | | insert |
| TI-1718 | TTTC | LbCas12a | 13713:13735 | + | Transgenic |
| | | | | | insert |
| TI-1719 | TTCA | LbCas12a-TYC | 13714:13736 | + | Transgenic |
| | | | | | insert |
| TI-1720 | TTTC | LbCas12a | 13697:13719 | − | Transgenic |
| | | | | | insert |
| TI-1721 | TTTG | LbCas12a | 13706:13728 | − | Transgenic |
| | | | | | insert |
| TI-1722 | GTTA | FnCas12a | 13738:13760 | + | Transgenic |
| | | | | | insert |
| TI-1723 | TTCA | LbCas12a-TYC | 13716:13738 | − | Transgenic |
| | | | | | insert |
| TI-1724 | CTTG | FnCas12a | 13722:13744 | − | Transgenic |
| | | | | | insert |
| TI-1725 | TATG | LbCas12a-TATV | 13753:13775 | + | Transgenic |
| | | | | | insert |
| TI-1726 | TTTA | LbCas12a | 13768:13790 | + | Transgenic |
| | | | | | insert |
| TI-1727 | TTCA | LbCas12a-TYC | 13753:13775 | − | Transgenic |
| | | | | | insert |
| TI-1728 | GTTC | FnCas12a | 13754:13776 | − | Transgenic |
| | | | | | insert |
| TI-1729 | TTTA | LbCas12a-TATV | 13776:13798 | + | Transgenic |
| | | | | | insert |
| TI-1730 | TATG | LbCas12a-TATV | 13778:13800 | + | Transgenic |
| | | | | | insert |
| TI-1731 | TTTC | LbCas12a | 13785:13807 | + | Transgenic |
| | | | | | insert |
| TI-1732 | TTCC | LbCas12a-TYC | 13786:13808 | + | Transgenic |
| | | | | | insert |
| TI-1733 | TTTA | LbCas12a | 13790:13812 | + | Transgenic |
| | | | | | insert |
| TI-1734 | TATG | LbCas12a-TATV | 13792:13814 | + | Transgenic |
| | | | | | insert |
| TI-1735 | TTTC | LbCas12a | 13800:13822 | + | Transgenic |
| | | | | | insert |
| TI-1736 | TTCC | LbCas12a-TYC | 13801:13823 | + | Transgenic |
| | | | | | insert |
| TI-1737 | TCCA | LbCas12a-TYC | 13802:13824 | + | Transgenic |
| | | | | | insert |
| TI-1738 | CTTG | FnCas12a | 13811:13833 | + | Transgenic |
| | | | | | insert |
| TI-1739 | TATA | LbCas12a-TATV | 13808:13830 | − | Transgenic |
| | | | | | insert |
| TI-1740 | TTTA | LbCas12a | 13833:13855 | + | Transgenic |
| | | | | | insert |
| TI-1741 | TATA | LbCas12a-TATV | 13835:13857 | + | Transgenic |
| | | | | | insert |
| TI-1742 | TATA | LbCas12a-TATV | 13814:13836 | − | Transgenic |
| | | | | | insert |
| TI-1743 | TATA | LbCas12a-TATV | 13841:13863 | + | Transgenic |
| | | | | | insert |
| TI-1744 | GTTA | FnCas12a | 13845:13867 | + | Transgenic |
| | | | | | insert |
| TI-1745 | TATA | LbCas12a-TATV | 13847:13869 | + | Transgenic |
| | | | | | insert |
| TI-1746 | TCCA | LbCas12a-TYC | 13828:13850 | − | Transgenic |
| | | | | | insert |
| TI-1747 | TTCA | LbCas12a-TYC | 13846:13868 | − | Transgenic |
| | | | | | insert |
| TI-1748 | TTTC | LbCas12a | 13847:13869 | − | Transgenic |
| | | | | | insert |
| TI-1749 | TTTA | LbCas12a | 13884:13906 | + | Transgenic |
| | | | | | insert |
| TI-1750 | TTTA | LbCas12a | 13894:13916 | + | Transgenic |
| | | | | | insert |
| TI-1751 | TATG | LbCas12a-TATV | 13896:13918 | + | Transgenic |
| | | | | | insert |
| TI-1752 | CTTG | FnCas12a | 13901:13923 | + | Transgenic |
| | | | | | insert |
| TI-1753 | GTTG | FnCas12a | 13890:13912 | − | Transgenic |
| | | | | | insert |
| TI-1754 | GTTA | FnCas12a | 13913:13935 | − | Transgenic |
| | | | | | insert |
| TI-1755 | CTTA | FnCas12a | 13938:13960 | + | Transgenic |
| | | | | | insert |
| TI-1756 | GTTA | FnCas12a | 13917:13939 | − | Transgenic |
| | | | | | insert |
| TI-1757 | CTTA | FnCas12a | 13955:13977 | + | Transgenic |
| | | | | | insert |
| TI-1758 | TCCG | LbCas12a-TYC | 13937:13959 | − | Transgenic |
| | | | | | insert |
| TI-1759 | TCCG | LbCas12a-TYC | 13950:13972 | − | Transgenic |
| | | | | | insert |
| TI-1760 | CTTG | FnCas12a | 13958:13980 | − | Transgenic |
| | | | | | insert |
| TI-1761 | GTTC | FnCas12a | 13982:14004 | + | Transgenic |
| | | | | | insert |
| TI-1762 | TTCA | LbCas12a-TYC | 13983:14005 | + | Transgenic |
| | | | | | insert |
| TI-1763 | CTTC | FnCas12a | 14031:14053 | + | Transgenic |
| | | | | | insert |
| TI-1764 | TTCC | LbCas12a-TYC | 14032:14054 | + | Transgenic |
| | | | | | insert |
| TI-1765 | TCCA | LbCas12a-TYC | 14033:14055 | + | Transgenic |
| | | | | | insert |
| TI-1766 | CTTA | FnCas12a | 14038:14060 | + | Transgenic |
| | | | | | insert |
| TI-1767 | TATC | LbCas12a-TATV | 14040:14062 | + | Transgenic |
| | | | | | insert |
| TI-1768 | GTTA | FnCas12a | 14028:14050 | − | Transgenic |
| | | | | | insert |
| TI-1769 | TTCA | LbCas12a-TYC | 14058:14080 | − | Transgenic |
| | | | | | insert |
| TI-1770 | TTTC | LbCas12a | 14059:14081 | − | Transgenic |
| | | | | | insert |
| TI-1771 | CTTA | FnCas12a | 14063:14085 | − | Transgenic |
| | | | | | insert |
| TI-1772 | GTTA | FnCas12a | 14074:14096 | − | Transgenic |
| | | | | | insert |
| TI-1773 | GTTG | FnCas12a | 14079:14101 | − | Transgenic |
| | | | | | insert |
| TI-1774 | TTCG | LbCas12a-TYC | 14090:14112 | − | Transgenic |
| | | | | | insert |
| TI-1775 | GTTC | FnCas12a | 14091:14113 | − | Transgenic |
| | | | | | insert |
| TI-1776 | GTTA | FnCas12a | 14125:14147 | + | Transgenic |
| | | | | | insert |
| TI-1777 | TCCC | LbCas12a-TYC | 14104:14126 | − | Transgenic |
| | | | | | insert |
| TI-1778 | TATC | LbCas12a-TATV | 14106:14128 | − | Transgenic |
| | | | | | insert |
| TI-1779 | TTTC | LbCas12a | 14140:14162 | + | Transgenic |
| | | | | | insert |
| TI-1780 | TTCG | LbCas12a-TYC | 14141:14163 | + | Transgenic |
| | | | | | insert |
| TI-1781 | TCCC | LbCas12a-TYC | 14126:14148 | − | Transgenic |
| | | | | | insert |
| TI-1782 | TATA | LbCas12a-TATV | 14139:14161 | − | Transgenic |
| | | | | | insert |
| TI-1783 | CTTA | FnCas12a | 14164:14186 | + | Transgenic |
| | | | | | insert |
| TI-1784 | TATA | LbCas12a-TATV | 14166:14188 | + | Transgenic |
| | | | | | insert |
| TI-1785 | TCCC | LbCas12a-TYC | 14199:14221 | + | Transgenic |
| | | | | | insert |
| TI-1786 | TTTG | LbCas12a | 14207:14229 | + | Transgenic |
| | | | | | insert |
| TI-1787 | TCCC | LbCas12a-TYC | 14193:14215 | − | Transgenic |
| | | | | | insert |
| TI-1788 | TTCC | LbCas12a-TYC | 14194:14216 | − | Transgenic |
| | | | | | insert |
| TI-1789 | TTTC | LbCas12a | 14195:14217 | − | Transgenic |
| | | | | | insert |
| TI-1790 | TATC | LbCas12a-TATV | 14217:14239 | − | Transgenic |
| | | | | | insert |
| TI-1791 | GTTC | FnCas12a | 14259:14281 | + | Transgenic |
| | | | | | insert |
| TI-1792 | CTTG | FnCas12a | 14268:14290 | + | Transgenic |
| | | | | | insert |
| TI-1793 | GTTA | FnCas12a | 14271:14293 | + | Transgenic |
| | | | | | insert |
| TI-1794 | TTTA | LbCas12a | 14258:14280 | − | Transgenic |
| | | | | | insert |
| TI-1795 | GTTA | FnCas12a | 14263:14285 | − | Transgenic |
| | | | | | insert |
| TI-1796 | GTTC | FnCas12a | 14271:14293 | − | Transgenic |
| | | | | | insert |
| TI-1797 | CTTG | FnCas12a | 14303:14325 | + | Transgenic |
| | | | | | insert |
| TI-1798 | GTTG | FnCas12a | 14291:14313 | − | Transgenic |
| | | | | | insert |
| TI-1799 | TTTA | LbCas12a | 14299:14321 | − | Transgenic |
| | | | | | insert |
| TI-1800 | TCCG | LbCas12a-TYC | 14307:14329 | − | Transgenic |
| | | | | | insert |
| TI-1801 | CTTA | FnCas12a | 14363:14385 | + | Transgenic |
| | | | | | insert |
| TI-1802 | CTTC | FnCas12a | 14380:14402 | + | Transgenic |
| | | | | | insert |
| TI-1803 | TTCC | LbCas12a-TYC | 14381:14403 | + | Transgenic |
| | | | | | insert |
| TI-1804 | TCCC | LbCas12a-TYC | 14382:14404 | + | Transgenic |
| | | | | | insert |
| TI-1805 | GTTG | FnCas12a | 14363:14385 | − | Transgenic |
| | | | | | insert |
| TI-1806 | CTTA | FnCas12a | 14395:14417 | + | Transgenic |
| | | | | | insert |
| TI-1807 | CTTC | FnCas12a | 14411:14433 | + | Transgenic |
| | | | | | insert |
| TI-1808 | TTCA | LbCas12a-TYC | 14412:14434 | + | Transgenic |
| | | | | | insert |
| TI-1809 | TATA | LbCas12a-TATV | 14390:14412 | − | Transgenic |
| | | | | | insert |
| TI-1810 | TATA | LbCas12a-TATV | 14417:14439 | + | Transgenic |
| | | | | | insert |
| TI-1811 | GTTG | FnCas12a | 14449:14471 | + | Transgenic |
| | | | | | insert |
| TI-1812 | TTTG | LbCas12a | 14442:14464 | − | Transgenic |
| | | | | | insert |
| TI-1813 | CTTA | FnCas12a | 14465:14487 | + | Transgenic |
| | | | | | insert |
| TI-1814 | TTTA | LbCas12a | 14446:14468 | − | Transgenic |
| | | | | | insert |
| TI-1815 | TCCG | LbCas12a-TYC | 14499:14521 | + | Transgenic |
| | | | | | insert |
| TI-1816 | CTTG | FnCas12a | 14509:14531 | + | Transgenic |
| | | | | | insert |
| TI-1817 | GTTG | FnCas12a | 14494:14516 | − | Transgenic |
| | | | | | insert |
| TI-1818 | TTCG | LbCas12a-TYC | 14502:14524 | − | Transgenic |
| | | | | | insert |
| TI-1819 | CTTC | FnCas12a | 14503:14525 | − | Transgenic |
| | | | | | insert |
| TI-1820 | TCCG | LbCas12a-TYC | 14525:14547 | + | Transgenic |
| | | | | | insert |
| TI-1821 | TCCG | LbCas12a-TYC | 14513:14535 | − | Transgenic |
| | | | | | insert |
| TI-1822 | TCCG | LbCas12a-TYC | 14519:14541 | − | Transgenic |
| | | | | | insert |
| TI-1823 | TTCC | LbCas12a-TYC | 14520:14542 | − | Transgenic |
| | | | | | insert |
| TI-1824 | TTTC | LbCas12a | 14521:14543 | − | Transgenic |
| | | | | | insert |
| TI-1825 | GTTA | FnCas12a | 14560:14582 | + | Transgenic |
| | | | | | insert |
| TI-1826 | TCCG | LbCas12a-TYC | 14584:14606 | + | Transgenic |
| | | | | | insert |
| TI-1827 | TTCC | LbCas12a-TYC | 14572:14594 | − | Transgenic |
| | | | | | insert |
| TI-1828 | GTTA | FnCas12a | 14595:14617 | + | Transgenic |
| | | | | | insert |
| TI-1829 | TCCA | LbCas12a-TYC | 14603:14625 | + | Transgenic |
| | | | | | insert |
| TI-1830 | TATC | LbCas12a-TATV | 14581:14603 | − | Transgenic |
| | | | | | insert |
| TI-1831 | GTTG | FnCas12a | 14621:14643 | + | Transgenic |
| | | | | | insert |
| TI-1832 | TCCC | LbCas12a-TYC | 14634:14656 | + | Transgenic |
| | | | | | insert |
| TI-1833 | TCCA | LbCas12a-TYC | 14617:14639 | − | Transgenic |
| | | | | | insert |
| TI-1834 | TATC | LbCas12a-TATV | 14639:14661 | + | Transgenic |
| | | | | | insert |
| TI-1835 | TTTC | LbCas12a | 14649:14671 | + | Transgenic |
| | | | | | insert |
| TI-1836 | TTCG | LbCas12a-TYC | 14650:14672 | + | Transgenic |
| | | | | | insert |
| TI-1837 | TTTG | LbCas12a | 14655:14677 | + | Transgenic |
| | | | | | insert |
| TI-1838 | TCCG | LbCas12a-TYC | 14655:14677 | − | Transgenic |
| | | | | | insert |
| TI-1839 | TCCG | LbCas12a-TYC | 14709:14731 | + | Transgenic |
| | | | | | insert |
| TI-1840 | TTTG | LbCas12a | 14702:14724 | − | Transgenic |
| | | | | | insert |
| TI-1841 | TTTC | LbCas12a | 14726:14748 | + | Transgenic |
| | | | | | insert |
| TI-1842 | TTCA | LbCas12a-TYC | 14727:14749 | + | Transgenic |
| | | | | | insert |
| TI-1843 | TTTC | LbCas12a | 14736:14758 | + | Transgenic |
| | | | | | insert |
| TI-1844 | TTCA | LbCas12a-TYC | 14737:14759 | + | Transgenic |
| | | | | | insert |
| TI-1845 | CTTG | FnCas12a | 14741:14763 | + | Transgenic |
| | | | | | insert |
| TI-1846 | GTTC | FnCas12a | 14748:14770 | + | Transgenic |
| | | | | | insert |
| TI-1847 | TTCC | LbCas12a-TYC | 14749:14771 | + | Transgenic |
| | | | | | insert |
| TI-1848 | CTTC | FnCas12a | 14752:14774 | + | Transgenic |
| | | | | | insert |
| TI-1849 | GTTC | FnCas12a | 14757:14779 | + | Transgenic |
| | | | | | insert |
| TI-1850 | TTCA | LbCas12a-TYC | 14758:14780 | + | Transgenic |
| | | | | | insert |
| TI-1851 | CTTG | FnCas12a | 14753:14775 | − | Transgenic |
| | | | | | insert |
| TI-1852 | GTTG | FnCas12a | 14762:14784 | − | Transgenic |
| | | | | | insert |
| TI-1853 | CTTC | FnCas12a | 14804:14826 | + | Transgenic |
| | | | | | insert |
| TI-1854 | CTTG | FnCas12a | 14807:14829 | + | Transgenic |
| | | | | | insert |
| TI-1855 | GTTC | FnCas12a | 14815:14837 | + | Transgenic |
| | | | | | insert |
| TI-1856 | TTCA | LbCas12a-TYC | 14816:14838 | + | Transgenic |
| | | | | | insert |
| TI-1857 | TCCG | LbCas12a-TYC | 14832:14854 | − | Transgenic |
| | | | | | insert |
| TI-1858 | TCCC | LbCas12a-TYC | 14842:14864 | − | Transgenic |
| | | | | | insert |
| TI-1859 | GTTG | FnCas12a | 14871:14893 | − | Transgenic |
| | | | | | insert |
| TI-1860 | TTTG | LbCas12a | 14874:14896 | − | Transgenic |
| | | | | | insert |
| TI-1861 | TCCC | LbCas12a-TYC | 14879:14901 | − | Transgenic |
| | | | | | insert |
| TI-1862 | TTCC | LbCas12a-TYC | 14880:14902 | − | Transgenic |
| | | | | | insert |
| TI-1863 | TTTA | LbCas12a | 14914:14936 | + | Transgenic |
| | | | | | insert |
| TI-1864 | TTCG | LbCas12a-TYC | 14905:14927 | − | Transgenic |
| | | | | | insert |
| TI-1865 | TTTC | LbCas12a | 14906:14928 | − | Transgenic |
| | | | | | insert |
| TI-1866 | TCCC | LbCas12a-TYC | 14929:14951 | + | Transgenic |
| | | | | | insert |
| TI-1867 | TCCC | LbCas12a-TYC | 14914:14936 | − | Transgenic |
| | | | | | insert |
| TI-1868 | TTCC | LbCas12a-TYC | 14918:14940 | − | Transgenic |
| | | | | | insert |
| TI-1869 | CTTC | FnCas12a | 14919:14941 | − | Transgenic |
| | | | | | insert |
| TI-1870 | CTTA | FnCas12a | 14950:14972 | + | Transgenic |
| | | | | | insert |
| TI-1871 | GTTA | FnCas12a | 14954:14976 | + | Transgenic |
| | | | | | insert |
| TI-1872 | TTTA | LbCas12a | 14939:14961 | − | Transgenic |
| | | | | | insert |
| TI-1873 | TTTA | LbCas12a | 14964:14986 | + | Transgenic |
| | | | | | insert |
| TI-1874 | TATA | LbCas12a-TATV | 14948:14970 | − | Transgenic |
| | | | | | insert |
| TI-1875 | CTTG | FnCas12a | 14957:14979 | − | Transgenic |
| | | | | | insert |
| TI-1876 | TTTA | LbCas12a | 14967:14989 | − | Transgenic |
| | | | | | insert |
| TI-1877 | GTTA | FnCas12a | 14972:14994 | − | Transgenic |
| | | | | | insert |
| TI-1878 | TTTA | LbCas12a-TYC | 15022:15044 | − | Transgenic |
| | | | | | insert |
| TI-1879 | GTTA | FnCas12a | 15047:15069 | + | Transgenic |
| | | | | | insert |
| TI-1880 | TATG | LbCas12a-TATV | 15027:15049 | − | Transgenic |
| | | | | | insert |
| TI-1881 | TTTA | LbCas12a | 15029:15051 | − | Transgenic |
| | | | | | insert |
| TI-1882 | TTTA | LbCas12a-TYC | 15049:15071 | − | Transgenic |
| | | | | | insert |
| TI-1883 | TTTA | LbCas12a | 15056:15078 | − | Transgenic |
| | | | | | insert |
| TI-1884 | TTTC | LbCas12a | 15105:15127 | + | Transgenic |
| | | | | | insert |
| TI-1885 | TTCA | LbCas12a-TYC | 15106:15128 | + | Transgenic |
| | | | | | insert |
| TI-1886 | TTTA | LbCas12a-TYC | 15091:15113 | − | Transgenic |
| | | | | | insert |
| TI-1887 | TATA | LbCas12a-TATV | 15096:15118 | − | Transgenic |
| | | | | | insert |
| TI-1888 | TATA | LbCas12a-TATV | 15123:15145 | + | Transgenic |
| | | | | | insert |
| TI-1889 | TATG | LbCas12a-TATV | 15142:15164 | + | Transgenic |
| | | | | | insert |
| TI-1890 | TTTG | LbCas12a | 15123:15145 | − | Transgenic |
| | | | | | insert |
| TI-1891 | TATG | LbCas12a-TATV | 15146:15168 | + | Transgenic |
| | | | | | insert |
| TI-1892 | TTCA | LbCas12a-TYC | 15158:15180 | + | Transgenic |
| | | | | | insert |
| TI-1893 | TCCA | LbCas12a-TYC | 15146:15168 | − | Transgenic |
| | | | | | insert |
| TI-1894 | TTCC | LbCas12a-TYC | 15150:15172 | − | Transgenic |
| | | | | | insert |
| TI-1895 | TTTA | LbCas12a | 15212:15234 | + | Transgenic |
| | | | | | insert |
| TI-1896 | TTCA | LbCas12a-TYC | 15214:15236 | − | Transgenic |
| | | | | | insert |
| TI-1897 | TTTC | LbCas12a | 15215:15237 | − | Transgenic |
| | | | | | insert |
| TI-1898 | TATG | LbCas12a-TATV | 15251:15273 | − | Transgenic |
| | | | | | insert |
| TI-1899 | TATG | LbCas12a-TATV | 15280:15302 | + | Transgenic |
| | | | | | insert |
| TI-1900 | CTTC | FnCas12a | 15295:15317 | + | Transgenic |
| | | | | | insert |
| TI-1901 | CTTG | FnCas12a | 15298:15320 | + | Transgenic |
| | | | | | insert |
| TI-1902 | TATC | LbCas12a-TATV | 15278:15300 | − | Transgenic |
| | | | | | insert |
| TI-1903 | TTTG | LbCas12a | 15288:15310 | − | Transgenic |
| | | | | | insert |
| TI-1904 | TTTG | LbCas12a | 15311:15333 | + | Transgenic |
| | | | | | insert |
| TI-1905 | TTCG | LbCas12a-TYC | 15300:15322 | − | Transgenic |
| | | | | | insert |
| TI-1906 | GTTC | FnCas12a | 15301:15323 | − | Transgenic |
| | | | | | insert |
| TI-1907 | TCCG | LbCas12a-TYC | 15323:15345 | + | Transgenic |
| | | | | | insert |
| TI-1908 | CTTC | FnCas12a | 15331:15353 | + | Transgenic |
| | | | | | insert |
| TI-1909 | GTTA | FnCas12a | 15309:15331 | − | Transgenic |
| | | | | | insert |
| TI-1910 | CTTA | FnCas12a | 15334:15356 | + | Transgenic |
| | | | | | insert |
| TI-1911 | TCCC | LbCas12a-TYC | 15344:15366 | + | Transgenic |
| | | | | | insert |
| TI-1912 | TCCG | LbCas12a-TYC | 15350:15372 | + | Transgenic |
| | | | | | insert |
| TI-1913 | TCCG | LbCas12a-TYC | 15396:15418 | + | Transgenic |
| | | | | | insert |
| TI-1914 | TCCA | LbCas12a-TYC | 15383:15405 | − | Transgenic |
| | | | | | insert |
| TI-1915 | CTTG | FnCas12a | 15421:15443 | + | Transgenic |
| | | | | | insert |
| TI-1916 | TCCA | LbCas12a-TYC | 15429:15451 | + | Transgenic |
| | | | | | insert |
| TI-1917 | TATC | LbCas12a-TATV | 15437:15459 | + | Transgenic |
| | | | | | insert |
| TI-1918 | TTCG | LbCas12a-TYC | 15422:15444 | − | Transgenic |
| | | | | | insert |
| TI-1919 | CTTC | FnCas12a | 15423:15445 | − | Transgenic |
| | | | | | insert |
| TI-1920 | TTCG | LbCas12a-TYC | 15435:15457 | − | Transgenic |
| | | | | | insert |
| TI-1921 | GTTC | FnCas12a | 15436:15458 | − | Transgenic |
| | | | | | insert |
| TI-1922 | GTTG | FnCas12a | 15448:15470 | − | Transgenic |
| | | | | | insert |
| TI-1923 | TTTC | LbCas12a | 15509:15531 | + | Transgenic |
| | | | | | insert |
| TI-1924 | TTCG | LbCas12a-TYC | 15510:15532 | + | Transgenic |
| | | | | | insert |
| TI-1925 | TATG | LbCas12a-TATV | 15489:15511 | − | Transgenic |
| | | | | | insert |
| TI-1926 | CTTA | FnCas12a | 15491:15513 | − | Transgenic |
| | | | | | insert |
| TI-1927 | TTTG | LbCas12a | 15524:15546 | + | Transgenic |
| | | | | | insert |
| TI-1928 | TCCC | LbCas12a-TYC | 15505:15527 | − | Transgenic |
| | | | | | insert |
| TI-1929 | CTTC | FnCas12a | 15541:15563 | + | Transgenic |
| | | | | | insert |
| TI-1930 | CTTC | FnCas12a | 15550:15572 | + | Transgenic |
| | | | | | insert |
| TI-1931 | TTCC | LbCas12a-TYC | 15551:15573 | + | Transgenic |
| | | | | | insert |
| TI-1932 | CTTG | FnCas12a | 15554:15576 | + | Transgenic |
| | | | | | insert |
| TI-1933 | TTTA | LbCas12a | 15566:15588 | + | Transgenic |
| | | | | | insert |
| TI-1934 | CTTC | FnCas12a | 15547:15569 | − | Transgenic |
| | | | | | insert |
| TI-1935 | GTTC | FnCas12a | 15550:15572 | − | Transgenic |
| | | | | | insert |
| TI-1936 | CTTG | FnCas12a | 15584:15606 | + | Transgenic |
| | | | | | insert |
| TI-1937 | GTTG | FnCas12a | 15592:15614 | + | Transgenic |
| | | | | | insert |
| TI-1938 | GTTC | FnCas12a | 15595:15617 | + | Transgenic |
| | | | | | insert |
| TI-1939 | TTCA | LbCas12a-TYC | 15596:15618 | + | Transgenic |
| | | | | | insert |
| TI-1940 | TCCA | LbCas12a-TYC | 15605:15627 | + | Transgenic |
| | | | | | insert |
| TI-1941 | TTTC | LbCas12a | 15611:15633 | + | Transgenic |
| | | | | | insert |
| TI-1942 | CTTG | FnCas12a | 15616:15638 | + | Transgenic |
| | | | | | insert |
| TI-1943 | TTCA | LbCas12a-TYC | 15594:15616 | − | Transgenic |
| | | | | | insert |
| TI-1944 | GTTC | FnCas12a | 15595:15617 | − | Transgenic |
| | | | | | insert |
| TI-1945 | CTTG | FnCas12a | 15625:15647 | + | Transgenic |
| | | | | | insert |
| TI-1946 | CTTG | FnCas12a | 15603:15625 | − | Transgenic |
| | | | | | insert |
| TI-1947 | TTCA | LbCas12a-TYC | 15615:15637 | − | Transgenic |
| | | | | | insert |
| TI-1948 | CTTC | FnCas12a | 15616:15638 | − | Transgenic |
| | | | | | insert |
| TI-1949 | GTTG | FnCas12a | 15640:15662 | + | Transgenic |
| | | | | | insert |
| TI-1950 | GTTA | FnCas12a | 15626:15648 | − | Transgenic |
| | | | | | insert |
| TI-1951 | TTCG | LbCas12a-TYC | 15629:15651 | − | Transgenic |
| | | | | | insert |
| TI-1952 | GTTC | FnCas12a | 15697:15719 | + | Transgenic |
| | | | | | insert |
| TI-1953 | TTCA | LbCas12a-TYC | 15698:15720 | + | Transgenic |
| | | | | | insert |
| TI-1954 | TCCC | LbCas12a-TYC | 15707:15729 | + | Transgenic |
| | | | | | insert |
| TI-1955 | TTCG | LbCas12a-TYC | 15736:15758 | + | Transgenic |
| | | | | | insert |
| TI-1956 | CTTC | FnCas12a | 15742:15764 | + | Transgenic |
| | | | | | insert |
| TI-1957 | CTTC | FnCas12a | 15748:15770 | + | Transgenic |
| | | | | | insert |
| TI-1958 | TTCA | LbCas12a-TYC | 15749:15771 | + | Transgenic |
| | | | | | insert |
| TI-1959 | TTCA | LbCas12a-TYC | 15742:15764 | − | Transgenic |
| | | | | | insert |
| TI-1960 | CTTC | FnCas12a | 15743:15765 | − | Transgenic |
| | | | | | insert |
| TI-1961 | TATG | LbCas12a-TATV | 15767:15789 | + | Transgenic |
| | | | | | insert |
| TI-1962 | TCCC | LbCas12a-TYC | 15794:15816 | + | Transgenic |
| | | | | | insert |
| TI-1963 | CTTG | FnCas12a | 15787:15809 | − | Transgenic |
| | | | | | insert |
| TI-1964 | CTTC | FnCas12a | 15820:15842 | + | Transgenic |
| | | | | | insert |
| TI-1965 | TTCA | LbCas12a-TYC | 15821:15843 | + | Transgenic |
| | | | | | insert |
| TI-1966 | GTTG | FnCas12a | 15807:15829 | − | Transgenic |
| | | | | | insert |
| TI-1967 | TTCG | LbCas12a-TYC | 15810:15832 | − | Transgenic |
| | | | | | insert |
| TI-1968 | TATG | LbCas12a-TATV | 15846:15868 | + | Transgenic |
| | | | | | insert |
| TI-1969 | TCCA | LbCas12a-TYC | 15851:15873 | + | Transgenic |
| | | | | | insert |
| TI-1970 | CTTA | FnCas12a | 15837:15859 | − | Transgenic |
| | | | | | insert |
| TI-1971 | TCCG | LbCas12a-TYC | 15846:15868 | − | Transgenic |
| | | | | | insert |
| TI-1972 | TTTC | LbCas12a | 15880:15902 | + | Transgenic |
| | | | | | insert |
| TI-1973 | TTCA | LbCas12a-TYC | 15881:15903 | + | Transgenic |
| | | | | | insert |
| TI-1974 | CTTC | FnCas12a | 15886:15908 | + | Transgenic |
| | | | | | insert |
| TI-1975 | TTCA | LbCas12a-TYC | 15887:15909 | + | Transgenic |
| | | | | | insert |
| TI-1976 | TTCA | LbCas12a-TYC | 15870:15892 | − | Transgenic |
| | | | | | insert |
| TI-1977 | GTTC | FnCas12a | 15871:15893 | − | Transgenic |
| | | | | | insert |
| TI-1978 | CTTG | FnCas12a | 15895:15917 | + | Transgenic |
| | | | | | insert |
| TI-1979 | TTCG | LbCas12a-TYC | 15874:15896 | − | Transgenic |
| | | | | | insert |
| TI-1980 | CTTC | FnCas12a | 15875:15897 | − | Transgenic |
| | | | | | insert |
| TI-1981 | CTTA | FnCas12a | 15910:15932 | + | Transgenic |
| | | | | | insert |
| TI-1982 | TCCC | LbCas12a-TYC | 15890:15912 | − | Transgenic |
| | | | | | insert |
| TI-1983 | TTCC | LbCas12a-TYC | 15891:15913 | − | Transgenic |
| | | | | | insert |
| TI-1984 | GTTC | FnCas12a | 15892:15914 | − | Transgenic |
| | | | | | insert |
| TI-1985 | CTTC | FnCas12a | 15902:15924 | − | Transgenic |
| | | | | | insert |
| TI-1986 | TCCA | LbCas12a-TYC | 15908:15930 | − | Transgenic |
| | | | | | insert |
| TI-1987 | TTCA | LbCas12a-TYC | 15918:15940 | − | Transgenic |
| | | | | | insert |
| TI-1988 | CTTC | FnCas12a | 15919:15941 | − | Transgenic |
| | | | | | insert |
| TI-1989 | TCCG | LbCas12a-TYC | 15963:15985 | − | Transgenic |
| | | | | | insert |
| TI-1990 | GTTC | FnCas12a | 15967:15989 | − | Transgenic |
| | | | | | insert |
| TI-1991 | TTCA | LbCas12a-TYC | 16011:16033 | − | Transgenic |
| | | | | | insert |
| TI-1992 | TTTC | LbCas12a | 16012:16034 | − | Transgenic |
| | | | | | insert |
| TI-1993 | TTTA | LbCas12a | 16019:16041 | − | Transgenic |
| | | | | | insert |
| TI-1994 | TTTG | LbCas12a | 16061:16083 | + | Transgenic |
| | | | | | insert |
| TI-1995 | CTTG | FnCas12a | 16064:16086 | − | Transgenic |
| | | | | | insert |
| TI-1996 | TTTC | LbCas12a | 16067:16089 | − | Transgenic |
| | | | | | insert |
| TI-1997 | TTTC | LbCas12a | 16112:16134 | + | Transgenic |
| | | | | | insert |
| TI-1998 | TTCA | LbCas12a-TYC | 16113:16135 | + | Transgenic |
| | | | | | insert |
| TI-1999 | GTTG | FnCas12a | 16127:16149 | + | Transgenic |
| | | | | | insert |
| TI-2000 | TTTG | LbCas12a | 16135:16157 | + | Transgenic |
| | | | | | insert |
| TI-2001 | CTTA | FnCas12a | 16118:16140 | − | Transgenic |
| | | | | | insert |
| TI-2002 | TCCA | LbCas12a-TYC | 16140:16162 | + | Transgenic |
| | | | | | insert |
| TI-2003 | TATC | LbCas12a-TATV | 16130:16152 | − | Transgenic |
| | | | | | insert |
| TI-2004 | TCCA | LbCas12a-TYC | 16137:16159 | − | Transgenic |
| | | | | | insert |
| TI-2005 | TTTC | LbCas12a | 16141:16163 | − | Transgenic |
| | | | | | insert |
| TI-2006 | TTTC | LbCas12a | 16178:16200 | + | Transgenic |
| | | | | | insert |
| TI-2007 | TTCA | LbCas12a-TYC | 16179:16201 | + | Transgenic |
| | | | | | insert |
| TI-2008 | TTTG | LbCas12a | 16185:16207 | + | Transgenic |
| | | | | | insert |
| TI-2009 | TTTG | LbCas12a | 16176:16198 | − | Transgenic |
| | | | | | insert |
| TI-2010 | TTTG | LbCas12a | 16186:16208 | − | Transgenic |
| | | | | | insert |
| TI-2011 | TTTG | LbCas12a | 16204:16226 | − | Transgenic |
| | | | | | insert |
| TI-2012 | TCCG | LbCas12a-TYC | 16243:16265 | + | Transgenic |
| | | | | | insert |
| TI-2013 | TTCA | LbCas12a-TYC | 16233:16255 | − | Transgenic |
| | | | | | insert |
| TI-2014 | TATA | LbCas12a-TATV | 16255:16277 | + | Transgenic |
| | | | | | insert |
| TI-2015 | TTTG | LbCas12a | 16240:16262 | − | Transgenic |
| | | | | | insert |
| TI-2016 | TTCG | LbCas12a-TYC | 16253:16275 | − | Transgenic |
| | | | | | insert |
| TI-2017 | TATA | LbCas12a-TATV | 16259:16281 | − | Transgenic |
| | | | | | insert |
| TI-2018 | TTTA | LbCas12a | 16261:16283 | − | Transgenic |
| | | | | | insert |
| TI-2019 | TATA | LbCas12a-TATV | 16286:16308 | + | Transgenic |
| | | | | | insert |
| TI-2020 | GTTC | FnCas12a | 16303:16325 | + | Transgenic |
| | | | | | insert |
| TI-2021 | TTTG | LbCas12a | 16282:16304 | − | Transgenic |
| | | | | | insert |
| TI-2022 | TCCA | LbCas12a-TYC | 16307:16329 | + | Transgenic |
| | | | | | insert |
| TI-2023 | TTTG | LbCas12a | 16288:16310 | − | Transgenic |
| | | | | | insert |
| TI-2024 | TTTC | LbCas12a | 16295:16317 | − | Transgenic |
| | | | | | insert |
| TI-2025 | TCCA | LbCas12a-TYC | 16316:16338 | − | Transgenic |
| | | | | | insert |
| TI-2026 | TTCC | LbCas12a-TYC | 16317:16339 | − | Transgenic |
| | | | | | insert |
| TI-2027 | TTTC | LbCas12a | 16318:16340 | − | Transgenic |
| | | | | | insert |
| TI-2028 | TTTA | LbCas12a | 16326:16348 | − | Transgenic |
| | | | | | insert |
| TI-2029 | TCCC | LbCas12a-TYC | 16359:16381 | − | Transgenic |
| | | | | | insert |
| TI-2030 | TTTC | LbCas12a | 16404:16426 | + | Transgenic |
| | | | | | insert |
| TI-2031 | GTTG | FnCas12a | 16417:16439 | + | Transgenic |
| | | | | | insert |
| TI-2032 | CTTG | FnCas12a | 16404:16426 | − | Transgenic |
| | | | | | insert |
| TI-2033 | TTTC | LbCas12a | 16427:16449 | + | Transgenic |
| | | | | | insert |
| TI-2034 | TTCC | LbCas12a-TYC | 16428:16450 | + | Transgenic |
| | | | | | insert |
| TI-2035 | TCCA | LbCas12a-TYC | 16429:16451 | + | Transgenic |
| | | | | | insert |
| TI-2036 | GTTA | FnCas12a | 16438:16460 | + | Transgenic |
| | | | | | insert |
| TI-2037 | TATG | LbCas12a-TATV | 16440:16462 | + | Transgenic |
| | | | | | insert |
| TI-2038 | TCCG | LbCas12a-TYC | 16457:16479 | + | Transgenic |
| | | | | | insert |
| TI-2039 | TTTA | LbCas12a | 16464:16486 | + | Transgenic |
| | | | | | insert |
| TI-2040 | GTTG | FnCas12a | 16475:16497 | + | Transgenic |
| | | | | | insert |
| TI-2041 | TTTC | LbCas12a | 16465:16487 | − | Transgenic |
| | | | | | insert |
| TI-2042 | TATA | LbCas12a-TATV | 16469:16491 | − | Transgenic |
| | | | | | insert |
| TI-2043 | TATA | LbCas12a-TATV | 16496:16518 | + | Transgenic |
| | | | | | insert |
| TI-2044 | TATA | LbCas12a-TATV | 16478:16500 | − | Transgenic |
| | | | | | insert |
| TI-2045 | TATA | LbCas12a-TATV | 16480:16502 | − | Transgenic |
| | | | | | insert |
| TI-2046 | TATA | LbCas12a-TATV | 16482:16504 | − | Transgenic |
| | | | | | insert |
| TI-2047 | TATA | LbCas12a-TATV | 16505:16527 | + | Transgenic |
| | | | | | insert |
| TI-2048 | TATA | LbCas12a-TATV | 16484:16506 | − | Transgenic |
| | | | | | insert |
| TI-2049 | TATA | LbCas12a-TATV | 16507:16529 | + | Transgenic |
| | | | | | insert |
| TI-2050 | TATA | LbCas12a-TATV | 16509:16531 | + | Transgenic |
| | | | | | insert |
| TI-2051 | TCCC | LbCas12a-TYC | 16488:16510 | − | Transgenic |
| | | | | | insert |
| TI-2052 | TTCC | LbCas12a-TYC | 16489:16511 | − | Transgenic |
| | | | | | insert |
| TI-2053 | TATA | LbCas12a-TATV | 16511:16533 | + | Transgenic |
| | | | | | insert |
| TI-2054 | GTTC | FnCas12a | 16490:16512 | − | Transgenic |
| | | | | | insert |
| TI-2055 | TTTA | LbCas12a | 16535:16557 | + | Transgenic |
| | | | | | insert |
| TI-2056 | GTTG | FnCas12a | 16547:16569 | + | Transgenic |
| | | | | | insert |
| TI-2057 | TTTA | LbCas12a | 16559:16581 | + | Transgenic |
| | | | | | insert |
| TI-2058 | TTTA | LbCas12a | 16564:16586 | + | Transgenic |
| | | | | | insert |
| TI-2059 | TTTA | LbCas12a | 16545:16567 | − | Transgenic |
| | | | | | insert |
| TI-2060 | TTTG | LbCas12a | 16552:16574 | − | Transgenic |
| | | | | | insert |
| TI-2061 | CTTA | FnCas12a | 16585:16607 | + | Transgenic |
| | | | | | insert |
| TI-2062 | TATC | LbCas12a-TATV | 16569:16591 | − | Transgenic |
| | | | | | insert |
| TI-2063 | TTTA | LbCas12a | 16571:16593 | − | Transgenic |
| | | | | | insert |
| TI-2064 | TATG | LbCas12a-TATV | 16582:16604 | − | Transgenic |
| | | | | | insert |
| TI-2065 | TTTA | LbCas12a | 16629:16651 | + | Transgenic |
| | | | | | insert |
| TI-2066 | CTTA | FnCas12a | 16638:16660 | + | Transgenic |
| | | | | | insert |
| TI-2067 | TATA | LbCas12a-TATV | 16630:16652 | − | Transgenic |
| | | | | | insert |
| TI-2068 | TTTA | LbCas12a | 16632:16654 | − | Transgenic |
| | | | | | insert |
| TI-2069 | TTTA | FnCas12a | 16664:16686 | + | Transgenic |
| | | | | | insert |
| TI-2070 | TCCA | LbCas12a-TYC | 16669:16691 | + | Transgenic |
| | | | | | insert |
| TI-2071 | TTTG | LbCas12a | 16683:16705 | + | Transgenic |
| | | | | | insert |
| TI-2072 | TATA | LbCas12a-TATV | 16676:16698 | − | Transgenic |
| | | | | | insert |
| TI-2073 | TATA | LbCas12a-TATV | 16678:16700 | − | Transgenic |
| | | | | | insert |
| TI-2074 | TATA | LbCas12a-TATV | 16680:16702 | − | Transgenic |
| | | | | | insert |
| TI-2075 | TATA | LbCas12a-TATV | 16703:16725 | + | Transgenic |
| | | | | | insert |
| TI-2076 | TATA | LbCas12a-TATV | 16705:16727 | + | Transgenic |
| | | | | | insert |
| TI-2077 | TATA | LbCas12a-TATV | 16707:16729 | + | Transgenic |
| | | | | | insert |
| TI-2078 | TATA | LbCas12a-TATV | 16712:16734 | + | Transgenic |
| | | | | | insert |
| TI-2079 | TATG | LbCas12a-TATV | 16714:16736 | + | Transgenic |
| | | | | | insert |
| TI-2080 | TTTC | LbCas12a | 16720:16742 | + | Transgenic |
| | | | | | insert |
| TI-2081 | TTCA | LbCas12a-TYC | 16705:16727 | − | Transgenic |
| | | | | | insert |
| TI-2082 | TTTC | LbCas12a | 16706:16728 | − | Transgenic |
| | | | | | insert |
| TI-2083 | TATG | LbCas12a-TATV | 16717:16739 | − | Transgenic |
| | | | | | insert |
| TI-2084 | TTTC | LbCas12a | 16721:16743 | − | Transgenic |
| | | | | | insert |
| TI-2085 | TCCC | LbCas12a-TYC | 16727:16749 | − | Transgenic |
| | | | | | insert |
| TI-2086 | TTCC | LbCas12a-TYC | 16728:16750 | − | Transgenic |
| | | | | | insert |
| TI-2087 | TTTC | LbCas12a | 16729:16751 | − | Transgenic |
| | | | | | insert |
| TI-2088 | TTTA | LbCas12a | 16764:16786 | + | Transgenic |
| | | | | | insert |
| TI-2089 | TATC | LbCas12a-TATV | 16766:16788 | + | Transgenic |
| | | | | | insert |
| TI-2090 | TTTG | LbCas12a | 16771:16793 | + | Transgenic |
| | | | | | insert |
| TI-2091 | GTTC | FnCas12a | 16774:16796 | + | Transgenic |
| | | | | | insert |
| TI-2092 | CTTA | FnCas12a | 16774:16796 | − | Transgenic |
| | | | | | insert |
| TI-2093 | TTTG | LbCas12a | 16810:16832 | + | Transgenic |
| | | | | | insert |
| TI-2094 | TTTC | LbCas12a-TYC | 16814:16836 | + | Transgenic |
| | | | | | insert |
| TI-2095 | CTTG | FnCas12a | 16793:16815 | − | Transgenic |
| | | | | | insert |
| TI-2096 | TTCA | LbCas12a-TYC | 16815:16837 | + | Transgenic |
| | | | | | insert |
| TI-2097 | TTTC | LbCas12a | 16796:16818 | − | Transgenic |
| | | | | | insert |
| TI-2098 | TATG | LbCas12a-TATV | 16814:16836 | − | Transgenic |
| | | | | | insert |
| TI-2099 | TTTA | LbCas12a | 16816:16838 | − | Transgenic |
| | | | | | insert |
| TI-2100 | TTTA | LbCas12a-TYC | 16853:16875 | + | Transgenic |
| | | | | | insert |
| TI-2101 | TTTG | LbCas12a | 16849:16871 | − | Transgenic |
| | | | | | insert |
| TI-2102 | TATA | LbCas12a-TATV | 16897:16919 | + | Transgenic |
| | | | | | insert |
| TI-2103 | CTTA | FnCas12a | 16915:16937 | + | Transgenic |
| | | | | | insert |
| TI-2104 | TTTG | LbCas12a | 16920:16942 | + | Transgenic |
| | | | | | insert |
| TI-2105 | GTTG | FnCas12a | 16927:16949 | + | Transgenic |
| | | | | | insert |
| TI-2106 | TATG | LbCas12a-TATV | 16935:16957 | + | Transgenic |
| | | | | | insert |
| TI-2107 | TCCG | LbCas12a-TYC | 16919:16941 | − | Transgenic |
| | | | | | insert |
| TI-2108 | GTTC | FnCas12a | 16943:16965 | + | Transgenic |
| | | | | | insert |
| TI-2109 | TATC | LbCas12a-TATV | 16921:16943 | − | Transgenic |
| | | | | | insert |
| TI-2110 | TTCG | LbCas12a-TYC | 16944:16966 | + | Transgenic |
| | | | | | insert |
| TI-2111 | GTTA | FnCas12a | 16923:16945 | − | Transgenic |
| | | | | | insert |
| TI-2112 | GTTA | FnCas12a | 16929:16951 | − | Transgenic |
| | | | | | insert |
| TI-2113 | TCCA | LbCas12a-TYC | 16947:16969 | − | Transgenic |
| | | | | | insert |
| TI-2114 | TATC | LbCas12a-TATV | 16949:16971 | − | Transgenic |
| | | | | | insert |
| TI-2115 | CTTA | FnCas12a | 16951:16973 | − | Transgenic |
| | | | | | insert |
| TI-2116 | TCCC | LbCas12a-TYC | 16967:16989 | − | Transgenic |
| | | | | | insert |
| TI-2117 | GTTG | FnCas12a | 17007:17029 | + | Transgenic |
| | | | | | insert |
| TI-2118 | TCCA | LbCas12a-TYC | 16986:17008 | − | Transgenic |
| | | | | | insert |
| TI-2119 | GTTG | FnCas12a | 17011:17033 | + | Transgenic |
| | | | | | insert |
| TI-2120 | TTTC | LbCas12a | 17018:17040 | + | Transgenic |
| | | | | | insert |
| TI-2121 | TTCC | LbCas12a-TYC | 17019:17041 | + | Transgenic |
| | | | | | insert |
| TI-2122 | CTTA | FnCas12a | 17022:17044 | + | Transgenic |
| | | | | | insert |
| TI-2123 | TATC | LbCas12a-TATV | 17024:17046 | + | Transgenic |
| | | | | | insert |
| TI-2124 | TCCA | LbCas12a-TYC | 17026:17048 | + | Transgenic |
| | | | | | insert |
| TI-2125 | GTTG | FnCas12a | 17036:17058 | + | Transgenic |
| | | | | | insert |
| TI-2126 | TTTG | LbCas12a | 17044:17066 | + | Transgenic |
| | | | | | insert |
| TI-2127 | TTTG | LbCas12a | 17052:17074 | + | Transgenic |
| | | | | | insert |
| TI-2128 | GTTC | FnCas12a | 17061:17083 | + | Transgenic |
| | | | | | insert |
| TI-2129 | TTTG | LbCas12a | 17066:17088 | + | Transgenic |
| | | | | | insert |
| TI-2130 | CTTA | FnCas12a | 17070:17092 | + | Transgenic |
| | | | | | insert |
| TI-2131 | TATC | LbCas12a-TATV | 17072:17094 | + | Transgenic |
| | | | | | insert |
| TI-2132 | TATC | LbCas12a-TATV | 17053:17075 | − | Transgenic |
| | | | | | insert |
| TI-2133 | TTTG | LbCas12a | 17077:17099 | + | Transgenic |
| | | | | | insert |
| TI-2134 | CTTA | FnCas12a | 17055:17077 | − | Transgenic |
| | | | | | insert |
| TI-2135 | TTCA | LbCas12a-TYC | 17063:17085 | − | Transgenic |
| | | | | | insert |
| TI-2136 | TTTC | LbCas12a | 17064:17086 | − | Transgenic |
| | | | | | insert |
| TI-2137 | GTTG | FnCas12a | 17101:17123 | + | Transgenic |
| | | | | | insert |
| TI-2138 | TATC | LbCas12a-TATV | 17116:17138 | + | Transgenic |
| | | | | | insert |
| TI-2139 | CTTA | FnCas12a | 17119:17141 | + | Transgenic |
| | | | | | insert |
| TI-2140 | TATC | LbCas12a-TATV | 17101:17123 | − | Transgenic |
| | | | | | insert |
| TI-2141 | TTTG | LbCas12a | 17125:17147 | + | Transgenic |
| | | | | | insert |
| TI-2142 | TCCA | LbCas12a-TYC | 17111:17133 | − | Transgenic |
| | | | | | insert |
| TI-2143 | TATC | LbCas12a-TATV | 17113:17135 | − | Transgenic |
| | | | | | insert |
| TI-2144 | TTTA | LbCas12a | 17126:17148 | − | Transgenic |
| | | | | | insert |
| TI-2145 | CTTA | FnCas12a | 17148:17170 | + | Transgenic |
| | | | | | insert |
| TI-2146 | TTTG | LbCas12a | 17160:17182 | + | Transgenic |
| | | | | | insert |
| TI-2147 | CTTG | FnCas12a | 17140:17162 | − | Transgenic |
| | | | | | insert |
| TI-2148 | TTTA | LbCas12a | 17173:17195 | + | Transgenic |
| | | | | | insert |
| TI-2149 | TATC | LbCas12a-TATV | 17175:17197 | + | Transgenic |
| | | | | | insert |
| TI-2150 | TTTC | LbCas12a | 17189:17211 | + | Transgenic |
| | | | | | insert |
| TI-2151 | CTTA | FnCas12a | 17192:17214 | + | Transgenic |
| | | | | | insert |
| TI-2152 | TTCC | LbCas12a-TYC | 17196:17218 | + | Transgenic |
| | | | | | insert |
| TI-2153 | CTTA | FnCas12a | 17199:17221 | + | Transgenic |
| | | | | | insert |
| TI-2154 | TATG | LbCas12a-TATV | 17207:17229 | + | Transgenic |
| | | | | | insert |
| TI-2155 | GTTA | FnCas12a | 17214:17236 | + | Transgenic |
| | | | | | insert |
| TI-2156 | TTTC | LbCas12a | 17219:17241 | + | Transgenic |
| | | | | | insert |
| TI-2157 | TTCC | LbCas12a-TYC | 17220:17242 | + | Transgenic |
| | | | | | insert |
| TI-2158 | GTTC | FnCas12a | 17226:17248 | + | Transgenic |
| | | | | | insert |
| TI-2159 | TTCA | LbCas12a-TYC | 17227:17249 | + | Transgenic |
| | | | | | insert |
| TI-2160 | TTTC | LbCas12a-TYC | 17235:17257 | + | Transgenic |
| | | | | | insert |
| TI-2161 | CTTA | FnCas12a | 17238:17260 | + | Transgenic |
| | | | | | insert |
| TI-2162 | TTCA | LbCas12a-TYC | 17219:17241 | − | Transgenic |
| | | | | | insert |
| TI-2163 | TTTC | LbCas12a | 17220:17242 | − | Transgenic |
| | | | | | insert |
| TI-2164 | TATA | LbCas12a-TATV | 17226:17248 | − | Transgenic |
| | | | | | insert |
| TI-2165 | TTTA | LbCas12a | 17237:17259 | − | Transgenic |
| | | | | | insert |
| TI-2166 | TTCA | LbCas12a-TYC | 17312:17334 | + | Transgenic |
| | | | | | insert |
| TI-2167 | TTTG | LbCas12a | 17317:17339 | + | Transgenic |
| | | | | | insert |
| TI-2168 | TTTA | LbCas12a | 17307:17329 | − | Transgenic |
| | | | | | insert |
| TI-2169 | TTTG | LbCas12a-TYC | 17321:17343 | − | Transgenic |
| | | | | | insert |
| TI-2170 | TATA | LbCas12a-TATV | 17354:17376 | + | Transgenic |
| | | | | | insert |
| TI-2171 | TATA | LbCas12a-TATV | 17356:17378 | + | Transgenic |
| | | | | | insert |
| TI-2172 | TATA | LbCas12a-TATV | 17358:17380 | + | Transgenic |
| | | | | | insert |
| TI-2173 | CTTG | FnCas12a | 17367:17389 | + | Transgenic |
| | | | | | insert |
| TI-2174 | TATG | LbCas12a-TATV | 17371:17393 | + | Transgenic |
| | | | | | insert |
| TI-2175 | TCCC | LbCas12a-TYC | 17364:17386 | − | Transgenic |
| | | | | | insert |
| TI-2176 | TATC | LbCas12a-TATV | 17366:17388 | − | Transgenic |
| | | | | | insert |
| TI-2177 | GTTA | FnCas12a | 17368:17390 | − | Transgenic |
| | | | | | insert |
| TI-2178 | TTTG | LbCas12a | 17393:17415 | − | Transgenic |
| | | | | | insert |
| TI-2179 | GTTG | FnCas12a | 17408:17430 | − | Transgenic |
| | | | | | insert |
| TI-2180 | TATA | LbCas12a-TATV | 17415:17437 | − | Transgenic |
| | | | | | insert |
| TI-2181 | GTTA | FnCas12a | 17440:17462 | + | Transgenic |
| | | | | | insert |
| TI-2182 | TATA | LbCas12a-TATV | 17442:17464 | + | Transgenic |
| | | | | | insert |
| TI-2183 | TTCG | LbCas12a-TYC | 17423:17445 | − | Transgenic |
| | | | | | insert |
| TI-2184 | CTTC | FnCas12a | 17424:17446 | − | Transgenic |
| | | | | | insert |
| TI-2185 | GTTA | FnCas12a | 17446:17468 | + | Transgenic |
| | | | | | insert |
| TI-2186 | TTCA | LbCas12a-TYC | 17434:17456 | − | Transgenic |
| | | | | | insert |
| TI-2187 | TTTC | LbCas12a | 17435:17457 | − | Transgenic |
| | | | | | insert |
| TI-2188 | TCCC | LbCas12a-TYC | 17457:17479 | + | Transgenic |
| | | | | | insert |
| TI-2189 | TCCA | LbCas12a-TYC | 17446:17468 | − | Transgenic |
| | | | | | insert |
| TI-2190 | GTTG | FnCas12a | 17471:17493 | + | Transgenic |
| | | | | | insert |
| TI-2191 | GTTA | FnCas12a | 17453:17475 | − | Transgenic |
| | | | | | insert |
| TI-2192 | GTTA | FnCas12a | 17478:17500 | + | Transgenic |
| | | | | | insert |
| TI-2193 | TTTG | LbCas12a | 17462:17484 | − | Transgenic |
| | | | | | insert |
| TI-2194 | TTTC | LbCas12a | 17499:17521 | + | Transgenic |
| | | | | | insert |
| TI-2195 | CTTA | FnCas12a | 17502:17524 | + | Transgenic |
| | | | | | insert |
| TI-2196 | TATC | LbCas12a-TATV | 17504:17526 | + | Transgenic |
| | | | | | insert |
| TI-2197 | CTTA | FnCas12a | 17492:17514 | − | Transgenic |
| | | | | | insert |
| TI-2198 | TTTG | LbCas12a | 17533:17555 | + | Transgenic |
| | | | | | insert |
| TI-2199 | CTTG | FnCas12a | 17544:17566 | + | Transgenic |
| | | | | | insert |
| TI-2200 | GTTG | FnCas12a | 17563:17585 | + | Transgenic |
| | | | | | insert |
| TI-2201 | CTTG | FnCas12a | 17554:17576 | − | Transgenic |
| | | | | | insert |
| TI-2202 | TCCA | LbCas12a-TYC | 17559:17581 | − | Transgenic |
| | | | | | insert |
| TI-2203 | CTTA | FnCas12a | 17625:17647 | − | Transgenic |
| | | | | | insert |
| TI-2204 | TTCG | LbCas12a-TYC | 17631:17653 | − | Transgenic |
| | | | | | insert |
| TI-2205 | TTTG | LbCas12a | 17662:17684 | + | Transgenic |
| | | | | | insert |
| TI-2206 | TTTC | LbCas12a | 17689:17711 | + | Transgenic |
| | | | | | insert |
| TI-2207 | TTTG | LbCas12a | 17672:17694 | − | Transgenic |
| | | | | | insert |
| TI-2208 | TTTC | LbCas12a | 17696:17718 | + | Transgenic |
| | | | | | insert |
| TI-2209 | TTCA | LbCas12a-TYC | 17697:17719 | + | Transgenic |
| | | | | | insert |
| TI-2210 | TTCG | LbCas12a-TYC | 17706:17728 | + | Transgenic |
| | | | | | insert |
| TI-2211 | GTTA | FnCas12a | 17687:17709 | − | Transgenic |
| | | | | | insert |
| 3J-1 | TTTG | LbCas12a | 17719:17741 | + | 3′ Junction |
| 3J-2 | TATG | LbCas12a-TATV | 17715:17737 | − | 3′ Junction |
| 3F-1 | CTTC | FnCas12a | 17756:17778 | + | 3′ Flanking |
| | | | | | DNA |
| 3F-2 | TTCG | LbCas12a-TYC | 17757:17779 | + | 3′ Flanking |
| | | | | | DNA |
| 3F-3 | TTTG | LbCas12a | 17762:17784 | + | 3′ Flanking |
| | | | | | DNA |
| 3F-4 | GTTA | FnCas12a | 17746:17768 | − | 3′ Flanking |
| | | | | | DNA |
| 3F-5 | TTTA | LbCas12a | 17771:17793 | + | 3′ Flanking |
| | | | | | DNA |
| 3F-6 | TTCA | LbCas12a-TYC | 17752:17774 | − | 3′ Flanking |
| | | | | | DNA |
| 3F-7 | TTTC | LbCas12a | 17753:17775 | − | 3′ Flanking |
| | | | | | DNA |
| 3F-8 | TTCC | LbCas12a-TYC | 17785:17807 | − | 3′ Flanking |
| | | | | | DNA |
| 3F-9 | TTTC | LbCas12a | 17786:17808 | − | 3′ Flanking |
| | | | | | DNA |
| 3F-10 | TTTA | LbCas12a | 17845:17867 | + | 3′ Flanking |
| | | | | | DNA |
| 3F-11 | CTTG | FnCas12a | 17841:17863 | − | 3′ Flanking |
| | | | | | DNA |
| 3F-12 | TTTC | LbCas12a | 17876:17898 | + | 3′ Flanking |
| | | | | | DNA |
| 3F-13 | TTCC | LbCas12a-TYC | 17877:17899 | + | 3′ Flanking |
| | | | | | DNA |
| 3F-14 | TCCA | LbCas12a-TYC | 17878:17900 | + | 3′ Flanking |
| | | | | | DNA |
| 3F-15 | TTCG | LbCas12a-TYC | 17873:17895 | − | 3′ Flanking |
| | | | | | DNA |
| 3F-16 | TTTC | LbCas12a | 17874:17896 | − | 3′ Flanking |
| | | | | | DNA |
| 3F-17 | GTTA | FnCas12a | 17878:17900 | − | 3′ Flanking |
| | | | | | DNA |
| 3F-18 | TTTC | LbCas12a | 17915:17937 | + | 3′ Flanking |
| | | | | | DNA |
| 3F-19 | TTTA | LbCas12a | 17898:17920 | − | 3′ Flanking |
| | | | | | DNA |
| 3F-20 | TTCA | LbCas12a-TYC | 17917:17939 | − | 3′ Flanking |
| | | | | | DNA |
| 3F-21 | TTTC | LbCas12a | 17918:17940 | − | 3′ Flanking |
| | | | | | DNA |
| 3F-22 | TTTA | LbCas12a-TYC | 18017:18039 | + | 3′ Flanking |
| | | | | | DNA |
| 3F-23 | TTTA | LbCas12a-TYC | 18071:18093 | − | 3′ Flanking |
| | | | | | DNA |
| 3F-24 | TTTA | LbCas12a | 18174:18196 | + | 3′ Flanking |
| | | | | | DNA |
| 3F-25 | TTTA | LbCas12a | 18182:18204 | + | 3′ Flanking |
| | | | | | DNA |
| 3F-26 | TATG | LbCas12a-TATV | 18184:18206 | + | 3′ Flanking |
| | | | | | DNA |
| 3F-27 | TTTG | LbCas12a | 18223:18245 | + | 3′ Flanking |
| | | | | | DNA |
| 3F-28 | TTTG | LbCas12a | 18232:18254 | + | 3′ Flanking |
| | | | | | DNA |
| 3F-29 | TTCA | LbCas12a-TYC | 18241:18263 | + | 3′ Flanking |
| | | | | | DNA |
| 3F-30 | TTTG | LbCas12a | 18222:18244 | − | 3′ Flanking |
| | | | | | DNA |
| 3F-31 | TTTG | LbCas12a | 18253:18275 | + | 3′ Flanking |
| | | | | | DNA |
| 3F-32 | TATG | LbCas12a-TATV | 18258:18280 | + | 3′ Flanking |
| | | | | | DNA |
| 3F-33 | TTTA | LbCas12a | 18242:18264 | − | 3′ Flanking |
| | | | | | DNA |
| 3F-34 | TATG | LbCas12a-TATV | 18248:18270 | − | 3′ Flanking |
| | | | | | DNA |
| 3F-35 | TTCA | LbCas12a-TYC | 18273:18295 | + | 3′ Flanking |
| | | | | | DNA |
| 3F-36 | CTTA | FnCas12a | 18253:18275 | − | 3′ Flanking |
| | | | | | DNA |
| 3F-37 | TATA | LbCas12a-TATV | 18276:18298 | − | 3′ Flanking |
| | | | | | DNA |
| 3F-38 | TTTA | LbCas12a | 18278:18300 | − | 3′ Flanking |
| | | | | | DNA |
| 3F-39 | TTTC | LbCas12a | 18314:18336 | + | 3′ Flanking |
| | | | | | DNA |
| 3F-40 | TTCA | LbCas12a-TYC | 18315:18337 | + | 3′ Flanking |
| | | | | | DNA |
| 3F-41 | TTTA | LbCas12a | 18330:18352 | + | 3′ Flanking |
| | | | | | DNA |
| 3F-42 | TATA | LbCas12a-TATV | 18312:18334 | − | 3′ Flanking |
| | | | | | DNA |
| 3F-43 | TATA | LbCas12a-TATV | 18339:18361 | + | 3′ Flanking |
| | | | | | DNA |
| 3F-44 | TATC | LbCas12a-TATV | 18341:18363 | + | 3′ Flanking |
| | | | | | DNA |
| 3F-45 | GTTG | FnCas12a | 18348:18370 | + | 3′ Flanking |
| | | | | | DNA |
| 3F-46 | GTTG | FnCas12a | 18327:18349 | − | 3′ Flanking |
| | | | | | DNA |
| 3F-47 | GTTC | FnCas12a | 18351:18373 | + | 3′ Flanking |
| | | | | | DNA |
| 3F-48 | TTCA | LbCas12a-TYC | 18352:18374 | + | 3′ Flanking |
| | | | | | DNA |
| 3F-49 | TCCG | LbCas12a-TYC | 18330:18352 | − | 3′ Flanking |
| | | | | | DNA |
| 3F-50 | TATC | LbCas12a-TATV | 18332:18354 | − | 3′ Flanking |
| | | | | | DNA |
| 3F-51 | TTCA | LbCas12a-TYC | 18347:18369 | − | 3′ Flanking |
| | | | | | DNA |
| 3F-52 | TTTC | LbCas12a | 18348:18370 | − | 3′ Flanking |
| | | | | | DNA |
| 3F-53 | GTTG | FnCas12a | 18372:18394 | + | 3′ Flanking |
| | | | | | DNA |
| 3F-54 | GTTG | FnCas12a | 18352:18374 | − | 3′ Flanking |
| | | | | | DNA |
| 3F-55 | TTTC | LbCas12a | 18358:18380 | − | 3′ Flanking |
| | | | | | DNA |
| 3F-56 | TTCA | LbCas12a-TYC | 18368:18390 | − | 3′ Flanking |
| | | | | | DNA |
| 3F-57 | GTTC | FnCas12a | 18369:18391 | − | 3′ Flanking |
| | | | | | DNA |
| 3F-58 | GTTG | FnCas12a | 18393:18415 | + | 3′ Flanking |
| | | | | | DNA |
| 3F-59 | TCCA | LbCas12a-TYC | 18377:18399 | − | 3′ Flanking |
| | | | | | DNA |
| 3F-60 | TATG | LbCas12a-TATV | 18386:18408 | − | 3′ Flanking |
| | | | | | DNA |
| 3F-61 | TTTA | LbCas12a | 18388:18410 | − | 3′ Flanking |
| | | | | | DNA |
| 3F-62 | TTTA | LbCas12a-TYC | 18494:18516 | − | 3′ Flanking |
| | | | | | DNA |
| 3F-63 | TTTA | LbCas12a-TYC | 18498:18520 | − | 3′ Flanking |
| | | | | | DNA |
| 3F-64 | TTTC | LbCas12a-TYC | 18575:18597 | + | 3′ Flanking |
| | | | | | DNA |
| 3F-65 | TTTA | LbCas12a | 18617:18639 | + | 3′ Flanking |
| | | | | | DNA |
| 3F-66 | TTTC | LbCas12a | 18645:18667 | + | 3′ Flanking |
| | | | | | DNA |
| 3F-67 | TTCA | LbCas12a-TYC | 18646:18668 | + | 3′ Flanking |
| | | | | | DNA |
| 3F-68 | TTTA | LbCas12a | 18652:18674 | + | 3′ Flanking |
| | | | | | DNA |
| 3F-69 | TCCA | LbCas12a-TYC | 18639:18661 | − | 3′ Flanking |
| | | | | | DNA |
| 3F-70 | TTTC | LbCas12a | 18681:18703 | + | 3′ Flanking |
| | | | | | DNA |
| 3F-71 | TTCA | LbCas12a-TYC | 18682:18704 | + | 3′ Flanking |
| | | | | | DNA |
| 3F-72 | TATC | LbCas12a-TATV | 18682:18704 | − | 3′ Flanking |
| | | | | | DNA |

gRNAs may include a G leader sequence to facilitate transcription start from a pol III promoter positioned 5′ to a gRNA repeat, resulting in GAATTTCTACTAAGTGTAGAT (SEQ ID NO:203) for LbCas12a, or GTAATTTCTACTGTTGTAGAT (SEQ ID NO:204) for FnCas12a and an OgRRS sequence (as shown in the fourth column of Table 27). For Agrobacterium-based plant expression vectors, the cassette further comprises a poly-T transcript termination region (TTTTTTT). These gRNAs can be used to target the Cas12a nuclease to cut within both the OgRRS and CgRRS sequences. Illustrative examples of such gRNAs for FnCas12a expressed in stable plants are shown in Table 28, wherein the gRNA repeat of TAATTTCTACTGTTGTAGAT is underlined, and the poly-T transcript termination sequence of TTTTTTT is shown in italic font.

TABLE 28
Illustrative Examples of gRNAs Useful in
Targeting FnCas12a Nuclease

| gRNA | Sequence |
|---|---|
| gRNA_5F-87 | GTAATTTCTACTGTTGTAGATAAGACTCAT |
| (SEQ ID | TATATTCTAGAAATTTTTTTT |
| NO: 205) | |
| gRNA_3F-45 | GTAATTTCTACTGTTGTAGATTTCAACGGA |
| (SEQ ID | TAATCCTCAATGTTTTTTTTT |
| NO: 206) | |

Any of the OgRRS sequences presented in Table 27 can be used alternatively as a site to insert a CgRRS that is designed using a different OgRRS. For example, a CgRRS can be inserted into a flanking sequence to allow for the excision of the entire transgenic insertion of event Gh_CSM63718. To illustrate this approach, OgRRS 3F-15 is selected as the OgRRS that can be used to design a corresponding CgRRS 3F-45 comprising DNA fragment, and OgRRS 5F-87 is selected as the target site into which the CgRRS 3F-45 comprising DNA fragment is inserted. Using a Cas12a editing system such as with the FnCas12a endonuclease, the OgRRS 5F-87 site is targeted using the gRNA, gRNA_5F-87 presented in Table 28 to cut within the OgRRS 5F-87 site. The CgRRS 3F-45 comprising DNA fragment that comprises the OgRRS 3F-45 target site is then inserted within the cut site that has been introduced into the OgRRS 5F-87 sequence. After selection of a transgenic event comprising the introduced CgRRS 3F-45 site, the event can be bred into another germplasm. When desired, the transgenic insert of Gh_CSM63718 can be excised from the plant using a Cas12a editing system and the gRNA, gRNA_3F-45 as presented in Table 28.

The CgRRS can be introduced into the transgenic insertion locus through multiple methods using a CRISPR system. For example, a CRISPR system can be used for targeting 5′ insertion of a blunt-end double-stranded DNA fragment into a genomic target site of interest such as an OgRRS that is not the OgRRS that has been selected for the design of the CgRRS. The CRISPR-mediated endonuclease activity can introduce a double stand break (DSB) in the selected genomic target site and DNA repair, such as microhomology-driven nonhomologous end-joining DNA repair, resulting in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′-flanking sequence at the cut site of the protospacer in the genomic target site.

The CRISPR system can be introduced into event Gh_CSM63718 by several methods, including but not limited to Agrobacterium-mediated transformation, polyethylene glycol-mediated transformation, biolistic transformation, liposome-mediated transfection, viral transduction, the use of one or more delivery particles, microinjection, or electroporation. One or more expression cassettes encoding the gRNA and/or CRISPR associated protein components of a Type I, Type II, Type III, Type IV, Type V, or Type VI CRISPR-Cas system is transiently introduced into a cell. The CRISPR-associated protein and guide RNA can be synthesized and assembled in vitro to form a ribonucleoprotein complex (RNP). The ribonucleoprotein along with a DNA fragment encoding the CgRRS can be provided to the plant via polyethylene glycol-mediated transformation, biolistic transformation, liposome-mediated transfection, the use of one or more delivery particles, microinjection, or electroporation. The introduced one or more gRNAs, or expression cassettes encoding the gRNA and/or CRISPR associated protein, along with a DNA fragment comprising the CgRRS is provided in sufficient quantity to modify the cell but does not persist after a contemplated period of time has passed or after one or more cell divisions. In such embodiments, no further steps are needed to remove or segregate the one or more expression cassettes encoding the gRNA and/or CRISPR associated protein from the modified cell. Double-stranded DNA fragments can also be transiently introduced into a cell along with one or more expression cassettes encoding the gRNA and/or CRISPR associated protein. The introduced double-stranded DNA fragments are provided in sufficient quantity to modify the cell but do not persist after a contemplated period of time has passed or after one or more cell divisions.

Alternatively, an expression construct comprising one or more expression cassettes for the expression of one or more gRNAs, and an expression construct encoding a Type I, Type II, Type III, Type IV, Type V, or Type VI CRISPR associated protein is stably transformed into event Gh_CSM63718 to modify the plant cell in the targeted region of the transgene insertion locus, to introduce the CgRRS within the desired target locus.

### Example 9: Modification of Cotton Event Gh_CSM63718 with Genome Editing Techniques Using Two Guide RNAs

This example describes how one may alter or excise all or a part of the transgenic insertion present in cotton event Gh_CSM63718, as well as flanking genomic DNA segments, such as by making one or more insertions, deletions, substitutions, or transpositions using genomic editing techniques. Excision of all or a portion of the event Gh_CSM63718 transgenic insertion or expression element(s) or cassette within SEQ ID NO:9 or SEQ ID NO:10 can be performed through genome editing using a variety of methods. In one embodiment, Clustered Regularly Interspersed Short Palindromic Repeats (CRISPR) editing systems comprising a CRISPR associated protein and two cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein, such as but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and two guide RNAs (gRNA) can be introduced into a plant cell comprising the cotton event Gh_CSM63718 to target a specific sequence within the transgene insertion locus. In one embodiment, the CRISPR nuclease system cleaves at two distinct guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination thereof, at the genomic target site.

Sequences corresponding to the 5′ and 3′ flanking genomic sequences, and the transgenic insert of event Gh_CSM63718 (SEQ ID NOs:11, 12, and 9, respectively) and the 5′ and 3′ junction regions (SEQ ID NOs:1-6) were scanned for potential guide RNA recognition sites which comprise a protospacer adjacent motif (PAM) site that will be recognized by a Cas12a endonuclease, operably linked to a guide RNA hybridization site, and the results are shown in Table 27 in Example 8. The identified gRNA recognition sites are located within the 5′ or 3′ flanking genomic sequence, within the 5′ or 3′ junction regions, or within the transgenic insertion.

Two functional guide RNAs (gRNAs) for an RNA guided nuclease system are created to target the event Gh_CSM63718 transgenic insertion locus in a manner that will permit the excision of a fragment of DNA corresponding to either the entire transgenic insertion of event Gh_CSM63718, or a fragment within the transgenic insertion of event Gh_CSM63718 such as the expression cassette(s) or a genetic element within the transgene cassette(s). Illustrative examples are described below using a FnCas12a editing system (see Table 28), in which the gRNAs may include a G leader sequence to facilitate transcription start and a gRNA repeat

| (SEQ ID NO: 204 | |
|---|---|
| | GTAATTTCTACTGTTGTAGAT, |

gRNA repeat underlined), an OgRRS sequence (as shown in the fourth column of Table 27) and a TTTTTTT (poly-T transcript termination region, italic in Table 28) to target the FnCas12a nuclease to the gRNA recognition sites. Similar methods can be used to excise either the entire transgene insertion or a fragment within the transgene insertion such as one or more of the expression cassettes by selecting gRNAs targeted to the specific regions. Alternatively, the LbCas12a editing systems can be used, in which the gRNAs include a G leader sequence to facilitate transcription start and a gRNA repeat

| (SEQ ID NO: 203 | |
|---|---|
| | GAATTTCTACTAAGTGTAGAT, |

gRNA repeat underlined), an OgRRS sequence (as shown in the fourth column of Table 27 above), and a TTTTTTT (poly-T transcript termination region).

To excise the entire transgenic insertion of event Gh_CSM63718, the first gRNA targets an area in the 5′ flanking genomic sequence such as 5F-87 (Table 28), and the second gRNA targets a region in the 3′ flanking genomic sequence such as 3F-45 (Table 28). A transfer DNA (T-DNA) construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding a Cas12a RNA guided nuclease. A second expression cassette comprises a promoter that is operable in a plant cell operably linked to a selection marker gene, such as aadA for conferring resistance to spectinomycin and/or streptomycin. The construct also comprises expression cassettes comprising Polymerase III or Polymerase II promoters operable in a plant cell operably linked to polynucleotides encoding the two gRNAs, gRNA_5F-87 and gRNA_3F-45 (Table 28).

Following Agrobacterium-mediated transformation of cotton comprising event Gh_CSM63718, and upon expression of the integrated polynucleotides, the gRNAs guide the nuclease to each of the two target sites at the transgenic insertion locus, where the nuclease creates a double-stranded break at each target site, resulting in deletion of the region between the target sites, and non-homologous end-joining repair mechanisms joins the flanking regions. Suitable methods known in the art (e.g., PCR, DNA hybridization (Southern) blots, sequencing) are used to identify plants comprising a complete deletion.

### Example 10: Cotton Event Gh_CSM63718 Element-Specific Endpoint Taqman™ Assay and Primers and Probe that Hybridize to the PPO_H_N90 Coding Sequence

This example describes a method useful in identifying or detecting the presence of an element within the inserted T-DNA of event Gh_CSM63718 or a modified event Gh_CSM63718. Illustrative detection methods and materials are provided below.

An element-specific endpoint TaqMan™ thermal amplification method was developed to identify PPO_H_N90 in a sample. The DNA primers and probe used in the endpoint assay for this example are shown in Table 29, although it will be appreciated by those of skill in the art that that other primers and probes may also be used.

TABLE 29
Primers and Probe for Cotton
Gh_CSM63718 Element-Specific Assay

| SEQ ID NO. | Name | Type |
|---|---|---|
| 234 | Primer SQ51334 | PPO_H_N90-specific |
| 235 | Primer SQ51335 | PPO_H_N90-specific |
| 236 | 6FAM ™ probe PB50535 | PPO_H_N90-specific |
| 16 | Primer SQ22496 | Internal control |
| 17 | Primer SQ22497 | Internal control |
| 237 | VIC ™ probe PB13032 | Internal control |

TaqMan thermal amplifications use dual-labeled Taqman MGB (Minor Groove Binder) probes incorporating 5′ fluorescent reporter dye 6-FAM™ or VIC™ and a 3′ nonfluorescent quencher. For TaqMan MGB (Minor Groove Binder) probes, the 5′ exonuclease activity of Taq DNA polymerase cleaves the probe from the 5′-end, between the fluorophore and quencher. When hybridized to the target DNA strand, quencher and fluorophore are separated enough to produce a fluorescent signal, thus releasing fluorescence. The pair of primers when used with these reaction methods and the probe produce a DNA amplicon that is diagnostic for cotton event Gh_CSM63718 element PPO_H_N90. The controls for this analysis should include a positive control containing cotton event Gh_CSM63718 element PPO_H_N90, a negative control from a non-transgenic plant or a transgenic plant that does not contain cotton event Gh_CSM63718, and a negative control that contains no template DNA. Additionally, a control for the PCR reaction should optimally include internal control primers and an internal control probe specific to a single copy gene in the cotton genome. These assays are optimized for use with the Applied Biosystems GeneAmp® PCR System 9700 (Thermo Fisher Scientific) run at maximum speed, but other equipment may be used.

Examples of PCR reaction components and cycling conditions useful for the PPO_H N90 element-specific qualitative endpoint TaqMan PCR assay for cotton event Gh_CSM63718 are presented in Table 30 and Table 31. The extracted DNA template was a leaf DNA sample, a negative control (non-transgenic cotton DNA), no template (water) control, or a positive control containing cotton event Gh_CSM63718 DNA.

TABLE 30
Gh_CSM63718 Element-Specific Endpoint TaqMan ™ PCR
Reaction Components

| | Stock | | |
|---|---|---|---|
| | Concen- | | Final |
| | tration | Volume | Concen- |
| Reagent | (□M) | (□l) | tration |
| Reaction Volume | | 5 | |
| Master Mix | | 2.40 | 1 X |
| PPO_H_N90 Specific Primer | 100 | 0.02 | 0.4 □M |
| SQ51334 (SEQ ID NO: 234) | | | |
| PPO_H_N90 Specific Primer | 100 | 0.02 | 0.4 □M |
| SQ51335 (SEQ ID NO: 235) | | | |
| PPO_H_N90 Specific 6FAM ™ probe | 100 | 0.01 | 0.2 □M |
| PB50535 (SEQ ID NO: 236) | | | |
| Internal control primer | 100 | 0.02 | 0.4 □M |
| SQ22496 (SEQ ID NO: 16) | | | |
| Internal control primer | 100 | 0.02 | 0.4 □M |
| SQ22497 (SEQ ID NO: 17) | | | |
| Internal control VIC ™ probe | 100 | 0.01 | 0.2 □M |
| PB13032 (SEQ ID NO: 237) | | | |
| Extracted DNA template | | 2.5 | |

TABLE 31
Endpoint TaqMan ™ Thermocycler Conditions

| Step No. | Cycle No. | Settings |
|---|---|---|
| 1 | 1 | 95° C., 20 seconds |
| 2 | 40 | 95° C., 3 seconds |
| | | 60° C., 20 seconds |

### REFERENCES

G3. V. den Broeck. M. P. Timko, A. P. Kausch, AR. Cashmore, MLV. Montagu and L. Herrera-Estrella (185), Targeting of a foreign protein to chloroplasts by fusion to the transit peptide from the small subunit of ribulose 1,5-bisphosphate carboxylase. Nature 313:358-63.
X. Chen, L. Levine and P.-Y. Kwok (1999). Fluorescence polarization in homogeneous nucleic acid analysis. Genome Res. 9:492-498.
S. Cheng, C. Fockler, W. M. Barnes and R. Higuchi (1994). Effective amplification of long targets from cloned inserts and human genomic DNA. Proc. Natl. Acad. Sci. USA 91:5695-5699.
C. D. Day, E. Lee, J. Kobayashi, L. D. Holappa, H. Albert and D. W. Ow (2000). Transgene integration into the same chromosome location can produce alleles that express at a predictable level, or alleles that are differentially silenced. Genes Dev. 14 (22):2869-2880.
F. B. Dean, S. Hosono, L. Fang, X. Wu, A. F. Faruqi, P. Bray-Ward, Z. Sun, Q. Zong, Y. Du, J. Du, M. Driscoll, W. Song, S. F. Kingsmore, M. Egholm and R. S. Lasken (2002). Proc. Natd. Acad Sci. USA 99:5261-5266.
A. D. Ellington and J. W. Szostak, J. (1990). In vitro selection of RNA molecules that bind specific ligands. Nature 346:818-822.
R. D. Finn, A. Bateman, J. Clements, P. Coggill, R. Y. Eberhardt, S. R. Eddy, A. Heger, K. Ketherington, L. HolIm, J. Mistry, E. L. L. Sonnhmmer, J. Tate and M. Punta (2014). Pfam: the protein families database. Nucleic Acids Research 42 (D1): D222-D230.
A. Fire and S. Q. Xu (1995). Rolling replication of short DNA circles. Proc. Natl. Acad Sci. USA 92:4641-4645.
R. T. Fraley, S. G. Roger, R, B. Horsch, P. R. Sanders, J. S. Flick, S. P. Adams, M. L. Bittner, L. A. Brand, C. L. Fink, J. S. Fry, G. R. Galluppi, S. B. Goldberg, N. L. loffmann and S. C. Woo (1983). Expression of bacterial genes in plant cells. Proc Natl Acad Sci U.S.A., 80 (15): 4803-07.
L. Gao, D. B. T. Cox, W. X. Yan, J. C. Manteiga, M. W. Schneider, T. Yamano, H. Nishimasu, O. Nureki, N. Crosetto and F. Zhang (2017). Engineered Cpf1 variants with altered PAM specificities. Nature Biotechnology 35(8):789-792.
J. C. Guatelli, K. M. Whitefield, D. Y. Kwon, K. J. Barringer, D. D. Richman and T. R. Gingeras (1990). Isothermal, in vitro amplification of nucleic acids by a multienzyme reaction modeled after retroviral replication. Proc. Natl. Acad. Sci. USA 87:1874-1878.
I. M. Heap (2021). International Survey of Herbicide Resistant Weeds. http://www.weedscience.org/Home. aspx.
D. Hyatt, G.-L. Chen, P. F. LoCascio, M. L. Land, F. W. Larimer and L. J. Hauser (2010). Prodigal: prokaryotic gene recognition and translation initiation site identification. BMC Bioinformatics 11, 119.
S. Khan, A. Latif, S. Q, Ahmad, F. Ahmad and M. Ghaffar (2010). Double haploid technique: in cotton and other species. International Journal of Applied Agricultural Research 5:649-655.
C. T. Larue, J. E. Ream, X. Zhou, F. Moshiri, A. Howe, M. Goley, O. C. Sparks, S. T. Voss, E. Hall, C. Ellis, J. Weihe, Q. Qi, D. Ribeiro, X. Wei, S. Guo, A. G. Evdokimov, M. J. Varagona and J. K. Roberts (2019). Microbial HemG-type protoporphyrinogen IX oxidase enzymes for biotechnology applications in plant herbicide tolerance traits. Pest Management Sci. 76 (3):1031-1038.
I. Lermontova, E. Kruse, H. P. Mock, and B. Grimm (1997). Cloning and characterization of plastidal and mitochondrial isoform of tobacco protoporphyrinogen IX oxidase. Proc. Natl. Acad. Sci. USA 94:8895-8900.
Q. Li, Y. Huo, Y. Guo, X. Zheng, W. Sun and Z. Hao (2017) Generation and applications of a DNA aptamer against gremlin-1. Molecules 22:706
D. Liu, S. L. Daubendiek, M. Zillman, K. Ryan and E. T. Kool (1996). Rolling circle DNA synthesis: small circular oligonucleotides as efficient templates for DNA polymerases. J. Am. Chem. Soc. 118:1587-1594.
L. Long, Y.-M. Feng, S.-Z. Shang, J. R. Zhao, G.-Y. Hu, F.-C. Xu, C.-P. Song, S.-X. Jin and W. Gao (2023). In vivo maternal haploid induction system in cotton. Plant Physiology 00:1-4.
P. M. Lizardi, X. Huang, Z. Zhu, P. Bray-Ward, D. C. Thomas and D. C. Ward (1998). Mutation detection and single-molecule counting using isothermal rolling-circle amplification. Nature Genetics 19:225-232.
T. T. Nikiforov, R. B. Rendie, P. Goelet, Y.-H. Rogers, M. L. Kotewicz, S. Anderson, G. L. Trainor and M. R. Knapp (1994). Genetic bit analysis: a solid phase method for typing single nucleotide polymorphisms. Nucleic Acids Res. 22:4167-4175.
T. Notomi, H. Okayama, H. Masubuchi, T. Yonekawa, K. Watanabe, N. Amino, T. Hase (2000). Loop-mediated isothermal amplification of DNA”. Nucleic Acids Res. 28 (12): 63e-63.
M. D. Pearson, L. Nguyen, Y. Zhao, W. L. McKenna, T. J. Morin and W. B. Dunbar (2019). Past and accurate quantification of insertion-site specific transgene levels from raw seed samples using solid--state nanopore technology. PloS One 14 (12): e0226719.
D. L. Robertson and J. G. Joyce (1990). Selection in vitro of an RNA enzyme that specifically cleaves single-stranded DNA. Nature 344:467-468.
S. S. Sekhon, H. J. Un, W. R. Shin, SH. Lee, J. Min, J. Y. Ahn and Y. H. Kim. (2017). Aptabody-aptatope interactions in aptablotting assays. Nanoscale 9:7464-7475.
D. Shoup, S. Duncan and IA. Ciampitti (2016). Cotton Production Handbook. Kansas State University Agricultural Experiment Station and Cooperative Extension Service.
M. Svobodova, V. Skouridou, M. Jauset-Rubio, 1. Viéitez, A. Fernández-Villar. J. J. C. Alvargonzalez, E. Poveda, C. B. Bofill, T. Sans, A. Bashammakh, A. O. Alyoubi and C. K. O'Sullivan (2021). Aptamer sandwich assay for detection of SARS-CoV-2-spike protein antigen. ACS Omega 6:35657-35666.
A. D. Taylor, L. Mataseje, C. J. Urfano, L. Schmidt, K. S. Antonation, M. R. Mulvey and C. R. Corbett (2018). Evaluation of Oxford Nanopore's MinION Sequencing Device for Microbial Whole Genome Sequencing Applications. Scientific Reports 8:10931.
C. Tuerk and L. Gold (1990). Systematic evolution of ligands by exponential enrichment: RNA ligands to bacteriophage T4 DNA polymerase. Science 249:505--510.
S. Tyagi and F. R. Kramer (1996). Molecular beacons: probes that fluoresce upon hybridization. Nature Biotechnology 14:303-308.
A. Verkest, S. Bourout, J. Debaveye, K. Reynaert, B. Saey, I. Van den Brande and K. D′Halluin (2019). Impact of differential DNA methylation on transgene expression in cotton (Gossypium hirsutum L.) events generated by targeted sequence insertion. Plant Biotechnology J. 17:1236-1247.
M. Vincent, Y. Xu and H. Kong (2004). Helicase-dependent isothermal DNA amplification. EMBO Reports 5(8):795-800.
Y. Wan, J. F. Petolino and J. M. Widholm (1989). Efficient Production of Doubled Haploid Plants Through Colchicine Treatment of Anther-Derived Maize Callus. Theor. Appl. Genet. 77:889-892.
C. Wang, K. C. Glenn, C. Kessenich, E. Bell, L. A. Burzio, M. S. Koch, B. Li and A. Silvanovich (2016). Safety assessment of dicamba mono-oxygenases that confer dicamba tolerance to various crops. Regulatory Toxicology and Pharmacology 81:171-182.
T. Wang, C. Chen, L. M. Larcher, R. A. Barrero and R. N. Veedu (2019). Three decades of nucleic acid aptamer technologies: Lessons learned, progress and opportunities on aptarner development. Biotechnol. A dv. 37:28-50.
Y. Wang, Y. Zhao, A. Bollas, Y. Wang and K. F. Au (2021). Nanopore sequencing technology, bioinformatics and applications. Nature Biotechnology 39:1348-1365.
Y. Wang, Z. Li and H. Yu (2020). Aptamer-based Western blot for selective protein recognition. Frontiers in Chem. 8:1-9.
W. R. Widner and H. R. Whiteley (1990). Location of the Dipteran Specificity Region in a Lepidopteran-Dipteran Crystal Protein from Bacillus thuringiensis. J. Bacteriol. 172:2826-32.
M. Winge (2000). Pyrosequencing-a new approach to DNA analysis. Innovation in Pharma. Tech. 00:18-24.
Y. Xu, X. Zhou and W. Zhang (2008) MicroRNA prediction with a novel ranking algorithm based on random walk. Bioinformatics 24 (13):i50-i58.
S. Yang, J. Fresnedo-Ramirez, M. Wang, L. Cote, P. Schweitzer, P. Barba, E. M. Takacs, M. Clark, J. Luby. D. C. Manns, G. Sacks, A. K. Mansfield, J. Londo, A. Fennell, D. Gadoury, B. Reisch, L. Cadle-Davidson, and Q. Sun (2016). A next-generation marker genotyping platform (AmpSeq) in heterozygous crops: a case study for marker-assisted selection in grapevine. Hort. Res. 3:16002. B. Zetsche, J. S. Gootenberg, 0.0. Abudayyeh, I. M. Slaymaker, K. S. Makarova, P. Essletzbichler, S. E.
Volz, J. Joung, J. v.d. Oost, A. Regev, E. V. Koonin and F. Zhang (2015). Cpf1 is a single RNA-guided endonuclease of a class 2 CRISPR-Cas system. Cell 163(3):759-771.

1. A recombinant DNA molecule comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing.

2. The recombinant DNA molecule of claim 1, wherein:
a) the recombinant DNA molecule is derived from a cotton plant, seed, plant part, plant cell, progeny plant, or commodity product comprising cotton event Gh_CSM63718, a representative sample of seed comprising the event having been deposited as ATCC Accession No. PTA-127638;
b) the recombinant DNA molecule is comprised in a cotton plant, seed, plant part, plant cell, or progeny plant comprising cotton event Gh_CSM63718, or a commodity product produced therefrom, a representative sample of seed comprising the event having been deposited as ATCC Accession No. PTA-127638;
c) the recombinant DNA molecule is formed by the insertion of a heterologous nucleic acid molecule into the genomic DNA of a cotton plant or cotton cell; or
d) the recombinant DNA molecule comprises an amplicon diagnostic for the presence of cotton event Gh_CSM63718.

3-5. (canceled)

6. A DNA molecule comprising a polynucleotide segment of sufficient length to function as a DNA probe, wherein said DNA molecule:
a) hybridizes specifically under stringent hybridization conditions with the recombinant DNA molecule of claim 2, wherein the recombinant DNA molecule is derived from a cotton plant, seed, plant part, plant cell, progeny plant, or commodity product comprising cotton event Gh_CSM63718 DNA in a sample, wherein detecting hybridization of the DNA molecule under the stringent hybridization conditions is diagnostic for the presence of cotton event Gh_CSM63718 in the sample; or
b) is specific for detecting in a sample at least one of:
a 5′ junction sequence between flanking cotton genomic DNA and the transgenic insert of cotton event Gh_CSM63718;
a 3′ junction sequence between the transgenic insert of cotton event Gh_CSM63718 and flanking cotton genomic DNA;
SEQ ID NO:9; or
a fragment of SEQ ID NO:9 comprising a sufficient length of contiguous nucleotides of SEQ ID NO:9 to identify the sequence as a fragment of the transgenic insert of Gh_CSM63718.

7. (canceled)

8. The DNA molecule of claim 6, wherein:
a) the DNA molecule comprises SEQ ID NO:21;
b) the DNA molecule comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing; or
c) the sample is derived from a cotton plant, seed, plant part, plant cell, progeny plant, or commodity product.

9. (canceled)

10. A pair of DNA molecules comprising a first DNA molecule and a second DNA molecule, wherein the first and the second DNA molecules are different from one another, and each comprise a fragment of SEQ ID NO:10 or a complement thereof and function as DNA primers when used together in an amplification reaction with DNA comprising the recombinant DNA molecule of claim 2, wherein the recombinant DNA molecule is derived from a cotton plant, seed, plant part, plant cell, progeny plant, or commodity product comprising cotton event Gh_CSM63718 to produce an amplicon diagnostic for cotton event Gh_CSM63718 in a sample.

11. The pair of DNA molecules of claim 10, wherein;
a) the first and the second DNA molecules comprise SEQ ID NO:19 and SEQ ID NO:20;
b) the amplicon comprises a nucleotide sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:9, SEQ ID NO:10, and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 17,736-17,737 of SEQ ID NO:10; or
d) the sample is derived from a cotton plant, seed, plant part, plant cell, progeny plant, or commodity product.

12-14. (canceled)

15. A method of detecting the presence of cotton event Gh_CSM63718 in a sample derived from a cotton seed, plant, plant part, plant cell, progeny plant, or commodity product, the method comprising:
a) contacting the sample with the DNA molecule that functions as a DNA probe of claim 6; subjecting the sample and the DNA molecule that functions as a probe to stringent hybridization conditions; and detecting the hybridization of the DNA molecule that functions as a probe to a DNA molecule in the sample, wherein the hybridization of the DNA molecule that functions as a probe to the DNA molecule in the sample is diagnostic for the presence of cotton event Gh_CSM63718 in the sample; or
b) contacting the sample with said DNA molecule that functions as said DNA probe; and performing a sequencing reaction to produce a target sequence, wherein the target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 17,736-17,737 of SEQ ID NO:10.

16. A method of detecting the presence of cotton event Gh_CSM63718 in a sample derived from a cotton seed, plant, plant part or plant cell, progeny plant or commodity product, the method comprising:
a) contacting the sample with the pair of DNA molecules of claim 10;
b) performing an amplification reaction sufficient to produce a DNA amplicon; and
c) detecting the presence of the DNA amplicon;
wherein the DNA amplicon comprises at least one of:
a 5′ junction sequence between flanking cotton genomic DNA and the transgenic insert of cotton event Gh_CSM63718,
a 3′ junction sequence between flanking cotton genomic DNA and the transgenic insert of cotton event Gh_CSM63718,
SEQ ID NO: 9, and
a fragment of SEQ ID NO: 9 comprising a sufficient length of contiguous nucleotides of SEQ ID NO: 9 to identify the sequence as a fragment of the transgenic insert of Gh_CSM63718; and
wherein the presence of the DNA amplicon indicates the presence of cotton event Gh_CSM63718 in the sample.

17. The method of claim 16, wherein:
a) the DNA amplicon is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length; or
b) the DNA amplicon comprises a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 17,736-17,737 of SEQ ID NO:10.

18-19. (canceled)

20. A method of detecting the presence of cotton event Gh_CSM63718 in a sample derived from a cotton seed, plant, plant part, cell, progeny plant or commodity product comprising the recombinant DNA molecule of claim 2, wherein the recombinant DNA molecule is derived from a cotton plant, seed, plant part, plant cell, progeny plant, or commodity product comprising cotton event Gh_CSM63718, the method comprising:
a) contacting the sample with an antibody specific for the PPO protein encoded by cotton event Gh_CSM63718, an antibody specific for the TDO protein encoded by cotton event Gh_CSM63718, or a combination thereof; and
b) detecting binding of the antibody or antibodies to the protein or proteins in the sample;
wherein the binding of the antibody or antibodies indicates the presence of cotton event Gh_CSM63718 in the sample.

21. The method of claim 20, wherein the method further comprises:
a) contacting the sample with an antibody specific for the DMO protein encoded by cotton event Gh_CSM63718, an antibody specific for the EPSPS protein encoded by cotton event Gh_CSM63718, an antibody specific for the PAT protein encoded by cotton event Gh_CSM63718, or a combination of any thereof; and
b) detecting binding of the antibody or antibodies to the protein or proteins in the sample;
wherein the binding of the antibody or antibodies indicates the presence of cotton event Gh_CSM63718 in the sample.

22. A DNA detection kit or a protein detection kit for detecting the presence of cotton event Gh_CSM63718 in a sample,
wherein the DNA detection kit comprises:
a) a pair of DNA molecules comprising a first DNA molecule and a second DNA molecule, wherein the first and the second DNA molecules are different from one another, and each comprise a fragment of SEQ ID NO:10 or a complement thereof and function as DNA primers when used together in an amplification reaction with DNA comprising cotton event Gh_CSM63718 to produce an amplicon diagnostic for cotton event Gh_CSM63718 in a sample; or
b) the DNA molecule that functions as a probe of claim 6; or
wherein the protein detection kit comprises:
an antibody specific for the PPO protein encoded by cotton event Gh_CSM63718, an antibody specific for the TDO protein encoded by cotton event Gh_CSM63718, or a combination thereof; wherein detecting binding of the antibody or antibodies to the protein(s) encoded by cotton event Gh_CSM63718 in a sample is diagnostic for the presence of cotton event Gh_CSM63718 in the sample.

23. (canceled)

24. The DNA detection kit or the protein detection kit of claim 22, wherein the protein detection kit further comprises an antibody specific for the DMO protein encoded by cotton event Gh_CSM63718, an antibody specific for the EPSPS protein encoded by cotton event Gh_CSM63718, an antibody specific for the PAT protein encoded by cotton event Gh_CSM63718, or a combination of any thereof.

25. A method of determining the zygosity of a cotton plant, plant part, plant seed, or plant cell comprising the recombinant DNA molecule of claim 2, wherein the recombinant DNA molecule is derived from a cotton plant, seed, plant part, plant cell, progeny plant, or commodity product comprising cotton event Gh_CSM63718, the method comprising:
a) contacting a sample comprising DNA derived from the cotton plant, plant part, plant seed, or plant cell with a first primer set capable of producing a first amplicon diagnostic for the presence of cotton event Gh_CSM63718, and a second primer set capable of producing a second amplicon diagnostic for wildtype cotton genomic DNA not comprising cotton event Gh_CSM63718; performing a nucleic acid amplification reaction; and detecting the first amplicon and the second amplicon, wherein the presence of both amplicons indicates that the plant, plant part, seed or cell is heterozygous for cotton event Gh_CSM63718, and the presence of only the first amplicon indicates that the plant, plant part, seed, or cell is homozygous for cotton event Gh_CSM63718; or
b) contacting a sample comprising DNA derived from the cotton plant, plant part, plant seed, or plant cell with a probe set comprising at least a first probe that specifically hybridizes to cotton event Gh_CSM63718, and at least a second probe that specifically hybridizes to cotton genomic DNA that was disrupted by insertion of the heterologous DNA of cotton event Gh_CSM63718 but does not hybridize to cotton event Gh_CSM63718; and hybridizing the probe set with the sample under stringent hybridization conditions, wherein detecting hybridization of only the first probe under the hybridization conditions is diagnostic for a cotton plant, plant part, seed or plant cell homozygous for cotton event Gh_CSM63718, and wherein detecting hybridization of both the first probe and the second probe under the hybridization conditions is diagnostic for a cotton plant, plant part, seed, or plant cell heterozygous for cotton event Gh_CSM63718.

26. The method of claim 25, wherein:
a) the first primer set comprises SEQ ID NO:19 and SEQ ID NO:20, and the second primer set comprises SEQ ID NO:19 and SEQ ID NO:22; or
b) the probe set comprises SEQ ID NO:21 and SEQ ID NO:23.

27-28. (canceled)

29. A DNA construct comprising:
a) a first expression cassette, a second expression cassette, a third expression cassette, a fourth expression cassette, and a fifth expression cassette, wherein:
i) the first expression cassette comprises in operable linkage i) a ribulose bisphosphate carboxylase/oxygenase (RuBisCO) activase gene promoter, and a leader sequence from Arabidopsis thaliana, ii) a codon-optimized phosphinothricin N-acetyltransferase (PAT) coding sequence from Streptomyces viridochromogenes, and iii) a 3′ UTR of a small heat shock protein (Hsp20) from Medicago truncatula;
ii) the second expression cassette comprises in operable linkage i) an enhancer from the strawberry vein banding virus (SVBV) fused to the promoter and 5′ UTR from a CAB1 (Chlorophyll A/B Binding Protein) gene from Cucumis melo, ii) a codon-optimized triketone dioxygenase (TDO) coding sequence from Oryza sativa, and iii) a 3′ UTR of a TMA7 (translation machinery associated 7) protein from Medicago truncatula;
iii) the third expression cassette comprises in operable linkage i) a polyubiquitin gene (UBQ10) promoter, a leader and an intron sequence from Arabidopsis thaliana, ii) an N-terminal chloroplast transit peptide coding sequence of APG6 (Albino and Pale Green 6) from Arabidopsis thaliana fused to a codon-optimized dicamba monooxygenase (DMO) coding sequence from Stenotrophomonas maltophilia; and iii) a 3′ UTR of an aluminum-induced Sali3-2 protein from Medicago truncatula;
iv) the fourth expression cassette comprises in operable linkage i) an enhancer of the 35S gene from Figwort Mosaic Virus (FMV), ii) a promoter, a leader sequence, and an intron sequence of the elongation factor 1A gene (ELF1a) from Arabidopsis thaliana, iii) an N-terminal chloroplast transit peptide of granule bound starch synthase I from Triticum aestivum fused to a codon optimized 5-enolpyruvylshikimate-3-phosphate synthase gene (EPSPS) from Agrobacterium sp strain CP4, and iv) a 3′ UTR of a ribulose 1,5-bisphosphate carboxylase small subunit E9 (rbcS-E9) gene from Pisum sativum; and
v) the fifth expression cassette comprises in operable linkage i) an enhancer derived from multiple enhancer sequences from Arabidopsis thaliana, ii) a promoter sequence designed from multiple promoter sequences from Arabidopsis thaliana, iii) an intron and 5′ UTR for a cytochrome C oxidase subunit VIa gene from Arabidopsis thaliana fused a 5′ UTR designed from multiple 5′ UTR sequences from Arabidopsis thaliana, iv) an N-terminal chloroplast transit peptide coding sequence of APG6 (Albino and Pale Green 6) from Arabidopsis thaliana, with monocot codon usage, fused to the coding region of a protoporphyrinogen oxidase (PPO) gene from Enterobacter cloacae with codons optimized for cotton, and v) a 3′ UTR from the fiber FbLate-2 gene from Gossypium barbadense; or
b) a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the full length of SEQ ID NO: 9; and wherein the DNA construct comprises at the 5′ and/or 3′ end of said construct (i) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15.

30. The DNA construct of claim 29, wherein the DNA construct comprises the first expression cassette, the second expression cassette, the third expression cassette, the fourth expression cassette, and the fifth expression cassette, and wherein:
a) the DNA construct comprises SEQ ID NO:9, or
b) the DNA construct further comprises at the 5′ and/or 3′ end of said construct:
i) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO: 14; or
ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or SEQ ID NO:15.

31-32. (canceled)

33. The DNA construct of claim 29, wherein:
a) the construct comprises at the 5′ end of said construct one or more nucleotide sequences selected from SEQ ID NOs:58-127; or
b) the construct comprises at the 3′ end of said construct one or more nucleotide sequences selected from SEQ ID NOs:128-197.

34. A cotton plant, plant part, plant seed, or plant cell that comprises cotton event Gh_CSM63718, a representative sample of seed comprising cotton event Gh_CSM63718 having been deposited under ATCC Accession No. PTA-127638.

35. A method for controlling or preventing weed growth in an area, the method comprising planting the cotton plant or plant seed of claim 34 in the area and applying an effective amount of at least one herbicide selected from the group consisting of glufosinate, a ß-triketone HPPD inhibitor, dicamba, glyphosate, a PPO herbicide, and any combination thereof, to control weeds in the area without injury to the cotton or with less than about 10% injury to the cotton.

36. The method of claim 35, wherein:
a) applying the effective amount of at least one herbicide comprises applying at least two or more herbicides selected from the group consisting of glufosinate, a ß-triketone HPPD inhibitor, dicamba, glypohsate, a PPO herbicide, and any combination thereof over a growing season; or
b) the effective amount of glufosinate is about 0.4 lb/acre to about 1.6 lb/acre over a growing season; wherein the ß-triketone HPPD inhibitor comprises mesotrione and the effective amount of mesotrione is about 0.09 lb/acre to about 0.36 lb/acre; wherein the effective amount of dicamba is about 0.5 lb/acre to about 2 lb/acre over a growing season; wherein the effective amount of glyphosate is about 0.5 lb/acre to about 2.5 lb/acre over a growing season; and wherein the effective amount of the PPO herbicide is about 0.0009 lb/acre to about 1.5 lb/acre over a growing season.

37. A method for controlling volunteer cotton comprising the cotton plant or the plant seed of claim 34 in an area, the method comprising applying an herbicidally effective amount of at least one herbicide other than glufosinate, a ß-triketone HPPD inhibitor, dicamba, glyphosate, or a PPO herbicide, wherein the herbicide application prevents growth of cotton comprising cotton event Gh_CSM63718.

38. The method of claim 37 wherein the herbicide other than glufosinate, a ß-triketone HPPD inhibitor, dicamba, glyphosate, or a PPO herbicide is selected from the group consisting of atrazine, topramezone, clopyralid, pyrithiobac, fluometuron, (3-(3,4-dichlorophenyl)-1,1-dimethylurea) (DCMU), 2,4-D, thidiazuron, dichlorprop-p 2-ethylhexyl ester, dichlorprop-p, trifloxysulfuron, paraquat, diquat, and combinations of any thereof.

39. A method of obtaining a seed of a cotton plant or a cotton plant that is tolerant to glufosinate, a ß-triketone HPPD inhibitor, dicamba, glyphosate, a PPO herbicide, or any combination thereof, the method comprising:
a) obtaining a population of progeny seed or plants grown therefrom of the cotton plant of claim 34, at least one of which comprises cotton event Gh_CSM63718; and
b) identifying at least a first progeny seed or plant grown therefrom that comprises cotton event Gh_CSM63718.

40. The method of claim 39, wherein identifying the progeny seed or plant grown therefrom that comprises cotton event Gh_CSM63718 comprises:
a) growing the progeny seed or plant to produce progeny plants, treating the progeny plants with an effective amount of at least one herbicide selected from the group consisting of glufosinate, a ß-triketone HPPD inhibitor, dicamba, glyphosate, a PPO herbicide, and combinations of any thereof, and selecting a progeny plant that is tolerant to the at least one herbicide selected from the group consisting of glufosinate, a ß-triketone HPPD inhibitor, dicamba, glyphosate, a PPO herbicide, and combinations of any thereof;
b) detecting the presence of cotton event Gh_CSM63718 in a sample derived from the progeny seed or plant grown therefrom; or
c) detecting the presence of at least one protein encoded by cotton event Gh_CSM63718 in a sample derived from the progeny seed or plant grown therefrom.

41-42. (canceled)

43. A method of improving tolerance to at least one herbicide selected from the group consisting of glufosinate, a ß-triketone HPPD inhibitor, dicamba, glyphosate, a PPO herbicide, and combinations of any thereof in a cotton plant comprising:
a) inserting the DNA construct of claim 29 into the genome of a cotton cell;
b) generating a cotton plant from the cotton cell; and
c) selecting a cotton plant comprising the DNA construct.

44. The method of claim 43, wherein:
a) the selecting comprises treating the cotton cell or plant with an effective amount of at least one herbicide selected from the group consisting of glufosinate, a ß-triketone HPPD inhibitor, dicamba, glyphosate, a PPO herbicide, and combinations of any thereof; or
b) the selecting comprises treating the cotton cell or plant with an effective amount of at least one herbicide selected from the group consisting of glufosinate, a ß-triketone HPPD inhibitor, dicamba, glyphosate, a PPO herbicide, and combinations of any thereof, and wherein the effective amount of glufosinate is about 0.4 lb/acre to about 1.6 lb/acre over a growing season; wherein the ß-triketone HPPD inhibitor comprises mesotrione and the effective amount of mesotrione is about 0.09 lb/acre to about 0.36 lb/acre; wherein the effective amount of dicamba is about 0.5 lb/acre to about 2 lb/acre over a growing season; wherein the effective amount of glyphosate is about 0.5 lb/acre to about 2.5 lb/acre over a growing season; and wherein the effective amount of the PPO herbicide is about 0.0009 lb/acre to about 1.5 lb/acre over a growing season.

45. A cotton plant, plant seed, plant part, or plant cell comprising the recombinant DNA molecule of claim 1.

46. The cotton plant, plant seed, plant part, or plant cell of claim 45, wherein:
a) the plant, plant seed, plant part, or plant cell expresses at least one herbicide tolerance gene selected from the group consisting of phosphinothricin N-acetyltransferase (PAT), triketone dioxygenase (TDO), dicamba monooxygenase (DMO), 5-enolpyruvylshikimate-3-phosphate synthase (EPSPS), protoporphyrinogen oxidase (PPO), and any combination thereof;
b) the plant, plant seed, plant part, or plant cell is tolerant to at least one herbicide selected from the group consisting of glufosinate, ß-triketone HPPD inhibitors, dicamba, glyphosate, PPO herbicides, and combinations of any thereof;
c) the plant, plant seed, plant part, or plant cell comprises cotton event Gh_CSM63718, a representative sample of seed comprising the event having been deposited under ATCC Accession No. PTA-127638;
d) the plant, plant seed, plant part, or plant cell is further defined as a progeny plant of any generation of a cotton plant comprising cotton event Gh_CSM63718, or a cotton plant part, plant seed, or plant cell derived therefrom;
e) the plant part comprises a microspore, pollen, an anther, an ovule, an ovary, a boll, a flower, an embryo, a stem, a bud, a node, a leaf, a root, or a callus; or
f) the plant, plant seed, plant part, or plant cell is tolerant to at least one herbicide selected from the group consisting of glufosinate, ß-triketone HPPD inhibitors, dicamba, glyphosate, PPO herbicides, and combinations of any thereof, and wherein the ß-triketone HPPD inhibitor is selected from the group consisting of mesotrione, benzobicyclon (BBC), tembotrione, sulcotrione, tefuryltrione, and combinations of any thereof, and wherein the PPO herbicide is selected from the group consisting of diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof.

47-50. (canceled)

51. The cotton plant, plant part, plant seed, or plant cell of claim 34, wherein the plant part comprises a microspore, pollen, an anther, an ovule, an ovary, a boll, a flower, an embryo, a stem, a bud, a node, a leaf, a root, or a callus.

52. A cotton plant, plant seed, plant part, or plant cell obtained by the method of claim 39.

53. A cotton plant, plant seed, plant part, or plant cell obtained by the method of claim 3.

54. A cotton plant, plant cell, plant part, or plant seed comprising a recombinant DNA construct integrated in chromosome 21, wherein the recombinant DNA construct confers tolerance to at least one herbicide selected from the group consisting of glufosinate, a ß-triketone HPPD inhibitor, dicamba, glyphosate, a PPO herbicide, and combinations of any thereof, and wherein the recombinant DNA construct is integrated in a position of said chromosome flanked by at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15.

55. The cotton plant, plant cell, plant part, or plant seed of claim 54, wherein:
a) the at least 50 contiguous nucleotides of SEQ ID NO:11 or SEQ ID NO:14 comprise one or more nucleotide sequences selected from SEQ ID NOs:58-127; or
b) the at least 50 contiguous nucleotides of SEQ ID NO:12 or SEQ ID NO:15 comprise one or more nucleotide sequences selected from SEQ ID NOs:128-197.

56-57. (canceled)

58. The cotton plant, plant cell, plant part, or plant seed of claim 46, wherein the diphenylether is selected from the group consisting of acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof; the N-phenylphthalimide is selected from the group consisting of cinidon-ethyl, flumiclorac, flumiclorac-pentyl, and flumioxazin; the oxadiazole is selected from the group consisting of oxadiargyl and oxadiazon; the oxazolidinedione is pentoxazone; the phenylpyrazole is selected from the group consisting of fluazolate, pyraflufen, and pyraflufen-ethyl; the pyrimidinedione is selected from the group consisting of benzfendizone, butafenacil, epyrifencacil (5-3100), flupropacil, flufenoximacil, saflufenacil, and tiafenacil; the thiadiazole is selected from the group consisting of fluthiacet-methyl and thidiazimin; the triazolinone is selected from the group consisting of azafenidin, bencarbazone, carfentrazone, its salts and esters, and sulfentrazone; the benzoxazinone derivative is 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)); or the other PPO herbicide is selected from the group consisting of chlorphthalim, flufenpyr, flufenpyr-ethyl, flumipropyn, pyraclonil, profluazol, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate; cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate; methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} propanoate (flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} propanoate, methyl 2-{1[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{1[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy} propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy} propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, 2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, {[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid, 2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, ethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetic acid, ethyl (2-{2-chloro-5-[4-(1,1-difluoroethyl)-3-methyl-2,6-dioxo-3,6-dihydropyrimidin-1(2H)-yl]-4-fluorophenoxy}phenoxy)acetate, (2-{2-chloro-5-[4-(1,1-difluoroethyl)-3-methyl-2,6-dioxo-3,6-dihydropyrimidin-1(2H)-yl]-4-fluorophenoxy}phenoxy)acetic acid, ethyl (2-{2-chloro-4-fluoro-5-[4-(1-fluoroethyl)-3-methyl-2,6-dioxo-3,6-dihydropyrimidin-1(2H)-yl]phenoxy}phenoxy)acetate, 2-methoxyethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, tetrahydrofuran-2-ylmethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, cyanomethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate, methyl (2-{2-chloro-5-[4-(1,1-difluoroethyl)-3-methyl-2,6-dioxo-3,6-dihydropyrimidin-1(2H)-yl]-4-fluorophenoxy}phenoxy)(methoxy)acetate, methyl (2-{2-bromo-5-[4-(1,1-difluoroethyl)-3-methyl-2,6-dioxo-3,6-dihydropyrimidin-1(2H)-yl]-4-fluorophenoxy}phenoxy)(methoxy)acetate, [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetic acid, ethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, 2-methoxyethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, tetrahydrofuran-2-ylmethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate, ethyl 2-[[3-[5-[4-(1,1-difluoroethyl)-3-methyl-2,6-dioxo-pyrimidin-1-yl]-4-fluoro-2-nitro-phenoxy]-2-pyridyl]oxy]acetate, 1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-{1[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid, 1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, 2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, [({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid, 1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid, allyl 1-[2-chloro-5-(3,5-dimethyl-2,6-dioxo-4-sulfanylidene-1,3,5-triazinan-1-yl)-4-fluorophenoxy]cyclopropanecarboxylate, 1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate, 2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]cyclopropanecarboxylic acid, methyl 1-[2-chloro-5-(3,5-dimethyl-2,6-dioxo-4-sulfanylidene-1,3,5-triazinan-1-yl)-4-fluorophenoxy]cyclopropanecarboxylate, 1-[2-chloro-5-(3,5-dimethyl-2,6-dioxo-4-sulfanylidene-1,3,5-triazinan-1-yl)-4-fluorophenoxy]-N,N-dimethylcyclopropanecarboxamide, and ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate.

59. (canceled)

60. A method of producing a progeny cotton plant comprising cotton event Gh_CSM63718 comprising:
a) sexually crossing the cotton plant of claim 34 with itself or a second cotton plant;
b) collecting one or more seeds produced from the cross;
c) growing one or more seeds to produce one or more progeny plants; and
d) selecting at least a first progeny plant or seed comprising cotton event Gh_CSM63718.

61. An inbred or hybrid cotton plant or seed comprising cotton event Gh_CSM63718 produced by the method of claim 60.

62. A nonliving or nonregenerable cotton plant material or a commodity product comprising:
a) a recombinant DNA molecule comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing;
b) the DNA construct of claim 29, or
c) cotton event Gh_CSM63718, a representative sample of seed comprising cotton event Gh_CSM63718 having been deposited under ATCC Accession No. PTA-127638.

63-64. (canceled)

65. The commodity product of claim 62, wherein:
a) the commodity product is produced from a transgenic cotton plant, plant part, plant seed, or plant cell comprising cotton event Gh_CSM63718; or
b) the commodity product comprises whole or processed seeds; viable or nonviable seeds; viable plant parts (such as roots, nodes, bolls, buds or leaves); viable plant cells; processed plant parts; processed plant tissues; dehydrated plant tissues; dehydrated plant parts; frozen plant tissues; frozen plant parts; food for human consumption such as cottonseed oil; plant parts processed for animal feed such as cottonseed meal and cottonseed hulls; cotton fiber; or cotton linters.

66. (canceled)

67. A method of producing a commodity product, the method comprising:
a) obtaining the cotton plant, plant part, or plant seed of claim 34; and
b) producing a commodity product from the transgenic cotton plant, plant part, or plant seed.

68. A method of controlling, preventing, or reducing the development of herbicide-tolerant weeds comprising cultivating in a crop growing environment a cotton plant comprising transgenes that provide tolerance to glufosinate, ß-triketone HPPD inhibitor herbicides, dicamba, glyphosate and PPO herbicides, a cotton plant comprising the DNA construct of claim 29, or a cotton plant comprising event Gh_CSM63718.

69. The method of claim 68, wherein:
a) the method further comprises applying to the crop growing environment at least one herbicide selected from the group consisting of glufosinate, a ß-triketone HPPD inhibitor, dicamba, glyphosate, a PPO herbicide, and any combination thereof, wherein the cotton plant is tolerant to the at least one herbicide; or
b) the transgenes that provide tolerance to the herbicides are present at a single genomic location in the cotton plant.

70. (canceled)

71. A method of reducing loci for cotton breeding by inserting a construct comprising transgenes that provide tolerance to glufosinate, ß-triketone HPPD inhibitor herbicides, dicamba, glyphosate and PPO herbicides or the construct of claim 29 as a single locus at a genomic location in a cotton plant.

72. A cotton plant, plant cell, plant part, plant seed, nonliving or nonregenerable cotton plant material, or commodity product comprising the DNA construct of claim 29 or cotton event Gh_CSM63718, wherein the cotton plant, plant cell, plant part, plant seed, nonliving or nonregenerable cotton plant material, or commodity product;
a) further comprises one or more transgenes for providing resistance to insect infestations;
b) further comprises cotton event Gh_BCS246002 or cotton event MON15947;
c) further comprises a recombinant DNA molecule comprising a sequence selected from the group consisting of SEQ ID NO:212; SEQ ID NO:213; SEQ ID NO:214; SEQ ID NO:215; SEQ ID NO:216; SEQ ID NO:217; SEQ ID NO:218; SEQ ID NO:219; SEQ ID NO:220; SEQ ID NO:221; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:212 or the full length of SEQ ID NO: 213; and a complete complement of any of the foregoing;
d) further comprises one or more transgenes for providing resistance to insect infestations, wherein the transgenes for providing resistance to insect infestations are selected from the group consisting of Cry1B.3, Cry1 Da 7, Vip3Cb1.1, Cry2Ab2, and combinations of any thereof;
e) further comprises one or more transgenes for providing resistance to insect infestations, wherein the transgenes for providing resistance to insect infestations are selected from the group consisting of Cry1B.3, Cry1 Da 7, Vip3Cb1.1, Cry2Ab2, and combinations of any thereof, and wherein the Cry1B.3 transgene comprises a polynucleotide sequence encoding a protein having the amino acid sequence of SEQ ID NO:225; the Cry1 Da 7 transgene comprises a polynucleotide sequence encoding a protein having the amino acid sequence of SEQ ID NO:227; the Vip3Cb1.1 transgene comprises a polynucleotide sequence encoding a protein having the amino acid sequence of SEQ ID NO:229; and the Cry2Ab2 transgene comprises a polynucleotide sequence encoding a protein having the amino acid sequence of SEQ ID NO:231;
f) further comprises one or more transgenes for providing resistance to insect infestations, wherein the transgenes for providing resistance to insect infestations provide resistance to infestations by Lepidopteran pests selected from the group consisting of Cotton Bollworm (Helicoverpa zea), Tobacco Budworm (Heliothis virescens), Fall Armyworm (Spodoptera frugiperda), Old World Bollworm (Helicoverpa armigera), and combinations of any thereof; or
g) further comprises cotton event Gh_BCS246002, wherein the transgenes in event Gh_BCS246002 for providing resistance to insect infestations are present at a single genomic location in the cotton plant.

73-78. (canceled)

79. A cotton plant, plant cell, plant part, seed, nonliving or nonregenerable cotton plant material, or cotton commodity product, comprising a foreign DNA at an insertion site in the cotton genome, the insertion site having a nucleic acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the sequence of SEQ ID NO:13.

80. The cotton plant, plant cell, plant part, seed, nonliving or nonregenerable cotton plant material, or cotton commodity product of claim 79, wherein:
a) the foreign DNA is flanked by 5′ and 3′ flanking regions, wherein said 5′ flanking region is upstream of and contiguous with said foreign DNA and comprises SEQ ID NO:11, and wherein said 3′ flanking region is downstream of and contiguous with said foreign DNA and comprises SEQ ID NO:12;
b) the foreign DNA comprises at least one herbicide tolerance gene selected from the group consisting of a phosphinothricin N-acetyltransferase (PAT) gene, a triketone dioxygenase (TDO) gene, a dicamba monooxygenase (DMO) gene, a 5-enolpyruvylshikimate-3-phosphate synthase (EPSPS) gene, a protoporphyrinogen oxidase (PPO) gene, and any combination of any thereof;
c) the foreign DNA comprises a phosphinothricin N-acetyltransferase (PAT) gene, a triketone dioxygenase (TDO) gene, a dicamba monooxygenase (DMO (EPSPS) a 5-enolpyruvylshikimate-3-phosphate synthase (EPSPS) gene, and a protoporphyrinogen oxidase (PPO) gene;
d) the foreign DNA comprises SEQ ID NO: 9;
e) the foreign DNA comprises SEQ SEQ ID NO:9 with one or more modifications;
f) the foreign DNA comprises SEQ ID NO:9 with one or more modifications and wherein the one or more modifications comprise one or more insertions, one or more deletions, one or more substitutions, or a combination of any thereof, within SEQ ID NO:9;
g) the foreign DNA comprises SEQ ID NO:9 with one or more modifications comprising a deletion of all or a portion of the phosphinothricin N-acetyltransferase (PAT) gene, a deletion of all or a portion of the triketone dioxygenase (TDO) gene, a deletion of all or a portion of the dicamba monooxygenase (DMO) gene, a deletion of all or a portion of the 5-enolpyruvylshikimate-3-phosphate synthase (EPSPS) gene, a deletion of all or a portion of the protoporphyrinogen oxidase (PPO) gene, or a combination of any thereof; or
h) the foreign DNA comprises SEQ ID NO:9 with one or more modifications comprising an insertion of an additional expression cassette.

81-87. (canceled)

88. A method of producing the cotton plant, plant cell, plant part, or seed of claim 79, said method comprising inserting foreign DNA at an insertion site in the genome of the cotton plant, plant cell, plant part, or seed, the insertion site having a nucleic acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the sequence of SEQ ID NO:13.

89. The method of claim 88, wherein the cotton plant produced by the method has improved trait efficacy, increased expression of one or more transgenes, improved stability of one or more transgenes, improved agronomic characteristics, or any combination thereof, as compared to a plant that has the same foreign DNA inserted at a different insertion site in the genome.

90. A method of producing a cotton plant or seed, said method comprising obtaining the cotton plant or seed of claim 34, and inserting foreign DNA into the sequence of SEQ ID NO:10 of the plant or seed, or deleting all or a portion of the sequence of SEQ ID NO:10 in the plant or seed.

91. (canceled)

92. A cotton plant or seed produced by the method of claim 88.

93. A method of detecting the presence a nucleic acid molecule encoding PPO_H_N90 in a sample derived from a cotton seed, plant, plant part or plant cell, progeny plant, or commodity product, the method comprising:
a) contacting the sample with a pair of DNA molecules; performing an amplification reaction sufficient to produce a DNA amplicon comprising the nucleic acid encoding the PPO_H_N90 or a portion of the nucleic acid encoding the PPO_H_N90 of sufficient length to identify the presence of the nucleic acid encoding the PPO_H_N90; and detecting the presence of the DNA amplicon, wherein the presence of the DNA amplicon indicates the presence of PPO_H_N90 in the sample; or
b) contacting the sample with a DNA molecule that functions as a DNA probe specific for the nucleic acid molecule encoding PPO_H_N90; subjecting the sample and the DNA molecule that functions as a probe to stringent hybridization conditions; and detecting the hybridization of the DNA molecule that functions as a probe to a DNA molecule in the sample, wherein the hybridization of the DNA molecule that functions as a probe to the DNA molecule in the sample is diagnostic for the presence of PPO_H_N90 in the sample.

94. The method of claim 93, wherein:
a) the pair of DNA molecules comprises SEQ ID NO:234 and SEQ ID NO:235; or
b) the DNA molecule that functions as a probe comprises SEQ ID NO:236.

95-96. (canceled)


SEQUENCE LISTING (237 sequences)

SEQ ID NO: 1 - DNA, 30 bp, Synthetic construct
  source 1..30: mol_type = other DNA
tcttaaaaatataattgatggtcaatatgg

SEQ ID NO: 2 - DNA, 30 bp, Synthetic construct
  source 1..30: mol_type = other DNA
tgatgatgctgactgccatacctaatgcct

SEQ ID NO: 3 - DNA, 60 bp, Synthetic construct
  source 1..60: mol_type = other DNA
aatttgttaccgaaatcttaaaaatataattgatggtcaatatggagaaaaagaaagagt

SEQ ID NO: 4 - DNA, 60 bp, Synthetic construct
  source 1..60: mol_type = other DNA
gcctaacttttggtgtgatgatgctgactgccatacctaatgcctcttcgatttgagatg

SEQ ID NO: 5 - DNA, 100 bp, Synthetic construct
  source 1..100: mol_type = other DNA
cacttatgattctattttgaaatttgttaccgaaatcttaaaaatataattgatggtcaatatggagaaaaagaaagagtaattaccaattttttttcaa

SEQ ID NO: 6 - DNA, 100 bp, Synthetic construct
  source 1..100: mol_type = other DNA
tctaatttcaaactattcgggcctaacttttggtgtgatgatgctgactgccatacctaatgcctcttcgatttgagatgtttaacaatgaaagtggtac

SEQ ID NO: 7 - DNA, 1050 bp, Synthetic construct
  source 1..1050: mol_type = other DNA
acctacttttttttttttcattcaaaagcttatggttcagtagttattgtgtctctcttatgatggggtgtaccaagcttgcaacccacactctttccctttcctcccccagcttgaggactttcgatttttgcaagtgccattttttttaaataaaaaagaaaaagtagatgggaatatatttttttttcacttttataataaagaattttatattatttattaatataggtgggggtagttattctcaaaatacacatctaccacgtattgttgtaacaattttacagaaaaaacaagtatataattattgctatttcaagttatttgattttaagatggtatattttgaaacattgctccatctttattaccattcaaactaatctaattcatatttccattccaaccaacttctccgtttggaaagaatataagatattgcaaaggatcataactaaaataatatttacatttatgttgtcttcttgaattacactatctctttccaatcaggcttggttttagggttgtttgagagtgcaattgtctagggtccacgggtggaaggggctcaaaaaaatttttttttcagcttttaatgtgggaaaaataatttctaaaacaaattaattaaatgtctaaagctttcaatttttcaccataaatgcttcatctcttagggctccaaaattttttcttttattgtattgcattatatatatatattaaaagaagtccaatttattatttcgcctagaacctcaaaaacatcaacaatggacctgaatgttcgactttatcccttaaaccatataacaacaattataaatctataaataaggagttgttacaatgtgaatttcaaaatcctaaaaaaaatataacttttattctcgttaaagactcattatattctagaaatttaattagaatcataacaattgtttcacttatgattctattttgaaatttgttaccgaaatcttaaaaatataattgatggtcaatatggagaaaaagaaagagtaattaccaattttttttcaa

SEQ ID NO: 8 - DNA, 1050 bp, Synthetic construct
  source 1..1050: mol_type = other DNA
tctaatttcaaactattcgggcctaacttttggtgtgatgatgctgactgccatacctaatgcctcttcgatttgagatgtttaacaatgaaagtggtacccgatttcaggttaattgacaggaaaaaaaaagcacctcattttaaaacttttttttaccacaatagttacattaatcaagtttttttccactcatgtaacggagaaagcgaaataacacaatatttctcctagtaaaagagatccctattagtgaaaaaagttttcaaagctaatacataaaatttgaatgttttattttttaataaaatgagaaaataatttattttagaagtaaccaaattaatttaatattttctacattaagggataagtagttatctacttacttattattttcaacaatttaaaaaaaaattatattaaataaatagttaatatgattatcaaacaaaattaaatgttgaaaactttcattaaaattttaataatttatgaaacacctaaagctaatatcctgcaatgttttctttttgaagaatttgcactattcaacatcaaatttgctatgtcacacctaaattcataataagtatcaatttcacatgaatgtataaattttttttcacctcaaatgaatttagcagctatatcctcgttgttcaacggataatcctcaatgttgaaacaaccagaaaagaggttgaacatgttggacctaccataaaaaattaaaaatttaattattattataaaatcataagaaatttcaaattattttaaaattataaaaattatattttaaaaaatactaatgacccaaaacatattaaataaataaaaattctctttttttaaaaaataaaaattaaattaatctaatttttcggttttaaatgattcttttgatttttagttttcgagtttttactggttcaaaacggttttaattattttcaattttatgtcactagttggactattattttttttcagtcgattcatctaatcaaattttgatatcatattaatatcatattgaataaactc

SEQ ID NO: 9 - DNA, 16736 bp, Synthetic construct
  source 1..16736: mol_type = other DNA
tgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaagtactatataacttcgtataatgtatgctatacgaagttatttcgctaccggtagcgacttaggctgagcccgggcaaggccttttcacctttggacaatcattgacagtaacacaacgaacattaactgcatcgtaaaataaaatgtcactacaatttgataaaatattgtgtaaaagagagtacttaaaaaaaaatggcgtagaaagtaccatactcctattagctttcaacaagaaatagggaaagaaagatgactaaaacataaataaagaaagataggaaattgagccatattgttttcctttcttagatgtttcatgtggatattattttacttgatccttgtggaatttatgttatgctattattaactcttgtgtataaatagaatacaataccaatacttaaaatagacataaaaaacgatgtgagacaatagataattaaaccattcttacataatttattccttacaaaatataattcacagaatttacttttacataaaacagaacgcacacacaacacacttcacaatattctcaacctaaaaacagagtaatagagcttgaatatgtgtttaattaatcaaatctgggtaactggtctaacgggtcttggaggtgctggaagttcgaaatcccgttgccagaatccaacgtcgtgccatcctccgtgtttgtacccggcggccctcaatgttccgcgtgccgtgtagcctagtgcttcatgtaggcgcacagaaggatcgttaggtagtccgatgacggccaccactgacttaaagccctgagcctccatagatttgagtaagtgggtgtacagggtgctgcccagacccagcctctgatggcgatgcgagacgtaaacagtggattccacagtccagtcgtaagcatttcgagccttccacggtccagcgtaggcgattcccgcgacgacgccttcgacctctgccacgagccacggatagcggtcttgtagtctctccaagtcatcaatccattcctgaggagtttgtggttcggtacgaaagtttactgtggatgtctcgatgtagtgatttacgatgtcacatactgccgccatgtcggcggctgttgcgggtctaatctctactggccgtcgctcgggagacatggtaccatctcaagactgcgacgagtatgcgtcacaacacaagaagctgaagagaggaattagattggtagaagaagaagcttagagagatgttggtgtgagcaagaaactgagagatttataaagcgagtgatcaacaaccaattcgttatctcttcgtcttcaaggcttaactctctgatcgccacacgatgcggattggccaagttgccacgaggaagatgattaggaggattctattttgattggtccacgtggaatatgtttatgaggttgtcgtcaaatggctaatctgtgccaatggatttgttttttttttcaagtgaagattataacattatttattaccacatatgctttggaaaatcattgtttttttttttttttgaggattgaggtaagccattcatatgtgatcaagatacttctacactcttcttcatacgataacattccaaaattttggataatgacattgcttatcgataataatgttccaaataaccgatgtaaactgattggtggagagaaattggcttagccaatagaatataattctaattttgttatatacaactctagatacaacaggtatcaagtatgtggttatcaaattaaccatattgtactaaaccgctaaaaactaattaatgcaactttttctttacacaaaatgtaaaaacaagcgatgcaatgcatacgtaagatttgataaatatatagtttagaagatgaagtaaagatagtcgaggctaagatagcatcatgcaaaatatcatatgctaaaatatgaaataagattatagtgtttaaattcatagtgaaaatatgaatatatcagaaattcatatgaatttacttgttttaaaataaaaatgataactatatcatagtttattgttccctcaaagttactataaaaacaactcatctaaaactatatattcacaatcacaagcaatcgttttctacactaagctataaaatgaaacacttcacttgtctgatcattataatttctcatcctcttcattattttcagttttctcaaccattctctcatgtggaacgagccatcatatcatcaatggacaaaaccaaattttaattttaggcttcattcgctgccgacgaagcaatatgtacatgttctacatgcaagacaagagaacaaagattactgatatgatattaaactattattgatccatatataagacatgttgaagaaccaaacgattgataataatgtggtggagctactcgcacaatgctaaatgttaactgtatgtgacaaaattagcaaaagtttgcgtgccaggtttgattcttagtacacatacatgacttttaacatactgaggttagaaaatgaagtacagtaaaacctttataaattaataatgttgggactgcaaaattttattaatttaaaaaggttttaatttatcgataaattaatattttttaattatagagaaattttcctaatttggtaactttttaaaaagttttataagatttttttcattatagaaaatatctttctaaaatcaattacaagtacaaaattatatcatgtctagaaaacaataccaaattttttttgatacagtaacacattagaactggcgcgccgaagcccgctttacaagtggccagctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattggatcctttgtatctattttatctattaaaggtatgtttgggcttcaggttggaatggagtagaattgaatgggttggggagtaaattttccattcaacaagttcaatttcaaaatggctaataagttttgaactcaattttattttcaataaattccttaattttttgttccttgtttgtaaactattgacttattcgatatattttaaaattgaggtattttaaaaaaataatacaatattaaaattatttataaaatataacaaaatttatgtatagtttatttgaaaattttactatagtttcatttttatattattcctaaccatttccatttaaaattatttcaattatttcttttattaatataattgaaatttcatggatttattagacacatgatttgaaattttatgggtttattaagtattttctaacacaaaatcgcttccgcatcgttttcaattcattcagtaatagaagtaattttttaaaagaaccaaatttgccaaattttgagttccataaggactctgaaaactcattatgtctattactcttcactaattgtagagacttaaattcaagaaaagagacactaattgatgataattgcccaaaaaataaaaataaaaatgtttcttccccatcctcaacctccatgaattcacagagcccaaagattaattattgggccccaattcctactcatatataccttacagtccctcaaagaaatcttaggaagtaatcaatttctgtttattcaagatgtagcctcccaaaagaaaaatacatcacatcaaattcaaacaaaaatatctacagctagcaaaacctcaaaccgttaaaatttcaagccacataaatgaaattttcatctgaaaaaaggacaatctatctagacgttagatttcagccctaatatgaatctgaagcatttggtggacgagaaagagccatgtaggaatgcatcaaacaaaggaaaaatctttgaactccaatgggattgaagatacagataccaatggataagaatctgttctctttgcccactatttaaactcaccaaacccaccagtatcttcctcaccacaaaatacattccaccgttgatcacaagccttattccaccacctccaacaggcgcgccatggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctgaggtacctaagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctcgcgatcgcgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtatgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtgagagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagttacctgcaggaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaagaattcgatatcaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagagatctaccatggcggcactggtgacctcccagctcgcgacaagcggcaccgtcctgtcggtgacggaccgcttccggcgtcccggcttccagggactgaggccacggaacccagccgatgccgctctcgggatgaggacggtgggcgcgtccgcggctcccaagcagagcaggaagccacaccgtttcgaccgccggtgcttgagcatggtcgtctgcatgcttcacggtgcttcttccagacccgcaacggctagaaagagttctggcttgtctggaaccgtccgtattccaggagacaaaagcattagtcaccgctctttcatgtttggtgggctggcatctggagagacgcgcatcactggtcttctggaaggagaggacgtcatcaatacagggaaggcaatgcaggctatgggtgcccgtattcgcaaggaaggtgatacttggatcatagacggagttgggaacggtggcttacttgcaccggaggctcctctcgactttggcaacgcagccacagggtgtagacttactatgggcctcgtgggtgtttacgatttcgattcaacctttattggggatgcctctctcactaaacgcccaatgggaagagtccttaacccgttgagggagatgggcgtacaagttaagtccgaggacggcgacagattgcccgtcaccttgcgcggccctaagacacccacccctattacttacagggttccaatggcatctgctcaagtgaagtccgcagttctgctcgctggattgaacacaccgggtattactaccgtgattgagccgatcatgactcgtgaccacactgagaagatgcttcagggtttcggtgctaacctcaccgttgaaacagacgcggacggtgtgaggaccattcgcctggagggaaggggaaaactcactggtcaagtcattgacgtgcccggtgatccctccagcacggcgttcccactggttgccgctcttctcgtaccaggctccgatgtgacaattctaaacgtcctcatgaatcctactagaaccggattgatacttacattgcaggaaatgggtgctgatattgaagttatcaatcctagactagccggaggtgaggacgtagctgatttgcgggtgaggtcttctacattgaaaggtgttaccgtacctgaagatagggcaccttcaatgattgacgagtatccaattcttgccgtcgcggctgcctttgctgagggcgcgaccgtgatgaatggactagaggagttgagagtgaaggaatccgacagattgagcgcagtcgctaacggacttaaactcaatggcgttgattgtgatgagggtgagactagcttggtagtccgtgggcgaccagacggaaagggtttgggcaacgcttcgggtgctgccgttgcaactcacttggatcatcggatagcgatgagttttctggtgatgggtctcgtaagcgagaatcctgtgacagtcgacgatgcaactatgatcgctacttccttccctgagtttatggatttaatggcaggactaggtgcaaagattgaactctctgataccaaagcggcctaaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatgcggccgcttaactaactagtgcacttaatctgcggagtcggggtacggacgttcaagacgccccgtgagtggcggtacggttggcccccgcaggtcggacttccagcttatcgctagtctcaataacccctcggcgctctcgcaggcgctcagtgaaataagtgcgtgctaactcaacgctggggcgaacagagttacagggatattatttcgtacagtacgggagccctaccttatacacgggctgggcagtgaccgcctgccgagtcccacgatttgaggacgctggggaaaactccactgcagagcggtgatacaggggggaccgttctggcccttgttaccccgatcgctaaattaacgtgagaacccttggcagaccgtcacaacgtgctaaaaggtcggagtagtgcctgggggctcggatcaggcttagtcactgcccgggcttcccaccgcaacccttagtgacacacccgcttcagtatagcacaatcctctctgcacaccgcgatcggttggggcagtaggcgcttacaaataaacgattgcctctctcgcgtctgatccgtgctcacttgtctctagccaactccgcgaagagcacccggagtcggaaaagggggcggttagtgtggagcgatattgctgatccggtcagaggttaggaatccaggataccgaggcgggttggccgtcctctcccctatcctggactttcgctttgcgcctacagcctcagggccgacccggagcacgtgccacgcgagtaattagtccgaggtgacgcccagtttcaaactctttcacttgagggttccttctgttcagcccacgcgggtaggccccaagcccgccaactaggcccgcaccttcttgtcgggttcaccaatcgctaggtcggcgtaattacgcgtgcgctcagcccggagtgccagggagggccccaccgaggcccggtagcggcaacaaacgggaataatttaggtctggcgagtcccgaaaacctgggaggaagcttagttagctaggtttaaacagagtatactgatcaagcaatgttaaattaacaatacatgcaatatcattaaagtaaaatacataacataagcttcgttaaaacataaatcaagccaagtgacattaaaatttaaaaacaacattcaaaattaatttcactacaacataaactataatcattcagaatctctatgtatgcaaagagattcaccattggtgcatggaggaatgcacgcacattattcactcaccatcttttttttattaaccatataattaagatataccatgaaattactgcatgatgatggcacatgcaccagtgtcatatggtctagagggccttcttgtacgatagctttgcaaaatcctccgcgaacttcttaacctgctcccaatccgtgtactcgacctccttgctcgtgtctgtctctcccccagtcatccgcattattagctggatcatcaccttgtcgatccaccggtatcgcgggtaccgaagggcccctgcgaacactccgcacaacgcgggctgccagggggtagcaagcaagaattttcggacataagggtttgtctggggagtacgcttctcgggcttccttgctgtgaggtttacgcagaagaacgcgcttggcatgttgttcagctgatccacgtttctcttgatgaacttgtcaagcacggcgttgaagtggccgtaacgaatactcgcaccgattagcacctgatcgtattgggtgaggttcacgtgctccccgtgggtgaggtcgatcacgtcgcattcggccttctccttcatgcaggaggcgatgtatgaagcaattgcgtgggtctggccgtcccgcgtcgagtacagtaccaaggccttcatgcacctgacaacgaatggcctatggtccaggcgccgggtaaggcgtgcggactgtttcagcttcaggctcttgaacgaagacggcttagcggggaacgcagcagaaggctggaggtggctgaaggagtagatcctgcgcgtctcagtgccgacgctcaccacgccggagaacgcagcggtggcggtagtggtggcggtggccatggtacctgtgaaatattaaaaagaacaaaactttgatgaataatcaaaagttatcatgttccaagaaaaaaaaaatcgatcctttcaacagagaaatgttgtagatttgatccaataagacgcttcagatattatggagaaaaatcaatttcaaatttgaccaatcgatctaccaaatcaattcaaaattaggtatcggttcaaaaattggaatccgggtgaaattatactgaatctcaaaaccaatctacgaatctataaactcatcgaggcgttctccaaaatcaaatcagaaaacctagatcgtcgtcaatggaaaatggtaaaaatagagaggagagaaattacctgtttccgggatcttccttgttctttttctctgcaagtgttgagactctttccaagagggttatgtgactcactgcggtccgtggtttagctgagagttgaggagtgacgagagaaatatagggtttatatatatagggaactaggtcaattagggtttaatattctcgttgggccggcctttattttagatctaaagcccaaactcttaatgcccagataaaatgttctcatatttttagtgttgtgtttttaattgccttacaccattgtttcatttataaattttaatccaaatttattaatttggcagattaatttgaattatatatattatatgtatttctctctgattgaaaataatcgcatagaaaaggggaaatttttttatcatttgttctaacataaaagttgagttaattctaagattattttgtttcaacaagaaaagtaaatcttattacataaatctatttttactacaatcgaaaaccaaaacaaaaactgtccaaaaaattctataacacacaaaataaccttattttgtcggttgtaattatgactagttcggataacattaacaatagacagaggtgtggataaggtgatgaggtgtgggattacatgtggttggttggattttccttatccatctgtggttggcattttgtggctttgatgtggttcttttgcttatcttttgataaggtgatgaaaatactggttgccacgtggcaatatcttacttttgataattgattggatagtgacttattaaacaatttgtgacaagtgtttatcgtaattgcattttcttattccttaattgtatgctggttattttcctagttcagaattttcttaggtgtgaaatttatattcttggtaaaaaggagtctcaaaacatgatattaattaatactttttgtgctttttcattttgtgaagatgatatttaaatttcaataaccaaaaatatatatattgctcttgtatgattgtactcgtacgcagggataacagggtaataggtctcacgcggcaaatcctaccacctcaaccgttatagttacgaaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgggcctaacttttggtgtgatgatgctgactg

SEQ ID NO: 10 - DNA, 18736 bp, Synthetic construct
  source 1..18736: mol_type = other DNA
acctacttttttttttttcattcaaaagcttatggttcagtagttattgtgtctctcttatgatggggtgtaccaagcttgcaacccacactctttccctttcctcccccagcttgaggactttcgatttttgcaagtgccattttttttaaataaaaaagaaaaagtagatgggaatatatttttttttcacttttataataaagaattttatattatttattaatataggtgggggtagttattctcaaaatacacatctaccacgtattgttgtaacaattttacagaaaaaacaagtatataattattgctatttcaagttatttgattttaagatggtatattttgaaacattgctccatctttattaccattcaaactaatctaattcatatttccattccaaccaacttctccgtttggaaagaatataagatattgcaaaggatcataactaaaataatatttacatttatgttgtcttcttgaattacactatctctttccaatcaggcttggttttagggttgtttgagagtgcaattgtctagggtccacgggtggaaggggctcaaaaaaatttttttttcagcttttaatgtgggaaaaataatttctaaaacaaattaattaaatgtctaaagctttcaatttttcaccataaatgcttcatctcttagggctccaaaattttttcttttattgtattgcattatatatatatattaaaagaagtccaatttattatttcgcctagaacctcaaaaacatcaacaatggacctgaatgttcgactttatcccttaaaccatataacaacaattataaatctataaataaggagttgttacaatgtgaatttcaaaatcctaaaaaaaatataacttttattctcgttaaagactcattatattctagaaatttaattagaatcataacaattgtttcacttatgattctattttgaaatttgttaccgaaatcttaaaaatataattgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaagtactatataacttcgtataatgtatgctatacgaagttatttcgctaccggtagcgacttaggctgagcccgggcaaggccttttcacctttggacaatcattgacagtaacacaacgaacattaactgcatcgtaaaataaaatgtcactacaatttgataaaatattgtgtaaaagagagtacttaaaaaaaaatggcgtagaaagtaccatactcctattagctttcaacaagaaatagggaaagaaagatgactaaaacataaataaagaaagataggaaattgagccatattgttttcctttcttagatgtttcatgtggatattattttacttgatccttgtggaatttatgttatgctattattaactcttgtgtataaatagaatacaataccaatacttaaaatagacataaaaaacgatgtgagacaatagataattaaaccattcttacataatttattccttacaaaatataattcacagaatttacttttacataaaacagaacgcacacacaacacacttcacaatattctcaacctaaaaacagagtaatagagcttgaatatgtgtttaattaatcaaatctgggtaactggtctaacgggtcttggaggtgctggaagttcgaaatcccgttgccagaatccaacgtcgtgccatcctccgtgtttgtacccggcggccctcaatgttccgcgtgccgtgtagcctagtgcttcatgtaggcgcacagaaggatcgttaggtagtccgatgacggccaccactgacttaaagccctgagcctccatagatttgagtaagtgggtgtacagggtgctgcccagacccagcctctgatggcgatgcgagacgtaaacagtggattccacagtccagtcgtaagcatttcgagccttccacggtccagcgtaggcgattcccgcgacgacgccttcgacctctgccacgagccacggatagcggtcttgtagtctctccaagtcatcaatccattcctgaggagtttgtggttcggtacgaaagtttactgtggatgtctcgatgtagtgatttacgatgtcacatactgccgccatgtcggcggctgttgcgggtctaatctctactggccgtcgctcgggagacatggtaccatctcaagactgcgacgagtatgcgtcacaacacaagaagctgaagagaggaattagattggtagaagaagaagcttagagagatgttggtgtgagcaagaaactgagagatttataaagcgagtgatcaacaaccaattcgttatctcttcgtcttcaaggcttaactctctgatcgccacacgatgcggattggccaagttgccacgaggaagatgattaggaggattctattttgattggtccacgtggaatatgtttatgaggttgtcgtcaaatggctaatctgtgccaatggatttgttttttttttcaagtgaagattataacattatttattaccacatatgctttggaaaatcattgtttttttttttttttgaggattgaggtaagccattcatatgtgatcaagatacttctacactcttcttcatacgataacattccaaaattttggataatgacattgcttatcgataataatgttccaaataaccgatgtaaactgattggtggagagaaattggcttagccaatagaatataattctaattttgttatatacaactctagatacaacaggtatcaagtatgtggttatcaaattaaccatattgtactaaaccgctaaaaactaattaatgcaactttttctttacacaaaatgtaaaaacaagcgatgcaatgcatacgtaagatttgataaatatatagtttagaagatgaagtaaagatagtcgaggctaagatagcatcatgcaaaatatcatatgctaaaatatgaaataagattatagtgtttaaattcatagtgaaaatatgaatatatcagaaattcatatgaatttacttgttttaaaataaaaatgataactatatcatagtttattgttccctcaaagttactataaaaacaactcatctaaaactatatattcacaatcacaagcaatcgttttctacactaagctataaaatgaaacacttcacttgtctgatcattataatttctcatcctcttcattattttcagttttctcaaccattctctcatgtggaacgagccatcatatcatcaatggacaaaaccaaattttaattttaggcttcattcgctgccgacgaagcaatatgtacatgttctacatgcaagacaagagaacaaagattactgatatgatattaaactattattgatccatatataagacatgttgaagaaccaaacgattgataataatgtggtggagctactcgcacaatgctaaatgttaactgtatgtgacaaaattagcaaaagtttgcgtgccaggtttgattcttagtacacatacatgacttttaacatactgaggttagaaaatgaagtacagtaaaacctttataaattaataatgttgggactgcaaaattttattaatttaaaaaggttttaatttatcgataaattaatattttttaattatagagaaattttcctaatttggtaactttttaaaaagttttataagatttttttcattatagaaaatatctttctaaaatcaattacaagtacaaaattatatcatgtctagaaaacaataccaaattttttttgatacagtaacacattagaactggcgcgccgaagcccgctttacaagtggccagctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattggatcctttgtatctattttatctattaaaggtatgtttgggcttcaggttggaatggagtagaattgaatgggttggggagtaaattttccattcaacaagttcaatttcaaaatggctaataagttttgaactcaattttattttcaataaattccttaattttttgttccttgtttgtaaactattgacttattcgatatattttaaaattgaggtattttaaaaaaataatacaatattaaaattatttataaaatataacaaaatttatgtatagtttatttgaaaattttactatagtttcatttttatattattcctaaccatttccatttaaaattatttcaattatttcttttattaatataattgaaatttcatggatttattagacacatgatttgaaattttatgggtttattaagtattttctaacacaaaatcgcttccgcatcgttttcaattcattcagtaatagaagtaattttttaaaagaaccaaatttgccaaattttgagttccataaggactctgaaaactcattatgtctattactcttcactaattgtagagacttaaattcaagaaaagagacactaattgatgataattgcccaaaaaataaaaataaaaatgtttcttccccatcctcaacctccatgaattcacagagcccaaagattaattattgggccccaattcctactcatatataccttacagtccctcaaagaaatcttaggaagtaatcaatttctgtttattcaagatgtagcctcccaaaagaaaaatacatcacatcaaattcaaacaaaaatatctacagctagcaaaacctcaaaccgttaaaatttcaagccacataaatgaaattttcatctgaaaaaaggacaatctatctagacgttagatttcagccctaatatgaatctgaagcatttggtggacgagaaagagccatgtaggaatgcatcaaacaaaggaaaaatctttgaactccaatgggattgaagatacagataccaatggataagaatctgttctctttgcccactatttaaactcaccaaacccaccagtatcttcctcaccacaaaatacattccaccgttgatcacaagccttattccaccacctccaacaggcgcgccatggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctgaggtacctaagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctcgcgatcgcgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtatgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtgagagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagttacctgcaggaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaagaattcgatatcaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagagatctaccatggcggcactggtgacctcccagctcgcgacaagcggcaccgtcctgtcggtgacggaccgcttccggcgtcccggcttccagggactgaggccacggaacccagccgatgccgctctcgggatgaggacggtgggcgcgtccgcggctcccaagcagagcaggaagccacaccgtttcgaccgccggtgcttgagcatggtcgtctgcatgcttcacggtgcttcttccagacccgcaacggctagaaagagttctggcttgtctggaaccgtccgtattccaggagacaaaagcattagtcaccgctctttcatgtttggtgggctggcatctggagagacgcgcatcactggtcttctggaaggagaggacgtcatcaatacagggaaggcaatgcaggctatgggtgcccgtattcgcaaggaaggtgatacttggatcatagacggagttgggaacggtggcttacttgcaccggaggctcctctcgactttggcaacgcagccacagggtgtagacttactatgggcctcgtgggtgtttacgatttcgattcaacctttattggggatgcctctctcactaaacgcccaatgggaagagtccttaacccgttgagggagatgggcgtacaagttaagtccgaggacggcgacagattgcccgtcaccttgcgcggccctaagacacccacccctattacttacagggttccaatggcatctgctcaagtgaagtccgcagttctgctcgctggattgaacacaccgggtattactaccgtgattgagccgatcatgactcgtgaccacactgagaagatgcttcagggtttcggtgctaacctcaccgttgaaacagacgcggacggtgtgaggaccattcgcctggagggaaggggaaaactcactggtcaagtcattgacgtgcccggtgatccctccagcacggcgttcccactggttgccgctcttctcgtaccaggctccgatgtgacaattctaaacgtcctcatgaatcctactagaaccggattgatacttacattgcaggaaatgggtgctgatattgaagttatcaatcctagactagccggaggtgaggacgtagctgatttgcgggtgaggtcttctacattgaaaggtgttaccgtacctgaagatagggcaccttcaatgattgacgagtatccaattcttgccgtcgcggctgcctttgctgagggcgcgaccgtgatgaatggactagaggagttgagagtgaaggaatccgacagattgagcgcagtcgctaacggacttaaactcaatggcgttgattgtgatgagggtgagactagcttggtagtccgtgggcgaccagacggaaagggtttgggcaacgcttcgggtgctgccgttgcaactcacttggatcatcggatagcgatgagttttctggtgatgggtctcgtaagcgagaatcctgtgacagtcgacgatgcaactatgatcgctacttccttccctgagtttatggatttaatggcaggactaggtgcaaagattgaactctctgataccaaagcggcctaaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatgcggccgcttaactaactagtgcacttaatctgcggagtcggggtacggacgttcaagacgccccgtgagtggcggtacggttggcccccgcaggtcggacttccagcttatcgctagtctcaataacccctcggcgctctcgcaggcgctcagtgaaataagtgcgtgctaactcaacgctggggcgaacagagttacagggatattatttcgtacagtacgggagccctaccttatacacgggctgggcagtgaccgcctgccgagtcccacgatttgaggacgctggggaaaactccactgcagagcggtgatacaggggggaccgttctggcccttgttaccccgatcgctaaattaacgtgagaacccttggcagaccgtcacaacgtgctaaaaggtcggagtagtgcctgggggctcggatcaggcttagtcactgcccgggcttcccaccgcaacccttagtgacacacccgcttcagtatagcacaatcctctctgcacaccgcgatcggttggggcagtaggcgcttacaaataaacgattgcctctctcgcgtctgatccgtgctcacttgtctctagccaactccgcgaagagcacccggagtcggaaaagggggcggttagtgtggagcgatattgctgatccggtcagaggttaggaatccaggataccgaggcgggttggccgtcctctcccctatcctggactttcgctttgcgcctacagcctcagggccgacccggagcacgtgccacgcgagtaattagtccgaggtgacgcccagtttcaaactctttcacttgagggttccttctgttcagcccacgcgggtaggccccaagcccgccaactaggcccgcaccttcttgtcgggttcaccaatcgctaggtcggcgtaattacgcgtgcgctcagcccggagtgccagggagggccccaccgaggcccggtagcggcaacaaacgggaataatttaggtctggcgagtcccgaaaacctgggaggaagcttagttagctaggtttaaacagagtatactgatcaagcaatgttaaattaacaatacatgcaatatcattaaagtaaaatacataacataagcttcgttaaaacataaatcaagccaagtgacattaaaatttaaaaacaacattcaaaattaatttcactacaacataaactataatcattcagaatctctatgtatgcaaagagattcaccattggtgcatggaggaatgcacgcacattattcactcaccatcttttttttattaaccatataattaagatataccatgaaattactgcatgatgatggcacatgcaccagtgtcatatggtctagagggccttcttgtacgatagctttgcaaaatcctccgcgaacttcttaacctgctcccaatccgtgtactcgacctccttgctcgtgtctgtctctcccccagtcatccgcattattagctggatcatcaccttgtcgatccaccggtatcgcgggtaccgaagggcccctgcgaacactccgcacaacgcgggctgccagggggtagcaagcaagaattttcggacataagggtttgtctggggagtacgcttctcgggcttccttgctgtgaggtttacgcagaagaacgcgcttggcatgttgttcagctgatccacgtttctcttgatgaacttgtcaagcacggcgttgaagtggccgtaacgaatactcgcaccgattagcacctgatcgtattgggtgaggttcacgtgctccccgtgggtgaggtcgatcacgtcgcattcggccttctccttcatgcaggaggcgatgtatgaagcaattgcgtgggtctggccgtcccgcgtcgagtacagtaccaaggccttcatgcacctgacaacgaatggcctatggtccaggcgccgggtaaggcgtgcggactgtttcagcttcaggctcttgaacgaagacggcttagcggggaacgcagcagaaggctggaggtggctgaaggagtagatcctgcgcgtctcagtgccgacgctcaccacgccggagaacgcagcggtggcggtagtggtggcggtggccatggtacctgtgaaatattaaaaagaacaaaactttgatgaataatcaaaagttatcatgttccaagaaaaaaaaaatcgatcctttcaacagagaaatgttgtagatttgatccaataagacgcttcagatattatggagaaaaatcaatttcaaatttgaccaatcgatctaccaaatcaattcaaaattaggtatcggttcaaaaattggaatccgggtgaaattatactgaatctcaaaaccaatctacgaatctataaactcatcgaggcgttctccaaaatcaaatcagaaaacctagatcgtcgtcaatggaaaatggtaaaaatagagaggagagaaattacctgtttccgggatcttccttgttctttttctctgcaagtgttgagactctttccaagagggttatgtgactcactgcggtccgtggtttagctgagagttgaggagtgacgagagaaatatagggtttatatatatagggaactaggtcaattagggtttaatattctcgttgggccggcctttattttagatctaaagcccaaactcttaatgcccagataaaatgttctcatatttttagtgttgtgtttttaattgccttacaccattgtttcatttataaattttaatccaaatttattaatttggcagattaatttgaattatatatattatatgtatttctctctgattgaaaataatcgcatagaaaaggggaaatttttttatcatttgttctaacataaaagttgagttaattctaagattattttgtttcaacaagaaaagtaaatcttattacataaatctatttttactacaatcgaaaaccaaaacaaaaactgtccaaaaaattctataacacacaaaataaccttattttgtcggttgtaattatgactagttcggataacattaacaatagacagaggtgtggataaggtgatgaggtgtgggattacatgtggttggttggattttccttatccatctgtggttggcattttgtggctttgatgtggttcttttgcttatcttttgataaggtgatgaaaatactggttgccacgtggcaatatcttacttttgataattgattggatagtgacttattaaacaatttgtgacaagtgtttatcgtaattgcattttcttattccttaattgtatgctggttattttcctagttcagaattttcttaggtgtgaaatttatattcttggtaaaaaggagtctcaaaacatgatattaattaatactttttgtgctttttcattttgtgaagatgatatttaaatttcaataaccaaaaatatatatattgctcttgtatgattgtactcgtacgcagggataacagggtaataggtctcacgcggcaaatcctaccacctcaaccgttatagttacgaaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgggcctaacttttggtgtgatgatgctgactgccatacctaatgcctcttcgatttgagatgtttaacaatgaaagtggtacccgatttcaggttaattgacaggaaaaaaaaagcacctcattttaaaacttttttttaccacaatagttacattaatcaagtttttttccactcatgtaacggagaaagcgaaataacacaatatttctcctagtaaaagagatccctattagtgaaaaaagttttcaaagctaatacataaaatttgaatgttttattttttaataaaatgagaaaataatttattttagaagtaaccaaattaatttaatattttctacattaagggataagtagttatctacttacttattattttcaacaatttaaaaaaaaattatattaaataaatagttaatatgattatcaaacaaaattaaatgttgaaaactttcattaaaattttaataatttatgaaacacctaaagctaatatcctgcaatgttttctttttgaagaatttgcactattcaacatcaaatttgctatgtcacacctaaattcataataagtatcaatttcacatgaatgtataaattttttttcacctcaaatgaatttagcagctatatcctcgttgttcaacggataatcctcaatgttgaaacaaccagaaaagaggttgaacatgttggacctaccataaaaaattaaaaatttaattattattataaaatcataagaaatttcaaattattttaaaattataaaaattatattttaaaaaatactaatgacccaaaacatattaaataaataaaaattctctttttttaaaaaataaaaattaaattaatctaatttttcggttttaaatgattcttttgatttttagttttcgagtttttactggttcaaaacggttttaattattttcaattttatgtcactagttggactattattttttttcagtcgattcatctaatcaaattttgatatcatattaatatcatattgaataaactc

SEQ ID NO: 11 - DNA, 1000 bp, Gossypium hirsutum
  source 1..1000: mol_type = genomic DNA
acctacttttttttttttcattcaaaagcttatggttcagtagttattgtgtctctcttatgatggggtgtaccaagcttgcaacccacactctttccctttcctcccccagcttgaggactttcgatttttgcaagtgccattttttttaaataaaaaagaaaaagtagatgggaatatatttttttttcacttttataataaagaattttatattatttattaatataggtgggggtagttattctcaaaatacacatctaccacgtattgttgtaacaattttacagaaaaaacaagtatataattattgctatttcaagttatttgattttaagatggtatattttgaaacattgctccatctttattaccattcaaactaatctaattcatatttccattccaaccaacttctccgtttggaaagaatataagatattgcaaaggatcataactaaaataatatttacatttatgttgtcttcttgaattacactatctctttccaatcaggcttggttttagggttgtttgagagtgcaattgtctagggtccacgggtggaaggggctcaaaaaaatttttttttcagcttttaatgtgggaaaaataatttctaaaacaaattaattaaatgtctaaagctttcaatttttcaccataaatgcttcatctcttagggctccaaaattttttcttttattgtattgcattatatatatatattaaaagaagtccaatttattatttcgcctagaacctcaaaaacatcaacaatggacctgaatgttcgactttatcccttaaaccatataacaacaattataaatctataaataaggagttgttacaatgtgaatttcaaaatcctaaaaaaaatataacttttattctcgttaaagactcattatattctagaaatttaattagaatcataacaattgtttcacttatgattctattttgaaatttgttaccgaaatcttaaaaatataat

SEQ ID NO: 12 - DNA, 1000 bp, Gossypium hirsutum
  source 1..1000: mol_type = genomic DNA
ccatacctaatgcctcttcgatttgagatgtttaacaatgaaagtggtacccgatttcaggttaattgacaggaaaaaaaaagcacctcattttaaaacttttttttaccacaatagttacattaatcaagtttttttccactcatgtaacggagaaagcgaaataacacaatatttctcctagtaaaagagatccctattagtgaaaaaagttttcaaagctaatacataaaatttgaatgttttattttttaataaaatgagaaaataatttattttagaagtaaccaaattaatttaatattttctacattaagggataagtagttatctacttacttattattttcaacaatttaaaaaaaaattatattaaataaatagttaatatgattatcaaacaaaattaaatgttgaaaactttcattaaaattttaataatttatgaaacacctaaagctaatatcctgcaatgttttctttttgaagaatttgcactattcaacatcaaatttgctatgtcacacctaaattcataataagtatcaatttcacatgaatgtataaattttttttcacctcaaatgaatttagcagctatatcctcgttgttcaacggataatcctcaatgttgaaacaaccagaaaagaggttgaacatgttggacctaccataaaaaattaaaaatttaattattattataaaatcataagaaatttcaaattattttaaaattataaaaattatattttaaaaaatactaatgacccaaaacatattaaataaataaaaattctctttttttaaaaaataaaaattaaattaatctaatttttcggttttaaatgattcttttgatttttagttttcgagtttttactggttcaaaacggttttaattattttcaattttatgtcactagttggactattattttttttcagtcgattcatctaatcaaattttgatatcatattaatatcatattgaataaactc

SEQ ID NO: 13 - DNA, 2103 bp, Gossypium hirsutum
  source 1..2103: mol_type = genomic DNA
acctacttttttttttttcattcaaaagcttatggttcagtagttattgtgtctctcttatgatggggtgtaccaagcttgcaacccacactctttccctttcctcccccagcttgaggactttcgatttttgcaagtgccattttttttaaataaaaaagaaaaagtagatgggaatatatttttttttcacttttataataaagaattttatattatttattaatataggtgggggtagttattctcaaaatacacatctaccacgtattgttgtaacaattttacagaaaaaacaagtatataattattgctatttcaagttatttgattttaagatggtatattttgaaacattgctccatctttattaccattcaaactaatctaattcatatttccattccaaccaacttctccgtttggaaagaatataagatattgcaaaggatcataactaaaataatatttacatttatgttgtcttcttgaattacactatctctttccaatcaggcttggttttagggttgtttgagagtgcaattgtctagggtccacgggtggaaggggctcaaaaaaatttttttttcagcttttaatgtgggaaaaataatttctaaaacaaattaattaaatgtctaaagctttcaatttttcaccataaatgcttcatctcttagggctccaaaattttttcttttattgtattgcattatatatatatattaaaagaagtccaatttattatttcgcctagaacctcaaaaacatcaacaatggacctgaatgttcgactttatcccttaaaccatataacaacaattataaatctataaataaggagttgttacaatgtgaatttcaaaatcctaaaaaaaatataacttttattctcgttaaagactcattatattctagaaatttaattagaatcataacaattgtttcacttatgattctattttgaaatttgttaccgaaatcttaaaaatataataaaaaaaacattgtgtgtttttttctggaccaatgatagatatatttgtccatagtattaacgacggtaaatctttatcaaattgaacttcataatatactgtccatacctaatgcctcttcgatttgagatgtttaacaatgaaagtggtacccgatttcaggttaattgacaggaaaaaaaaagcacctcattttaaaacttttttttaccacaatagttacattaatcaagtttttttccactcatgtaacggagaaagcgaaataacacaatatttctcctagtaaaagagatccctattagtgaaaaaagttttcaaagctaatacataaaatttgaatgttttattttttaataaaatgagaaaataatttattttagaagtaaccaaattaatttaatattttctacattaagggataagtagttatctacttacttattattttcaacaatttaaaaaaaaattatattaaataaatagttaatatgattatcaaacaaaattaaatgttgaaaactttcattaaaattttaataatttatgaaacacctaaagctaatatcctgcaatgttttctttttgaagaatttgcactattcaacatcaaatttgctatgtcacacctaaattcataataagtatcaatttcacatgaatgtataaattttttttcacctcaaatgaatttagcagctatatcctcgttgttcaacggataatcctcaatgttgaaacaaccagaaaagaggttgaacatgttggacctaccataaaaaattaaaaatttaattattattataaaatcataagaaatttcaaattattttaaaattataaaaattatattttaaaaaatactaatgacccaaaacatattaaataaataaaaattctctttttttaaaaaataaaaattaaattaatctaatttttcggttttaaatgattcttttgatttttagttttcgagtttttactggttcaaaacggttttaattattttcaattttatgtcactagttggactattattttttttcagtcgattcatctaatcaaattttgatatcatattaatatcatattgaataaactc

SEQ ID NO: 14 - DNA, 5000 bp, Gossypium hirsutum
  source 1..5000: mol_type = genomic DNA
agtgcgcacctatgaaggggtacaccctagagttttgtttgagcactaagagtctgtcaagtgtatgaggaagctatgggagacttaactatatttaagatcacaccgaacgtgagtcactaccaagatatggtatgctcgatttgcatgagtattgtccaattagattttgtatcaggtttggttggaagttgttgggattctgtagtaatagaagcggagatcagagtaaaaaagcagagtagcgagatatgtgaaaggtttcttgtacactaaaataagtcaactaaacaaatcatcataagcaacatggatgaccatttctcaacaagagtcaattagattaatttgaccctccccatgactggtcaacagtaaggtaaaccttagccgaatgttcttttacttaccctagaattggtagggaaattagagggaccgagagtatatttatgggaaggggacattttagatggtttttctattttcttaattttcaagttcttctttctaacctcaacatcctcttattcttctttttcgttaagatcagactaagatatcgtctaatgagtagataattttgccttctggtaagtcttatggcatcccacgttacaactataaaagggaagagttttacaggaaggccctgtatggggttacatgtagttgaaatgttagtaaaatgtctgcaatagggattctaatagagatcttatgatcattaagcagttgctgctactagattaagaaagacaatcgaaatagaaacttcaggttgaagtaaaggtagctctctggtgagcctttgaacctgactcctgcatgtcattttatgttgattatgttatgactgatgttcttgtgaacgagtatgtattcttgtagaatgatgtaaaaatgtgtacgtgtgttttctgataagtatctgcaagtacgtaaatgaagtgaacaatgtgcggcatggtatgatgatgtgtgatatacttgtatggggaattatgtgttagaaagtatgattccgttataaatatgaatcaaatttacataagtgtctttgctgtgatgagttggctcatggagtctgggtagatgtgataacttgtattgtaggtgtgagtatggttattgtttaataccttcctattatacaatctcaatactaataagctttatttgtgatgcctctggtatggcagttttattacaaaccagactgatagatcattgttcgaacttatgggtgaatatgataagtgtaagactatgtgggtgaagtcccatgacatcatttggtaatacgccaagatggcgaaacgatgtaaaaccatgtaaggtatagttgattaaggtatcggtgtatattaaatttgcatcatgggactagttagtgaacttataagtaatttggcatcgatgtacagttgattaaggtattatgattcatgtgaattgcatatttggctgaaagtttggtatgaatgtgtaaattagttgatttgaatttgcaggttgatgtttcatttagaaatttgcagaatcagtatgtcacatcatgacatcagacgtttgccatcgtgacgagaccaagttgtcatgccacggtgcgacgtcgagtattgagtgctgcgacgaggcctacctagtatcttgcgtcacaacgaggaaccccttgatgttgcaacgtcaactcgatgctttggaatctttacagttcaattttaattcacctccggttaattttagagctttcgtataagaccaaaaaatgatcgtatattgtattgtatgattgtattgactggaattaatcatttaatggtataagttatttgtaatttgatcgcaattgttccgacaacgaatacaacactttatagcttggactcggtgatcgggtcgtgaacattcaactcaacccttactcagaaaactatgcccaatcctattcgaaaccaaattatcatctattctcaccactgaactcgacttattcccaatgattttaacatttctcttttatagccaatcaagaaggaggccatggatttgaattcaaaactgggtcatagagaaatcaatattcccaattgcaacatcccctaaccatttctgaaactaaatcgtagatcccatcacaaaccagggactttcctcaatagctctcttcatacctttcttactttcttattatcagaagatgctgcaaaaatattttcttcgatttcccttacacagcttcgccattccccttgccaaatacctttgagagtattgagcactcccttccaattcaactcctttgtcaaaattattttacccaccaccatgaacttagtaaaagaaatactattagcatccccatcttcaaacttaattgctaaatcccccattagatctaagaacccaatggagaaaagagtaagattaaagtaatcagcttaaaaaatggcataattagtatttcttacattttgctgatcatgaaacctcaataatggtataacctaggtatcaaaggccaacacacaacataaacaaaccaaaaagacacgaagaaaacctaacaaaaatttaaaaaaaaaatgaaaaagtaaaagaacctgaaaagataaaagagattacctgagatcaaacccaatcaagactcaacaaggaagtgatgacactcatgacgagggaaaaccttatgacgagagactagaagggtttcattttacctaaactaagtaaacattggatttagatgataattaaataatttaaaataatgatacataaaatataaataaatctaatttttgtttatattttaactagttaatataaataagggtattttgattatttttacttccaaacgcaaaagtcaaaagcatctaaatcccaacttttgtgtttgggtgaaaaaaccattttccaacgcagttttgaccccaaaaatcaggcgttcttcattgcttctatggtggaaaagcacttttgaaccttaaaggcaatgaagaacgaggtctatgttgatttatacaatcaaccttactttatatagcatttaataaatttaaaacttactaattaagttttagtttaattggcatccttgctattaaagtaataagaatgtggatcgagtatgtttcaatatgtttttatactgatttaaaggatagagagaagttacgagtaaatataggtattgtataaaaaatatttttctgagttaaagaatattcgtcatagtttgtaaagaggaaaaaaaggaaaaaaaaaaggggaaggaattctatagattaaactttccttataaatatatctcaaagactacatattaacacaatcgacttgtttggttgtaaaaattaacccaacgccttctctagcattattttcaactaattaaaatttatttcttaaaaatggaaattttatatataatatttaaacaaaagataatcacaactttcaattttctttcaaacattttattcctataaaatgaatgccaaacttttttgagttttattttgtgaaatatctttcatataaaaatatttaattattttgaagagaaatatataatttatttaaaatgttttataaaaataaatagacttttattaaatcataaagtaatacttaaacattgatgttattatagctcattttagtagaaatgtagtgaataaacttttccttaggttatttgacactaaccaaataaatgaggcgtaagtgggataagttttaaacgtaataaatttttgtttttttcctaattattaaattgggaaattaatactaattgagtggacattgatcaatattattgagaatgcttcaacttgatcaattggcagccattgctgacctgtgatccttaattttgaaagcgatccaattttattttcattactaattaatactttacttaatgaactactaaatacagcctctcaaaaatccctggctacacattccatttcaattttatattcccacctacttttttttttttcattcaaaagcttatggttcagtagttattgtgtctctcttatgatggggtgtaccaagcttgcaacccacactctttccctttcctcccccagcttgaggactttcgatttttgcaagtgccattttttttaaataaaaaagaaaaagtagatgggaatatatttttttttcacttttataataaagaattttatattatttattaatataggtgggggtagttattctcaaaatacacatctaccacgtattgttgtaacaattttacagaaaaaacaagtatataattattgctatttcaagttatttgattttaagatggtatattttgaaacattgctccatctttattaccattcaaactaatctaattcatatttccattccaaccaacttctccgtttggaaagaatataagatattgcaaaggatcataactaaaataatatttacatttatgttgtcttcttgaattacactatctctttccaatcaggcttggttttagggttgtttgagagtgcaattgtctagggtccacgggtggaaggggctcaaaaaaatttttttttcagcttttaatgtgggaaaaataatttctaaaacaaattaattaaatgtctaaagctttcaatttttcaccataaatgcttcatctcttagggctccaaaattttttcttttattgtattgcattatatatatatattaaaagaagtccaatttattatttcgcctagaacctcaaaaacatcaacaatggacctgaatgttcgactttatcccttaaaccatataacaacaattataaatctataaataaggagttgttacaatgtgaatttcaaaatcctaaaaaaaatataacttttattctcgttaaagactcattatattctagaaatttaattagaatcataacaattgtttcacttatgattctattttgaaatttgttaccgaaatcttaaaaatataat

SEQ ID NO: 15 - DNA, 5000 bp, Gossypium hirsutum
  source 1..5000: mol_type = genomic DNA
ccatacctaatgcctcttcgatttgagatgtttaacaatgaaagtggtacccgatttcaggttaattgacaggaaaaaaaaagcacctcattttaaaacttttttttaccacaatagttacattaatcaagtttttttccactcatgtaacggagaaagcgaaataacacaatatttctcctagtaaaagagatccctattagtgaaaaaagttttcaaagctaatacataaaatttgaatgttttattttttaataaaatgagaaaataatttattttagaagtaaccaaattaatttaatattttctacattaagggataagtagttatctacttacttattattttcaacaatttaaaaaaaaattatattaaataaatagttaatatgattatcaaacaaaattaaatgttgaaaactttcattaaaattttaataatttatgaaacacctaaagctaatatcctgcaatgttttctttttgaagaatttgcactattcaacatcaaatttgctatgtcacacctaaattcataataagtatcaatttcacatgaatgtataaattttttttcacctcaaatgaatttagcagctatatcctcgttgttcaacggataatcctcaatgttgaaacaaccagaaaagaggttgaacatgttggacctaccataaaaaattaaaaatttaattattattataaaatcataagaaatttcaaattattttaaaattataaaaattatattttaaaaaatactaatgacccaaaacatattaaataaataaaaattctctttttttaaaaaataaaaattaaattaatctaatttttcggttttaaatgattcttttgatttttagttttcgagtttttactggttcaaaacggttttaattattttcaattttatgtcactagttggactattattttttttcagtcgattcatctaatcaaattttgatatcatattaatatcatattgaataaactcaaatttacatgctctaaagatggttggttgtgataaataatgttacctaaaagcgacatgtaactgtaactccccatacctagcatagacgacaaggccaagcctagagagttataatgaaagataaccaaccacattctgcaattcataaaaacacacgataaaatctaaataaagacaaatccttatgccgagatgatcatcctattatctgaatttacgaaattaactaacatgatttataatactaaacgataaaagcatataactgagcttcccacccaccgacccgatcaagtctaggatttacctaaaattcattcaacaataactgaattagtatattagaaaggacccaactatttacgtattcaatatattatcggtgtaaccaaatatccaaaagcagatggtgatccagagtcagaaatagttcattcagtatagaccagagttatacagataaatatcagatcaagctataaccaaatgtagaataaacaaagcagatgcagtcatatattaccaactgctacacaccatctccgaccaacccaacacaccaattacggtattcattacccacccaaccctacacaccactcagtgtcagtctgacacgtttcagatattgcagcctagctaccagattagtacactaataccagggattaagcactggttcgacacagaacgcttcctccttattcaattcccacccaatgtatatgcatgtttcatataacagttattacagagttctatgcatacccatgtacagtagatcaattataatgatattagttaaccacaattcacacaataacaatcagtatccaagttctagatcgcaaaatatcaatttaagtagttacattcaatttaacgacatcacaaaaagcttgtattcaattcgaacgatgcttgaaatatagcaaaatttcgcaagctaacctacacgcccatgcctttggcctatgtgtacctaagttataatcagtaggttacatggcctagacacatacctgtgtccttggcccgtgtgaaccctgcactaacatggtcacacacagtctagacacacactcgtgtctatttcccgtgtcactcctattatgtaatcagtgagttacatggcctatcatacggccgtgtggcgtcgacaatcattttaaaatattcttttaaaaaatactttaaccaccaataatttttttgatagtgagcaataatatttcttaacgcatacgttgatactcgaacacaagaccaaagagtagacagattcttaaccagtgaactagcaagctcattcttgaaacagatttacacagaattgagcttaagtcaacaagtcatggatagggttagtttaaaataaagtagcaaaaactttcttaagatgcgactcgaactcctaacctcaaacgcagaaacatagtaaataaccacagatatatatacactcaaatactacaaaattccacaatcaaacattcaaatttttggggcgttacaactctctccccctaaaagaaatttcagccttgaaatttacttgactcaaacaaatacgaatattgctgatgaatcgaatcttcaggtcgtggcttccttagtgtcgtgattctgccaaagaaccttaatcaaatgaataattttcttcctcaaacttaacgtctcgatctaaaatctgaacaggtttctcttgaaaagttacatccaatcaaatttcaatatcctcaataggaacaatgtatgacggatcagatcgatattgtcacaacgtcgaaacgtgaaatacattatgaatttgatccaaattaggaggtaattctaattgatacgcgactgagtttacacgctttaaaatacggtaagggccgataaatctcgagctcaacttgcccttacgaccaaactaaagaacctttttacacagagacacttttaggaacacttgatcgccaacagaaaactcgatatcttttctctttagatccgcataagacttttgttgatcggaagtagtcttaagttgatcttagatcaacttaatagtattctcaatctcaaaaactagctcgagacccaaagcacgaagctcacctaactcggtccaacacagaggagtactacacttatgaccatataaagcctcgtagggagccatctgaatactggattggaaactgttattgtacgtaaattccaacaacggaaagtaatcttcccaactgccctgataatcaatcacacaaccccgaagcatatcctttagaatctgaataacccttttaaactatccatcggtctaagggcgatacacagtactgaactcgagtcaggtgcccaacgcttcatgaagtttcttccaaaattgagaagtaaaacacggatccctatctgagataatcaacgttggaactcaatgcagtcttacaattttagaaacatacagctcggcgagcttctataacgagtaatatgtcctgacagcaaggaaatgagcagacttggtcaatcaattaacgataaaccacaccgaatattttttagtgggtgttaagggtaacccaataacaaaatccatcgtcacccattcccacttccacagagaaattttaattggctgaagtaaccccgaaagcaactggtgttcagccttaaccttttgacacatcaaatagtgtgacacaaaggtacttacctctcgttttagaccaggccaccaatacaatgcttgaagatctcgatacattttattcccaccaagatgcatagcataagggctaatatgtgtctcttgtagaatcaatcatctcaactcctggtcattaggcacagaaattcgatccttgaaatacaaaaccttattgccattcaaactgaaatccgacgattttcccgaatcaagctgacacagtcgctgtaccaaagactcatcacttagctgtttagttcgaatctgatcaacccaagatgtcttaacttaaagttcagccaacaaactcccatcctctaacaaactcaaacgagcaaacatcgttcttagttcagtcatcgctctatgactaagagcatcaactacgacattagctttactaggatggtactcaatagtgcaatcatagtctttaagcaattcaacccacctacacttcctaagatttaactccttttgagttaatagatacttgaggcttttttgatcactgtaaataatacacttttcaccatataggtaatgcttctaaatttttaaagtgaacacgattgcggccagctcaagatcgtgtgtggggtagttaccctcgtgtgtcttaagttatcatgacacttaagctaccactttaccgttttgcatcaaaacacatcctagatcgatgtgcaacgcatcactgaataccacaaactcctggcccaattctggctgagctaaaataggagcttgcgtcaaaacagtcttgagcttttcgaagctagactactgttcctcagaccacacaaatggaatgtttttacgtagtagcttcgttaaaggtgacacaatcaggaagaatccttcaacaaaccttcagtagtaacttgctaaacccaaaaaactctaaagctcaggtacatatttcggctgatttcactcaactatagcttttattttcttgggatcgactcgaatcctctcggcactcacaacgtgccctggaaaagaaacctcctacaaccaaaattcacacttactaaacttaacatacaattgttttttctagagtgtctgaagaacaatacgaaggtgcttattatgttcaaactcagtattgaaataaatcaatatatcatcaataaatactaccacaaattgatcgacatatagttgaaacactagattcattaaatatataaatgcagtcggggcattcgtcaacccaaaaggcatcactaggaattcgtaatgtccataacgagtcctaaaggcagtcttgtacacatcagtctcttt

SEQ ID NO: 16 - DNA, 23 bp, Synthetic construct
  source 1..23: mol_type = other DNA
gaagaagcaccctctcatttacg

SEQ ID NO: 17 - DNA, 19 bp, Synthetic construct
  source 1..19: mol_type = other DNA
tggcagcacagcagatctg

SEQ ID NO: 18 - DNA, 14 bp, Synthetic construct
  source 1..14: mol_type = other DNA
tgcgtccaatgcct

SEQ ID NO: 19 - DNA, 24 bp, Synthetic construct
  source 1..24: mol_type = other DNA
aaatcgaagaggcattaggtatgg

SEQ ID NO: 20 - DNA, 22 bp, Synthetic construct
  source 1..22: mol_type = other DNA
aagagcgaatttggcctgtaga

SEQ ID NO: 21 - DNA, 18 bp, Synthetic construct
  source 1..18: mol_type = other DNA
attgcgagctttctaatt

SEQ ID NO: 22 - DNA, 31 bp, Synthetic construct
  source 1..31: mol_type = other DNA
ctggaccaatgatagatatatttgtccatag

SEQ ID NO: 23 - DNA, 21 bp, Synthetic construct
  source 1..21: mol_type = other DNA
cgacggtaaatctttatcaaa

SEQ ID NO: 24 - DNA, 442 bp, unidentified
  source 1..442: mol_type = other DNA; note = Agrobacterium tumefaciens
ctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggactttt

SEQ ID NO: 25 - DNA, 34 bp, unidentified
  source 1..34: mol_type = other DNA; note = Escherichia virus P1
ataacttcgtatagcatacattatacgaagttat

SEQ ID NO: 26 - DNA, 500 bp, Medicago truncatula
  source 1..500: mol_type = genomic DNA
acacatattcaagctctattactctgtttttaggttgagaatattgtgaagtgtgttgtgtgtgcgttctgttttatgtaaaagtaaattctgtgaattatattttgtaaggaataaattatgtaagaatggtttaattatctattgtctcacatcgttttttatgtctattttaagtattggtattgtattctatttatacacaagagttaataatagcataacataaattccacaaggatcaagtaaaataatatccacatgaaacatctaagaaaggaaaacaatatggctcaatttcctatctttctttatttatgttttagtcatctttctttccctatttcttgttgaaagctaataggagtatggtactttctacgccatttttttttaagtactctcttttacacaatattttatcaaattgtagtgacattttattttacgatgcagttaatgttcgttgtgttactgtcaatgattgtccaaaggtgaaa

SEQ ID NO: 27 - DNA, 552 bp, Synthetic construct
  source 1..552: mol_type = other DNA
atgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttga

SEQ ID NO: 28 - DNA, 203 bp, Arabidopsis thaliana
  source 1..203: mol_type = genomic DNA
aacttggccaatccgcatcgtgtggcgatcagagagttaagccttgaagacgaagagataacgaattggttgttgatcactcgctttataaatctctcagtttcttgctcacaccaacatctctctaagcttcttcttctaccaatctaattcctctcttcagcttcttgtgttgtgacgcatactcgtcgcagtcttgagat

SEQ ID NO: 29 - DNA, 1421 bp, Arabidopsis thaliana
  source 1..1421: mol_type = genomic DNA
agttctaatgtgttactgtatcaaaaaaaatttggtattgttttctagacatgatataattttgtacttgtaattgattttagaaagatattttctataatgaaaaaaatcttataaaactttttaaaaagttaccaaattaggaaaatttctctataattaaaaaatattaatttatcgataaattaaaacctttttaaattaataaaattttgcagtcccaacattattaatttataaaggttttactgtacttcattttctaacctcagtatgttaaaagtcatgtatgtgtactaagaatcaaacctggcacgcaaacttttgctaattttgtcacatacagttaacatttagcattgtgcgagtagctccaccacattattatcaatcgtttggttcttcaacatgtcttatatatggatcaataatagtttaatatcatatcagtaatctttgttctcttgtcttgcatgtagaacatgtacatattgcttcgtcggcagcgaatgaagcctaaaattaaaatttggttttgtccattgatgatatgatggctcgttccacatgagagaatggttgagaaaactgaaaataatgaagaggatgagaaattataatgatcagacaagtgaagtgtttcattttatagcttagtgtagaaaacgattgcttgtgattgtgaatatatagttttagatgagttgtttttatagtaactttgagggaacaataaactatgatatagttatcatttttattttaaaacaagtaaattcatatgaatttctgatatattcatattttcactatgaatttaaacactataatcttatttcatattttagcatatgatattttgcatgatgctatcttagcctcgactatctttacttcatcttctaaactatatatttatcaaatcttacgtatgcattgcatcgcttgtttttacattttgtgtaaagaaaaagttgcattaattagtttttagcggtttagtacaatatggttaatttgataaccacatacttgatacctgttgtatctagagttgtatataacaaaattagaattatattctattggctaagccaatttctctccaccaatcagtttacatcggttatttggaacattattatcgataagcaatgtcattatccaaaattttggaatgttatcgtatgaagaagagtgtagaagtatcttgatcacatatgaatggcttacctcaatcctcaaaaaaaaaaaaaaacaatgattttccaaagcatatgtggtaataaataatgttataatcttcacttgaaaaaaaaaacaaatccattggcacagattagccatttgacgacaacctcataaacatattccacgtggaccaatcaaaatagaatcctcctaatcatcttcctcgtggc

SEQ ID NO: 30 - DNA, 304 bp, Strawberry vein banding virus
  source 1..304: mol_type = genomic DNA
gaagcccgctttacaagtggccagctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattatt

SEQ ID NO: 31 - DNA, 1086 bp, Cucumis melo
  source 1..1086: mol_type = genomic DNA
tttgtatctattttatctattaaaggtatgtttgggcttcaggttggaatggagtagaattgaatgggttggggagtaaattttccattcaacaagttcaatttcaaaatggctaataagttttgaactcaattttattttcaataaattccttaattttttgttccttgtttgtaaactattgacttattcgatatattttaaaattgaggtattttaaaaaaataatacaatattaaaattatttataaaatataacaaaatttatgtatagtttatttgaaaattttactatagtttcatttttatattattcctaaccatttccatttaaaattatttcaattatttcttttattaatataattgaaatttcatggatttattagacacatgatttgaaattttatgggtttattaagtattttctaacacaaaatcgcttccgcatcgttttcaattcattcagtaatagaagtaattttttaaaagaaccaaatttgccaaattttgagttccataaggactctgaaaactcattatgtctattactcttcactaattgtagagacttaaattcaagaaaagagacactaattgatgataattgcccaaaaaataaaaataaaaatgtttcttccccatcctcaacctccatgaattcacagagcccaaagattaattattgggccccaattcctactcatatataccttacagtccctcaaagaaatcttaggaagtaatcaatttctgtttattcaagatgtagcctcccaaaagaaaaatacatcacatcaaattcaaacaaaaatatctacagctagcaaaacctcaaaccgttaaaatttcaagccacataaatgaaattttcatctgaaaaaaggacaatctatctagacgttagatttcagccctaatatgaatctgaagcatttggtggacgagaaagagccatgtaggaatgcatcaaacaaaggaaaaatctttgaactccaatgggattgaagatacagataccaatggataagaatctgttctctttgcccactatttaaactcaccaaacccaccagtatcttcctc

SEQ ID NO: 32 - DNA, 54 bp, Cucumis melo
  source 1..54: mol_type = genomic DNA
accacaaaatacattccaccgttgatcacaagccttattccaccacctccaaca

SEQ ID NO: 33 - DNA, 1056 bp, Synthetic construct
  source 1..1056: mol_type = other DNA
atggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctga

SEQ ID NO: 34 - DNA, 500 bp, Medicago truncatula
  source 1..500: mol_type = genomic DNA
aagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctc

SEQ ID NO: 35 - DNA, 829 bp, Arabidopsis thaliana
  source 1..829: mol_type = genomic DNA
gttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttctt

SEQ ID NO: 36 - DNA, 67 bp, Arabidopsis thaliana
  source 1..67: mol_type = genomic DNA
cacaattcagatttcaatttctcaaaatcttaaaaactttctctcaattctctctaccgtgatcaag

SEQ ID NO: 37 - DNA, 306 bp, Arabidopsis thaliana
  source 1..306: mol_type = genomic DNA
gtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggt

SEQ ID NO: 38 - DNA, 204 bp, Arabidopsis thaliana
  source 1..204: mol_type = genomic DNA
atggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgt

SEQ ID NO: 39 - DNA, 1023 bp, Synthetic construct
  source 1..1023: mol_type = other DNA
atgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtga

SEQ ID NO: 40 - DNA, 500 bp, Medicago truncatula
  source 1..500: mol_type = genomic DNA
gagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagtta

SEQ ID NO: 41 - DNA, 537 bp, Figwort mosaic virus
  source 1..537: mol_type = genomic DNA
aattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaag

SEQ ID NO: 42 - DNA, 480 bp, Arabidopsis thaliana
  source 1..480: mol_type = genomic DNA
ggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattcc

SEQ ID NO: 43 - DNA, 46 bp, Arabidopsis thaliana
  source 1..46: mol_type = genomic DNA
atttatcgttcttattgaccctagccgctacacacttttctgcgat

SEQ ID NO: 44 - DNA, 622 bp, Arabidopsis thaliana
  source 1..622: mol_type = genomic DNA
atctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccag

SEQ ID NO: 45 - DNA, 210 bp, Synthetic construct
  source 1..210: mol_type = other DNA
atggcggcactggtgacctcccagctcgcgacaagcggcaccgtcctgtcggtgacggaccgcttccggcgtcccggcttccagggactgaggccacggaacccagccgatgccgctctcgggatgaggacggtgggcgcgtccgcggctcccaagcagagcaggaagccacaccgtttcgaccgccggtgcttgagcatggtcgtctgc

SEQ ID NO: 46 - DNA, 1368 bp, Synthetic construct
  source 1..1368: mol_type = other DNA
atgcttcacggtgcttcttccagacccgcaacggctagaaagagttctggcttgtctggaaccgtccgtattccaggagacaaaagcattagtcaccgctctttcatgtttggtgggctggcatctggagagacgcgcatcactggtcttctggaaggagaggacgtcatcaatacagggaaggcaatgcaggctatgggtgcccgtattcgcaaggaaggtgatacttggatcatagacggagttgggaacggtggcttacttgcaccggaggctcctctcgactttggcaacgcagccacagggtgtagacttactatgggcctcgtgggtgtttacgatttcgattcaacctttattggggatgcctctctcactaaacgcccaatgggaagagtccttaacccgttgagggagatgggcgtacaagttaagtccgaggacggcgacagattgcccgtcaccttgcgcggccctaagacacccacccctattacttacagggttccaatggcatctgctcaagtgaagtccgcagttctgctcgctggattgaacacaccgggtattactaccgtgattgagccgatcatgactcgtgaccacactgagaagatgcttcagggtttcggtgctaacctcaccgttgaaacagacgcggacggtgtgaggaccattcgcctggagggaaggggaaaactcactggtcaagtcattgacgtgcccggtgatccctccagcacggcgttcccactggttgccgctcttctcgtaccaggctccgatgtgacaattctaaacgtcctcatgaatcctactagaaccggattgatacttacattgcaggaaatgggtgctgatattgaagttatcaatcctagactagccggaggtgaggacgtagctgatttgcgggtgaggtcttctacattgaaaggtgttaccgtacctgaagatagggcaccttcaatgattgacgagtatccaattcttgccgtcgcggctgcctttgctgagggcgcgaccgtgatgaatggactagaggagttgagagtgaaggaatccgacagattgagcgcagtcgctaacggacttaaactcaatggcgttgattgtgatgagggtgagactagcttggtagtccgtgggcgaccagacggaaagggtttgggcaacgcttcgggtgctgccgttgcaactcacttggatcatcggatagcgatgagttttctggtgatgggtctcgtaagcgagaatcctgtgacagtcgacgatgcaactatgatcgctacttccttccctgagtttatggatttaatggcaggactaggtgcaaagattgaactctctgataccaaagcggcctaa

SEQ ID NO: 47 - DNA, 643 bp, Pisum sativum
  source 1..643: mol_type = genomic DNA
agctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaat

SEQ ID NO: 48 - DNA, 1024 bp, Synthetic construct
  source 1..1024: mol_type = other DNA
ctagctaactaagcttcctcccaggttttcgggactcgccagacctaaattattcccgtttgttgccgctaccgggcctcggtggggccctccctggcactccgggctgagcgcacgcgtaattacgccgacctagcgattggtgaacccgacaagaaggtgcgggcctagttggcgggcttggggcctacccgcgtgggctgaacagaaggaaccctcaagtgaaagagtttgaaactgggcgtcacctcggactaattactcgcgtggcacgtgctccgggtcggccctgaggctgtaggcgcaaagcgaaagtccaggataggggagaggacggccaacccgcctcggtatcctggattcctaacctctgaccggatcagcaatatcgctccacactaaccgcccccttttccgactccgggtgctcttcgcggagttggctagagacaagtgagcacggatcagacgcgagagaggcaatcgtttatttgtaagcgcctactgccccaaccgatcgcggtgtgcagagaggattgtgctatactgaagcgggtgtgtcactaagggttgcggtgggaagcccgggcagtgactaagcctgatccgagcccccaggcactactccgaccttttagcacgttgtgacggtctgccaagggttctcacgttaatttagcgatcggggtaacaagggccagaacggtcccccctgtatcaccgctctgcagtggagttttccccagcgtcctcaaatcgtgggactcggcaggcggtcactgcccagcccgtgtataaggtagggctcccgtactgtacgaaataatatccctgtaactctgttcgccccagcgttgagttagcacgcacttatttcactgagcgcctgcgagagcgccgaggggttattgagactagcgataagctggaagtccgacctgcgggggccaaccgtaccgccactcacggggcgtcttgaacgtccgtaccccgactccgcagattaagtgcactagttagttaa

SEQ ID NO: 49 - DNA, 315 bp, Gossypium barbadense
  source 1..315: mol_type = genomic DNA
accatatgacactggtgcatgtgccatcatcatgcagtaatttcatggtatatcttaattatatggttaataaaaaaaagatggtgagtgaataatgtgcgtgcattcctccatgcaccaatggtgaatctctttgcatacatagagattctgaatgattatagtttatgttgtagtgaaattaattttgaatgttgtttttaaattttaatgtcacttggcttgatttatgttttaacgaagcttatgttatgtattttactttaatgatattgcatgtattgttaatttaacattgcttgatcagtatactct

SEQ ID NO: 50 - DNA, 540 bp, Synthetic construct
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 51 - DNA, 204 bp, Synthetic construct
  source 1..204: mol_type = other DNA
atggccaccgccaccactaccgccaccgctgcgttctccggcgtggtgagcgtcggcactgagacgcgcaggatctactccttcagccacctccagccttctgctgcgttccccgctaagccgtcttcgttcaagagcctgaagctgaaacagtccgcacgccttacccggcgcctggaccataggccattcgttgtcaggtgc

SEQ ID NO: 52 - DNA, 348 bp, Arabidopsis thaliana
  source 1..348: mol_type = genomic DNA
caggtaatttctctcctctctatttttaccattttccattgacgacgatctaggttttctgatttgattttggagaacgcctcgatgagtttatagattcgtagattggttttgagattcagtataatttcacccggattccaatttttgaaccgatacctaattttgaattgatttggtagatcgattggtcaaatttgaaattgatttttctccataatatctgaagcgtcttattggatcaaatctacaacatttctctgttgaaaggatcgattttttttttcttggaacatgataacttttgattattcatcaaagttttgttctttttaatatttcacaggt

SEQ ID NO: 53 - DNA, 72 bp, Arabidopsis thaliana
  source 1..72: mol_type = genomic DNA
agtgagtcacataaccctcttggaaagagtctcaacacttgcagagaaaaagaacaaggaagatcccggaaa

SEQ ID NO: 54 - DNA, 20 bp, Synthetic construct
  source 1..20: mol_type = other DNA
ctcaactctcagctaaacca

SEQ ID NO: 55 - DNA, 480 bp, Synthetic construct
  source 1..480: mol_type = other DNA
tgttaatgttatccgaactagtcataattacaaccgacaaaataaggttattttgtgtgttatagaattttttggacagtttttgttttggttttcgattgtagtaaaaatagatttatgtaataagatttacttttcttgttgaaacaaaataatcttagaattaactcaacttttatgttagaacaaatgataaaaaaatttccccttttctatgcgattattttcaatcagagagaaatacatataatatatataattcaaattaatctgccaaattaataaatttggattaaaatttataaatgaaacaatggtgtaaggcaattaaaaacacaacactaaaaatatgagaacattttatctgggcattaagagtttgggctttagatctaaaataaaggccggcccaacgagaatattaaaccctaattgacctagttccctatatatataaaccctatatttctctcgtcactc

SEQ ID NO: 56 - DNA, 422 bp, Synthetic construct
  source 1..422: mol_type = other DNA
agtacaatcatacaagagcaatatatatatttttggttattgaaatttaaatatcatcttcacaaaatgaaaaagcacaaaaagtattaattaatatcatgttttgagactcctttttaccaagaatataaatttcacacctaagaaaattctgaactaggaaaataaccagcatacaattaaggaataagaaaatgcaattacgataaacacttgtcacaaattgtttaataagtcactatccaatcaattatcaaaagtaagatattgccacgtggcaaccagtattttcatcaccttatcaaaagataagcaaaagaaccacatcaaagccacaaaatgccaaccacagatggataaggaaaatccaaccaaccacatgtaatcccacacctcatcaccttatccacacctctgtctat

SEQ ID NO: 57 - DNA, 505 bp, unidentified
  source 1..505: mol_type = other DNA; note = Agrobacterium tumefaciens
cgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcctt

SEQ ID NO: 58 - DNA, 50 bp, Gossypium hirsutum
  source 1..50: mol_type = genomic DNA
acctacttttttttttttcattcaaaagcttatggttcagtagttattgt

SEQ ID NO: 59 - DNA, 50 bp, Gossypium hirsutum
  source 1..50: mol_type = genomic DNA
gtctctcttatgatggggtgtaccaagcttgcaacccacactctttccct

SEQ ID NO: 60 - DNA, 50 bp, Gossypium hirsutum
  source 1..50: mol_type = genomic DNA
ttcctcccccagcttgaggactttcgatttttgcaagtgccatttttttt

SEQ ID NO: 61 - DNA, 50 bp, Gossypium hirsutum
  source 1..50: mol_type = genomic DNA
aaataaaaaagaaaaagtagatgggaatatatttttttttcacttttata

SEQ ID NO: 62 - DNA, 50 bp, Gossypium hirsutum
  source 1..50: mol_type = genomic DNA
ataaagaattttatattatttattaatataggtgggggtagttattctca

SEQ ID NO: 63 - DNA, 50 bp, Gossypium hirsutum
  source 1..50: mol_type = genomic DNA
aaatacacatctaccacgtattgttgtaacaattttacagaaaaaacaag

SEQ ID NO: 64 - DNA, 50 bp, Gossypium hirsutum
  source 1..50: mol_type = genomic DNA
tatataattattgctatttcaagttatttgattttaagatggtatatttt

SEQ ID NO: 65 - DNA, 50 bp, Gossypium hirsutum
  source 1..50: mol_type = genomic DNA
gaaacattgctccatctttattaccattcaaactaatctaattcatattt

SEQ ID NO: 66 - DNA, 50 bp, Gossypium hirsutum
  source 1..50: mol_type = genomic DNA
ccattccaaccaacttctccgtttggaaagaatataagatattgcaaagg

SEQ ID NO: 67 - DNA, 50 bp, Gossypium hirsutum
  source 1..50: mol_type = genomic DNA
atcataactaaaataatatttacatttatgttgtcttcttgaattacact

SEQ ID NO: 68 - DNA, 50 bp, Gossypium hirsutum
  source 1..50: mol_type = genomic DNA
atctctttccaatcaggcttggttttagggttgtttgagagtgcaattgt

SEQ ID NO: 69 - DNA, 50 bp, Gossypium hirsutum
  source 1..50: mol_type = genomic DNA
ctagggtccacgggtggaaggggctcaaaaaaatttttttttcagctttt

SEQ ID NO: 70 - DNA, 50 bp, Gossypium hirsutum
  source 1..50: mol_type = genomic DNA
aatgtgggaaaaataatttctaaaacaaattaattaaatgtctaaagctt

SEQ ID NO: 71 - DNA, 50 bp, Gossypium hirsutum
  source 1..50: mol_type = genomic DNA
tcaatttttcaccataaatgcttcatctcttagggctccaaaattttttc

SEQ ID NO: 72 - DNA, 50 bp, Gossypium hirsutum
  source 1..50: mol_type = genomic DNA
ttttattgtattgcattatatatatatattaaaagaagtccaatttatta

SEQ ID NO: 73 - DNA, 50 bp, Gossypium hirsutum
  source 1..50: mol_type = genomic DNA
tttcgcctagaacctcaaaaacatcaacaatggacctgaatgttcgactt

SEQ ID NO: 74 - DNA, 50 bp, Gossypium hirsutum
  source 1..50: mol_type = genomic DNA
tatcccttaaaccatataacaacaattataaatctataaataaggagttg

SEQ ID NO: 75 - DNA, 50 bp, Gossypium hirsutum
  source 1..50: mol_type = genomic DNA
ttacaatgtgaatttcaaaatcctaaaaaaaatataacttttattctcgt

SEQ ID NO: 76 - DNA, 50 bp, Gossypium hirsutum
  source 1..50: mol_type = genomic DNA
taaagactcattatattctagaaatttaattagaatcataacaattgttt

SEQ ID NO: 77 - DNA, 50 bp, Gossypium hirsutum
  source 1..50: mol_type = genomic DNA
cacttatgattctattttgaaatttgttaccgaaatcttaaaaatataat

SEQ ID NO: 78 - DNA, 50 bp, Gossypium hirsutum
  source 1..50: mol_type = genomic DNA
agtgcgcacctatgaaggggtacaccctagagttttgtttgagcactaag

SEQ ID NO: 79 - DNA, 50 bp, Gossypium hirsutum
  source 1..50: mol_type = genomic DNA
tcacaccgaacgtgagtcactaccaagatatggtatgctcgatttgcatg

SEQ ID NO: 80 - DNA, 50 bp, Gossypium hirsutum
  source 1..50: mol_type = genomic DNA
ttctgtagtaatagaagcggagatcagagtaaaaaagcagagtagcgaga

SEQ ID NO: 81 - DNA, 50 bp, Gossypium hirsutum
  source 1..50: mol_type = genomic DNA
ataagcaacatggatgaccatttctcaacaagagtcaattagattaattt

SEQ ID NO: 82 - DNA, 50 bp, Gossypium hirsutum
  source 1..50: mol_type = genomic DNA
ttttacttaccctagaattggtagggaaattagagggaccgagagtatat

SEQ ID NO: 83 - DNA, 50 bp, Gossypium hirsutum
  source 1..50: mol_type = genomic DNA
gttcttctttctaacctcaacatcctcttattcttctttttcgttaagat

SEQ ID NO: 84 - DNA, 50 bp, Gossypium hirsutum
  source 1..50: mol_type = genomic DNA
ttatggcatcccacgttacaactataaaagggaagagttttacaggaagg

SEQ ID NO: 85 - DNA, 50 bp, Gossypium hirsutum
  source 1..50: mol_type = genomic DNA
gggattctaatagagatcttatgatcattaagcagttgctgctactagat

SEQ ID NO: 86 - DNA, 50 bp, Gossypium hirsutum
  source 1..50: mol_type = genomic DNA
ctggtgagcctttgaacctgactcctgcatgtcattttatgttgattatg

SEQ ID NO: 87 - DNA, 50 bp, Gossypium hirsutum
  source 1..50: mol_type = genomic DNA
aaaatgtgtacgtgtgttttctgataagtatctgcaagtacgtaaatgaa

SEQ ID NO: 88 - DNA, 50 bp, Gossypium hirsutum
  source 1..50: mol_type = genomic DNA
aattatgtgttagaaagtatgattccgttataaatatgaatcaaatttac

SEQ ID NO: 89 - DNA, 50 bp, Gossypium hirsutum
  source 1..50: mol_type = genomic DNA
ataacttgtattgtaggtgtgagtatggttattgtttaataccttcctat

SEQ ID NO: 90 - DNA, 50 bp, Gossypium hirsutum
  source 1..50: mol_type = genomic DNA
agttttattacaaaccagactgatagatcattgttcgaacttatgggtga

SEQ ID NO: 91 - DNA, 50 bp, Gossypium hirsutum
  source 1..50: mol_type = genomic DNA
aatacgccaagatggcgaaacgatgtaaaaccatgtaaggtatagttgat

SEQ ID NO: 92 - DNA, 50 bp, Gossypium hirsutum
  source 1..50: mol_type = genomic DNA
tataagtaatttggcatcgatgtacagttgattaaggtattatgattcat

SEQ ID NO: 93 - DNA, 50 bp, Gossypium hirsutum
  source 1..50: mol_type = genomic DNA
atttgaatttgcaggttgatgtttcatttagaaatttgcagaatcagtat

SEQ ID NO: 94 - DNA, 50 bp, Gossypium hirsutum
  source 1..50: mol_type = genomic DNA
tgccacggtgcgacgtcgagtattgagtgctgcgacgaggcctacctagt

SEQ ID NO: 95 - DNA, 50 bp, Gossypium hirsutum
  source 1..50: mol_type = genomic DNA
ctttggaatctttacagttcaattttaattcacctccggttaattttaga

SEQ ID NO: 96 - DNA, 50 bp, Gossypium hirsutum
  source 1..50: mol_type = genomic DNA
ttgactggaattaatcatttaatggtataagttatttgtaatttgatcgc

SEQ ID NO: 97 - DNA, 50 bp, Gossypium hirsutum
  source 1..50: mol_type = genomic DNA
ggtcgtgaacattcaactcaacccttactcagaaaactatgcccaatcct

SEQ ID NO: 98 - DNA, 50 bp, Gossypium hirsutum
  source 1..50: mol_type = genomic DNA
caatgattttaacatttctcttttatagccaatcaagaaggaggccatgg

SEQ ID NO: 99 - DNA, 50 bp, Gossypium hirsutum
  source 1..50: mol_type = genomic DNA
atcccctaaccatttctgaaactaaatcgtagatcccatcacaaaccagg

SEQ ID NO: 100 - DNA, 50 bp, Gossypium hirsutum
  source 1..50: mol_type = genomic DNA
aagatgctgcaaaaatattttcttcgatttcccttacacagcttcgccat

SEQ ID NO: 101 - DNA, 50 bp, Gossypium hirsutum
  source 1..50: mol_type = genomic DNA
ctcctttgtcaaaattattttacccaccaccatgaacttagtaaaagaaa

SEQ ID NO: 102 - DNA, 50 bp, Gossypium hirsutum
  source 1..50: mol_type = genomic DNA
tctaagaacccaatggagaaaagagtaagattaaagtaatcagcttaaaa

SEQ ID NO: 103 - DNA, 50 bp, Gossypium hirsutum
  source 1..50: mol_type = genomic DNA
atggtataacctaggtatcaaaggccaacacacaacataaacaaaccaaa

SEQ ID NO: 104 - DNA, 50 bp, Gossypium hirsutum
  source 1..50: mol_type = genomic DNA
aagaacctgaaaagataaaagagattacctgagatcaaacccaatcaaga

SEQ ID NO: 105 - DNA, 50 bp, Gossypium hirsutum
  source 1..50: mol_type = genomic DNA
agactagaagggtttcattttacctaaactaagtaaacattggatttaga

SEQ ID NO: 106 - DNA, 50 bp, Gossypium hirsutum
  source 1..50: mol_type = genomic DNA
atttttgtttatattttaactagttaatataaataagggtattttgatta

SEQ ID NO: 107 - DNA, 50 bp, Gossypium hirsutum
  source 1..50: mol_type = genomic DNA
gtttgggtgaaaaaaccattttccaacgcagttttgaccccaaaaatcag

SEQ ID NO: 108 - DNA, 50 bp, Gossypium hirsutum
  source 1..50: mol_type = genomic DNA
aatgaagaacgaggtctatgttgatttatacaatcaaccttactttatat

SEQ ID NO: 109 - DNA, 50 bp, Gossypium hirsutum
  source 1..50: mol_type = genomic DNA
tccttgctattaaagtaataagaatgtggatcgagtatgtttcaatatgt

SEQ ID NO: 110 - DNA, 50 bp, Gossypium hirsutum
  source 1..50: mol_type = genomic DNA
tgtataaaaaatatttttctgagttaaagaatattcgtcatagtttgtaa

SEQ ID NO: 111 - DNA, 50 bp, Gossypium hirsutum
  source 1..50: mol_type = genomic DNA
ttccttataaatatatctcaaagactacatattaacacaatcgacttgtt

SEQ ID NO: 112 - DNA, 50 bp, Gossypium hirsutum
  source 1..50: mol_type = genomic DNA
taaaatttatttcttaaaaatggaaattttatatataatatttaaacaaa

SEQ ID NO: 113 - DNA, 50 bp, Gossypium hirsutum
  source 1..50: mol_type = genomic DNA
gaatgccaaacttttttgagttttattttgtgaaatatctttcatataaa

SEQ ID NO: 114 - DNA, 50 bp, Gossypium hirsutum
  source 1..50: mol_type = genomic DNA
taaaaataaatagacttttattaaatcataaagtaatacttaaacattga

SEQ ID NO: 115 - DNA, 50 bp, Gossypium hirsutum
  source 1..50: mol_type = genomic DNA
ggttatttgacactaaccaaataaatgaggcgtaagtgggataagtttta

SEQ ID NO: 116 - DNA, 50 bp, Gossypium hirsutum
  source 1..50: mol_type = genomic DNA
actaattgagtggacattgatcaatattattgagaatgcttcaacttgat

SEQ ID NO: 117 - DNA, 50 bp, Gossypium hirsutum
  source 1..50: mol_type = genomic DNA
attttattttcattactaattaatactttacttaatgaactactaaatac

SEQ ID NO: 118 - DNA, 50 bp, Gossypium hirsutum; identical to SEQ ID NO: 58
  source 1..50: mol_type = genomic DNA

SEQ ID NO: 119 - DNA, 50 bp, Gossypium hirsutum; identical to SEQ ID NO: 60
  source 1..50: mol_type = genomic DNA

SEQ ID NO: 120 - DNA, 50 bp, Gossypium hirsutum; identical to SEQ ID NO: 62
  source 1..50: mol_type = genomic DNA

SEQ ID NO: 121 - DNA, 50 bp, Gossypium hirsutum; identical to SEQ ID NO: 64
  source 1..50: mol_type = genomic DNA

SEQ ID NO: 122 - DNA, 50 bp, Gossypium hirsutum; identical to SEQ ID NO: 66
  source 1..50: mol_type = genomic DNA

SEQ ID NO: 123 - DNA, 50 bp, Gossypium hirsutum; identical to SEQ ID NO: 68
  source 1..50: mol_type = genomic DNA

SEQ ID NO: 124 - DNA, 50 bp, Gossypium hirsutum; identical to SEQ ID NO: 70
  source 1..50: mol_type = genomic DNA

SEQ ID NO: 125 - DNA, 50 bp, Gossypium hirsutum; identical to SEQ ID NO: 72
  source 1..50: mol_type = genomic DNA

SEQ ID NO: 126 - DNA, 50 bp, Gossypium hirsutum; identical to SEQ ID NO: 74
  source 1..50: mol_type = genomic DNA

SEQ ID NO: 127 - DNA, 50 bp, Gossypium hirsutum; identical to SEQ ID NO: 76
  source 1..50: mol_type = genomic DNA

SEQ ID NO: 128 - DNA, 50 bp, Gossypium hirsutum
  source 1..50: mol_type = genomic DNA
ccatacctaatgcctcttcgatttgagatgtttaacaatgaaagtggtac

SEQ ID NO: 129 - DNA, 50 bp, Gossypium hirsutum
  source 1..50: mol_type = genomic DNA
ccgatttcaggttaattgacaggaaaaaaaaagcacctcattttaaaact

SEQ ID NO: 130 - DNA, 50 bp, Gossypium hirsutum
  source 1..50: mol_type = genomic DNA
tttttttaccacaatagttacattaatcaagtttttttccactcatgtaa

SEQ ID NO: 131 - DNA, 50 bp, Gossypium hirsutum
  source 1..50: mol_type = genomic DNA
cggagaaagcgaaataacacaatatttctcctagtaaaagagatccctat

SEQ ID NO: 132 - DNA, 50 bp, Gossypium hirsutum
  source 1..50: mol_type = genomic DNA
tagtgaaaaaagttttcaaagctaatacataaaatttgaatgttttattt

SEQ ID NO: 133 - DNA, 50 bp, Gossypium hirsutum
  source 1..50: mol_type = genomic DNA
tttaataaaatgagaaaataatttattttagaagtaaccaaattaattta

SEQ ID NO: 134 - DNA, 50 bp, Gossypium hirsutum
  source 1..50: mol_type = genomic DNA
atattttctacattaagggataagtagttatctacttacttattattttc

SEQ ID NO: 135 - DNA, 50 bp, Gossypium hirsutum
  source 1..50: mol_type = genomic DNA
aacaatttaaaaaaaaattatattaaataaatagttaatatgattatcaa

SEQ ID NO: 136 - DNA, 50 bp, Gossypium hirsutum
  source 1..50: mol_type = genomic DNA
acaaaattaaatgttgaaaactttcattaaaattttaataatttatgaaa

SEQ ID NO: 137 - DNA, 50 bp, Gossypium hirsutum
  source 1..50: mol_type = genomic DNA
cacctaaagctaatatcctgcaatgttttctttttgaagaatttgcacta

SEQ ID NO: 138 - DNA, 50 bp, Gossypium hirsutum
  source 1..50: mol_type = genomic DNA
ttcaacatcaaatttgctatgtcacacctaaattcataataagtatcaat

SEQ ID NO: 139 - DNA, 50 bp, Gossypium hirsutum
  source 1..50: mol_type = genomic DNA
ttcacatgaatgtataaattttttttcacctcaaatgaatttagcagcta

SEQ ID NO: 140 - DNA, 50 bp, Gossypium hirsutum
  source 1..50: mol_type = genomic DNA
tatcctcgttgttcaacggataatcctcaatgttgaaacaaccagaaaag

SEQ ID NO: 141 - DNA, 50 bp, Gossypium hirsutum
  source 1..50: mol_type = genomic DNA
aggttgaacatgttggacctaccataaaaaattaaaaatttaattattat

SEQ ID NO: 142 - DNA, 50 bp, Gossypium hirsutum
  source 1..50: mol_type = genomic DNA
tataaaatcataagaaatttcaaattattttaaaattataaaaattatat

SEQ ID NO: 143 - DNA, 50 bp, Gossypium hirsutum
  source 1..50: mol_type = genomic DNA
tttaaaaaatactaatgacccaaaacatattaaataaataaaaattctct

SEQ ID NO: 144 - DNA, 50 bp, Gossypium hirsutum
  source 1..50: mol_type = genomic DNA
ttttttaaaaaataaaaattaaattaatctaatttttcggttttaaatga

SEQ ID NO: 145 - DNA, 50 bp, Gossypium hirsutum
  source 1..50: mol_type = genomic DNA
ttcttttgatttttagttttcgagtttttactggttcaaaacggttttaa

SEQ ID NO: 146 - DNA, 50 bp, Gossypium hirsutum
  source 1..50: mol_type = genomic DNA
ttattttcaattttatgtcactagttggactattattttttttcagtcga

SEQ ID NO: 147 - DNA, 50 bp, Gossypium hirsutum
  source 1..50: mol_type = genomic DNA
ttcatctaatcaaattttgatatcatattaatatcatattgaataaactc

SEQ ID NO: 148 - DNA, 50 bp, Gossypium hirsutum; identical to SEQ ID NO: 129
  source 1..50: mol_type = genomic DNA

SEQ ID NO: 149 - DNA, 50 bp, Gossypium hirsutum; identical to SEQ ID NO: 131
  source 1..50: mol_type = genomic DNA

SEQ ID NO: 150 - DNA, 50 bp, Gossypium hirsutum; identical to SEQ ID NO: 133
  source 1..50: mol_type = genomic DNA

SEQ ID NO: 151 - DNA, 50 bp, Gossypium hirsutum; identical to SEQ ID NO: 135
  source 1..50: mol_type = genomic DNA

SEQ ID NO: 152 - DNA, 50 bp, Gossypium hirsutum; identical to SEQ ID NO: 137
  source 1..50: mol_type = genomic DNA

SEQ ID NO: 153 - DNA, 50 bp, Gossypium hirsutum; identical to SEQ ID NO: 139
  source 1..50: mol_type = genomic DNA

SEQ ID NO: 154 - DNA, 50 bp, Gossypium hirsutum; identical to SEQ ID NO: 141
  source 1..50: mol_type = genomic DNA

SEQ ID NO: 155 - DNA, 50 bp, Gossypium hirsutum; identical to SEQ ID NO: 143
  source 1..50: mol_type = genomic DNA

SEQ ID NO: 156 - DNA, 50 bp, Gossypium hirsutum; identical to SEQ ID NO: 145
  source 1..50: mol_type = genomic DNA

SEQ ID NO: 157 - DNA, 50 bp, Gossypium hirsutum; identical to SEQ ID NO: 147
  source 1..50: mol_type = genomic DNA

SEQ ID NO: 158 - DNA, 50 bp, Gossypium hirsutum
  source 1..50: mol_type = genomic DNA
aagcgacatgtaactgtaactccccatacctagcatagacgacaaggcca

SEQ ID NO: 159 - DNA, 50 bp, Gossypium hirsutum
  source 1..50: mol_type = genomic DNA
aaaacacacgataaaatctaaataaagacaaatccttatgccgagatgat

SEQ ID NO: 160 - DNA, 50 bp, Gossypium hirsutum
  source 1..50: mol_type = genomic DNA
aacgataaaagcatataactgagcttcccacccaccgacccgatcaagtc

SEQ ID NO: 161 - DNA, 50 bp, Gossypium hirsutum
  source 1..50: mol_type = genomic DNA
aggacccaactatttacgtattcaatatattatcggtgtaaccaaatatc

SEQ ID NO: 162 - DNA, 50 bp, Gossypium hirsutum
  source 1..50: mol_type = genomic DNA
agagttatacagataaatatcagatcaagctataaccaaatgtagaataa

SEQ ID NO: 163 - DNA, 50 bp, Gossypium hirsutum
  source 1..50: mol_type = genomic DNA
caacccaacacaccaattacggtattcattacccacccaaccctacacac

SEQ ID NO: 164 - DNA, 50 bp, Gossypium hirsutum
  source 1..50: mol_type = genomic DNA
ttagtacactaataccagggattaagcactggttcgacacagaacgcttc

SEQ ID NO: 165 - DNA, 50 bp, Gossypium hirsutum
  source 1..50: mol_type = genomic DNA
attacagagttctatgcatacccatgtacagtagatcaattataatgata

SEQ ID NO: 166 - DNA, 50 bp, Gossypium hirsutum
  source 1..50: mol_type = genomic DNA
gcaaaatatcaatttaagtagttacattcaatttaacgacatcacaaaaa

SEQ ID NO: 167 - DNA, 50 bp, Gossypium hirsutum
  source 1..50: mol_type = genomic DNA
taacctacacgcccatgcctttggcctatgtgtacctaagttataatcag

SEQ ID NO: 168 - DNA, 50 bp, Gossypium hirsutum
  source 1..50: mol_type = genomic DNA
tgcactaacatggtcacacacagtctagacacacactcgtgtctatttcc

SEQ ID NO: 169 - DNA, 50 bp, Gossypium hirsutum
  source 1..50: mol_type = genomic DNA
cgtgtggcgtcgacaatcattttaaaatattcttttaaaaaatactttaa

SEQ ID NO: 170 - DNA, 50 bp, Gossypium hirsutum
  source 1..50: mol_type = genomic DNA
ttgatactcgaacacaagaccaaagagtagacagattcttaaccagtgaa

SEQ ID NO: 171 - DNA, 50 bp, Gossypium hirsutum
  source 1..50: mol_type = genomic DNA
acaagtcatggatagggttagtttaaaataaagtagcaaaaactttctta

SEQ ID NO: 172 - DNA, 50 bp, Gossypium hirsutum
  source 1..50: mol_type = genomic DNA
acagatatatatacactcaaatactacaaaattccacaatcaaacattca

SEQ ID NO: 173 - DNA, 50 bp, Gossypium hirsutum
  source 1..50: mol_type = genomic DNA
aaatttacttgactcaaacaaatacgaatattgctgatgaatcgaatctt

SEQ ID NO: 174 - DNA, 50 bp, Gossypium hirsutum
  source 1..50: mol_type = genomic DNA
atgaataattttcttcctcaaacttaacgtctcgatctaaaatctgaaca

SEQ ID NO: 175 - DNA, 50 bp, Gossypium hirsutum
  source 1..50: mol_type = genomic DNA
aacaatgtatgacggatcagatcgatattgtcacaacgtcgaaacgtgaa

SEQ ID NO: 176 - DNA, 50 bp, Gossypium hirsutum
  source 1..50: mol_type = genomic DNA
actgagtttacacgctttaaaatacggtaagggccgataaatctcgagct

SEQ ID NO: 177 - DNA, 50 bp, Gossypium hirsutum
  source 1..50: mol_type = genomic DNA
ttaggaacacttgatcgccaacagaaaactcgatatcttttctctttaga

SEQ ID NO: 178 - DNA, 50 bp, Gossypium hirsutum
  source 1..50: mol_type = genomic DNA
caacttaatagtattctcaatctcaaaaactagctcgagacccaaagcac

SEQ ID NO: 179 - DNA, 50 bp, Gossypium hirsutum
  source 1..50: mol_type = genomic DNA
tataaagcctcgtagggagccatctgaatactggattggaaactgttatt

SEQ ID NO: 180 - DNA, 50 bp, Gossypium hirsutum
  source 1..50: mol_type = genomic DNA
caatcacacaaccccgaagcatatcctttagaatctgaataaccctttta

SEQ ID NO: 181 - DNA, 50 bp, Gossypium hirsutum
  source 1..50: mol_type = genomic DNA
gcccaacgcttcatgaagtttcttccaaaattgagaagtaaaacacggat

SEQ ID NO: 182 - DNA, 50 bp, Gossypium hirsutum
  source 1..50: mol_type = genomic DNA
gaaacatacagctcggcgagcttctataacgagtaatatgtcctgacagc

SEQ ID NO: 183 - DNA, 50 bp, Gossypium hirsutum
  source 1..50: mol_type = genomic DNA
attttttagtgggtgttaagggtaacccaataacaaaatccatcgtcacc

SEQ ID NO: 184 - DNA, 50 bp, Gossypium hirsutum
  source 1..50: mol_type = genomic DNA
caactggtgttcagccttaaccttttgacacatcaaatagtgtgacacaa

SEQ ID NO: 185 - DNA, 50 bp, Gossypium hirsutum
  source 1..50: mol_type = genomic DNA
tctcgatacattttattcccaccaagatgcatagcataagggctaatatg

SEQ ID NO: 186 - DNA, 50 bp, Gossypium hirsutum
  source 1..50: mol_type = genomic DNA
ttcgatccttgaaatacaaaaccttattgccattcaaactgaaatccgac

SEQ ID NO: 187 - DNA, 50 bp, Gossypium hirsutum
  source 1..50: mol_type = genomic DNA
tagctgtttagttcgaatctgatcaacccaagatgtcttaacttaaagtt

SEQ ID NO: 188 - DNA, 50 bp, Gossypium hirsutum
  source 1..50: mol_type = genomic DNA
cttagttcagtcatcgctctatgactaagagcatcaactacgacattagc

SEQ ID NO: 189 - DNA, 50 bp, Gossypium hirsutum
  source 1..50: mol_type = genomic DNA
cccacctacacttcctaagatttaactccttttgagttaatagatacttg

SEQ ID NO: 190 - DNA, 50 bp, Gossypium hirsutum
  source 1..50: mol_type = genomic DNA
cttctaaatttttaaagtgaacacgattgcggccagctcaagatcgtgtg

SEQ ID NO: 191 - DNA, 50 bp, Gossypium hirsutum
  source 1..50: mol_type = genomic DNA
actttaccgttttgcatcaaaacacatcctagatcgatgtgcaacgcatc

SEQ ID NO: 192 - DNA, 50 bp, Gossypium hirsutum
  source 1..50: mol_type = genomic DNA
cttgcgtcaaaacagtcttgagcttttcgaagctagactactgttcctca

SEQ ID NO: 193 - DNA, 50 bp, Gossypium hirsutum
  source 1..50: mol_type = genomic DNA
aatcaggaagaatccttcaacaaaccttcagtagtaacttgctaaaccca

SEQ ID NO: 194 - DNA, 50 bp, Gossypium hirsutum
  source 1..50: mol_type = genomic DNA
gcttttattttcttgggatcgactcgaatcctctcggcactcacaacgtg

SEQ ID NO: 195 - DNA, 50 bp, Gossypium hirsutum
  source 1..50: mol_type = genomic DNA
catacaattgttttttctagagtgtctgaagaacaatacgaaggtgctta

SEQ ID NO: 196 - DNA, 50 bp, Gossypium hirsutum
  source 1..50: mol_type = genomic DNA
taccacaaattgatcgacatatagttgaaacactagattcattaaatata

SEQ ID NO: 197 - DNA, 50 bp, Gossypium hirsutum
  source 1..50: mol_type = genomic DNA
taatgtccataacgagtcctaaaggcagtcttgtacacatcagtctcttt

SEQ ID NO: 198 - DNA, 3684 bp, Synthetic construct
  source 1..3684: mol_type = other DNA
atgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 199 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 200 - protein, 1228 aa, Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 201 - protein, 1228 aa, Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 202 - protein, 1300 aa, unidentified
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 203 - DNA, 21 bp, Synthetic construct
  source 1..21: mol_type = other DNA
gaatttctactaagtgtagat

SEQ ID NO: 204 - DNA, 21 bp, Synthetic construct
  source 1..21: mol_type = other DNA
gtaatttctactgttgtagat

SEQ ID NO: 205 - DNA, 51 bp, Synthetic construct
  source 1..51: mol_type = other DNA
gtaatttctactgttgtagataagactcattatattctagaaatttttttt

SEQ ID NO: 206 - DNA, 51 bp, Synthetic construct
  source 1..51: mol_type = other DNA
gtaatttctactgttgtagatttcaacggataatcctcaatgttttttttt

SEQ ID NO: 207 - protein, 183 aa, Streptomyces viridochromogenes
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQI

SEQ ID NO: 208 - protein, 351 aa, Oryza sativa
MADESWRAPAIVQELAAAGVEEPPSRYLLREKDRSDVKLVAAELPEPLPVVDLSRLDGAEEATKLRVALQNWGFFLLTNHGVEASLMDSVMNLSREFFNQPIERKQKFSNLIDGKNFQIQGYGTDRVVTQDQILDWSDRLHLRVEPKEEQDLAFWPDHPESFRDVLNKYASGTKRIRDDIIQAMAKLLELDEDYFLDRLNEAPAFARFNYYPPCPRPDLVFGIRPHSDGTLLTILLVDKDVSGLQVQRDGKWSNVEATPHTLLINLGDTMEVMCNGIFRSPVHRVVTNAEKERISLAMLYSVNDEKDIEPAAGLLDENRPARYRKVSVEEFRAGIFGKFSRGERYIDSLRI

SEQ ID NO: 209 - protein, 340 aa, Stenotrophomonas maltophilia
MLTFVRNAWYVAALPEELSEKPLGRTILDTPLALYRQPDGVVAALLDICPHRFAPLSDGILVNGHLQCPYHGLEFDGGGQCVHNPHGNGARPASLNVRSFPVVERDALIWIWPGDPALADPGAIPDFGCRVDPAYRTVGGYGHVDCNYKLLVDNLMDLGHAQYVHRANAQTDAFDRLEREVIVGDGEIQALMKIPGGTPSVLMAKFLRGANTPVDAWNDIRWNKVSAMLNFIAVAPEGTPKEQSIHSRGTHILTPETEASCHYFFGSSRNFGIDDPEMDGVLRSWQAQALVKEDKVVVEAIERRRAYVEANGIRPAMLSCDEAAVRVSREIEKLEQLEAA

SEQ ID NO: 210 - protein, 455 aa, unidentified
  source 1..455: note = Agrobacterium sp. CP4 strain
MLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 211 - protein, 179 aa, Enterobacter cloacae [sequence omitted]

SEQ ID NO: 212 - DNA, 17129 bp, Synthetic construct
  source 1..17129: mol_type = other DNA
gattggctgttgactaaatgtcacaacagccggtgaagcctgaactgtgtcctccaccatactatcttccatttgatcattataactagagggcgttgacccaaacaccaagtcaggactcgtattaccaaacacaggctgtgacggagtggcagctgcagcggaagtgaatgaaaatacagaacttgaagaggcaccgatggatgacccaaagagtagatgcaccgctaggagcactaacagaagttgatggtccaaaggaaaagcctgattaatgaaccaaatgtggatgtcttgggagcttgccaagtggatccaaatgcaccaccagcaatgccactgctagagcttagagacttggtttctgaagaggaagaacttattccaaaacttgaacctgaactaaaaggtttagctacagatgccgaagacccaaaaatcgaactgccagaggaaaaggttgcattcccagtcaatccaaaagatgaagatgatgcagatgaggaaaactgaatgggcgtgctctgagttgaggtaccaatgccagttcctgcagcagatgcttgggtatttacagcattgaaaggatttaaaccctcagactgtgaagtagatgtggatgtagctggagcactggatccaacgatgtttcttccagtggtcctgactgcagctttagtggcaaaaatacgacttcctgtaccagatactgggaaagacgtaccaccaaaaaaactactacctgtgcttgttgcagatgtacccccaaagatgccattgcctgtgcttgtagctgctgatgtaccaccaaataaacccgtgcccgtgcttgtagttgcagatgtaccaccaaagataccattgcctgtgcttgtacttgcagatgtaccaccgaagataccattgcttgtgcttgtcactggggaaaatgtgctgctgaaaaagccacttccagagttcgtaatagtggaagatataccactggaactgctatttccagagtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatccttcgtaactataacggttgaggtggtaggatttgccgcgtgagacctattaccctgttatccctagtttaaacacctgcaggagttcgctcgtggccgtcacggccgggcccttaattaacctcagcgcgtgttctgctggcgatcgcaacaggcacagcgctgaggtacctttgtatctattttatctattaaaggtatgtttgggcttcaggttggaatggagtagaattgaatgggttggggagtaaattttccattcaacaagttcaatttcaaaatggctaataagttttgaactcaattttattttcaataaattccttaattttttgttccttgtttgtaaactattgacttattcgatatattttaaaattgaggtattttaaaaaaataatacaatattaaaattatttataaaatataacaaaatttatgtatagtttatttgaaaattttactatagtttcatttttatattattcctaaccatttccatttaaaattatttcaattatttcttttattaatataattgaaatttcatggatttattagacacatgatttgaaattttatgggtttattaagtattttctaacacaaaatcgcttccgcatcgttttcaattcattcagtaatagaagtaattttttaaaagaaccaaatttgccaaattttgagttccataaggactctgaaaactcattatgtctattactcttcactaattgtagagacttaaattcaagaaaagagacactaattgatgataattgcccaaaaaataaaaataaaaatgtttcttccccatcctcaacctccatgaattcacagagcccaaagattaattattgggccccaattcctactcatatataccttacagtccctcaaagaaatcttaggaagtaatcaatttctgtttattcaagatgtagcctcccaaaagaaaaatacatcacatcaaattcaaacaaaaatatctacagctagcaaaacctcaaaccgttaaaatttcaagccacataaatgaaattttcatctgaaaaaaggacaatctatctagacgttagatttcagccctaatatgaatctgaagcatttggtggacgagaaagagccatgtaggaatgcatcaaacaaaggaaaaatctttgaactccaatgggattgaagatacagataccaatggataagaatctgttctctttgcccactatttaaactcaccaaacccaccagtatcttcctcaccacaaaatacattccaccgttgatcacaagccttattccaccacctccaacaggccggccaccaccatgaccagcaaccgaaagaacgagaacgagatcatcaacgccctgtccataccggccgtgtcaaaccactccgcccagatgaacctctccaccgacgcgaggatcgaggactccctctgcatcgccgagggcaacaacatcgacccgttcgtgtctgcaagcacggtccagaccggcatcaacatcgcgggccgcatcctgggcgtgctcggcgtgcccttcgcgggtcaaatcgcctctttctactcattcctcgtgggcgagctgtggccgcgcggacgtgacccgtgggaaatcttcctggagcacgttgagcagctcatccggcagcaagtgaccgagaacaccagggacaccgcactggcacggctccagggccttggcaacagcttccgcgcctaccagcagtcgctggaggactggctggagaaccgagacgacgccagaacccgctcagttctgtacacacagtacatcgccctagagctggacttcctcaacgctatgccgctcttcgccatccgtaaccaggaagtaccgcttctgatggtgtacgcacaagcagcgaacctccatctgctcctgctgcgagacgcatctctgttcggcagtgagttcgggctgacgagccaggagatccagcgctactacgagcgccaagtggagaagactcgtgagtacagcgactactgcgcgcgctggtacaacacgggcttgaacaaccttcgcgggacaaacgccgaatcctggcttcgctacaaccagttccgccgcgacctcacgctgggtgtgctggacctggtcgcgctcttcccgtcctacgacacacgggtgtacccaatgaacacgagcgcacagctcacccgtgagatctacacagatcccatcggccgcaccaacgctcccagtggcttcgcaagcacgaattggttcaacaataacgctccttctttctctgccatcgaggccgctgtcatcagaccgccgcacttactcgatttcccggagcagctcactatcttctctgtgttgtcccggtggtcgaacacgcagtacatgaactactgggtgggccacaggctagagagccggaccatccgtggcagtctctcaacctcgacccacggcaacacgaacacgagcatcaaccctgtcactctccagtttacatctagggacgtttacaggacagagtcgttcgctggcattaacattctgttgaccactccggtgaacggcgtcccttgggcccgcttcaactggaggaatcctctgaactcactgcgcggcagccttctctacactatcggctacaccggcgttgggacgcaactcttcgactcggagaccgagctgccgcccgagaccaccgagcggcctaactacgagagttattcacacaggctctccaacatccgcttgatttctgggaacaccttgcgggctccggtgtactcctggacgcaccgcagcgccgacagaactaataccatcagctccgactcgatcacccagatcccgctggtgaaggctcacacgcttcagtcgggcaccacagtcgtcaagggccctggcttcaccggcggcgacatcctgcgtcgcacatctggcggacccttcgccttcagcaacgtgaacttggacttcaatttgtcacagcggtatcgtgccagaatccggtacgccagcactacgaacctgcgaatctatgttactgtggcgggcgagcggatcttcgccgggcaattcgacaagacgatggacgcgggagcacctctgacattccagtcattctcttacgccacgatcaacacggcattcacgtttccggagcgttccagtagcctgaccgtgggcgctgataccttcagtagcgggaacgaggtgtacgttgaccgtttcgagctgatcccggtcaccgccaccacggcgaccttcgaggcggagtatgacttggagcgggctcaggaggccgtcaacgcgctgttcacaaacaccaatcctcgccgcctcaagacgggtgtgactgattaccacattgacgaggtctccaacttggtcgcgtgtctgtccgatgagttctgcctggacgagaagcgggaactgctggagaaggtcaagtacgccaagcgcctctccgacgaaaggaacctcctccaagatcccaactttacttccattaacaagcagccggacttcatctccaccaacgagcagtccaacttcacctcaatccacgagcagtcggagcacgggtggtggggcagcgagaacatcaccatccaagagggcaacgacgtcttcaaggagaactacgtgatcctgcccggcaccttcaacgagtgttacccgacctatctctaccagaagattggcgaagcggaactcaaggcttacacccgttaccaactgagtggctacattgaggactcacaagacctggaaatctacctgatccgctacaacgccaagcacgagaccctcgacgtgcctggcacggagtccgtctggcccttgagcgtggagtctcctatcggtcgttgcggcgagcccaatcgctgcgctccgcactttgagtggaatcctgatttggattgctcctgccgagacggtgagaaatgcgcccaccactcgcaccacttcagcctagacatcgacgtgggctgcatcgacctgcacgagaacttgggcgtctgggtcgtgttcaagatcaagacacaggagggccatgctcggcttgggaacctggagttcatcgaggagaagccactgctgggtgaagccttgtcacgggtgaaacgcgccgagaagaagtggcgggacaaacgggagaagctccagttggagacaaagcgtgtgtacacagaggccaaggaggccgtggatgccttgttcgtggacagtcagtacgacaggctgcaagcggacaccaacatcgggatgatccacgcggctgataagcttgttcacagaatccgcgaggcgtacctgtcagagcttagcgtgatcccaggcgtcaacgccgaaatcttcgaggaactggagggccgcattatcacggcaatctcactttatgacgcgaggaatgtggtcaagaacggtgacttcaacaacggcttggcgtgttggaacgttaaagggcacgtggatgtacaacagtcacaccacagaagtgtcttggtcatcccggagtgggaggcggaagtgagccaggccgtccgggtctgccctgggcgcggttacatcctccgcgtgacagcgtacaaggagggctacggtgagggctgcgtgacgatccacgagattgagaacaacacggacgagcttaagttcaagaactgcgaggaggaggaagtgtacccgacagacaccggcacctgcaacgactacaccgcccaccaagggaccgccgcctgcaacagccgcaacgcgggctatgaagatgcgtacgaggttgataccaccgcctcagtgaactacaaaccgacttatgaggaggagacatacacggacgtcaggcgcgacaaccattgtgagtacgaccgtggctacgtgaactatccgccggtgccagcgggctacatgacgaaggagctagaatacttccctgagacggacaaggtgtggattgaaatcggcgagaccgagggcaagtttatcgtggattctgtcgagctgctgctaatggaggagtagtagggacccgcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttgcgccggcgagtacaatcatacaagagcaatatatatatttttggttattgaaatttaaatatcatcttcacaaaatgaaaaagcacaaaaagtattaattaatatcatgttttgagactcctttttaccaagaatataaatttcacacctaagaaaattctgaactaggaaaataaccagcatacaattaaggaataagaaaatgcaattacgataaacacttgtcacaaattgtttaataagtcactatccaatcaattatcaaaagtaagatattgccacgtggcaaccagtattttcatcaccttatcaaaagataagcaaaagaaccacatcaaagccacaaaatgccaaccacagatggataaggaaaatccaaccaaccacatgtaatcccacacctcatcaccttatccacacctctgtctatatatataaacacacacttcgtaaccactcatcactcaccacaaacagagaatatctcatctcttcttagcaaacaaagcggaccgcaggtttattcttctctctctatcctctctcttctgatctcgatttgtttttttcgaatcgctctacttccagttagattcttgatttgagattaattagattgattattctaatcgttttttttgtaagcaattaagatttatcttgttttatgtttttctttaggtatgacttgttatgtatgtcactgtttcagatctgatcctctctgttggtttgtgaattctcttgtattgttctaatcactgtttctgaatttgattcgggtttttattgaattctttttatgtgttttggtatttgcaggtgggacccaccaccatggctgagatcaacaaccagaaccagtgcgtcccgtacaactgcctgagcaaccctaaggagatcatcctgggtgaggaacgcctggagaccggcaacaccgtagccgacattagcctgggcctcatcaacttcctctacagcaacttcgtgcccggcggtggcttcatcgtgggcctcctggagcttatctggggcttcatcggcccgtcccagtgggacatcttcctcgcccagatcgagcaactgatcagccagcggatcgaggagttcgctaggaaccaggccatctcccgcctggagggactctccaacctctacaaggtgtacgtgcgcgcgttcagcgactgggagaaggacccgaccaacccggccctccgcgaggaaatgcgtatccagttcaacgatatgaactcggccctcatcaccgccatcccgctcttccgcgtgcagaactacgaggtggccctcctgtccgtgtacgttcaagccgccaacctccacctctccatcctccgcgacgtgagcgtgttcggcgagcgctggggctacgacaccgccaccatcaacaaccgctactccgacctcacctccctcatccacgtttacaccaaccactgcgtggacacgtacaaccagggcctccgccgcctggagggccgcttcctctccgactggatcgtgtacaaccgcttccgccgccagctcaccatctccgtcctggacatcgtcgccttctttcccaactacgacatccgcacctaccctatccagaccgccacccagctcacccgcgaggtctacctcgacctcccgttcatcaacgagaacctcagcccggccgccgtctacccgaccttctccgccgctgagtccgccatcattcgcagcccgcacctcgtggacttcctcaactccttcaccatctacaccgactccctcgcccgcagcgcctactggggcggtcacctcgtgaactccttccgcaccggcaccactaccaacctcatccgcagcccgctctacggccgcgagggcaacaccgagcgcccggtgaccatcaccgccagcccgagcgtgcccatcttccgcaccctcagctaccccaccggcctggacaacagcaaccctgtggcgggcatcgagggcgtggagttccagaacaccatctccaggagcatctaccgcaagagcggccctatcgacagcttcagcgagctgcctcctcaggacgccagcgtgagccctgccatcggctacagccacaggctgtgccacgccaccttcctggagcgcatcagcggccctcgcatcgcgggcaccgtgttctcgtggacccaccgcagcgcctctcctacgaacgaggtgtctcctagtcgcatcacccagatcccttgggtcaaggcccacaccctggctagtggcgctagtgtcatcaagggccctggcttcaccggtggtgacatcctgaccaggaactctatgggcgagctgggcactctgagggtcactttcactggccgcctgcctcagtcttactacatccgcttccgctacgctagtgtcgctaaccgctctggtactttccgctactctcagcctccgtcttacggtatctctttccctaagactatggacgctggtgagcctctgaccagtaggagcttcgctcacactactctgttcactcctatcactttctctagggctcaggaggagttcgacctgtacatccagtctggtgtgtacatcgacaggatcgagttcatccccgtgaccgccacgttcgaggccgagtacgaccttgagcgcgcccagaaggtggtgaacgccctcttcactagcactaaccagctaggcctgaagactgacgtgaccgactaccacatcgaccaagtgagcaacctagtggcctgcctctccgacgagttctgcctcgacgagaagcgcgagctgtccgagaaggtgaagcacgccaagcgcctctccgacgagcgcaacctgctccaggaccccaacttcaggggcatcaacaggcagcccgaccgcggctggcgcggctccaccgacatcaccatccagggcggtgacgacgtattcaaggagaactacgttaccctccccggcaccttcgacgagtgttaccccacctacctctaccagaagatcgacgagtccaagctgaaggcctacacccgctaccagctccgcggctacatcgaggactcccaggacctggaaatctacctcatccgctacaacgccaagcacgagatcgtgaacgtgcctggcaccggcagcctctggcctctcagcgtggagaaccagatcggcccttgcggcgagcctaaccgctgcgcccctcacctcgagtggaaccctgacctccactgctcgtgcagggacggcgagaagtgcgcccaccatagccaccacttctctctggacatcgacgtgggctgcaccgacctgaacgaggacctgggcgtgtgggttatcttcaagatcaagacccaggacggtcacgccaggctgggtaacctggagttccttgaggaaaagcctctgctgggtgaggccctggccagggtcaagagggctgagaagaaatggagggataagagggagaccctgcagctggagaccactatcgtctacaaggaggctaaggagtctgtcgatgctctgttcgtcaactctcagtacgatagactgcaagctgataccaacatcgctatgatccacgctgcggataagcgggtccaccggatccgggaggcttaccttccggagctttctgtcatcccgggtgtcaacgctgcgatcttcgaggaacttgaggaacggatcttcactgcgtttagtctttacgatgcgcggaacatcatcaagaacggggacttcaacaatggtctgctgtgctggaacgtcaagggtcatgtcgaggtcgaggaacaaaacaatcatcgtagtgtccttgtcattcctgagtgggaggcggaggtctctcaagaggtccgtgtttgcccggggcgtgggtacattcttcgtgttactgcgtacaaggaggggtacggggaggggtgcgttactattcatgagattgagaacaatactgatgagcttaagttcaacaattgtgttgaggaggaggtttacccgaacaatactgttacgtgcatcaactacacggcaacgcaagaggaatacgaggggacgtacacctcgcgtaatagagggtatgatgaggcgtacggaaacaacccgtcggttccagcagattatgcctcggtttatgaggagaagtcgtacacggatagacgacgcgagaatccatgtgagtcaaatcgaggatacggagattacacaccattaccagcaggatacgttacaaaggagttggaatacttcccggaaacagataaagtttggattgaaatcggagaaacagaaggaacattcatcgtcgactcagtagaattgttgttgatggaagaatgagggtcccaaataaaattacttatagatagatttggaactagatgactagtgttttattgtttctggtttgtgtaattctataaattaatgttatctctatgtccaagaatgttatctctagtgctctttgttgtacttggactgaaattatggggattggatttatgtgcttttcatgactcgtcaataaatgaataaataaaatgtgacttttctcctttacttgttgtgtttggcttctgatgtaataagaaatccaatgaatctgctaatattcttgtgagagttttagtgatataataataatcctaccgcttcaaacatagtttttgtttttaaaaaaaaaaaaaaaaacaagggaaagtatattaacaatctcttagatttatcccagaataattgaatttttcatctattatgcttagtactattaagtaaagctagtgagaggggcattttatggctcatcactagagcatgtacaaaagaaattgttctcgtgaatttggcgcgcccaactatttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttctctccgctttgaattgtctcgttgtcctcctcactttcatcggccgtttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttcttttctcaatttcgttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagggccggccaccaccatggctaagcagaacaacaacttctccgtgcgcgcgctcccgtcgttcatcgacgtcttcaacggcatctacgggttcgccaccggcatccaggacatcttcaacatgatcttcggcaccgacacgggcgacctcacgctggaggaggtgctcaagaaccaggaactgctctacgacatctcgggcaagctggagggcatctccggcgacctcagcgaaattatcgcgcaagggaacctcaacacggagctggcgaaagagctgctcaagatcgccaacgagcagaacaacgtgctgacggacgtgaacaacaagctcaacgcgatcaactcgatgctccacatctacctcccaaagattacgaacatgctgtccgacgtcatgaagcagaactatgccctcagcctccagatcgagtacctctcgaagcagctccaggagatttcggataagctggacgtcatcaacctgaacgtgctgataaactccacgctcacggagatcactccggcctaccagcgcatcaagtacgtgaacgagaagttcgacgagctgacactggcgactgagaagaccctccgcgccaagcaagggtccgaggacatcattgcgaacgacacgctggagaacttgacggagttgaccgagctggccaagagcgtgacgaagaacgacatggacagcttcgagttctacctccacactttccacgacgtgctgatcggcaacaacctcttcggccggagcgcgctcaagaccgcagccgagctgattacgaaggacgagatcaagacctccgggtccgagatcggaaaggtgtacagcttcctgatcgtcctcacgtgcttacaagctaaggcgttcctcaccctgaccgcctgccgtaagctgttgggcctgtccgacatcgactacaccaacatcctcaaccagcacctcaacgacgagaagaacgtctttcgggacaacatcctcccgacactgagcaacaagttcagtaacccgaactacgtcaagacgattggcagcgacaactacgccaaggttatcctagaggccgagcccggttacgccctggtcgggttcgagatcatcaacgaccgcatcccggtcctcaaggcgtacaaggccaagctcaagcagaactaccaagtggaccaccagagcctcagcgagatcgtgtacctggacatcgacaagttgttctgcccgaagaactcggagcagaagtattacaccaagagcctgaccttcccggacggctacgttattacaaagatcacgttcgagaagaagctcaacaacctccgttacgaggcaactgccaacttctacgacccgtccaccggcgacattgacttgaacgagaagcaagtcgagtccacgttcctccaggccgactacatcagcatcaacgtcagcgacgacgacggcgtgtacatgccgctcggagtcatcagcgagaccttcctcagcccgatcaactcgttcgagctggaggtggacgagaagtccaagattctcaccctgacctgcaagagctacctccgggaatacctcctggagagcgacctcatcaataaggagacttcgctcatagctccgcccaacgtcttcatctccaacatcgtcgagaactggaacatcgaggccgacaacctggagccgtgggtggcaaacaacaagaacgcctacgtggactccaccggcgggatcgagggaagcaaggccctgttcacccagggcgacggtgagttctcgcagttcatcggcgacaagctcaagcccaacacggactacatcatccagtacaccgtcaagggcaagccagcgatctacctcaagaacaagaacaccgggtacaccatgtacgaggacacgaacggcagcagcgaggagttccagaccatcgcggtcaactacacctccgagaccgatccctcccagacccaccttgtcttcaagtcccagagcggctacgaggcgtggggcgacaacttcatcatcctggagtgcaaggctttcgagactcccgagggcccggagcttatcaagttcgatgactggatttcgtttggcaccacctacatccgggacgacgtgctaaccatcgacccgtcgcgcggcggctacttccgccaaagcctcaagctggactcgtactccacgtacaacctatctttcagcttctcgggcctgtgggcgaaggtgattatcaagaactcgcacggcgtggtcctgttcgagaaggtctcccagcagtcgtcatacgtggacatcagcgagtccttcaccaccaccagcaacaaggagggcttcttcatcgagctgaccggcgactcgcgcggcgggttcggctccttccgcgacttctccatgaaggagaaattcgagtgatagggaccctttgctttatatatgataagaaattgtttttttagttttgctttccatttgtaaaattatcttttgagagtttttagattattgcactttctaaattttcattttgcttctttgttggatctgggatttctgaaatgaacattagagtaataaatcgaatctgagtattacaagcaagtttgttgaaataatgatctctaattttgtcaagttaaagaaatcaaaatatcttgtaccaaaaaaaaaaaaaaatcaaaacatcatgtttgattaacaaaactccaatttgaaatcaatttaaatttaaatttcatttttagaaagaaaaaaaaaatattattccatccattattagttctcgatttcacacaactagtttttcgatttactcacgtacctacatatgagtatgtaagggttgattacgtattcattattgctctttgtttcacataattcttttttttcaatcaaaatgaaaattatataaaggagtacaataactataacggtcctaaggtagcgaaataacttcgtatagcatacattatacgaagttatatagtactttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagacttgcagcacttgtaccaccaaaaatactacttccagagccagtaaaagcagatgtactaccaaaaggaaagatacctacattgccaagttttgtatcctgtgtcttagattcagttgcttcccctgaagttgctgataatgttgaaactgaagttgatgggtccccagaggatgaaaacttgaaaacaggtgcacctgtaaaggaagggattgaagcctccacagaggacctgctggtaaagctgatgctggcattcgcagttgcacttgcagaggtgatggtagcagcagaagtgtcatttttggtggcagttgcactggaggaactttgacaattactcgggactagagaaggcaccggagaagagaacgaaaaaggactagaagcacaagacccatgattaaaggtagaaccatctgctgacgtatcaaacttgaacttccagctgatattgaggttgatattgaagttgattggtgttgcgaagtggacataatggcaaactcaggatgcaaatctgcgagctcatttaagtttacgcgctctacatctcatccaaagttgcaaactcatccatcactgagagctcatttgaagatgtgagttcacccaaagatgcgagctcatcaaaagatgtgaactattagtataatttattcaaaaaaattcgaaactcaaatccatatattttgcatgtatgcagatgaggtttgatggtgaaacaagagctacagaaatcaatggtactgagttgcacttcaatagtgtcactggcattctccttctaagcatgccatcaatggcaggaggtaatatatataattttgatatctccatgtagtcatggttttttttttaattatataatctcgtgttataatttcattgtaatagaaccaattaaaaaatattttttaattttaaaattcaggtttgatatagaggcacacttgattcatgaaagtgcatacggtaagattgttgtaatagggatcatg

SEQ ID NO: 213 - DNA, 15129 bp, Synthetic construct
  source 1..15129: mol_type = other DNA
agtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatccttcgtaactataacggttgaggtggtaggatttgccgcgtgagacctattaccctgttatccctagtttaaacacctgcaggagttcgctcgtggccgtcacggccgggcccttaattaacctcagcgcgtgttctgctggcgatcgcaacaggcacagcgctgaggtacctttgtatctattttatctattaaaggtatgtttgggcttcaggttggaatggagtagaattgaatgggttggggagtaaattttccattcaacaagttcaatttcaaaatggctaataagttttgaactcaattttattttcaataaattccttaattttttgttccttgtttgtaaactattgacttattcgatatattttaaaattgaggtattttaaaaaaataatacaatattaaaattatttataaaatataacaaaatttatgtatagtttatttgaaaattttactatagtttcatttttatattattcctaaccatttccatttaaaattatttcaattatttcttttattaatataattgaaatttcatggatttattagacacatgatttgaaattttatgggtttattaagtattttctaacacaaaatcgcttccgcatcgttttcaattcattcagtaatagaagtaattttttaaaagaaccaaatttgccaaattttgagttccataaggactctgaaaactcattatgtctattactcttcactaattgtagagacttaaattcaagaaaagagacactaattgatgataattgcccaaaaaataaaaataaaaatgtttcttccccatcctcaacctccatgaattcacagagcccaaagattaattattgggccccaattcctactcatatataccttacagtccctcaaagaaatcttaggaagtaatcaatttctgtttattcaagatgtagcctcccaaaagaaaaatacatcacatcaaattcaaacaaaaatatctacagctagcaaaacctcaaaccgttaaaatttcaagccacataaatgaaattttcatctgaaaaaaggacaatctatctagacgttagatttcagccctaatatgaatctgaagcatttggtggacgagaaagagccatgtaggaatgcatcaaacaaaggaaaaatctttgaactccaatgggattgaagatacagataccaatggataagaatctgttctctttgcccactatttaaactcaccaaacccaccagtatcttcctcaccacaaaatacattccaccgttgatcacaagccttattccaccacctccaacaggccggccaccaccatgaccagcaaccgaaagaacgagaacgagatcatcaacgccctgtccataccggccgtgtcaaaccactccgcccagatgaacctctccaccgacgcgaggatcgaggactccctctgcatcgccgagggcaacaacatcgacccgttcgtgtctgcaagcacggtccagaccggcatcaacatcgcgggccgcatcctgggcgtgctcggcgtgcccttcgcgggtcaaatcgcctctttctactcattcctcgtgggcgagctgtggccgcgcggacgtgacccgtgggaaatcttcctggagcacgttgagcagctcatccggcagcaagtgaccgagaacaccagggacaccgcactggcacggctccagggccttggcaacagcttccgcgcctaccagcagtcgctggaggactggctggagaaccgagacgacgccagaacccgctcagttctgtacacacagtacatcgccctagagctggacttcctcaacgctatgccgctcttcgccatccgtaaccaggaagtaccgcttctgatggtgtacgcacaagcagcgaacctccatctgctcctgctgcgagacgcatctctgttcggcagtgagttcgggctgacgagccaggagatccagcgctactacgagcgccaagtggagaagactcgtgagtacagcgactactgcgcgcgctggtacaacacgggcttgaacaaccttcgcgggacaaacgccgaatcctggcttcgctacaaccagttccgccgcgacctcacgctgggtgtgctggacctggtcgcgctcttcccgtcctacgacacacgggtgtacccaatgaacacgagcgcacagctcacccgtgagatctacacagatcccatcggccgcaccaacgctcccagtggcttcgcaagcacgaattggttcaacaataacgctccttctttctctgccatcgaggccgctgtcatcagaccgccgcacttactcgatttcccggagcagctcactatcttctctgtgttgtcccggtggtcgaacacgcagtacatgaactactgggtgggccacaggctagagagccggaccatccgtggcagtctctcaacctcgacccacggcaacacgaacacgagcatcaaccctgtcactctccagtttacatctagggacgtttacaggacagagtcgttcgctggcattaacattctgttgaccactccggtgaacggcgtcccttgggcccgcttcaactggaggaatcctctgaactcactgcgcggcagccttctctacactatcggctacaccggcgttgggacgcaactcttcgactcggagaccgagctgccgcccgagaccaccgagcggcctaactacgagagttattcacacaggctctccaacatccgcttgatttctgggaacaccttgcgggctccggtgtactcctggacgcaccgcagcgccgacagaactaataccatcagctccgactcgatcacccagatcccgctggtgaaggctcacacgcttcagtcgggcaccacagtcgtcaagggccctggcttcaccggcggcgacatcctgcgtcgcacatctggcggacccttcgccttcagcaacgtgaacttggacttcaatttgtcacagcggtatcgtgccagaatccggtacgccagcactacgaacctgcgaatctatgttactgtggcgggcgagcggatcttcgccgggcaattcgacaagacgatggacgcgggagcacctctgacattccagtcattctcttacgccacgatcaacacggcattcacgtttccggagcgttccagtagcctgaccgtgggcgctgataccttcagtagcgggaacgaggtgtacgttgaccgtttcgagctgatcccggtcaccgccaccacggcgaccttcgaggcggagtatgacttggagcgggctcaggaggccgtcaacgcgctgttcacaaacaccaatcctcgccgcctcaagacgggtgtgactgattaccacattgacgaggtctccaacttggtcgcgtgtctgtccgatgagttctgcctggacgagaagcgggaactgctggagaaggtcaagtacgccaagcgcctctccgacgaaaggaacctcctccaagatcccaactttacttccattaacaagcagccggacttcatctccaccaacgagcagtccaacttcacctcaatccacgagcagtcggagcacgggtggtggggcagcgagaacatcaccatccaagagggcaacgacgtcttcaaggagaactacgtgatcctgcccggcaccttcaacgagtgttacccgacctatctctaccagaagattggcgaagcggaactcaaggcttacacccgttaccaactgagtggctacattgaggactcacaagacctggaaatctacctgatccgctacaacgccaagcacgagaccctcgacgtgcctggcacggagtccgtctggcccttgagcgtggagtctcctatcggtcgttgcggcgagcccaatcgctgcgctccgcactttgagtggaatcctgatttggattgctcctgccgagacggtgagaaatgcgcccaccactcgcaccacttcagcctagacatcgacgtgggctgcatcgacctgcacgagaacttgggcgtctgggtcgtgttcaagatcaagacacaggagggccatgctcggcttgggaacctggagttcatcgaggagaagccactgctgggtgaagccttgtcacgggtgaaacgcgccgagaagaagtggcgggacaaacgggagaagctccagttggagacaaagcgtgtgtacacagaggccaaggaggccgtggatgccttgttcgtggacagtcagtacgacaggctgcaagcggacaccaacatcgggatgatccacgcggctgataagcttgttcacagaatccgcgaggcgtacctgtcagagcttagcgtgatcccaggcgtcaacgccgaaatcttcgaggaactggagggccgcattatcacggcaatctcactttatgacgcgaggaatgtggtcaagaacggtgacttcaacaacggcttggcgtgttggaacgttaaagggcacgtggatgtacaacagtcacaccacagaagtgtcttggtcatcccggagtgggaggcggaagtgagccaggccgtccgggtctgccctgggcgcggttacatcctccgcgtgacagcgtacaaggagggctacggtgagggctgcgtgacgatccacgagattgagaacaacacggacgagcttaagttcaagaactgcgaggaggaggaagtgtacccgacagacaccggcacctgcaacgactacaccgcccaccaagggaccgccgcctgcaacagccgcaacgcgggctatgaagatgcgtacgaggttgataccaccgcctcagtgaactacaaaccgacttatgaggaggagacatacacggacgtcaggcgcgacaaccattgtgagtacgaccgtggctacgtgaactatccgccggtgccagcgggctacatgacgaaggagctagaatacttccctgagacggacaaggtgtggattgaaatcggcgagaccgagggcaagtttatcgtggattctgtcgagctgctgctaatggaggagtagtagggacccgcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttgcgccggcgagtacaatcatacaagagcaatatatatatttttggttattgaaatttaaatatcatcttcacaaaatgaaaaagcacaaaaagtattaattaatatcatgttttgagactcctttttaccaagaatataaatttcacacctaagaaaattctgaactaggaaaataaccagcatacaattaaggaataagaaaatgcaattacgataaacacttgtcacaaattgtttaataagtcactatccaatcaattatcaaaagtaagatattgccacgtggcaaccagtattttcatcaccttatcaaaagataagcaaaagaaccacatcaaagccacaaaatgccaaccacagatggataaggaaaatccaaccaaccacatgtaatcccacacctcatcaccttatccacacctctgtctatatatataaacacacacttcgtaaccactcatcactcaccacaaacagagaatatctcatctcttcttagcaaacaaagcggaccgcaggtttattcttctctctctatcctctctcttctgatctcgatttgtttttttcgaatcgctctacttccagttagattcttgatttgagattaattagattgattattctaatcgttttttttgtaagcaattaagatttatcttgttttatgtttttctttaggtatgacttgttatgtatgtcactgtttcagatctgatcctctctgttggtttgtgaattctcttgtattgttctaatcactgtttctgaatttgattcgggtttttattgaattctttttatgtgttttggtatttgcaggtgggacccaccaccatggctgagatcaacaaccagaaccagtgcgtcccgtacaactgcctgagcaaccctaaggagatcatcctgggtgaggaacgcctggagaccggcaacaccgtagccgacattagcctgggcctcatcaacttcctctacagcaacttcgtgcccggcggtggcttcatcgtgggcctcctggagcttatctggggcttcatcggcccgtcccagtgggacatcttcctcgcccagatcgagcaactgatcagccagcggatcgaggagttcgctaggaaccaggccatctcccgcctggagggactctccaacctctacaaggtgtacgtgcgcgcgttcagcgactgggagaaggacccgaccaacccggccctccgcgaggaaatgcgtatccagttcaacgatatgaactcggccctcatcaccgccatcccgctcttccgcgtgcagaactacgaggtggccctcctgtccgtgtacgttcaagccgccaacctccacctctccatcctccgcgacgtgagcgtgttcggcgagcgctggggctacgacaccgccaccatcaacaaccgctactccgacctcacctccctcatccacgtttacaccaaccactgcgtggacacgtacaaccagggcctccgccgcctggagggccgcttcctctccgactggatcgtgtacaaccgcttccgccgccagctcaccatctccgtcctggacatcgtcgccttctttcccaactacgacatccgcacctaccctatccagaccgccacccagctcacccgcgaggtctacctcgacctcccgttcatcaacgagaacctcagcccggccgccgtctacccgaccttctccgccgctgagtccgccatcattcgcagcccgcacctcgtggacttcctcaactccttcaccatctacaccgactccctcgcccgcagcgcctactggggcggtcacctcgtgaactccttccgcaccggcaccactaccaacctcatccgcagcccgctctacggccgcgagggcaacaccgagcgcccggtgaccatcaccgccagcccgagcgtgcccatcttccgcaccctcagctaccccaccggcctggacaacagcaaccctgtggcgggcatcgagggcgtggagttccagaacaccatctccaggagcatctaccgcaagagcggccctatcgacagcttcagcgagctgcctcctcaggacgccagcgtgagccctgccatcggctacagccacaggctgtgccacgccaccttcctggagcgcatcagcggccctcgcatcgcgggcaccgtgttctcgtggacccaccgcagcgcctctcctacgaacgaggtgtctcctagtcgcatcacccagatcccttgggtcaaggcccacaccctggctagtggcgctagtgtcatcaagggccctggcttcaccggtggtgacatcctgaccaggaactctatgggcgagctgggcactctgagggtcactttcactggccgcctgcctcagtcttactacatccgcttccgctacgctagtgtcgctaaccgctctggtactttccgctactctcagcctccgtcttacggtatctctttccctaagactatggacgctggtgagcctctgaccagtaggagcttcgctcacactactctgttcactcctatcactttctctagggctcaggaggagttcgacctgtacatccagtctggtgtgtacatcgacaggatcgagttcatccccgtgaccgccacgttcgaggccgagtacgaccttgagcgcgcccagaaggtggtgaacgccctcttcactagcactaaccagctaggcctgaagactgacgtgaccgactaccacatcgaccaagtgagcaacctagtggcctgcctctccgacgagttctgcctcgacgagaagcgcgagctgtccgagaaggtgaagcacgccaagcgcctctccgacgagcgcaacctgctccaggaccccaacttcaggggcatcaacaggcagcccgaccgcggctggcgcggctccaccgacatcaccatccagggcggtgacgacgtattcaaggagaactacgttaccctccccggcaccttcgacgagtgttaccccacctacctctaccagaagatcgacgagtccaagctgaaggcctacacccgctaccagctccgcggctacatcgaggactcccaggacctggaaatctacctcatccgctacaacgccaagcacgagatcgtgaacgtgcctggcaccggcagcctctggcctctcagcgtggagaaccagatcggcccttgcggcgagcctaaccgctgcgcccctcacctcgagtggaaccctgacctccactgctcgtgcagggacggcgagaagtgcgcccaccatagccaccacttctctctggacatcgacgtgggctgcaccgacctgaacgaggacctgggcgtgtgggttatcttcaagatcaagacccaggacggtcacgccaggctgggtaacctggagttccttgaggaaaagcctctgctgggtgaggccctggccagggtcaagagggctgagaagaaatggagggataagagggagaccctgcagctggagaccactatcgtctacaaggaggctaaggagtctgtcgatgctctgttcgtcaactctcagtacgatagactgcaagctgataccaacatcgctatgatccacgctgcggataagcgggtccaccggatccgggaggcttaccttccggagctttctgtcatcccgggtgtcaacgctgcgatcttcgaggaacttgaggaacggatcttcactgcgtttagtctttacgatgcgcggaacatcatcaagaacggggacttcaacaatggtctgctgtgctggaacgtcaagggtcatgtcgaggtcgaggaacaaaacaatcatcgtagtgtccttgtcattcctgagtgggaggcggaggtctctcaagaggtccgtgtttgcccggggcgtgggtacattcttcgtgttactgcgtacaaggaggggtacggggaggggtgcgttactattcatgagattgagaacaatactgatgagcttaagttcaacaattgtgttgaggaggaggtttacccgaacaatactgttacgtgcatcaactacacggcaacgcaagaggaatacgaggggacgtacacctcgcgtaatagagggtatgatgaggcgtacggaaacaacccgtcggttccagcagattatgcctcggtttatgaggagaagtcgtacacggatagacgacgcgagaatccatgtgagtcaaatcgaggatacggagattacacaccattaccagcaggatacgttacaaaggagttggaatacttcccggaaacagataaagtttggattgaaatcggagaaacagaaggaacattcatcgtcgactcagtagaattgttgttgatggaagaatgagggtcccaaataaaattacttatagatagatttggaactagatgactagtgttttattgtttctggtttgtgtaattctataaattaatgttatctctatgtccaagaatgttatctctagtgctctttgttgtacttggactgaaattatggggattggatttatgtgcttttcatgactcgtcaataaatgaataaataaaatgtgacttttctcctttacttgttgtgtttggcttctgatgtaataagaaatccaatgaatctgctaatattcttgtgagagttttagtgatataataataatcctaccgcttcaaacatagtttttgtttttaaaaaaaaaaaaaaaaacaagggaaagtatattaacaatctcttagatttatcccagaataattgaatttttcatctattatgcttagtactattaagtaaagctagtgagaggggcattttatggctcatcactagagcatgtacaaaagaaattgttctcgtgaatttggcgcgcccaactatttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttctctccgctttgaattgtctcgttgtcctcctcactttcatcggccgtttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttcttttctcaatttcgttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagggccggccaccaccatggctaagcagaacaacaacttctccgtgcgcgcgctcccgtcgttcatcgacgtcttcaacggcatctacgggttcgccaccggcatccaggacatcttcaacatgatcttcggcaccgacacgggcgacctcacgctggaggaggtgctcaagaaccaggaactgctctacgacatctcgggcaagctggagggcatctccggcgacctcagcgaaattatcgcgcaagggaacctcaacacggagctggcgaaagagctgctcaagatcgccaacgagcagaacaacgtgctgacggacgtgaacaacaagctcaacgcgatcaactcgatgctccacatctacctcccaaagattacgaacatgctgtccgacgtcatgaagcagaactatgccctcagcctccagatcgagtacctctcgaagcagctccaggagatttcggataagctggacgtcatcaacctgaacgtgctgataaactccacgctcacggagatcactccggcctaccagcgcatcaagtacgtgaacgagaagttcgacgagctgacactggcgactgagaagaccctccgcgccaagcaagggtccgaggacatcattgcgaacgacacgctggagaacttgacggagttgaccgagctggccaagagcgtgacgaagaacgacatggacagcttcgagttctacctccacactttccacgacgtgctgatcggcaacaacctcttcggccggagcgcgctcaagaccgcagccgagctgattacgaaggacgagatcaagacctccgggtccgagatcggaaaggtgtacagcttcctgatcgtcctcacgtgcttacaagctaaggcgttcctcaccctgaccgcctgccgtaagctgttgggcctgtccgacatcgactacaccaacatcctcaaccagcacctcaacgacgagaagaacgtctttcgggacaacatcctcccgacactgagcaacaagttcagtaacccgaactacgtcaagacgattggcagcgacaactacgccaaggttatcctagaggccgagcccggttacgccctggtcgggttcgagatcatcaacgaccgcatcccggtcctcaaggcgtacaaggccaagctcaagcagaactaccaagtggaccaccagagcctcagcgagatcgtgtacctggacatcgacaagttgttctgcccgaagaactcggagcagaagtattacaccaagagcctgaccttcccggacggctacgttattacaaagatcacgttcgagaagaagctcaacaacctccgttacgaggcaactgccaacttctacgacccgtccaccggcgacattgacttgaacgagaagcaagtcgagtccacgttcctccaggccgactacatcagcatcaacgtcagcgacgacgacggcgtgtacatgccgctcggagtcatcagcgagaccttcctcagcccgatcaactcgttcgagctggaggtggacgagaagtccaagattctcaccctgacctgcaagagctacctccgggaatacctcctggagagcgacctcatcaataaggagacttcgctcatagctccgcccaacgtcttcatctccaacatcgtcgagaactggaacatcgaggccgacaacctggagccgtgggtggcaaacaacaagaacgcctacgtggactccaccggcgggatcgagggaagcaaggccctgttcacccagggcgacggtgagttctcgcagttcatcggcgacaagctcaagcccaacacggactacatcatccagtacaccgtcaagggcaagccagcgatctacctcaagaacaagaacaccgggtacaccatgtacgaggacacgaacggcagcagcgaggagttccagaccatcgcggtcaactacacctccgagaccgatccctcccagacccaccttgtcttcaagtcccagagcggctacgaggcgtggggcgacaacttcatcatcctggagtgcaaggctttcgagactcccgagggcccggagcttatcaagttcgatgactggatttcgtttggcaccacctacatccgggacgacgtgctaaccatcgacccgtcgcgcggcggctacttccgccaaagcctcaagctggactcgtactccacgtacaacctatctttcagcttctcgggcctgtgggcgaaggtgattatcaagaactcgcacggcgtggtcctgttcgagaaggtctcccagcagtcgtcatacgtggacatcagcgagtccttcaccaccaccagcaacaaggagggcttcttcatcgagctgaccggcgactcgcgcggcgggttcggctccttccgcgacttctccatgaaggagaaattcgagtgatagggaccctttgctttatatatgataagaaattgtttttttagttttgctttccatttgtaaaattatcttttgagagtttttagattattgcactttctaaattttcattttgcttctttgttggatctgggatttctgaaatgaacattagagtaataaatcgaatctgagtattacaagcaagtttgttgaaataatgatctctaattttgtcaagttaaagaaatcaaaatatcttgtaccaaaaaaaaaaaaaaatcaaaacatcatgtttgattaacaaaactccaatttgaaatcaatttaaatttaaatttcatttttagaaagaaaaaaaaaatattattccatccattattagttctcgatttcacacaactagtttttcgatttactcacgtacctacatatgagtatgtaagggttgattacgtattcattattgctctttgtttcacataattcttttttttcaatcaaaatgaaaattatataaaggagtacaataactataacggtcctaaggtagcgaaataacttcgtatagcatacattatacgaagttatatagtactttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaaga

SEQ ID NO: 214 - DNA, 50 bp, Synthetic construct
  source 1..50: mol_type = other DNA
ataccactggaactgctatttccagagtcagcatcatcacaccaaaagtt

SEQ ID NO: 215 - DNA, 100 bp, Synthetic construct
  source 1..100: mol_type = other DNA
tccagagttcgtaatagtggaagatataccactggaactgctatttccagagtcagcatcatcacaccaaaagttaggcccgaatagtttgaaattagaa

SEQ ID NO: 216 - DNA, 200 bp, Synthetic construct
  source 1..200: mol_type = other DNA
ccattgcttgtgcttgtcactggggaaaatgtgctgctgaaaaagccacttccagagttcgtaatagtggaagatataccactggaactgctatttccagagtcagcatcatcacaccaaaagttaggcccgaatagtttgaaattagaaagctcgcaattgaggtctacaggccaaattcgctcttagccgtacaatat

SEQ ID NO: 217 - DNA, 1200 bp, Synthetic construct
  source 1..1200: mol_type = other DNA
gattggctgttgactaaatgtcacaacagccggtgaagcctgaactgtgtcctccaccatactatcttccatttgatcattataactagagggcgttgacccaaacaccaagtcaggactcgtattaccaaacacaggctgtgacggagtggcagctgcagcggaagtgaatgaaaatacagaacttgaagaggcaccgatggatgacccaaagagtagatgcaccgctaggagcactaacagaagttgatggtccaaaggaaaagcctgattaatgaaccaaatgtggatgtcttgggagcttgccaagtggatccaaatgcaccaccagcaatgccactgctagagcttagagacttggtttctgaagaggaagaacttattccaaaacttgaacctgaactaaaaggtttagctacagatgccgaagacccaaaaatcgaactgccagaggaaaaggttgcattcccagtcaatccaaaagatgaagatgatgcagatgaggaaaactgaatgggcgtgctctgagttgaggtaccaatgccagttcctgcagcagatgcttgggtatttacagcattgaaaggatttaaaccctcagactgtgaagtagatgtggatgtagctggagcactggatccaacgatgtttcttccagtggtcctgactgcagctttagtggcaaaaatacgacttcctgtaccagatactgggaaagacgtaccaccaaaaaaactactacctgtgcttgttgcagatgtacccccaaagatgccattgcctgtgcttgtagctgctgatgtaccaccaaataaacccgtgcccgtgcttgtagttgcagatgtaccaccaaagataccattgcctgtgcttgtacttgcagatgtaccaccgaagataccattgcttgtgcttgtcactggggaaaatgtgctgctgaaaaagccacttccagagttcgtaatagtggaagatataccactggaactgctatttccagagtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtc

SEQ ID NO: 218 - DNA, 50 bp, Synthetic construct
  source 1..50: mol_type = other DNA
catgtagatttcccggacatgaagacttgcagcacttgtaccaccaaaaa

SEQ ID NO: 219 - DNA, 100 bp, Synthetic construct
  source 1..100: mol_type = other DNA
ttgaccatcatactcattgctgatccatgtagatttcccggacatgaagacttgcagcacttgtaccaccaaaaatactacttccagagccagtaaaagc

SEQ ID NO: 220 - DNA, 200 bp, Synthetic construct
  source 1..200: mol_type = other DNA
catttttgaattgaaaaaaaattggtaattactctttctttttctccatattgaccatcatactcattgctgatccatgtagatttcccggacatgaagacttgcagcacttgtaccaccaaaaatactacttccagagccagtaaaagcagatgtactaccaaaaggaaagatacctacattgccaagttttgtatcct

SEQ ID NO: 221 - DNA, 1200 bp, Synthetic construct
  source 1..1200: mol_type = other DNA
gaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagacttgcagcacttgtaccaccaaaaatactacttccagagccagtaaaagcagatgtactaccaaaaggaaagatacctacattgccaagttttgtatcctgtgtcttagattcagttgcttcccctgaagttgctgataatgttgaaactgaagttgatgggtccccagaggatgaaaacttgaaaacaggtgcacctgtaaaggaagggattgaagcctccacagaggacctgctggtaaagctgatgctggcattcgcagttgcacttgcagaggtgatggtagcagcagaagtgtcatttttggtggcagttgcactggaggaactttgacaattactcgggactagagaaggcaccggagaagagaacgaaaaaggactagaagcacaagacccatgattaaaggtagaaccatctgctgacgtatcaaacttgaacttccagctgatattgaggttgatattgaagttgattggtgttgcgaagtggacataatggcaaactcaggatgcaaatctgcgagctcatttaagtttacgcgctctacatctcatccaaagttgcaaactcatccatcactgagagctcatttgaagatgtgagttcacccaaagatgcgagctcatcaaaagatgtgaactattagtataatttattcaaaaaaattcgaaactcaaatccatatattttgcatgtatgcagatgaggtttgatggtgaaacaagagctacagaaatcaatggtactgagttgcacttcaatagtgtcactggcattctccttctaagcatgccatcaatggcaggaggtaatatatataattttgatatctccatgtagtcatggttttttttttaattatataatctcgtgttataatttcattgtaatagaaccaattaaaaaatattttttaattttaaaattcaggtttgatatagaggcacacttgattcatgaaagtgcatacggtaagattgttgtaatagggatcatg

SEQ ID NO: 222 - DNA, 1000 bp, Gossypium hirsutum
  source 1..1000: mol_type = genomic DNA
gattggctgttgactaaatgtcacaacagccggtgaagcctgaactgtgtcctccaccatactatcttccatttgatcattataactagagggcgttgacccaaacaccaagtcaggactcgtattaccaaacacaggctgtgacggagtggcagctgcagcggaagtgaatgaaaatacagaacttgaagaggcaccgatggatgacccaaagagtagatgcaccgctaggagcactaacagaagttgatggtccaaaggaaaagcctgattaatgaaccaaatgtggatgtcttgggagcttgccaagtggatccaaatgcaccaccagcaatgccactgctagagcttagagacttggtttctgaagaggaagaacttattccaaaacttgaacctgaactaaaaggtttagctacagatgccgaagacccaaaaatcgaactgccagaggaaaaggttgcattcccagtcaatccaaaagatgaagatgatgcagatgaggaaaactgaatgggcgtgctctgagttgaggtaccaatgccagttcctgcagcagatgcttgggtatttacagcattgaaaggatttaaaccctcagactgtgaagtagatgtggatgtagctggagcactggatccaacgatgtttcttccagtggtcctgactgcagctttagtggcaaaaatacgacttcctgtaccagatactgggaaagacgtaccaccaaaaaaactactacctgtgcttgttgcagatgtacccccaaagatgccattgcctgtgcttgtagctgctgatgtaccaccaaataaacccgtgcccgtgcttgtagttgcagatgtaccaccaaagataccattgcctgtgcttgtacttgcagatgtaccaccgaagataccattgcttgtgcttgtcactggggaaaatgtgctgctgaaaaagccacttccagagttcgtaatagtggaagatataccactggaactgctatttccag

SEQ ID NO: 223 - DNA, 1000 bp, Gossypium hirsutum
  source 1..1000: mol_type = genomic DNA
cttgcagcacttgtaccaccaaaaatactacttccagagccagtaaaagcagatgtactaccaaaaggaaagatacctacattgccaagttttgtatcctgtgtcttagattcagttgcttcccctgaagttgctgataatgttgaaactgaagttgatgggtccccagaggatgaaaacttgaaaacaggtgcacctgtaaaggaagggattgaagcctccacagaggacctgctggtaaagctgatgctggcattcgcagttgcacttgcagaggtgatggtagcagcagaagtgtcatttttggtggcagttgcactggaggaactttgacaattactcgggactagagaaggcaccggagaagagaacgaaaaaggactagaagcacaagacccatgattaaaggtagaaccatctgctgacgtatcaaacttgaacttccagctgatattgaggttgatattgaagttgattggtgttgcgaagtggacataatggcaaactcaggatgcaaatctgcgagctcatttaagtttacgcgctctacatctcatccaaagttgcaaactcatccatcactgagagctcatttgaagatgtgagttcacccaaagatgcgagctcatcaaaagatgtgaactattagtataatttattcaaaaaaattcgaaactcaaatccatatattttgcatgtatgcagatgaggtttgatggtgaaacaagagctacagaaatcaatggtactgagttgcacttcaatagtgtcactggcattctccttctaagcatgccatcaatggcaggaggtaatatatataattttgatatctccatgtagtcatggttttttttttaattatataatctcgtgttataatttcattgtaatagaaccaattaaaaaatattttttaattttaaaattcaggtttgatatagaggcacacttgattcatgaaagtgcatacggtaagattgttgtaatagggatcatg

SEQ ID NO: 224 - DNA, 3696 bp, Synthetic construct
  source 1..3696: mol_type = other DNA
atgaccagcaaccgaaagaacgagaacgagatcatcaacgccctgtccataccggccgtgtcaaaccactccgcccagatgaacctctccaccgacgcgaggatcgaggactccctctgcatcgccgagggcaacaacatcgacccgttcgtgtctgcaagcacggtccagaccggcatcaacatcgcgggccgcatcctgggcgtgctcggcgtgcccttcgcgggtcaaatcgcctctttctactcattcctcgtgggcgagctgtggccgcgcggacgtgacccgtgggaaatcttcctggagcacgttgagcagctcatccggcagcaagtgaccgagaacaccagggacaccgcactggcacggctccagggccttggcaacagcttccgcgcctaccagcagtcgctggaggactggctggagaaccgagacgacgccagaacccgctcagttctgtacacacagtacatcgccctagagctggacttcctcaacgctatgccgctcttcgccatccgtaaccaggaagtaccgcttctgatggtgtacgcacaagcagcgaacctccatctgctcctgctgcgagacgcatctctgttcggcagtgagttcgggctgacgagccaggagatccagcgctactacgagcgccaagtggagaagactcgtgagtacagcgactactgcgcgcgctggtacaacacgggcttgaacaaccttcgcgggacaaacgccgaatcctggcttcgctacaaccagttccgccgcgacctcacgctgggtgtgctggacctggtcgcgctcttcccgtcctacgacacacgggtgtacccaatgaacacgagcgcacagctcacccgtgagatctacacagatcccatcggccgcaccaacgctcccagtggcttcgcaagcacgaattggttcaacaataacgctccttctttctctgccatcgaggccgctgtcatcagaccgccgcacttactcgatttcccggagcagctcactatcttctctgtgttgtcccggtggtcgaacacgcagtacatgaactactgggtgggccacaggctagagagccggaccatccgtggcagtctctcaacctcgacccacggcaacacgaacacgagcatcaaccctgtcactctccagtttacatctagggacgtttacaggacagagtcgttcgctggcattaacattctgttgaccactccggtgaacggcgtcccttgggcccgcttcaactggaggaatcctctgaactcactgcgcggcagccttctctacactatcggctacaccggcgttgggacgcaactcttcgactcggagaccgagctgccgcccgagaccaccgagcggcctaactacgagagttattcacacaggctctccaacatccgcttgatttctgggaacaccttgcgggctccggtgtactcctggacgcaccgcagcgccgacagaactaataccatcagctccgactcgatcacccagatcccgctggtgaaggctcacacgcttcagtcgggcaccacagtcgtcaagggccctggcttcaccggcggcgacatcctgcgtcgcacatctggcggacccttcgccttcagcaacgtgaacttggacttcaatttgtcacagcggtatcgtgccagaatccggtacgccagcactacgaacctgcgaatctatgttactgtggcgggcgagcggatcttcgccgggcaattcgacaagacgatggacgcgggagcacctctgacattccagtcattctcttacgccacgatcaacacggcattcacgtttccggagcgttccagtagcctgaccgtgggcgctgataccttcagtagcgggaacgaggtgtacgttgaccgtttcgagctgatcccggtcaccgccaccacggcgaccttcgaggcggagtatgacttggagcgggctcaggaggccgtcaacgcgctgttcacaaacaccaatcctcgccgcctcaagacgggtgtgactgattaccacattgacgaggtctccaacttggtcgcgtgtctgtccgatgagttctgcctggacgagaagcgggaactgctggagaaggtcaagtacgccaagcgcctctccgacgaaaggaacctcctccaagatcccaactttacttccattaacaagcagccggacttcatctccaccaacgagcagtccaacttcacctcaatccacgagcagtcggagcacgggtggtggggcagcgagaacatcaccatccaagagggcaacgacgtcttcaaggagaactacgtgatcctgcccggcaccttcaacgagtgttacccgacctatctctaccagaagattggcgaagcggaactcaaggcttacacccgttaccaactgagtggctacattgaggactcacaagacctggaaatctacctgatccgctacaacgccaagcacgagaccctcgacgtgcctggcacggagtccgtctggcccttgagcgtggagtctcctatcggtcgttgcggcgagcccaatcgctgcgctccgcactttgagtggaatcctgatttggattgctcctgccgagacggtgagaaatgcgcccaccactcgcaccacttcagcctagacatcgacgtgggctgcatcgacctgcacgagaacttgggcgtctgggtcgtgttcaagatcaagacacaggagggccatgctcggcttgggaacctggagttcatcgaggagaagccactgctgggtgaagccttgtcacgggtgaaacgcgccgagaagaagtggcgggacaaacgggagaagctccagttggagacaaagcgtgtgtacacagaggccaaggaggccgtggatgccttgttcgtggacagtcagtacgacaggctgcaagcggacaccaacatcgggatgatccacgcggctgataagcttgttcacagaatccgcgaggcgtacctgtcagagcttagcgtgatcccaggcgtcaacgccgaaatcttcgaggaactggagggccgcattatcacggcaatctcactttatgacgcgaggaatgtggtcaagaacggtgacttcaacaacggcttggcgtgttggaacgttaaagggcacgtggatgtacaacagtcacaccacagaagtgtcttggtcatcccggagtgggaggcggaagtgagccaggccgtccgggtctgccctgggcgcggttacatcctccgcgtgacagcgtacaaggagggctacggtgagggctgcgtgacgatccacgagattgagaacaacacggacgagcttaagttcaagaactgcgaggaggaggaagtgtacccgacagacaccggcacctgcaacgactacaccgcccaccaagggaccgccgcctgcaacagccgcaacgcgggctatgaagatgcgtacgaggttgataccaccgcctcagtgaactacaaaccgacttatgaggaggagacatacacggacgtcaggcgcgacaaccattgtgagtacgaccgtggctacgtgaactatccgccggtgccagcgggctacatgacgaaggagctagaatacttccctgagacggacaaggtgtggattgaaatcggcgagaccgagggcaagtttatcgtggattctgtcgagctgctgctaatggaggagtag

SEQ ID NO: 225 - protein, 1231 aa, Synthetic construct
MTSNRKNENEIINALSIPAVSNHSAQMNLSTDARIEDSLCIAEGNNIDPFVSASTVQTGINIAGRILGVLGVPFAGQIASFYSFLVGELWPRGRDPWEIFLEHVEQLIRQQVTENTRDTALARLQGLGNSFRAYQQSLEDWLENRDDARTRSVLYTQYIALELDFLNAMPLFAIRNQEVPLLMVYAQAANLHLLLLRDASLFGSEFGLTSQEIQRYYERQVEKTREYSDYCARWYNTGLNNLRGTNAESWLRYNQFRRDLTLGVLDLVALFPSYDTRVYPMNTSAQLTREIYTDPIGRTNAPSGFASTNWFNNNAPSFSAIEAAVIRPPHLLDFPEQLTIFSVLSRWSNTQYMNYWVGHRLESRTIRGSLSTSTHGNTNTSINPVTLQFTSRDVYRTESFAGINILLTTPVNGVPWARFNWRNPLNSLRGSLLYTIGYTGVGTQLFDSETELPPETTERPNYESYSHRLSNIRLISGNTLRAPVYSWTHRSADRTNTISSDSITQIPLVKAHTLQSGTTVVKGPGFTGGDILRRTSGGPFAFSNVNLDFNLSQRYRARIRYASTTNLRIYVTVAGERIFAGQFDKTMDAGAPLTFQSFSYATINTAFTFPERSSSLTVGADTFSSGNEVYVDRFELIPVTATTATFEAEYDLERAQEAVNALFTNTNPRRLKTGVTDYHIDEVSNLVACLSDEFCLDEKRELLEKVKYAKRLSDERNLLQDPNFTSINKQPDFISTNEQSNFTSIHEQSEHGWWGSENITIQEGNDVFKENYVILPGTFNECYPTYLYQKIGEAELKAYTRYQLSGYIEDSQDLEIYLIRYNAKHETLDVPGTESVWPLSVESPIGRCGEPNRCAPHFEWNPDLDCSCRDGEKCAHHSHHFSLDIDVGCIDLHENLGVWVVFKIKTQEGHARLGNLEFIEEKPLLGEALSRVKRAEKKWRDKREKLQLETKRVYTEAKEAVDALFVDSQYDRLQADTNIGMIHAADKLVHRIREAYLSELSVIPGVNAEIFEELEGRIITAISLYDARNVVKNGDFNNGLACWNVKGHVDVQQSHHRSVLVIPEWEAEVSQAVRVCPGRGYILRVTAYKEGYGEGCVTIHEIENNTDELKFKNCEEEEVYPTDTGTCNDYTAHQGTAACNSRNAGYEDAYEVDTTASVNYKPTYEEETYTDVRRDNHCEYDRGYVNYPPVPAGYMTKELEYFPETDKVWIEIGETEGKFIVDSVELLLMEE

SEQ ID NO: 226 - DNA, 3501 bp, Synthetic construct
  source 1..3501: mol_type = other DNA
atggctgagatcaacaaccagaaccagtgcgtcccgtacaactgcctgagcaaccctaaggagatcatcctgggtgaggaacgcctggagaccggcaacaccgtagccgacattagcctgggcctcatcaacttcctctacagcaacttcgtgcccggcggtggcttcatcgtgggcctcctggagcttatctggggcttcatcggcccgtcccagtgggacatcttcctcgcccagatcgagcaactgatcagccagcggatcgaggagttcgctaggaaccaggccatctcccgcctggagggactctccaacctctacaaggtgtacgtgcgcgcgttcagcgactgggagaaggacccgaccaacccggccctccgcgaggaaatgcgtatccagttcaacgatatgaactcggccctcatcaccgccatcccgctcttccgcgtgcagaactacgaggtggccctcctgtccgtgtacgttcaagccgccaacctccacctctccatcctccgcgacgtgagcgtgttcggcgagcgctggggctacgacaccgccaccatcaacaaccgctactccgacctcacctccctcatccacgtttacaccaaccactgcgtggacacgtacaaccagggcctccgccgcctggagggccgcttcctctccgactggatcgtgtacaaccgcttccgccgccagctcaccatctccgtcctggacatcgtcgccttctttcccaactacgacatccgcacctaccctatccagaccgccacccagctcacccgcgaggtctacctcgacctcccgttcatcaacgagaacctcagcccggccgccgtctacccgaccttctccgccgctgagtccgccatcattcgcagcccgcacctcgtggacttcctcaactccttcaccatctacaccgactccctcgcccgcagcgcctactggggcggtcacctcgtgaactccttccgcaccggcaccactaccaacctcatccgcagcccgctctacggccgcgagggcaacaccgagcgcccggtgaccatcaccgccagcccgagcgtgcccatcttccgcaccctcagctaccccaccggcctggacaacagcaaccctgtggcgggcatcgagggcgtggagttccagaacaccatctccaggagcatctaccgcaagagcggccctatcgacagcttcagcgagctgcctcctcaggacgccagcgtgagccctgccatcggctacagccacaggctgtgccacgccaccttcctggagcgcatcagcggccctcgcatcgcgggcaccgtgttctcgtggacccaccgcagcgcctctcctacgaacgaggtgtctcctagtcgcatcacccagatcccttgggtcaaggcccacaccctggctagtggcgctagtgtcatcaagggccctggcttcaccggtggtgacatcctgaccaggaactctatgggcgagctgggcactctgagggtcactttcactggccgcctgcctcagtcttactacatccgcttccgctacgctagtgtcgctaaccgctctggtactttccgctactctcagcctccgtcttacggtatctctttccctaagactatggacgctggtgagcctctgaccagtaggagcttcgctcacactactctgttcactcctatcactttctctagggctcaggaggagttcgacctgtacatccagtctggtgtgtacatcgacaggatcgagttcatccccgtgaccgccacgttcgaggccgagtacgaccttgagcgcgcccagaaggtggtgaacgccctcttcactagcactaaccagctaggcctgaagactgacgtgaccgactaccacatcgaccaagtgagcaacctagtggcctgcctctccgacgagttctgcctcgacgagaagcgcgagctgtccgagaaggtgaagcacgccaagcgcctctccgacgagcgcaacctgctccaggaccccaacttcaggggcatcaacaggcagcccgaccgcggctggcgcggctccaccgacatcaccatccagggcggtgacgacgtattcaaggagaactacgttaccctccccggcaccttcgacgagtgttaccccacctacctctaccagaagatcgacgagtccaagctgaaggcctacacccgctaccagctccgcggctacatcgaggactcccaggacctggaaatctacctcatccgctacaacgccaagcacgagatcgtgaacgtgcctggcaccggcagcctctggcctctcagcgtggagaaccagatcggcccttgcggcgagcctaaccgctgcgcccctcacctcgagtggaaccctgacctccactgctcgtgcagggacggcgagaagtgcgcccaccatagccaccacttctctctggacatcgacgtgggctgcaccgacctgaacgaggacctgggcgtgtgggttatcttcaagatcaagacccaggacggtcacgccaggctgggtaacctggagttccttgaggaaaagcctctgctgggtgaggccctggccagggtcaagagggctgagaagaaatggagggataagagggagaccctgcagctggagaccactatcgtctacaaggaggctaaggagtctgtcgatgctctgttcgtcaactctcagtacgatagactgcaagctgataccaacatcgctatgatccacgctgcggataagcgggtccaccggatccgggaggcttaccttccggagctttctgtcatcccgggtgtcaacgctgcgatcttcgaggaacttgaggaacggatcttcactgcgtttagtctttacgatgcgcggaacatcatcaagaacggggacttcaacaatggtctgctgtgctggaacgtcaagggtcatgtcgaggtcgaggaacaaaacaatcatcgtagtgtccttgtcattcctgagtgggaggcggaggtctctcaagaggtccgtgtttgcccggggcgtgggtacattcttcgtgttactgcgtacaaggaggggtacggggaggggtgcgttactattcatgagattgagaacaatactgatgagcttaagttcaacaattgtgttgaggaggaggtttacccgaacaatactgttacgtgcatcaactacacggcaacgcaagaggaatacgaggggacgtacacctcgcgtaatagagggtatgatgaggcgtacggaaacaacccgtcggttccagcagattatgcctcggtttatgaggagaagtcgtacacggatagacgacgcgagaatccatgtgagtcaaatcgaggatacggagattacacaccattaccagcaggatacgttacaaaggagttggaatacttcccggaaacagataaagtttggattgaaatcggagaaacagaaggaacattcatcgtcgactcagtagaattgttgttgatggaagaatga

SEQ ID NO: 227 - protein, 1166 aa, Synthetic construct
MAEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAAVYPTFSAAESAIIRSPHLVDFLNSFTIYTDSLARSAYWGGHLVNSFRTGTTTNLIRSPLYGREGNTERPVTITASPSVPIFRTLSYPTGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 228 - DNA, 2397 bp, Synthetic construct
  source 1..2397: mol_type = other DNA
atggctaagcagaacaacaacttctccgtgcgcgcgctcccgtcgttcatcgacgtcttcaacggcatctacgggttcgccaccggcatccaggacatcttcaacatgatcttcggcaccgacacgggcgacctcacgctggaggaggtgctcaagaaccaggaactgctctacgacatctcgggcaagctggagggcatctccggcgacctcagcgaaattatcgcgcaagggaacctcaacacggagctggcgaaagagctgctcaagatcgccaacgagcagaacaacgtgctgacggacgtgaacaacaagctcaacgcgatcaactcgatgctccacatctacctcccaaagattacgaacatgctgtccgacgtcatgaagcagaactatgccctcagcctccagatcgagtacctctcgaagcagctccaggagatttcggataagctggacgtcatcaacctgaacgtgctgataaactccacgctcacggagatcactccggcctaccagcgcatcaagtacgtgaacgagaagttcgacgagctgacactggcgactgagaagaccctccgcgccaagcaagggtccgaggacatcattgcgaacgacacgctggagaacttgacggagttgaccgagctggccaagagcgtgacgaagaacgacatggacagcttcgagttctacctccacactttccacgacgtgctgatcggcaacaacctcttcggccggagcgcgctcaagaccgcagccgagctgattacgaaggacgagatcaagacctccgggtccgagatcggaaaggtgtacagcttcctgatcgtcctcacgtgcttacaagctaaggcgttcctcaccctgaccgcctgccgtaagctgttgggcctgtccgacatcgactacaccaacatcctcaaccagcacctcaacgacgagaagaacgtctttcgggacaacatcctcccgacactgagcaacaagttcagtaacccgaactacgtcaagacgattggcagcgacaactacgccaaggttatcctagaggccgagcccggttacgccctggtcgggttcgagatcatcaacgaccgcatcccggtcctcaaggcgtacaaggccaagctcaagcagaactaccaagtggaccaccagagcctcagcgagatcgtgtacctggacatcgacaagttgttctgcccgaagaactcggagcagaagtattacaccaagagcctgaccttcccggacggctacgttattacaaagatcacgttcgagaagaagctcaacaacctccgttacgaggcaactgccaacttctacgacccgtccaccggcgacattgacttgaacgagaagcaagtcgagtccacgttcctccaggccgactacatcagcatcaacgtcagcgacgacgacggcgtgtacatgccgctcggagtcatcagcgagaccttcctcagcccgatcaactcgttcgagctggaggtggacgagaagtccaagattctcaccctgacctgcaagagctacctccgggaatacctcctggagagcgacctcatcaataaggagacttcgctcatagctccgcccaacgtcttcatctccaacatcgtcgagaactggaacatcgaggccgacaacctggagccgtgggtggcaaacaacaagaacgcctacgtggactccaccggcgggatcgagggaagcaaggccctgttcacccagggcgacggtgagttctcgcagttcatcggcgacaagctcaagcccaacacggactacatcatccagtacaccgtcaagggcaagccagcgatctacctcaagaacaagaacaccgggtacaccatgtacgaggacacgaacggcagcagcgaggagttccagaccatcgcggtcaactacacctccgagaccgatccctcccagacccaccttgtcttcaagtcccagagcggctacgaggcgtggggcgacaacttcatcatcctggagtgcaaggctttcgagactcccgagggcccggagcttatcaagttcgatgactggatttcgtttggcaccacctacatccgggacgacgtgctaaccatcgacccgtcgcgcggcggctacttccgccaaagcctcaagctggactcgtactccacgtacaacctatctttcagcttctcgggcctgtgggcgaaggtgattatcaagaactcgcacggcgtggtcctgttcgagaaggtctcccagcagtcgtcatacgtggacatcagcgagtccttcaccaccaccagcaacaaggagggcttcttcatcgagctgaccggcgactcgcgcggcgggttcggctccttccgcgacttctccatgaaggagaaattcgagtga

SEQ ID NO: 229 - protein, 798 aa, Synthetic construct
MAKQNNNFSVRALPSFIDVFNGIYGFATGIQDIFNMIFGTDTGDLTLEEVLKNQELLYDISGKLEGISGDLSEIIAQGNLNTELAKELLKIANEQNNVLTDVNNKLNAINSMLHIYLPKITNMLSDVMKQNYALSLQIEYLSKQLQEISDKLDVINLNVLINSTLTEITPAYQRIKYVNEKFDELTLATEKTLRAKQGSEDIIANDTLENLTELTELAKSVTKNDMDSFEFYLHTFHDVLIGNNLFGRSALKTAAELITKDEIKTSGSEIGKVYSFLIVLTCLQAKAFLTLTACRKLLGLSDIDYTNILNQHLNDEKNVFRDNILPTLSNKFSNPNYVKTIGSDNYAKVILEAEPGYALVGFEIINDRIPVLKAYKAKLKQNYQVDHQSLSEIVYLDIDKLFCPKNSEQKYYTKSLTFPDGYVITKITFEKKLNNLRYEATANFYDPSTGDIDLNEKQVESTFLQADYISINVSDDDGVYMPLGVISETFLSPINSFELEVDEKSKILTLTCKSYLREYLLESDLINKETSLIAPPNVFISNIVENWNIEADNLEPWVANNKNAYVDSTGGIEGSKALFTQGDGEFSQFIGDKLKPNTDYIIQYTVKGKPAIYLKNKNTGYTMYEDTNGSSEEFQTIAVNYTSETDPSQTHLVFKSQSGYEAWGDNFIILECKAFETPEGPELIKFDDWISFGTTYIRDDVLTIDPSRGGYFRQSLKLDSYSTYNLSFSFSGLWAKVIIKNSHGVVLFEKVSQQSSYVDISESFTTTSNKEGFFIELTGDSRGGFGSFRDFSMKEKFE

SEQ ID NO: 230 - DNA, 1908 bp, Synthetic construct
  source 1..1908: mol_type = other DNA
atggacaactccgtcctgaactctggtcgcaccaccatctgcgacgcctacaacgtcgcggcgcatgatccattcagcttccagcacaagagcctcgacactgttcagaaggagtggacggagtggaagaagaacaaccacagcctgtacctggaccccatcgtcggcacggtggccagcttccttctcaagaaggtcggctctctcgtcgggaagcgcatcctctcggaactccgcaacctgatctttccatctggctccaccaacctcatgcaagacatcctcagggagaccgagaagtttctcaaccagcgcctcaacactgatacccttgctcgcgtcaacgctgagctgacgggtctgcaagcaaacgtggaggagttcaaccgccaagtggacaacttcctcaaccccaaccgcaatgcggtgcctctgtccatcacttcttccgtgaacaccatgcaacaactgttcctcaaccgcttgcctcagttccagatgcaaggctaccagctgctcctgctgccactctttgctcaggctgccaacctgcacctctccttcattcgtgacgtgatcctcaacgctgacgagtggggcatctctgcagccacgctgaggacctaccgcgactacctgaagaactacaccagggactactccaactattgcatcaacacctaccagtcggccttcaagggcctcaatacgaggcttcacgacatgctggagttcaggacctacatgttcctgaacgtgttcgagtacgtcagcatctggtcgctcttcaagtaccagagcctgctggtgtccagcggcgccaacctctacgccagcggctctggtccccaacaaactcagagcttcaccagccaggactggccattcctgtattcgttgttccaagtcaactccaactacgtcctcaacggcttctctggtgctcgcctctccaacaccttccccaacattgttggcctccccggctccaccacaactcatgctctgcttgctgccagagtgaactactccggcggcatctcgagcggcgacattggtgcatcgccgttcaaccagaacttcaactgctccaccttcctgccgccgctgctcaccccgttcgtgaggtcctggctcgacagcggctccgaccgcgagggcgtggccaccgtcaccaactggcaaaccgagtccttcgagaccacccttggcctccggagcggcgccttcacggcgcgtgggaattctaactacttccccgactacttcatcaggaacatctctggtgttcctctcgtcgtccgcaacgaggacctccgccgtccactgcactacaacgagatcaggaacatcgcctctccgtccgggacgcccggaggtgcaagggcgtacatggtgagcgtccataacaggaagaacaacatccacgctgtgcatgagaacggctccatgatccacctggcgcccaatgattacaccggcttcaccatctctccaatccacgccacccaagtgaacaaccagacacgcaccttcatctccgagaagttcggcaaccagggcgactccctgaggttcgagcagaacaacaccaccgccaggtacaccctgcgcggcaacggcaacagctacaacctgtacctgcgcgtcagctccattggcaactccaccatcagggtcaccatcaacgggagggtgtacacagccaccaatgtgaacacgacgaccaacaatgatggcgtcaacgacaacggcgcccgcttcagcgacatcaacattggcaacgtggtggccagcagcaactccgacgtcccgctggacatcaacgtgaccctgaactctggcacccagttcgacctcatgaacatcatgctggtgccaactaacatctcgccgctgtactgatag

SEQ ID NO: 231 - protein, 634 aa, unidentified
  source 1..634: note = Bacillus thuringiensis subsp. Kumamotoensis
MDNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISPLY

SEQ ID NO: 232 - DNA, 2267 bp, Synthetic construct
  source 1..2267: mol_type = other DNA
cttgtcattggtgcagacaggttcggggagtatggcctatgggtggtggtagagagaaagtaacagcgaaatcttagggtggtccgagaaatgagattgttaagtcctgatttaatgtgctgagttcagttgatggagaggaaaatggcaaagatttgaggggagaggattttgtggatcctaacattaaacagaatcgttctactaaaaggggtaagggtagtggtggaagagatatttttaaaaaagctaaggtaaaatctgggcaaagttttaacgtggcccaggggaataaaaaagggactatagggcctagtggtagtcaacaggccgggcgggaaagtattcatttgaaatgggtttattcgtattaacagctactctaggaggaatgtttttatgcggtgctaacgatttaataactatctttgtagctccagaatgtttcagtttatgctcctacctattatctggatataccaagaaagatgtacggtctaatgaggctactacgaaatatttactcatgggtggggcaagctcttctattctggttcatggtttctcttggctatatggttcatccgggggagagatcgagcttcaagaaatagtgaatggtcttatcaatacacaaatgtataactccccaggaatttcaattgcgcttatattcatcactgtaggaattgggttcaagctttccccagccccttctcatcaatggactcctgacgtatacgaaggagttcttttaggcttttgcattttgttgaaaattcttattttcacaggactttgcttatcataattattgacagccaactggtgaaaatggtgccacgagagctcccatcagcattgatttggctagggatagttctttaagtgctaagtcaatcatcttgcaaattgacaataaatctcaacaggtttcagcaagtaaggcctccttcattattttggttgcttctttttcattaatgatgttgcttgtttgcacctggaaaacatgcaccgttcttcatcatgcaccttggatatggtcttgagtgctctctgactgcccgtcatctttcgaattggtgtctgattgagtgcacccgcctaagttcaagatgccggagtttgaatagtataacgggactagttgtcttgaggctcatattactatgttctgtcgatataatcatccatgggcttcgcatccaccattgcaaggcccaaccacctagcactgtgatgggtccaaacgcaaaggtgatccctttaggccaaatggatggagacgctataagattggtcaccgcaagaaaagtgtggatcgataggccctgtagttgatgatggggatgatcacgatgaccctgcctatcctccaagtttcaccccaactaacatccaagtgcaaccagaggtgtacccgcagagggcacctattaccattagatcgactgcaagtgtaggtaatgttggacagggtagatcagggtgttcacggtgtggtagactttactatggtccttttcgggcaggcgaaaatgtttgttataaatgcggcactccagatcattttgtacgagaatgtccagaggtggctaatcgagagaatcgaatcacttacgaaaaaagattattgttactaagttttcatgttttatgccatgcgtagccatgtttttaataaagggttgaagacttgaatgacaagttagcgtaaagcattttttttctttcatgttggcacatttgatgcactatgtcttcgtctatttttcaaacatactgttggaagaattatgattcacgtgttgtttaagatcaaaaagtgcatgcctaactaatacttatcagaaacaaataatgcaatgagtcatatctctataaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatatgatatctccactgacgtaagggatgacgcacaatcccactatccttcgcaagacccttcctctatataaggaagttcatttcatttggagaggacacgctgacaagctgactctagcagatctccatggtccgtcctgtagaaacc

SEQ ID NO: 233 - DNA, 1360 bp, Synthetic construct
  source 1..1360: mol_type = other DNA
ccaactaacatctcgccgctgtactgataggagctctgatccccatgggaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatatccccggggcggccgctctagaactagtggatctgcactgaaatcccatccatttagcaaccttaaaagtaataaacttaaactaagtgtactccttatcaaaacataaaccaagagatgaggataacagcgttcggccaaccccaataccacctataatcaaagcatgcgatgtcacgaacgttttcctgacggcaatcgacacaaagaagagattaaaaagggagaccactatagaaagaagaaagagaatcggcaccactgtgaaactacatttgataaccgtattagcaaggctccaccattgataaaaactcgacaaccaagaggaactcacttaccttgatcaaagtagggttgttggtactaaggctcaaagcaaggaaaatgtctaaatgagtgagttgggcttttttaatttaattaaactgaaaaataagaaaggatgttgccaattttgtgtggcaaggacggacaagaggtttcgacccaacctagagaagacattaaaactgcagaactgggctcgcttgttcaggatcgggctttaaaggagaatggtttgatggaccagaatagagacaaaccaatacactcagattttgatcttccttattctccctcaactattgattttgttttgggtaatgatggtgtagcaggggatcgggttttaggtgtttttcaaggcatgactgttactcattttaatccgacttgcgatgaccataatgagatggaggctgtactgaaggatgaagtattggatccaaataggcattcgacatgtttgataaaactcttagttacaaaattcttagtaaacgaatccataaggttatctttagatgttattttcatcacatctataattctttcagctactgcctcttgtatcaagtgatactttcagtcagtatattcgtcttgcagcactgttatcacaatacagtgttatatctttttctatatcaggaatgacttcaagatcggttaagaacttttaaagccatattgtttctttcgtagcctcagaagcaacca

SEQ ID NO: 234 - DNA, 23 bp, Synthetic construct
  source 1..23: mol_type = other DNA
cagacaaacccttatgtccgaaa

SEQ ID NO: 235 - DNA, 19 bp, synthetic construct
  source 1..19: mol_type = other DNA
ctgcgaacactccgcacaa

SEQ ID NO: 236 - DNA, 19 bp, synthetic construct
  source 1..19: mol_type = other DNA
ttcttgcttgctaccccct

SEQ ID NO: 237 - DNA, 20 bp, synthetic construct
  source 1..20: mol_type = other DNA
tgcgtccaatgcctgctcgc